(19) United States
(12) Patent Application Publication
(10) Pub. No.: US 2020/0325177 A1
(43) Pub. Date: Oct. 15, 2020

(54) METHODS OF MAKING STREPTOGRAMIN COMPOSITIONS AND THE USE THEREOF

(71) Applicant: THE REGENTS OF THE UNIVERSITY OF CALIFORNIA, Oakland, CA (US)

(72) Inventors: Ian B. Seiple, San Francisco, CA (US); Qi Li, San Francisco, CA (US)

(21) Appl. No.: 16/635,070

(22) PCT Filed: Jul. 31, 2018
(86) PCT No.: PCT/US18/44708

§ 371 Date: Jan. 29, 2020

Related U.S. Application Data
(60) Provisional application No. 62/539,989, filed on Aug. 1, 2017.

(51) Int. Cl. C07K 11/02; C07K 1/02

(52) U.S. Cl. CPC C07K 11/02; A61K 38/00; C07K 1/02

(57) ABSTRACT

Disclosed herein, inter alia, are methods of making and using streptogramin compositions.

### CROSS-REFERENCES TO RELATED APPLICATIONS

This application claims the benefit of U.S. Provisional Application No. 62/539,989, filed on Aug. 1, 2017, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant no. TR001872 awarded by the National Institutes of Health. The government has certain rights in the invention.

### BACKGROUND

Streptogramin antibiotics are used clinically to treat bacterial infections, but their poor physicochemical properties and narrow spectra of activity have limited their clinical utility. New methods to chemically modify streptogramin compounds would enable structural optimization to overcome these limitations as well as to combat growing resistance to the class. Disclosed herein, inter alia, are solutions to these and other problems within the art.

### BRIEF SUMMARY OF THE INVENTION

In an aspect is provided a compound, or salt thereof, having the formula:

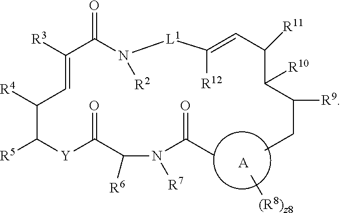

Y is —O— or —NH—. L¹ is a bond, substituted or unsubstituted alkylene, or substituted or unsubstituted heteroalkylene. R² is hydrogen or unsubstituted C₁-C₃ alkyl. R³, R⁴, and R⁵ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃, —SO₄, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —OPO₃H, —OSO₃H, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OC₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or an amino acid side chain. R and R⁷ may optionally be joined to form a substituted or unsubstituted heterocycloalkyl or substituted or unsubstituted heteroaryl. R⁸ is oxo, halogen, —CCl₃, —CBr₃, —CF₃, —CI₃, CHC₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Ring A is cycloalkylene, heterocycloalkylene, arylene, or heteroarylene. z8 is an integer from 0 to 10. R⁹, R¹⁰, R¹¹, and R¹² are each independently hydrogen, oxo, halogen, —CCl₃, —CBr₃, —CF₃, —C₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OC₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In an aspect is provided a compound, or salt thereof, having the formula:

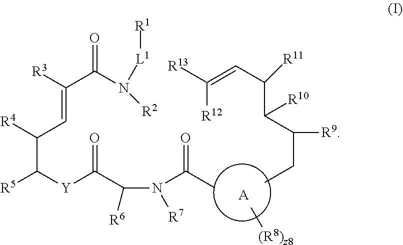

R¹ and R¹³ are independently a bioconjugate reactive moiety or a leaving group. Y is —O— or —NH—. L¹ is a bond, substituted or unsubstituted alkylene, or substituted or unsubstituted heteroalkylene. R² is hydrogen or unsubstituted C₁-C₃ alkyl. R³, R⁴, and R⁵ are independently hydrogen, oxo, halogen, —CCl₃, —CBr₃, —CF₃, —CI₃, CHC₂, —CHBr₂, —CHF₂, —CHI₂, —CH₂CL, —CH₂Br, —CH₂F, —CH₂I, —CN, —OH, —NH₂, —COOH, —CONH₂, —NO₂, —SH, —SO₃, —SO₄, —SO₂NH₂, —NHNH₂, —ONH₂, —NHC(O)NHNH₂, —NHC(O)NH₂, —NHSO₂H, —NHC(O), —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —OPO₃H, —OSO₃H, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OC₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or an amino acid side chain. R⁷ is hydrogen, —CH₂COOH, —CONH₂, —OH, —SH, —NO₂, —NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶ and R⁷ may optionally be joined to form a substituted or unsubstituted heterocycloalkyl or substituted or unsubstituted heteroaryl. R⁸ is oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Ring A is cycloalkylene, heterocycloalkylene, arylene, or heteroarylene. z8 is an integer from 0 to 10. R⁹, R¹⁰, R¹¹, and R¹² are each independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COO, —CONH₂, —NO₂, —S, —SO₃, —SO₄, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In an aspect is provided a compound, or salt thereof, having the formula:

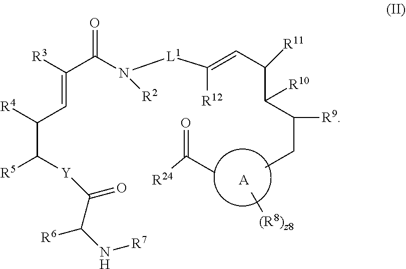

R²⁴ is a bioconjugate reactive moiety or leaving group. Y is —O— or —NH—. L¹ is a bond, substituted or unsubstituted alkylene, or substituted or unsubstituted heteroalkylene. R² is hydrogen or unsubstituted C₁-C₃ alkyl. R³, R⁴, and R⁵ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —OPO₃H, —OSO₃H, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OC₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or an amino acid side chain. R and R⁷ may optionally be joined to form a substituted or unsubstituted heterocycloalkyl or substituted or unsubstituted heteroaryl. R⁸ is oxo, halogen, —CCl₃, —CBr₃, —CF₃, —CI₃, CHC₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OC₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Ring A is cycloalkylene, heterocycloalkylene, arylene, or heteroarylene. z8 is an integer from 0 to 10. R⁹, R¹⁰, R¹¹, and R¹² are each independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COO, —CONH₂, —NO₂, —SH, —SO₃, —SO₄, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OC₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In an aspect is provided a method of making a compound, or salt thereof, having the formula:

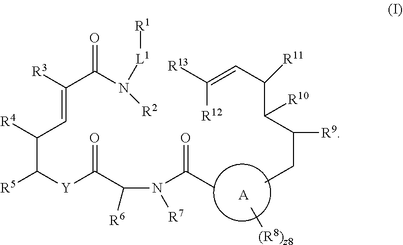

The method including mixing compound A and compound B together in a reaction vessel; wherein compound A has the formula:

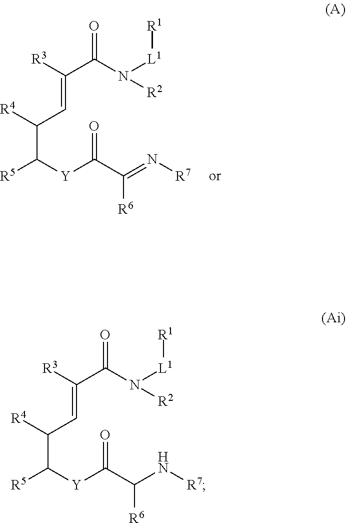

and compound B has the formula:

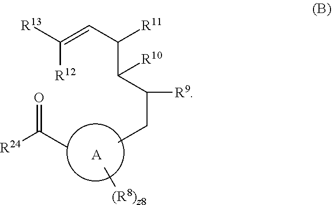

R¹, R¹³, and R²⁴ are independently a bioconjugate reactive moiety or a leaving group. Y is —O— or —NH—. L¹ is a bond, substituted or unsubstituted alkylene, or substituted or unsubstituted heteroalkylene. R² is hydrogen or unsubstituted C₁-C₃ alkyl. R³, R⁴, and R⁵ are independently hydrogen, oxo, halogen, —CCl₃, —CBr₃, —CF₃, —CI₃, CHC₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃, —SO₄, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —OPO₃H, —OSO₃H, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —S, —SO₃, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OC₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or an amino acid side chain. R⁷ is hydrogen, —CH₂COOH, —CONH₂, —OH, —SH, —NO₂, —NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶ and R⁷ may optionally be joined to form a substituted or unsubstituted heterocycloalkyl or substituted or unsubstituted heteroaryl. R⁸ is oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Ring A is cycloalkylene, heterocycloalkylene, arylene, or heteroarylene. z8 is an integer from 0 to 10. R⁹, R¹⁰, R¹¹, and R¹² are each independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In another aspect is provided a pharmaceutical composition including a compound as described herein, or pharmaceutically acceptable salt thereof, and a pharmaceutically acceptable excipient.

In an aspect is provided a method of treating an infectious disease, said method comprising administering to a subject in need thereof an effective amount of a compound as described herein.

### BRIEF DESCRIPTION OF THE DRAWINGS

FIG. 1. Selected natural and semisynthetic group A and group B streptogramin antibiotics with an example of a semisynthetic modification.

FIG. 2. Synthesis of virginiamycin M1 by the convergent assembly of 7 chemical building blocks.

FIG. 3. Syntheses of virginiamycin M2 (5) and madumycin II (6).

FIG. 4. Synthesis of madumycins I (4) and II (5) by the convergent assembly of 7 simple building blocks. Reagents and conditions: (a) 10 (0.5 equiv), TfOH (0.45 equiv), DCM, −78° C., 1 h, then a solution of isopropanol (1.1 equiv), 8 (1 equiv), 9 (1.25 equiv) in DCM (slow addition over 2 h), 1.5 h, 94%, 87% ee; (b) propargylamine (4 equiv), AlMe₃ (4 equiv), 0° C. to 23° C., then 11, DCM, 40° C., 3 h, 90%; (c) CuCN (2 equiv), n-BuLi (4.2 equiv), Bu₃SnH (4.2 equiv), 1 h, −78° C., 100%; (d) 12 (1 equiv), 13 (1.5 equiv), DCC (1.6 equiv), DMAP (0.2 equiv), 6 h, then Et₂NH (480 equiv), DCM, 23° C., 3 h, 88%; (e) 16 (1.1 equiv), TiCl₄ (1.2 equiv), ⁱPr₂EtN (1.2 equiv), 2 h, 15 (1 equiv, slow addition over 30 min), 10 min, 64%; (f) 17 (1 equiv), 2,6-lutidine (2 equiv), TBSOTf (1.2 equiv), DCM, 0° C., 30 min, 92%; (g) 19 (2 equiv), n-BuLi (4 equiv), 30 min, then 18 (1 equiv, slow addition over 30 min), THF, −78° C., 30 min, 84%; (h) 14 (1 equiv), 20 (1.1 equiv), ⁱPr₂EtN (2 equiv), HATU (1.5 equiv), DCM, 23° C., 5 h, 88%; (i) 21 (1 equiv), JackiePhos (0.2 equiv), Pd₂dba₃ (0.1 equiv), toluene, 50° C., 3 h, 62%; (j) Bu₄NF (10 equiv), Im.HCl (10 equiv), THF, 23° C., 12 h, 91%; (k) 4 (1 equiv), Et₂BOMe (1.2 equiv), 15 min, then NaBH₄ (2 equiv), THF:MeOH 4:1 (v/v), −78° C., 3 h, 86%. TBS=tert-butyldimethylsilyl, TfO=trifluoromethanesulfonate, DCC=dicyclohexylcarbodiimide, DMAP=4-dimethylaminopyridine, Fmoc=9-fluorenylmethoxycarbonyl Im=imidazole, DCM=dichloromethane, THF=tetrahydrofuran.

FIG. 5. Syntheses of virginiamycins M1 (1) and M2 (3). Reagents and conditions: (a) 22 (1.5 equiv), 12 (1 equiv), DCC (1.6 equiv), DMAP (0.2 equiv), 6 h, then Et₂NH (480 equiv), DCM, 23° C., 3 h, 88%; (b) 23 (1 equiv), 20 (1.1 equiv), ⁱPr₂EtN (2 equiv), HATU (1.5 equiv), DCM, 23° C., 5 h, 87%; (c) JackiePhos (0.2 equiv), Pd₂dba₃ (0.1 equiv), toluene, 50° C., 3 h, 59%; (d) 24 (1 equiv), Bu₄NF (10 equiv), Im.HCl (10 equiv), THF, 23° C., 12 h, 82%; (e) PhIO (1.1 equiv), DCM, 23° C., 30 min, 92%; (f) 20 (1.25 equiv), Ghosez reagent (1.3 equiv), 2,6-lutidine (2.5 equiv), 2 h, then 25 (1 equiv), DCM, 23° C., 12 h, 65%; (g) JackiePhos (0.3 equiv), Pd₂dba₃ (0.15 equiv), toluene, 80° C., 24 h, 47%; (h) 26 (1 equiv), Bu₄NF (10 equiv), Im.HCl (10 equiv), THF, 23° C., 12 h, 85%. TfO=trifluoromethanesulfonate, Fmoc=9-fluorenylmethoxycarbonyl, HATU=1-[Bis(dimethylamino)methylene]-1H-1,2,3-triazolo[4,5-b]pyridinium-3-oxidhexafluorophosphate, TBS=tert-butyldimethylsilyl TMS=trimethylsilyl, dba=dibenzylideneacetone, Im=imidazole, DCM=dichloromethane, THF=tetrahydrofuran.

FIGS. 6A-6E. Chemical structures, names, and molecular information.

FIGS. 7A-7B. 2.8 Angstrom resolution cryo-EM structure of SA0110224 bound to large subunit of the E. coli ribosome; density contoured to 1 sigma; data collected at 200 kEV on a Talos Artica instrument; the isoquinoline sidechain reaches into the P-site of the 70S subunit, a binding site not known to be occupied by any antibiotics. FIG. 7B depicts the structure of SAO110224.

### DETAILED DESCRIPTION

Streptogramin antibiotics are used clinically to treat bacterial infections (e.g., Gram-positive bacterial infections), but their poor physicochemical properties and narrow spectra of activity have limited their clinical utility. New methods to chemically modify streptogramins would enable structural optimization to overcome these limitations as well as to combat growing resistance to the class. Herein we report a modular, scalable synthesis of group A streptogramin compositions that proceeds in 7-8 linear steps from simple chemical building blocks.

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbon atoms (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, Sn, or P). A heteroalkyl moiety may include two optionally different heteroatoms (e.g., O, N, S, Si, Sn, or P). A heteroalkyl moiety may include three optionally different heteroatoms (e.g., O, N, S, Si, Sn, or P). A heteroalkyl moiety may include four optionally different heteroatoms (e.g., O, N, S, Si, Sn, or P). A heteroalkyl moiety may include five optionally different heteroatoms (e.g., O, N, S, Si, Sn, or P). A heteroalkyl moiety may include up to 8 optionally different heteroatoms (e.g., O, N, S, Si, Sn, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂), where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocycloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

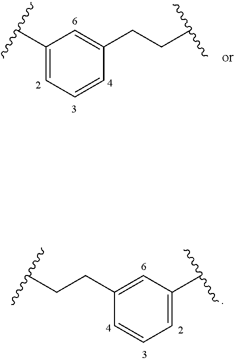 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR′N′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″N′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)₅—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), tin (Sn), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —S, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₅ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl, heteroalkyl, cycloalkyl, heterocycloalkyl, aryl, heteroaryl, substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCH₂Cl, —OCH₂Br, —OCH₂I, —OCH₂F, —N₃, —OCHF₂, unsubstituted alkyl (e.g., C₁-C₅ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl, heteroalkyl, cycloalkyl, heterocycloalkyl, aryl, heteroaryl, substituted with at least one substituent selected from:
      (a) oxo, halogen, —CCl₃, —CBr₃, —CF₃, —CI₃, —CHC₂, —CHBr₂, —CHF₂, —CHI₂, —CH₂Cl, —CH₂Br, —CH₂F, —CH₂I, —CN, —O, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₅ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl, heteroalkyl, cycloalkyl, heterocycloalkyl, aryl, heteroaryl, substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₅ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₅ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₅ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₅ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R³ substituents are present, each R³⁰ substituent may be distinguished as R^{30A}, R^{30B}, R^{30C}, R^{30D}, etc., wherein each of R^{30A}, R^{30B}, R^{30C}, R^{30D}, etc. is defined within the scope of the definition of R³⁰ and optionally differently.

A “detectable agent” or “detectable moiety” is a composition detectable by appropriate means such as spectroscopic, photochemical, biochemical, immunochemical, chemical, magnetic resonance imaging, or other physical means. For example, useful detectable agents include F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹¹¹Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, propionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present invention by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation”, “activate”, “activating”, “activator” and the like in reference to a protein-inhibitor interaction means positively affecting (e.g. increasing) the activity or function of the protein relative to the activity or function of the protein in the absence of the activator. In embodiments activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein.

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g., decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

In this disclosure, “comprises,” “comprising,” “containing” and “having” and the like can have the meaning ascribed to them in U.S. patent law and can mean “includes,” “including,” and the like. “Consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

The terms “disease” or “condition” refer to a state of being or health status of a patient or subject capable of being treated with the compounds or methods provided herein. The disease may be an infectious disease (e.g., a disease caused by a bacterial infection, a disease caused by a viral infection). In embodiments, the disease is a vancomycin-resistant Staphylococcus aureus (VRSA) infection. In embodiments, the disease is a vancomycin-resistant Enterococcus (VRE) infection.

The terms “treating”, or “treatment” refers to any indicia of success in the therapy or amelioration of an injury, disease, pathology or condition, including any objective or subjective parameter such as abatement (e.g., reducing fever, reducing diarrhea); remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment.

The term “prevent” refers to a decrease in the occurrence of disease symptoms (e.g., infectious disease symptoms) in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, pigs, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal) compatible with the preparation. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered transdermally, by a topical route, or formulated as applicator sticks, solutions, suspensions, emulsions, gels, creams, ointments, pastes, jellies, paints, powders, and aerosols. In embodiments, co-administer includes simultaneous or sequential administration of the compounds (e.g., compounds described herein) individually in addition to an additional secondary agent (e.g., additional antibiotic).

As used herein, the term “bioconjugate reactive moiety” and “bioconjugate” refers to the resulting association between atoms or molecules of bioconjugate reactive groups. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, olefin metathesis, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding; and
(m) metal bonding to reactive phosphorus groups (e.g. phosphines) to form, for example, phosphate diester bonds.
(n) azides coupled to alkynes using copper catalyzed cycloaddition click chemistry.
(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

The term “modulator” refers to a composition that increases or decreases the level of a target molecule or the function of a target molecule or the physical state of the target of the molecule. In some embodiments, a bacteria associated disease modulator is a compound that reduces the severity of one or more symptoms of a disease associated with bacterial infection.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g. a disease associated with bacterial infection activity) means that the disease (e.g. infectious disease) is caused by (in whole or in part), or a symptom of the disease is caused by (in whole or in part) the substance or substance activity or function. In embodiments, the infectious disease is a bacteria associated disease (e.g., actinomycosis, anthrax, abscesses in tissues (e.g., mouth in gastrointestinal tract, pelvic cavity, or lungs), whooping cough, lyme disease, brucellosis, enteritis, Guillain-Barre syndrome, pneumonia, conjunctivitis, trachoma, botulism, pseudomembranous colitis, food poisoning, tetanus, diphtheria, ehrlichiosis, bacterial endocarditis, urinary tract infection, diarrhea, meningitis (e.g., bacterial meningitis), sepsis, fever, tularemia, bronchitis, peptic ulcer, gastritis, Legionnaire's disease, Pontiac fever, leptospirosis, listeriosis, leprosy, gonorrhea, opthalmia, nocardiosis, typhoid fever, salmonellosis, shigellosis, impetigo, cystitis, Scarlet fever, syphilis, cholera, or plague.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

A “therapeutic agent” as used herein refers to an agent (e.g., compound or composition described herein) that when administered to a subject will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms or the intended therapeutic effect, e.g., treatment or amelioration of an injury, disease, pathology or condition, or their symptoms including any objective or subjective parameter of treatment such as abatement; remission; diminishing of symptoms or making the injury, pathology or condition more tolerable to the patient; slowing in the rate of degeneration or decline; making the final point of degeneration less debilitating; or improving a patient's physical or mental well-being.

The terms “immune response” and the like refer, in the usual and customary sense, to a response by an organism that protects against disease. The response can be mounted by the innate immune system or by the adaptive immune system, as well known in the art.

The terms “modulating immune response” and the like refer to a change in the immune response of a subject as a consequence of administration of an agent, e.g., a compound as disclosed herein, including embodiments thereof. Accordingly, an immune response can be activated or deactivated as a consequence of administration of an agent, e.g., a compound as disclosed herein, including embodiments thereof.

The term “leaving group” is used in accordance with its ordinary meaning in chemistry and refers to a moiety (e.g., atom, functional group, molecule) that separates from the molecule following a chemical reaction (e.g., bond formation, reductive elimination, condensation, cross-coupling reaction) involving an atom or chemical moiety to which the leaving group is attached, also referred to herein as the “leaving group reactive moiety”, and a complementary reactive moiety (i.e. a chemical moiety that reacts with the leaving group reactive moiety) to form a new bond between the remnants of the leaving groups reactive moiety and the complementary reactive moiety. Thus, the leaving group reactive moiety and the complementary reactive moiety form a complementary reactive group pair. Non limiting examples of leaving groups include hydrogen, hydroxide, organotin moieties (e.g., organotin heteroalkyl), halogen (e.g., Br), perfluoroalkylsulfonates (e.g. triflate), tosylates, mesylates, water, alcohols, nitrate, phosphate, thioether, amines, ammonia, fluoride, carboxylate, phenoxides, boronic acid, boronate esters, substituted or unsubstituted piperazinyl, and alkoxides. In embodiments, two molecules with leaving groups are allowed to contact, and upon a reaction and/or bond formation (e.g., acyloin condensation, aldol condensation, Claisen condensation, Stille reaction) the leaving groups separates from the respective molecule. In embodiments, a leaving group is a bioconjugate reactive moiety. In embodiments, at least two leaving groups (e.g., R¹ and R¹³) are allowed to contact such that the leaving groups are sufficiently proximal to react, interact or physically touch. In embodiments, the leaving groups is designed to facilitate the reaction. In embodiments, the leaving group is a substituent group.

The term “protecting group” is used in accordance with its ordinary meaning in organic chemistry and refers to a moiety covalently bound to a heteroatom, heterocycloalkyl, or heteroaryl to prevent reactivity of the heteroatom, heterocycloalkyl, or heteroaryl during one or more chemical reactions performed prior to removal of the protecting group. Typically a protecting group is bound to a heteroatom (e.g., O) during a part of a multipart synthesis wherein it is not desired to have the heteroatom react (e.g., a chemical reduction) with the reagent. Following protection the protecting group may be removed (e.g., by modulating the pH). In embodiments the protecting group is an alcohol protecting group. Non-limiting examples of alcohol protecting groups include acetyl, benzoyl, benzyl, methoxymethyl ether (MOM), tetrahydropyranyl (THP), and silyl ether (e.g., trimethylsilyl (TMS)). In embodiments the protecting group is an amine protecting group. Non-limiting examples of amine protecting groups include carbobenzyloxy (Cbz), tert-butyloxycarbonyl (BOC), 9-Fluorenylmethyloxycarbonyl (FMOC), acetyl, benzoyl, benzyl, carbamate, p-methoxybenzyl ether (PMB), and tosyl (Ts). In embodiments, the protecting group is —PO₃H or —SO₃H. In embodiments, the protecting group is a substituent group.

The term “infection” or “infectious disease” refers to a disease or condition that can be caused by organisms such as a bacterium, virus, fungi or any other pathogenic microbial agents. In embodiments, the infectious disease is caused by a pathogenic bacteria. Pathogenic bacteria are bacteria which cause diseases (e.g., in humans). In embodiments, the infectious disease is a bacteria associated disease (e.g., tuberculosis, which is caused by Mycobacterium tuberculosis). Non-limiting bacteria associated diseases include pneumonia, which may be caused by bacteria such as Streptococcus and Pseudomonas; or foodborne illnesses, which can be caused by bacteria such as Shigella, Campylobacter, and Salmonella. Bacteria associated diseases also includes tetanus, typhoid fever, diphtheria, syphilis, and leprosy. In embodiments, the disease is Bacterial vaginosis (i.e. bacteria that change the vaginal microbiota caused by an overgrowth of bacteria that crowd out the Lactobacilli species that maintain healthy vaginal microbial populations) (e.g., yeast infection, or Trichomonas vaginalis); Bacterial meningitis (i.e. a bacterial inflammation of the meninges); Bacterial pneumonia (i.e. a bacterial infection of the lungs); Urinary tract infection; Bacterial gastroenteritis; or Bacterial skin infections (e.g. impetigo, or cellulitis). In embodiments, the infectious disease is a Campylobacter jejuni, Enterococcus faecalis, Haemophilus influenzae, Helicobacter pylori, Klebsiella pneumoniae, Legionella pneumophila, Neisseria gonorrhoeae, Neisseria meningitides, Staphylococcus aureus, Streptococcus pneumonia, or Vibrio cholera infection.

The term “group A streptococcal infection” is an infection with group A streptococcus (GAS). Group A streptococcus refer to species of Gram-positive bacteria, non-motile and non-sporing cocci, which express the Lancefield group A antigen. Non-limiting examples of GAS include Streptococcus pyogenes, Streptococcus dysgalactiae, and Streptococcus anginosus. GAS typically colonize the throat, genital mucosa, rectum, and skin. Such bacteria can cause a variety of diseases such as streptococcal pharyngitis, rheumatic fever, rheumatic heart disease, and scarlet fever.

The term “Gram-positive bacteria” is used in accordance with its plain ordinary meaning and refers to bacteria that give a positive result in the Gram stain test. For example, Gram-positive bacteria take up the stain used in the test, and then appear to be purple-coloured when seen through a microscope due to the peptidoglycan layer in the bacterial cell wall. In contrast, Gram-negative bacteria cannot retain the violet stain.

The term “bacteriostatic agent” is used in accordance with its ordinary meaning and refers to a compound (e.g., a compound described herein) that inhibits bacteria reproduction. In embodiments, the bacteriostatic agent does not kill the bacterial cell. In embodiments, when a compound (e.g., a compound described herein) is administered to treat an infectious disease and it does not kill the bacteria cells but inhibits (e.g., reduces reproduction, ceases reproduction) bacteria cell reproduction, it may be considered a bacteriostatic agent.

### II. Compounds

In an aspect is provided a compound, or salt thereof, having the formula:

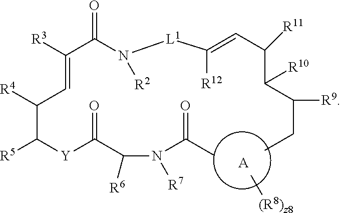

Y is —O— or —NH—. L¹ is a bond, substituted or unsubstituted alkylene, or substituted or unsubstituted heteroalkylene. R² is hydrogen or unsubstituted C₁-C₃ alkyl. R³, R⁴, and R⁵ are independently hydrogen, oxo, halogen, —CCl₃, —CBr₃, —CF₃, —CI₃, CHC₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃, —SO₄, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OC₃, —OCHCl₂, —OCHBr₂, —OCHI₂, —OCHF₂, —OCH₂Cl, —OCH₂Br, —OCH₂I, —OCH₂F, —N₃, —OPO₃H, —OSO₃H, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶ is hydrogen, halogen, —CCl₃, —CBr₃, —CF₃, —C₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or an amino acid side chain. R⁷ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —CCl₃, —COOH, —CH₂COOH, —CONH₂, —OH, —SH, —NO₂, —NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶ and R⁷ may optionally be joined to form a substituted or unsubstituted heterocycloalkyl or substituted or unsubstituted heteroaryl. R′ is oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Ring A is cycloalkylene, heterocycloalkylene, arylene, or heteroarylene. The symbol z8 is an integer from 0 to 10. R⁹, R¹⁰, R¹¹, and R¹² are each independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COO, —CONH₂, —NO₂, —SH, —SO₃, —SO₄, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, the compound does not have the formula:

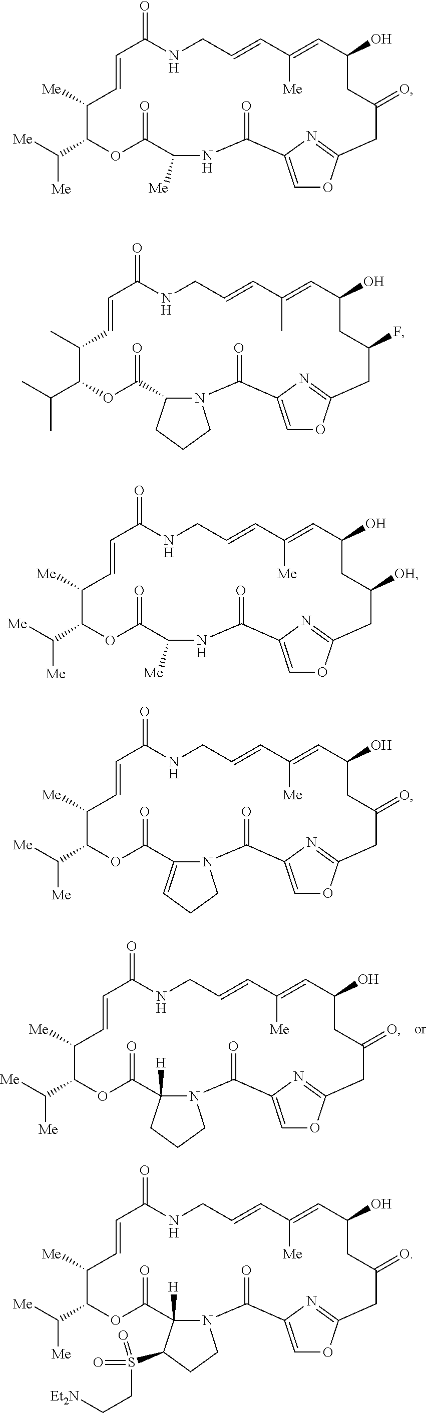

In an aspect is provided a compound, or salt thereof, having the formula:

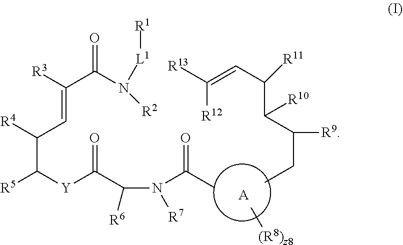

R¹ and R¹³ are leaving groups. Y is —O— or —NH—. L¹ is a bond, substituted or unsubstituted alkylene, or substituted or unsubstituted heteroalkylene. R² is hydrogen or unsubstituted C₁-C₃ alkyl. R³, R⁴, and R⁵ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —S₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —OPO₃H, —OSO₃H, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or an amino acid side chain. R⁷ is hydrogen, halogen, —CCl₃, —CBr₃, —CF₃, —CI₃, CHC₂, —CHBr₂, —CHF₂, —CHI₂, —CH₂Cl, —CH₂Br, —CH₂F, —CH₂I, —CN, —CCl₃, —COO, —CH₂COOH, —CONH₂, —O, —SH, —NO₂, —NH2, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶ and R⁷ may optionally be joined to form a substituted or unsubstituted heterocycloalkyl or substituted or unsubstituted heteroaryl. R⁸ is oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Ring A is cycloalkylene, heterocycloalkylene, arylene, or heteroarylene. The symbol z8 is an integer from 0 to 10. R⁹, R¹⁰, R¹¹, and R¹² are each independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COO, —CONH₂, —NO₂, —SH, —SO₃, —SO₄, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OC₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In an aspect is provided a compound, or salt thereof, having the formula:

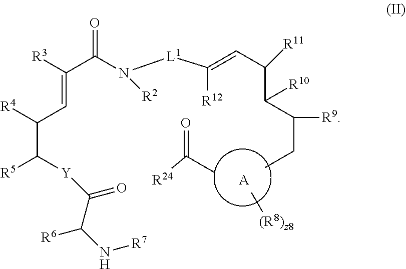

R²⁴ is a leaving group. Y is —O— or —NH—. L¹ is a bond, substituted or unsubstituted alkylene, or substituted or unsubstituted heteroalkylene. R² is hydrogen or unsubstituted C₁-C₃ alkyl. R³, R⁴, and R⁵ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃, —SO₄, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —OPO₃H, —OSO₃H, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OC₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or an amino acid side chain. R⁷ is hydrogen, halogen, —CCl₃, —CBr₃, —CF₃, —CI₃, CHC₂, —CHBr₂, —CHF₂, —CHI₂, —CH₂Cl, —CH₂Br, —CH₂F, —CH₂I, —CN, —CCl₃, —COOH, —CH₂COOH, —CONH₂, —OH, —SH, —NO₂, —NH2, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶ and R⁷ may optionally be joined to form a substituted or unsubstituted heterocycloalkyl or substituted or unsubstituted heteroaryl. R⁸ is oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Ring A is cycloalkylene, heterocycloalkylene, arylene, or heteroarylene. The symbol z8 is an integer from 0 to 10. R⁹, R¹⁰, R¹¹, and R¹² are each independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COO, —CONH₂, —NO₂, —SH, —SO₃, —SO₄, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In another aspect is provided a compound, or salt thereof, having the formula:

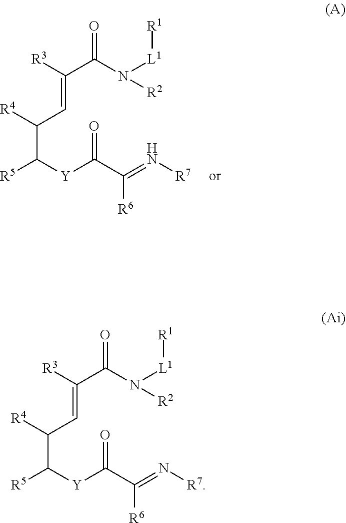

R¹ is a leaving group. Y is —O— or —NH—. L¹ is a bond, substituted or unsubstituted alkylene, or substituted or unsubstituted heteroalkylene. R² is hydrogen or unsubstituted C₁-C₃ alkyl. R³, R⁴, and R⁵ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃, —SO₄, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —OPO₃H, —OSO₃H, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or an amino acid side chain. R⁷ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —CCl₃, —COOH, —CH₂COOH, —CONH₂, —OH, —SH, —NO₂, —NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R⁶ and R⁷ may optionally be joined to form a substituted or unsubstituted heterocycloalkyl or substituted or unsubstituted heteroaryl.

In an aspect is provided a compound, or salt thereof, having the formula:

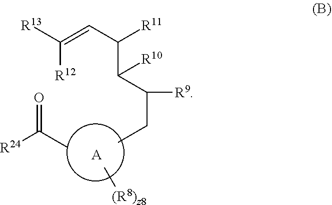

R¹³ and R²⁴ are leaving groups. R⁸ is oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Ring A is cycloalkyl, heterocycloalkyl, aryl, or heteroaryl. The symbol z8 is an integer from 0 to 10. R⁹, R¹⁰, R¹¹, and R¹ are each independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COO, —CONH₂, —NO₂, —SH, —SO₃, —SO₄, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In an aspect is provided a compound, or salt thereof, having the formula:

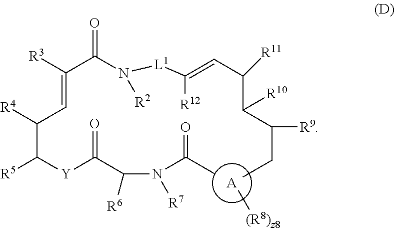

Y is —O— or —NH—. L¹ is a bond, substituted or unsubstituted alkylene, or substituted or unsubstituted heteroalkylene. R² is hydrogen or unsubstituted C₁-C₃ alkyl. R³, R⁴, and R⁵ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃, —SO₄, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —OPO₃H, —OSO₃H, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or an amino acid side chain. R⁷ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —CCl₃, —COOH, —CH₂COOH, —CONH₂, —OH, —SH, —NO₂, —NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹ and R⁷ may optionally be joined to forma substituted or unsubstituted heterocycloalkyl or substituted or unsubstituted heteroaryl. R⁸ is oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCI₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Ring A is cycloalkyl, heterocycloalkyl, aryl, or heteroaryl. The symbol z8 is an integer from 0 to 10. R⁹ is hydrogen, oxo, halogen, —CN, —OH, —NH₂, —COOH, —CONH₂, —NO₂, —SH, —SO₃H, —N₃, unsubstituted alkyl, or unsubstituted heteroalkyl. R¹⁰ and R¹² are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹¹ is hydrogen, oxo, halogen, —CCl₃, —CBr₃, —CF₃, —C₃, CHC₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OC₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, the compound does not have the formula:

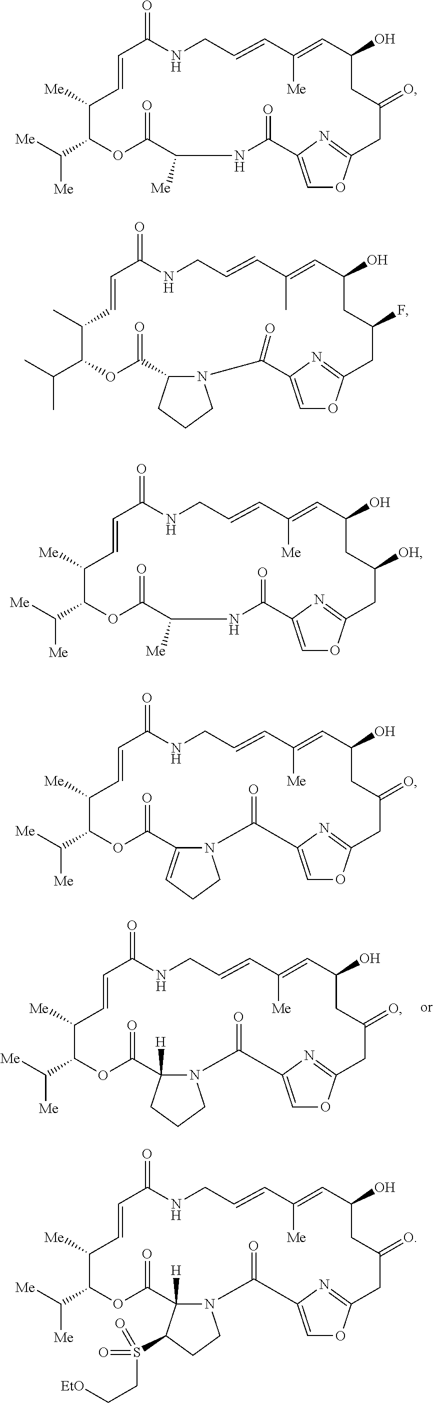

In embodiments, Y is —O—. In embodiments, Y is —NH—. In embodiments, Y¹ is ═O. In embodiments, Y¹ is ═S. In embodiments, Y² is —O—. In embodiments, Y² is —S—. In embodiments, Y⁴ is ═O. In embodiments, Y⁴ is ═S. In embodiments, Y⁵ is —O—. In embodiments, Y is —S—. In embodiments, Y³ is —S—, —O—, or —NR^{8.4}-. In embodiments, Y³ is —S—. In embodiments, Y³ is —O—. In embodiments, Y³ is —NR^{8.4}-. In embodiments, Y³ is —NH—.

In embodiments, L¹ is a substituted or unsubstituted alkylene, substituted or unsubstituted heteroalkylene. In embodiments, L¹ is a bond. In embodiments, L¹ is a substituted or unsubstituted alkylene. In embodiments, L¹ is a substituted or unsubstituted alkenylene. In embodiments, L¹ is a substituted or unsubstituted heteroalkylene.

In embodiments, L¹ is substituted or unsubstituted alkylene (e.g., C₁-C₈ alkylene, C₁-C₆ alkylene, or C₁-C₄ alkylene). In embodiments, L¹ is substituted alkylene (e.g., C₁-C₈ alkylene, C₁-C₆ alkylene, or C₁-C₄ alkylene). In embodiments, L¹ is an unsubstituted alkylene (e.g., C₁-C₈ alkylene, C₁-C₆ alkylene, or C₁-C₄ alkylene). In embodiments, L¹ is an unsubstituted C₁-C₈ alkylene. In embodiments, L¹ is an unsubstituted C₂-C₆ alkylene. In embodiments, L¹ is an unsubstituted C₂-C₄ alkylene. In embodiments, L¹ is an unsubstituted C₂-C₃ alkylene.

In embodiments, L¹ is substituted or unsubstituted alkenylene (e.g., C₁-C₅ alkenylene, C₁-C₆ alkenylene, or C₁-C₄ alkenylene). In embodiments, L¹ is substituted alkenylene (e.g., C₁-C₅ alkenylene, C₁-C₆ alkenylene, or C₁-C₄ alkenylene). In embodiments, L¹ is an unsubstituted alkenylene (e.g., C₁-C₅ alkenylene, C₁-C₆ alkenylene, or C₁-C₄ alkenylene). In embodiments, L¹ is an unsubstituted C₁-C₅ alkenylene. In embodiments, L¹ is an unsubstituted C₂-C₆ alkenylene. In embodiments, L¹ is an unsubstituted C₂-C₄ alkenylene. In embodiments, L¹ is an unsubstituted C₂-C₃ alkenylene.

In embodiments, L¹ is substituted or unsubstituted heteroalkylene (e.g., 2 to 8 membered heteroalkylene, 2 to 6 membered heteroalkylene, or 2 to 4 membered heteroalkylene). In embodiments, L¹ is substituted heteroalkylene (e.g., 2 to 8 membered heteroalkylene, 2 to 6 membered heteroalkylene, or 2 to 4 membered heteroalkylene). In embodiments, L¹ is an unsubstituted heteroalkylene (e.g., 2 to 8 membered heteroalkylene, 2 to 6 membered heteroalkylene, or 2 to 4 membered heteroalkylene).

In embodiments, L¹ is a substituted or unsubstituted alkylene, substituted or unsubstituted heteroalkylene. In embodiments, L¹ is a substituted or unsubstituted alkenylene. In embodiments, L¹ is a substituted or unsubstituted C₁-C₃ alkenylene. In embodiments, L¹ has the formula:

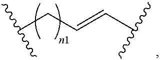

wherein n1 is an integer from 0 to 10. In embodiments, n1 is 0. In embodiments, n1 is 1. In embodiments, n1 is 2. In embodiments, n1 is 3. In embodiments, L¹ is

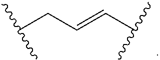 [SMILES: */C=C/C*]

In embodiments, L¹ is

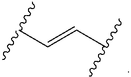 [SMILES: */C=C/*]

In embodiments, L¹ is a bond, R⁶¹-substituted or unsubstituted alkylene (e.g., C₁-C₅ alkylene, C₁-C₆ alkylene, or C₁-C₄ alkylene), or R⁶¹-substituted or unsubstituted heteroalkylene (e.g., 2 to 8 membered heteroalkylene, 2 to 6 membered heteroalkylene, or 2 to 4 membered heteroalkylene).

In embodiments, L¹ is R⁶¹-substituted or unsubstituted alkylene (e.g., C₁-C₅ alkylene, C₁-C₆ alkylene, or C₁-C₄ alkylene). In embodiments, L¹ is R⁶¹-substituted alkylene (e.g., C₁-C₅ alkylene, C₁-C₆ alkylene, or C₁-C₄ alkylene). In embodiments, L¹ is an unsubstituted alkylene (e.g., C₁-C₅ alkylene, C₁-C₆ alkylene, or C₁-C₄ alkylene).

In embodiments, L¹ is R⁶¹-substituted or unsubstituted heteroalkylene (e.g., 2 to 8 membered heteroalkylene, 2 to 6 membered heteroalkylene, or 2 to 4 membered heteroalkylene). In embodiments, L¹ is R⁶¹-substituted heteroalkylene (e.g., 2 to 8 membered heteroalkylene, 2 to 6 membered heteroalkylene, or 2 to 4 membered heteroalkylene). In embodiments, L¹ is an unsubstituted heteroalkylene (e.g., 2 to 8 membered heteroalkylene, 2 to 6 membered heteroalkylene, or 2 to 4 membered heteroalkylene).

In embodiments, the atom or chemical moieties to which R¹ and R are attached to a complementary reactive group pair (e.g., bioconjugate reactive moieties which form a bioconjugate linker). For example, L¹ and the carbon to which R¹³ is attached may be complementary reactive group pairs. For example, the atom (e.g., carbon) adjacent to R¹ on L¹ and the carbon to which R¹³ is attached may be complementary reactive group pairs which form a bioconjugate linker. In embodiments, R and R are bioconjugate reactive moieties. In embodiments, R¹ and R¹³ are allowed to contact such that R¹ and R¹³ are sufficiently proximal to react, interact or physically touch. In embodiments, R¹ and R¹³ are leaving groups following a reaction (e.g., a cross coupling reaction).

In embodiments, R¹ and R¹³ react to form a covalent linker. In embodiments, R¹ and R¹³ react to form an unsubstituted alkyl. In embodiments, R¹ and R¹³ react to form an unsubstituted alkenyl. In embodiments, R¹ is an unsubstituted C₂ alkenyl. In embodiments, R¹ is an unsubstituted C₃ alkenyl. In embodiments, R¹³ is an unsubstituted C₂ alkenyl. In embodiments, R¹³ is an unsubstituted C₃ alkenyl.

In embodiments, R¹ is hydrogen, halogen, substituted or unsubstituted amine, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹ is hydrogen or R⁵⁰-substituted or unsubstituted alkyl (e.g., C₁-C₅ alkyl, C₁-C₆ alkyl, or C₁-C₄ alkyl), R⁵⁰-substituted or unsubstituted heteroalkyl (e.g., 2 to 8 membered heteroalkyl, 2 to 6 membered heteroalkyl, or 2 to 4 membered heteroalkyl), R⁵⁰-substituted or unsubstituted cycloalkyl (e.g., C₃-C₈ cycloalkyl, C₃-C₆ cycloalkyl, or C₅-C₆ cycloalkyl), R⁵⁰-substituted or unsubstituted heterocycloalkyl (e.g., 3 to 8 membered heterocycloalkyl, 3 to 6 membered heterocycloalkyl, or 5 to 6 membered heterocycloalkyl), R⁵⁰-substituted or unsubstituted aryl (e.g., C₆-C₁₀ aryl, C₁₀ aryl, or phenyl), or R⁵⁰-substituted or unsubstituted heteroaryl (e.g., 5 to 10 membered heteroaryl, 5 to 9 membered heteroaryl, or 5 to 6 membered heteroaryl).

In embodiments, R¹ is R⁵⁰-substituted or unsubstituted alkyl (e.g., C₁-C₅ alkyl, C₁-C₆ alkyl, or C₁-C₄ alkyl). In embodiments, R¹ is R⁵⁰-substituted alkyl (e.g., C₁-C₅ alkyl, C₁-C₆ alkyl, or C₁-C₄ alkyl). In embodiments, R¹ is an unsubstituted alkyl (e.g., C₁-C₅ alkyl, C₁-C₆ alkyl, or C₁-C₄ alkyl).

In embodiments, R¹ is R⁵⁰-substituted or unsubstituted heteroalkyl (e.g., 2 to 8 membered heteroalkyl, 2 to 6 membered heteroalkyl, or 2 to 4 membered heteroalkyl). In embodiments, R¹ is R⁵⁰-substituted heteroalkyl (e.g., 2 to 8 membered heteroalkyl, 2 to 6 membered heteroalkyl, or 2 to 4 membered heteroalkyl). In embodiments, R¹ is an unsubstituted heteroalkyl (e.g., 2 to 8 membered heteroalkyl, 2 to 6 membered heteroalkyl, or 2 to 4 membered heteroalkyl).

In embodiments, R¹ is R⁵⁰-substituted or unsubstituted cycloalkyl (e.g., C₃-C₈ cycloalkyl, C₃-C₆ cycloalkyl, or C₅-C₆ cycloalkyl). In embodiments, R¹ is R⁵⁰-substituted cycloalkyl (e.g., C₃-C₈ cycloalkyl, C₃-C₆ cycloalkyl, or C₅-C₆ cycloalkyl). In embodiments, R¹ is an unsubstituted cycloalkyl (e.g., C₃-C₈ cycloalkyl, C₃-C₆ cycloalkyl, or C₅-C₆ cycloalkyl).

In embodiments, R¹ is R⁵⁰-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R¹ is R⁵⁰-substituted heterocycloalkyl (e.g., 3 to 8 membered heterocycloalkyl, 3 to 6 membered heterocycloalkyl, or 5 to 6 membered heterocycloalkyl). In embodiments, R¹ is an unsubstituted heterocycloalkyl (e.g., 3 to 8 membered heterocycloalkyl, 3 to 6 membered heterocycloalkyl, or 5 to 6 membered heterocycloalkyl).

In embodiments, R¹ is R⁵⁰-substituted or unsubstituted aryl (e.g., C₆-C₁₀ aryl, C₁₀ aryl, or phenyl). In embodiments, R¹ is R⁵⁰-substituted aryl (e.g., C₆-C₁₀ aryl, C₁₀ aryl, or phenyl). In embodiments, R¹ is an unsubstituted aryl (e.g., C₆-C₁₀ aryl, C₁₀ aryl, or phenyl).

In embodiments, R¹ is R⁵⁰-substituted or unsubstituted heteroaryl (e.g., 5 to 10 membered heteroaryl, 5 to 9 membered heteroaryl, or 5 to 6 membered heteroaryl). In embodiments, R¹ is R⁵⁰-substituted heteroaryl (e.g., 5 to 10 membered heteroaryl, 5 to 9 membered heteroaryl, or 5 to 6 membered heteroaryl). In embodiments, R¹ is an unsubstituted heteroaryl (e.g., 5 to 10 membered heteroaryl, 5 to 9 membered heteroaryl, or 5 to 6 membered heteroaryl).

R³ is independently oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R¹ is an organotin heteroalkyl, having the formula:

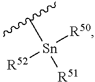

wherein R⁵⁰, R⁵¹, and R⁵² are independently unsubstituted alkyl (e.g., C₁-C₈ alkyl, C₁-C₆ alkyl, or C₁-C₄ alkyl) or unsubstituted aryl (e.g., C₆-C₁₀ aryl, C₁₀ aryl, or phenyl).

In embodiments, R¹ is

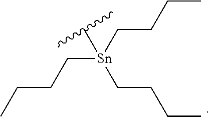 [SMILES: [*][Sn]([CH2]CCC)([CH2]CCC)[CH2]CCC]

In embodiments, R¹ is a halogen. In embodiments, R¹ is —Br. In embodiments, R¹ is hydrogen.

In embodiments, R¹ is a carboxylic acid and R″ is a substituted or unsubstituted amine (e.g., —NHCH₃, —NH₂).

In embodiments, R² is hydrogen or unsubstituted methyl. In embodiments, R² is hydrogen. In embodiments, R² is an unsubstituted C₁-C₃ alkyl. In embodiments, R² is an unsubstituted methyl. In embodiments, R² is an unsubstituted C₂ alkyl. In embodiments, R² is an unsubstituted C₃ alkyl. In embodiments, R² is an unsubstituted C₄ alkyl. In embodiments, R² is an unsubstituted C₅ alkyl. In embodiments, R² is an unsubstituted C₆ alkyl. In embodiments, R² is an unsubstituted C₇ alkyl. In embodiments, R² is an unsubstituted C₅ alkyl.

In embodiments, R³ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, or —N₃. In embodiments, R³ is hydrogen. In embodiments, R³ is oxo. In embodiments, R³ is halogen. In embodiments, R³ is —CCl₃. In embodiments, R³ is —CBr₃. In embodiments, R³ is —CF₃. In embodiments, R³ is —CI₃. In embodiments, R³ is CHCl₂. In embodiments, R³ is —CHBr₂. In embodiments, R³ is —CHF₂. In embodiments, R³ is —CHI₂. In embodiments, R³ is —CH₂Cl. In embodiments, R³ is —CH₂Br. In embodiments, R³ is —CH₂F. In embodiments, R³ is —CH₂I. In embodiments, R³ is —CN. In embodiments, R³ is —OH. In embodiments, R³ is —NH₂. In embodiments, R³ is —COOH. In embodiments, R³ is —CONH₂. In embodiments, R³ is —NO₂. In embodiments, R³ is —SH. In embodiments, R³ is —SO₃H. In embodiments, R³ is —SO₄H. In embodiments, R³ is —SO₂NH₂. In embodiments, R³ is —NHNH₂. In embodiments, R³ is —ONH₂. In embodiments, R³ is —NHC(O)NHNH₂. In embodiments, R³ is —NHC(O)NH₂. In embodiments, R³ is —NHSO₂H. In embodiments, R³ is —NHC(O)H. In embodiments, R³ is —NHC(O)OH. In embodiments, R³ is —NHOH. In embodiments, R³ is —OCCl₃. In embodiments, R³ is —OCF₃. In embodiments, R³ is —OCBr₃. In embodiments, R³ is —OCI₃. In embodiments, R³ is —OCHCl₂. In embodiments, R³ is —OCHBr₂. In embodiments, R³ is —OCHI₂. In embodiments, R³ is —OCHF₂. In embodiments, R³ is —OCH₂Cl. In embodiments, R³ is —OCH₂Br. In embodiments, R³ is —OCH₂I. In embodiments, R³ is —OCH₂F. In embodiments, R³ is —N₃. In embodiments, R³ is hydrogen or substituted or unsubstituted alkyl. In embodiments, R³ is —OPO₃H. In embodiments, R³ is —OSO₃H.

In embodiments, R³ is a substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R³ is substituted (e.g., substituted with a substituent group, a size-limited substituent group, or lower substituent group) or unsubstituted alkyl. In embodiments, R³ is substituted (e.g., substituted with a substituent group, a size-limited substituent group, or lower substituent group) alkyl. In embodiments, R³ is unsubstituted alkyl. In embodiments, R³ is substituted or unsubstituted alkyl (e.g., C₁-C₅, C₁-C₆, C₁-C₄, or C₁-C₂). In embodiments, R³ is substituted alkyl (e.g., C₁-C₈, C₁-C₆, C₁-C₄, or C₁-C₂). In embodiments, R³ is unsubstituted alkyl (e.g., C₁-C₈, C₁-C₆, C₁-C₄, or C₁-C₂).

In embodiments, R³ is substituted (e.g., substituted with a substituent group, a size-limited substituent group, or lower substituent group) or unsubstituted heteroalkyl. In embodiments, R³ is substituted (e.g., substituted with a substituent group, a size-limited substituent group, or lower substituent group) heteroalkyl. In embodiments, R³ is unsubstituted heteroalkyl. In embodiments, R³ is substituted or unsubstituted heteroalkyl (e.g., 2 to 8 membered, 2 to 6 membered, 4 to 6 membered, 2 to 3 membered, or 4 to 5 membered). In embodiments, R³ is substituted heteroalkyl (e.g., 2 to 8 membered, 2 to 6 membered, 4 to 6 membered, 2 to 3 membered, or 4 to 5 membered). In embodiments, R³ is an unsubstituted heteroalkyl (e.g., 2 to 8 membered, 2 to 6 membered, 4 to 6 membered, 2 to 3 membered, or 4 to 5 membered).

In embodiments, R³ is substituted (e.g., substituted with a substituent group, a size-limited substituent group, or lower substituent group) or unsubstituted cycloalkyl. In embodiments, R³ is substituted (e.g., substituted with a substituent group, a size-limited substituent group, or lower substituent group) cycloalkyl. In embodiments, R³ is an unsubstituted cycloalkyl. In embodiments, R³ is substituted or unsubstituted cycloalkyl (e.g., C₃-C₈, C₃-C₆, C₄-C₆, or C₅-C₆). In embodiments, R³ is substituted cycloalkyl (e.g., C₃-C₈, C₃-C₆, C₄-C₆, or C₅-C₆). In embodiments, R³ is unsubstituted cycloalkyl (e.g., C₃-C₈, C₃-C₆, C₄-C₆, or C₅-C₆).

In embodiments, R³ is substituted (e.g., substituted with a substituent group, a size-limited substituent group, or lower substituent group) or unsubstituted heterocycloalkyl. In embodiments, R³ is substituted (e.g., substituted with a substituent group, a size-limited substituent group, or lower substituent group) heterocycloalkyl. In embodiments, R³ is an unsubstituted heterocycloalkyl. In embodiments, R³ is substituted or unsubstituted heterocycloalkyl (e.g., 3 to 8 membered, 3 to 6 membered, 4 to 6 membered, 4 to 5 membered, or 5 to 6 membered). In embodiments, R³ is substituted heterocycloalkyl (e.g., 3 to 8 membered, 3 to 6 membered, 4 to 6 membered, 4 to 5 membered, or 5 to 6 membered). In embodiments, R³ an unsubstituted heterocycloalkyl (e.g., 3 to 8 membered, 3 to 6 membered, 4 to 6 membered, 4 to 5 membered, or 5 to 6 membered).

In embodiments, R³ is substituted (e.g., substituted with a substituent group, a size-limited substituent group, or lower substituent group) or unsubstituted aryl. In embodiments, R³ is substituted (e.g., substituted with a substituent group, a size-limited substituent group, or lower substituent group) aryl. In embodiments, R³ is an unsubstituted aryl. In embodiments, R³ is substituted or unsubstituted aryl (e.g., C₆-C₁₀ or phenyl). In embodiments, R³ is substituted aryl (e.g., C₆-C₁₀ or phenyl). In embodiments, R³ is an unsubstituted aryl (e.g., C₆-C₁₀ or phenyl).

In embodiments, R³ is substituted (e.g., substituted with a substituent group, a size-limited substituent group, or lower substituent group) or unsubstituted heteroaryl. In embodiments, R³ is substituted (e.g., substituted with a substituent group, a size-limited substituent group, or lower substituent group) heteroaryl. In embodiments, R³ is an unsubstituted heteroaryl. In embodiments, R³ is substituted or unsubstituted heteroaryl (e.g., 5 to 10 membered, 5 to 9 membered, or 5 to 6 membered). In embodiments, R³ is substituted heteroaryl (e.g., 5 to 10 membered, 5 to 9 membered, or 5 to 6 membered). In embodiments, R³ is an unsubstituted heteroaryl (e.g., 5 to 10 membered, 5 to 9 membered, or 5 to 6 membered).

In embodiments, R³ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R³ is hydrogen, substituted or unsubstituted cycloalkyl, substituted or unsubstituted heterocycloalkyl, substituted or unsubstituted aryl, or substituted or unsubstituted heteroaryl.

In embodiments, R³ is hydrogen or R³⁸-substituted or unsubstituted alkyl (e.g., C₁-C₅ alkyl, C₁-C₆ alkyl, or C₁-C₄ alkyl), R⁵⁰-substituted or unsubstituted heteroalkyl (e.g., 2 to 8 membered heteroalkyl, 2 to 6 membered heteroalkyl, or 2 to 4 membered heteroalkyl), R³⁸-substituted or unsubstituted cycloalkyl (e.g., C₃-C₈ cycloalkyl, C₃-C₆ cycloalkyl, or C₅-C₆ cycloalkyl), R³⁸-substituted or unsubstituted heterocycloalkyl (e.g., 3 to 8 membered heterocycloalkyl, 3 to 6 membered heterocycloalkyl, or 5 to 6 membered heterocycloalkyl), R³⁸-substituted or unsubstituted aryl (e.g., C₆-C₁₀ aryl, C₁₀ aryl, or phenyl), or R³⁸-substituted or unsubstituted heteroaryl (e.g., 5 to 10 membered heteroaryl, 5 to 9 membered heteroaryl, or 5 to 6 membered heteroaryl).

In embodiments, R³ is R³⁸-substituted or unsubstituted alkyl (e.g., C₁-C₅ alkyl, C₁-C₆ alkyl, or C₁-C₄ alkyl). In embodiments, R³ is R³⁸-substituted alkyl (e.g., C₁-C₅ alkyl, C₁-C₆ alkyl, or C₁-C₄ alkyl). In embodiments, R³ is an unsubstituted alkyl (e.g., C₁-C₅ alkyl, C₁-C₆ alkyl, or C₁-C₄ alkyl).

In embodiments, R³ is R³⁸-substituted or unsubstituted heteroalkyl (e.g., 2 to 8 membered heteroalkyl, 2 to 6 membered heteroalkyl, or 2 to 4 membered heteroalkyl). In embodiments, R³ is R³⁸-substituted heteroalkyl (e.g., 2 to 8 membered heteroalkyl, 2 to 6 membered heteroalkyl, or 2 to 4 membered heteroalkyl). In embodiments, R³ is an unsubstituted heteroalkyl (e.g., 2 to 8 membered heteroalkyl, 2 to 6 membered heteroalkyl, or 2 to 4 membered heteroalkyl).

In embodiments, R³ is R³⁸-substituted or unsubstituted cycloalkyl (e.g., C₃-C₈ cycloalkyl, C₃-C₆ cycloalkyl, or C₅-C₆ cycloalkyl). In embodiments, R³ is R³⁸-substituted cycloalkyl (e.g., C₃-C₈ cycloalkyl, C₃-C₆ cycloalkyl, or C₅-C₆ cycloalkyl). In embodiments, R³ is an unsubstituted cycloalkyl (e.g., C₃-C₈ cycloalkyl, C₃-C₆ cycloalkyl, or C₅-C₆ cycloalkyl).

In embodiments, R³ is R³⁸-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R³ is R³⁸-substituted heterocycloalkyl (e.g., 3 to 8 membered heterocycloalkyl, 3 to 6 membered heterocycloalkyl, or 5 to 6 membered heterocycloalkyl). In embodiments, R³ is an unsubstituted heterocycloalkyl (e.g., 3 to 8 membered heterocycloalkyl, 3 to 6 membered heterocycloalkyl, or 5 to 6 membered heterocycloalkyl).

In embodiments, R³ is R³⁸-substituted or unsubstituted aryl (e.g., C₆-C₁₀ aryl, C₁₀ aryl, or phenyl). In embodiments, R³ is R³⁸-substituted aryl (e.g., C₆-C₁₀ aryl, C₁₀ aryl, or phenyl). In embodiments, R³ is an unsubstituted aryl (e.g., C₆-C₁₀ aryl, C₁₀ aryl, or phenyl).

In embodiments, R³ is R³⁸-substituted or unsubstituted heteroaryl (e.g., 5 to 10 membered heteroaryl, 5 to 9 membered heteroaryl, or 5 to 6 membered heteroaryl). In embodiments, R³ is R³⁸-substituted heteroaryl (e.g., 5 to 10 membered heteroaryl, 5 to 9 membered heteroaryl, or 5 to 6 membered heteroaryl). In embodiments, R³ is an unsubstituted heteroaryl (e.g., 5 to 10 membered heteroaryl, 5 to 9 membered heteroaryl, or 5 to 6 membered heteroaryl).

In embodiments, R⁴ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, or —N₃. In embodiments, R⁴ is hydrogen. In embodiments, R⁴ is oxo. In embodiments, R⁴ is halogen. In embodiments, R⁴ is —CCl₃. In embodiments, R⁴ is —CBr₃. In embodiments, R⁴ is —CF₃. In embodiments, R⁴ is —CI₃. In embodiments, R⁴ is CHCl₂. In embodiments, R⁴ is —CHBr₂. In embodiments, R⁴ is —CHF₂. In embodiments, R⁴ is —CHI₂. In embodiments, R⁴ is —CH₂C₁. In embodiments, R⁴ is —CH₂Br. In embodiments, R⁴ is —CH₂F. In embodiments, R⁴ is —CH₂I. In embodiments, R⁴ is —CN. In embodiments, R⁴ is —OH. In embodiments, R⁴ is —NH₂. In embodiments, R⁴ is —COOH. In embodiments, R⁴ is —CONH₂. In embodiments, R⁴ is —NO₂. In embodiments, R⁴ is —SH. In embodiments, R⁴ is —SO₃H. In embodiments, R⁴ is —SO₄H. In embodiments, R⁴ is —SO₂NH₂. In embodiments, R⁴ is —NHNH₂. In embodiments, R⁴ is —ONH₂. In embodiments, R⁴ is —NHC(O)NHNH₂. In embodiments, R⁴ is —NHC(O)NH₂. In embodiments, R⁴ is —NHSO₂H. In embodiments, R⁴ is —NHC(O)H. In embodiments, R⁴ is —NHC(O)OH. In embodiments, R⁴ is —NHOH. In embodiments, R⁴ is —OCCl₃. In embodiments, R⁴ is —OCF₃. In embodiments, R⁴ is —OCBr₃. In embodiments, R⁴ is —OCI₃. In embodiments, R⁴ is —OCHCl₂. In embodiments, R⁴ is —OCHBr₂. In embodiments, R⁴ is —OCHI₂. In embodiments, R⁴ is —OCHF₂. In embodiments, R⁴ is —OCH₂Cl. In embodiments, R⁴ is —OCH₂Br. In embodiments, R⁴ is —OCH₂I. In embodiments, R⁴ is —OCH₂F. In embodiments, R⁴ is —N₃. In embodiments, R⁴ is hydrogen or substituted or unsubstituted alkyl. In embodiments, R⁴ is hydrogen or a substituted or unsubstituted C₁-C₆ alkyl. In embodiments, R⁴ is —OPO₃H. In embodiments, R⁴ is —OSO₃H.

In embodiments, R⁴ is a substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁴ is substituted (e.g., substituted with a substituent group, a size-limited substituent group, or lower substituent group) or unsubstituted alkyl. In embodiments, R⁴ is substituted (e.g., substituted with a substituent group, a size-limited substituent group, or lower substituent group) alkyl. In embodiments, R⁴ is unsubstituted alkyl. In embodiments, R⁴ is substituted or unsubstituted alkyl (e.g., C₁-C₅, C₁-C₆, C₁-C₄, or C₁-C₂). In embodiments, R⁴ is substituted alkyl (e.g., C₁-C₈, C₁-C₆, C₁-C₄, or C₁-C₂). In embodiments, R⁴ is unsubstituted alkyl (e.g., C₁-C₈, C₁-C₆, C₁-C₄, or C₁-C₂).

In embodiments, R⁴ is substituted (e.g., substituted with a substituent group, a size-limited substituent group, or lower substituent group) or unsubstituted heteroalkyl. In embodiments, R⁴ is substituted (e.g., substituted with a substituent group, a size-limited substituent group, or lower substituent group) heteroalkyl. In embodiments, R⁴ is unsubstituted heteroalkyl. In embodiments, R⁴ is substituted or unsubstituted heteroalkyl (e.g., 2 to 8 membered, 2 to 6 membered, 4 to 6 membered, 2 to 3 membered, or 4 to 5 membered). In embodiments, R⁴ is substituted heteroalkyl (e.g., 2 to 8 membered, 2 to 6 membered, 4 to 6 membered, 2 to 3 membered, or 4 to 5 membered). In embodiments, R⁴ is an unsubstituted heteroalkyl (e.g., 2 to 8 membered, 2 to 6 membered, 4 to 6 membered, 2 to 3 membered, or 4 to 5 membered).

In embodiments, R⁴ is substituted (e.g., substituted with a substituent group, a size-limited substituent group, or lower substituent group) or unsubstituted cycloalkyl. In embodiments, R⁴ is substituted (e.g., substituted with a substituent group, a size-limited substituent group, or lower substituent group) cycloalkyl. In embodiments, R⁴ is an unsubstituted cycloalkyl. In embodiments, R⁴ is substituted or unsubstituted cycloalkyl (e.g., C₃-C₈, C₃-C₆, C₄-C₆, or C₅-C₆). In embodiments, R⁴ is substituted cycloalkyl (e.g., C₃-C₈, C₃-C₆, C₄-C₆, or C₅-C₆). In embodiments, R⁴ is unsubstituted cycloalkyl (e.g., C₃-C₈, C₃-C₆, C₄-C₆, or C₅-C₆).

In embodiments, R⁴ is substituted (e.g., substituted with a substituent group, a size-limited substituent group, or lower substituent group) or unsubstituted heterocycloalkyl. In embodiments, R⁴ is substituted (e.g., substituted with a substituent group, a size-limited substituent group, or lower substituent group) heterocycloalkyl. In embodiments, R⁴ is an unsubstituted heterocycloalkyl. In embodiments, R⁴ is substituted or unsubstituted heterocycloalkyl (e.g., 3 to 8 membered, 3 to 6 membered, 4 to 6 membered, 4 to 5 membered, or 5 to 6 membered). In embodiments, R⁴ is substituted heterocycloalkyl (e.g., 3 to 8 membered, 3 to 6 membered, 4 to 6 membered, 4 to 5 membered, or 5 to 6 membered). In embodiments, R⁴ an unsubstituted heterocycloalkyl (e.g., 3 to 8 membered, 3 to 6 membered, 4 to 6 membered, 4 to 5 membered, or 5 to 6 membered).

In embodiments, R⁴ is substituted (e.g., substituted with a substituent group, a size-limited substituent group, or lower substituent group) or unsubstituted aryl. In embodiments, R⁴ is substituted (e.g., substituted with a substituent group, a size-limited substituent group, or lower substituent group) aryl. In embodiments, R⁴ is an unsubstituted aryl. In embodiments, R⁴ is substituted or unsubstituted aryl (e.g., C₆-C₁₀ or phenyl). In embodiments, R⁴ is substituted aryl (e.g., C₆-C₁₀ or phenyl). In embodiments, R⁴ is an unsubstituted aryl (e.g., C₆-C₁₀ or phenyl).

In embodiments, R⁴ is substituted (e.g., substituted with a substituent group, a size-limited substituent group, or lower substituent group) or unsubstituted heteroaryl. In embodiments, R⁴ is substituted (e.g., substituted with a substituent group, a size-limited substituent group, or lower substituent group) heteroaryl. In embodiments, R⁴ is an unsubstituted heteroaryl. In embodiments, R⁴ is substituted or unsubstituted heteroaryl (e.g., 5 to 10 membered, 5 to 9 membered, or 5 to 6 membered). In embodiments, R⁴ is substituted heteroaryl (e.g., 5 to 10 membered, 5 to 9 membered, or 5 to 6 membered). In embodiments, R⁴ is an unsubstituted heteroaryl (e.g., 5 to 10 membered, 5 to 9 membered, or 5 to 6 membered).

In embodiments, R⁴ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁴ is hydrogen, substituted or unsubstituted cycloalkyl, substituted or unsubstituted heterocycloalkyl, substituted or unsubstituted aryl, or substituted or unsubstituted heteroaryl.

In embodiments, R⁴ is hydrogen or R³⁴-substituted or unsubstituted alkyl (e.g., C₁-C₅ alkyl, C₁-C₆ alkyl, or C₁-C₄ alkyl), R³⁴-substituted or unsubstituted heteroalkyl (e.g., 2 to 8 membered heteroalkyl, 2 to 6 membered heteroalkyl, or 2 to 4 membered heteroalkyl), R³⁴-substituted or unsubstituted cycloalkyl (e.g., C₃-C₈ cycloalkyl, C₃-C₆ cycloalkyl, or C₅-C₆ cycloalkyl), R³⁴-substituted or unsubstituted heterocycloalkyl (e.g., 3 to 8 membered heterocycloalkyl, 3 to 6 membered heterocycloalkyl, or 5 to 6 membered heterocycloalkyl), R³⁴-substituted or unsubstituted aryl (e.g., C₆-C₁₀ aryl, C₁₀ aryl, or phenyl), or R³⁴-substituted or unsubstituted heteroaryl (e.g., 5 to 10 membered heteroaryl, 5 to 9 membered heteroaryl, or 5 to 6 membered heteroaryl).

In embodiments, R⁴ is R³⁴-substituted or unsubstituted alkyl (e.g., C₁-C₅ alkyl, C₁-C₆ alkyl, or C₁-C₄ alkyl). In embodiments, R⁴ is R³⁴-substituted alkyl (e.g., C₁-C₅ alkyl, C₁-C₆ alkyl, or C₁-C₄ alkyl). In embodiments, R⁴ is an unsubstituted alkyl (e.g., C₁-C₅ alkyl, C₁-C₆ alkyl, or C₁-C₄ alkyl).

In embodiments, R⁴ is R³⁴-substituted or unsubstituted heteroalkyl (e.g., 2 to 8 membered heteroalkyl, 2 to 6 membered heteroalkyl, or 2 to 4 membered heteroalkyl). In embodiments, R⁴ is R³⁴-substituted heteroalkyl (e.g., 2 to 8 membered heteroalkyl, 2 to 6 membered heteroalkyl, or 2 to 4 membered heteroalkyl). In embodiments, R⁴ is an unsubstituted heteroalkyl (e.g., 2 to 8 membered heteroalkyl, 2 to 6 membered heteroalkyl, or 2 to 4 membered heteroalkyl).

In embodiments, R⁴ is R³⁴-substituted or unsubstituted cycloalkyl (e.g., C₃-C₈ cycloalkyl, C₃-C₆ cycloalkyl, or C₅-C₆ cycloalkyl). In embodiments, R⁴ is R³⁴-substituted cycloalkyl (e.g., C₃-C₈ cycloalkyl, C₃-C₆ cycloalkyl, or C₅-C₆ cycloalkyl). In embodiments, R⁴ is an unsubstituted cycloalkyl (e.g., C₃-C₈ cycloalkyl, C₃-C₆ cycloalkyl, or C₅-C₆ cycloalkyl).

In embodiments, R⁴ is R³⁴-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁴ is R³⁴-substituted heterocycloalkyl (e.g., 3 to 8 membered heterocycloalkyl, 3 to 6 membered heterocycloalkyl, or 5 to 6 membered heterocycloalkyl). In embodiments, R⁴ is an unsubstituted heterocycloalkyl (e.g., 3 to 8 membered heterocycloalkyl, 3 to 6 membered heterocycloalkyl, or 5 to 6 membered heterocycloalkyl).

In embodiments, R⁴ is R³⁴-substituted or unsubstituted aryl (e.g., C₆-C₁₀ aryl, C₁₀ aryl, or phenyl). In embodiments, R⁴ is R³⁴-substituted aryl (e.g., C₆-C₁₀ aryl, C₁₀ aryl, or phenyl). In embodiments, R⁴ is an unsubstituted aryl (e.g., C₆-C₁₀ aryl, C₁₀ aryl, or phenyl).

In embodiments, R⁴ is R³⁴-substituted or unsubstituted heteroaryl (e.g., 5 to 10 membered heteroaryl, 5 to 9 membered heteroaryl, or 5 to 6 membered heteroaryl). In embodiments, R⁴ is R³⁴-substituted heteroaryl (e.g., 5 to 10 membered heteroaryl, 5 to 9 membered heteroaryl, or 5 to 6 membered heteroaryl). In embodiments, R⁴ is an unsubstituted heteroaryl (e.g., 5 to 10 membered heteroaryl, 5 to 9 membered heteroaryl, or 5 to 6 membered heteroaryl).

R³ is independently oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R⁶²-substituted or unsubstituted alkyl (e.g., C₁-C₅ alkyl, C₁-C₆ alkyl, or C₁-C₄ alkyl), R⁶²-substituted or unsubstituted heteroalkyl (e.g., 2 to 8 membered heteroalkyl, 2 to 6 membered heteroalkyl, or 2 to 4 membered heteroalkyl), R⁶²-substituted or unsubstituted cycloalkyl (e.g., C₃-C₈ cycloalkyl, C₃-C₆ cycloalkyl, or C₅-C₆ cycloalkyl), R⁶²-substituted or unsubstituted heterocycloalkyl (e.g., 3 to 8 membered heterocycloalkyl, 3 to 6 membered heterocycloalkyl, or 5 to 6 membered heterocycloalkyl), R⁶²-substituted or unsubstituted aryl (e.g., C₆-C₁₀ aryl, C₁₀ aryl, or phenyl), or R⁶²-substituted or unsubstituted heteroaryl (e.g., 5 to 10 membered heteroaryl, 5 to 9 membered heteroaryl, or 5 to 6 membered heteroaryl).

In embodiments, R⁴ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁴ is a substituted or unsubstituted C₁-C₆ alkyl. In embodiments, R⁴ is an unsubstituted C₁-C₃ alkyl. In embodiments, R⁴ is an unsubstituted methyl. In embodiments, R⁴ is

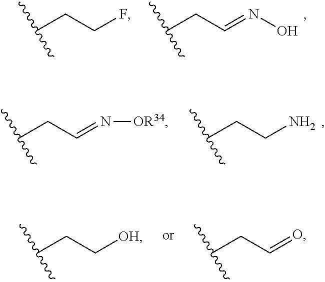

wherein R³⁴ is hydrogen, halogen, R⁶²-substituted or unsubstituted alkyl, R⁶²-substituted or unsubstituted heteroalkyl, R⁶²-substituted or unsubstituted cycloalkyl, R⁶²-substituted or unsubstituted heterocycloalkyl, R⁶²-substituted or unsubstituted aryl, or R⁶²-substituted or unsubstituted heteroaryl.

In embodiments, R³⁴ is substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R³⁴ is hydrogen.

In embodiments, R³⁴ is unsubstituted alkyl, unsubstituted heteroalkyl, unsubstituted cycloalkyl, unsubstituted heterocycloalkyl, unsubstituted aryl, or unsubstituted heteroaryl.

In embodiments, R³⁴ is R⁶²-substituted or unsubstituted alkyl (e.g., C₁-C₅ alkyl, C₁-C₆ alkyl, or C₁-C₄ alkyl), R⁶²-substituted or unsubstituted heteroalkyl (e.g., 2 to 8 membered heteroalkyl, 2 to 6 membered heteroalkyl, or 2 to 4 membered heteroalkyl), R⁶²-substituted or unsubstituted cycloalkyl (e.g., C₃-C₈ cycloalkyl, C₃-C₆ cycloalkyl, or C₅-C₆ cycloalkyl), R⁶²-substituted or unsubstituted heterocycloalkyl (e.g., 3 to 8 membered heterocycloalkyl, 3 to 6 membered heterocycloalkyl, or 5 to 6 membered heterocycloalkyl), R⁶²-substituted or unsubstituted aryl (e.g., C₆-C₁₀ aryl, C₁₀ aryl, or phenyl), or R⁶²-substituted or unsubstituted heteroaryl (e.g., 5 to 10 membered heteroaryl, 5 to 9 membered heteroaryl, or 5 to 6 membered heteroaryl).

In embodiments, R³⁴ is R⁶²-substituted or unsubstituted alkyl (e.g., C₁-C₅ alkyl, C₁-C₆ alkyl, or C₁-C₄ alkyl). In embodiments, R³⁴ is R⁶²-substituted alkyl (e.g., C₁-C₅ alkyl, C₁-C₆ alkyl, or C₁-C₄ alkyl). In embodiments, R³⁴ is an unsubstituted alkyl (e.g., C₁-C₈ alkyl, C₁-C₆ alkyl, or C₁-C₄ alkyl).

In embodiments, R³⁴ is R⁶²-substituted or unsubstituted heteroalkyl (e.g., 2 to 8 membered heteroalkyl, 2 to 6 membered heteroalkyl, or 2 to 4 membered heteroalkyl). In embodiments, R³ is R⁶²-substituted heteroalkyl (e.g., 2 to 8 membered heteroalkyl, 2 to 6 membered heteroalkyl, or 2 to 4 membered heteroalkyl). In embodiments, R³ is an unsubstituted heteroalkyl (e.g., 2 to 8 membered heteroalkyl, 2 to 6 membered heteroalkyl, or 2 to 4 membered heteroalkyl).

In embodiments, R³⁴ is R⁶²-substituted or unsubstituted cycloalkyl (e.g., C₃-C₈ cycloalkyl, C₃-C₆ cycloalkyl, or C₅-C₆ cycloalkyl). In embodiments, R³ is R⁶²-substituted cycloalkyl (e.g., C₃-C₈ cycloalkyl, C₃-C₆ cycloalkyl, or C₅-C₆ cycloalkyl). In embodiments, R³⁴ is an unsubstituted cycloalkyl (e.g., C₃-C₈ cycloalkyl, C₃-C₆ cycloalkyl, or C₅-C₆ cycloalkyl).

In embodiments, R³⁴ is R⁶²-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R³⁴ is R⁶²-substituted heterocycloalkyl (e.g., 3 to 8 membered heterocycloalkyl, 3 to 6 membered heterocycloalkyl, or 5 to 6 membered heterocycloalkyl). In embodiments, R³⁴ is an unsubstituted heterocycloalkyl (e.g., 3 to 8 membered heterocycloalkyl, 3 to 6 membered heterocycloalkyl, or 5 to 6 membered heterocycloalkyl).

In embodiments, R³⁴ is R⁶²-substituted or unsubstituted aryl (e.g., C₆-C₁₀ aryl, C₁₀ aryl, or phenyl). In embodiments, R³⁴ is R⁶²-substituted aryl (e.g., C₆-C₁₀ aryl, C₁₀ aryl, or phenyl). In embodiments, R³⁴ is an unsubstituted aryl (e.g., C₆-C₁₀ aryl, C₁₀ aryl, or phenyl).

In embodiments, R³⁴ is R⁶²-substituted or unsubstituted heteroaryl (e.g., 5 to 10 membered heteroaryl, 5 to 9 membered heteroaryl, or 5 to 6 membered heteroaryl). In embodiments, R³ is R⁶²-substituted heteroaryl (e.g., 5 to 10 membered heteroaryl, 5 to 9 membered heteroaryl, or 5 to 6 membered heteroaryl). In embodiments, R³⁴ is an unsubstituted heteroaryl (e.g., 5 to 10 membered heteroaryl, 5 to 9 membered heteroaryl, or 5 to 6 membered heteroaryl).

In embodiments, R⁵ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, or —N₃. In embodiments, R⁵ is hydrogen. In embodiments, R⁵ is oxo. In embodiments, R⁵ is halogen. In embodiments, R⁵ is —CCl₃. In embodiments, R⁵ is —CBr₃. In embodiments, R⁵ is —CF₃. In embodiments, R⁵ is —CI₃. In embodiments, R⁵ is CHCl₂. In embodiments, R⁵ is —CHBr₂. In embodiments, R⁵ is —CHF₂. In embodiments, R⁵ is —CHI₂. In embodiments, R⁵ is —CH₂C₁. In embodiments, R⁵ is —CH₂Br. In embodiments, R⁵ is —CH₂F. In embodiments, R⁵ is —CH₂I. In embodiments, R⁵ is —CN. In embodiments, R⁵ is —OH. In embodiments, R⁵ is —NH₂. In embodiments, R⁵ is —COOH. In embodiments, R⁵ is —CONH₂. In embodiments, R⁵ is —NO₂. In embodiments, R⁵ is —SH. In embodiments, R⁵ is —SO₃H. In embodiments, R⁵ is —SO₄H. In embodiments, R⁵ is —SO₂NH₂. In embodiments, R⁵ is —NHNH₂. In embodiments, R⁵ is —ONH₂. In embodiments, R⁵ is —NHC(O)NHNH₂. In embodiments, R⁵ is —NHC(O)NH₂. In embodiments, R⁵ is —NHSO₂H. In embodiments, R⁵ is —NHC(O)H. In embodiments, R⁵ is —NHC(O)OH. In embodiments, R⁵ is —NHOH. In embodiments, R⁵ is —OCCl₃. In embodiments, R⁵ is —OCF₃. In embodiments, R⁵ is —OCBr₃. In embodiments, R⁵ is —OCI₃. In embodiments, R⁵ is —OCHCl₂. In embodiments, R⁵ is —OCHBr₂. In embodiments, R⁵ is —OCHI₂. In embodiments, R⁵ is —OCHF₂. In embodiments, R⁵ is —OCH₂Cl. In embodiments, R⁵ is —OCH₂Br. In embodiments, R⁵ is —OCH₂I. In embodiments, R⁵ is —OCH₂F. In embodiments, R⁵ is —N₃. In embodiments, R⁵ is —OPO₃H. In embodiments, R⁵ is —OSO₃H.

In embodiments, R⁵ is a substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁵ is a R³³-substituted or unsubstituted alkyl, R³³-substituted or unsubstituted heteroalkyl, R³³-substituted or unsubstituted cycloalkyl, R³³-substituted or unsubstituted heterocycloalkyl, R³³-substituted or unsubstituted aryl, or R³³-substituted or unsubstituted heteroaryl.

In embodiments, R⁵ is R³³-substituted or unsubstituted alkyl (e.g., C₁-C₅ alkyl, C₁-C₆ alkyl, or C₁-C₄ alkyl), R³³-substituted or unsubstituted heteroalkyl (e.g., 2 to 8 membered heteroalkyl, 2 to 6 membered heteroalkyl, or 2 to 4 membered heteroalkyl), R³³-substituted or unsubstituted cycloalkyl (e.g., C₃-C₈ cycloalkyl, C₃-C₆ cycloalkyl, or C₅-C₆ cycloalkyl), R³³-substituted or unsubstituted heterocycloalkyl (e.g., 3 to 8 membered heterocycloalkyl, 3 to 6 membered heterocycloalkyl, or 5 to 6 membered heterocycloalkyl), R³³-substituted or unsubstituted aryl (e.g., C₆-C₁₀ aryl, C₁₀ aryl, or phenyl), or R³³-substituted or unsubstituted heteroaryl (e.g., 5 to 10 membered heteroaryl, 5 to 9 membered heteroaryl, or 5 to 6 membered heteroaryl).

In embodiments, R⁵ is R³³-substituted or unsubstituted alkyl (e.g., C₁-C₅ alkyl, C₁-C₆ alkyl, or C₁-C₄ alkyl). In embodiments, R⁵ is R³³-substituted alkyl (e.g., C₁-C₅ alkyl, C₁-C₆ alkyl, or C₁-C₄ alkyl). In embodiments, R⁵ is an unsubstituted alkyl (e.g., C₁-C₅ alkyl, C₁-C₆ alkyl, or C₁-C₄ alkyl).

In embodiments, R⁵ is R³³-substituted or unsubstituted heteroalkyl (e.g., 2 to 8 membered heteroalkyl, 2 to 6 membered heteroalkyl, or 2 to 4 membered heteroalkyl). In embodiments, R⁵ is R³³-substituted heteroalkyl (e.g., 2 to 8 membered heteroalkyl, 2 to 6 membered heteroalkyl, or 2 to 4 membered heteroalkyl). In embodiments, R⁵ is an unsubstituted heteroalkyl (e.g., 2 to 8 membered heteroalkyl, 2 to 6 membered heteroalkyl, or 2 to 4 membered heteroalkyl).

In embodiments, R⁵ is R³³-substituted or unsubstituted cycloalkyl (e.g., C₃-C₈ cycloalkyl, C₃-C₆ cycloalkyl, or C₅-C₆ cycloalkyl). In embodiments, R⁵ is R³³-substituted cycloalkyl (e.g., C₃-C₈ cycloalkyl, C₃-C₆ cycloalkyl, or C₅-C₆ cycloalkyl). In embodiments, R⁵ is an unsubstituted cycloalkyl (e.g., C₃-C₈ cycloalkyl, C₃-C₆ cycloalkyl, or C₅-C₆ cycloalkyl).

In embodiments, R⁵ is R³³-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁵ is R³³-substituted heterocycloalkyl (e.g., 3 to 8 membered heterocycloalkyl, 3 to 6 membered heterocycloalkyl, or 5 to 6 membered heterocycloalkyl). In embodiments, R⁵ is an unsubstituted heterocycloalkyl (e.g., 3 to 8 membered heterocycloalkyl, 3 to 6 membered heterocycloalkyl, or 5 to 6 membered heterocycloalkyl).

In embodiments, R⁵ is R³³-substituted or unsubstituted aryl (e.g., C₆-C₁₀ aryl, C₁₀ aryl, or phenyl). In embodiments, R⁵ is R³³-substituted aryl (e.g., C₆-C₁₀ aryl, C₁₀ aryl, or phenyl). In embodiments, R⁵ is an unsubstituted aryl (e.g., C₆-C₁₀ aryl, C₁₀ aryl, or phenyl).

In embodiments, R⁵ is R³³-substituted or unsubstituted heteroaryl (e.g., 5 to 10 membered heteroaryl, 5 to 9 membered heteroaryl, or 5 to 6 membered heteroaryl). In embodiments, R⁵ is R³³-substituted heteroaryl (e.g., 5 to 10 membered heteroaryl, 5 to 9 membered heteroaryl, or 5 to 6 membered heteroaryl). In embodiments, R⁵ is an unsubstituted heteroaryl (e.g., 5 to 10 membered heteroaryl, 5 to 9 membered heteroaryl, or 5 to 6 membered heteroaryl).

In embodiments, R⁵ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁵ is hydrogen, substituted or unsubstituted cycloalkyl, substituted or unsubstituted heterocycloalkyl, substituted or unsubstituted aryl, or substituted or unsubstituted heteroaryl.

In embodiments, R⁵ is a substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁵ is an unsubstituted C₁-C₆ alkyl. In embodiments, R⁵ is an unsubstituted C₃ alkyl. In embodiments, R⁵ is an unsubstituted isopropyl.

In embodiments, R⁵ is:

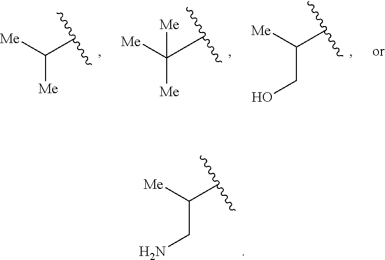

In embodiments, R⁵ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃, —SO₄, —SO₂NH₂, —NHNH₂, —ONH₂, —NHC(O)NHNH₂, —NHC(O)NH₂, —NHSO₂H, —NHC(O), —NHC(O)OH, —NHOH, —OCCl₃, —OCF₃, —OCBr₃, —OCI₃, —OCHC₂, —OCHBr₂, —OCHI₂, —OCHF₂, —OCH₂Cl, —OCH₂Br, —OCH₂I, —OCH₂F, —N₃, or substituted or unsubstituted alkyl, or substituted or unsubstituted heteroalkyl. In embodiments, R⁵ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁵ is substituted or unsubstituted C₁-C₅ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, R⁵ is substituted or unsubstituted C₁-C₈ alkyl. In embodiments, R⁵ is substituted or unsubstituted C₁-C₃ alkyl.

R³³ is oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R³³ is a substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R³³ is substituted (e.g., substituted with a substituent group, a size-limited substituent group, or lower substituent group) or unsubstituted alkyl. In embodiments, R³³ is substituted (e.g., substituted with a substituent group, a size-limited substituent group, or lower substituent group) alkyl. In embodiments, R³³ is unsubstituted alkyl. In embodiments, R³³ is substituted or unsubstituted alkyl (e.g., C₁-C₅, C₁-C₆, C₁-C₄, or C₁-C₂). In embodiments, R³³ is substituted alkyl (e.g., C₁-C₅, C₁-C₆, C₁-C₄, or C₁-C₂). In embodiments, R³³ is unsubstituted alkyl (e.g., C₁-C₅, C₁-C₆, C₁-C₄, or C₁-C₂).

In embodiments, R³³ is substituted (e.g., substituted with a substituent group, a size-limited substituent group, or lower substituent group) or unsubstituted heteroalkyl. In embodiments, R³³ is substituted (e.g., substituted with a substituent group, a size-limited substituent group, or lower substituent group) heteroalkyl. In embodiments, R³³ is unsubstituted heteroalkyl. In embodiments, R³³ is substituted or unsubstituted heteroalkyl (e.g., 2 to 8 membered, 2 to 6 membered, 4 to 6 membered, 2 to 3 membered, or 4 to 5 membered). In embodiments, R³³ is substituted heteroalkyl (e.g., 2 to 8 membered, 2 to 6 membered, 4 to 6 membered, 2 to 3 membered, or 4 to 5 membered). In embodiments, R³³ is an unsubstituted heteroalkyl (e.g., 2 to 8 membered, 2 to 6 membered, 4 to 6 membered, 2 to 3 membered, or 4 to 5 membered).

In embodiments, R³³ is substituted (e.g., substituted with a substituent group, a size-limited substituent group, or lower substituent group) or unsubstituted cycloalkyl. In embodiments, R³³ is substituted (e.g., substituted with a substituent group, a size-limited substituent group, or lower substituent group) cycloalkyl. In embodiments, R³³ is an unsubstituted cycloalkyl. In embodiments, R³³ is substituted or unsubstituted cycloalkyl (e.g., C₃-C₈, C₃-C₆, C₄-C₆, or C₅-C₆). In embodiments, R³³ is substituted cycloalkyl (e.g., C₃-C₅, C₃-C₆, C₄-C₆, or C₅-C₆). In embodiments, R³³ is unsubstituted cycloalkyl (e.g., C₃-C₈, C₃-C₆, C₄-C₆, or C₅-C₆).

In embodiments, R³³ is substituted (e.g., substituted with a substituent group, a size-limited substituent group, or lower substituent group) or unsubstituted heterocycloalkyl. In embodiments, R³³ is substituted (e.g., substituted with a substituent group, a size-limited substituent group, or lower substituent group) heterocycloalkyl. In embodiments, R³³ is an unsubstituted heterocycloalkyl. In embodiments, R³³ is substituted or unsubstituted heterocycloalkyl (e.g., 3 to 8 membered, 3 to 6 membered, 4 to 6 membered, 4 to 5 membered, or 5 to 6 membered). In embodiments, R³³ is substituted heterocycloalkyl (e.g., 3 to 8 membered, 3 to 6 membered, 4 to 6 membered, 4 to 5 membered, or 5 to 6 membered). In embodiments, R³³ an unsubstituted heterocycloalkyl (e.g., 3 to 8 membered, 3 to 6 membered, 4 to 6 membered, 4 to 5 membered, or 5 to 6 membered).

In embodiments, R³³ is substituted (e.g., substituted with a substituent group, a size-limited substituent group, or lower substituent group) or unsubstituted aryl. In embodiments, R³³ is substituted (e.g., substituted with a substituent group, a size-limited substituent group, or lower substituent group) aryl. In embodiments, R³³ is an unsubstituted aryl. In embodiments, R³³ is substituted or unsubstituted aryl (e.g., C₆-C₁₀ or phenyl). In embodiments, R³³ is substituted aryl (e.g., C₆-C₁₀ or phenyl). In embodiments, R³³ is an unsubstituted aryl (e.g., C₆-C₁₀ or phenyl).

In embodiments, R³³ is substituted (e.g., substituted with a substituent group, a size-limited substituent group, or lower substituent group) or unsubstituted heteroaryl. In embodiments, R³³ is substituted (e.g., substituted with a substituent group, a size-limited substituent group, or lower substituent group) heteroaryl. In embodiments, R³³ is an unsubstituted heteroaryl. In embodiments, R³³ is substituted or unsubstituted heteroaryl (e.g., 5 to 10 membered, 5 to 9 membered, or 5 to 6 membered). In embodiments, R³³ is substituted heteroaryl (e.g., 5 to 10 membered, 5 to 9 membered, or 5 to 6 membered). In embodiments, R³³ is an unsubstituted heteroaryl (e.g., 5 to 10 membered, 5 to 9 membered, or 5 to 6 membered).

In embodiments, R³³ is oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R³-substituted or unsubstituted alkyl (e.g., C₁-C₅ alkyl, C₁-C₆ alkyl, or C₁-C₄ alkyl), R³-substituted or unsubstituted heteroalkyl (e.g., 2 to 8 membered heteroalkyl, 2 to 6 membered heteroalkyl, or 2 to 4 membered heteroalkyl), R³⁶-substituted or unsubstituted cycloalkyl (e.g., C₃-C₈ cycloalkyl, C₃-C₆ cycloalkyl, or C₅-C₆ cycloalkyl), R³⁶-substituted or unsubstituted heterocycloalkyl (e.g., 3 to 8 membered heterocycloalkyl, 3 to 6 membered heterocycloalkyl, or 5 to 6 membered heterocycloalkyl), R⁵⁰-substituted or unsubstituted aryl (e.g., C₆-C₁₀ aryl, C₁₀ aryl, or phenyl), or R³⁶-substituted or unsubstituted heteroaryl (e.g., 5 to 10 membered heteroaryl, 5 to 9 membered heteroaryl, or 5 to 6 membered heteroaryl).

In embodiments, R³³ is R³⁶-substituted or unsubstituted alkyl (e.g., C₁-C₅ alkyl, C₁-C₆ alkyl, or C₁-C₄ alkyl), R⁵⁰-substituted or unsubstituted heteroalkyl (e.g., 2 to 8 membered heteroalkyl, 2 to 6 membered heteroalkyl, or 2 to 4 membered heteroalkyl), R³⁶-substituted or unsubstituted cycloalkyl (e.g., C₃-C₈ cycloalkyl, C₃-C₆ cycloalkyl, or C₅-C₆ cycloalkyl), R³⁶-substituted or unsubstituted heterocycloalkyl (e.g., 3 to 8 membered heterocycloalkyl, 3 to 6 membered heterocycloalkyl, or 5 to 6 membered heterocycloalkyl), R³⁶-substituted or unsubstituted aryl (e.g., C₆-C₁₀ aryl, C₁₀ aryl, or phenyl), or R³⁶-substituted or unsubstituted heteroaryl (e.g., 5 to 10 membered heteroaryl, 5 to 9 membered heteroaryl, or 5 to 6 membered heteroaryl).

In embodiments, R³³ is R³⁶-substituted or unsubstituted alkyl (e.g., C₁-C₅ alkyl, C₁-C₆ alkyl, or C₁-C₄ alkyl). In embodiments, R³³ is R³⁶-substituted alkyl (e.g., C₁-C₅ alkyl, C₁-C₆ alkyl, or C₁-C₄ alkyl). In embodiments, R³³ is an unsubstituted alkyl (e.g., C₁-C₅ alkyl, C₁-C₆ alkyl, or C₁-C₄ alkyl).

In embodiments, R³³ is R³⁶-substituted or unsubstituted heteroalkyl (e.g., 2 to 8 membered heteroalkyl, 2 to 6 membered heteroalkyl, or 2 to 4 membered heteroalkyl). In embodiments, R³³ is R³⁶-substituted heteroalkyl (e.g., 2 to 8 membered heteroalkyl, 2 to 6 membered heteroalkyl, or 2 to 4 membered heteroalkyl). In embodiments, R³³ is an unsubstituted heteroalkyl (e.g., 2 to 8 membered heteroalkyl, 2 to 6 membered heteroalkyl, or 2 to 4 membered heteroalkyl).

In embodiments, R³³ is R³⁶-substituted or unsubstituted cycloalkyl (e.g., C₃-C₈ cycloalkyl, C₃-C₆ cycloalkyl, or C₅-C₆ cycloalkyl). In embodiments, R³³ is R⁵⁰-substituted cycloalkyl (e.g., C₃-C₈ cycloalkyl, C₃-C₆ cycloalkyl, or C₅-C₆ cycloalkyl). In embodiments, R³³ is an unsubstituted cycloalkyl (e.g., C₃-C₈ cycloalkyl, C₃-C₆ cycloalkyl, or C₅-C₆ cycloalkyl).

In embodiments, R³³ is R³⁶-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R³³ is R³⁶-substituted heterocycloalkyl (e.g., 3 to 8 membered heterocycloalkyl, 3 to 6 membered heterocycloalkyl, or 5 to 6 membered heterocycloalkyl). In embodiments, R³³ is an unsubstituted heterocycloalkyl (e.g., 3 to 8 membered heterocycloalkyl, 3 to 6 membered heterocycloalkyl, or 5 to 6 membered heterocycloalkyl).

In embodiments, R³³ is R³⁶-substituted or unsubstituted aryl (e.g., C₆-C₁₀ aryl, C₁₀ aryl, or phenyl). In embodiments, R³³ is R³⁶-substituted aryl (e.g., C₆-C₁₀ aryl, C₁₀ aryl, or phenyl). In embodiments, R³³ is an unsubstituted aryl (e.g., C₆-C₁₀ aryl, C₁₀ aryl, or phenyl).

In embodiments, R³³ is R³⁶-substituted or unsubstituted heteroaryl (e.g., 5 to 10 membered heteroaryl, 5 to 9 membered heteroaryl, or 5 to 6 membered heteroaryl). In embodiments, R³³ is R⁵⁰-substituted heteroaryl (e.g., 5 to 10 membered heteroaryl, 5 to 9 membered heteroaryl, or 5 to 6 membered heteroaryl). In embodiments, R³³ is an unsubstituted heteroaryl (e.g., 5 to 10 membered heteroaryl, 5 to 9 membered heteroaryl, or 5 to 6 membered heteroaryl).

R³ is independently oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R³⁷-substituted or unsubstituted alkyl (e.g., C₁-C₅ alkyl, C₁-C₆ alkyl, or C₁-C₄ alkyl), R³⁷-substituted or unsubstituted heteroalkyl (e.g., 2 to 8 membered heteroalkyl, 2 to 6 membered heteroalkyl, or 2 to 4 membered heteroalkyl), R³⁷-substituted or unsubstituted cycloalkyl (e.g., C₃-C₈ cycloalkyl, C₃-C₆ cycloalkyl, or C₅-C₆ cycloalkyl), R³⁷-substituted or unsubstituted heterocycloalkyl (e.g., 3 to 8 membered heterocycloalkyl, 3 to 6 membered heterocycloalkyl, or 5 to 6 membered heterocycloalkyl), R³⁷-substituted or unsubstituted aryl (e.g., C₆-C₁₀ aryl, C₁₀ aryl, or phenyl), or R³⁷-substituted or unsubstituted heteroaryl (e.g., 5 to 10 membered heteroaryl, 5 to 9 membered heteroaryl, or 5 to 6 membered heteroaryl).

In embodiments, R³⁶ is R³⁷-substituted or unsubstituted alkyl (e.g., C₁-C₅ alkyl, C₁-C₆ alkyl, or C₁-C₄ alkyl), R³⁷-substituted or unsubstituted heteroalkyl (e.g., 2 to 8 membered heteroalkyl, 2 to 6 membered heteroalkyl, or 2 to 4 membered heteroalkyl), R³⁷-substituted or unsubstituted cycloalkyl (e.g., C₃-C₈ cycloalkyl, C₃-C₆ cycloalkyl, or C₅-C₆ cycloalkyl), R³⁷-substituted or unsubstituted heterocycloalkyl (e.g., 3 to 8 membered heterocycloalkyl, 3 to 6 membered heterocycloalkyl, or 5 to 6 membered heterocycloalkyl), R³⁷-substituted or unsubstituted aryl (e.g., C₆-C₁₀ aryl, C₁₀ aryl, or phenyl), or R³⁷-substituted or unsubstituted heteroaryl (e.g., 5 to 10 membered heteroaryl, 5 to 9 membered heteroaryl, or 5 to 6 membered heteroaryl).

In embodiments, R³⁶ is R³⁷-substituted or unsubstituted alkyl (e.g., C₁-C₅ alkyl, C₁-C₆ alkyl, or C₁-C₄ alkyl). In embodiments, R³⁶ is R³⁷-substituted alkyl (e.g., C₁-C₅ alkyl, C₁-C₆ alkyl, or C₁-C₄ alkyl). In embodiments, R³⁶ is an unsubstituted alkyl (e.g., C₁-C₅ alkyl, C₁-C₆ alkyl, or C₁-C₄ alkyl).

In embodiments, R³⁶ is R³⁷-substituted or unsubstituted heteroalkyl (e.g., 2 to 8 membered heteroalkyl, 2 to 6 membered heteroalkyl, or 2 to 4 membered heteroalkyl). In embodiments, R¹ is R³⁷-substituted heteroalkyl (e.g., 2 to 8 membered heteroalkyl, 2 to 6 membered heteroalkyl, or 2 to 4 membered heteroalkyl). In embodiments, R³ is an unsubstituted heteroalkyl (e.g., 2 to 8 membered heteroalkyl, 2 to 6 membered heteroalkyl, or 2 to 4 membered heteroalkyl).

In embodiments, R³⁶ is R³⁷-substituted or unsubstituted cycloalkyl (e.g., C₃-C₈ cycloalkyl, C₃-C₆ cycloalkyl, or C₅-C₆ cycloalkyl). In embodiments, R³ is R³⁷-substituted cycloalkyl (e.g., C₃-C₈ cycloalkyl, C₃-C₆ cycloalkyl, or C₅-C₆ cycloalkyl). In embodiments, R³⁶ is an unsubstituted cycloalkyl (e.g., C₃-C₈ cycloalkyl, C₃-C₆ cycloalkyl, or C₅-C₆ cycloalkyl).

In embodiments, R³⁶ is R³⁷-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R³⁶ is R³⁷-substituted heterocycloalkyl (e.g., 3 to 8 membered heterocycloalkyl, 3 to 6 membered heterocycloalkyl, or 5 to 6 membered heterocycloalkyl). In embodiments, R³⁶ is an unsubstituted heterocycloalkyl (e.g., 3 to 8 membered heterocycloalkyl, 3 to 6 membered heterocycloalkyl, or 5 to 6 membered heterocycloalkyl).

In embodiments, R³⁶ is R³⁷-substituted or unsubstituted aryl (e.g., C₆-C₁₀ aryl, C₁₀ aryl, or phenyl). In embodiments, R³⁶ is R³⁷-substituted aryl (e.g., C₆-C₁₀ aryl, C₁₀ aryl, or phenyl). In embodiments, R³⁶ is an unsubstituted aryl (e.g., C₆-C₁₀ aryl, C₁₀ aryl, or phenyl).

In embodiments, R³⁶ is R³⁷-substituted or unsubstituted heteroaryl (e.g., 5 to 10 membered heteroaryl, 5 to 9 membered heteroaryl, or 5 to 6 membered heteroaryl). In embodiments, R³⁶ is R³⁷-substituted heteroaryl (e.g., 5 to 10 membered heteroaryl, 5 to 9 membered heteroaryl, or 5 to 6 membered heteroaryl). In embodiments, R³⁶ is an unsubstituted heteroaryl (e.g., 5 to 10 membered heteroaryl, 5 to 9 membered heteroaryl, or 5 to 6 membered heteroaryl).

In embodiments, R⁵ is an unsubstituted C₄ alkyl. In embodiments, R⁵ is an unsubstituted C₃ alkyl. In embodiments, R⁵ is isopropyl. In embodiments, R⁵ is t-butyl. In embodiments, R⁵ is

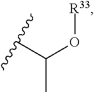

wherein R³³ is as described herein. In embodiments R⁵ is

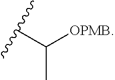

In embodiments, R⁵ is

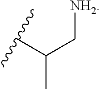 [SMILES: *C(C)CN]

In embodiments, R⁵ is

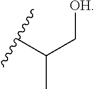 [SMILES: *C(C)CO]

In embodiments, R⁵ is

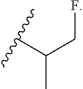 [SMILES: *C(C)CF]

In embodiments, R⁵ is

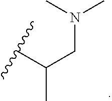 [SMILES: *C(C)CN(C)C]

In embodiments, R⁵ is

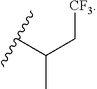 [SMILES: *C(C)CC(F)(F)F]

In embodiments, R⁵ is

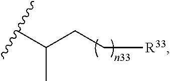

wherein n33 is an integer from 0 to 20. In embodiments, n33 is an integer from 0 to 4.

In embodiments, R⁵ is

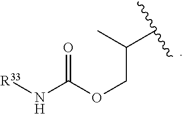

In embodiments, R⁵ is

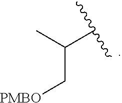

In embodiments, R⁵ is

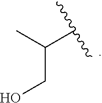 [SMILES: *C(C)CO]

embodiments, R⁵ is

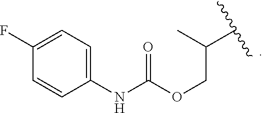 [SMILES: *C(C)COC(=O)Nc1ccc(F)cc1]

In embodiments, R⁵ is

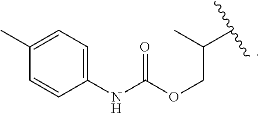 [SMILES: *C(C)COC(=O)Nc1ccc(C)cc1]

In embodiments, R⁵ is

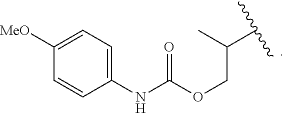 [SMILES: *C(C)COC(=O)Nc1ccc(OC)cc1]

In embodiments, R⁵ is

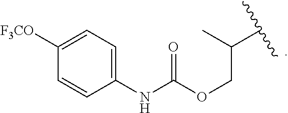 [SMILES: *C(C)COC(=O)Nc1ccc(OC(F)(F)F)cc1]

In embodiments, R⁵ is

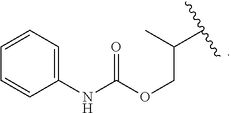 [SMILES: *C(C)COC(=O)Nc1ccccc1]

In embodiments, R⁵ is

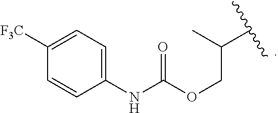 [SMILES: *C(C)COC(=O)Nc1ccc(C(F)(F)F)cc1]

In embodiments, R⁵ is

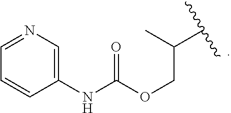 [SMILES: *C(C)COC(=O)Nc1cccnc1]

In embodiments, R⁵ is

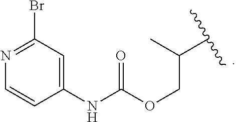 [SMILES: *C(C)COC(=O)Nc1ccnc(Br)c1]

In embodiments, R⁵ is

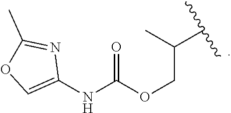 [SMILES: *C(C)COC(=O)Nc1coc(C)n1]

In embodiments, R⁵ is

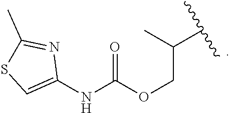 [SMILES: *C(C)COC(=O)Nc1csc(C)n1]

In embodiments, R⁵ is

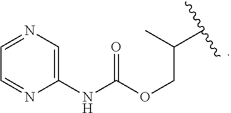 [SMILES: *C(C)COC(=O)Nc1cnccn1]

In embodiments, R⁵ is

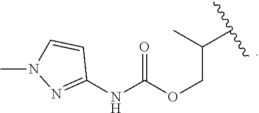 [SMILES: *C(C)COC(=O)Nc1ccn(C)n1]

In embodiments, R⁵ is

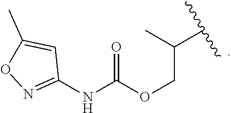 [SMILES: *C(C)COC(=O)Nc1cc(C)on1]

In embodiments, R⁵ is

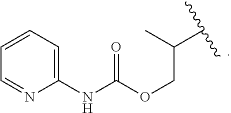 [SMILES: *C(C)COC(=O)Nc1ccccn1]

In embodiments, R⁵ is

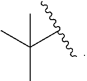 [SMILES: *C(C)(C)C]

In embodiments, R⁵ is

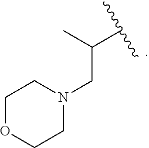 [SMILES: *C(C)CN1CCOCC1]

In embodiments, R⁵ is

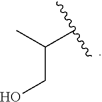 [SMILES: *C(C)CO]

In embodiments, R⁵ is

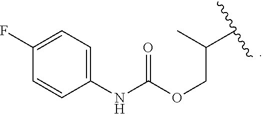 [SMILES: *C(C)COC(=O)Nc1ccc(F)cc1]

In embodiments, R⁵ is

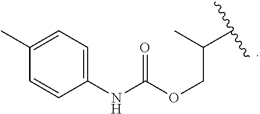 [SMILES: *C(C)COC(=O)Nc1ccc(C)cc1]

In embodiments, R⁵ is

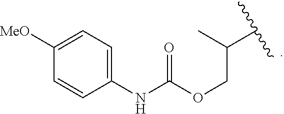 [SMILES: *C(C)COC(=O)Nc1ccc(OC)cc1]

In embodiments, R⁵ is

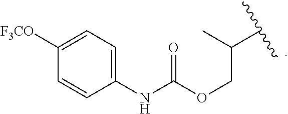 [SMILES: *C(C)COC(=O)Nc1ccc(OC(F)(F)F)cc1]

In embodiments, R⁵ is

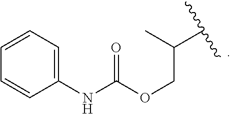 [SMILES: *C(C)COC(=O)Nc1ccccc1]

In embodiments, R⁵ is

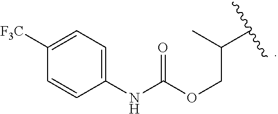 [SMILES: *C(C)COC(=O)Nc1ccc(C(F)(F)F)cc1]

In embodiments, R⁵ is

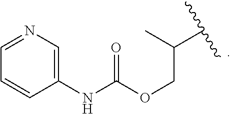 [SMILES: *C(C)COC(=O)Nc1cccnc1]

In embodiments, R⁵ is

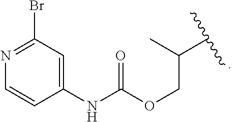 [SMILES: *C(C)COC(=O)Nc1ccnc(Br)c1]

In embodiments, R⁵ is

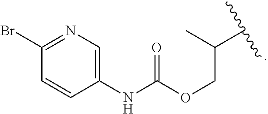 [SMILES: *C(C)COC(=O)Nc1ccc(Br)nc1]

In embodiments, R⁵ is

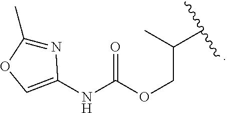 [SMILES: *C(C)COC(=O)Nc1coc(C)n1]

In embodiments, R⁵ is

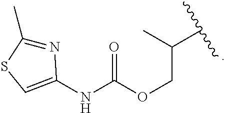 [SMILES: *C(C)COC(=O)Nc1csc(C)n1]

In embodiments, R⁵ is

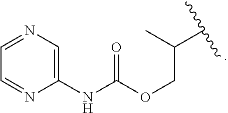 [SMILES: *C(C)COC(=O)Nc1cnccn1]

In embodiments, R⁵ is

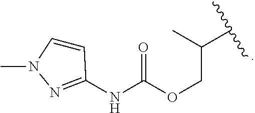 [SMILES: *C(C)COC(=O)Nc1ccn(C)n1]

In embodiments, R⁵ is

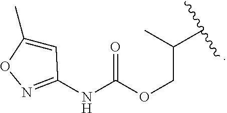 [SMILES: *C(C)COC(=O)Nc1cc(C)on1]

In embodiments, R⁵ is

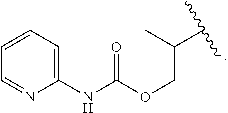 [SMILES: *C(C)COC(=O)Nc1ccccn1]

In embodiments, R⁵ is

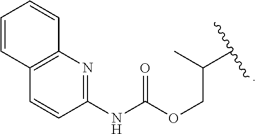 [SMILES: *C(C)COC(=O)Nc1ccc2ccccc2n1]

In embodiments, R⁵ is

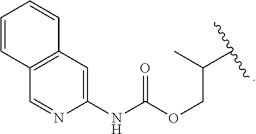 [SMILES: *C(C)COC(=O)Nc1cc2ccccc2cn1]

In embodiments, R⁵ is

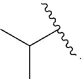 [SMILES: *C(C)C]

In embodiments, R⁵ is

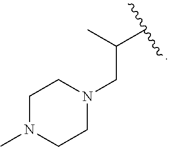 [SMILES: *C(C)CN1CCN(C)CC1]

In embodiments, R⁵ is

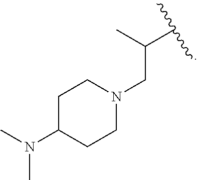 [SMILES: *C(C)CN1CCC(N(C)C)CC1]

In embodiments, R⁵ is

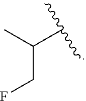 [SMILES: *C(C)CF]

In embodiments, R⁵ is:

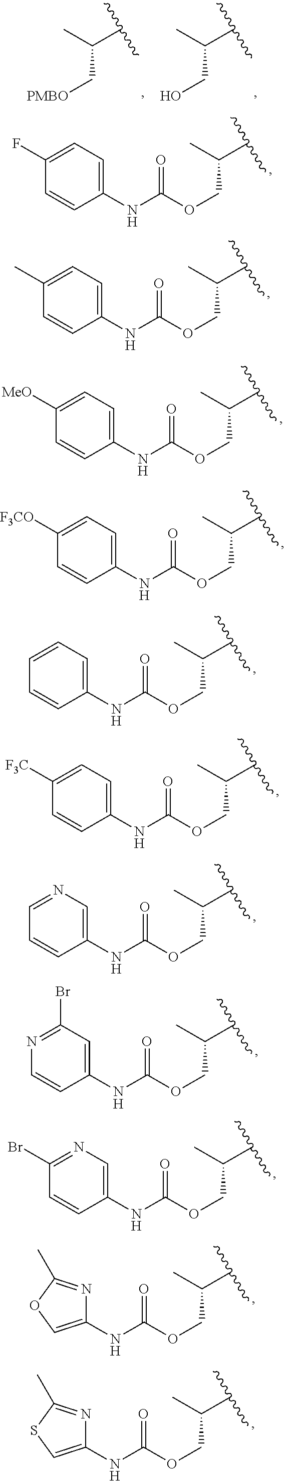

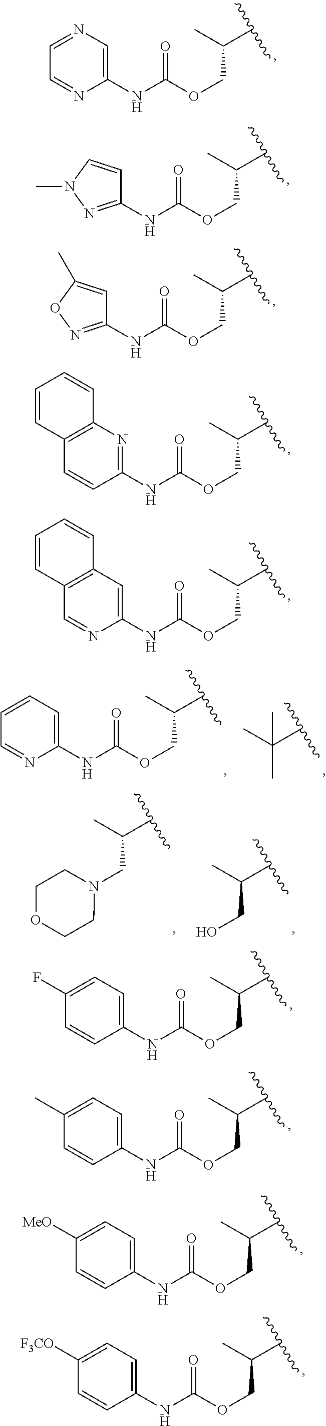

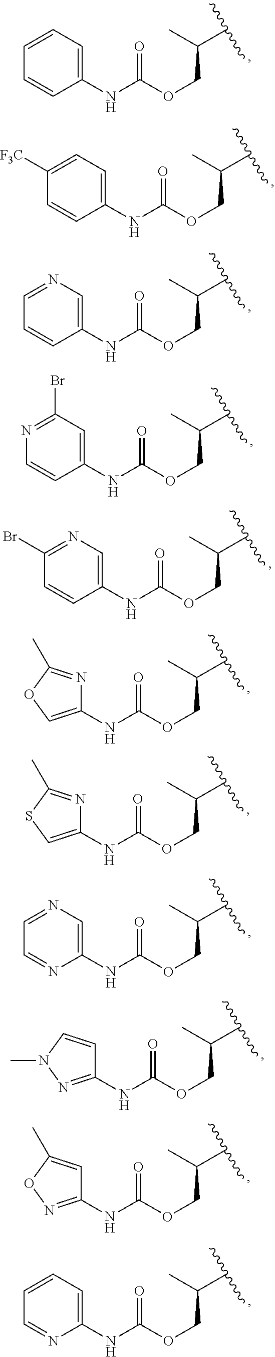

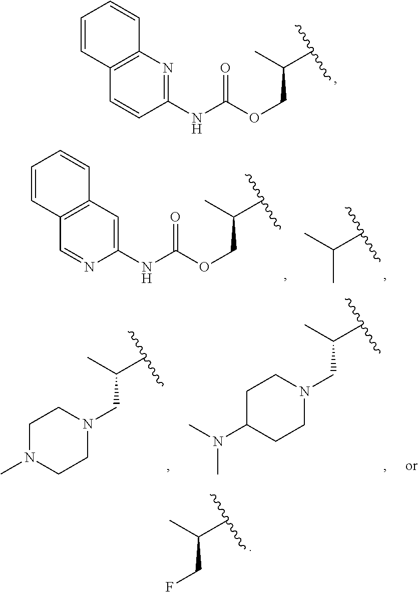

In embodiments, R⁵ is

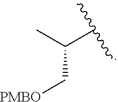

In embodiments, R⁵ is

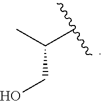 [SMILES: *[C@@H](C)CO]

In embodiments, R⁵ is

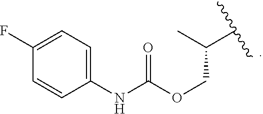 [SMILES: *[C@@H](C)COC(=O)Nc1ccc(F)cc1]

In embodiments, R⁵ is

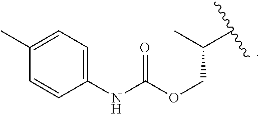 [SMILES: *[C@@H](C)COC(=O)Nc1ccc(C)cc1]

In embodiments, R⁵ is

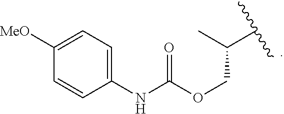 [SMILES: *[C@@H](C)COC(=O)Nc1ccc(OC)cc1]

In embodiments, R⁵ is

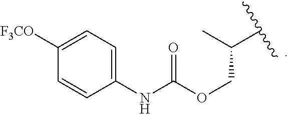 [SMILES: *[C@@H](C)COC(=O)Nc1ccc(OC(F)(F)F)cc1]

In embodiments, R⁵ is

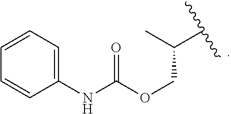 [SMILES: *[C@@H](C)COC(=O)Nc1ccccc1]

In embodiments, R⁵ is

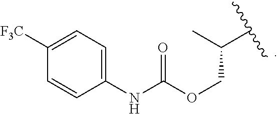 [SMILES: *[C@@H](C)COC(=O)Nc1ccc(C(F)(F)F)cc1]

In embodiments, R⁵ is

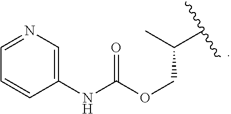 [SMILES: *[C@@H](C)COC(=O)Nc1cccnc1]

In embodiments, R⁵ is

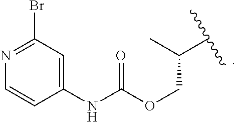 [SMILES: *[C@@H](C)COC(=O)Nc1ccnc(Br)c1]

In embodiments, R⁵ is

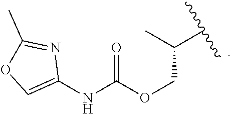 [SMILES: *[C@@H](C)COC(=O)Nc1coc(C)n1]

In embodiments, R⁵ is

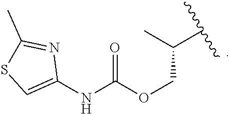 [SMILES: *[C@@H](C)COC(=O)Nc1csc(C)n1]

In embodiments, R⁵ is

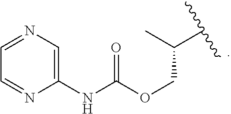 [SMILES: *[C@@H](C)COC(=O)Nc1cnccn1]

In embodiments, R⁵ is

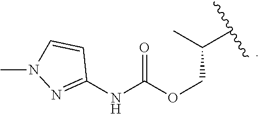 [SMILES: *[C@@H](C)COC(=O)Nc1ccn(C)n1]

In embodiments, R⁵ is

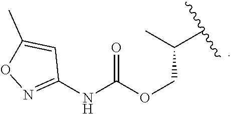 [SMILES: *[C@@H](C)COC(=O)Nc1cc(C)on1]

In embodiments, R⁵ is

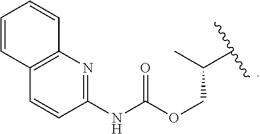 [SMILES: *[C@@H](C)COC(=O)Nc1ccc2ccccc2n1]

In embodiments, R⁵ is

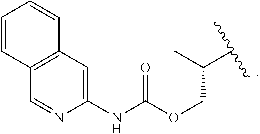 [SMILES: *[C@@H](C)COC(=O)Nc1cc2ccccc2cn1]

In embodiments, R⁵ is

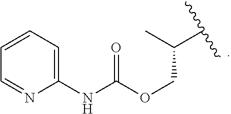 [SMILES: *[C@@H](C)COC(=O)Nc1ccccn1]

In embodiments, R⁵ is

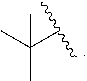 [SMILES: *C(C)(C)C]

In embodiments, R⁵ is

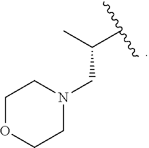 [SMILES: *[C@@H](C)CN1CCOCC1]

In embodiments, R⁵ is

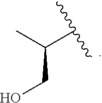 [SMILES: *[C@H](C)CO]

In embodiments, R⁵ is

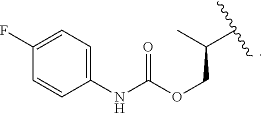 [SMILES: *[C@H](C)COC(=O)Nc1ccc(F)cc1]

In embodiments, R⁵ is

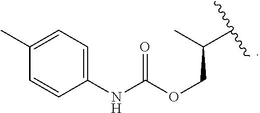 [SMILES: *[C@H](C)COC(=O)Nc1ccc(C)cc1]

In embodiments, R⁵ is

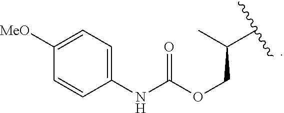 [SMILES: *[C@H](C)COC(=O)Nc1ccc(OC)cc1]

In embodiments, R⁵ is

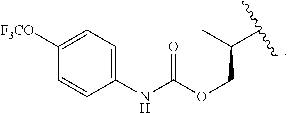 [SMILES: *[C@H](C)COC(=O)Nc1ccc(OC(F)(F)F)cc1]

In embodiments, R⁵ is

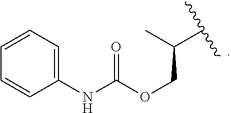 [SMILES: *[C@H](C)COC(=O)Nc1ccccc1]

In embodiments, R⁵ is

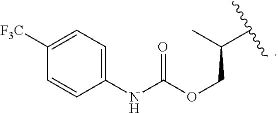 [SMILES: *[C@H](C)COC(=O)Nc1ccc(C(F)(F)F)cc1]

In embodiments, R⁵ is

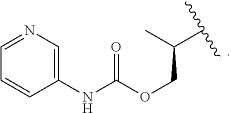 [SMILES: *[C@H](C)COC(=O)Nc1cccnc1]

In embodiments, R⁵ is

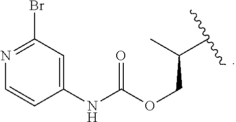 [SMILES: *[C@H](C)COC(=O)Nc1ccnc(Br)c1]

In embodiments, R⁵ is

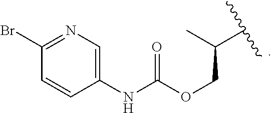 [SMILES: *[C@H](C)COC(=O)Nc1ccc(Br)nc1]

In embodiments, R⁵ is

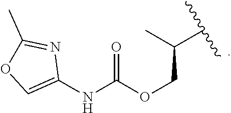 [SMILES: *[C@H](C)COC(=O)Nc1coc(C)n1]

In embodiments, R⁵ is

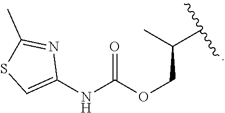 [SMILES: *[C@H](C)COC(=O)Nc1csc(C)n1]

In embodiments, R⁵ is

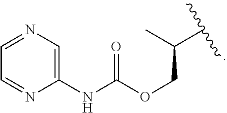 [SMILES: *[C@H](C)COC(=O)Nc1cnccn1]

In embodiments, R⁵ is

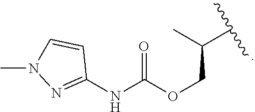 [SMILES: *[C@H](C)COC(=O)Nc1ccn(C)n1]

In embodiments, R⁵ is

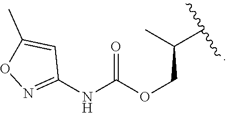 [SMILES: *[C@H](C)COC(=O)Nc1cc(C)on1]

In embodiments, R⁵ is

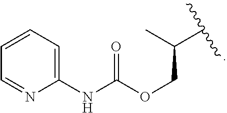 [SMILES: *[C@H](C)COC(=O)Nc1ccccn1]

In embodiments, R⁵ is

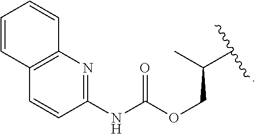 [SMILES: *[C@H](C)COC(=O)Nc1ccc2ccccc2n1]

In embodiments, R⁵ is

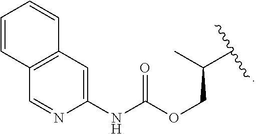 [SMILES: *[C@H](C)COC(=O)Nc1cc2ccccc2cn1]

In embodiments, R⁵ is

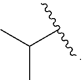 [SMILES: *C(C)C]

In embodiments, R⁵ is

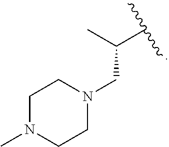 [SMILES: *[C@@H](C)CN1CCN(C)CC1]

In embodiments, R⁵ is

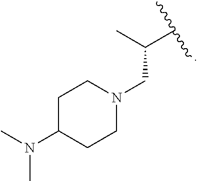 [SMILES: *[C@@H](C)CN1CCC(N(C)C)CC1]

In embodiments, R⁵ is

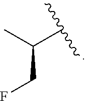 [SMILES: *[C@H](C)CF]

In embodiments, R⁶ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, or —N₃. In embodiments, R⁶ is hydrogen. In embodiments, R⁶ is oxo. In embodiments, R⁶ is halogen. In embodiments, R⁶ is —CCl₃. In embodiments, R⁶ is —CBr₃. In embodiments, R⁶ is —CF₃. In embodiments, R⁶ is —CI₃. In embodiments, R⁶ is CHCl₂. In embodiments, R⁶ is —CHBr₂. In embodiments, R⁶ is —CHF₂. In embodiments, R⁶ is —CHI₂. In embodiments, R⁶ is —CH₂C₁. In embodiments, R⁶ is —CH₂Br. In embodiments, R⁶ is —CH₂F. In embodiments, R⁶ is —CH₂I. In embodiments, R⁶ is —CN. In embodiments, R⁶ is —OH. In embodiments, R⁶ is —NH₂. In embodiments, R⁶ is —COOH. In embodiments, R⁶ is —CONH₂. In embodiments, R⁶ is —NO₂. In embodiments, R⁶ is —SH. In embodiments, R⁶ is —SO₃H. In embodiments, R⁶ is —SO₄H. In embodiments, R⁶ is —SO₂NH₂. In embodiments, R⁶ is —NHNH₂. In embodiments, R⁶ is —ONH₂. In embodiments, R⁶ is —NHC(O)NHNH₂. In embodiments, R⁶ is —NHC(O)NH₂. In embodiments, R⁶ is —NHSO₂H. In embodiments, R⁶ is —NHC(O)H. In embodiments, R⁶ is —NHC(O)OH. In embodiments, R⁶ is —NHOH. In embodiments, R⁶ is —OCCl₃. In embodiments, R⁶ is —OCF₃. In embodiments, R⁶ is —OCBr₃. In embodiments, R⁶ is —OCI₃. In embodiments, R⁶ is —OCHCl₂. In embodiments, R⁶ is —OCHBr₂. In embodiments, R⁶ is —OCHI₂. In embodiments, R⁶ is —OCHF₂. In embodiments, R⁶ is —OCH₂Cl. In embodiments, R⁶ is —OCH₂Br. In embodiments, R⁶ is —OCH₂I. In embodiments, R⁶ is —OCH₂F. In embodiments, R⁶ is —N₃. In embodiments, R⁶ is —CH₃. In embodiments, R⁶ is substituted or unsubstituted alkyl, or substituted or unsubstituted heteroalkyl.

In embodiments, R⁶ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁶ is hydrogen, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCH₂Cl, —OCH₂Br, —OCH₂I, —OCH₂F, —N₃, substituted or unsubstituted alkyl, substituted or unsubstituted heteroalkyl, or an amino acid side chain.

In embodiments, R⁶ is substituted (e.g., substituted with a substituent group, a size-limited substituent group, or lower substituent group) or unsubstituted alkyl. In embodiments, R⁶ is substituted (e.g., substituted with a substituent group, a size-limited substituent group, or lower substituent group) alkyl. In embodiments, R⁶ is unsubstituted alkyl. In embodiments, R⁶ is substituted or unsubstituted alkyl (e.g., C₁-C₅, C₁-C₆, C₁-C₄, or C₁-C₂). In embodiments, R⁶ is substituted alkyl (e.g., C₁-C₅, C₁-C₆, C₁-C₄, or C₁-C₂). In embodiments, R⁶ is unsubstituted alkyl (e.g., C₁-C₅, C₁-C₆, C₁-C₄, or C₁-C₂). In embodiments, R⁶ is an unsubstituted C₁-C₂ alkyl. In embodiments, R⁶ is an unsubstituted C₁-C₄alkyl. In embodiments, R⁶ is an unsubstituted C₁-C₆ alkyl. In embodiments, R⁶ is an unsubstituted C₁-C₅ alkyl. In embodiments, R⁶ is a substituted C₁-C₂ alkyl. In embodiments, R⁶ is a substituted C₁-C₄ alkyl. In embodiments, R⁶ is a substituted C₁-C₆ alkyl. In embodiments, R⁶ is a substituted C₁-C₈ alkyl. In embodiments, R⁶ is a substituted C₄ alkyl. In embodiments, R⁶ is an alkyl substituted with —NH₂, phenyl, indolyl, or imidazolyl. In embodiments, R⁶ is

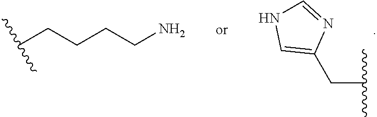 [SMILES: *CCCCN ; *Cc1c[nH]cn1]

In embodiments, R⁶ is substituted or unsubstituted alkyl, substituted or unsubstituted heteroalkyl. In embodiments, R⁶ is a sidechain of an amino acid (e.g., a sidechain of a non-natural amino acid or a sidechain natural amino acid). In embodiments, R⁶ is hydrogen,

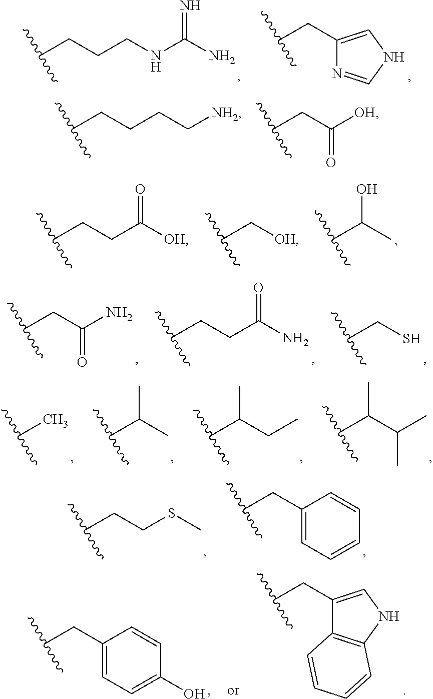

In embodiments, R⁶ is

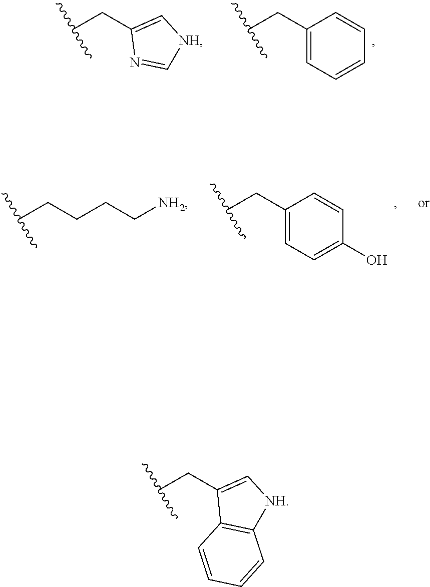 [SMILES: *Cc1c[nH]cn1 ; *Cc1ccccc1 ; *CCCCN ; *Cc1ccc(O)cc1 ; *Cc1c[nH]c2ccccc12]

In embodiments, R⁶ is:

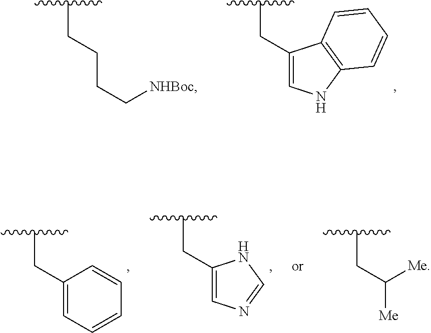 [SMILES: *CCCCNC(=O)OC(C)(C)C ; *Cc1c[nH]c2ccccc12 ; *Cc1ccccc1 ; *Cc1cnc[nH]1 ; *CC(C)C]

In embodiments, R⁶ is substituted (e.g., substituted with a substituent group, a size-limited substituent group, or lower substituent group) or unsubstituted heteroalkyl. In embodiments, R⁶ is substituted (e.g., substituted with a substituent group, a size-limited substituent group, or lower substituent group) heteroalkyl. In embodiments, R⁶ is unsubstituted heteroalkyl. In embodiments, R⁶ is substituted or unsubstituted heteroalkyl (e.g., 2 to 8 membered, 2 to 6 membered, 4 to 6 membered, 2 to 3 membered, or 4 to 5 membered). In embodiments, R⁶ is substituted heteroalkyl (e.g., 2 to 8 membered, 2 to 6 membered, 4 to 6 membered, 2 to 3 membered, or 4 to 5 membered). In embodiments, R⁶ is an unsubstituted heteroalkyl (e.g., 2 to 8 membered, 2 to 6 membered, 4 to 6 membered, 2 to 3 membered, or 4 to 5 membered).

In embodiments, R⁶ is an unsubstituted C₁-C₃ alkyl. In embodiments, R⁶ is an unsubstituted methyl.

In embodiments, R⁶ is R³⁹-substituted or unsubstituted alkyl (e.g., C₁-C₅ alkyl, C₁-C₆ alkyl, or C₁-C₄ alkyl), R³⁹-substituted or unsubstituted heteroalkyl (e.g., 2 to 8 membered heteroalkyl, 2 to 6 membered heteroalkyl, or 2 to 4 membered heteroalkyl), R³⁹-substituted or unsubstituted cycloalkyl (e.g., C₃-C₈ cycloalkyl, C₃-C₆ cycloalkyl, or C₅-C₆ cycloalkyl), R³⁹-substituted or unsubstituted heterocycloalkyl (e.g., 3 to 8 membered heterocycloalkyl, 3 to 6 membered heterocycloalkyl, or 5 to 6 membered heterocycloalkyl), R³⁹-substituted or unsubstituted aryl (e.g., C₆-C₁₀ aryl, C₁₀ aryl, or phenyl), or R³⁹-substituted or unsubstituted heteroaryl (e.g., 5 to 10 membered heteroaryl, 5 to 9 membered heteroaryl, or 5 to 6 membered heteroaryl).

In embodiments, R⁶ is R³⁹-substituted or unsubstituted alkyl (e.g., C₁-C₅ alkyl, C₁-C₆ alkyl, or C₁-C₄ alkyl). In embodiments, R⁶ is R³⁹-substituted alkyl (e.g., C₁-C₅ alkyl, C₁-C₆ alkyl, or C₁-C₄ alkyl). In embodiments, R⁶ is an unsubstituted alkyl (e.g., C₁-C₅ alkyl, C₁-C₆ alkyl, or C₁-C₄ alkyl).

In embodiments, R⁶ is R³⁹-substituted or unsubstituted heteroalkyl (e.g., 2 to 8 membered heteroalkyl, 2 to 6 membered heteroalkyl, or 2 to 4 membered heteroalkyl). In embodiments, R⁶ is R³⁹-substituted heteroalkyl (e.g., 2 to 8 membered heteroalkyl, 2 to 6 membered heteroalkyl, or 2 to 4 membered heteroalkyl). In embodiments, R⁶ is an unsubstituted heteroalkyl (e.g., 2 to 8 membered heteroalkyl, 2 to 6 membered heteroalkyl, or 2 to 4 membered heteroalkyl).

In embodiments, R⁶ is R³⁹-substituted or unsubstituted cycloalkyl (e.g., C₃-C₈ cycloalkyl, C₃-C₆ cycloalkyl, or C₅-C₆ cycloalkyl). In embodiments, R⁶ is R³⁹-substituted cycloalkyl (e.g., C₃-C₈ cycloalkyl, C₃-C₆ cycloalkyl, or C₅-C₆ cycloalkyl). In embodiments, R⁶ is an unsubstituted cycloalkyl (e.g., C₃-C₈ cycloalkyl, C₃-C₆ cycloalkyl, or C₅-C₆ cycloalkyl).

In embodiments, R⁶ is R³⁹-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁶ is R³⁹-substituted heterocycloalkyl (e.g., 3 to 8 membered heterocycloalkyl, 3 to 6 membered heterocycloalkyl, or 5 to 6 membered heterocycloalkyl). In embodiments, R⁶ is an unsubstituted heterocycloalkyl (e.g., 3 to 8 membered heterocycloalkyl, 3 to 6 membered heterocycloalkyl, or 5 to 6 membered heterocycloalkyl).

In embodiments, R⁶ is R³⁹-substituted or unsubstituted aryl (e.g., C₆-C₁₀ aryl, C₁₀ aryl, or phenyl). In embodiments, R⁶ is R³⁹-substituted aryl (e.g., C₆-C₁₀ aryl, C₁₀ aryl, or phenyl). In embodiments, R⁶ is an unsubstituted aryl (e.g., C₆-C₁₀ aryl, C₁₀ aryl, or phenyl).

In embodiments, R⁶ is R³⁹-substituted or unsubstituted heteroaryl (e.g., 5 to 10 membered heteroaryl, 5 to 9 membered heteroaryl, or 5 to 6 membered heteroaryl). In embodiments, R⁶ is R³⁹-substituted heteroaryl (e.g., 5 to 10 membered heteroaryl, 5 to 9 membered heteroaryl, or 5 to 6 membered heteroaryl). In embodiments, R⁶ is an unsubstituted heteroaryl (e.g., 5 to 10 membered heteroaryl, 5 to 9 membered heteroaryl, or 5 to 6 membered heteroaryl).

R³⁹ is independently oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R⁴-substituted or unsubstituted alkyl (e.g., C₁-C₅ alkyl, C₁-C₆ alkyl, or C₁-C₄ alkyl), R⁴⁰-substituted or unsubstituted heteroalkyl (e.g., 2 to 8 membered heteroalkyl, 2 to 6 membered heteroalkyl, or 2 to 4 membered heteroalkyl), R⁴⁰-substituted or unsubstituted cycloalkyl (e.g., C₃-C₈ cycloalkyl, C₃-C₆ cycloalkyl, or C₅-C₆ cycloalkyl), R⁴⁰-substituted or unsubstituted heterocycloalkyl (e.g., 3 to 8 membered heterocycloalkyl, 3 to 6 membered heterocycloalkyl, or 5 to 6 membered heterocycloalkyl), R⁴⁰-substituted or unsubstituted aryl (e.g., C₆-C₁₀ aryl, C₁₀ aryl, or phenyl), or R⁴⁰-substituted or unsubstituted heteroaryl (e.g., 5 to 10 membered heteroaryl, 5 to 9 membered heteroaryl, or 5 to 6 membered heteroaryl).

In embodiments, R³⁹ is R⁴⁰-substituted or unsubstituted alkyl (e.g., C₁-C₅ alkyl, C₁-C₆ alkyl, or C₁-C₄ alkyl), R⁴⁰-substituted or unsubstituted heteroalkyl (e.g., 2 to 8 membered heteroalkyl, 2 to 6 membered heteroalkyl, or 2 to 4 membered heteroalkyl), R⁴⁰-substituted or unsubstituted cycloalkyl (e.g., C₃-C₈ cycloalkyl, C₃-C₆ cycloalkyl, or C₅-C₆ cycloalkyl), R⁴-substituted or unsubstituted heterocycloalkyl (e.g., 3 to 8 membered heterocycloalkyl, 3 to 6 membered heterocycloalkyl, or 5 to 6 membered heterocycloalkyl), R⁴⁰-substituted or unsubstituted aryl (e.g., C₆-C₁₀ aryl, C₁₀ aryl, or phenyl), or R⁴⁰-substituted or unsubstituted heteroaryl (e.g., 5 to 10 membered heteroaryl, 5 to 9 membered heteroaryl, or 5 to 6 membered heteroaryl).

In embodiments, R³⁹ is R⁴⁰-substituted or unsubstituted alkyl (e.g., C₁-C₈ alkyl, C₁-C₆ alkyl, or C₁-C₄ alkyl). In embodiments, R³⁹ is R⁴⁰-substituted alkyl (e.g., C₁-C₈ alkyl, C₁-C₆ alkyl, or C₁-C₄ alkyl). In embodiments, R³⁹ is an unsubstituted alkyl (e.g., C₁-C₈ alkyl, C₁-C₆ alkyl, or C₁-C₄ alkyl).

In embodiments, R³⁹ is R⁴⁰-substituted or unsubstituted heteroalkyl (e.g., 2 to 8 membered heteroalkyl, 2 to 6 membered heteroalkyl, or 2 to 4 membered heteroalkyl). In embodiments, R³⁹ is R⁴⁰-substituted heteroalkyl (e.g., 2 to 8 membered heteroalkyl, 2 to 6 membered heteroalkyl, or 2 to 4 membered heteroalkyl). In embodiments, R³⁹ is an unsubstituted heteroalkyl (e.g., 2 to 8 membered heteroalkyl, 2 to 6 membered heteroalkyl, or 2 to 4 membered heteroalkyl).

In embodiments, R³⁹ is R⁴⁰-substituted or unsubstituted cycloalkyl (e.g., C₃—C cycloalkyl, C₃-C₆ cycloalkyl, or C₅-C₆ cycloalkyl). In embodiments, R³⁹ is R⁴⁰-substituted cycloalkyl (e.g., C₃-C₈ cycloalkyl, C₃-C₆ cycloalkyl, or C₅-C₆ cycloalkyl). In embodiments, R³⁹ is an unsubstituted cycloalkyl (e.g., C₃-C₈ cycloalkyl, C₃-C₆ cycloalkyl, or C₅-C₆ cycloalkyl).

In embodiments, R³⁹ is R⁴⁰-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R³⁹ is R⁴⁰-substituted heterocycloalkyl (e.g., 3 to 8 membered heterocycloalkyl, 3 to 6 membered heterocycloalkyl, or 5 to 6 membered heterocycloalkyl). In embodiments, R³⁹ is an unsubstituted heterocycloalkyl (e.g., 3 to 8 membered heterocycloalkyl, 3 to 6 membered heterocycloalkyl, or 5 to 6 membered heterocycloalkyl).

In embodiments, R³⁹ is R⁴⁰-substituted or unsubstituted aryl (e.g., C₆-C₁₀ aryl, C₁₀ aryl, or phenyl). In embodiments, R³⁹ is R⁴⁰-substituted aryl (e.g., C₆-C₁₀ aryl, C₁₀ aryl, or phenyl). In embodiments, R³⁹ is an unsubstituted aryl (e.g., C₆-C₁₀ aryl, C₁₀ aryl, or phenyl).

In embodiments, R³⁹ is R⁴⁰-substituted or unsubstituted heteroaryl (e.g., 5 to 10 membered heteroaryl, 5 to 9 membered heteroaryl, or 5 to 6 membered heteroaryl). In embodiments, R³⁹ is R⁴⁰-substituted heteroaryl (e.g., 5 to 10 membered heteroaryl, 5 to 9 membered heteroaryl, or 5 to 6 membered heteroaryl). In embodiments, R³⁹ is an unsubstituted heteroaryl (e.g., 5 to 10 membered heteroaryl, 5 to 9 membered heteroaryl, or 5 to 6 membered heteroaryl).

R⁴ is independently oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R⁴-substituted or unsubstituted alkyl (e.g., C₁-C₈ alkyl, C₁-C₆ alkyl, or C₁-C₄ alkyl), R⁴¹-substituted or unsubstituted heteroalkyl (e.g., 2 to 8 membered heteroalkyl, 2 to 6 membered heteroalkyl, or 2 to 4 membered heteroalkyl), R⁴¹-substituted or unsubstituted cycloalkyl (e.g., C₃-C₈ cycloalkyl, C₃-C₆ cycloalkyl, or C₅-C₆ cycloalkyl), R⁴¹-substituted or unsubstituted heterocycloalkyl (e.g., 3 to 8 membered heterocycloalkyl, 3 to 6 membered heterocycloalkyl, or 5 to 6 membered heterocycloalkyl), R⁴¹-substituted or unsubstituted aryl (e.g., C₆-C₁₀ aryl, C₁₀ aryl, or phenyl), or R⁴¹-substituted or unsubstituted heteroaryl (e.g., 5 to 10 membered heteroaryl, 5 to 9 membered heteroaryl, or 5 to 6 membered heteroaryl).

In embodiments, R⁴⁰ is R⁴¹-substituted or unsubstituted alkyl (e.g., C₁-C₈ alkyl, C₁-C₆ alkyl, or C₁-C₄ alkyl), R⁴¹-substituted or unsubstituted heteroalkyl (e.g., 2 to 8 membered heteroalkyl, 2 to 6 membered heteroalkyl, or 2 to 4 membered heteroalkyl), R⁴¹-substituted or unsubstituted cycloalkyl (e.g., C₃-C₈ cycloalkyl, C₃-C₆ cycloalkyl, or C₅-C₆ cycloalkyl), R⁴¹-substituted or unsubstituted heterocycloalkyl (e.g., 3 to 8 membered heterocycloalkyl, 3 to 6 membered heterocycloalkyl, or 5 to 6 membered heterocycloalkyl), R⁴¹-substituted or unsubstituted aryl (e.g., C₆-C₁₀ aryl, C₁₀ aryl, or phenyl), or R⁵⁰-substituted or unsubstituted heteroaryl (e.g., 5 to 10 membered heteroaryl, 5 to 9 membered heteroaryl, or 5 to 6 membered heteroaryl).

In embodiments, R⁴⁰ is R⁴¹-substituted or unsubstituted alkyl (e.g., C₁-C₈ alkyl, C₁-C₆ alkyl, or C₁-C₄ alkyl). In embodiments, R⁴⁰ is R⁴¹-substituted alkyl (e.g., C₁-C₅ alkyl, C₁-C₆ alkyl, or C₁-C₄ alkyl). In embodiments, R⁴⁰ is an unsubstituted alkyl (e.g., C₁-C₈ alkyl, C₁-C₆ alkyl, or C₁-C₄ alkyl).

In embodiments, R⁴⁰ is R⁴¹-substituted or unsubstituted heteroalkyl (e.g., 2 to 8 membered heteroalkyl, 2 to 6 membered heteroalkyl, or 2 to 4 membered heteroalkyl). In embodiments, R⁴⁰ is R⁴¹-substituted heteroalkyl (e.g., 2 to 8 membered heteroalkyl, 2 to 6 membered heteroalkyl, or 2 to 4 membered heteroalkyl). In embodiments, R⁴⁰ is an unsubstituted heteroalkyl (e.g., 2 to 8 membered heteroalkyl, 2 to 6 membered heteroalkyl, or 2 to 4 membered heteroalkyl).

In embodiments, R⁴⁰ is R⁴¹-substituted or unsubstituted cycloalkyl (e.g., C₃-C₈ cycloalkyl, C₃-C₆ cycloalkyl, or C₅-C₆ cycloalkyl). In embodiments, R⁴⁰ is R⁴¹-substituted cycloalkyl (e.g., C₃-C₈ cycloalkyl, C₃-C₆ cycloalkyl, or C₅-C₆ cycloalkyl). In embodiments, R⁴⁰ is an unsubstituted cycloalkyl (e.g., C₃-C₈ cycloalkyl, C₃-C₆ cycloalkyl, or C₅-C₆ cycloalkyl).

In embodiments, R⁴ is R⁴¹-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁴ is R⁴¹-substituted heterocycloalkyl (e.g., 3 to 8 membered heterocycloalkyl, 3 to 6 membered heterocycloalkyl, or 5 to 6 membered heterocycloalkyl). In embodiments, R⁴ is an unsubstituted heterocycloalkyl (e.g., 3 to 8 membered heterocycloalkyl, 3 to 6 membered heterocycloalkyl, or 5 to 6 membered heterocycloalkyl).

In embodiments, R⁴ is R⁴¹-substituted or unsubstituted aryl (e.g., C₆-C₁₀ aryl, C₁₀ aryl, or phenyl). In embodiments, R⁴ is R⁴¹-substituted aryl (e.g., C₆-C₁₀ aryl, C₁₀ aryl, or phenyl). In embodiments, R⁴ is an unsubstituted aryl (e.g., C₆-C₁₀ aryl, C₁₀ aryl, or phenyl).

In embodiments, R⁴ is R⁴¹-substituted or unsubstituted heteroaryl (e.g., 5 to 10 membered heteroaryl, 5 to 9 membered heteroaryl, or 5 to 6 membered heteroaryl). In embodiments, R⁴⁰ is R⁴¹-substituted heteroaryl (e.g., 5 to 10 membered heteroaryl, 5 to 9 membered heteroaryl, or 5 to 6 membered heteroaryl). In embodiments, R⁴ is an unsubstituted heteroaryl (e.g., 5 to 10 membered heteroaryl, 5 to 9 membered heteroaryl, or 5 to 6 membered heteroaryl).

In embodiments, R⁶ and R⁷ are joined to form a substituted or unsubstituted heterocycloalkyl or heteroaryl. In embodiments, R⁶ and R⁷ are joined to form a substituted or unsubstituted 3 to 6 membered heterocycloalkyl, or substituted or unsubstituted 5 to 6 membered heteroaryl. In embodiments, R⁶ and R⁷ are joined to form a substituted or unsubstituted 3 to 6 membered heterocycloalkyl. In embodiments, R⁶ and R⁷ are joined to form a substituted or unsubstituted pyrrolidinyl or substituted or unsubstituted 2,3-dihydropyrrolyl. In embodiments, R⁶ and R⁷ are joined to form a substituted pyrrolidinyl or substituted 2,3-dihydropyrrolyl. In embodiments, R⁶ and R⁷ are joined to form a substituted pyrrolidinyl. In embodiments, R⁶ and R⁷ are joined to form a substituted 2,3-dihydropyrrolyl. In embodiments, R⁶ and R⁷ are joined to form an unsubstituted pyrrolidinyl. In embodiments, R⁶ and R⁷ are joined to form an unsubstituted 2,3-dihydropyrrolyl.

In embodiments, R⁶ and R⁷ may optionally be joined to form an unsubstituted heterocycloalkyl or unsubstituted heteroaryl.

In embodiments, R⁶ and R⁷ are joined to forma substituted or unsubstituted heterocycloalkyl or substituted or unsubstituted heteroaryl, which may be referred to herein as Ring B, shown below:

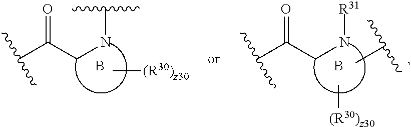

wherein the

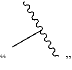

or moiety attached to Ring B is a bond to the carbonyl moiety on the remainder of the compound, and z30 is an integer from 0 to 6.

R³⁰ is halogen, —CCl₃, —CBr₃, —CF₃, —CI₃, CHCl₂, —CHBr₂, —CHF₂, —CHI₂, —CH₂CL, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl). In embodiments, R³⁰ is halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl (e.g., C₁-C₈ alkyl, C₁-C₆ alkyl, or C₁-C₄ alkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl)

In embodiments, R³⁰ is R⁵⁸-substituted or unsubstituted alkyl (e.g., C₁-C₈ alkyl, C₁-C₆ alkyl, or C₁-C₄ alkyl), R⁵⁸-substituted or unsubstituted heteroalkyl (e.g., 2 to 8 membered heteroalkyl, 2 to 6 membered heteroalkyl, or 2 to 4 membered heteroalkyl), R⁵⁸-substituted or unsubstituted cycloalkyl (e.g., C₃-C₈ cycloalkyl, C₃-C₆ cycloalkyl, or C₅-C₆ cycloalkyl), R⁵⁸-substituted or unsubstituted heterocycloalkyl (e.g., 3 to 8 membered heterocycloalkyl, 3 to 6 membered heterocycloalkyl, or 5 to 6 membered heterocycloalkyl), R⁵⁸-substituted or unsubstituted aryl (e.g., C₆-C₁₀ aryl, C₁₀ aryl, or phenyl), or R⁵⁸-substituted or unsubstituted heteroaryl (e.g., 5 to 10 membered heteroaryl, 5 to 9 membered heteroaryl, or 5 to 6 membered heteroaryl).

In embodiments, R³⁰ is R⁵⁸-substituted or unsubstituted alkyl (e.g., C₁-C₈ alkyl, C₁-C₆ alkyl, or C₁-C₄ alkyl). In embodiments, R³⁰ is R⁵⁸-substituted alkyl (e.g., C₁-C₈ alkyl, C₁-C₆ alkyl, or C₁-C₄ alkyl). In embodiments, R³⁰ is an unsubstituted alkyl (e.g., C₁-C₈ alkyl, C₁-C₆ alkyl, or C₁-C₄ alkyl).

In embodiments, R³⁰ is R⁵⁸-substituted or unsubstituted heteroalkyl (e.g., 2 to 8 membered heteroalkyl, 2 to 6 membered heteroalkyl, or 2 to 4 membered heteroalkyl). In embodiments, R³⁰ is R⁵⁸-substituted heteroalkyl (e.g., 2 to 8 membered heteroalkyl, 2 to 6 membered heteroalkyl, or 2 to 4 membered heteroalkyl). In embodiments, R³⁰ is an unsubstituted heteroalkyl (e.g., 2 to 8 membered heteroalkyl, 2 to 6 membered heteroalkyl, or 2 to 4 membered heteroalkyl).

In embodiments, R³⁰ is R⁵⁸-substituted or unsubstituted cycloalkyl (e.g., C₃-C₈ cycloalkyl, C₃-C₆ cycloalkyl, or C₅-C₆ cycloalkyl). In embodiments, R³⁰ is R⁵⁸-substituted cycloalkyl (e.g., C₃-C₈ cycloalkyl, C₃-C₆ cycloalkyl, or C₅-C₆ cycloalkyl). In embodiments, R³⁰ is an unsubstituted cycloalkyl (e.g., C₃-C₈ cycloalkyl, C₃-C₆ cycloalkyl, or C₅-C₆ cycloalkyl).

In embodiments, R³⁰ is R⁵⁸-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R³⁰ is R⁵⁸-substituted heterocycloalkyl (e.g., 3 to 8 membered heterocycloalkyl, 3 to 6 membered heterocycloalkyl, or 5 to 6 membered heterocycloalkyl). In embodiments, R³⁰ is an unsubstituted heterocycloalkyl (e.g., 3 to 8 membered heterocycloalkyl, 3 to 6 membered heterocycloalkyl, or 5 to 6 membered heterocycloalkyl).

In embodiments, R³⁰ is R⁵⁸-substituted or unsubstituted aryl (e.g., C₆-C₁₀ aryl, C₁₀ aryl, or phenyl). In embodiments, R³⁰ is R⁵⁸-substituted aryl (e.g., C₆-C₁₀ aryl, C₁₀ aryl, or phenyl). In embodiments, R³⁰ is an unsubstituted aryl (e.g., C₆-C₁₀ aryl, C₁₀ aryl, or phenyl).

In embodiments, R³⁰ is R⁵⁸-substituted or unsubstituted heteroaryl (e.g., 5 to 10 membered heteroaryl, 5 to 9 membered heteroaryl, or 5 to 6 membered heteroaryl). In embodiments, R³⁰ is R⁵⁸-substituted heteroaryl (e.g., 5 to 10 membered heteroaryl, 5 to 9 membered heteroaryl, or 5 to 6 membered heteroaryl). In embodiments, R³⁰ is an unsubstituted heteroaryl (e.g., 5 to 10 membered heteroaryl, 5 to 9 membered heteroaryl, or 5 to 6 membered heteroaryl).

R³¹ is a leaving group, protecting group, hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, R³¹ is R⁵⁹-substituted or unsubstituted alkyl (e.g., C₁-C₈ alkyl, C₁-C₆ alkyl, or C₁-C₄ alkyl), R⁵⁹-substituted or unsubstituted heteroalkyl (e.g., 2 to 8 membered heteroalkyl, 2 to 6 membered heteroalkyl, or 2 to 4 membered heteroalkyl), R⁵⁹-substituted or unsubstituted cycloalkyl (e.g., C₃-C₈ cycloalkyl, C₃-C₆ cycloalkyl, or C₅-C₆ cycloalkyl), R⁵⁹-substituted or unsubstituted heterocycloalkyl (e.g., 3 to 8 membered heterocycloalkyl, 3 to 6 membered heterocycloalkyl, or 5 to 6 membered heterocycloalkyl), R⁵⁹-substituted or unsubstituted aryl (e.g., C₆-C₁₀ aryl, C₁₀ aryl, or phenyl), or R⁵⁹-substituted or unsubstituted heteroaryl (e.g., 5 to 10 membered heteroaryl, 5 to 9 membered heteroaryl, or 5 to 6 membered heteroaryl).

In embodiments, R³¹ is R⁵⁹-substituted or unsubstituted alkyl (e.g., C₁-C₈ alkyl, C₁-C₆ alkyl, or C₁-C₄ alkyl). In embodiments, R³¹ is R⁵⁹-substituted alkyl (e.g., C₁-C₈ alkyl, C₁-C₆ alkyl, or C₁-C₄ alkyl). In embodiments, R³¹ is an unsubstituted alkyl (e.g., C₁-C₈ alkyl, C₁-C₆ alkyl, or C₁-C₄ alkyl).

In embodiments, R³¹ is R⁵⁹-substituted or unsubstituted heteroalkyl (e.g., 2 to 8 membered heteroalkyl, 2 to 6 membered heteroalkyl, or 2 to 4 membered heteroalkyl). In embodiments, R³¹ is R⁵⁹-substituted heteroalkyl (e.g., 2 to 8 membered heteroalkyl, 2 to 6 membered heteroalkyl, or 2 to 4 membered heteroalkyl). In embodiments, R³¹ is an unsubstituted heteroalkyl (e.g., 2 to 8 membered heteroalkyl, 2 to 6 membered heteroalkyl, or 2 to 4 membered heteroalkyl).

In embodiments, R³¹ is R⁵⁹-substituted or unsubstituted cycloalkyl (e.g., C₃-C₈ cycloalkyl, C₃-C₆ cycloalkyl, or C₅-C₆ cycloalkyl). In embodiments, R³¹ is R⁵⁹-substituted cycloalkyl (e.g., C₃-C₈ cycloalkyl, C₃-C₆ cycloalkyl, or C₅-C₆ cycloalkyl). In embodiments, R³¹ is an unsubstituted cycloalkyl (e.g., C₃-C₈ cycloalkyl, C₃-C₆ cycloalkyl, or C₅-C₆ cycloalkyl).

In embodiments, R³¹ is R⁵⁹-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R³¹ is R⁵⁹-substituted heterocycloalkyl (e.g., 3 to 8 membered heterocycloalkyl, 3 to 6 membered heterocycloalkyl, or 5 to 6 membered heterocycloalkyl). In embodiments, R³¹ is an unsubstituted heterocycloalkyl (e.g., 3 to 8 membered heterocycloalkyl, 3 to 6 membered heterocycloalkyl, or 5 to 6 membered heterocycloalkyl).

In embodiments, R³¹ is R⁵⁹-substituted or unsubstituted aryl (e.g., C₆-C₁₀ aryl, C₁₀ aryl, or phenyl). In embodiments, R³¹ is R⁵⁹-substituted aryl (e.g., C₆-C₁₀ aryl, C₁₀ aryl, or phenyl). In embodiments, R³¹ is an unsubstituted aryl (e.g., C₆-C₁₀ aryl, C₁₀ aryl, or phenyl).

In embodiments, R³¹ is R⁵⁹-substituted or unsubstituted heteroaryl (e.g., 5 to 10 membered heteroaryl, 5 to 9 membered heteroaryl, or 5 to 6 membered heteroaryl). In embodiments, R³¹ is R⁵⁹-substituted heteroaryl (e.g., 5 to 10 membered heteroaryl, 5 to 9 membered heteroaryl, or 5 to 6 membered heteroaryl). In embodiments, R³¹ is an unsubstituted heteroaryl (e.g., 5 to 10 membered heteroaryl, 5 to 9 membered heteroaryl, or 5 to 6 membered heteroaryl).

In embodiments, R⁶ and R⁷ are joined to form a 4 to 8 membered heterocycloalkyl. In embodiments, R⁶ and R⁷ are joined to form a 4 membered heterocycloalkyl. In embodiments, R⁶ and R⁷ are joined to form a 5 membered heterocycloalkyl. In embodiments, R⁶ and R⁷ are joined to form a 6 membered heterocycloalkyl. In embodiments, R⁶ and R⁷ are joined to form a 7 membered heterocycloalkyl. In embodiments, R⁶ and R⁷ are joined to form an 8 membered heterocycloalkyl. In embodiments, R⁶ and R⁷ are joined to form a 4 to 6 membered heterocycloalkyl. In embodiments, R⁶ and R⁷ are joined to form azetidinyl.

In embodiments, R⁶ and R⁷ are joined to form a substituted or unsubstituted heterocycloalkyl. In embodiments, R⁶ and R⁷ are joined to form a substituted heterocycloalkyl. In embodiments, R⁶ and R⁷ are joined to form an unsubstituted heterocycloalkyl. In embodiments, R⁶ and R⁷ are joined to form a substituted or unsubstituted 3 to 10 membered heterocycloalkyl. In embodiments, R⁶ and R⁷ are joined to form a substituted 3 to 10 membered heterocycloalkyl. In embodiments, R⁶ and R⁷ are joined to form an unsubstituted 3 to 10 membered heterocycloalkyl. In embodiments, R⁶ and R⁷ are joined to form a R³⁰-substituted or unsubstituted 5 to 10 membered heterocycloalkyl. In embodiments, R⁶ and R⁷ are joined to form a R³⁰-substituted 5 to 10 membered heterocycloalkyl. In embodiments, R⁶ and R⁷ are joined to form an unsubstituted 5 to 10 membered heterocycloalkyl.

In embodiments, R⁶ and R⁷ are joined to form a substituted or unsubstituted 3 membered heterocycloalkyl. In embodiments, R⁶ and R⁷ are joined to form a substituted or unsubstituted 4 membered heterocycloalkyl. In embodiments, R⁶ and R⁷ are joined to form a substituted or unsubstituted 5 membered heterocycloalkyl. In embodiments, R⁶ and R⁷ are joined to form a substituted or unsubstituted 6 membered heterocycloalkyl. In embodiments, R⁶ and R⁷ are joined to form a substituted 3 membered heterocycloalkyl. In embodiments, R⁶ and R⁷ are joined to form a substituted 4 membered heterocycloalkyl. In embodiments, R⁶ and R⁷ are joined to form a substituted 5 membered heterocycloalkyl. In embodiments, R⁶ and R⁷ are joined to form a substituted 6 membered heterocycloalkyl. In embodiments, R⁶ and R⁷ are joined to form an unsubstituted 3 membered heterocycloalkyl. In embodiments, R⁶ and R⁷ are joined to form an unsubstituted 4 membered heterocycloalkyl. In embodiments, R⁶ and R⁷ are joined to form an unsubstituted 5 membered heterocycloalkyl. In embodiments, R⁶ and R⁷ are joined to form an unsubstituted 6 membered heterocycloalkyl.

In embodiments, R⁶ and R⁷ are joined to form a substituted or unsubstituted aziridinyl, azirinyl, azetidinyl, dihydroazetyl, diazetidinyl, azetyl, pyrrolidinyl, pyrrolinyl, pyrrolyl, pyrazolidinyl, imidazolidinyl, pyrazolinyl, pyrazolyl, thiazolidinyl, thiazolyl, isothiazolyl, piperidinyl, piperazinyl, morpholinyl, oxazinyl, thiomorpholinyl, thiazinyl, decahydroquinolinyl, dihydroazepinyl, azepanyl, or azocanyl. In embodiments, R⁶ and R⁷ are joined to form a substituted aziridinyl, azirinyl, azetidinyl, dihydroazetyl, diazetidinyl, azetyl, pyrrolidinyl, pyrrolinyl, pyrrolyl, pyrazolidinyl, imidazolidinyl, pyrazolinyl, pyrazolyl, thiazolidinyl, thiazolyl, isothiazolyl, piperidinyl, piperazinyl, morpholinyl, oxazinyl, thiomorpholinyl, thiazinyl, decahydroquinolinyl, dihydroazepinyl, azepanyl, or azocanyl. In embodiments, R⁶ and R⁷ are joined to form an unsubstituted aziridinyl, azirinyl, azetidinyl, dihydroazetyl, diazetidinyl, azetyl, pyrrolidinyl, pyrrolinyl, pyrrolyl, pyrazolidinyl, imidazolidinyl, pyrazolinyl, pyrazolyl, thiazolidinyl, thiazolyl, isothiazolyl, piperidinyl, piperazinyl, morpholinyl, oxazinyl, thiomorpholinyl, thiazinyl, decahydroquinolinyl, dihydroazepinyl, azepanyl, or azocanyl.

In embodiments, R⁷ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —CCl₃, —COOH, —CH₂COOH, —CONH₂, —OH, —SH, —NO₂, —NH₂, —NHNH₂, —ONH₂, or —NHC(O)NHNH₂. In embodiments, R⁷ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁷ is hydrogen, substituted or unsubstituted alkyl, or substituted or unsubstituted heteroalkyl. In embodiments, R⁷ is hydrogen.

In embodiments, R⁷ is hydrogen, —CH₂COOH, —CONH₂, —OH, —SH, —NO₂, —NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁷ is substituted (e.g., substituted with a substituent group, a size-limited substituent group, or lower substituent group) or unsubstituted alkyl. In embodiments, R⁷ is substituted (e.g., substituted with a substituent group, a size-limited substituent group, or lower substituent group) alkyl. In embodiments, R⁷ is unsubstituted alkyl. In embodiments, R⁷ is substituted or unsubstituted alkyl (e.g., C₁-C₈, C₁-C₆, C₁-C₄, or C₁-C₂). In embodiments, R⁷ is substituted alkyl (e.g., C₁-C₈, C₁-C₆, C₁-C₄, or C₁-C₂). In embodiments, R⁷ is unsubstituted alkyl (e.g., C₁-C₈, C₁-C₆, C₁-C₄, or C₁-C₂).

In embodiments, R⁷ is substituted (e.g., substituted with a substituent group, a size-limited substituent group, or lower substituent group) or unsubstituted heteroalkyl. In embodiments, R⁷ is substituted (e.g., substituted with a substituent group, a size-limited substituent group, or lower substituent group) heteroalkyl. In embodiments, R⁷ is unsubstituted heteroalkyl. In embodiments, R⁷ is substituted or unsubstituted heteroalkyl (e.g., 2 to 8 membered, 2 to 6 membered, 4 to 6 membered, 2 to 3 membered, or 4 to 5 membered). In embodiments, R⁷ is substituted heteroalkyl (e.g., 2 to 8 membered, 2 to 6 membered, 4 to 6 membered, 2 to 3 membered, or 4 to 5 membered). In embodiments, R⁷ is an unsubstituted heteroalkyl (e.g., 2 to 8 membered, 2 to 6 membered, 4 to 6 membered, 2 to 3 membered, or 4 to 5 membered).

In embodiments, R⁷ is substituted (e.g., substituted with a substituent group, a size-limited substituent group, or lower substituent group) or unsubstituted cycloalkyl. In embodiments, R⁷ is substituted (e.g., substituted with a substituent group, a size-limited substituent group, or lower substituent group) cycloalkyl. In embodiments, R⁷ is an unsubstituted cycloalkyl. In embodiments, R⁷ is substituted or unsubstituted cycloalkyl (e.g., C₃-C₈, C₃-C₆, C₄-C₆, or C₅-C₆). In embodiments, R⁷ is substituted cycloalkyl (e.g., C₃-C₈, C₃-C₆, C₄-C₆, or C₅-C₆). In embodiments, R⁷ is unsubstituted cycloalkyl (e.g., C₃-C₈, C₃-C₆, C₄-C₆, or C₅-C₆).

In embodiments, R⁷ is substituted (e.g., substituted with a substituent group, a size-limited substituent group, or lower substituent group) or unsubstituted heterocycloalkyl. In embodiments, R⁷ is substituted (e.g., substituted with a substituent group, a size-limited substituent group, or lower substituent group) heterocycloalkyl. In embodiments, R⁷ is an unsubstituted heterocycloalkyl. In embodiments, R⁷ is substituted or unsubstituted heterocycloalkyl (e.g., 3 to 8 membered, 3 to 6 membered, 4 to 6 membered, 4 to 5 membered, or 5 to 6 membered). In embodiments, R⁷ is substituted heterocycloalkyl (e.g., 3 to 8 membered, 3 to 6 membered, 4 to 6 membered, 4 to 5 membered, or 5 to 6 membered). In embodiments, R⁷ an unsubstituted heterocycloalkyl (e.g., 3 to 8 membered, 3 to 6 membered, 4 to 6 membered, 4 to 5 membered, or 5 to 6 membered).

In embodiments, R⁷ is substituted (e.g., substituted with a substituent group, a size-limited substituent group, or lower substituent group) or unsubstituted aryl. In embodiments, R⁷ is substituted (e.g., substituted with a substituent group, a size-limited substituent group, or lower substituent group) aryl. In embodiments, R⁷ is an unsubstituted aryl. In embodiments, R⁷ is substituted or unsubstituted aryl (e.g., C₆-C₁₀ or phenyl). In embodiments, R⁷ is substituted aryl (e.g., C₆-C₁₀ or phenyl). In embodiments, R⁷ is an unsubstituted aryl (e.g., C₆-C₁₀ or phenyl).

In embodiments, R⁷ is substituted (e.g., substituted with a substituent group, a size-limited substituent group, or lower substituent group) or unsubstituted heteroaryl. In embodiments, R⁷ is substituted (e.g., substituted with a substituent group, a size-limited substituent group, or lower substituent group) heteroaryl. In embodiments, R⁷ is an unsubstituted heteroaryl. In embodiments, R⁷ is substituted or unsubstituted heteroaryl (e.g., 5 to 10 membered, 5 to 9 membered, or 5 to 6 membered). In embodiments, R⁷ is substituted heteroaryl (e.g., 5 to 10 membered, 5 to 9 membered, or 5 to 6 membered). In embodiments, R⁷ is an unsubstituted heteroaryl (e.g., 5 to 10 membered, 5 to 9 membered, or 5 to 6 membered).

In embodiments, R⁷ is hydrogen or substituted or unsubstituted C₁-C₆ alkyl. In embodiments, R⁷ is hydrogen.

In embodiments, R⁷ is R⁴²-substituted or unsubstituted alkyl (e.g., C₁-C₈ alkyl, C₁-C₇ alkyl, or C₁-C₄ alkyl), R⁴²-substituted or unsubstituted heteroalkyl (e.g., 2 to 8 membered heteroalkyl, 2 to 7 membered heteroalkyl, or 2 to 4 membered heteroalkyl), R⁴²-substituted or unsubstituted cycloalkyl (e.g., C₃-C₈ cycloalkyl, C₃-C₇ cycloalkyl, or C₅-C₇ cycloalkyl), R⁴²-substituted or unsubstituted heterocycloalkyl (e.g., 3 to 8 membered heterocycloalkyl, 3 to 7 membered heterocycloalkyl, or 5 to 7 membered heterocycloalkyl), R⁴²-substituted or unsubstituted aryl (e.g., C₇-C₁₀ aryl, C₁₀ aryl, or phenyl), or R⁴²-substituted or unsubstituted heteroaryl (e.g., 5 to 10 membered heteroaryl, 5 to 9 membered heteroaryl, or 5 to 7 membered heteroaryl).

In embodiments, R⁷ is R⁴²-substituted or unsubstituted alkyl (e.g., C₁-C₈ alkyl, C₁-C₇ alkyl, or C₁-C₄ alkyl). In embodiments, R⁷ is R⁴²-substituted alkyl (e.g., C₁-C₈ alkyl, C₁-C₇ alkyl, or C₁-C₄ alkyl). In embodiments, R⁷ is an unsubstituted alkyl (e.g., C₁-C₈ alkyl, C₁-C₇ alkyl, or C₁-C₄ alkyl).

In embodiments, R⁷ is R⁴²-substituted or unsubstituted heteroalkyl (e.g., 2 to 8 membered heteroalkyl, 2 to 7 membered heteroalkyl, or 2 to 4 membered heteroalkyl). In embodiments, R⁷ is R⁴²-substituted heteroalkyl (e.g., 2 to 8 membered heteroalkyl, 2 to 7 membered heteroalkyl, or 2 to 4 membered heteroalkyl). In embodiments, R⁷ is an unsubstituted heteroalkyl (e.g., 2 to 8 membered heteroalkyl, 2 to 7 membered heteroalkyl, or 2 to 4 membered heteroalkyl).

In embodiments, R⁷ is R⁴²-substituted or unsubstituted cycloalkyl (e.g., C₃-C₈ cycloalkyl, C₃-C₇ cycloalkyl, or C₅-C₇ cycloalkyl). In embodiments, R⁷ is R⁴²-substituted cycloalkyl (e.g., C₃-C₈ cycloalkyl, C₃-C₇ cycloalkyl, or C₅-C₇ cycloalkyl). In embodiments, R⁷ is an unsubstituted cycloalkyl (e.g., C₃-C₈ cycloalkyl, C₃-C₇ cycloalkyl, or C₅-C₇ cycloalkyl).

In embodiments, R⁷ is R⁴²-substituted or unsubstituted heterocycloalkyl (e.g., 3 to 8 membered heterocycloalkyl, 3 to 7 membered heterocycloalkyl, or 5 to 7 membered heterocycloalkyl). In embodiments, R⁷ is R⁴²-substituted heterocycloalkyl (e.g., 3 to 8 membered heterocycloalkyl, 3 to 7 membered heterocycloalkyl, or 5 to 7 membered heterocycloalkyl). In embodiments, R⁷ is an unsubstituted heterocycloalkyl (e.g., 3 to 8 membered heterocycloalkyl, 3 to 7 membered heterocycloalkyl, or 5 to 7 membered heterocycloalkyl).

In embodiments, R⁷ is R⁴²-substituted or unsubstituted aryl (e.g., C₇-C₁₀ aryl, C₁₀ aryl, or phenyl). In embodiments, R⁷ is R⁴²-substituted aryl (e.g., C₇-C₁₀ aryl, C₁₀ aryl, or phenyl). In embodiments, R⁷ is an unsubstituted aryl (e.g., C₇-C₁₀ aryl, C₁₀ aryl, or phenyl).

In embodiments, R⁷ is R⁴²-substituted or unsubstituted heteroaryl (e.g., 5 to 10 membered heteroaryl, 5 to 9 membered heteroaryl, or 5 to 7 membered heteroaryl). In embodiments, R⁷ is R⁴²-substituted heteroaryl (e.g., 5 to 10 membered heteroaryl, 5 to 9 membered heteroaryl, or 5 to 7 membered heteroaryl). In embodiments, R⁷ is an unsubstituted heteroaryl (e.g., 5 to 10 membered heteroaryl, 5 to 9 membered heteroaryl, or 5 to 7 membered heteroaryl).

In embodiments, R⁸ is oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, or —N₃. In embodiments, R⁸ is hydrogen. In embodiments, R⁸ is oxo. In embodiments, R⁸ is halogen. In embodiments, R⁸ is —CCl₃. In embodiments, R⁸ is —CBr₃. In embodiments, R⁸ is —CF₃. In embodiments, R⁸ is —CI₃. In embodiments, R⁸ is CHCl₂. In embodiments, R⁸ is —CHBr₂. In embodiments, R⁸ is —CHF₂. In embodiments, R⁸ is —CHI₂. In embodiments, R⁸ is —CH₂C₁. In embodiments, R⁸ is —CH₂Br. In embodiments, R⁸ is —CH₂F. In embodiments, R⁸ is —CH₂I. In embodiments, R⁸ is —CN. In embodiments, R⁸ is —OH. In embodiments, R⁸ is —NH₂. In embodiments, R⁸ is —COOH. In embodiments, R⁸ is —CONH₂. In embodiments, R⁸ is —NO₂. In embodiments, R⁸ is —SH. In embodiments, R⁸ is —SO₃H. In embodiments, R⁸ is —SO₄H. In embodiments, R⁸ is —SO₂NH₂. In embodiments, R⁸ is —NHNH₂. In embodiments, R⁸ is —ONH₂. In embodiments, R⁸ is —NHC(O)NHNH₂. In embodiments, R⁸ is —NHC(O)NH₂. In embodiments, R⁸ is —NHSO₂H. In embodiments, R⁸ is —NHC(O)H. In embodiments, R⁸ is —NHC(O)OH. In embodiments, R⁸ is —NHOH. In embodiments, R⁸ is —OCCI₃. In embodiments, R⁸ is —OCF₃. In embodiments, R⁸ is —OCBr₃. In embodiments, R⁸ is —OCI₃. In embodiments, R⁸ is —OCHCl₂. In embodiments, R⁸ is —OCHBr₂. In embodiments, R⁸ is —OCHI₂. In embodiments, R⁸ is —OCHF₂. In embodiments, R⁸ is —OCH₂C₁. In embodiments, R⁸ is —OCH₂Br. In embodiments, R⁸ is —OCH₂I. In embodiments, R⁸ is —OCH₂F. In embodiments, R⁸ is —N₃.

In embodiments, R⁸ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁸ is substituted (e.g., substituted with a substituent group, a size-limited substituent group, or lower substituent group) or unsubstituted alkyl. In embodiments, R⁸ is substituted (e.g., substituted with a substituent group, a size-limited substituent group, or lower substituent group) alkyl. In embodiments, R⁸ is unsubstituted alkyl. In embodiments, R⁸ is substituted or unsubstituted alkyl (e.g., C₁-C₈, C₁-C₆, C₁-C₄, or C₁-C₂). In embodiments, R⁸ is substituted alkyl (e.g., C₁-C₈, C₁-C₆, C₁-C₄, or C₁-C₂). In embodiments, R⁸ is unsubstituted alkyl (e.g., C₁-C₈, C₁-C₆, C₁-C₄, or C₁-C₂).

In embodiments, R⁸ is substituted (e.g., substituted with a substituent group, a size-limited substituent group, or lower substituent group) or unsubstituted heteroalkyl. In embodiments, R⁸ is substituted (e.g., substituted with a substituent group, a size-limited substituent group, or lower substituent group) heteroalkyl. In embodiments, R⁸ is unsubstituted heteroalkyl. In embodiments, R⁸ is substituted or unsubstituted heteroalkyl (e.g., 2 to 8 membered, 2 to 6 membered, 4 to 6 membered, 2 to 3 membered, or 4 to 5 membered). In embodiments, R⁸ is substituted heteroalkyl (e.g., 2 to 8 membered, 2 to 6 membered, 4 to 6 membered, 2 to 3 membered, or 4 to 5 membered). In embodiments, R⁸ is an unsubstituted heteroalkyl (e.g., 2 to 8 membered, 2 to 6 membered, 4 to 6 membered, 2 to 3 membered, or 4 to 5 membered).

In embodiments, R⁸ is substituted (e.g., substituted with a substituent group, a size-limited substituent group, or lower substituent group) or unsubstituted cycloalkyl. In embodiments, R⁸ is substituted (e.g., substituted with a substituent group, a size-limited substituent group, or lower substituent group) cycloalkyl. In embodiments, R⁸ is an unsubstituted cycloalkyl. In embodiments, R⁸ is substituted or unsubstituted cycloalkyl (e.g., C₃-C₈, C₃-C₆, C₄-C₆, or C₅-C₆). In embodiments, R⁸ is substituted cycloalkyl (e.g., C₃-C₈, C₃-C₆, C₄-C₆, or C₅-C₆). In embodiments, R⁸ is unsubstituted cycloalkyl (e.g., C₃-C₈, C₃-C₆, C₄-C₆, or C₅-C₆).

In embodiments, R⁸ is substituted (e.g., substituted with a substituent group, a size-limited substituent group, or lower substituent group) or unsubstituted heterocycloalkyl. In embodiments, R⁸ is substituted (e.g., substituted with a substituent group, a size-limited substituent group, or lower substituent group) heterocycloalkyl. In embodiments, R⁸ is an unsubstituted heterocycloalkyl. In embodiments, R⁸ is substituted or unsubstituted heterocycloalkyl (e.g., 3 to 8 membered, 3 to 6 membered, 4 to 6 membered, 4 to 5 membered, or 5 to 6 membered). In embodiments, R⁸ is substituted heterocycloalkyl (e.g., 3 to 8 membered, 3 to 6 membered, 4 to 6 membered, 4 to 5 membered, or 5 to 6 membered). In embodiments, R⁸ an unsubstituted heterocycloalkyl (e.g., 3 to 8 membered, 3 to 6 membered, 4 to 6 membered, 4 to 5 membered, or 5 to 6 membered).

In embodiments, R⁸ is substituted (e.g., substituted with a substituent group, a size-limited substituent group, or lower substituent group) or unsubstituted aryl. In embodiments, R⁸ is substituted (e.g., substituted with a substituent group, a size-limited substituent group, or lower substituent group) aryl. In embodiments, R⁸ is an unsubstituted aryl. In embodiments, R⁸ is substituted or unsubstituted aryl (e.g., C₆-C₁₀ or phenyl). In embodiments, R⁸ is substituted aryl (e.g., C₆-C₁₀ or phenyl). In embodiments, R⁸ is an unsubstituted aryl (e.g., C₆-C₁₀ or phenyl).

In embodiments, R⁸ is substituted (e.g., substituted with a substituent group, a size-limited substituent group, or lower substituent group) or unsubstituted heteroaryl. In embodiments, R⁸ is substituted (e.g., substituted with a substituent group, a size-limited substituent group, or lower substituent group) heteroaryl. In embodiments, R⁸ is an unsubstituted heteroaryl. In embodiments, R⁸ is substituted or unsubstituted heteroaryl (e.g., 5 to 10 membered, 5 to 9 membered, or 5 to 6 membered). In embodiments, R⁸ is substituted heteroaryl (e.g., 5 to 10 membered, 5 to 9 membered, or 5 to 6 membered). In embodiments, R⁸ is an unsubstituted heteroaryl (e.g., 5 to 10 membered, 5 to 9 membered, or 5 to 6 membered).

In embodiments, R⁸ is R⁴³-substituted or unsubstituted alkyl (e.g., C₁-C₈ alkyl, C₁-C₆ alkyl, or C₁-C₄ alkyl), R⁴³-substituted or unsubstituted heteroalkyl (e.g., 2 to 8 membered heteroalkyl, 2 to 6 membered heteroalkyl, or 2 to 4 membered heteroalkyl), R⁴³-substituted or unsubstituted cycloalkyl (e.g., C₃-C₈ cycloalkyl, C₃-C₆ cycloalkyl, or C₅-C₆ cycloalkyl), R⁴³-substituted or unsubstituted heterocycloalkyl (e.g., 3 to 8 membered heterocycloalkyl, 3 to 6 membered heterocycloalkyl, or 5 to 6 membered heterocycloalkyl), R⁴³-substituted or unsubstituted aryl (e.g., C₆-C₁₀ aryl, C₁₀ aryl, or phenyl), or R⁴³-substituted or unsubstituted heteroaryl (e.g., 5 to 10 membered heteroaryl, 5 to 9 membered heteroaryl, or 5 to 6 membered heteroaryl).

In embodiments, R⁸ is R⁴³-substituted or unsubstituted alkyl (e.g., C₁-C₈ alkyl, C₁-C₆ alkyl, or C₁-C₄ alkyl). In embodiments, R⁸ is R⁴³-substituted alkyl (e.g., C₁-C₈ alkyl, C₁-C₆ alkyl, or C₁-C₄ alkyl). In embodiments, R⁸ is an unsubstituted alkyl (e.g., C₁-C₈ alkyl, C₁-C₆ alkyl, or C₁-C₄ alkyl).

In embodiments, R⁸ is R⁴³-substituted or unsubstituted heteroalkyl (e.g., 2 to 8 membered heteroalkyl, 2 to 6 membered heteroalkyl, or 2 to 4 membered heteroalkyl). In embodiments, R⁸ is R⁴³-substituted heteroalkyl (e.g., 2 to 8 membered heteroalkyl, 2 to 6 membered heteroalkyl, or 2 to 4 membered heteroalkyl). In embodiments, R⁸ is an unsubstituted heteroalkyl (e.g., 2 to 8 membered heteroalkyl, 2 to 6 membered heteroalkyl, or 2 to 4 membered heteroalkyl).

In embodiments, R⁸ is R⁴³-substituted or unsubstituted cycloalkyl (e.g., C₃-C₈ cycloalkyl, C₃-C₆ cycloalkyl, or C₅-C₆ cycloalkyl). In embodiments, R⁸ is R⁴³-substituted cycloalkyl (e.g., C₃-C₈ cycloalkyl, C₃-C₆ cycloalkyl, or C₅-C₆ cycloalkyl). In embodiments, R⁸ is an unsubstituted cycloalkyl (e.g., C₃-C₈ cycloalkyl, C₃-C₆ cycloalkyl, or C₅-C₆ cycloalkyl).

In embodiments, R⁸ is R⁴³-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁸ is R⁴³-substituted heterocycloalkyl (e.g., 3 to 8 membered heterocycloalkyl, 3 to 6 membered heterocycloalkyl, or 5 to 6 membered heterocycloalkyl). In embodiments, R⁸ is an unsubstituted heterocycloalkyl (e.g., 3 to 8 membered heterocycloalkyl, 3 to 6 membered heterocycloalkyl, or 5 to 6 membered heterocycloalkyl).

In embodiments, R⁸ is R⁴³-substituted or unsubstituted aryl (e.g., C₆-C₁₀ aryl, C₁₀ aryl, or phenyl). In embodiments, R⁸ is R⁴³-substituted aryl (e.g., C₆-C₁₀ aryl, C₁₀ aryl, or phenyl). In embodiments, R⁸ is an unsubstituted aryl (e.g., C₆-C₁₀ aryl, C₁₀ aryl, or phenyl).

In embodiments, R⁸ is R⁴³-substituted or unsubstituted heteroaryl (e.g., 5 to 10 membered heteroaryl, 5 to 9 membered heteroaryl, or 5 to 6 membered heteroaryl). In embodiments, R⁸ is R⁴³-substituted heteroaryl (e.g., 5 to 10 membered heteroaryl, 5 to 9 membered heteroaryl, or 5 to 6 membered heteroaryl). In embodiments, R⁸ is an unsubstituted heteroaryl (e.g., 5 to 10 membered heteroaryl, 5 to 9 membered heteroaryl, or 5 to 6 membered heteroaryl).

R⁴³ is independently oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R⁴⁴-substituted or unsubstituted alkyl (e.g., C₁-C₈ alkyl, C₁-C₆ alkyl, or C₁-C₄ alkyl), R⁴⁴-substituted or unsubstituted heteroalkyl (e.g., 2 to 8 membered heteroalkyl, 2 to 6 membered heteroalkyl, or 2 to 4 membered heteroalkyl), R⁴⁴-substituted or unsubstituted cycloalkyl (e.g., C₃-C₈ cycloalkyl, C₃-C₆ cycloalkyl, or C₅-C₆ cycloalkyl), R⁴⁴-substituted or unsubstituted heterocycloalkyl (e.g., 3 to 8 membered heterocycloalkyl, 3 to 6 membered heterocycloalkyl, or 5 to 6 membered heterocycloalkyl), R⁴⁴-substituted or unsubstituted aryl (e.g., C₆-C₁₀ aryl, C₁₀ aryl, or phenyl), or R⁴⁴-substituted or unsubstituted heteroaryl (e.g., 5 to 10 membered heteroaryl, 5 to 9 membered heteroaryl, or 5 to 6 membered heteroaryl).

In embodiments, R⁴³ is R⁴⁴-substituted or unsubstituted alkyl (e.g., C₁-C₈ alkyl, C₁-C₆ alkyl, or C₁-C₄ alkyl), R⁴⁴-substituted or unsubstituted heteroalkyl (e.g., 2 to 8 membered heteroalkyl, 2 to 6 membered heteroalkyl, or 2 to 4 membered heteroalkyl), R⁴⁴-substituted or unsubstituted cycloalkyl (e.g., C₃-C₈ cycloalkyl, C₃-C₆ cycloalkyl, or C₅-C₆ cycloalkyl), R⁴⁴-substituted or unsubstituted heterocycloalkyl (e.g., 3 to 8 membered heterocycloalkyl, 3 to 6 membered heterocycloalkyl, or 5 to 6 membered heterocycloalkyl), R⁴⁴-substituted or unsubstituted aryl (e.g., C₆-C₁₀ aryl, C₁₀ aryl, or phenyl), or R⁴⁴-substituted or unsubstituted heteroaryl (e.g., 5 to 10 membered heteroaryl, 5 to 9 membered heteroaryl, or 5 to 6 membered heteroaryl).

In embodiments, R⁴³ is R⁴⁴-substituted or unsubstituted alkyl (e.g., C₁-C₈ alkyl, C₁-C₆ alkyl, or C₁-C₄ alkyl). In embodiments, R⁴³ is R⁴⁴-substituted alkyl (e.g., C₁-C₈ alkyl, C₁-C₆ alkyl, or C₁-C₄ alkyl). In embodiments, R⁴³ is an unsubstituted alkyl (e.g., C₁-C₈ alkyl, C₁-C₆ alkyl, or C₁-C₄ alkyl).

In embodiments, R⁴³ is R⁴⁴-substituted or unsubstituted heteroalkyl (e.g., 2 to 8 membered heteroalkyl, 2 to 6 membered heteroalkyl, or 2 to 4 membered heteroalkyl). In embodiments, R⁴³ is R⁴⁴-substituted heteroalkyl (e.g., 2 to 8 membered heteroalkyl, 2 to 6 membered heteroalkyl, or 2 to 4 membered heteroalkyl). In embodiments, R⁴³ is an unsubstituted heteroalkyl (e.g., 2 to 8 membered heteroalkyl, 2 to 6 membered heteroalkyl, or 2 to 4 membered heteroalkyl).

In embodiments, R⁴³ is R⁴⁴-substituted or unsubstituted cycloalkyl (e.g., C₃-C₈ cycloalkyl, C₃-C₆ cycloalkyl, or C₅-C₆ cycloalkyl). In embodiments, R⁴³ is R⁴⁴-substituted cycloalkyl (e.g., C₃-C₈ cycloalkyl, C₃-C₆ cycloalkyl, or C₅-C₆ cycloalkyl). In embodiments, R⁴³ is an unsubstituted cycloalkyl (e.g., C₃-C₈ cycloalkyl, C₃-C₆ cycloalkyl, or C₅-C₆ cycloalkyl).

In embodiments, R⁴³ is R⁴⁴-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁴³ is R⁴⁴-substituted heterocycloalkyl (e.g., 3 to 8 membered heterocycloalkyl, 3 to 6 membered heterocycloalkyl, or 5 to 6 membered heterocycloalkyl). In embodiments, R⁴³ is an unsubstituted heterocycloalkyl (e.g., 3 to 8 membered heterocycloalkyl, 3 to 6 membered heterocycloalkyl, or 5 to 6 membered heterocycloalkyl).

In embodiments, R⁴³ is R⁴⁴-substituted or unsubstituted aryl (e.g., C₆-C₁₀ aryl, C₁₀ aryl, or phenyl). In embodiments, R⁴³ is R⁴⁴-substituted aryl (e.g., C₆-C₁₀ aryl, C₁₀ aryl, or phenyl). In embodiments, R⁴³ is an unsubstituted aryl (e.g., C₆-C₁₀ aryl, C₁₀ aryl, or phenyl).

In embodiments, R⁴³ is R⁴⁴-substituted or unsubstituted heteroaryl (e.g., 5 to 10 membered heteroaryl, 5 to 9 membered heteroaryl, or 5 to 6 membered heteroaryl). In embodiments, R⁴³ is R⁴⁴-substituted heteroaryl (e.g., 5 to 10 membered heteroaryl, 5 to 9 membered heteroaryl, or 5 to 6 membered heteroaryl). In embodiments, R⁴³ is an unsubstituted heteroaryl (e.g., 5 to 10 membered heteroaryl, 5 to 9 membered heteroaryl, or 5 to 6 membered heteroaryl).

R⁴⁴ is independently oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R⁴⁵-substituted or unsubstituted alkyl (e.g., C₁-C₈ alkyl, C₁-C₆ alkyl, or C₁-C₄ alkyl), R⁴⁵-substituted or unsubstituted heteroalkyl (e.g., 2 to 8 membered heteroalkyl, 2 to 6 membered heteroalkyl, or 2 to 4 membered heteroalkyl), R⁴⁵-substituted or unsubstituted cycloalkyl (e.g., C₃-C₈ cycloalkyl, C₃-C₆ cycloalkyl, or C₅-C₆ cycloalkyl), R⁴⁵-substituted or unsubstituted heterocycloalkyl (e.g., 3 to 8 membered heterocycloalkyl, 3 to 6 membered heterocycloalkyl, or 5 to 6 membered heterocycloalkyl), R⁴⁵-substituted or unsubstituted aryl (e.g., C₆-C₁₀ aryl, C₁₀ aryl, or phenyl), or R⁴⁵-substituted or unsubstituted heteroaryl (e.g., 5 to 10 membered heteroaryl, 5 to 9 membered heteroaryl, or 5 to 6 membered heteroaryl).

In embodiments, R⁴⁴ is R⁴⁵-substituted or unsubstituted alkyl (e.g., C₁-C₈ alkyl, C₁-C₆ alkyl, or C₁-C₄ alkyl), R⁴⁵-substituted or unsubstituted heteroalkyl (e.g., 2 to 8 membered heteroalkyl, 2 to 6 membered heteroalkyl, or 2 to 4 membered heteroalkyl), R⁴⁵-substituted or unsubstituted cycloalkyl (e.g., C₃-C₈ cycloalkyl, C₃-C₆ cycloalkyl, or C₅-C₆ cycloalkyl), R⁴⁵-substituted or unsubstituted heterocycloalkyl (e.g., 3 to 8 membered heterocycloalkyl, 3 to 6 membered heterocycloalkyl, or 5 to 6 membered heterocycloalkyl), R⁴⁵-substituted or unsubstituted aryl (e.g., C₆-C₁₀ aryl, C₁₀ aryl, or phenyl), or R⁴⁵-substituted or unsubstituted heteroaryl (e.g., 5 to 10 membered heteroaryl, 5 to 9 membered heteroaryl, or 5 to 6 membered heteroaryl).

In embodiments, R⁴⁴ is R⁴⁵-substituted or unsubstituted alkyl (e.g., C₁-C₈ alkyl, C₁-C₆ alkyl, or C₁-C₄ alkyl). In embodiments, R⁴⁴ is R⁴⁵-substituted alkyl (e.g., C₁-C₈ alkyl, C₁-C₆ alkyl, or C₁-C₄ alkyl). In embodiments, R⁴⁴ is an unsubstituted alkyl (e.g., C₁-C₈ alkyl, C₁-C₆ alkyl, or C₁-C₄ alkyl).

In embodiments, R⁴⁴ is R⁴⁵-substituted or unsubstituted heteroalkyl (e.g., 2 to 8 membered heteroalkyl, 2 to 6 membered heteroalkyl, or 2 to 4 membered heteroalkyl). In embodiments, R⁴⁴ is R⁴⁵-substituted heteroalkyl (e.g., 2 to 8 membered heteroalkyl, 2 to 6 membered heteroalkyl, or 2 to 4 membered heteroalkyl). In embodiments, R⁴⁴ is an unsubstituted heteroalkyl (e.g., 2 to 8 membered heteroalkyl, 2 to 6 membered heteroalkyl, or 2 to 4 membered heteroalkyl).

In embodiments, R⁴⁴ is R⁴⁵-substituted or unsubstituted cycloalkyl (e.g., C₃-C₈ cycloalkyl, C₃-C₆ cycloalkyl, or C₅-C₆ cycloalkyl). In embodiments, R⁴⁴ is R⁴⁵-substituted cycloalkyl (e.g., C₃-C₈ cycloalkyl, C₃-C₆ cycloalkyl, or C₅-C₆ cycloalkyl). In embodiments, R⁴⁴ is an unsubstituted cycloalkyl (e.g., C₃-C₈ cycloalkyl, C₃-C₆ cycloalkyl, or C₅-C₆ cycloalkyl).

In embodiments, R⁴⁴ is R⁴⁵-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁴⁴ is R⁴⁵-substituted heterocycloalkyl (e.g., 3 to 8 membered heterocycloalkyl, 3 to 6 membered heterocycloalkyl, or 5 to 6 membered heterocycloalkyl). In embodiments, R⁴⁴ is an unsubstituted heterocycloalkyl (e.g., 3 to 8 membered heterocycloalkyl, 3 to 6 membered heterocycloalkyl, or 5 to 6 membered heterocycloalkyl).

In embodiments, R⁴⁴ is R⁴⁵-substituted or unsubstituted aryl (e.g., C₆-C₁₀ aryl, C₁₀ aryl, or phenyl). In embodiments, R⁴⁴ is R⁴⁵-substituted aryl (e.g., C₆-C₁₀ aryl, C₁₀ aryl, or phenyl). In embodiments, R⁴⁴ is an unsubstituted aryl (e.g., C₆-C₁₀ aryl, C₁₀ aryl, or phenyl).

In embodiments, R⁴⁴ is R⁴⁵-substituted or unsubstituted heteroaryl (e.g., 5 to 10 membered heteroaryl, 5 to 9 membered heteroaryl, or 5 to 6 membered heteroaryl). In embodiments, R⁴⁴ is R⁴⁵-substituted heteroaryl (e.g., 5 to 10 membered heteroaryl, 5 to 9 membered heteroaryl, or 5 to 6 membered heteroaryl). In embodiments, R⁴⁴ is an unsubstituted heteroaryl (e.g., 5 to 10 membered heteroaryl, 5 to 9 membered heteroaryl, or 5 to 6 membered heteroaryl).

R^{8.3}, R^{8.4}, and R^{8.5} are each hydrogen or R⁸ at a fixed position on the attached ring. R^{8.3}, R^{8.4}, and R^{8.5} may be any substituent of R⁸ described herein, including in any aspect, embodiment, example, figure, or claim. In embodiments, R^{8.4} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —CCl₃, —COOH, —CH₂COOH, —CONH₂, —OH, —SH, substituted or unsubstituted alkyl, or substituted or unsubstituted heteroalkyl. In embodiments, R^{8.4} is a protecting group. In embodiments, R^{8.4} is TMS. In embodiments, R^{8.3} and R^{8.5} are independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, or substituted or unsubstituted heteroalkyl.

In embodiments, R^{8.3}, R^{8.4}, R^{8.5} are each independently R⁴³-substituted or unsubstituted alkyl (e.g., C₁-C₈ alkyl, C₁-C₆ alkyl, or C₁-C₄ alkyl), R⁴³-substituted or unsubstituted heteroalkyl (e.g., 2 to 8 membered heteroalkyl, 2 to 6 membered heteroalkyl, or 2 to 4 membered heteroalkyl), R⁴³-substituted or unsubstituted cycloalkyl (e.g., C₃-C₈ cycloalkyl, C₃-C₆ cycloalkyl, or C₅-C₆ cycloalkyl), R⁴³-substituted or unsubstituted heterocycloalkyl (e.g., 3 to 8 membered heterocycloalkyl, 3 to 6 membered heterocycloalkyl, or 5 to 6 membered heterocycloalkyl), R⁴³-substituted or unsubstituted aryl (e.g., C₆-C₁₀ aryl, C₁₀ aryl, or phenyl), or R⁴³-substituted or unsubstituted heteroaryl (e.g., 5 to 10 membered heteroaryl, 5 to 9 membered heteroaryl, or 5 to 6 membered heteroaryl).

In embodiments, R^{8.3}, R^{8.4}, R^{8.5} are each independently R⁴³-substituted or unsubstituted alkyl (e.g., C₁-C₈ alkyl, C₁-C₆ alkyl, or C₁-C₄ alkyl). In embodiments, R^{8.3}, R^{8.4}, R^{8.5} are each independently R⁴³-substituted alkyl (e.g., C₁-C₈ alkyl, C₁-C₆ alkyl, or C₁-C₄ alkyl). In embodiments, R^{8.3}, R^{8.4}, R^{8.5} are each independently an unsubstituted alkyl (e.g., C₁-C₈ alkyl, C₁-C₆ alkyl, or C₁-C₄ alkyl).

In embodiments, R^{8.3}, R^{8.4}, R^{8.5} are each independently R⁴³-substituted or unsubstituted heteroalkyl (e.g., 2 to 8 membered heteroalkyl, 2 to 6 membered heteroalkyl, or 2 to 4 membered heteroalkyl). In embodiments, R^{8.3}, R^{8.4}, R^{8.5} are each independently R⁴³-substituted heteroalkyl (e.g., 2 to 8 membered heteroalkyl, 2 to 6 membered heteroalkyl, or 2 to 4 membered heteroalkyl). In embodiments, R^{8.3}, R^{8.4}, R^{8.5} are each independently an unsubstituted heteroalkyl (e.g., 2 to 8 membered heteroalkyl, 2 to 6 membered heteroalkyl, or 2 to 4 membered heteroalkyl).

In embodiments, R^{8.3}, R^{8.4}, R^{8.5} are each independently R⁴³-substituted or unsubstituted cycloalkyl (e.g., C₃-C₈ cycloalkyl, C₃-C₆ cycloalkyl, or C₅-C₆ cycloalkyl). In embodiments, R^{8.3}, R^{8.4}, R^{8.5} are each independently R⁴³-substituted cycloalkyl (e.g., C₃-C₈ cycloalkyl, C₃-C₆ cycloalkyl, or C₅-C₆ cycloalkyl). In embodiments, R^{8.3}, R^{8.4}, R^{8.5} are each independently an unsubstituted cycloalkyl (e.g., C₃-C₈ cycloalkyl, C₃-C₆ cycloalkyl, or C₅-C₆ cycloalkyl).

In embodiments, R^{8.3}, R^{8.4}, R^{8.5} are each independently R⁴³-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{8.3}, R^{8.4}, R^{8.5} are each independently R⁴³-substituted heterocycloalkyl (e.g., 3 to 8 membered heterocycloalkyl, 3 to 6 membered heterocycloalkyl, or 5 to 6 membered heterocycloalkyl). In embodiments, R^{8.3}, R^{8.4}, R^{8.5} each independently an unsubstituted heterocycloalkyl (e.g., 3 to 8 membered heterocycloalkyl, 3 to 6 membered heterocycloalkyl, or 5 to 6 membered heterocycloalkyl).

In embodiments, R^{8.3}, R^{8.4}, R^{8.5} are each independently R⁴³-substituted or unsubstituted aryl (e.g., C₆-C₁₀ aryl, C₁₀ aryl, or phenyl). In embodiments, R^{8.3}, R^{8.4}, R^{8.5} are each independently R⁴³-substituted aryl (e.g., C₆-C₁₀ aryl, C₁₀ aryl, or phenyl). In embodiments, R^{8.3}, R^{8.4}, R^{8.5} are each independently an unsubstituted aryl (e.g., C₆-C₁₀ aryl, C₁₀ aryl, or phenyl).

In embodiments, R^{8.3}, R^{8.4}, R^{8.5} are each independently R⁴³-substituted or unsubstituted heteroaryl (e.g., 5 to 10 membered heteroaryl, 5 to 9 membered heteroaryl, or 5 to 6 membered heteroaryl). In embodiments, R^{8.3}, R^{8.4}, R^{8.5} are each independently R⁴³-substituted heteroaryl (e.g., 5 to 10 membered heteroaryl, 5 to 9 membered heteroaryl, or 5 to 6 membered heteroaryl). In embodiments, R^{8.3}, R^{8.4}, R^{8.5} are each independently an unsubstituted heteroaryl (e.g., 5 to 10 membered heteroaryl, 5 to 9 membered heteroaryl, or 5 to 6 membered heteroaryl).

In embodiments, R⁹ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁹ is hydrogen, oxo, halogen, —CN, —OH, —NH₂, —COOH, —CONH₂, —NO₂, —SH, —SO₃H, or —N₃. In embodiments, R⁹ is an unsubstituted alkyl, or unsubstituted heteroalkyl. In embodiments, R⁹ is an unsubstituted alkyl (e.g., C₁-C₈, C₁-C₆, C₁-C₄, or C₁-C₂). In embodiments, R⁹ is an unsubstituted heteroalkyl (e.g., 2 to 8 membered, 2 to 6 membered, 4 to 6 membered, 2 to 3 membered, or 4 to 5 membered). In embodiments, R⁹ is hydrogen. In embodiments, R⁹ is oxo. In embodiments, R⁹ is halogen. In embodiments, R⁹ is —CN. In embodiments, R⁹ is —OH. In embodiments, R⁹ is —NH₂. In embodiments, R⁹ is —COOH. In embodiments, R⁹ is —CONH₂. In embodiments, R⁹ is —NO₂. In embodiments, R⁹ is —SH. In embodiments, R⁹ is —SO₃H. In embodiments, R⁹ is —N₃. In embodiments, R⁹ is oxo or halogen. In embodiments, R⁹ is oxo or —F. In embodiments, R⁹ is —F. In embodiments, R⁹ is halogen, oxo, —NH₂, unsubstituted alkyl, or unsubstituted heteroalkyl. In embodiments, R⁹ is —F, oxo, or —NH₂, or unsubstituted heteroalkyl. In embodiments, R⁹ is —NH₃ (e.g., a salt of NH₂).

In embodiments, R⁹ is hydrogen. In embodiments, R⁹ is oxo. In embodiments, R⁹ is halogen. In embodiments, R⁹ is —CCl₃. In embodiments, R⁹ is —CBr₃. In embodiments, R⁹ is —CF₃. In embodiments, R⁹ is —CI₃. In embodiments, R⁹ is CHCl₂. In embodiments, R⁹ is —CHBr₂. In embodiments, R⁹ is —CHF₂. In embodiments, R⁹ is —CHI₂. In embodiments, R⁹ is —CH₂C₁. In embodiments, R⁹ is —CH₂Br. In embodiments, R⁹ is —CH₂F. In embodiments, R⁹ is —CH₂I. In embodiments, R⁹ is —CN. In embodiments, R⁹ is —OH. In embodiments, R⁹ is —NH₂. In embodiments, R⁹ is —COOH. In embodiments, R⁹ is —CONH₂. In embodiments, R⁹ is —NO₂. In embodiments, R⁹ is —SH. In embodiments, R⁹ is —SO₃H. In embodiments, R⁹ is —SO₄H. In embodiments, R⁹ is —SO₂NH₂. In embodiments, R⁹ is —NHNH₂. In embodiments, R⁹ is —ONH₂. In embodiments, R⁹ is —NHC(O)NHNH₂. In embodiments, R⁹ is —NHC(O)NH₂. In embodiments, R⁹ is —NHSO₂H. In embodiments, R⁹ is —NHC(O)H, —NHC(O)OH. In embodiments, R⁹ is —NHOH. In embodiments, R⁹ is —OCCI₃. In embodiments, R⁹ is —OCF₃. In embodiments, R⁹ is —OCBr₃. In embodiments, R⁹ is —OCI₃. In embodiments, R⁹ is —OCHCl₂. In embodiments, R⁹ is —OCHBr₂. In embodiments, R⁹ is —OCHI₂. In embodiments, R⁹ is —OCHF₂. In embodiments, R⁹ is —OCH₂Cl. In embodiments, R⁹ is —OCH₂Br. In embodiments, R⁹ is —OCH₂I. In embodiments, R⁹ is —OCH₂F. In embodiments, R⁹ is —N₃.

In embodiments, R⁹ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R⁴⁶-substituted or unsubstituted alkyl (e.g., C₁-C₈ alkyl, C₁-C₆ alkyl, or C₁-C₄ alkyl), R⁴⁶-substituted or unsubstituted heteroalkyl (e.g., 2 to 8 membered heteroalkyl, 2 to 6 membered heteroalkyl, or 2 to 4 membered heteroalkyl), R⁴⁶-substituted or unsubstituted cycloalkyl (e.g., C₃-C₈ cycloalkyl, C₃-C₆ cycloalkyl, or C₅-C₆ cycloalkyl), R⁴⁶-substituted or unsubstituted heterocycloalkyl (e.g., 3 to 8 membered heterocycloalkyl, 3 to 6 membered heterocycloalkyl, or 5 to 6 membered heterocycloalkyl), R⁴⁶-substituted or unsubstituted aryl (e.g., C₆-C₁₀ aryl, C₁₀ aryl, or phenyl), or R⁴⁶-substituted or unsubstituted heteroaryl (e.g., 5 to 10 membered heteroaryl, 5 to 9 membered heteroaryl, or 5 to 6 membered heteroaryl).

In embodiments, R⁹ is R⁴⁶-substituted or unsubstituted alkyl (e.g., C₁-C₈ alkyl, C₁-C₆ alkyl, or C₁-C₄ alkyl), R⁴⁶-substituted or unsubstituted heteroalkyl (e.g., 2 to 8 membered heteroalkyl, 2 to 6 membered heteroalkyl, or 2 to 4 membered heteroalkyl), R⁴⁶-substituted or unsubstituted cycloalkyl (e.g., C₃-C₈ cycloalkyl, C₃-C₆ cycloalkyl, or C₅-C₆ cycloalkyl), R⁴⁶-substituted or unsubstituted heterocycloalkyl (e.g., 3 to 8 membered heterocycloalkyl, 3 to 6 membered heterocycloalkyl, or 5 to 6 membered heterocycloalkyl), R⁴⁶-substituted or unsubstituted aryl (e.g., C₆-C₁₀ aryl, C₁₀ aryl, or phenyl), or R⁴⁶-substituted or unsubstituted heteroaryl (e.g., 5 to 10 membered heteroaryl, 5 to 9 membered heteroaryl, or 5 to 6 membered heteroaryl).

In embodiments, R⁹ is R⁴⁶-substituted or unsubstituted alkyl (e.g., C₁-C₈ alkyl, C₁-C₆ alkyl, or C₁-C₄ alkyl). In embodiments, R⁹ is R⁴⁶-substituted alkyl (e.g., C₁-C₈ alkyl, C₁-C₆ alkyl, or C₁-C₄ alkyl). In embodiments, R⁹ is an unsubstituted alkyl (e.g., C₁-C₈ alkyl, C₁-C₆ alkyl, or C₁-C₄ alkyl).

In embodiments, R⁹ is R⁴⁶-substituted or unsubstituted heteroalkyl (e.g., 2 to 8 membered heteroalkyl, 2 to 6 membered heteroalkyl, or 2 to 4 membered heteroalkyl). In embodiments, R⁹ is R⁴⁶-substituted heteroalkyl (e.g., 2 to 8 membered heteroalkyl, 2 to 6 membered heteroalkyl, or 2 to 4 membered heteroalkyl). In embodiments, R⁹ is an unsubstituted heteroalkyl (e.g., 2 to 8 membered heteroalkyl, 2 to 6 membered heteroalkyl, or 2 to 4 membered heteroalkyl).

In embodiments, R⁹ is R⁴⁶-substituted or unsubstituted cycloalkyl (e.g., C₃-C₈ cycloalkyl, C₃-C₆ cycloalkyl, or C₅-C₆ cycloalkyl). In embodiments, R⁹ is R⁴⁶-substituted cycloalkyl (e.g., C₃-C₈ cycloalkyl, C₃-C₆ cycloalkyl, or C₅-C₆ cycloalkyl). In embodiments, R⁹ is an unsubstituted cycloalkyl (e.g., C₃-C₈ cycloalkyl, C₃-C₆ cycloalkyl, or C₅-C₆ cycloalkyl).

In embodiments, R⁹ is R⁴⁶-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R⁹ is R⁴⁶-substituted heterocycloalkyl (e.g., 3 to 8 membered heterocycloalkyl, 3 to 6 membered heterocycloalkyl, or 5 to 6 membered heterocycloalkyl). In embodiments, R⁹ is an unsubstituted heterocycloalkyl (e.g., 3 to 8 membered heterocycloalkyl, 3 to 6 membered heterocycloalkyl, or 5 to 6 membered heterocycloalkyl).

In embodiments, R⁹ is R⁴⁶-substituted or unsubstituted aryl (e.g., C₆-C₁₀ aryl, C₁₀ aryl, or phenyl). In embodiments, R⁹ is R⁴⁶-substituted aryl (e.g., C₆-C₁₀ aryl, C₁₀ aryl, or phenyl). In embodiments, R⁹ is an unsubstituted aryl (e.g., C₆-C₁₀ aryl, C₁₀ aryl, or phenyl).

In embodiments, R⁹ is R⁴⁶-substituted or unsubstituted heteroaryl (e.g., 5 to 10 membered heteroaryl, 5 to 9 membered heteroaryl, or 5 to 6 membered heteroaryl). In embodiments, R⁹ is R⁴⁶-substituted heteroaryl (e.g., 5 to 10 membered heteroaryl, 5 to 9 membered heteroaryl, or 5 to 6 membered heteroaryl). In embodiments, R⁹ is an unsubstituted heteroaryl (e.g., 5 to 10 membered heteroaryl, 5 to 9 membered heteroaryl, or 5 to 6 membered heteroaryl).

In embodiments, R¹⁰ is hydrogen or unsubstituted methyl. In embodiments, R¹⁰ is hydrogen. In embodiments, R¹⁰ is an unsubstituted C₁-C₃ alkyl. In embodiments, R¹⁰ is an unsubstituted C₂ alkyl. In embodiments, R¹⁰ is an unsubstituted C₃ alkyl. In embodiments, R¹⁰ is unsubstituted methyl. In embodiments, R¹⁰ and R¹² are independently hydrogen, substituted or unsubstituted C₁-C₃ alkyl, or substituted or unsubstituted 2 to 3 membered heteroalkyl.

In embodiments, R¹⁰ is substituted or unsubstituted C₁-C₃ alkyl, or substituted or unsubstituted 2 to 3 membered heteroalkyl. In embodiments, R¹⁰ is substituted (e.g., substituted with a substituent group, a size-limited substituent group, or lower substituent group) or unsubstituted alkyl. In embodiments, R¹⁰ is substituted (e.g., substituted with a substituent group, a size-limited substituent group, or lower substituent group) alkyl. In embodiments, R¹⁰ is unsubstituted alkyl. In embodiments, R¹⁰ is substituted or unsubstituted alkyl (e.g., C₁-C₈, C₁-C₆, C₁-C₄, or C₁-C₂). In embodiments, R¹⁰ is substituted alkyl (e.g., C₁-C₈, C₁-C₆, C₁-C₄, or C₁-C₂). In embodiments, R¹⁰ is unsubstituted alkyl (e.g., C₁-C₈, C₁-C₆, C₁-C₄, or C₁-C₂).

In embodiments, R¹⁰ is substituted (e.g., substituted with a substituent group, a size-limited substituent group, or lower substituent group) or unsubstituted heteroalkyl. In embodiments, R¹⁰ is substituted (e.g., substituted with a substituent group, a size-limited substituent group, or lower substituent group) heteroalkyl. In embodiments, R¹⁰ is unsubstituted heteroalkyl. In embodiments, R¹⁰ is substituted or unsubstituted heteroalkyl (e.g., 2 to 8 membered, 2 to 6 membered, 4 to 6 membered, 2 to 3 membered, or 4 to 5 membered). In embodiments, R¹⁰ is substituted heteroalkyl (e.g., 2 to 8 membered, 2 to 6 membered, 4 to 6 membered, 2 to 3 membered, or 4 to 5 membered). In embodiments, R¹⁰ is an unsubstituted heteroalkyl (e.g., 2 to 8 membered, 2 to 6 membered, 4 to 6 membered, 2 to 3 membered, or 4 to 5 membered).

In embodiments, R¹⁰ hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R⁴⁷-substituted or unsubstituted alkyl (e.g., C₁-C₈ alkyl, C₁-C₆ alkyl, or C₁-C₄ alkyl), R⁴⁷-substituted or unsubstituted heteroalkyl (e.g., 2 to 8 membered heteroalkyl, 2 to 6 membered heteroalkyl, or 2 to 4 membered heteroalkyl), R⁴⁷-substituted or unsubstituted cycloalkyl (e.g., C₃-C₈ cycloalkyl, C₃-C₆ cycloalkyl, or C₅-C₆ cycloalkyl), R⁴⁷-substituted or unsubstituted heterocycloalkyl (e.g., 3 to 8 membered heterocycloalkyl, 3 to 6 membered heterocycloalkyl, or 5 to 6 membered heterocycloalkyl), R⁴⁷-substituted or unsubstituted aryl (e.g., C₆-C₁₀ aryl, C₁₀ aryl, or phenyl), or R⁴⁷-substituted or unsubstituted heteroaryl (e.g., 5 to 10 membered heteroaryl, 5 to 9 membered heteroaryl, or 5 to 6 membered heteroaryl).

In embodiments, R¹⁰ is R⁴⁷-substituted or unsubstituted alkyl (e.g., C₁-C₈ alkyl, C₁-C₆ alkyl, or C₁-C₄ alkyl), R⁴⁷-substituted or unsubstituted heteroalkyl (e.g., 2 to 8 membered heteroalkyl, 2 to 6 membered heteroalkyl, or 2 to 4 membered heteroalkyl), R⁴⁷-substituted or unsubstituted cycloalkyl (e.g., C₃-C₈ cycloalkyl, C₃-C₆ cycloalkyl, or C₅-C₆ cycloalkyl), R⁴⁷-substituted or unsubstituted heterocycloalkyl (e.g., 3 to 8 membered heterocycloalkyl, 3 to 6 membered heterocycloalkyl, or 5 to 6 membered heterocycloalkyl), R⁴⁷-substituted or unsubstituted aryl (e.g., C₆-C₁₀ aryl, C₁₀ aryl, or phenyl), or R⁴⁷-substituted or unsubstituted heteroaryl (e.g., 5 to 10 membered heteroaryl, 5 to 9 membered heteroaryl, or 5 to 6 membered heteroaryl).

In embodiments, R¹⁰ is R⁴⁷-substituted or unsubstituted alkyl (e.g., C₁-C₈ alkyl, C₁-C₆ alkyl, or C₁-C₄ alkyl). In embodiments, R¹⁰ is R⁴⁷-substituted alkyl (e.g., C₁-C₈ alkyl, C₁-C₆ alkyl, or C₁-C₄ alkyl). In embodiments, R¹⁰ is an unsubstituted alkyl (e.g., C₁-C₈ alkyl, C₁-C₆ alkyl, or C₁-C₄ alkyl).

In embodiments, R¹⁰ is R⁴⁷-substituted or unsubstituted heteroalkyl (e.g., 2 to 8 membered heteroalkyl, 2 to 6 membered heteroalkyl, or 2 to 4 membered heteroalkyl). In embodiments, R¹⁰ is R⁴⁷-substituted heteroalkyl (e.g., 2 to 8 membered heteroalkyl, 2 to 6 membered heteroalkyl, or 2 to 4 membered heteroalkyl). In embodiments, R¹⁰ is an unsubstituted heteroalkyl (e.g., 2 to 8 membered heteroalkyl, 2 to 6 membered heteroalkyl, or 2 to 4 membered heteroalkyl).

In embodiments, R¹⁰ is R⁴⁷-substituted or unsubstituted cycloalkyl (e.g., C₃-C₈ cycloalkyl, C₃-C₆ cycloalkyl, or C₅-C₆ cycloalkyl). In embodiments, R¹⁰ is R⁴⁷-substituted cycloalkyl (e.g., C₃-C₈ cycloalkyl, C₃-C₆ cycloalkyl, or C₅-C₆ cycloalkyl). In embodiments, R¹⁰ is an unsubstituted cycloalkyl (e.g., C₃-C₈ cycloalkyl, C₃-C₆ cycloalkyl, or C₅-C₆ cycloalkyl).

In embodiments, R¹⁰ is R⁴⁷-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R¹⁰ is R⁴⁷-substituted heterocycloalkyl (e.g., 3 to 8 membered heterocycloalkyl, 3 to 6 membered heterocycloalkyl, or 5 to 6 membered heterocycloalkyl). In embodiments, R¹⁰ is an unsubstituted heterocycloalkyl (e.g., 3 to 8 membered heterocycloalkyl, 3 to 6 membered heterocycloalkyl, or 5 to 6 membered heterocycloalkyl).

In embodiments, R¹⁰ is R⁴⁷-substituted or unsubstituted aryl (e.g., C₆-C₁₀ aryl, C₁₀ aryl, or phenyl). In embodiments, R¹⁰ is R⁴⁷-substituted aryl (e.g., C₆-C₁₀ aryl, C₁₀ aryl, or phenyl). In embodiments, R¹⁰ is an unsubstituted aryl (e.g., C₆-C₁₀ aryl, C₁₀ aryl, or phenyl).

In embodiments, R¹⁰ is R⁴⁷-substituted or unsubstituted heteroaryl (e.g., 5 to 10 membered heteroaryl, 5 to 9 membered heteroaryl, or 5 to 6 membered heteroaryl). In embodiments, R¹⁰ is R⁴⁷-substituted heteroaryl (e.g., 5 to 10 membered heteroaryl, 5 to 9 membered heteroaryl, or 5 to 6 membered heteroaryl). In embodiments, R¹⁰ is an unsubstituted heteroaryl (e.g., 5 to 10 membered heteroaryl, 5 to 9 membered heteroaryl, or 5 to 6 membered heteroaryl).

In embodiments, R¹¹ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, or —N₃. In embodiments, R¹¹ is hydrogen. In embodiments, R¹¹ is oxo. In embodiments, R¹¹ is halogen. In embodiments, R¹¹ is —CCl₃. In embodiments, R¹¹ is —CBr₃. In embodiments, R¹¹ is —CF₃. In embodiments, R¹¹ is —CI₃. In embodiments, R¹¹ is CHCl₂. In embodiments, R¹¹ is —CHBr₂. In embodiments, R¹¹ is —CHF₂. In embodiments, R¹¹ is —CHI₂. In embodiments, R¹¹ is —CH₂C₁. In embodiments, R¹¹ is —CH₂Br. In embodiments, R¹¹ is —CH₂F. In embodiments, R¹¹ is —CH₂I. In embodiments, R¹¹ is —CN. In embodiments, R¹¹ is —OH. In embodiments, R¹¹ is —NH₂. In embodiments, R¹¹ is —COOH. In embodiments, R¹¹ is —CONH₂. In embodiments, R¹¹ is —NO₂. In embodiments, R¹¹ is —SH. In embodiments, R¹¹ is —SO₃H. In embodiments, R¹¹ is —SO₄H. In embodiments, R¹¹ is —SO₂NH₂. In embodiments, R¹¹ is —NHNH₂. In embodiments, R¹¹ is —ONH₂. In embodiments, R¹¹ is —NHC(O)NHNH₂. In embodiments, R¹¹ is —NHC(O)NH₂. In embodiments, R¹¹ is —NHSO₂H. In embodiments, R¹¹ is —NHC(O)H. In embodiments, R¹¹ is —NHC(O)OH. In embodiments, R¹¹ is —NHOH. In embodiments, R¹¹ is —OCCl₃. In embodiments, R¹¹ is —OCF₃. In embodiments, R¹¹ is —OCBr₃. In embodiments, R¹¹ is —OCI₃. In embodiments, R¹¹ is —OCHCl₂. In embodiments, R¹¹ is —OCHBr₂. In embodiments, R¹¹ is —OCHI₂. In embodiments, R¹¹ is —OCHF₂. In embodiments, R¹¹ is —OCH₂C₁. In embodiments, R¹¹ is —OCH₂Br. In embodiments, R¹¹ is —OCH₂I. In embodiments, R¹¹ is —OCH₂F. In embodiments, R¹¹ is —N₃. In embodiments, R¹¹ is —OH, —NH₂, or —SH.

In embodiments, R¹¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹¹ is substituted (e.g., substituted with a substituent group, a size-limited substituent group, or lower substituent group) or unsubstituted alkyl. In embodiments, R¹¹ is substituted (e.g., substituted with a substituent group, a size-limited substituent group, or lower substituent group) alkyl. In embodiments, R¹¹ is unsubstituted alkyl. In embodiments, R¹¹ is substituted or unsubstituted alkyl (e.g., C₁-C₈, C₁-C₆, C₁-C₄, or C₁-C₂). In embodiments, R¹¹ is substituted alkyl (e.g., C₁-C₈, C₁-C₆, C₁-C₄, or C₁-C₂). In embodiments, R¹¹ is unsubstituted alkyl (e.g., C₁-C₈, C₁-C₆, C₁-C₄, or C₁-C₂).

In embodiments, R¹¹ is substituted (e.g., substituted with a substituent group, a size-limited substituent group, or lower substituent group) or unsubstituted heteroalkyl. In embodiments, R¹¹ is substituted (e.g., substituted with a substituent group, a size-limited substituent group, or lower substituent group) heteroalkyl. In embodiments, R¹¹ is unsubstituted heteroalkyl. In embodiments, R¹¹ is substituted or unsubstituted heteroalkyl (e.g., 2 to 8 membered, 2 to 6 membered, 4 to 6 membered, 2 to 3 membered, or 4 to 5 membered). In embodiments, R¹¹ is substituted heteroalkyl (e.g., 2 to 8 membered, 2 to 6 membered, 4 to 6 membered, 2 to 3 membered, or 4 to 5 membered). In embodiments, R¹¹ is an unsubstituted heteroalkyl (e.g., 2 to 8 membered, 2 to 6 membered, 4 to 6 membered, 2 to 3 membered, or 4 to 5 membered).

In embodiments, R¹¹ is substituted (e.g., substituted with a substituent group, a size-limited substituent group, or lower substituent group) or unsubstituted cycloalkyl. In embodiments, R¹¹ is substituted (e.g., substituted with a substituent group, a size-limited substituent group, or lower substituent group) cycloalkyl. In embodiments, R¹¹ is an unsubstituted cycloalkyl. In embodiments, R¹¹ is substituted or unsubstituted cycloalkyl (e.g., C₃-C₈, C₃-C₆, C₄-C₆, or C₅-C₆). In embodiments, R¹¹ is substituted cycloalkyl (e.g., C₃-C₈, C₃-C₆, C₄-C₆, or C₅-C₆). In embodiments, R¹¹ is unsubstituted cycloalkyl (e.g., C₃-C₈, C₃-C₆, C₄-C₆, or C₅-C₆).

In embodiments, R¹¹ is substituted (e.g., substituted with a substituent group, a size-limited substituent group, or lower substituent group) or unsubstituted heterocycloalkyl. In embodiments, R¹¹ is substituted (e.g., substituted with a substituent group, a size-limited substituent group, or lower substituent group) heterocycloalkyl. In embodiments, R¹¹ is an unsubstituted heterocycloalkyl. In embodiments, R¹¹ is substituted or unsubstituted heterocycloalkyl (e.g., 3 to 8 membered, 3 to 6 membered, 4 to 6 membered, 4 to 5 membered, or 5 to 6 membered). In embodiments, R¹¹ is substituted heterocycloalkyl (e.g., 3 to 8 membered, 3 to 6 membered, 4 to 6 membered, 4 to 5 membered, or 5 to 6 membered). In embodiments, R¹¹ an unsubstituted heterocycloalkyl (e.g., 3 to 8 membered, 3 to 6 membered, 4 to 6 membered, 4 to 5 membered, or 5 to 6 membered).

In embodiments, R¹¹ is substituted (e.g., substituted with a substituent group, a size-limited substituent group, or lower substituent group) or unsubstituted aryl. In embodiments, R¹¹ is substituted (e.g., substituted with a substituent group, a size-limited substituent group, or lower substituent group) aryl. In embodiments, R¹¹ is an unsubstituted aryl. In embodiments, R¹¹ is substituted or unsubstituted aryl (e.g., C₆-C₁₀ or phenyl). In embodiments, R¹¹ is substituted aryl (e.g., C₆-C₁₀ or phenyl). In embodiments, R¹¹ is an unsubstituted aryl (e.g., C₆-C₁₀ or phenyl).

In embodiments, R¹¹ is substituted (e.g., substituted with a substituent group, a size-limited substituent group, or lower substituent group) or unsubstituted heteroaryl. In embodiments, R¹¹ is substituted (e.g., substituted with a substituent group, a size-limited substituent group, or lower substituent group) heteroaryl. In embodiments, R¹¹ is an unsubstituted heteroaryl. In embodiments, R¹¹ is substituted or unsubstituted heteroaryl (e.g., 5 to 10 membered, 5 to 9 membered, or 5 to 6 membered). In embodiments, R¹¹ is substituted heteroaryl (e.g., 5 to 10 membered, 5 to 9 membered, or 5 to 6 membered). In embodiments, R¹¹ is an unsubstituted heteroaryl (e.g., 5 to 10 membered, 5 to 9 membered, or 5 to 6 membered).

In embodiments, R¹¹ is oxo, halogen, —OH, or —NH₂. In embodiments, R¹¹ is —F.

In embodiments, R¹¹ hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCH F₂, —OCH₂Cl, —OCH₂Br, —OCH₂I, —OCH₂F, —N₃, R⁴⁸-substituted or unsubstituted alkyl (e.g., C₁-C₈ alkyl, C₁-C₆ alkyl, or C₁-C₄ alkyl), R⁴⁸-substituted or unsubstituted heteroalkyl (e.g., 2 to 8 membered heteroalkyl, 2 to 6 membered heteroalkyl, or 2 to 4 membered heteroalkyl), R⁴⁸-substituted or unsubstituted cycloalkyl (e.g., C₃-C₈ cycloalkyl, C₃-C₆ cycloalkyl, or C₅-C₆ cycloalkyl), R⁴⁸-substituted or unsubstituted heterocycloalkyl (e.g., 3 to 8 membered heterocycloalkyl, 3 to 6 membered heterocycloalkyl, or 5 to 6 membered heterocycloalkyl), R⁴⁸-substituted or unsubstituted aryl (e.g., C₆-C₁₀ aryl, C₁₀ aryl, or phenyl), or R⁴⁸-substituted or unsubstituted heteroaryl (e.g., 5 to 10 membered heteroaryl, 5 to 9 membered heteroaryl, or 5 to 6 membered heteroaryl).

In embodiments, R¹¹ is R⁴⁸-substituted or unsubstituted alkyl (e.g., C₁-C₈ alkyl, C₁-C₆ alkyl, or C₁-C₄ alkyl), R⁴⁸-substituted or unsubstituted heteroalkyl (e.g., 2 to 8 membered heteroalkyl, 2 to 6 membered heteroalkyl, or 2 to 4 membered heteroalkyl), R⁴⁸-substituted or unsubstituted cycloalkyl (e.g., C₃-C₈ cycloalkyl, C₃-C₆ cycloalkyl, or C₅-C₆ cycloalkyl), R⁴⁸-substituted or unsubstituted heterocycloalkyl (e.g., 3 to 8 membered heterocycloalkyl, 3 to 6 membered heterocycloalkyl, or 5 to 6 membered heterocycloalkyl), R⁴⁸-substituted or unsubstituted aryl (e.g., C₆-C₁₀ aryl, C₁₀ aryl, or phenyl), or R⁴⁸-substituted or unsubstituted heteroaryl (e.g., 5 to 10 membered heteroaryl, 5 to 9 membered heteroaryl, or 5 to 6 membered heteroaryl).

In embodiments, R¹¹ is R⁴⁸-substituted or unsubstituted alkyl (e.g., C₁-C₈ alkyl, C₁-C₆ alkyl, or C₁-C₄ alkyl). In embodiments, R¹¹ is R⁴⁸-substituted alkyl (e.g., C₁-C₈ alkyl, C₁-C₆ alkyl, or C₁-C₄ alkyl). In embodiments, R¹¹ is an unsubstituted alkyl (e.g., C₁-C₈ alkyl, C₁-C₆ alkyl, or C₁-C₄ alkyl).

In embodiments, R¹¹ is R⁴⁸-substituted or unsubstituted heteroalkyl (e.g., 2 to 8 membered heteroalkyl, 2 to 6 membered heteroalkyl, or 2 to 4 membered heteroalkyl). In embodiments, R¹¹ is R⁴⁸-substituted heteroalkyl (e.g., 2 to 8 membered heteroalkyl, 2 to 6 membered heteroalkyl, or 2 to 4 membered heteroalkyl). In embodiments, R¹¹ is an unsubstituted heteroalkyl (e.g., 2 to 8 membered heteroalkyl, 2 to 6 membered heteroalkyl, or 2 to 4 membered heteroalkyl).

In embodiments, R¹¹ is R⁴⁸-substituted or unsubstituted cycloalkyl (e.g., C₃-C₈ cycloalkyl, C₃-C₆ cycloalkyl, or C₅-C₆ cycloalkyl). In embodiments, R¹¹ is R⁴⁸-substituted cycloalkyl (e.g., C₃-C₈ cycloalkyl, C₃-C₆ cycloalkyl, or C₅-C₆ cycloalkyl). In embodiments, R¹¹ is an unsubstituted cycloalkyl (e.g., C₃-C₈ cycloalkyl, C₃-C₆ cycloalkyl, or C₅-C₆ cycloalkyl).

In embodiments, R¹¹ is R⁴⁸-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R¹¹ is R⁴⁸-substituted heterocycloalkyl (e.g., 3 to 8 membered heterocycloalkyl, 3 to 6 membered heterocycloalkyl, or 5 to 6 membered heterocycloalkyl). In embodiments, R¹¹ is an unsubstituted heterocycloalkyl (e.g., 3 to 8 membered heterocycloalkyl, 3 to 6 membered heterocycloalkyl, or 5 to 6 membered heterocycloalkyl).

In embodiments, R¹¹ is R⁴⁸-substituted or unsubstituted aryl (e.g., C₆-C₁₀ aryl, C₁₀ aryl, or phenyl). In embodiments, R¹¹ is R⁴⁸-substituted aryl (e.g., C₆-C₁₀ aryl, C₁₀ aryl, or phenyl). In embodiments, R¹¹ is an unsubstituted aryl (e.g., C₆-C₁₀ aryl, C₁₀ aryl, or phenyl).

In embodiments, R¹¹ is R⁴⁸-substituted or unsubstituted heteroaryl (e.g., 5 to 10 membered heteroaryl, 5 to 9 membered heteroaryl, or 5 to 6 membered heteroaryl). In embodiments, R¹¹ is R⁴⁸-substituted heteroaryl (e.g., 5 to 10 membered heteroaryl, 5 to 9 membered heteroaryl, or 5 to 6 membered heteroaryl). In embodiments, R¹¹ is an unsubstituted heteroaryl (e.g., 5 to 10 membered heteroaryl, 5 to 9 membered heteroaryl, or 5 to 6 membered heteroaryl).

In embodiments, R¹² is hydrogen or unsubstituted methyl. In embodiments, R¹² is hydrogen. In embodiments, R¹² is an unsubstituted C₁-C₃ alkyl. In embodiments, R¹² is an unsubstituted C₂ alkyl. In embodiments, R¹² is an unsubstituted C₃ alkyl. In embodiments, R¹² is unsubstituted methyl. In embodiments, R¹² is hydrogen or an unsubstituted C₁-C₃ alkyl.

In embodiments, R¹² is substituted or unsubstituted C₁-C₃ alkyl, or substituted or unsubstituted 2 to 3 membered heteroalkyl. In embodiments, R¹² is substituted (e.g., substituted with a substituent group, a size-limited substituent group, or lower substituent group) or unsubstituted alkyl. In embodiments, R¹² is substituted (e.g., substituted with a substituent group, a size-limited substituent group, or lower substituent group) alkyl. In embodiments, R¹² is unsubstituted alkyl. In embodiments, R¹² is substituted or unsubstituted alkyl (e.g., C₁-C₈, C₁-C₆, C₁-C₄, or C₁-C₂). In embodiments, R¹² is substituted alkyl (e.g., C₁-C₈, C₁-C₆, C₁-C₄, or C₁-C₂). In embodiments, R¹² is unsubstituted alkyl (e.g., C₁-C₈, C₁-C₆, C₁-C₄, or C₁-C₂).

In embodiments, R¹² is substituted (e.g., substituted with a substituent group, a size-limited substituent group, or lower substituent group) or unsubstituted heteroalkyl. In embodiments, R¹² is substituted (e.g., substituted with a substituent group, a size-limited substituent group, or lower substituent group) heteroalkyl. In embodiments, R¹² is unsubstituted heteroalkyl. In embodiments, R¹² is substituted or unsubstituted heteroalkyl (e.g., 2 to 8 membered, 2 to 6 membered, 4 to 6 membered, 2 to 3 membered, or 4 to 5 membered). In embodiments, R¹² is substituted heteroalkyl (e.g., 2 to 8 membered, 2 to 6 membered, 4 to 6 membered, 2 to 3 membered, or 4 to 5 membered). In embodiments, R¹² is an unsubstituted heteroalkyl (e.g., 2 to 8 membered, 2 to 6 membered, 4 to 6 membered, 2 to 3 membered, or 4 to 5 membered).

In embodiments, R¹² hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, R⁴⁹-substituted or unsubstituted alkyl (e.g., C₁-C₈ alkyl, C₁-C₆ alkyl, or C₁-C₄ alkyl), R⁴⁹-substituted or unsubstituted heteroalkyl (e.g., 2 to 8 membered heteroalkyl, 2 to 6 membered heteroalkyl, or 2 to 4 membered heteroalkyl), R⁴⁹-substituted or unsubstituted cycloalkyl (e.g., C₃-C₈ cycloalkyl, C₃-C₆ cycloalkyl, or C₅-C₆ cycloalkyl), R⁴⁹-substituted or unsubstituted heterocycloalkyl (e.g., 3 to 8 membered heterocycloalkyl, 3 to 6 membered heterocycloalkyl, or 5 to 6 membered heterocycloalkyl), R⁴⁹-substituted or unsubstituted aryl (e.g., C₆-C₁₀ aryl, C₁₀ aryl, or phenyl), or R⁴⁹-substituted or unsubstituted heteroaryl (e.g., 5 to 10 membered heteroaryl, 5 to 9 membered heteroaryl, or 5 to 6 membered heteroaryl).

In embodiments, R¹² is R⁴⁹-substituted or unsubstituted alkyl (e.g., C₁-C₈ alkyl, C₁-C₆ alkyl, or C₁-C₄ alkyl), R⁴⁹-substituted or unsubstituted heteroalkyl (e.g., 2 to 8 membered heteroalkyl, 2 to 6 membered heteroalkyl, or 2 to 4 membered heteroalkyl), R⁴⁹-substituted or unsubstituted cycloalkyl (e.g., C₃-C₈ cycloalkyl, C₃-C₆ cycloalkyl, or C₅-C₆ cycloalkyl), R⁴⁹-substituted or unsubstituted heterocycloalkyl (e.g., 3 to 8 membered heterocycloalkyl, 3 to 6 membered heterocycloalkyl, or 5 to 6 membered heterocycloalkyl), R⁴⁹-substituted or unsubstituted aryl (e.g., C₆-C₁₀ aryl, C₁₀ aryl, or phenyl), or R⁴⁹-substituted or unsubstituted heteroaryl (e.g., 5 to 10 membered heteroaryl, 5 to 9 membered heteroaryl, or 5 to 6 membered heteroaryl).

In embodiments, R¹² is R⁴⁹-substituted or unsubstituted alkyl (e.g., C₁-C₈ alkyl, C₁-C₆ alkyl, or C₁-C₄ alkyl). In embodiments, R¹² is R⁴⁹-substituted alkyl (e.g., C₁-C₈ alkyl, C₁-C₆ alkyl, or C₁-C₄ alkyl). In embodiments, R¹² is an unsubstituted alkyl (e.g., C₁-C₈ alkyl, C₁-C₆ alkyl, or C₁-C₄ alkyl).

In embodiments, R¹² is R⁴⁹-substituted or unsubstituted heteroalkyl (e.g., 2 to 8 membered heteroalkyl, 2 to 6 membered heteroalkyl, or 2 to 4 membered heteroalkyl). In embodiments, R¹² is R⁴⁹-substituted heteroalkyl (e.g., 2 to 8 membered heteroalkyl, 2 to 6 membered heteroalkyl, or 2 to 4 membered heteroalkyl). In embodiments, R¹² is an unsubstituted heteroalkyl (e.g., 2 to 8 membered heteroalkyl, 2 to 6 membered heteroalkyl, or 2 to 4 membered heteroalkyl).

In embodiments, R¹² is R⁴⁹-substituted or unsubstituted cycloalkyl (e.g., C₃-C₈ cycloalkyl, C₃-C₆ cycloalkyl, or C₅-C₆ cycloalkyl). In embodiments, R¹² is R⁴⁹-substituted cycloalkyl (e.g., C₃-C₈ cycloalkyl, C₃-C₆ cycloalkyl, or C₅-C₆ cycloalkyl). In embodiments, R¹² is an unsubstituted cycloalkyl (e.g., C₃-C₈ cycloalkyl, C₃-C₆ cycloalkyl, or C₅-C₆ cycloalkyl).

In embodiments, R¹² is R⁴⁹-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R¹² is R⁴⁹-substituted heterocycloalkyl (e.g., 3 to 8 membered heterocycloalkyl, 3 to 6 membered heterocycloalkyl, or 5 to 6 membered heterocycloalkyl). In embodiments, R¹² is an unsubstituted heterocycloalkyl (e.g., 3 to 8 membered heterocycloalkyl, 3 to 6 membered heterocycloalkyl, or 5 to 6 membered heterocycloalkyl).

In embodiments, R¹² is R⁴⁹-substituted or unsubstituted aryl (e.g., C₆-C₁₀ aryl, C₁₀ aryl, or phenyl). In embodiments, R¹² is R⁴⁹-substituted aryl (e.g., C₆-C₁₀ aryl, C₁₀ aryl, or phenyl). In embodiments, R¹² is an unsubstituted aryl (e.g., C₆-C₁₀ aryl, C₁₀ aryl, or phenyl).

In embodiments, R¹² is R⁴⁹-substituted or unsubstituted heteroaryl (e.g., 5 to 10 membered heteroaryl, 5 to 9 membered heteroaryl, or 5 to 6 membered heteroaryl). In embodiments, R¹² is R⁴⁹-substituted heteroaryl (e.g., 5 to 10 membered heteroaryl, 5 to 9 membered heteroaryl, or 5 to 6 membered heteroaryl). In embodiments, R¹² is an unsubstituted heteroaryl (e.g., 5 to 10 membered heteroaryl, 5 to 9 membered heteroaryl, or 5 to 6 membered heteroaryl).

In embodiments, R¹³ is an organotin heteroalkyl, having the formula:

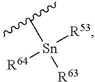

wherein R⁵³, R⁶³, and R⁶⁴ are independently unsubstituted alkyl (e.g., C₁-C₈ alkyl, C₁-C₆ alkyl, or C₁-C₄ alkyl) or unsubstituted aryl (e.g., C₆-C₁₀ aryl, C₁₀ aryl, or phenyl). In embodiments, R¹³ is

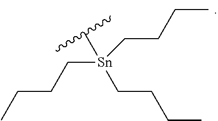 [SMILES: [*][Sn]([CH2]CCC)([CH2]CCC)[CH2]CCC]

In embodiments, R¹³ is a halogen. In embodiments, R¹³ is —Br.

In embodiments, R¹³ is R⁵³-substituted or unsubstituted alkyl (e.g., C₁-C₈ alkyl, C₁-C₆ alkyl, or C₁-C₄ alkyl), R⁵³-substituted or unsubstituted heteroalkyl (e.g., 2 to 8 membered heteroalkyl, 2 to 6 membered heteroalkyl, or 2 to 4 membered heteroalkyl), R⁵³-substituted or unsubstituted cycloalkyl (e.g., C₃-C₈ cycloalkyl, C₃-C₆ cycloalkyl, or C₅-C₆ cycloalkyl), R⁵³-substituted or unsubstituted heterocycloalkyl (e.g., 3 to 8 membered heterocycloalkyl, 3 to 6 membered heterocycloalkyl, or 5 to 6 membered heterocycloalkyl), R⁵³-substituted or unsubstituted aryl (e.g., C₆-C₁₀ aryl, C₁₀ aryl, or phenyl), or R⁵³-substituted or unsubstituted heteroaryl (e.g., 5 to 10 membered heteroaryl, 5 to 9 membered heteroaryl, or 5 to 6 membered heteroaryl).

In embodiments, R¹³ is R⁵³-substituted or unsubstituted alkyl (e.g., C₁-C₈ alkyl, C₁-C₆ alkyl, or C₁-C₄ alkyl). In embodiments, R¹³ is R⁵³-substituted alkyl (e.g., C₁-C₈ alkyl, C₁-C₆ alkyl, or C₁-C₄ alkyl). In embodiments, R¹³ is an unsubstituted alkyl (e.g., C₁-C₈ alkyl, C₁-C₆ alkyl, or C₁-C₄ alkyl).

In embodiments, R¹³ is R⁵³-substituted or unsubstituted heteroalkyl (e.g., 2 to 8 membered heteroalkyl, 2 to 6 membered heteroalkyl, or 2 to 4 membered heteroalkyl). In embodiments, R¹³ is R⁵³-substituted heteroalkyl (e.g., 2 to 8 membered heteroalkyl, 2 to 6 membered heteroalkyl, or 2 to 4 membered heteroalkyl). In embodiments, R¹³ is an unsubstituted heteroalkyl (e.g., 2 to 8 membered heteroalkyl, 2 to 6 membered heteroalkyl, or 2 to 4 membered heteroalkyl).

In embodiments, R¹³ is R⁵³-substituted or unsubstituted cycloalkyl (e.g., C₃-C₈ cycloalkyl, C₃-C₆ cycloalkyl, or C₅-C₆ cycloalkyl). In embodiments, R¹³ is R⁵³-substituted cycloalkyl (e.g., C₃-C₈ cycloalkyl, C₃-C₆ cycloalkyl, or C₅-C₆ cycloalkyl). In embodiments, R¹³ is an unsubstituted cycloalkyl (e.g., C₃-C₈ cycloalkyl, C₃-C₆ cycloalkyl, or C₅-C₆ cycloalkyl).

In embodiments, R¹³ is R⁵³-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R¹³ is R⁵³-substituted heterocycloalkyl (e.g., 3 to 8 membered heterocycloalkyl, 3 to 6 membered heterocycloalkyl, or 5 to 6 membered heterocycloalkyl). In embodiments, R¹³ is an unsubstituted heterocycloalkyl (e.g., 3 to 8 membered heterocycloalkyl, 3 to 6 membered heterocycloalkyl, or 5 to 6 membered heterocycloalkyl).

In embodiments, R¹³ is R⁵³-substituted or unsubstituted aryl (e.g., C₆-C₁₀ aryl, C₁₀ aryl, or phenyl). In embodiments, R¹³ is R⁵³-substituted aryl (e.g., C₆-C₁₀ aryl, C₁₀ aryl, or phenyl). In embodiments, R¹³ is an unsubstituted aryl (e.g., C₆-C₁₀ aryl, C₁₀ aryl, or phenyl).

In embodiments, R¹³ is R⁵³-substituted or unsubstituted heteroaryl (e.g., 5 to 10 membered heteroaryl, 5 to 9 membered heteroaryl, or 5 to 6 membered heteroaryl). In embodiments, R¹³ is R⁵³-substituted heteroaryl (e.g., 5 to 10 membered heteroaryl, 5 to 9 membered heteroaryl, or 5 to 6 membered heteroaryl). In embodiments, R¹³ is an unsubstituted heteroaryl (e.g., 5 to 10 membered heteroaryl, 5 to 9 membered heteroaryl, or 5 to 6 membered heteroaryl).

In embodiments, Ring A is a substituted (e.g., R⁸-substituted) or unsubstituted C₃-C₆ cycloalkylene, substituted (e.g., R⁸-substituted) or unsubstituted 3 to 6 membered heterocycloalkylene, substituted (e.g., R⁸-substituted) or unsubstituted phenylene, or substituted (e.g., R⁸-substituted) or unsubstituted 5 to 6 membered heteroarylene. In embodiments, Ring A is a substituted (e.g., R⁸-substituted) or unsubstituted 5 to 6 membered heteroarylene. In embodiments, Ring A is a substituted (e.g., R⁸-substituted) or unsubstituted oxazolylene.

In embodiments, Ring A is substituted (e.g., R⁸-substituted) or unsubstituted cycloalkylene. In embodiments, Ring A is substituted (e.g., R⁸-substituted) or unsubstituted heterocycloalkylene. In embodiments, Ring A is substituted (e.g., R⁸-substituted) or unsubstituted arylene. In embodiments, Ring A is substituted (e.g., R⁸-substituted) or unsubstituted heteroarylene. In embodiments, Ring A is substituted (e.g., R⁸-substituted) or unsubstituted (C₃-C₁₀) cycloalkylene, substituted (e.g., R⁸-substituted) or unsubstituted 3 to 10 membered heterocycloalkylene, substituted (e.g., R⁸-substituted) or unsubstituted (C₆-C₁₀) arylene, or substituted (e.g., R⁸-substituted) or unsubstituted 5 to 10 membered heteroarylene. In embodiments, Ring A is substituted (e.g., R⁸-substituted) or unsubstituted (C₃-C₁₀) cycloalkylene. In embodiments, Ring A is substituted (e.g., R⁸-substituted) or unsubstituted 3 to 10 membered heterocycloalkylene. In embodiments, Ring A is substituted (e.g., R⁸-substituted) or unsubstituted (C₆-C₁₀) arylene. In embodiments, Ring A is substituted (e.g., R⁸-substituted) or unsubstituted 5 to 10 membered heteroarylene. In embodiments, Ring A is substituted (e.g., R⁸-substituted) or unsubstituted (C₃-C₆) cycloalkylene. In embodiments, Ring A is substituted (e.g., R⁸-substituted) or unsubstituted 3 to 6 membered heterocycloalkylene. In embodiments, Ring A is substituted (e.g., R⁸-substituted) or unsubstituted phenylene. In embodiments, Ring A is substituted (e.g., R⁸-substituted) or unsubstituted naphthylene. In embodiments, Ring A is substituted (e.g., R⁸-substituted) or unsubstituted 5 to 9 membered heteroarylene. In embodiments, Ring A is substituted (e.g., R⁸-substituted) or unsubstituted 5 to 6 membered heteroarylene. In embodiments, Ring A is an unsubstituted 5 to 6 membered heteroarylene. In embodiments, Ring A is substituted (e.g., R⁸-substituted) or unsubstituted 5 membered heteroarylene. In embodiments, Ring A is a substituted (e.g., R⁸-substituted) 5 membered heteroarylene. In embodiments, Ring A is an unsubstituted 5 membered heteroarylene.

In embodiments, Ring A is R⁸-substituted or unsubstituted (C₃-C₁₀) cycloalkylene, R⁸-substituted or unsubstituted 5 to 10 membered heterocycloalkylene, R⁸-substituted or unsubstituted (C₆-C₁₀) arylene, or R⁸-substituted or unsubstituted 5 to 10 membered heteroarylene. In embodiments, Ring A is R⁸-substituted or unsubstituted (C₃-C₁₀) cycloalkylene or R⁸-substituted or unsubstituted 5 to 10 membered heterocycloalkylene. In embodiments, Ring A is R⁸-substituted or unsubstituted (C₃-C₁₀) cycloalkylene. In embodiments, Ring A is R⁸-substituted or unsubstituted 3 to 10 membered heterocycloalkylene. In embodiments, Ring A is R⁸-substituted or unsubstituted (C₆-C₁₀) arylene. In embodiments, Ring A is R⁸-substituted or unsubstituted 5 to 10 membered heteroarylene. In embodiments, Ring A is R⁸-substituted or unsubstituted (C₃-C₆) cycloalkylene. In embodiments, Ring A is R⁸-substituted or unsubstituted 3 to 6 membered heterocycloalkylene. In embodiments, Ring A is R⁸-substituted or unsubstituted phenylene. In embodiments, Ring A is R⁸-substituted or unsubstituted naphthylene. In embodiments, Ring A is R⁸-substituted or unsubstituted 5 to 9 membered heteroarylene. In embodiments, Ring A is R⁸-substituted or unsubstituted 5 to 6 membered heteroarylene.

In embodiments, Ring A is R⁸-substituted or unsubstituted thienylene. In embodiments, Ring A is R⁸-substituted or unsubstituted phenylene. In embodiments, Ring A is R⁸-substituted or unsubstituted benzothienylene. In embodiments, Ring A is R⁸-substituted or unsubstituted naphthylene. In embodiments, Ring A is R⁸-substituted or unsubstituted benzofuranylene. In embodiments, Ring A is R⁸-substituted or unsubstituted furanylene. In embodiments, Ring A is R⁸-substituted or unsubstituted pyrrolylene. In embodiments, Ring A is R⁸-substituted or unsubstituted oxazolylene. In embodiments, Ring A is R⁸-substituted or unsubstituted oxadiazolylene. In embodiments, Ring A is R⁸-substituted or unsubstituted triazolylene. In embodiments, Ring A is R⁸-substituted or unsubstituted thiazolylene.

In embodiments, Ring A is substituted (e.g., R⁸-substituted) cycloalkylene. In embodiments, Ring A is substituted (e.g., R⁸-substituted) heterocycloalkylene. In embodiments, Ring A is substituted (e.g., R⁸-substituted) arylene. In embodiments, Ring A is substituted (e.g., R⁸-substituted) heteroarylene. In embodiments, Ring A is substituted (e.g., R⁸-substituted) (C₃-C₁₀) cycloalkylene, substituted (e.g., R⁸-substituted) 3 to 10 membered heterocycloalkylene, substituted (e.g., R⁸-substituted) (C₆-C₁₀) arylene, or substituted (e.g., R⁸-substituted) 5 to 10 membered heteroarylene. In embodiments, Ring A is substituted (e.g., R⁸-substituted) (C₃-C₁₀) cycloalkylene. In embodiments, Ring A is substituted (e.g., R⁸-substituted) 3 to 10 membered heterocycloalkylene. In embodiments, Ring A is substituted (e.g., R⁸-substituted) (C₆-C₁₀) arylene. In embodiments, Ring A is substituted (e.g., R⁸-substituted) 5 to 10 membered heteroarylene. In embodiments, Ring A is substituted (e.g., R⁸-substituted) (C₃-C₆) cycloalkylene. In embodiments, Ring A is substituted (e.g., R⁸-substituted) 3 to 6 membered heterocycloalkylene. In embodiments, Ring A is substituted (e.g., R⁸-substituted) phenylene. In embodiments, Ring A is substituted (e.g., R⁸-substituted) naphthylene. In embodiments, Ring A is substituted (e.g., R⁸-substituted) 5 to 9 membered heteroarylene. In embodiments, Ring A is substituted (e.g., R⁸-substituted) 5 to 6 membered heteroarylene. In embodiments, Ring A is R⁸-substituted (C₃-C₁₀) cycloalkylene, R⁸-substituted 5 to 10 membered heterocycloalkylene, R⁸-substituted (C₆-C₁₀) arylene, or R⁸-substituted 5 to 10 membered heteroarylene. In embodiments, Ring A is R⁸-substituted (C₃-C₁₀) cycloalkylene or R⁸-substituted 5 to 10 membered heterocycloalkylene. In embodiments, Ring A is R⁸-substituted (C₃-C₁₀) cycloalkylene. In embodiments, Ring A is R⁸-substituted 3 to 10 membered heterocycloalkylene. In embodiments, Ring A is R⁸-substituted (C₆-C₁₀) arylene. In embodiments, Ring A is R⁸-substituted 5 to 10 membered heteroarylene. In embodiments, Ring A is R⁸-substituted (C₃-C₆) cycloalkylene. In embodiments, Ring A is R⁸-substituted 3 to 6 membered heterocycloalkylene. In embodiments, Ring A is R⁸-substituted phenylene. In embodiments, Ring A is R⁸-substituted naphthylene. In embodiments, Ring A is R⁸-substituted 5 to 9 membered heteroarylene. In embodiments, Ring A is R⁸-substituted 5 to 6 membered heteroarylene. In embodiments, Ring A is R⁸-substituted thienylene. In embodiments, Ring A is R⁸-substituted phenylene. In embodiments, Ring A is R⁸-substituted benzothienylene. In embodiments, Ring A is R⁸-substituted naphthylene. In embodiments, Ring A is R⁸-substituted benzofuranylene. In embodiments, Ring A is R⁸-substituted furanylene. In embodiments, Ring A is R⁸-substituted pyrrolylene. In embodiments, Ring A is R⁸-substituted oxazolylene. In embodiments, Ring A is R⁸-substituted oxadiazdylene. In embodiments, Ring A is R⁸-substituted triazolylene. In embodiments, Ring A is R⁸-substituted thiazolylene.

In embodiments, Ring A is unsubstituted cycloalkylene. In embodiments, Ring A is unsubstituted heterocycloalkylene. In embodiments, Ring A is unsubstituted arylene. In embodiments, Ring A is unsubstituted heteroarylene. In embodiments, Ring A is unsubstituted (C₃-C₁₀) cycloalkylene, unsubstituted 3 to 10 membered heterocycloalkylene, unsubstituted (C₆-C₁₀) arylene, or unsubstituted 5 to 10 membered heteroarylene. In embodiments, Ring A is unsubstituted (C₃-C₁₀) cycloalkylene. In embodiments, Ring A is unsubstituted 3 to 10 membered heterocycloalkylene. In embodiments, Ring A is unsubstituted (C₆-C₁₀) arylene. In embodiments, Ring A is unsubstituted 5 to 10 membered heteroarylene. In embodiments, Ring A is unsubstituted (C₃-C₁₀) cycloalkylene. In embodiments, Ring A is unsubstituted 3 to 6 membered heterocycloalkylene. In embodiments, Ring A is unsubstituted phenylene. In embodiments, Ring A is unsubstituted naphthylene. In embodiments, Ring A is unsubstituted 5 to 9 membered heteroarylene. In embodiments, Ring A is unsubstituted 5 to 6 membered heteroarylene. In embodiments, Ring A is unsubstituted (C₃-C₁₀) cycloalkylene, unsubstituted 5 to 10 membered heterocycloalkylene, unsubstituted (C₆-C₁₀) arylene, or unsubstituted 5 to 10 membered heteroarylene. In embodiments, Ring A is unsubstituted (C₃-C₁₀) cycloalkylene or unsubstituted 5 to 10 membered heterocycloalkylene. In embodiments, Ring A is unsubstituted (C₃-C₁₀) cycloalkylene. In embodiments, Ring A is unsubstituted 3 to 10 membered heterocycloalkylene. In embodiments, Ring A is unsubstituted (C₆-C₁₀) arylene. In embodiments, Ring A is unsubstituted 5 to 10 membered heteroarylene. In embodiments, Ring A is unsubstituted (C₃-C₆) cycloalkylene. In embodiments, Ring A is unsubstituted 3 to 6 membered heterocycloalkylene. In embodiments, Ring A is unsubstituted phenylene. In embodiments, Ring A is unsubstituted naphthylene. In embodiments, Ring A is unsubstituted 5 to 9 membered heteroarylene. In embodiments, Ring A is unsubstituted 5 to 6 membered heteroarylene. In embodiments, Ring A is unsubstituted thienylene. In embodiments, Ring A is unsubstituted phenylene. In embodiments, Ring A is unsubstituted benzothienylene. In embodiments, Ring A is unsubstituted naphthylene. In embodiments, Ring A is unsubstituted benzofuranylene. In embodiments, Ring A is unsubstituted furanylene. In embodiments, Ring A is unsubstituted pyrrolylene. In embodiments, Ring A is an unsubstituted oxazolylene. In embodiments, Ring A is unsubstituted phenylene. In embodiments, Ring A is an unsubstituted oxadiazolylene. In embodiments, Ring A is an unsubstituted triazolylene. In embodiments, Ring A is an unsubstituted thiazolylene.

In embodiments, Ring A is C₆-C₁₀ arylene or 5 to 10 membered heteroarylene. In embodiments, Ring A is C₆-C₁₀ arylene or 5 to 10 membered heteroarylene. In embodiments, Ring A is C₆-C₁₀ arylene. In embodiments, Ring A is phenylene. In embodiments, Ring A is naphthylene. In embodiments, Ring A is 5 to 10 membered heteroarylene. In embodiments, Ring A is 5 to 6 membered heteroarylene. In embodiments, Ring A is thienylene. In embodiments, Ring A is furanylene. In embodiments, Ring A is pyrrolylene. In embodiments, Ring A is imidazolylene. In embodiments, Ring A is pyrazolylene. In embodiments, Ring A is oxazolylene. In embodiments, Ring A is isoxazolylene. In embodiments, Ring A is thaizolylene. In embodiments, Ring A is pyridinylene. In embodiments, Ring A is pyridylene. In embodiments, Ring A is pyrazinylene. In embodiments, Ring A is pyrimidinylene. In embodiments, Ring A is pyridazinylene. In embodiments, Ring A is 1,2,3-triazinylene. In embodiments, Ring A is 1,2,4-triazinylene. In embodiments, Ring A is 1,3,5-triazinylene.

In embodiments, Ring A is substituted (e.g., R⁸-substituted) C₆-C₁₀ arylene or substituted (e.g., R⁸-substituted) 5 to 10 membered heteroarylene. In embodiments, Ring A is substituted (e.g., R⁸-substituted) C₆-C₁₀ arylene or substituted (e.g., R⁸-substituted) 5 to 10 membered heteroarylene. In embodiments, Ring A is substituted (e.g., R⁸-substituted) C₆-C₁₀ arylene. In embodiments, Ring A is substituted (e.g., R⁸-substituted) phenylene. In embodiments, Ring A is substituted (e.g., R⁸-substituted) naphthylene. In embodiments, Ring A is substituted (e.g., R⁸-substituted) 5 to 10 membered heteroarylene. In embodiments, Ring A is substituted (e.g., R⁸-substituted) 5 to 6 membered heteroarylene. In embodiments, Ring A is substituted (e.g., R⁸-substituted) thienylene. In embodiments, Ring A is substituted (e.g., R⁸-substituted) furanylene. In embodiments, Ring A is substituted (e.g., R⁸-substituted) pyrrolylene. In embodiments, Ring A is substituted (e.g., R⁸-substituted) imidazolylene. In embodiments, Ring A is substituted (e.g., R⁸-substituted) pyrazolylene. In embodiments, Ring A is substituted (e.g., R⁸-substituted) oxazolylene. In embodiments, Ring A is substituted (e.g., R⁸-substituted) isoxazolylene. In embodiments, Ring A is substituted (e.g., R⁸-substituted) thaizolylene. In embodiments, Ring A is substituted (e.g., R⁸-substituted) pyridinylene. In embodiments, Ring A is substituted (e.g., R⁸-substituted) pyridylene. In embodiments, Ring A is substituted (e.g., R⁸-substituted) pyrazinylene. In embodiments, Ring A is substituted (e.g., R⁸-substituted) pyrimidinylene. In embodiments, Ring A is substituted (e.g., R⁸-substituted) pyridazinylene. In embodiments, Ring A is substituted (e.g., R⁸-substituted) 1,2,3-triazinylene. In embodiments, Ring A is substituted (e.g., R⁸-substituted) 1,2,4-triazinylene. In embodiments, Ring A is substituted (e.g., R⁸-substituted) 1,3,5-triazinylene.

In embodiments, Ring A is an unsubstituted C₆-C₁₀ arylene or unsubstituted 5 to 10 membered heteroarylene. In embodiments, Ring A is an unsubstituted C₆-C₁₀ arylene or unsubstituted 5 to 10 membered heteroarylene. In embodiments, Ring A is an unsubstituted C₆-C₁₀ arylene. In embodiments, Ring A is an unsubstituted phenylene. In embodiments, Ring A is an unsubstituted naphthylene. In embodiments, Ring A is an unsubstituted 5 to 10 membered heteroarylene. In embodiments, Ring A is an unsubstituted 5 to 6 membered heteroarylene. In embodiments, Ring A is an unsubstituted thienylene. In embodiments, Ring A is an unsubstituted furanylene. In embodiments, Ring A is an unsubstituted pyrrolylene. In embodiments, Ring A is an unsubstituted imidazolylene. In embodiments, Ring A is an unsubstituted pyrazolylene. In embodiments, Ring A is an unsubstituted oxazolylene. In embodiments, Ring A is an unsubstituted isoxazolylene. In embodiments, Ring A is an unsubstituted thaizolylene. In embodiments, Ring A is an unsubstituted pyridinylene. In embodiments, Ring A is an unsubstituted pyridylene. In embodiments, Ring A is an unsubstituted pyrazinylene. In embodiments, Ring A is an unsubstituted pyrimidinylene. In embodiments, Ring A is an unsubstituted pyridazinylene. In embodiments, Ring A is an unsubstituted 1,2,3-triazinylene. In embodiments, Ring A is an unsubstituted 1,2,4-triazinylene. In embodiments, Ring A is an unsubstituted 1,3,5-triazinylene.

In embodiments, Ring A is a 4 to 8 membered heterocycloalkylene. In embodiments, Ring A is a 4 membered heterocycloalkylene. In embodiments, Ring A is a 5 membered heterocycloalkylene. In embodiments, Ring A is a 6 membered heterocycloalkylene. In embodiments, Ring A is a 7 membered heterocycloalkylene. In embodiments, Ring A is an 8 membered heterocycloalkylene. In embodiments, Ring A is a 4 to 6 membered heterocycloalkylene. In embodiments, Ring A is azetidinylene.

In embodiments, Ring A is a substituted (e.g., R⁸-substituted) or unsubstituted heterocycloalkylene. In embodiments, Ring A is a substituted (e.g., R⁸-substituted) heterocycloalkylene. In embodiments, Ring A is an unsubstituted heterocycloalkylene. In embodiments, Ring A is a substituted (e.g., R⁸-substituted) or unsubstituted 3 to 10 membered heterocycloalkylene. In embodiments, Ring A is a substituted (e.g., R⁸-substituted) 3 to 10 membered heterocycloalkylene. In embodiments, Ring A is an unsubstituted 3 to 10 membered heterocycloalkylene. In embodiments, Ring A is a R⁶-substituted or unsubstituted 5 to 10 membered heterocycloalkylene. In embodiments, Ring A is a R⁶-substituted 5 to 10 membered heterocycloalkylene. In embodiments, Ring A is an unsubstituted 5 to 10 membered heterocycloalkylene.

In embodiments, Ring A is a substituted (e.g., R⁸-substituted) or unsubstituted 3 membered heterocycloalkylene. In embodiments, Ring A is a substituted (e.g., R⁸-substituted) or unsubstituted 4 membered heterocycloalkylene. In embodiments, Ring A is a substituted (e.g., R⁸-substituted) or unsubstituted 5 membered heterocycloalkylene. In embodiments, Ring A is a substituted (e.g., R⁸-substituted) or unsubstituted 6 membered heterocycloalkylene. In embodiments, Ring A is a substituted (e.g., R⁸-substituted) 3 membered heterocycloalkylene. In embodiments, Ring A is a substituted (e.g., R⁸-substituted) 4 membered heterocycloalkylene. In embodiments, Ring A is a substituted (e.g., R⁸-substituted) 5 membered heterocycloalkylene. In embodiments, Ring A is a substituted (e.g., R⁸-substituted) 6 membered heterocycloalkylene. In embodiments, Ring A is an unsubstituted 3 membered heterocycloalkylene. In embodiments, Ring A is an unsubstituted 4 membered heterocycloalkylene. In embodiments, Ring A is an unsubstituted 5 membered heterocycloalkylene. In embodiments, Ring A is an unsubstituted 6 membered heterocycloalkylene.

In embodiments, Ring A is a substituted (e.g., R⁸-substituted) (i.e., R⁸-substituted) or unsubstituted aziridinylene, azirinylene, azetidinylene, dihydroazetylene, diazetidinylene, azetylene, pyrrolidinylene, pyrrolinylene, pyrrolylene, pyrazolidinylene, imidazolidinylene, pyrazolinylene, pyrazolylene, thiazolidinylene, thiazolylene, isothiazolylene, piperidinylene, piperazinylene, morpholinylene, oxazinylene, thiomorpholinylene, thiazinylene, decahydroquinolinylene, dihydroazepinylene, azepanylene, or azocanylene. In embodiments, Ring A is a substituted (e.g., R⁸-substituted) (i.e., R⁸-substituted) aziridinylene, azirinylene, azetidinylene, dihydroazetylene, diazetidinylene, azetylene, pyrrolidinylene, pyrrolinylene, pyrrolylene, pyrazolidinylene, imidazolidinylene, pyrazolinylene, pyrazolylene, thiazolidinylene, thiazolylene, isothiazolylene, piperidinylene, piperazinylene, morpholinylene, oxazinylene, thiomorpholinylene, thiazinylene, decahydroquinolinylene, dihydroazepinylene, azepanylene, or azocanylene. In embodiments, Ring A is an unsubstituted aziridinylene, azirinylene, azetidinylene, dihydroazetylene, diazetidinylene, azetylene, pyrrolidinylene, pyrrolinylene, pyrrolylene, pyrazolidinylene, imidazolidinylene, pyrazolinylene, pyrazolylene, thiazolidinylene, thiazolylene, isothiazolylene, piperidinylene, piperazinylene, morpholinylene, oxazinylene, thiomorpholinylene, thiazinylene, decahydroquinolinylene, dihydroazepinylene, azepanylene, or azocanylene.

In embodiments, Ring A is imidazolylene, pyrrolylene, pyrazolylene, triazolylene, tetrazolylene, furanylene, oxazolylene, isooxazolylene, oxadiazolylene, oxatriazolylene, thienylene, thiazolylene, isothiazolylene, pyridinylene, pyrazinylene, pyrimidinylene, pyridazinylene, or triazinylene.

In embodiments, Ring A is oxazolylene, thiazolylene, isooxazolylene, or oxadiazolylene.

In embodiments, Ring A is

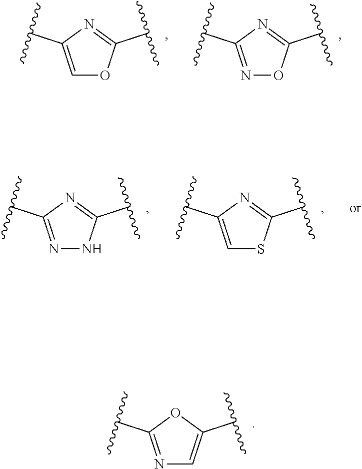 [SMILES: *c1coc(*)n1 ; *c1noc(*)n1 ; *c1n[nH]c(*)n1 ; *c1csc(*)n1 ; *c1cnc(*)o1]

Ring A may be substituted with one R⁸. Ring A may be substituted with two optionally different R⁸ substituents. Ring A may be substituted with three optionally different R⁸ substituents. Ring A may be substituted with four optionally different R⁸ substituents. Ring A may be substituted with five optionally different R⁸ substituents. Ring A may be substituted with six optionally different R⁸ substituents. Ring A may be substituted with seven optionally different R⁸ substituents. Ring A may be substituted with eight optionally different R⁸ substituents. Ring A may be substituted with nine optionally different R⁸ substituents. Ring A may be substituted with ten optionally different R⁸ substituents.

R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, and R⁶² are independently oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, and R⁶² are independently halogen. In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, and R⁶² are independently unsubstituted methyl.

In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, and R⁶² are independently unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, and R⁶² are independently unsubstituted alkyl (e.g., C₁-C₈ alkyl, C₁-C₆ alkyl, or C₁-C₄ alkyl). In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, and R⁶² are independently unsubstituted heteroalkyl (e.g., 2 to 8 membered heteroalkyl, 2 to 6 membered heteroalkyl, or 2 to 4 membered heteroalkyl). In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, and R⁶² are independently unsubstituted cycloalkyl (e.g., C₃-C₈ cycloalkyl, C₃-C₆ cycloalkyl, or C₅-C₆ cycloalkyl). In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, and R⁶² are independently unsubstituted heterocycloalkyl (e.g., 3 to 8 membered heterocycloalkyl, 3 to 6 membered heterocycloalkyl, or 5 to 6 membered heterocycloalkyl). In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, and R⁶² are independently unsubstituted aryl (e.g., C₆-C₁₀ aryl, C₁₀ aryl, or phenyl). In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, and R⁶² are independently unsubstituted heteroaryl (e.g., 5 to 10 membered heteroaryl, 5 to 9 membered heteroaryl, or 5 to 6 membered heteroaryl).

In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, and R⁶² are independently halogen. In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, and R⁶² are independently unsubstituted methyl.

In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, and R⁶² are independently unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, and R⁶² are independently unsubstituted alkyl (e.g., C₁-C₈ alkyl, C₁-C₆ alkyl, or C₁-C₄ alkyl). In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, and R⁶² are independently unsubstituted heteroalkyl (e.g., 2 to 8 membered heteroalkyl, 2 to 6 membered heteroalkyl, or 2 to 4 membered heteroalkyl). In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, and R⁶² are independently unsubstituted cycloalkyl (e.g., C₃-C₈ cycloalkyl, C₃-C₆ cycloalkyl, or C₅-C₆ cycloalkyl). In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, and R⁶² are independently unsubstituted heterocycloalkyl (e.g., 3 to 8 membered heterocycloalkyl, 3 to 6 membered heterocycloalkyl, or 5 to 6 membered heterocycloalkyl). In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, and R⁶² are independently unsubstituted aryl (e.g., C₆-C₁₀ aryl, C₁₀ aryl, or phenyl). In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, and R⁶² are independently unsubstituted heteroaryl (e.g., 5 to 10 membered heteroaryl, 5 to 9 membered heteroaryl, or 5 to 6 membered heteroaryl).

In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, or R⁶² are independently halogen. In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, or R⁶² are independently unsubstituted methyl.

In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, or R⁶² are independently unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, or R⁶² are independently unsubstituted alkyl (e.g., C₁-C₈ alkyl, C₁-C₆ alkyl, or C₁-C₄ alkyl). In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, or R⁶² are independently unsubstituted heteroalkyl (e.g., 2 to 8 membered heteroalkyl, 2 to 6 membered heteroalkyl, or 2 to 4 membered heteroalkyl). In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, or R⁶² are independently unsubstituted cycloalkyl (e.g., C₃-C₈ cycloalkyl, C₃-C₆ cycloalkyl, or C₅-C₆ cycloalkyl). In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, or R⁶² are independently unsubstituted heterocycloalkyl (e.g., 3 to 8 membered heterocycloalkyl, 3 to 6 membered heterocycloalkyl, or 5 to 6 membered heterocycloalkyl). In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, or R⁶² are independently unsubstituted aryl (e.g., C₆-C₁₀ aryl, C₁₀ aryl, or phenyl). In embodiments, R³⁷, R³⁸, R⁴¹, R⁴², R⁴⁵, R⁴⁶, R⁴⁷, R⁴⁸, R⁴⁹, R⁵³, R⁵⁴, R⁵⁵, R⁵⁶, R⁵⁷, R⁵⁸, R⁵⁹, R⁶⁰, R⁶¹, or R⁶² are independently unsubstituted heteroaryl (e.g., 5 to 10 membered heteroaryl, 5 to 9 membered heteroaryl, or 5 to 6 membered heteroaryl).

In embodiments, z8 is an integer from 0 to 5. In embodiments, z8 is an integer from 0 to 3. In embodiments, z8 is an integer from 0 to 2. In embodiments, z8 is an integer from 1 to 2. In embodiments, z8 is 0. In embodiments, z8 is 1. In embodiments, z8 is 2. In embodiments, z8 is 3. In embodiments, z8 is 4. In embodiments, z8 is 5. In embodiments, z8 is 6. In embodiments, z8 is 7. In embodiments, z8 is 8. In embodiments, z8 is 9. In embodiments, z8 is 10.

In embodiments, compound A has the formula:

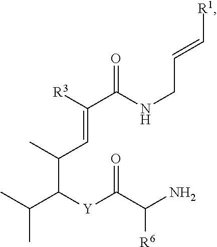

wherein R¹, R³, Y, and R⁶ are as described herein. In embodiments, the terminal amine (i.e., —NH₂) may lose a hydrogen to react and form a bond with R²⁴. In embodiments, the terminal amine (i.e., —NH₂) may lose a hydrogen to react and form a bond with the carbon atom to which R²⁴ is attached. In embodiments, compound A has the formula:

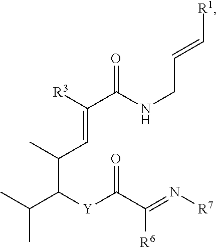

wherein R¹, R³, Y, R⁶, and R⁷ are as described herein.

In embodiments, compound A has the formula:

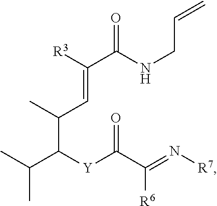

wherein R³, Y, R⁶, and R⁷ are as described herein.

In embodiments, compound A has the formula:

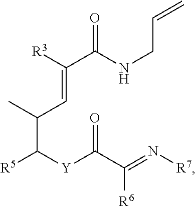

wherein R⁵, R³, Y, R⁶, and R⁷ are as described herein.

In embodiments, compound A has the formula:

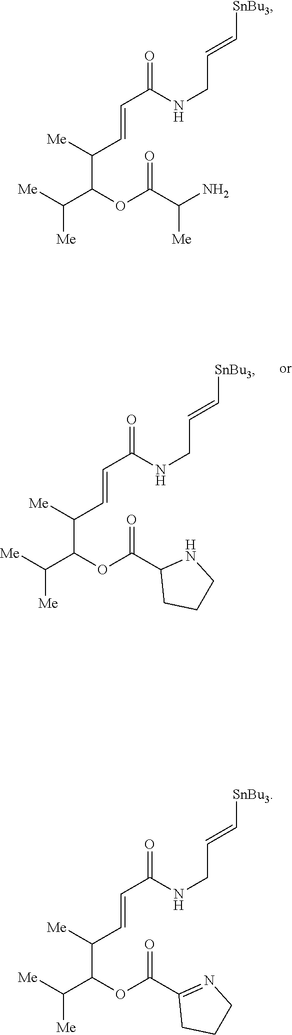

In embodiments, compound A2 has the formula:

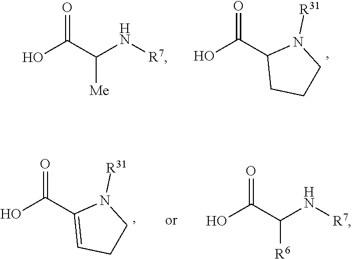

wherein R⁶ and R⁷ is as described herein.

R³¹ is a leaving group, protecting group, hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, compound A2 has the formula:

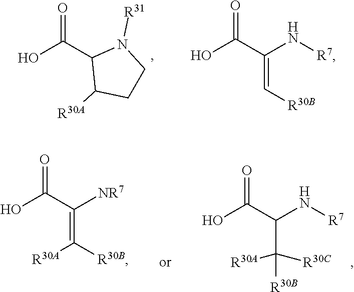

wherein R⁷ and R³¹ are as described herein.

R^{30A}, R^{30B}, and R^{30C} are independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl (e.g., C₁-C₈ alkyl, C₁-C₆ alkyl, or C₁-C₄ alkyl), substituted or unsubstituted heteroalkyl (e.g., 2 to 8 membered heteroalkyl, 2 to 6 membered heteroalkyl, or 2 to 4 membered heteroalkyl), substituted or unsubstituted cycloalkyl (e.g., C₃-C₈ cycloalkyl, C₃-C₆ cycloalkyl, or C₅-C₆ cycloalkyl), substituted or unsubstituted heterocycloalkyl (e.g., 3 to 8 membered heterocycloalkyl, 3 to 6 membered heterocycloalkyl, or 5 to 6 membered heterocycloalkyl), substituted or unsubstituted aryl (e.g., C₆-C₁₀ aryl, C₁₀ aryl, or phenyl), or substituted or unsubstituted heteroaryl (e.g., 5 to 10 membered heteroaryl, 5 to 9 membered heteroaryl, or 5 to 6 membered heteroaryl).

In embodiments, R³¹, R^{30A}, R^{30B}, and R^{30C} are independently substituted (e.g., substituted with a substituent group, a size-limited substituent group, or lower substituent group) or unsubstituted alkyl, substituted (e.g., substituted with a substituent group, a size-limited substituent group, or lower substituent group) or unsubstituted heteroalkyl, substituted (e.g., substituted with a substituent group, a size-limited substituent group, or lower substituent group) or unsubstituted cycloalkyl, substituted (e.g., substituted with a substituent group, a size-limited substituent group, or lower substituent group) or unsubstituted heterocycloalkyl, substituted (e.g., substituted with a substituent group, a size-limited substituent group, or lower substituent group) or unsubstituted aryl, or substituted (e.g., substituted with a substituent group, a size-limited substituent group, or lower substituent group) or unsubstituted heteroaryl. In embodiments, R³¹, R^{30A}, R^{30B}, and R^{30C} are hydrogen.

In embodiments, R³¹, R^{30A}, R^{30B}, and R^{30C} are unsubstituted alkyl, unsubstituted heteroalkyl, unsubstituted cycloalkyl, unsubstituted heterocycloalkyl, unsubstituted aryl, or unsubstituted heteroaryl.

In embodiments, R^{30A} is R⁵⁸-substituted or unsubstituted alkyl (e.g., C₁-C₈ alkyl, C₁-C₆ alkyl, or C₁-C₄ alkyl), R⁵⁸-substituted or unsubstituted heteroalkyl (e.g., 2 to 8 membered heteroalkyl, 2 to 6 membered heteroalkyl, or 2 to 4 membered heteroalkyl), R⁵⁸-substituted or unsubstituted cycloalkyl (e.g., C₃-C₈ cycloalkyl, C₃-C₆ cycloalkyl, or C₅-C₆ cycloalkyl), R⁵⁸-substituted or unsubstituted heterocycloalkyl (e.g., 3 to 8 membered heterocycloalkyl, 3 to 6 membered heterocycloalkyl, or 5 to 6 membered heterocycloalkyl), R⁵⁸-substituted or unsubstituted aryl (e.g., C₆-C₁₀ aryl, C₁₀ aryl, or phenyl), or R⁵⁸-substituted or unsubstituted heteroaryl (e.g., 5 to 10 membered heteroaryl, 5 to 9 membered heteroaryl, or 5 to 6 membered heteroaryl).

In embodiments, R^{30A} is R⁵⁸-substituted or unsubstituted alkyl (e.g., C₁-C₈ alkyl, C₁-C₆ alkyl, or C₁-C₄ alkyl). In embodiments, R^{30A} is R⁵⁸-substituted alkyl (e.g., C₁-C₈ alkyl, C₁-C₆ alkyl, or C₁-C₄ alkyl). In embodiments, R^{30A} is an unsubstituted alkyl (e.g., C₁-C₈ alkyl, C₁-C₆ alkyl, or C₁-C₄ alkyl).

In embodiments, R^{30A} is R⁵⁸-substituted or unsubstituted heteroalkyl (e.g., 2 to 8 membered heteroalkyl, 2 to 6 membered heteroalkyl, or 2 to 4 membered heteroalkyl). In embodiments, R^{30A} is R⁵⁸-substituted heteroalkyl (e.g., 2 to 8 membered heteroalkyl, 2 to 6 membered heteroalkyl, or 2 to 4 membered heteroalkyl). In embodiments, R^{30A} is an unsubstituted heteroalkyl (e.g., 2 to 8 membered heteroalkyl, 2 to 6 membered heteroalkyl, or 2 to 4 membered heteroalkyl).

In embodiments, R^{30A} is R⁵⁸-substituted or unsubstituted cycloalkyl (e.g., C₃-C₈ cycloalkyl, C₃-C₆ cycloalkyl, or C₅-C₆ cycloalkyl). In embodiments, R^{30A} is R⁵⁸-substituted cycloalkyl (e.g., C₃-C₈ cycloalkyl, C₃-C₆ cycloalkyl, or C₅-C₆ cycloalkyl). In embodiments, R^{30A} is an unsubstituted cycloalkyl (e.g., C₃-C₈ cycloalkyl, C₃-C₆ cycloalkyl, or C₅-C₆ cycloalkyl).

In embodiments, R^{30A} is R⁵⁸-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{30A} is R⁵⁸-substituted heterocycloalkyl (e.g., 3 to 8 membered heterocycloalkyl, 3 to 6 membered heterocycloalkyl, or 5 to 6 membered heterocycloalkyl). In embodiments, R^{30A} is an unsubstituted heterocycloalkyl (e.g., 3 to 8 membered heterocycloalkyl, 3 to 6 membered heterocycloalkyl, or 5 to 6 membered heterocycloalkyl).

In embodiments, R^{30A} is R⁵⁸-substituted or unsubstituted aryl (e.g., C₆-C₁₀ aryl, C₁₀ aryl, or phenyl). In embodiments, R^{30A} is R⁵⁸-substituted aryl (e.g., C₆-C₁₀ aryl, C₁₀ aryl, or phenyl). In embodiments, R^{30A} is an unsubstituted aryl (e.g., C₆-C₁₀ aryl, C₁₀ aryl, or phenyl).

In embodiments, R^{30A} is R⁵⁸-substituted or unsubstituted heteroaryl (e.g., 5 to 10 membered heteroaryl, 5 to 9 membered heteroaryl, or 5 to 6 membered heteroaryl). In embodiments, R^{30A} is R⁵⁸-substituted heteroaryl (e.g., 5 to 10 membered heteroaryl, 5 to 9 membered heteroaryl, or 5 to 6 membered heteroaryl). In embodiments, R^{30A} is an unsubstituted heteroaryl (e.g., 5 to 10 membered heteroaryl, 5 to 9 membered heteroaryl, or 5 to 6 membered heteroaryl).

In embodiments, R^{30B} is R⁵⁸-substituted or unsubstituted alkyl (e.g., C₁-C₈ alkyl, C₁-C₆ alkyl, or C₁-C₄ alkyl), R⁵⁸-substituted or unsubstituted heteroalkyl (e.g., 2 to 8 membered heteroalkyl, 2 to 6 membered heteroalkyl, or 2 to 4 membered heteroalkyl), R⁵⁸-substituted or unsubstituted cycloalkyl (e.g., C₃-C₈ cycloalkyl, C₃-C₆ cycloalkyl, or C₅-C₆ cycloalkyl), R⁵⁸-substituted or unsubstituted heterocycloalkyl (e.g., 3 to 8 membered heterocycloalkyl, 3 to 6 membered heterocycloalkyl, or 5 to 6 membered heterocycloalkyl), R⁵⁸-substituted or unsubstituted aryl (e.g., C₆-C₁₀ aryl, C₁₀ aryl, or phenyl), or R⁵⁸-substituted or unsubstituted heteroaryl (e.g., 5 to 10 membered heteroaryl, 5 to 9 membered heteroaryl, or 5 to 6 membered heteroaryl).

In embodiments, R^{30B} is R⁵⁸-substituted or unsubstituted alkyl (e.g., C₁-C₈ alkyl, C₁-C₆ alkyl, or C₁-C₄ alkyl). In embodiments, R^{30B} is R⁵⁸-substituted alkyl (e.g., C₁-C₈ alkyl, C₁-C₆ alkyl, or C₁-C₄ alkyl). In embodiments, R^{30B} is an unsubstituted alkyl (e.g., C₁-C₈ alkyl, C₁-C₆ alkyl, or C₁-C₄ alkyl).

In embodiments, R^{30B} is R⁵⁸-substituted or unsubstituted heteroalkyl (e.g., 2 to 8 membered heteroalkyl, 2 to 6 membered heteroalkyl, or 2 to 4 membered heteroalkyl). In embodiments, R^{30B} is R⁵⁸-substituted heteroalkyl (e.g., 2 to 8 membered heteroalkyl, 2 to 6 membered heteroalkyl, or 2 to 4 membered heteroalkyl). In embodiments, R^{30B} is an unsubstituted heteroalkyl (e.g., 2 to 8 membered heteroalkyl, 2 to 6 membered heteroalkyl, or 2 to 4 membered heteroalkyl).

In embodiments, R^{30B} is R⁵⁸-substituted or unsubstituted cycloalkyl (e.g., C₃-C₈ cycloalkyl, C₃-C₆ cycloalkyl, or C₅-C₆ cycloalkyl). In embodiments, R^{30B} is R⁵⁸-substituted cycloalkyl (e.g., C₃-C₈ cycloalkyl, C₃-C₆ cycloalkyl, or C₅-C₆ cycloalkyl). In embodiments, R^{30B} is an unsubstituted cycloalkyl (e.g., C₃-C₈ cycloalkyl, C₃-C₆ cycloalkyl, or C₅-C₆ cycloalkyl).

In embodiments, R^{30B} is R⁵⁸-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{30B} is R⁵⁸-substituted heterocycloalkyl (e.g., 3 to 8 membered heterocycloalkyl, 3 to 6 membered heterocycloalkyl, or 5 to 6 membered heterocycloalkyl). In embodiments, R^{30B} is an unsubstituted heterocycloalkyl (e.g., 3 to 8 membered heterocycloalkyl, 3 to 6 membered heterocycloalkyl, or 5 to 6 membered heterocycloalkyl).

In embodiments, R^{30B} is R⁵⁸-substituted or unsubstituted aryl (e.g., C₆-C₁₀ aryl, C₁₀ aryl, or phenyl). In embodiments, R^{30B} is R⁵⁸-substituted aryl (e.g., C₆-C₁₀ aryl, C₁₀ aryl, or phenyl). In embodiments, R^{30B} is an unsubstituted aryl (e.g., C₆-C₁₀ aryl, C₁₀ aryl, or phenyl).

In embodiments, R^{30B} is R⁵⁸-substituted or unsubstituted heteroaryl (e.g., 5 to 10 membered heteroaryl, 5 to 9 membered heteroaryl, or 5 to 6 membered heteroaryl). In embodiments, R^{30B} is R⁵⁸-substituted heteroaryl (e.g., 5 to 10 membered heteroaryl, 5 to 9 membered heteroaryl, or 5 to 6 membered heteroaryl). In embodiments, R^{30B} is an unsubstituted heteroaryl (e.g., 5 to 10 membered heteroaryl, 5 to 9 membered heteroaryl, or 5 to 6 membered heteroaryl).

In embodiments, R^{30C} is R⁵⁸-substituted or unsubstituted alkyl (e.g., C₁-C₈ alkyl, C₁-C₆ alkyl, or C₁-C₄ alkyl), R⁵⁸-substituted or unsubstituted heteroalkyl (e.g., 2 to 8 membered heteroalkyl, 2 to 6 membered heteroalkyl, or 2 to 4 membered heteroalkyl), R⁵⁸-substituted or unsubstituted cycloalkyl (e.g., C₃-C₈ cycloalkyl, C₃-C₆ cycloalkyl, or C₅-C₆ cycloalkyl), R⁵⁸-substituted or unsubstituted heterocycloalkyl (e.g., 3 to 8 membered heterocycloalkyl, 3 to 6 membered heterocycloalkyl, or 5 to 6 membered heterocycloalkyl), R⁵⁸-substituted or unsubstituted aryl (e.g., C₆-C₁₀ aryl, C₁₀ aryl, or phenyl), or R⁵⁸-substituted or unsubstituted heteroaryl (e.g., 5 to 10 membered heteroaryl, 5 to 9 membered heteroaryl, or 5 to 6 membered heteroaryl).

In embodiments, R^{30C} is R⁵⁸-substituted or unsubstituted alkyl (e.g., C₁-C₈ alkyl, C₁-C₆ alkyl, or C₁-C₄ alkyl). In embodiments, R^{30C} is R⁵⁸-substituted alkyl (e.g., C₁-C₈ alkyl, C₁-C₆ alkyl, or C₁-C₄ alkyl). In embodiments, R^{30C} is an unsubstituted alkyl (e.g., C₁-C₈ alkyl, C₁-C₆ alkyl, or C₁-C₄ alkyl).

In embodiments, R^{30C} is R⁵⁸-substituted or unsubstituted heteroalkyl (e.g., 2 to 8 membered heteroalkyl, 2 to 6 membered heteroalkyl, or 2 to 4 membered heteroalkyl). In embodiments, R^{30C} is R⁵⁸-substituted heteroalkyl (e.g., 2 to 8 membered heteroalkyl, 2 to 6 membered heteroalkyl, or 2 to 4 membered heteroalkyl). In embodiments, R^{30C} is an unsubstituted heteroalkyl (e.g., 2 to 8 membered heteroalkyl, 2 to 6 membered heteroalkyl, or 2 to 4 membered heteroalkyl).

In embodiments, R^{30C} is R⁵⁸-substituted or unsubstituted cycloalkyl (e.g., C₃-C₈ cycloalkyl, C₃-C₆ cycloalkyl, or C₅-C₆ cycloalkyl). In embodiments, R^{30C} is R⁵⁸-substituted cycloalkyl (e.g., C₃-C₈ cycloalkyl, C₃-C₆ cycloalkyl, or C₅-C₆ cycloalkyl). In embodiments, R^{30C} is an unsubstituted cycloalkyl (e.g., C₃-C₈ cycloalkyl, C₃-C₆ cycloalkyl, or C₅-C₆ cycloalkyl).

In embodiments, R^{30C} is R⁵⁸-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R^{30C} is R⁵⁸-substituted heterocycloalkyl (e.g., 3 to 8 membered heterocycloalkyl, 3 to 6 membered heterocycloalkyl, or 5 to 6 membered heterocycloalkyl). In embodiments, R^{30C} is an unsubstituted heterocycloalkyl (e.g., 3 to 8 membered heterocycloalkyl, 3 to 6 membered heterocycloalkyl, or 5 to 6 membered heterocycloalkyl).

In embodiments, R^{30C} is R⁵⁸-substituted or unsubstituted aryl (e.g., C₆-C₁₀ aryl, C₁₀ aryl, or phenyl). In embodiments, R^{30C} is R⁵⁸-substituted aryl (e.g., C₆-C₁₀ aryl, C₁₀ aryl, or 5 phenyl). In embodiments, R^{30C} is an unsubstituted aryl (e.g., C₆-C₁₀ aryl, C₁₀ aryl, or phenyl).

In embodiments, R^{30C} is R⁵⁸-substituted or unsubstituted heteroaryl (e.g., 5 to 10 membered heteroaryl, 5 to 9 membered heteroaryl, or 5 to 6 membered heteroaryl). In embodiments, R^{30C} is R⁵⁸-substituted heteroaryl (e.g., 5 to 10 membered heteroaryl, 5 to 9 membered heteroaryl, or 5 to 6 membered heteroaryl). In embodiments, R^{30C} is an unsubstituted heteroaryl (e.g., 5 to 10 membered heteroaryl, 5 to 9 membered heteroaryl, or 5 to 6 membered heteroaryl).

In embodiments, compound A2 has the formula:

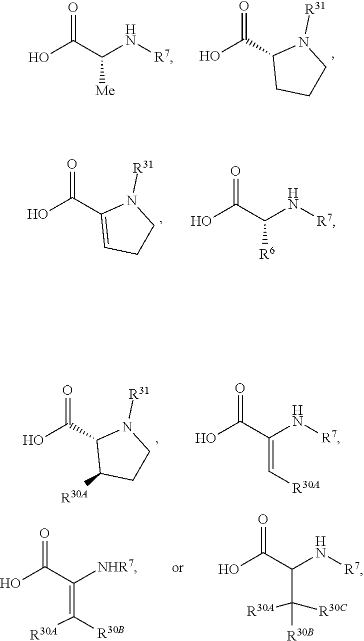

wherein R⁶, R⁷, R³¹, R^{30A}, R^{30B}, and R^{30C} are as described herein.

In embodiments, compound A2 has the formula:

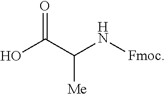

In embodiments, compound A2 has the formula:

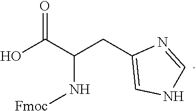

In embodiments, compound A2 has the formula:

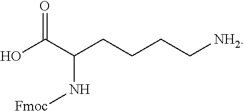

In embodiments, compound A2 has the formula:

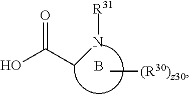

wherein R³¹ and R³⁰ are as described herein and z30 is an integer from 0 to 6. In embodiments, when R⁶ and R⁷ are joined to form a substituted or unsubstituted heterocycloalkyl or substituted or unsubstituted heteroaryl, it may be referred to herein as Ring B. Therefore, Ring B is a heterocycloalkyl or heteroaryl.

In embodiments, compound A2 has the formula:

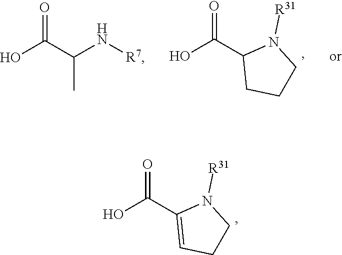

wherein R⁷ and R³¹ are as described herein. In embodiments, z30 is 0. In embodiments, z30 is 1. In embodiments, z30 is 2. In embodiments, z30 is 3. In embodiments, z30 is 4. In embodiments, z30 is 5. In embodiments, z30 is 6.

In embodiments, compound A2 has the formula:

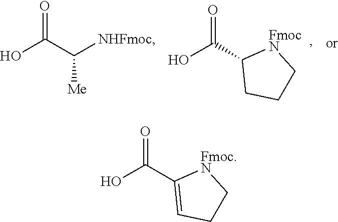

In embodiments, compound A2 has the formula:

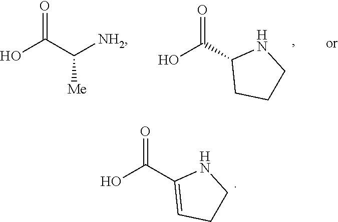 [SMILES: C[C@@H](N)C(=O)O ; O=C(O)[C@H]1CCCN1 ; "," ; "or" ; O=C(O)C1=CCCN1]

In embodiments, compound A2 has the formula:

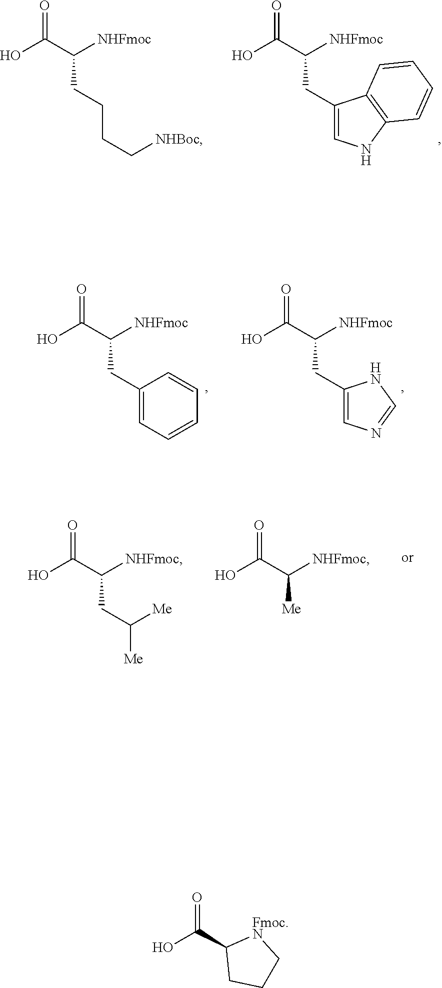

In embodiments, compound A3 has the formula:

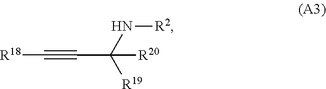

wherein R² is hydrogen or unsubstituted C₁-C₃ alkyl; R¹⁸, R¹⁹, and R²⁰ are independently hydrogen, substituted or unsubstituted alkyl, substituted or unsubstituted heteroalkyl. In embodiments, compound A3 has the formula:

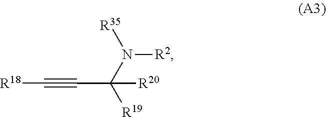

wherein R³⁵ is a leaving group. In embodiments, the hydrogen attached to the nitrogen may leave upon reaction with compound A4 (e.g., the methoxy moiety). In embodiments, R¹⁸, R¹⁹, and R²⁰ are independently hydrogen.

In embodiments, R¹⁸, R¹⁹, and R²⁰ are independently substituted (e.g., substituted with a substituent group, a size-limited substituent group, or lower substituent group) or unsubstituted alkyl. In embodiments, R¹⁸, R¹⁹, and R²⁰ are independently substituted (e.g., substituted with a substituent group, a size-limited substituent group, or lower substituent group) alkyl. In embodiments, R¹⁸, R¹⁹, and R²⁰ are independently unsubstituted alkyl. In embodiments, R¹⁸, R¹⁹, and R²⁰ are independently substituted or unsubstituted alkyl (e.g., C₁-C₈, C₁-C₆, C₁-C₄, or C₁-C₂). In embodiments, R¹⁸, R¹⁹, and R²⁰ are independently substituted alkyl (e.g., C₁-C₈, C₁-C₆, C₁-C₄, or C₁-C₂). In embodiments, R¹⁸, R¹⁹, and R²⁰ are independently unsubstituted alkyl (e.g., C₁-C₈, C₁-C₆, C₁-C₄, or C₁-C₂).

In embodiments, R¹⁸, R¹⁹, and R²⁰ are independently substituted (e.g., substituted with a substituent group, a size-limited substituent group, or lower substituent group) or unsubstituted heteroalkyl. In embodiments, R¹⁸, R¹⁹, and R²⁰ are independently substituted (e.g., substituted with a substituent group, a size-limited substituent group, or lower substituent group) heteroalkyl. In embodiments, R¹⁸, R¹⁹, and R²⁰ are independently an unsubstituted heteroalkyl. In embodiments, R¹⁸, R¹⁹, and R²⁰ are independently substituted or unsubstituted heteroalkyl (e.g., 2 to 8 membered, 2 to 6 membered, 4 to 6 membered, 2 to 3 membered, or 4 to 5 membered). In embodiments, R¹⁸, R¹⁹, and R²⁰ are independently substituted heteroalkyl (e.g., 2 to 8 membered, 2 to 6 membered, 4 to 6 membered, 2 to 3 membered, or 4 to 5 membered). In embodiments, R¹⁸, R¹⁹, and R²⁰ are independently an unsubstituted heteroalkyl (e.g., 2 to 8 membered, 2 to 6 membered, 4 to 6 membered, 2 to 3 membered, or 4 to 5 membered).

In embodiments, R¹⁸ is R⁵⁴-substituted or unsubstituted alkyl (e.g., C₁-C₈ alkyl, C₁-C₆ alkyl, or C₁-C₄ alkyl). In embodiments, R¹⁸ is R⁵⁴-substituted alkyl (e.g., C₁-C₈ alkyl, C₁-C₆ alkyl, or C₁-C₄ alkyl). In embodiments, R¹⁸ is an unsubstituted alkyl (e.g., C₁-C₈ alkyl, C₁-C₆ alkyl, or C₁-C₄ alkyl).

In embodiments, R¹⁸ is R⁵⁴-substituted or unsubstituted heteroalkyl (e.g., 2 to 8 membered heteroalkyl, 2 to 6 membered heteroalkyl, or 2 to 4 membered heteroalkyl). In embodiments, R¹⁸ is R⁵⁴-substituted heteroalkyl (e.g., 2 to 8 membered heteroalkyl, 2 to 6 membered heteroalkyl, or 2 to 4 membered heteroalkyl). In embodiments, R¹⁸ is an unsubstituted heteroalkyl (e.g., 2 to 8 membered heteroalkyl, 2 to 6 membered heteroalkyl, or 2 to 4 membered heteroalkyl).

In embodiments, R¹⁹ is R⁵⁵-substituted or unsubstituted alkyl (e.g., C₁-C₈ alkyl, C₁-C₆ alkyl, or C₁-C₄ alkyl). In embodiments, R¹⁹ is R⁵⁵-substituted alkyl (e.g., C₁-C₈ alkyl, C₁-C₆ alkyl, or C₁-C₄ alkyl). In embodiments, R¹⁹ is an unsubstituted alkyl (e.g., C₁-C₈ alkyl, C₁-C₆ alkyl, or C₁-C₄ alkyl).

In embodiments, R¹⁹ is R⁵⁵-substituted or unsubstituted heteroalkyl (e.g., 2 to 8 membered heteroalkyl, 2 to 6 membered heteroalkyl, or 2 to 4 membered heteroalkyl). In embodiments, R¹⁹ is R⁵⁵-substituted heteroalkyl (e.g., 2 to 8 membered heteroalkyl, 2 to 6 membered heteroalkyl, or 2 to 4 membered heteroalkyl). In embodiments, R¹⁹ is an unsubstituted heteroalkyl (e.g., 2 to 8 membered heteroalkyl, 2 to 6 membered heteroalkyl, or 2 to 4 membered heteroalkyl).

In embodiments, R²⁰ is R⁵⁶-substituted or unsubstituted alkyl (e.g., C₁-C₈ alkyl, C₁-C₆ alkyl, or C₁-C₄ alkyl). In embodiments, R²⁰ is R⁵⁶-substituted alkyl (e.g., C₁-C₈ alkyl, C₁-C₆ alkyl, or C₁-C₄ alkyl). In embodiments, R²⁰ is an unsubstituted alkyl (e.g., C₁-C₈ alkyl, C₁-C₆ alkyl, or C₁-C₄ alkyl).

In embodiments, R²⁰ is R⁵⁶-substituted or unsubstituted heteroalkyl (e.g., 2 to 8 membered heteroalkyl, 2 to 6 membered heteroalkyl, or 2 to 4 membered heteroalkyl). In embodiments, R²⁰ is R⁵⁶-substituted heteroalkyl (e.g., 2 to 8 membered heteroalkyl, 2 to 6 membered heteroalkyl, or 2 to 4 membered heteroalkyl). In embodiments, R²⁰ is an unsubstituted heteroalkyl (e.g., 2 to 8 membered heteroalkyl, 2 to 6 membered heteroalkyl, or 2 to 4 membered heteroalkyl).

In embodiments, compound A3 has the formula

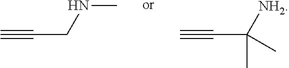

embodiments, compound A3 has the formula:

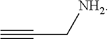

In embodiments, compound A3 has the formula:

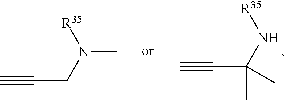

wherein R³⁵ is as described herein. In embodiments, compound A3 has the formula:

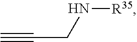

wherein R³⁵ is as described herein.

In embodiments, compound A4 has the formula:

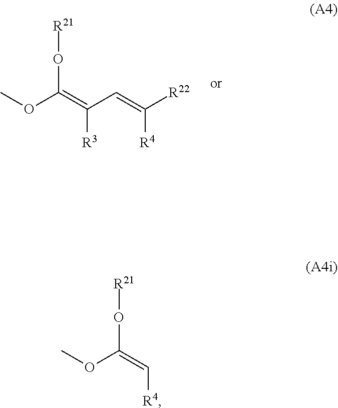

wherein R³ and R⁴ are as described herein, including embodiments; R²¹ is hydrogen or a protecting group; and R²² is hydrogen, substituted or unsubstituted alkyl, substituted or unsubstituted heteroalkyl. In embodiments, R²² is hydrogen.

In embodiments, R²² is R⁵⁷-substituted or unsubstituted alkyl (e.g., C₁-C₈ alkyl, C₁-C₆ alkyl, or C₁-C₄ alkyl). In embodiments, R²² is R⁵⁷-substituted alkyl (e.g., C₁-C₈ alkyl, C₁-C₆ alkyl, or C₁-C₄ alkyl). In embodiments, R²² is an unsubstituted alkyl (e.g., C₁-C₈ alkyl, C₁-C₆ alkyl, or C₁-C₄ alkyl).

In embodiments, R²² is R⁵⁷-substituted or unsubstituted heteroalkyl (e.g., 2 to 8 membered heteroalkyl, 2 to 6 membered heteroalkyl, or 2 to 4 membered heteroalkyl). In embodiments, R²² is R⁵⁷-substituted heteroalkyl (e.g., 2 to 8 membered heteroalkyl, 2 to 6 membered heteroalkyl, or 2 to 4 membered heteroalkyl). In embodiments, R²² is an unsubstituted heteroalkyl (e.g., 2 to 8 membered heteroalkyl, 2 to 6 membered heteroalkyl, or 2 to 4 membered heteroalkyl).

In embodiments, R²² is hydrogen. In embodiments, R²² is substituted (e.g., substituted with a substituent group, a size-limited substituent group, or lower substituent group) or unsubstituted alkyl. In embodiments, R²² is substituted (e.g., substituted with a substituent group, a size-limited substituent group, or lower substituent group) alkyl. In embodiments, R²² is unsubstituted alkyl. In embodiments, R²² is substituted or unsubstituted alkyl (e.g., C₁-C₂₂, C₁-C₅, C₁-C₄, or C₁-C₂). In embodiments, R²² is substituted alkyl (e.g., C₁-C₂₂, C₁-C₆, C₁-C₄, or C₁-C₂). In embodiments, R²² is unsubstituted alkyl (e.g., C₁-C₂₂, C₁-C₆, C₁-C₄, or C₁-C₂).

In embodiments, R²² is substituted (e.g., substituted with a substituent group, a size-limited substituent group, or lower substituent group) or unsubstituted heteroalkyl. In embodiments, R²² is substituted (e.g., substituted with a substituent group, a size-limited substituent group, or lower substituent group) heteroalkyl. In embodiments, R²² is unsubstituted heteroalkyl. In embodiments, R²² is substituted or unsubstituted heteroalkyl (e.g., 2 to 22 membered, 2 to 6 membered, 4 to 6 membered, 2 to 3 membered, or 4 to 5 membered). In embodiments, R²² is substituted heteroalkyl (e.g., 2 to 22 membered, 2 to 6 membered, 4 to 6 membered, 2 to 3 membered, or 4 to 5 membered). In embodiments, R²² is an unsubstituted heteroalkyl (e.g., 2 to 22 membered, 2 to 6 membered, 4 to 6 membered, 2 to 3 membered, or 4 to 5 membered).

In embodiments, compound A5 has the formula:

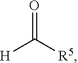

wherein R⁵ is halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, the atom or chemical moieties to which R⁵ in compound A5 and the atom or chemical moieties to which R⁴ in compound A4 are attached form a complementary reactive group pair (e.g., bioconjugate reactive moieties). For example, the carbonyl in A5 and the —C═C=moiety to which R⁴ is attached may be complementary reactive group pairs.

In embodiments, R⁵ is an unsubstituted C₄ alkyl. In embodiments, R⁵ is an unsubstituted C₃ alkyl. In embodiments, R⁵ is isopropyl. In embodiments, R⁵ is t-butyl. In embodiments, R⁵ is

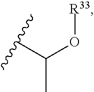

wherein R³³ is as described herein. In embodiments, R⁵ is

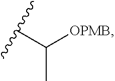

wherein ‘PMB’ is 4-methoxybenzyl ether. In embodiments, R⁵ is

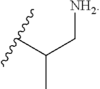 [SMILES: *C(C)CN]

In embodiments, R⁵ is

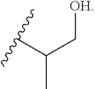 [SMILES: *C(C)CO]

In embodiments, R⁵ is

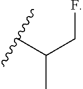 [SMILES: *C(C)CF]

In embodiments, R⁵ is

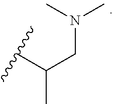 [SMILES: *C(C)CN(C)C]

In embodiments, R⁵ is

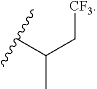 [SMILES: *C(C)CC(F)(F)F]

In embodiments, R⁵ is

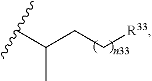

wherein n33 is an integer from 0 to 20. In embodiments, n33 is an integer from 0 to 4.

In embodiments, compound B has the formula:

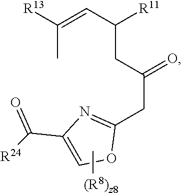

wherein R¹³, R¹¹, R⁸, z8, and R²⁴ are as described herein. In embodiments, compound B has the formula:

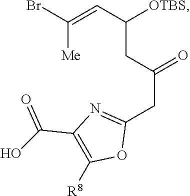

wherein R⁸ is a protecting group (e.g., TMS).

In embodiments, compound B has the formula:

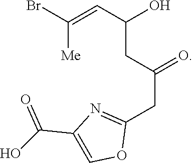 [SMILES: C/C(Br)=C\C(O)CC(=O)Cc1nc(C(=O)O)co1]

In embodiments, compound B has the formula:

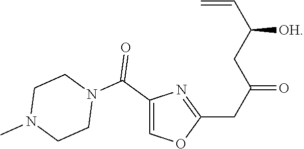 [SMILES: C=C[C@@H](O)CC(=O)Cc1nc(C(=O)N2CCN(C)CC2)co1]

In embodiments, compound B2 has the formula:

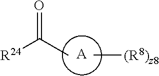

wherein Ring A, R⁸, and z8 are as described herein and R²⁴ is a leaving group.

In embodiments compound B2 has the formula:

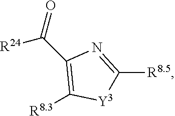

wherein R²⁴ is as described herein and Y³ is —S—, —O—, or —NR^{8.4}-. R^{8.3}, R^{8.4}, and R^{8.5} are each R⁸ at a fixed position on the attached ring. R^{8.3}, R^{8.4}, and R^{8.5} may be hydrogen or a substituent of R⁸ described herein, including in any aspect, embodiment, example, figure, or claim. In embodiments, R^{8.4} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —CCl₃, —COOH, —CH₂COOH, —CONH₂, —OH, —SH, substituted or unsubstituted alkyl, or substituted or unsubstituted heteroalkyl; and R^{8.3} and R^{8.5} are independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, or substituted or unsubstituted heteroalkyl. In embodiments, R^{8.3} and R^{8.5} are independently hydrogen, halogen, —CCl₂, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₂, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, or substituted or unsubstituted heteroalkyl.

In embodiments, R^{8.3}, R^{8.4} and R^{8.5} are each independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{8.3}, R^{8.4}, and R^{8.5} are each independently substituted (e.g., substituted with a substituent group, a size-limited substituent group, or lower substituent group) or unsubstituted alkyl. In embodiments, R^{8.3}, R^{8.4}, and R^{8.5} are each independently substituted (e.g., substituted with a substituent group, a size-limited substituent group, or lower substituent group) alkyl. In embodiments, R^{8.3}, R^{8.4}, and R^{8.5} are each independently unsubstituted alkyl. In embodiments, R^{8.3}, R^{8.4}, and R^{8.5} are each independently substituted or unsubstituted alkyl (e.g., C₁-C₈, C₁-C₆, C₁-C₄, or C₁-C₂). In embodiments, R^{8.3}, R^{8.4}, and R^{8.5} are each independently substituted alkyl (e.g., C₁-C₈, C₁-C₆, C₁-C₄, or C₁-C₂). In embodiments, R^{8.3}, R^{8.4}, and R^{8.5} are each independently unsubstituted alkyl (e.g., C₁-C₈, C₁-C₆, C₁-C₄, or C₁-C₂).

In embodiments, R^{8.3}, R^{8.4}, and R^{8.5} are each independently substituted (e.g., substituted with a substituent group, a size-limited substituent group, or lower substituent group) or unsubstituted heteroalkyl. In embodiments, R^{8.3}, R^{8.4}, and R^{8.5} are each independently substituted (e.g., substituted with a substituent group, a size-limited substituent group, or lower substituent group) heteroalkyl. In embodiments, R^{8.3}, R^{8.4}, and R^{8.5} are each independently unsubstituted heteroalkyl. In embodiments, R^{8.3}, R^{8.4}, and R^{8.5} are each independently substituted or unsubstituted heteroalkyl (e.g., 2 to 8 membered, 2 to 6 membered, 4 to 6 membered, 2 to 3 membered, or 4 to 5 membered). In embodiments, R^{8.3}, R^{8.4} and R^{8.5} are each independently substituted heteroalkyl (e.g., 2 to 8 membered, 2 to 6 membered, 4 to 6 membered, 2 to 3 membered, or 4 to 5 membered). In embodiments, R^{8.3}, R^{8.4} and R^{8.5} are each independently an unsubstituted heteroalkyl (e.g., 2 to 8 membered, 2 to 6 membered, 4 to 6 membered, 2 to 3 membered, or 4 to 5 membered).

In embodiments, R^{8.3}, R^{8.4}, and R^{8.5} are each independently substituted (e.g., substituted with a substituent group, a size-limited substituent group, or lower substituent group) or unsubstituted cycloalkyl. In embodiments, R^{8.3}, R^{8.4}, and R^{8.5} are each independently substituted (e.g., substituted with a substituent group, a size-limited substituent group, or lower substituent group) cycloalkyl. In embodiments, R^{8.3}, R^{8.4}, and R^{8.5} are each independently an unsubstituted cycloalkyl. In embodiments, R^{8.3}, R^{8.4}, and R^{8.5} are each independently substituted or unsubstituted cycloalkyl (e.g., C₃-C₈, C₃-C₆, C₄-C₆, or C₅-C₆). In embodiments, R^{8.3}, R^{8.4}, and R^{8.5} are each independently substituted cycloalkyl (e.g., C₃-C₈, C₃-C₆, C₄-C₆, or C₅-C₆). In embodiments, R^{8.3}, R^{8.4}, and R^{8.5} are each independently unsubstituted cycloalkyl (e.g., C₃-C₈, C₃-C₅, C₄-C₆, or C₅-C₆).

In embodiments, R^{8.3}, R^{8.4}, and R^{8.5} are each independently substituted (e.g., substituted with a substituent group, a size-limited substituent group, or lower substituent group) or unsubstituted heterocycloalkyl. In embodiments, R^{8.3}, R^{8.4} and R^{8.5} are each independently substituted (e.g., substituted with a substituent group, a size-limited substituent group, or lower substituent group) heterocycloalkyl. In embodiments, R^{8.3}, R^{8.4}, and R^{8.5} are each independently an unsubstituted heterocycloalkyl. In embodiments, R^{8.3}, R^{8.4}, and R^{8.5} are each independently substituted or unsubstituted heterocycloalkyl (e.g., 3 to 8 membered, 3 to 6 membered, 4 to 6 membered, 4 to 5 membered, or 5 to 6 membered). In embodiments, R^{8.3}, R^{8.4}, and R^{8.5} independently substituted heterocycloalkyl (e.g., 3 to 8 membered, 3 to 6 membered, 4 to 6 membered, 4 to 5 membered, or 5 to 6 membered). In embodiments, R^{8.3}, R^{8.4}, and R^{8.5} are each independently an unsubstituted heterocycloalkyl (e.g., 3 to 8 membered, 3 to 6 membered, 4 to 6 membered, 4 to 5 membered, or 5 to 6 membered).

In embodiments, R^{8.3}, R^{8.4}, and R^{8.5} are each independently substituted (e.g., substituted with a substituent group, a size-limited substituent group, or lower substituent group) or unsubstituted aryl. In embodiments, R^{8.3}, R^{8.4}, and R^{8.5} are each independently substituted (e.g., substituted with a substituent group, a size-limited substituent group, or lower substituent group) aryl. In embodiments, R^{8.3}, R^{8.4}, and R^{8.5} are each independently an unsubstituted aryl. In embodiments, R^{8.3}, R^{8.4}, and R^{8.5} are each independently substituted or unsubstituted aryl (e.g., C₆-C₁₀ or phenyl). In embodiments, R^{8.3}, R^{8.4}, and R^{8.5} are each independently substituted aryl (e.g., C₆-C₁₀ or phenyl). In embodiments, R^{8.3}, R^{8.4}, and R^{8.5} are each independently an unsubstituted aryl (e.g., C₆-C₁₀ or phenyl).

In embodiments, R^{8.3}, R^{8.4}, and R^{8.5} are each independently substituted (e.g., substituted with a substituent group, a size-limited substituent group, or lower substituent group) or unsubstituted heteroaryl. In embodiments, R^{8.3}, R^{8.4}, and R^{8.5} are each independently substituted (e.g., substituted with a substituent group, a size-limited substituent group, or lower substituent group) heteroaryl. In embodiments, R^{8.3}, R^{8.4}, and R^{8.5} are each independently an unsubstituted heteroaryl. In embodiments, R^{8.3}, R^{8.4}, and R^{8.5} are each independently substituted or unsubstituted heteroaryl (e.g., 5 to 10 membered, 5 to 9 membered, or 5 to 6 membered). In embodiments, R^{8.3}, R^{8.4}, and R^{8.5} are each independently substituted heteroaryl (e.g., 5 to 10 membered, 5 to 9 membered, or 5 to 6 membered). In embodiments, R^{8.3}, R^{8.4}, and R^{8.5} are each independently an unsubstituted heteroaryl (e.g., 5 to 10 membered, 5 to 9 membered, or 5 to 6 membered).

R^{8.3}, R^{8.4}, and R^{8.5} are each hydrogen or R⁸ at a fixed position on the attached ring. R^{8.3}, R^{8.4}, and R^{8.5} may be a substituent of R⁸ described herein, including in any aspect, embodiment, example, figure, or claim. In embodiments, R^{8.4} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —CCl₃, —COOH, —CH₂COOH, —CONH₂, —OH, —SH, substituted or unsubstituted alkyl, or substituted or unsubstituted heteroalkyl. In embodiments, R^{8.4} is a protecting group. In embodiments, R^{8.4} is TMS. In embodiments, R^{8.3} and R^{8.5} are independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, or substituted or unsubstituted heteroalkyl.

In embodiments compound B2 has the formula:

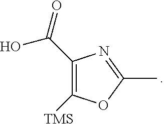

In embodiments compound B2 has the formula:

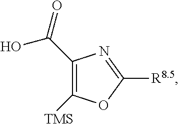

wherein R^{8.5} is as described herein. In embodiments compound B2 has the formula:

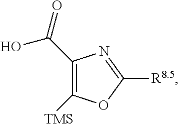

wherein R^{8.5} is as described herein.

In embodiments compound B2 has the formula:

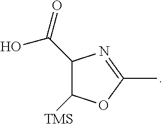

In embodiments compound B2 has the formula:

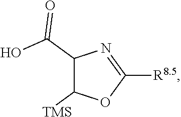

wherein R^{8.5} is as described herein. In embodiments compound B2 has the formula:

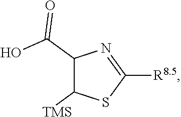

wherein R^{8.5} is as described herein. In embodiments compound B2 has the formula:

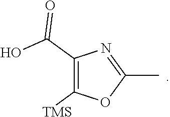

In embodiments compound B2 has the formula:

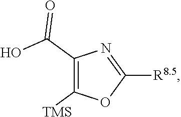

wherein R^{8.5} is as described herein. In embodiments compound B2 has the formula:

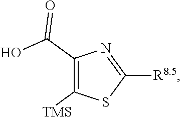

wherein R^{8.5} is as described herein. In embodiments compound B2 has the formula:

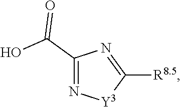

wherein Y³ and R^{8.5} is as described herein. In embodiments compound B2 has the formula:

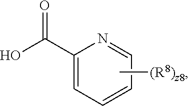

wherein z8 and R⁸ is as described herein. In embodiments compound B2 has the formula:

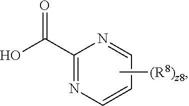

wherein z8 and R⁸ is as described herein. In embodiments compound B2 has the formula:

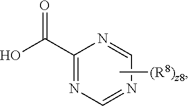

wherein z8 and R⁸ is as described herein. In embodiments compound B2 has the formula:

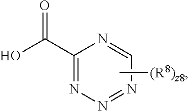

wherein z8 and R⁸ is as described herein. In embodiments compound B2 has the formula:

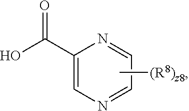

wherein z8 and R⁸ is as described herein. In embodiments compound B2 has the formula:

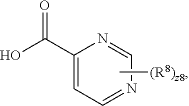

wherein z8 and R⁸ is as described herein. In embodiments compound B2 has the formula:

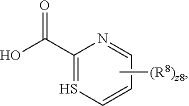

wherein z8 and R⁸ is as described herein. In embodiments compound B2 has the formula:

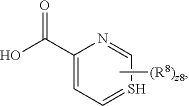

wherein z8 and R⁸ is as described herein. In embodiments compound B2 has the formula:

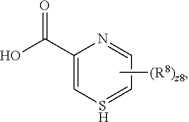

wherein z8 and R⁸ is as described herein.

In embodiments, compound B3 has the formula:

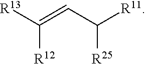

wherein R¹¹, R¹², and R¹³ are as described herein, including embodiments, and R²⁵ is a leaving group. In embodiments, R¹² is substituted or unsubstituted phenyl. In embodiments, compound B3 has the formula:

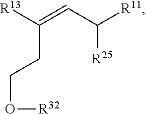

wherein R¹¹, R²⁵, and R¹³ are as described herein, including embodiments.

R³² is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, or a protecting group (e.g., TBS). In embodiments, R³² is hydrogen, —NH₂, or —COOH.

In embodiments, R³² is substituted (e.g., substituted with a substituent group, a size-limited substituent group, or lower substituent group) or unsubstituted alkyl. In embodiments, R³² is substituted (e.g., substituted with a substituent group, a size-limited substituent group, or lower substituent group) alkyl. In embodiments, R³² is unsubstituted alkyl. In embodiments, R³² is substituted or unsubstituted alkyl (e.g., C₁-C₃₂, C₁-C₆, C₁-C₄, or C₁-C₂). In embodiments, R³² is substituted alkyl (e.g., C₁-C₃₂, C₁-C₅, C₁-C₄, or C₁-C₂). In embodiments, R³² is unsubstituted alkyl (e.g., C₁-C₃₂, C₁-C₆, C₁-C₄, or C₁-C₂).

In embodiments, R³² is substituted (e.g., substituted with a substituent group, a size-limited substituent group, or lower substituent group) or unsubstituted heteroalkyl. In embodiments, R³² is substituted (e.g., substituted with a substituent group, a size-limited substituent group, or lower substituent group) heteroalkyl. In embodiments, R³² is unsubstituted heteroalkyl. In embodiments, R³² is substituted or unsubstituted heteroalkyl (e.g., 2 to 32 membered, 2 to 6 membered, 4 to 6 membered, 2 to 3 membered, or 4 to 5 membered). In embodiments, R³² is substituted heteroalkyl (e.g., 2 to 32 membered, 2 to 6 membered, 4 to 6 membered, 2 to 3 membered, or 4 to 5 membered). In embodiments, R³² is an unsubstituted heteroalkyl (e.g., 2 to 32 membered, 2 to 6 membered, 4 to 6 membered, 2 to 3 membered, or 4 to 5 membered).

In embodiments, R³² is R⁶⁰-substituted or unsubstituted alkyl (e.g., C₁-C₈ alkyl, C₁-C₆ alkyl, or C₁-C₄ alkyl), R⁶⁰-substituted or unsubstituted heteroalkyl (e.g., 2 to 8 membered heteroalkyl, 2 to 6 membered heteroalkyl, or 2 to 4 membered heteroalkyl), R⁶⁰-substituted or unsubstituted cycloalkyl (e.g., C₃-C₈ cycloalkyl, C₃-C₆ cycloalkyl, or C₅-C₆ cycloalkyl), R⁶⁰-substituted or unsubstituted heterocycloalkyl (e.g., 3 to 8 membered heterocycloalkyl, 3 to 6 membered heterocycloalkyl, or 5 to 6 membered heterocycloalkyl), R⁶⁰-substituted or unsubstituted aryl (e.g., C₆-C₁₀ aryl, C₁₀ aryl, or phenyl), or R⁶⁰-substituted or unsubstituted heteroaryl (e.g., 5 to 10 membered heteroaryl, 5 to 9 membered heteroaryl, or 5 to 6 membered heteroaryl).

In embodiments, R³² is R⁶⁰-substituted or unsubstituted alkyl (e.g., C₁-C₈ alkyl, C₁-C₆ alkyl, or C₁-C₄ alkyl). In embodiments, R³² is R⁶⁰-substituted alkyl (e.g., C₁-C₈ alkyl, C₁-C₆ alkyl, or C₁-C₄ alkyl). In embodiments, R³² is an unsubstituted alkyl (e.g., C₁-C₈ alkyl, C₁-C₆ alkyl, or C₁-C₄ alkyl).

In embodiments, R³² is R⁶⁰-substituted or unsubstituted heteroalkyl (e.g., 2 to 8 membered heteroalkyl, 2 to 6 membered heteroalkyl, or 2 to 4 membered heteroalkyl). In embodiments, R³² is R⁶⁰-substituted heteroalkyl (e.g., 2 to 8 membered heteroalkyl, 2 to 6 membered heteroalkyl, or 2 to 4 membered heteroalkyl). In embodiments, R³² is an unsubstituted heteroalkyl (e.g., 2 to 8 membered heteroalkyl, 2 to 6 membered heteroalkyl, or 2 to 4 membered heteroalkyl).

In embodiments, R³² is R⁶⁰-substituted or unsubstituted cycloalkyl (e.g., C₃-C₈ cycloalkyl, C₃-C₆ cycloalkyl, or C₅-C₆ cycloalkyl). In embodiments, R³² is R⁶⁰-substituted cycloalkyl (e.g., C₃-C₈ cycloalkyl, C₃-C₆ cycloalkyl, or C₅-C₆ cycloalkyl). In embodiments, R³² is an unsubstituted cycloalkyl (e.g., C₃-C₈ cycloalkyl, C₃-C₆ cycloalkyl, or C₅-C₆ cycloalkyl).

In embodiments, R³² is R⁶⁰-substituted or unsubstituted heterocycloalkyl (e.g., 3 to 8 membered heterocycloalkyl, 3 to 6 membered heterocycloalkyl, or 5 to 6 membered heterocycloalkyl). In embodiments, R³² is R⁶⁰-substituted heterocycloalkyl (e.g., 3 to 8 membered heterocycloalkyl, 3 to 6 membered heterocycloalkyl, or 5 to 6 membered heterocycloalkyl). In embodiments, R³² is an unsubstituted heterocycloalkyl (e.g., 3 to 8 membered heterocycloalkyl, 3 to 6 membered heterocycloalkyl, or 5 to 6 membered heterocycloalkyl).

In embodiments, R³² is R⁶⁰-substituted or unsubstituted aryl (e.g., C₆-C₁₀ aryl, C₁₀ aryl, or phenyl). In embodiments, R³² is R⁶⁰-substituted aryl (e.g., C₆-C₁₀ aryl, C₁₀ aryl, or phenyl). In embodiments, R³² is an unsubstituted aryl (e.g., C₆-C₁₀ aryl, C₁₀ aryl, or phenyl).

In embodiments, R³² is R⁶⁰-substituted or unsubstituted heteroaryl (e.g., 5 to 10 membered heteroaryl, 5 to 9 membered heteroaryl, or 5 to 6 membered heteroaryl). In embodiments, R³² is R⁶⁰-substituted heteroaryl (e.g., 5 to 10 membered heteroaryl, 5 to 9 membered heteroaryl, or 5 to 6 membered heteroaryl). In embodiments, R³² is an unsubstituted heteroaryl (e.g., 5 to 10 membered heteroaryl, 5 to 9 membered heteroaryl, or 5 to 6 membered heteroaryl).

In embodiments, compound B3 has the formula:

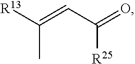

wherein R¹³ and R²⁵ are leaving groups. In embodiments, compound B3 has the formula:

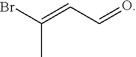

In embodiments, compound B4 has the formula:

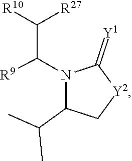

wherein R⁹ and R¹⁰ are as described herein, including embodiments, and R²⁷ is a leaving group. In embodiments, compound B4 has the formula:

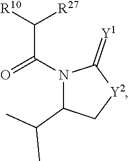

wherein R¹⁰ is as described herein, including embodiments, and R²⁷ is a leaving group. The symbol Y¹ is ═O or S. The symbol Y² is —O— or —S—. In embodiments, compound B4 has the formula:

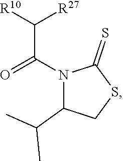

wherein R¹⁰ is as described herein, including embodiments, and R²⁷ is a leaving group. In embodiments, compound B4 has the formula:

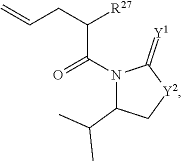

wherein R²⁷, Y¹, and Y² are as described herein, including embodiments. In embodiments, compound B4 has the formula:

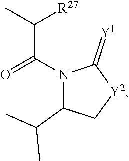

wherein R²⁷, Y¹, and Y² are as described herein, including embodiments. In embodiments, compound B4 has the formula:

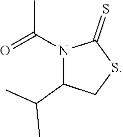

In embodiments, compound D has the formula:

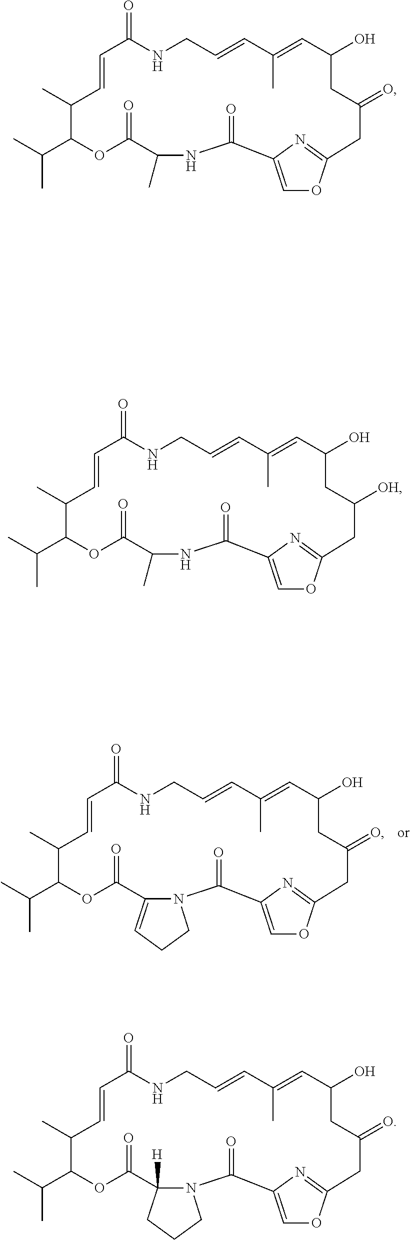 [SMILES: CC1=C\C(O)CC(=O)Cc2nc(co2)C(=O)NC(C)C(=O)OC(C(C)C)C(C)/C=C/C(=O)NC\C=C\1 ; CC1=C\C(O)CC(O)Cc2nc(co2)C(=O)NC(C)C(=O)OC(C(C)C)C(C)/C=C/C(=O)NC\C=C\1 ; CC1=C\C(O)CC(=O)Cc2nc(co2)C(=O)N2CCC=C2C(=O)OC(C(C)C)C(C)/C=C/C(=O)NC\C=C\1 ; CC1=C\C(O)CC(=O)Cc2nc(co2)C(=O)N2CCC[C@@H]2C(=O)OC(C(C)C)C(C)/C=C/C(=O)NC\C=C\1]

In embodiments, compound D has the formula:

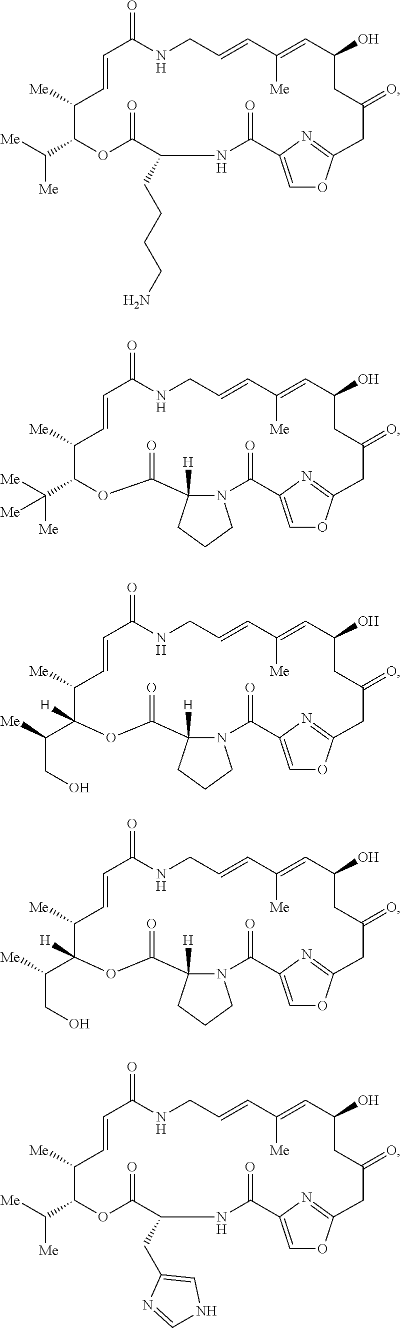 [SMILES: CC1=C\[C@@H](O)CC(=O)Cc2nc(co2)C(=O)N[C@H](CCCCN)C(=O)O[C@H](C(C)C)[C@H](C)/C=C/C(=O)NC\C=C\1 ; CC1=C\[C@@H](O)CC(=O)Cc2nc(co2)C(=O)N2CCC[C@@H]2C(=O)O[C@H](C(C)(C)C)[C@H](C)/C=C/C(=O)NC\C=C\1 ; CC1=C\[C@@H](O)CC(=O)Cc2nc(co2)C(=O)N2CCC[C@@H]2C(=O)O[C@H]([C@H](C)CO)[C@H](C)/C=C/C(=O)NC\C=C\1 ; CC1=C\[C@@H](O)CC(=O)Cc2nc(co2)C(=O)N2CCC[C@@H]2C(=O)O[C@H]([C@@H](C)CO)[C@H](C)/C=C/C(=O)NC\C=C\1 ; CC1=C\[C@@H](O)CC(=O)Cc2nc(co2)C(=O)N[C@H](Cc2c[nH]cn2)C(=O)O[C@H](C(C)C)[C@H](C)/C=C/C(=O)NC\C=C\1]

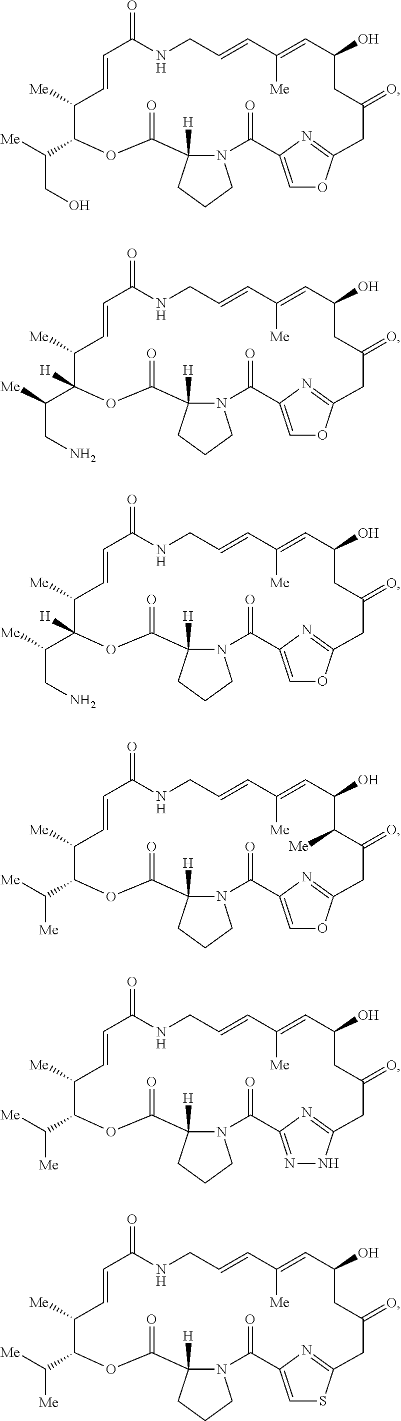

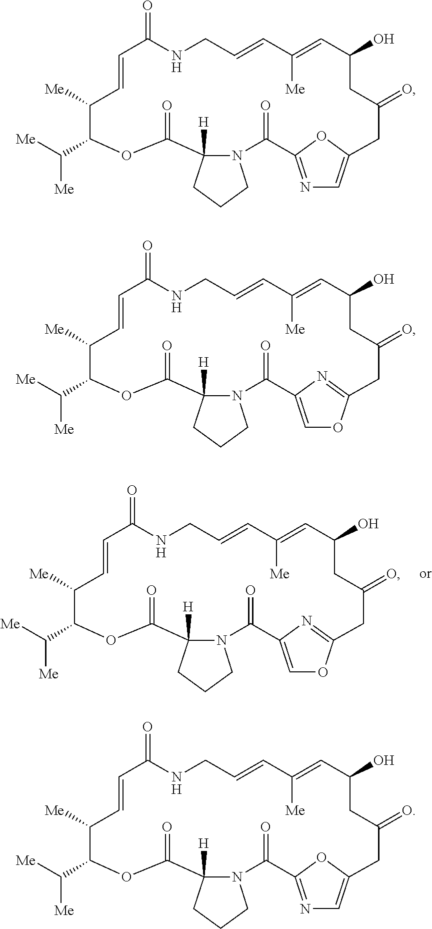 [SMILES: CC1=C\[C@@H](O)CC(=O)CC2=CN=C(O2)C(=O)N2CCC[C@@H]2C(=O)O[C@H](C(C)C)[C@H](C)/C=C/C(=O)NC\C=C\1 ; CC1=C\[C@@H](O)CC(=O)Cc2nc(co2)C(=O)N2CCC[C@@H]2C(=O)O[C@H](C(C)C)[C@H](C)/C=C/C(=O)NC\C=C\1 ; CC1=C\[C@@H](O)CC(=O)Cc2nc(co2)C(=O)N2CCC[C@@H]2C(=O)O[C@H](C(C)C)[C@H](C)/C=C/C(=O)NC\C=C\1 ; CC1=C\[C@@H](O)CC(=O)CC2=CN=C(O2)C(=O)N2CCC[C@@H]2C(=O)O[C@H](C(C)C)[C@H](C)/C=C/C(=O)NC\C=C\1]

In embodiments, compound D has the formula:

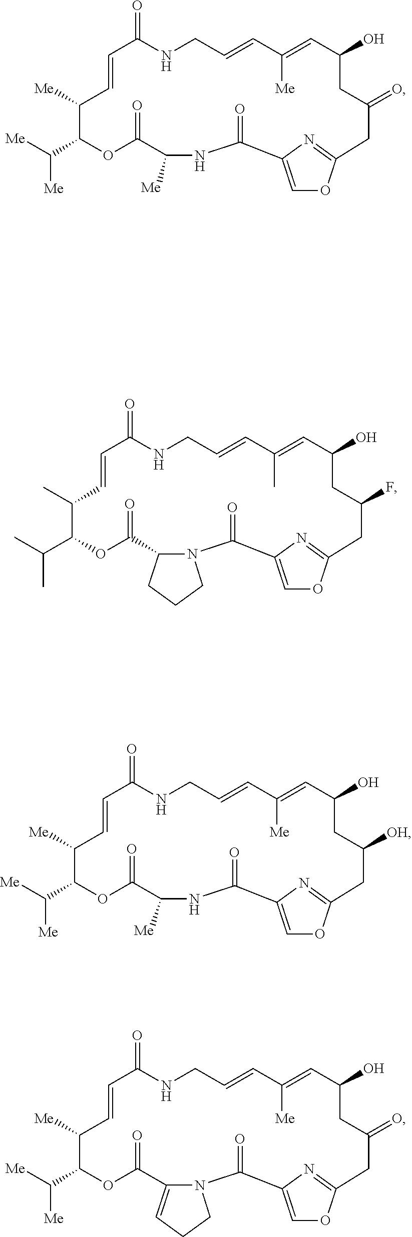 [SMILES: CC1=C\[C@@H](O)CC(=O)Cc2nc(co2)C(=O)N[C@H](C)C(=O)O[C@H](C(C)C)[C@H](C)/C=C/C(=O)NC\C=C\1 ; CC1=C\[C@@H](O)C[C@@H](F)Cc2nc(co2)C(=O)N2CCC[C@@H]2C(=O)O[C@H](C(C)C)[C@H](C)/C=C/C(=O)NC\C=C\1 ; CC1=C\[C@@H](O)C[C@@H](O)Cc2nc(co2)C(=O)N[C@H](C)C(=O)O[C@H](C(C)C)[C@H](C)/C=C/C(=O)NC\C=C\1 ; CC1=C\[C@@H](O)CC(=O)Cc2nc(co2)C(=O)N2CCC=C2C(=O)O[C@H](C(C)C)[C@H](C)/C=C/C(=O)NC\C=C\1]

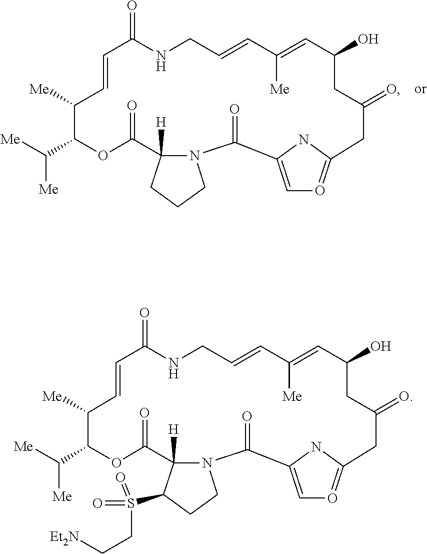

In embodiments, compound D does not have the formula:

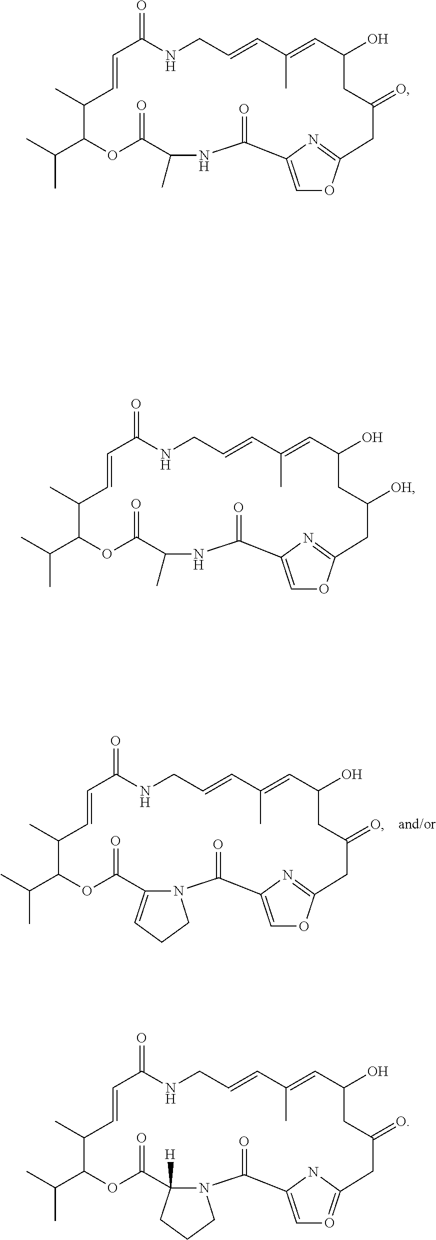

In embodiments, compound D does not have the formula:

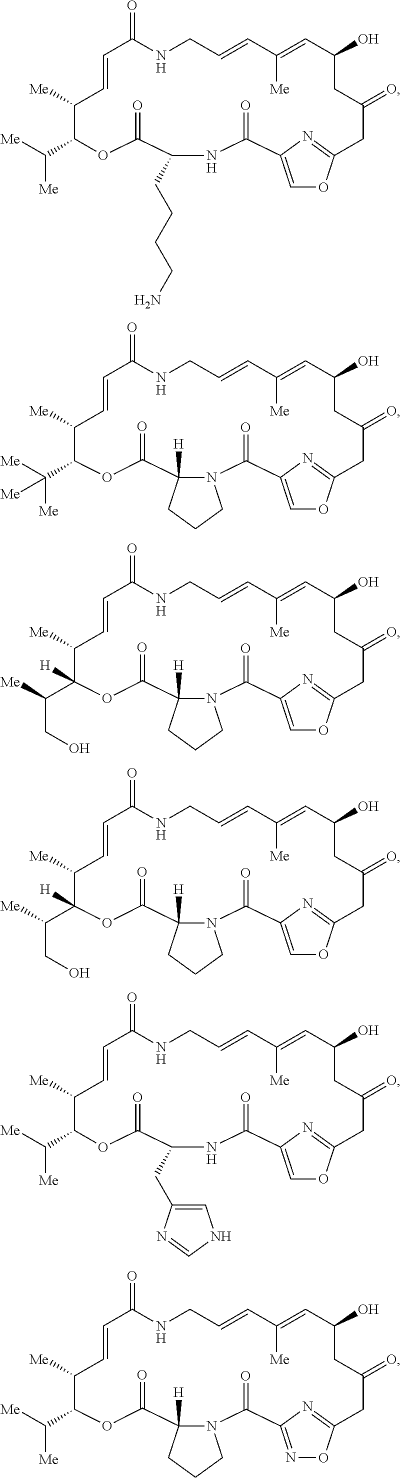

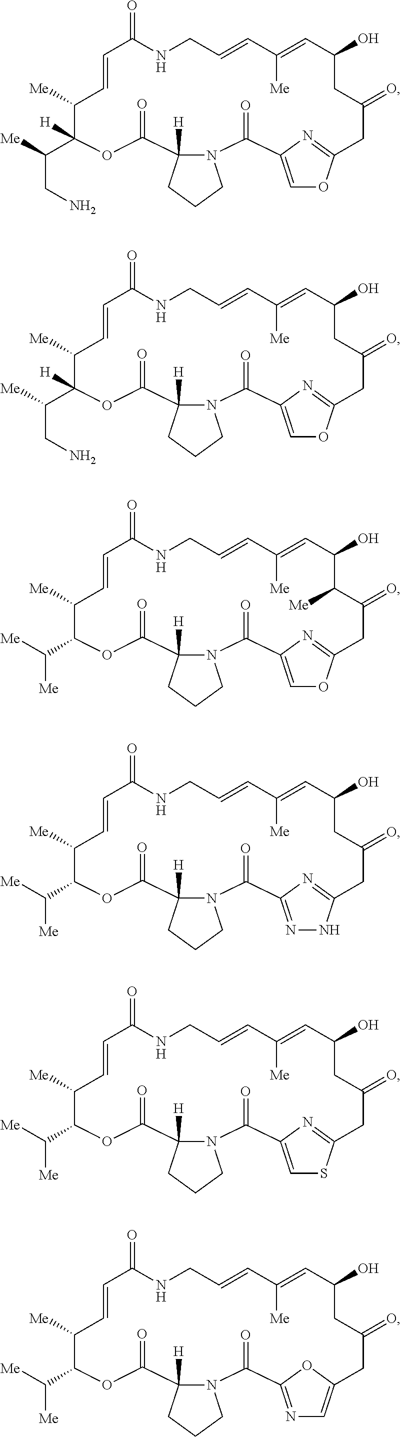

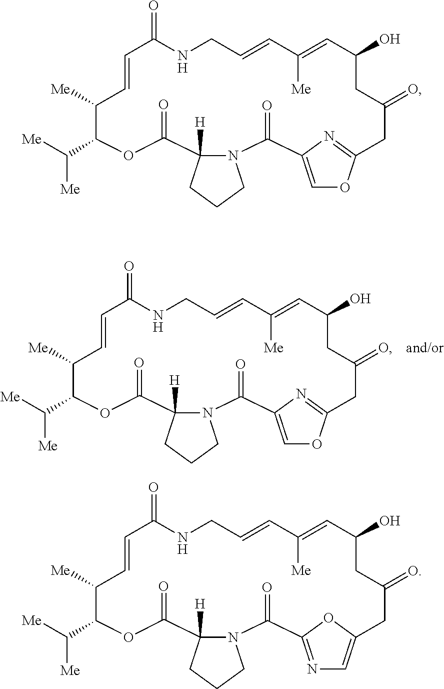

In embodiments, compound D does not have the formula:

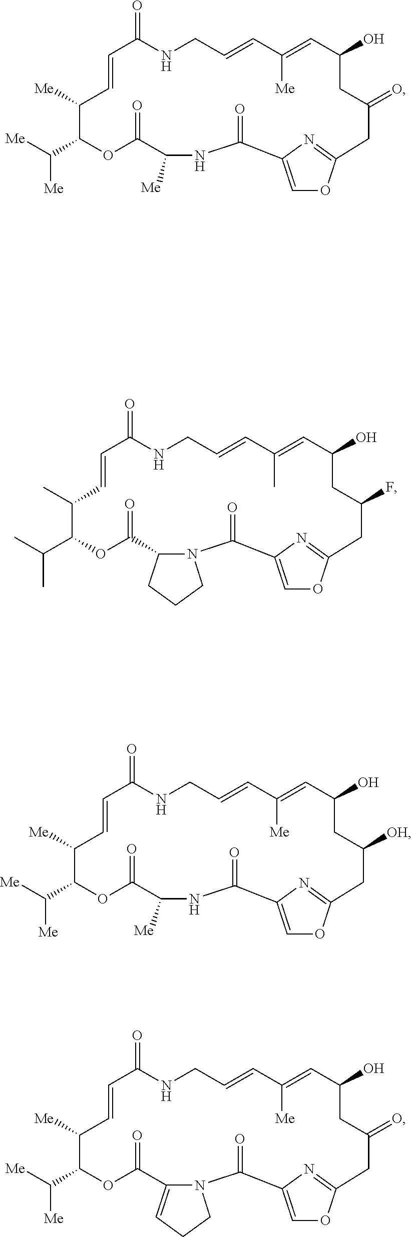 [SMILES: CC1=C\[C@@H](O)CC(=O)Cc2nc(co2)C(=O)N[C@H](C)C(=O)O[C@H](C(C)C)[C@H](C)/C=C/C(=O)NC\C=C\1 ; CC1=C\[C@@H](O)C[C@@H](F)Cc2nc(co2)C(=O)N2CCC[C@@H]2C(=O)O[C@H](C(C)C)[C@H](C)/C=C/C(=O)NC\C=C\1 ; CC1=C\[C@@H](O)C[C@@H](O)Cc2nc(co2)C(=O)N[C@H](C)C(=O)O[C@H](C(C)C)[C@H](C)/C=C/C(=O)NC\C=C\1 ; CC1=C\[C@@H](O)CC(=O)Cc2nc(co2)C(=O)N2CCC=C2C(=O)O[C@H](C(C)C)[C@H](C)/C=C/C(=O)NC\C=C\1]

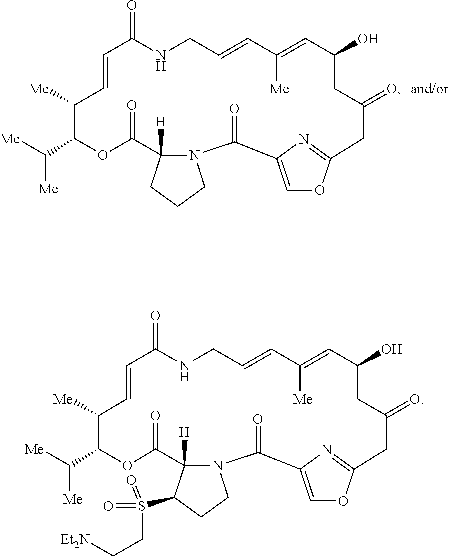

In an aspect is provided a pharmaceutical composition including a compound described herein and a pharmaceutically acceptable excipient.

In embodiments, the compound has the formula:

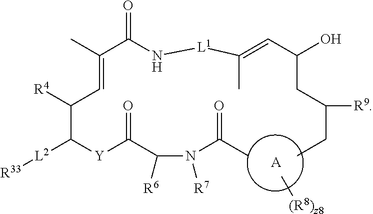

L¹. R⁴, R⁶, R⁷, R⁸, z8, and R⁹ are as described herein, including embodiments. In embodiments, the compound has the formula:

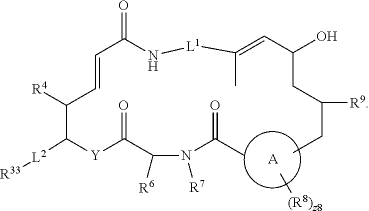

L¹, R⁴, R⁶, R⁷, R⁸, z8, and R⁹ are as described herein, including embodiments. L² is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R³³ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₂, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, -L²- is substituted or unsubstituted C₁-C₈ alkylene, substituted or unsubstituted 2 to 8 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene. In embodiments, -L²- is substituted or unsubstituted C₁-C₈ alkylene, or substituted or unsubstituted 2 to 8 membered heteroalkylene.

In embodiments, -L²- is R³³-substituted or unsubstituted C₁-C₈ alkylene, R³³-substituted or unsubstituted 2 to 8 membered heteroalkylene, R³³-substituted or unsubstituted C₃-C₈ cycloalkylene, R³³-substituted or unsubstituted 3 to 8 membered heterocycloalkylene, R³³-substituted or unsubstituted C₆-C₁₀ arylene, or R³³-substituted or unsubstituted 5 to 10 membered heteroarylene. In embodiments, -L²- is R³³-substituted or unsubstituted C₁-C₈ alkylene, or R³³-substituted or unsubstituted 2 to 8 membered heteroalkylene.

In embodiments, -L²- is

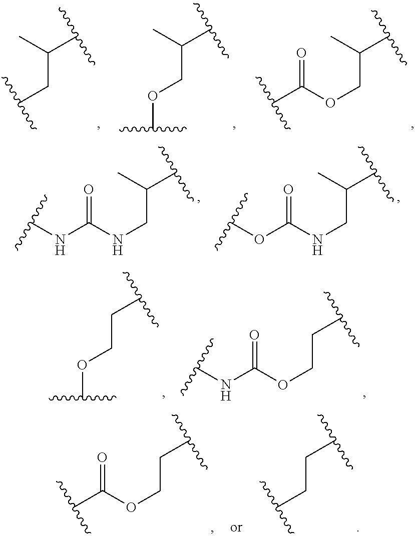

In embodiments, -L²-R³³ is

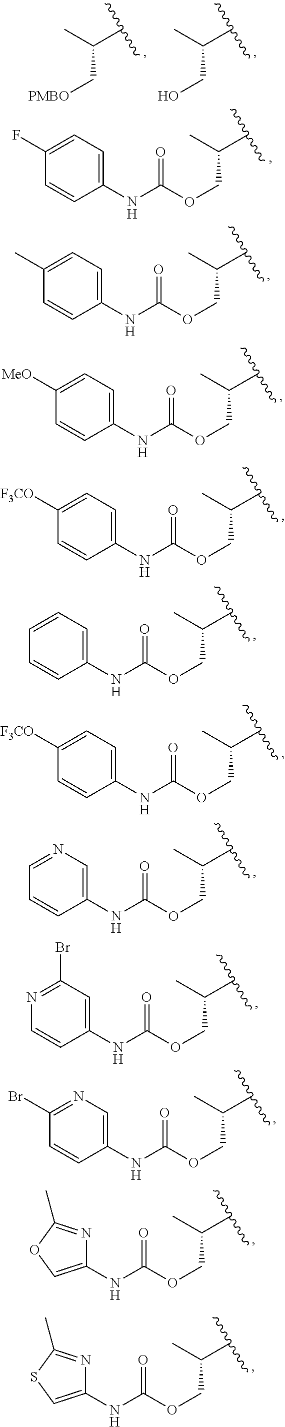

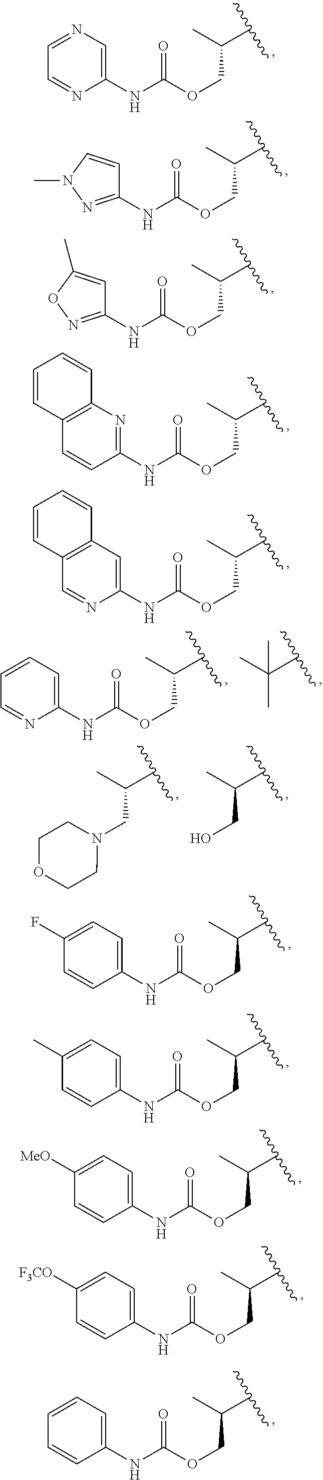

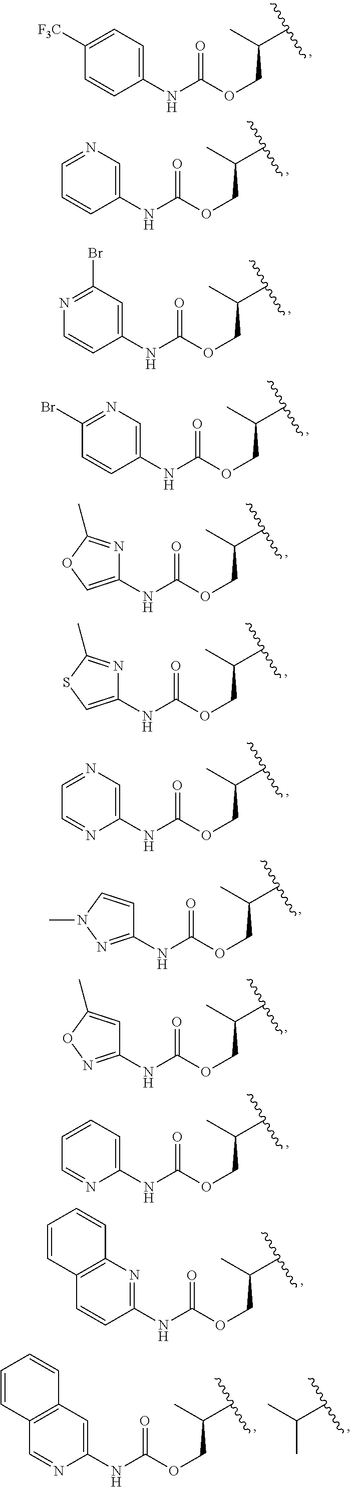

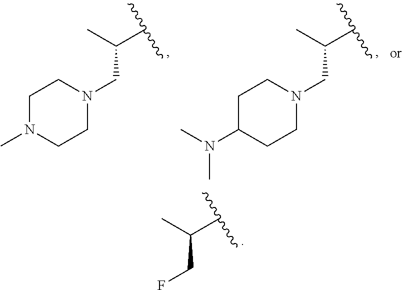

In an aspect is a compound, or a salt thereof, having the formula:

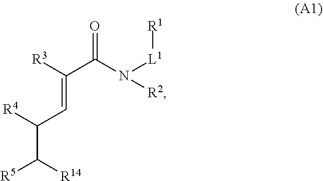

wherein R¹ is bioconjugate reactive moiety or leaving group; Y is —O— or —NH—; L¹ is a bond, substituted or unsubstituted alkylene, or substituted or unsubstituted heteroalkylene; R² is hydrogen or unsubstituted C₁-C₃ alkyl; R³, R⁴, and R⁵ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R¹⁴ is bioconjugate reactive moiety or leaving group.

In an aspect is a compound, or a salt thereof, having the formula:

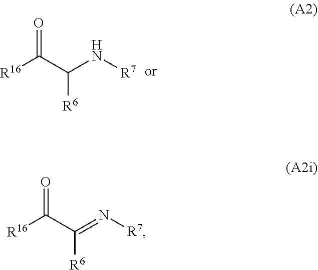

wherein R⁶ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁷ is hydrogen, halogen, —CCl₂, —CBr₃, —CF₃, —CI₃, CHCl₂, —CHBr₂, —CHF₂, —CHI₂, —CH₂Cl, —CH₂Br, —CH₂F, —CH₂I, —CN, —CCl₃, —COOH, —CH₂COOH, —CONH₂, —OH, —SH, —NO₂, —NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R⁶ and R⁷ may optionally be joined to form a substituted or unsubstituted heterocycloalkyl or substituted or unsubstituted heteroaryl; and R¹⁶ is a bioconjugate reactive moiety or leaving group.

In an aspect is a compound, or a salt thereof, having the formula:

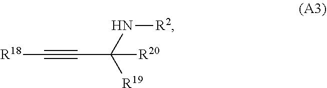

wherein R² is hydrogen or unsubstituted C₁-C₃ alkyl; R¹⁸, R¹⁹, and R²⁰ are independently hydrogen, substituted or unsubstituted alkyl, or substituted or unsubstituted heteroalkyl.

In an aspect is a compound, or a salt thereof, having the formula:

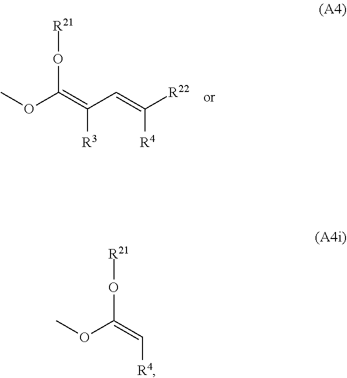

wherein R³ and R⁴ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R²² is independently hydrogen, substituted or unsubstituted alkyl, substituted or unsubstituted heteroalkyl; and R²¹ is hydrogen or a protecting group.

In an aspect is a compound, or a salt thereof, having the formula:

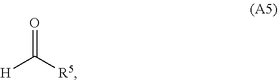

wherein R⁵ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In an aspect is a compound, or a salt thereof, having the formula:

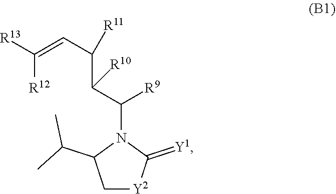

wherein R¹³ is a bioconjugate reactive moiety or leaving group; R⁹, R¹⁰, R¹¹, and R¹² are each independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₂, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R⁹ is hydrogen, oxo, halogen, —CN, —OH, —NH₂, —COOH, —CONH₂, —NO₂, —SH, —SO₃H, —N₃, unsubstituted alkyl, or unsubstituted heteroalkyl; R¹⁰ and R¹² are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R¹¹ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and the symbol Y¹ is ═O or S. The symbol Y² is —O— or —S—.

In an aspect is a compound, or a salt thereof, having the formula:

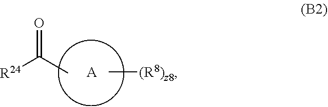

wherein R⁸ is oxo, halogen, —CCl₂, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; Ring A is cycloalkylene, heterocycloalkylene, arylene, or heteroarylene; z8 is an integer from 0 to 10; and R²⁴ is a bioconjugate reactive moiety or leaving group.

In an aspect is a compound, or a salt thereof, having the formula:

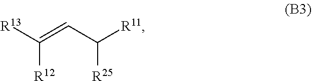

wherein R¹¹ is hydrogen, oxo, halogen, —CCl₂, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹² is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R¹³ and R²⁵ are independently a bioconjugate reactive moiety or leaving group.

In an aspect is a compound, or a salt thereof, having the formula:

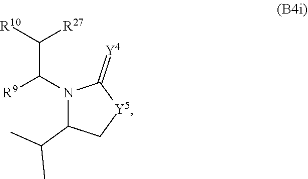

wherein R⁹ and R¹⁰ are each independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₂, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. The symbol Y⁴ is ═O or S. The symbol Y⁵ is —O— or —S—. R²⁷ is a bioconjugate reactive moiety or leaving group. In embodiments, Y⁴ is ═O. In embodiments, Y⁴ is ═S. In embodiments, Y⁵ is —O—. In embodiments, Y⁵ is —S—. In embodiments, R⁹ is hydrogen, oxo, halogen, —CN, —OH, —NH₂, —COOH, —CONH₂, —NO₂, —SH, —SO₃H, —N₃, unsubstituted alkyl, or unsubstituted heteroalkyl; and R¹⁰ is hydrogen or unsubstituted C₁-C₃ alkyl.

In an aspect is a compound, or a salt thereof, having the formula:

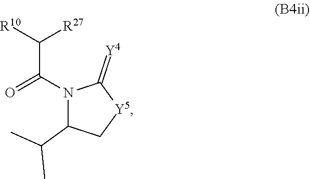

wherein R¹⁰ is as described herein; the symbol Y⁴ is ═O or ═S. The symbol Y⁵ is —O— or —S—. R²⁷ is a bioconjugate reactive moiety or leaving group. In embodiments, Y⁴ is ═O. In embodiments, Y⁴ is ═S. In embodiments, Y⁵ is —O—. In embodiments, Y⁵ is —S—.

In embodiments, the compound has the formula:

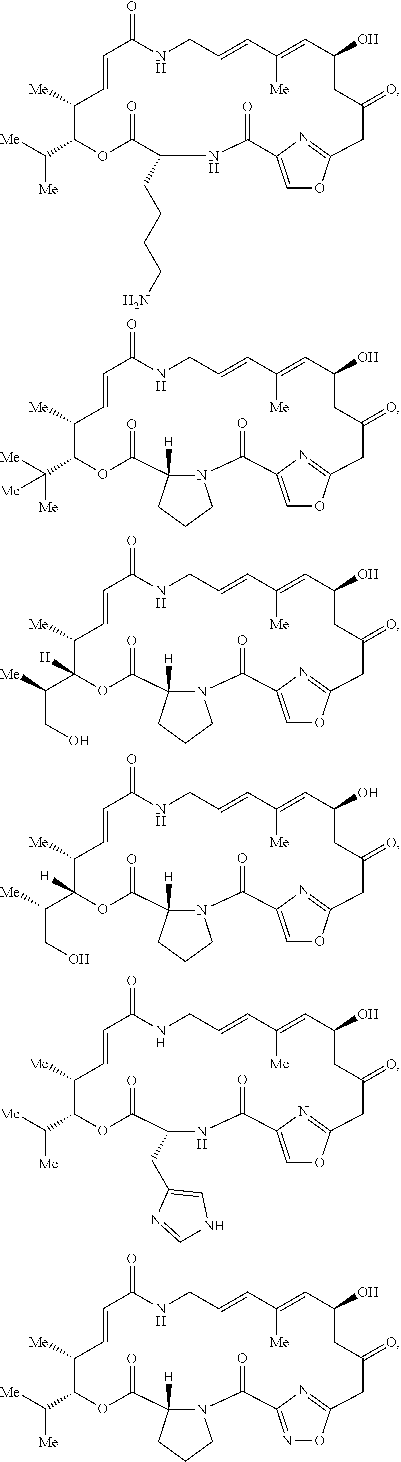

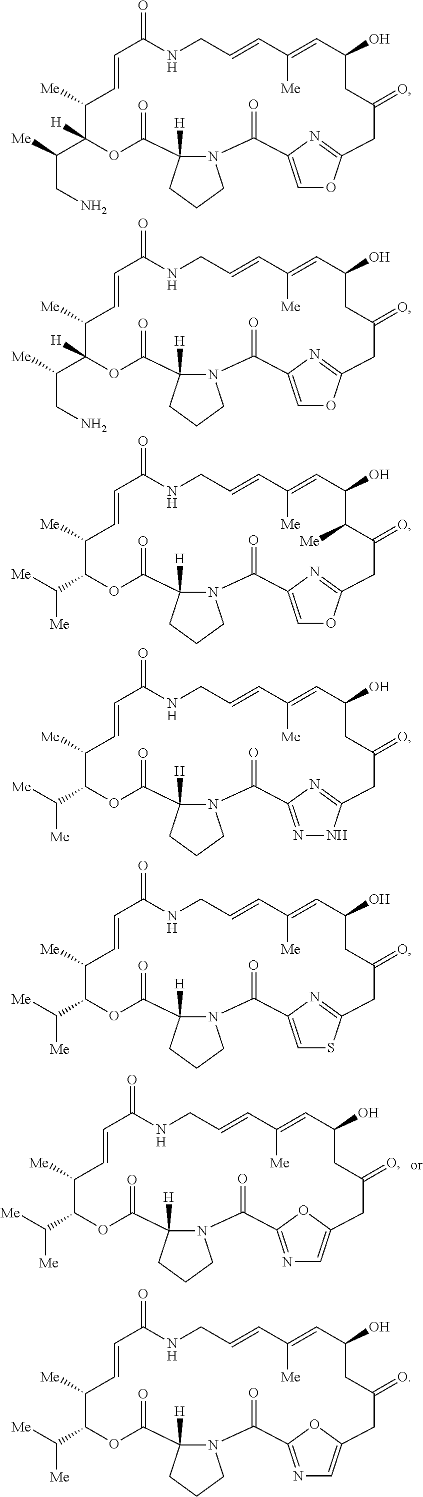

In embodiments, the compound has the formula:

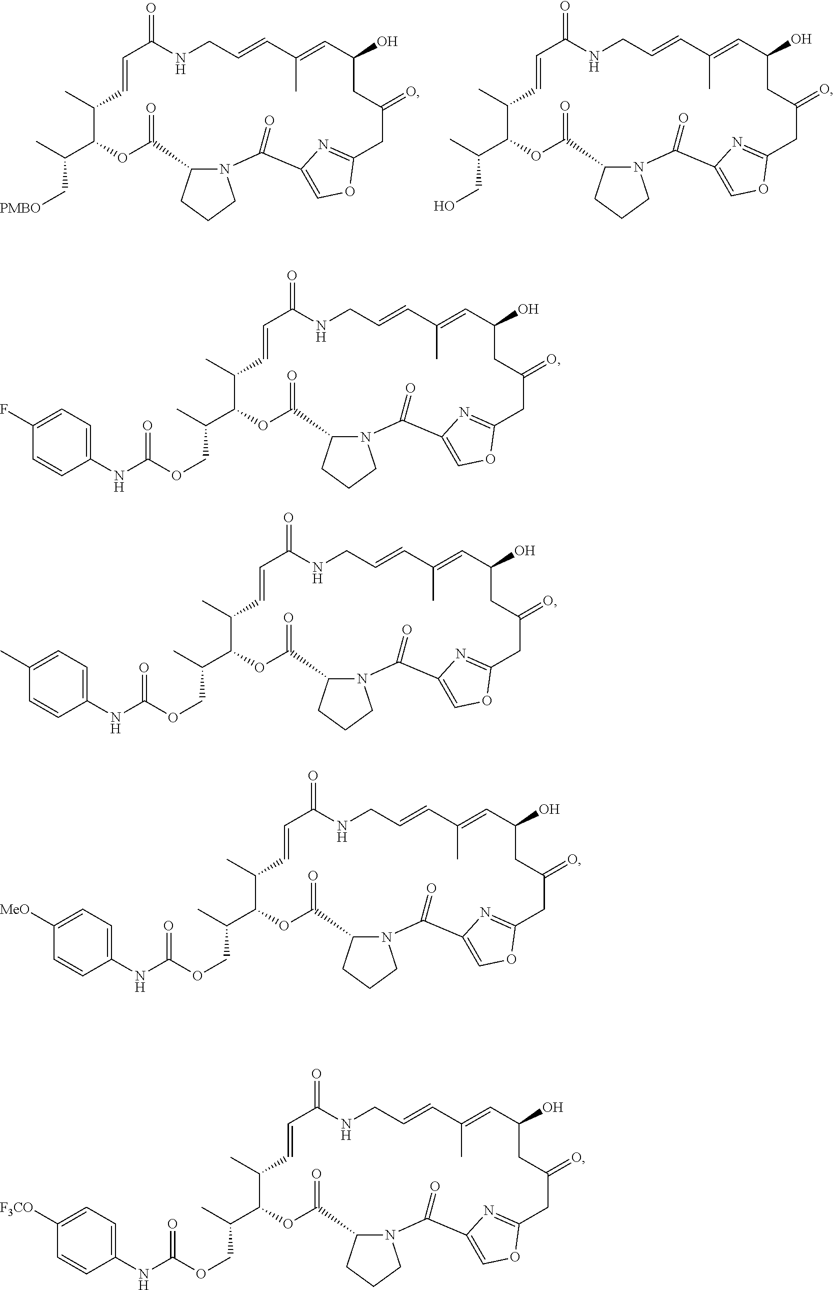

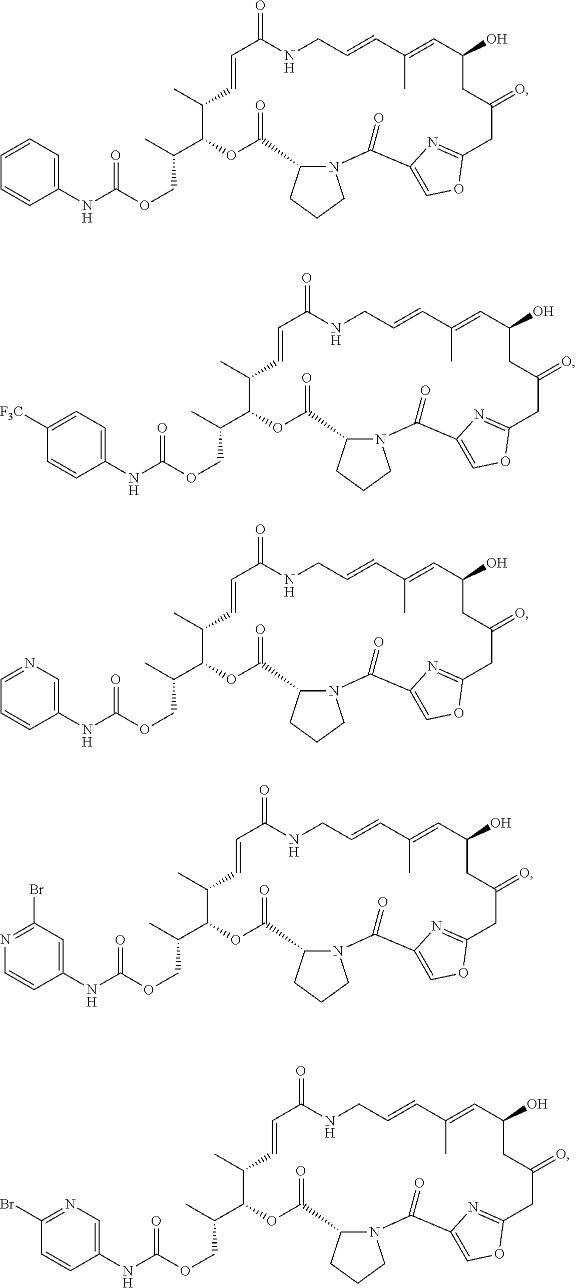

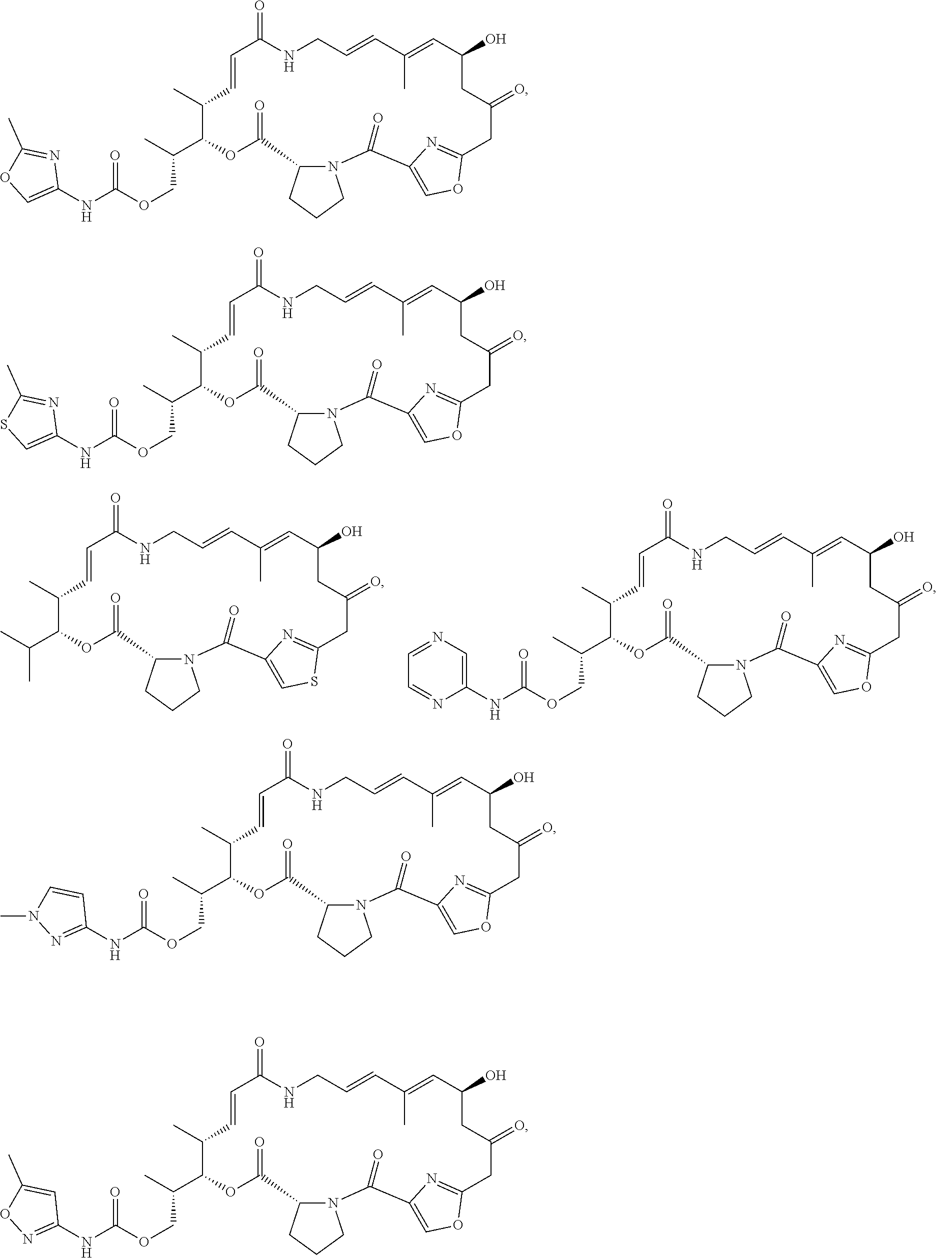

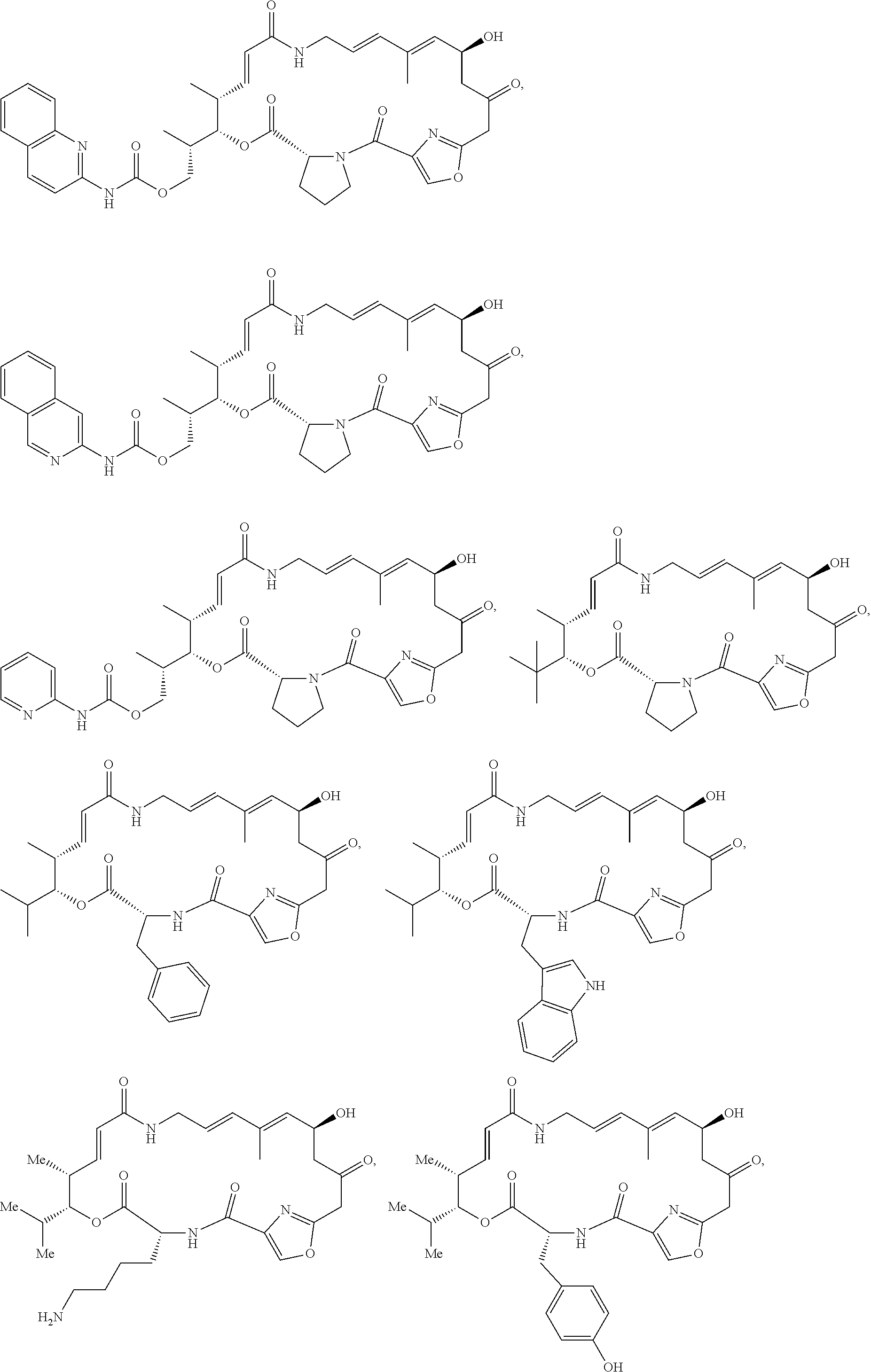

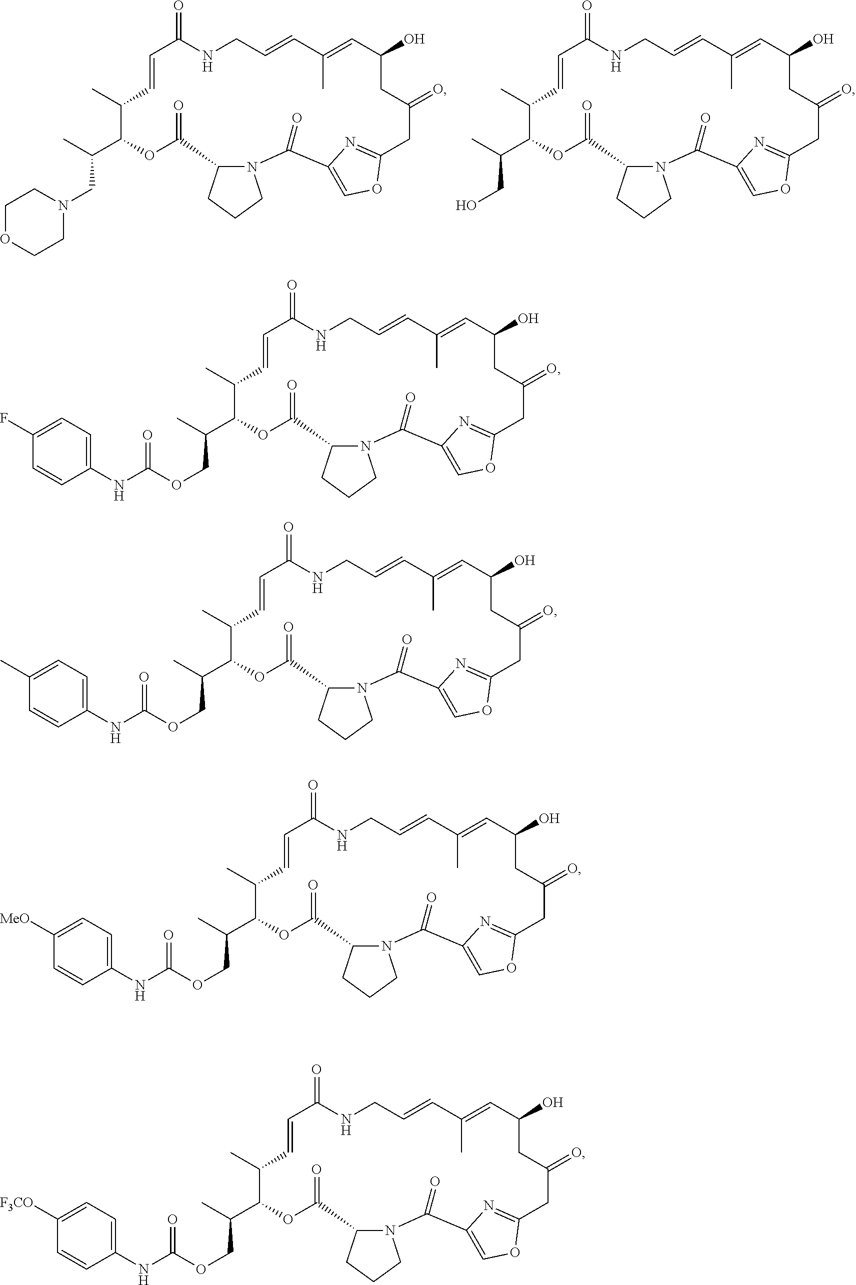

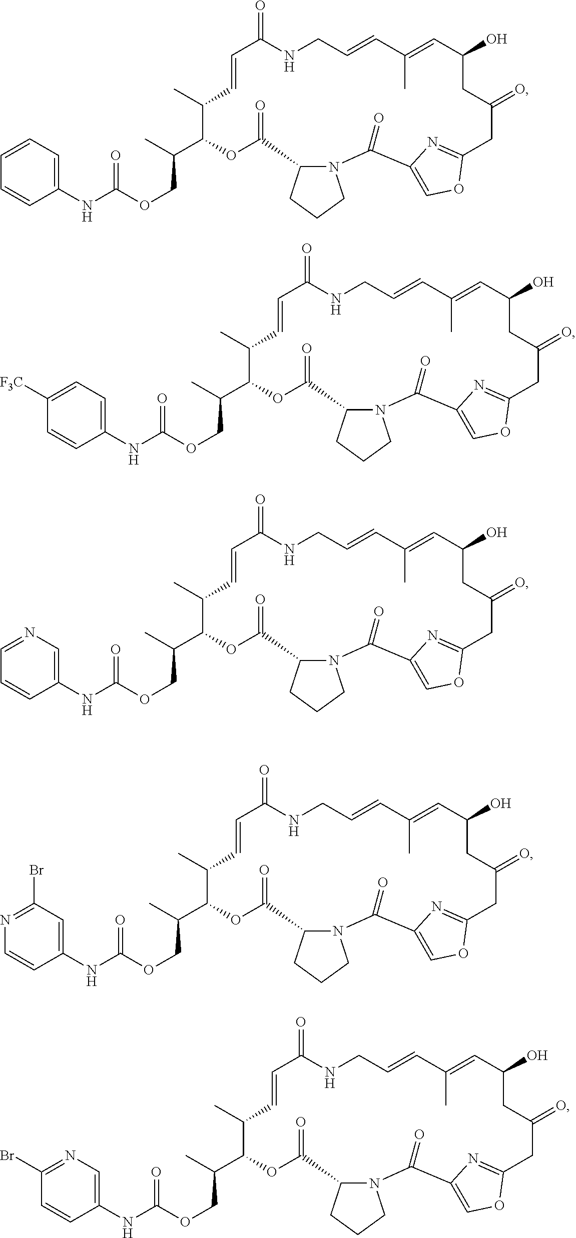

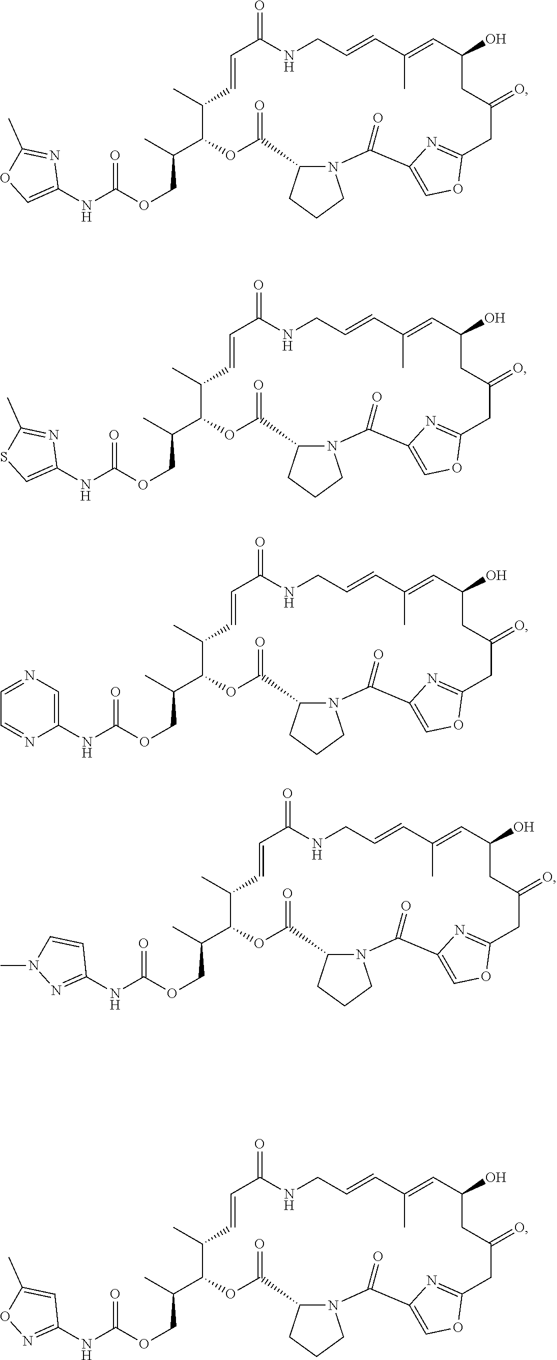

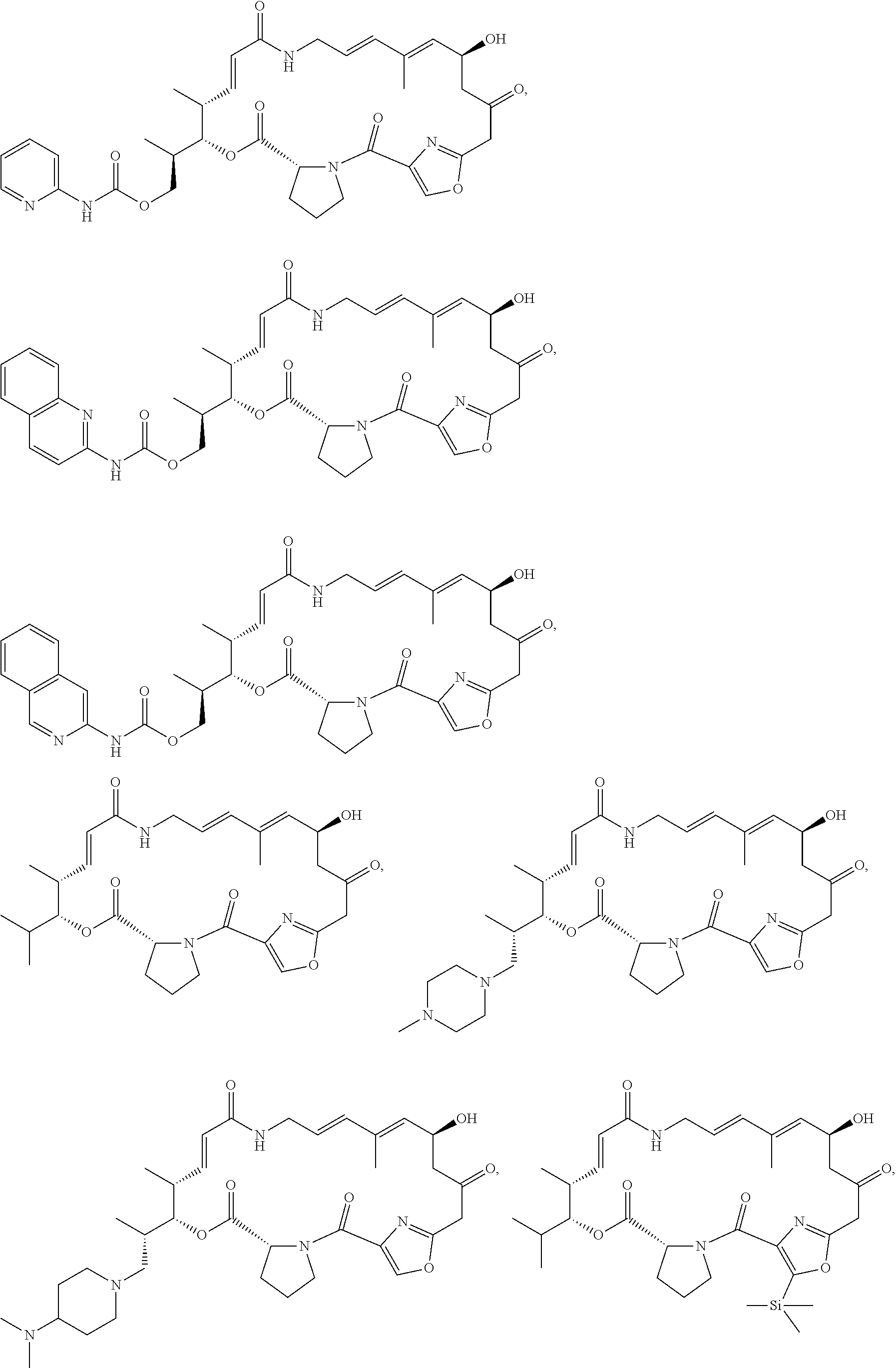

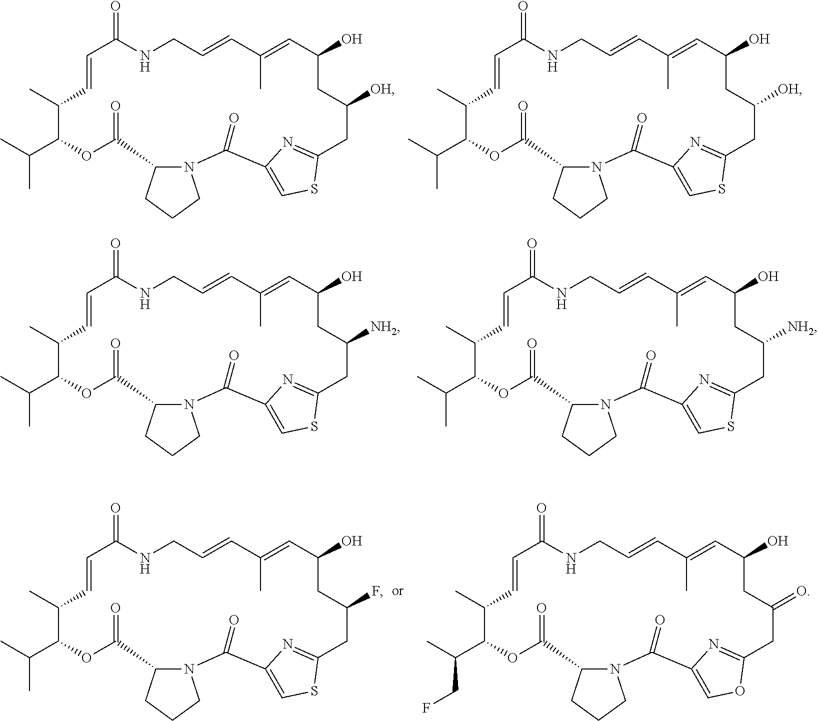

In embodiments, the compound has the formula:

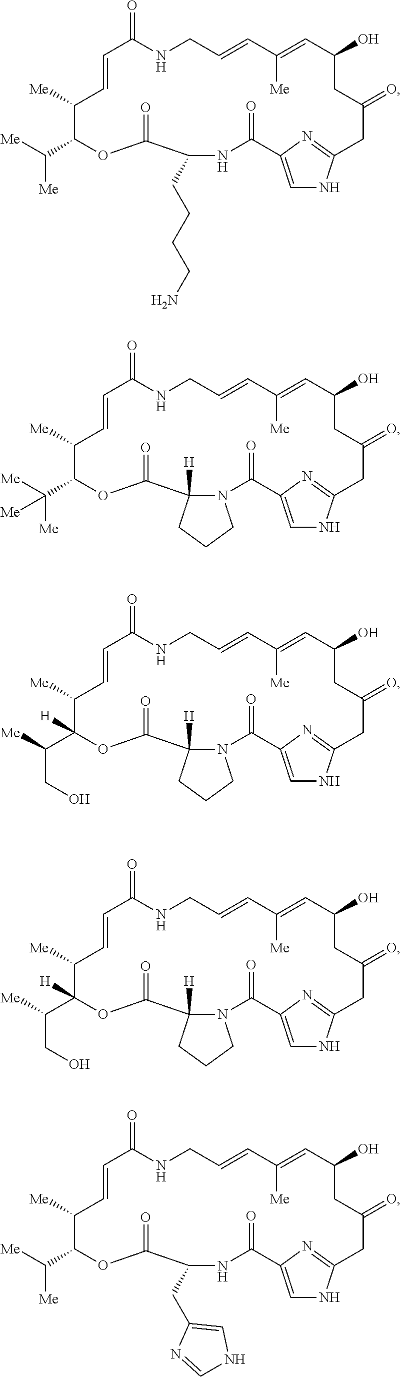

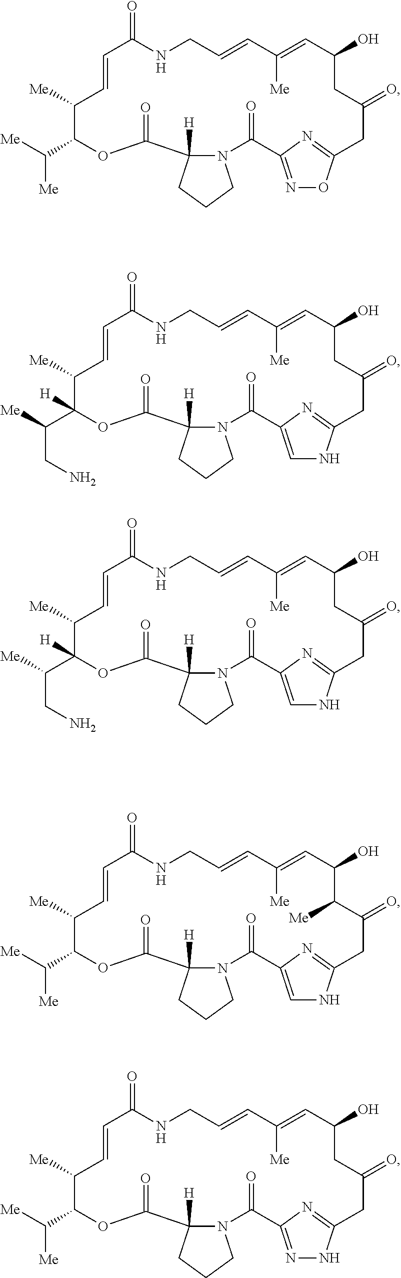

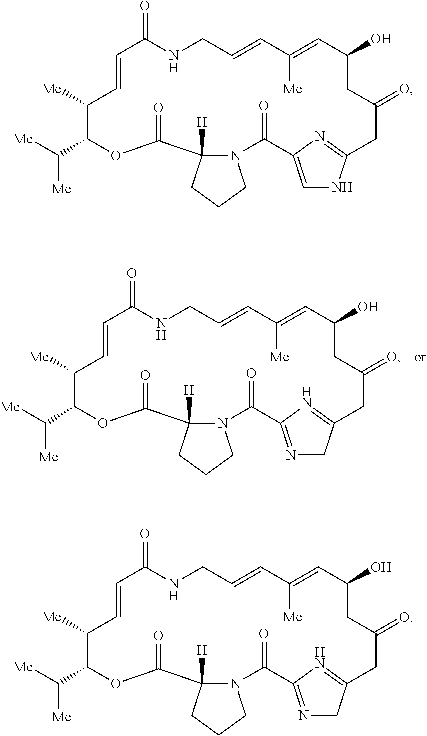

In embodiments, the leaving group (e.g., R¹, R¹³, R¹⁴, R¹⁶, R²⁴, R²³, or R²⁷) is independently hydrogen, hydroxide, organotin moiety (e.g., organotin heteroalkyl), halogen (e.g., Br), perfluoroalkylsulfonates (e.g. triflate), tosylate, mesylate, water, alcohol, nitrate, phosphate, thioether, amine, ammonia, fluoride, carboxylate, phenoxide, boronic acid, boronate ester, or an alkoxide. In embodiments, the leaving group is a substituent group.

In embodiments, the atom or chemical moieties to which R¹⁴ and R¹⁶ are attached (e.g., adjacent atoms) form a complementary reactive group pair (e.g., bioconjugate reactive moieties). For example, R¹⁴ and the carbonyl moiety to which R¹⁶ is attached may be complementary reactive group pairs (e.g., complementary reactive groups react to form a bioconjugate reactive linker (i.e. a covalent bond)).

In embodiments, the atom or chemical moieties to which R²⁴ and R⁷ are attached (e.g., adjacent atoms) form a complementary reactive group pair (e.g., bioconjugate reactive moieties). For example, the nitrogen to which R⁷ is attached and the carbonyl moiety to which R²⁴ is attached may be complementary reactive group pairs (e.g., complementary reactive groups react to form a bioconjugate reactive linker (i.e. a covalent bond)).

In embodiments, the atom or chemical moieties to which R²⁵ and R²⁷ are attached (e.g., adjacent atoms) form a complementary reactive group pair (e.g., bioconjugate reactive moieties). For example, the methyl moiety to which R²⁵ is attached and the methyl moiety to which R²⁷ is attached may be complementary reactive group pairs (e.g., complementary reactive groups react to form a bioconjugate reactive linker (i.e. a covalent bond)).

In embodiments, R¹⁴ is a leaving group. In embodiments, R¹⁴ is hydrogen, —OH, or —NH₂. In embodiments, R¹⁴ is hydrogen. In embodiments, R¹⁴ is —OH. In embodiments, R¹⁴ is —NH₂. In embodiments, R¹⁴ and R¹⁶ leave the atom to which they were attached following a reaction (e.g., hydrolysis or condensation reaction). In embodiments, R¹⁴ and R¹⁶ are bioconjugate reactive moieties. In embodiments, R¹⁴ and R¹⁶ are allowed to contact such that R¹⁴ and R¹⁶ are sufficiently proximal to react, interact or physically touch. In embodiments, R¹⁴ and R¹⁶ are leaving groups following a reaction (e.g., a cross coupling reaction). In embodiments, R¹⁶ is a leaving group. In embodiments, R¹⁶ is hydrogen, —OH, or —NH₂. In embodiments, R¹⁶ is hydrogen. In embodiments, R¹⁶ is —OH. In embodiments, R¹⁶ is —NH₂.

In embodiments, R²⁴ is a leaving group. In embodiments, R²⁴ is hydrogen, —OH, or —NH₂. In embodiments, R²⁴ is hydrogen. In embodiments, R²⁴ is —OH. In embodiments, R²⁴ is —NH₂.

In embodiments, R²⁵ is a leaving group. In embodiments, R²⁵ is hydrogen, —OH, or —NH₂. In embodiments, R²⁵ is hydrogen. In embodiments, R²⁵ is —OH. In embodiments, R²⁵ is —NH₂.

In embodiments, R²⁷ is a leaving group. In embodiments, R²⁷ is hydrogen, —OH, or —NH₂. In embodiments, R²⁷ is hydrogen. In embodiments, R²⁷ is —OH. In embodiments, R²⁷ is —NH₂.

In embodiments, R²¹ is hydrogen or a protecting group. In embodiments, R²¹ is hydrogen. In embodiments, R²¹ is a protecting group. In embodiments, R²¹ is tert-butyldimethylsilyl ether (TBS).

In embodiments, the compound is a compound described herein, including embodiments, figures, table, claim, or within the examples (e.g., in a scheme).

In embodiments, the compound is

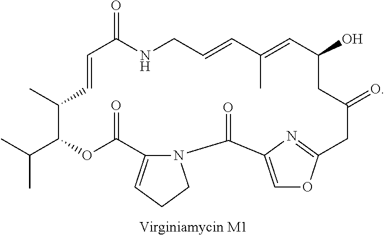 [SMILES: CC1=C\[C@@H](O)CC(=O)Cc2nc(co2)C(=O)N2CCC=C2C(=O)O[C@H](C(C)C)[C@H](C)/C=C/C(=O)NC\C=C\1]

In embodiments, the compound is

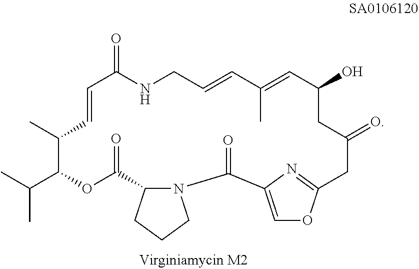 [SMILES: CC1=C\[C@@H](O)CC(=O)Cc2nc(co2)C(=O)N2CCC[C@@H]2C(=O)O[C@H](C(C)C)[C@H](C)/C=C/C(=O)NC\C=C\1]

In embodiments, the compound is

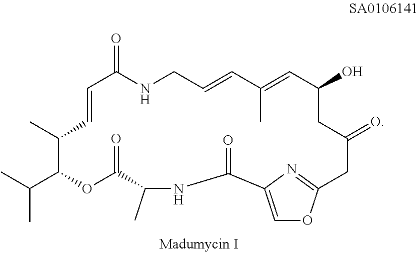 [SMILES: CC1=C\[C@@H](O)CC(=O)Cc2nc(co2)C(=O)N[C@H](C)C(=O)O[C@H](C(C)C)[C@H](C)/C=C/C(=O)NC\C=C\1]

In embodiments, the compound is

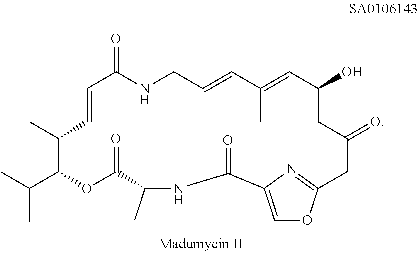 [SMILES: CC1=C\[C@@H](O)CC(=O)Cc2nc(co2)C(=O)N[C@H](C)C(=O)O[C@H](C(C)C)[C@H](C)/C=C/C(=O)NC\C=C\1]

In embodiments, the compound is

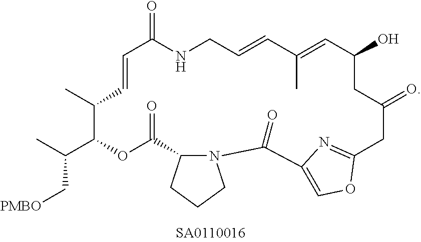

In embodiments, the compound is

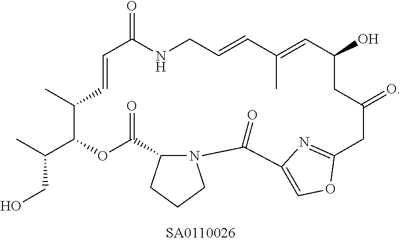 [SMILES: CC1=C\[C@@H](O)CC(=O)Cc2nc(co2)C(=O)N2CCC[C@@H]2C(=O)O[C@H]([C@@H](C)CO)[C@H](C)/C=C/C(=O)NC\C=C\1]

In embodiments, the compound is

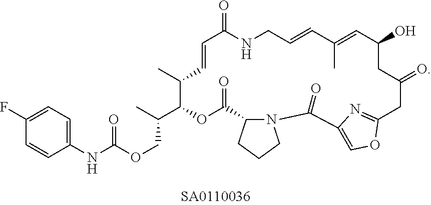 [SMILES: CC1=C\[C@@H](O)CC(=O)Cc2nc(co2)C(=O)N2CCC[C@@H]2C(=O)O[C@H]([C@@H](C)COC(=O)Nc2ccc(F)cc2)[C@H](C)/C=C/C(=O)NC\C=C\1]

In embodiments, the compound is

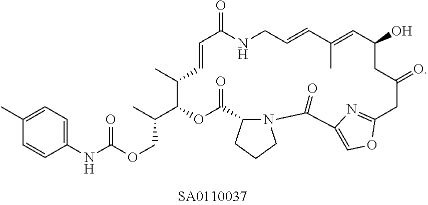 [SMILES: CC1=C\[C@@H](O)CC(=O)Cc2nc(co2)C(=O)N2CCC[C@@H]2C(=O)O[C@H]([C@@H](C)COC(=O)Nc2ccc(C)cc2)[C@H](C)/C=C/C(=O)NC\C=C\1]

In embodiments, the compound is

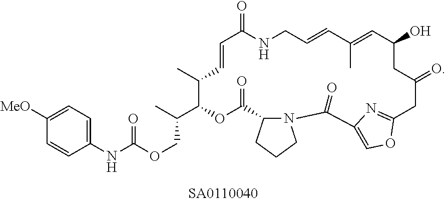 [SMILES: COc1ccc(NC(=O)OC[C@H](C)[C@H]2OC(=O)[C@H]3CCCN3C(=O)c3coc(n3)CC(=O)C[C@H](O)/C=C(C)/C=C/CNC(=O)/C=C/[C@H]2C)cc1]

In embodiments, the compound is

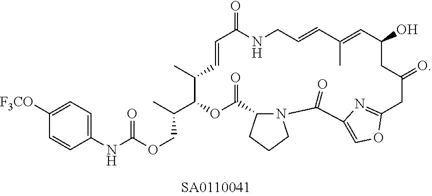 [SMILES: CC1=C\[C@@H](O)CC(=O)Cc2nc(co2)C(=O)N2CCC[C@@H]2C(=O)O[C@H]([C@@H](C)COC(=O)Nc2ccc(OC(F)(F)F)cc2)[C@H](C)/C=C/C(=O)NC\C=C\1]

In embodiments, the compound is

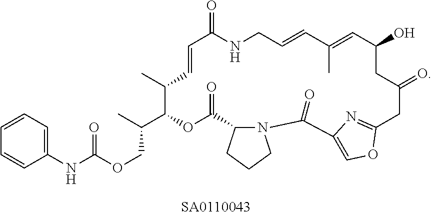 [SMILES: CC1=C\[C@@H](O)CC(=O)Cc2nc(co2)C(=O)N2CCC[C@@H]2C(=O)O[C@H]([C@@H](C)COC(=O)Nc2ccccc2)[C@H](C)/C=C/C(=O)NC\C=C\1]

In embodiments, the compound is

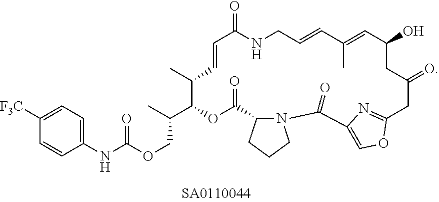 [SMILES: CC1=C\[C@@H](O)CC(=O)Cc2nc(co2)C(=O)N2CCC[C@@H]2C(=O)O[C@H]([C@@H](C)COC(=O)Nc2ccc(C(F)(F)F)cc2)[C@H](C)/C=C/C(=O)NC\C=C\1]

In embodiments, the compound is

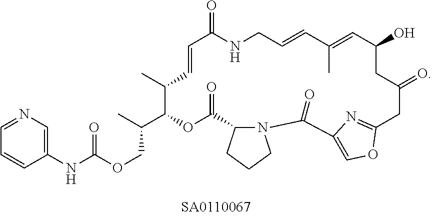 [SMILES: CC1=C\[C@@H](O)CC(=O)Cc2nc(co2)C(=O)N2CCC[C@@H]2C(=O)O[C@H]([C@@H](C)COC(=O)Nc2cccnc2)[C@H](C)/C=C/C(=O)NC\C=C\1]

In embodiments, the compound is

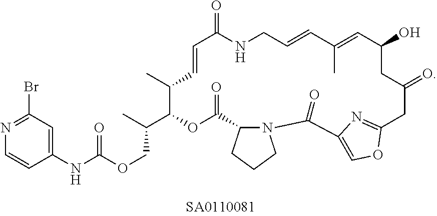 [SMILES: CC1=C\[C@@H](O)CC(=O)Cc2nc(co2)C(=O)N2CCC[C@@H]2C(=O)O[C@H]([C@@H](C)COC(=O)Nc2ccnc(Br)c2)[C@H](C)/C=C/C(=O)NC\C=C\1]

In embodiments, the compound is

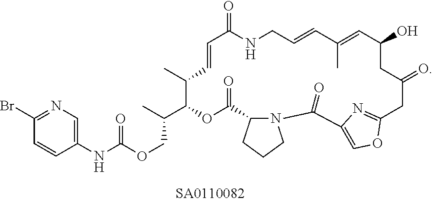 [SMILES: CC1=C\[C@@H](O)CC(=O)Cc2nc(co2)C(=O)N2CCC[C@@H]2C(=O)O[C@H]([C@@H](C)COC(=O)Nc2ccc(Br)nc2)[C@H](C)/C=C/C(=O)NC\C=C\1]

In embodiments, the compound is

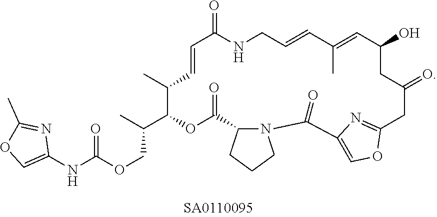 [SMILES: CC1=C\[C@@H](O)CC(=O)Cc2nc(co2)C(=O)N2CCC[C@@H]2C(=O)O[C@H]([C@@H](C)COC(=O)Nc2coc(C)n2)[C@H](C)/C=C/C(=O)NC\C=C\1]

In embodiments, the compound is

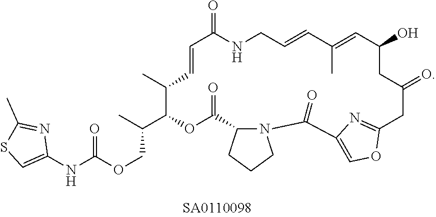 [SMILES: CC1=C\[C@@H](O)CC(=O)Cc2nc(co2)C(=O)N2CCC[C@@H]2C(=O)O[C@H]([C@@H](C)COC(=O)Nc2csc(C)n2)[C@H](C)/C=C/C(=O)NC\C=C\1]

In embodiments, the compound is

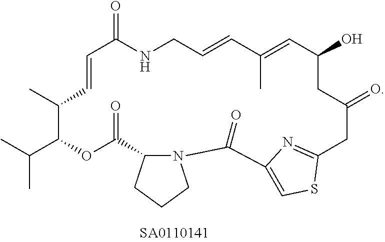 [SMILES: CC1=C\[C@@H](O)CC(=O)Cc2nc(cs2)C(=O)N2CCC[C@@H]2C(=O)O[C@H](C(C)C)[C@H](C)/C=C/C(=O)NC\C=C\1]

In embodiments, the compound is

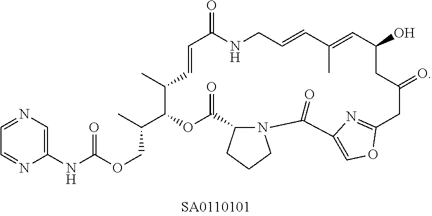 [SMILES: CC1=C\[C@@H](O)CC(=O)Cc2nc(co2)C(=O)N2CCC[C@@H]2C(=O)O[C@H]([C@@H](C)COC(=O)Nc2cnccn2)[C@H](C)/C=C/C(=O)NC\C=C\1]

In embodiments, the compound is

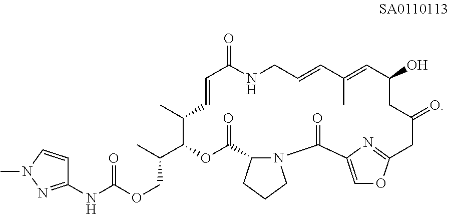 [SMILES: CC1=C\[C@@H](O)CC(=O)Cc2nc(co2)C(=O)N2CCC[C@@H]2C(=O)O[C@H]([C@@H](C)COC(=O)Nc2ccn(C)n2)[C@H](C)/C=C/C(=O)NC\C=C\1]

In embodiments, the compound is

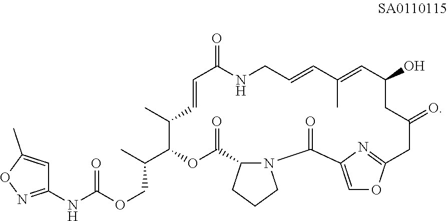 [SMILES: CC1=C\[C@@H](O)CC(=O)Cc2nc(co2)C(=O)N2CCC[C@@H]2C(=O)O[C@H]([C@@H](C)COC(=O)Nc2cc(C)on2)[C@H](C)/C=C/C(=O)NC\C=C\1]

In embodiments, the compound is

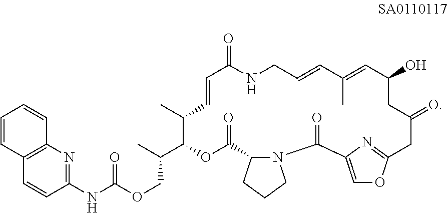 [SMILES: CC1=C\[C@@H](O)CC(=O)Cc2nc(co2)C(=O)N2CCC[C@@H]2C(=O)O[C@H]([C@@H](C)COC(=O)Nc2ccc3ccccc3n2)[C@H](C)/C=C/C(=O)NC\C=C\1]

In embodiments, the compound is

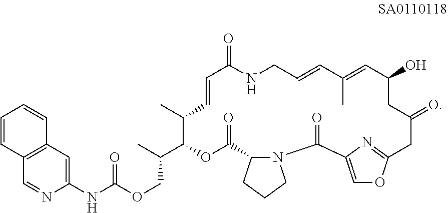 [SMILES: CC1=C\[C@@H](O)CC(=O)Cc2nc(co2)C(=O)N2CCC[C@@H]2C(=O)O[C@H]([C@@H](C)COC(=O)Nc2cc3ccccc3cn2)[C@H](C)/C=C/C(=O)NC\C=C\1]

In embodiments, the compound is

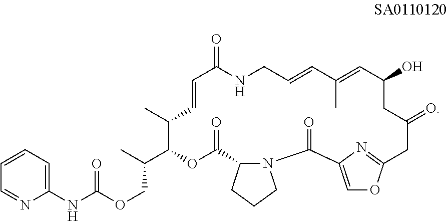 [SMILES: CC1=C\[C@@H](O)CC(=O)Cc2nc(co2)C(=O)N2CCC[C@@H]2C(=O)O[C@H]([C@@H](C)COC(=O)Nc2ccccn2)[C@H](C)/C=C/C(=O)NC\C=C\1]

In embodiments, the compound is

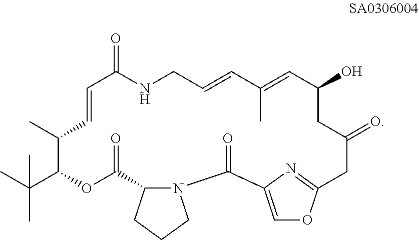 [SMILES: CC1=C\[C@@H](O)CC(=O)Cc2nc(co2)C(=O)N2CCC[C@@H]2C(=O)O[C@H](C(C)(C)C)[C@H](C)/C=C/C(=O)NC\C=C\1]

In embodiments, the compound is

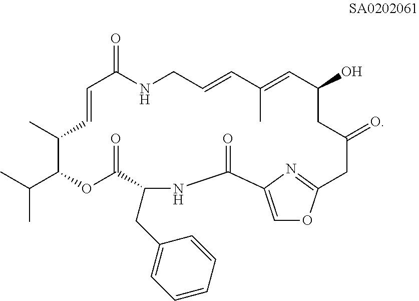 [SMILES: CC1=C\[C@@H](O)CC(=O)Cc2nc(co2)C(=O)N[C@H](Cc2ccccc2)C(=O)O[C@H](C(C)C)[C@H](C)/C=C/C(=O)NC\C=C\1]

In embodiments, the compound is

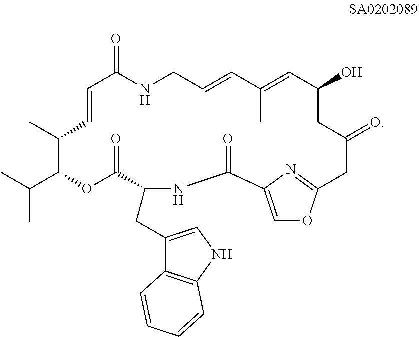 [SMILES: CC1=C\[C@@H](O)CC(=O)Cc2nc(co2)C(=O)N[C@H](Cc2c[nH]c3ccccc23)C(=O)O[C@H](C(C)C)[C@H](C)/C=C/C(=O)NC\C=C\1]

In embodiments, the compound is

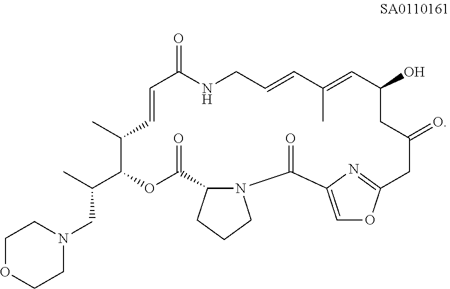 [SMILES: CC1=C\[C@@H](O)CC(=O)Cc2nc(co2)C(=O)N2CCC[C@@H]2C(=O)O[C@H]([C@@H](C)CN2CCOCC2)[C@H](C)/C=C/C(=O)NC\C=C\1]

In embodiments, the compound is

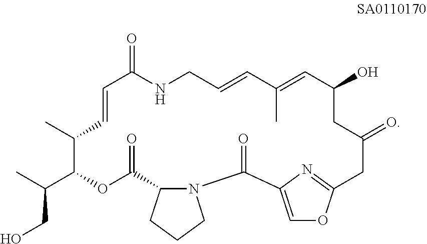 [SMILES: CC1=C\[C@@H](O)CC(=O)Cc2nc(co2)C(=O)N2CCC[C@@H]2C(=O)O[C@H]([C@H](C)CO)[C@H](C)/C=C/C(=O)NC\C=C\1]

In embodiments, the compound is

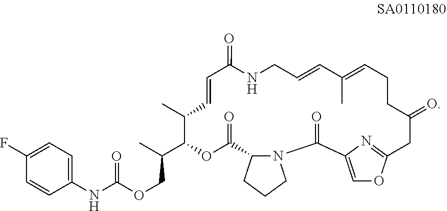 [SMILES: CC1=C\CCC(=O)Cc2nc(co2)C(=O)N2CCC[C@@H]2C(=O)O[C@H]([C@H](C)COC(=O)Nc2ccc(F)cc2)[C@H](C)/C=C/C(=O)NC\C=C\1]

In embodiments, the compound is

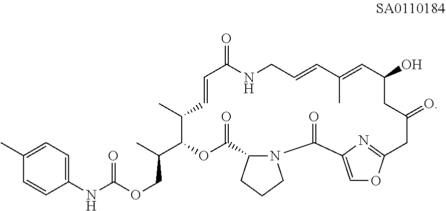 [SMILES: CC1=C\[C@@H](O)CC(=O)Cc2nc(co2)C(=O)N2CCC[C@@H]2C(=O)O[C@H]([C@H](C)COC(=O)Nc2ccc(C)cc2)[C@H](C)/C=C/C(=O)NC\C=C\1]

In embodiments, the compound is

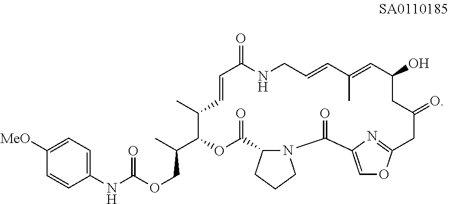 [SMILES: COc1ccc(NC(=O)OC[C@@H](C)[C@H]2OC(=O)[C@H]3CCCN3C(=O)c3coc(n3)CC(=O)C[C@H](O)/C=C(C)/C=C/CNC(=O)/C=C/[C@H]2C)cc1]

In embodiments, the compound is

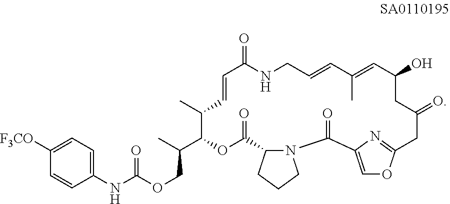 [SMILES: CC1=C\[C@@H](O)CC(=O)Cc2nc(co2)C(=O)N2CCC[C@@H]2C(=O)O[C@H]([C@H](C)COC(=O)Nc2ccc(OC(F)(F)F)cc2)[C@H](C)/C=C/C(=O)NC\C=C\1]

In embodiments, the compound is

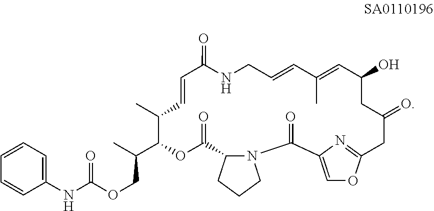 [SMILES: CC1=C\[C@@H](O)CC(=O)Cc2nc(co2)C(=O)N2CCC[C@@H]2C(=O)O[C@H]([C@H](C)COC(=O)Nc2ccccc2)[C@H](C)/C=C/C(=O)NC\C=C\1]

In embodiments, the compound is

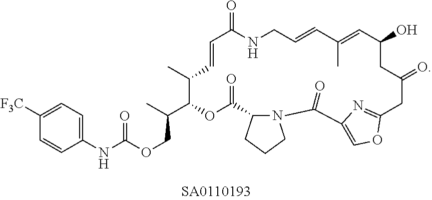 [SMILES: CC1=C\[C@@H](O)CC(=O)Cc2nc(co2)C(=O)N2CCC[C@@H]2C(=O)O[C@H]([C@H](C)COC(=O)Nc2ccc(C(F)(F)F)cc2)[C@H](C)/C=C/C(=O)NC\C=C\1]

In embodiments, the compound is

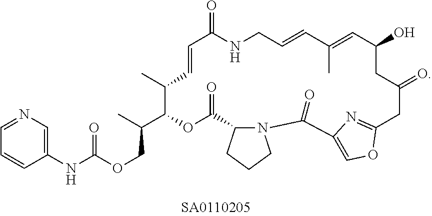 [SMILES: CC1=C\[C@@H](O)CC(=O)Cc2nc(co2)C(=O)N2CCC[C@@H]2C(=O)O[C@H]([C@H](C)COC(=O)Nc2cccnc2)[C@H](C)/C=C/C(=O)NC\C=C\1]

In embodiments, the compound is

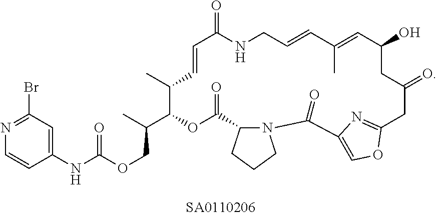 [SMILES: CC1=C\[C@@H](O)CC(=O)Cc2nc(co2)C(=O)N2CCC[C@@H]2C(=O)O[C@H]([C@H](C)COC(=O)Nc2ccnc(Br)c2)[C@H](C)/C=C/C(=O)NC\C=C\1]

In embodiments, the compound is

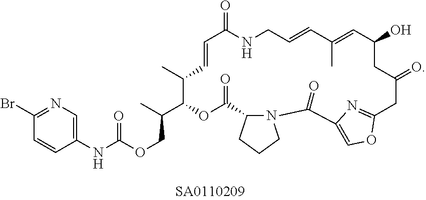 [SMILES: CC1=C\[C@@H](O)CC(=O)Cc2nc(co2)C(=O)N2CCC[C@@H]2C(=O)O[C@H]([C@H](C)COC(=O)Nc2ccc(Br)nc2)[C@H](C)/C=C/C(=O)NC\C=C\1]

In embodiments, the compound is

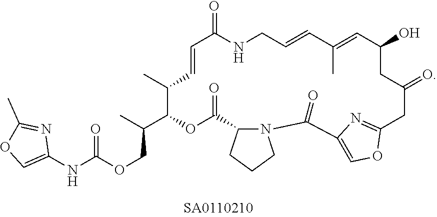 [SMILES: CC1=C\[C@@H](O)CC(=O)Cc2nc(co2)C(=O)N2CCC[C@@H]2C(=O)O[C@H]([C@H](C)COC(=O)Nc2coc(C)n2)[C@H](C)/C=C/C(=O)NC\C=C\1]

In embodiments, the compound is

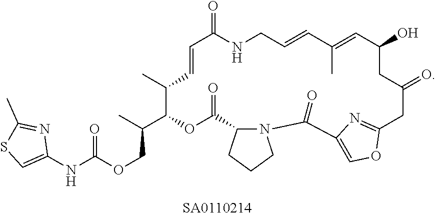 [SMILES: CC1=C\[C@@H](O)CC(=O)Cc2nc(co2)C(=O)N2CCC[C@@H]2C(=O)O[C@H]([C@H](C)COC(=O)Nc2csc(C)n2)[C@H](C)/C=C/C(=O)NC\C=C\1]

In embodiments, the compound is

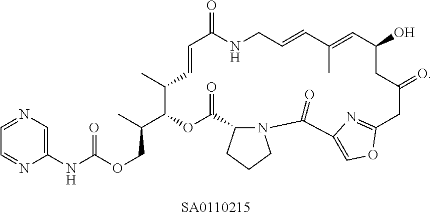 [SMILES: CC1=C\[C@@H](O)CC(=O)Cc2nc(co2)C(=O)N2CCC[C@@H]2C(=O)O[C@H]([C@H](C)COC(=O)Nc2cnccn2)[C@H](C)/C=C/C(=O)NC\C=C\1]

In embodiments, the compound is

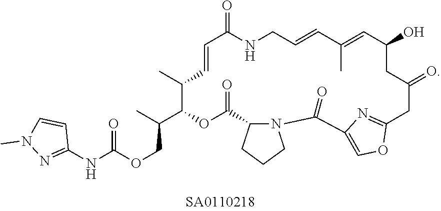 [SMILES: CC1=C\[C@@H](O)CC(=O)Cc2nc(co2)C(=O)N2CCC[C@@H]2C(=O)O[C@H]([C@H](C)COC(=O)Nc2ccn(C)n2)[C@H](C)/C=C/C(=O)NC\C=C\1]

In embodiments, the compound is

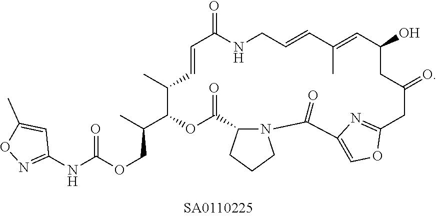 [SMILES: CC1=C\[C@@H](O)CC(=O)Cc2nc(co2)C(=O)N2CCC[C@@H]2C(=O)O[C@H]([C@H](C)COC(=O)Nc2cc(C)on2)[C@H](C)/C=C/C(=O)NC\C=C\1]

In embodiments, the compound is

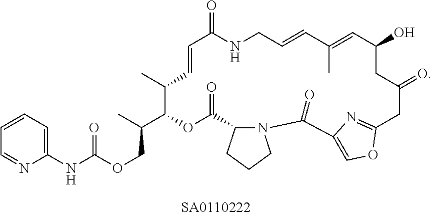 [SMILES: CC1=C\[C@@H](O)CC(=O)Cc2nc(co2)C(=O)N2CCC[C@@H]2C(=O)O[C@H]([C@H](C)COC(=O)Nc2ccccn2)[C@H](C)/C=C/C(=O)NC\C=C\1]

In embodiments, the compound is

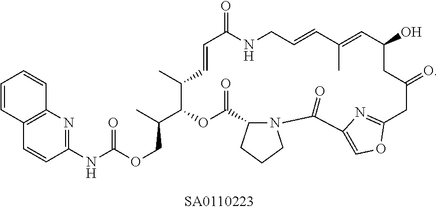 [SMILES: CC1=C\[C@@H](O)CC(=O)Cc2nc(co2)C(=O)N2CCC[C@@H]2C(=O)O[C@H]([C@H](C)COC(=O)Nc2ccc3ccccc3n2)[C@H](C)/C=C/C(=O)NC\C=C\1]

In embodiments, the compound is

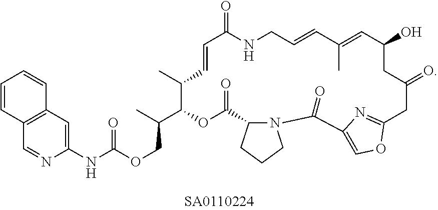 [SMILES: CC1=C\[C@@H](O)CC(=O)Cc2nc(co2)C(=O)N2CCC[C@@H]2C(=O)O[C@H]([C@H](C)COC(=O)Nc2cc3ccccc3cn2)[C@H](C)/C=C/C(=O)NC\C=C\1]

In embodiments, the compound is

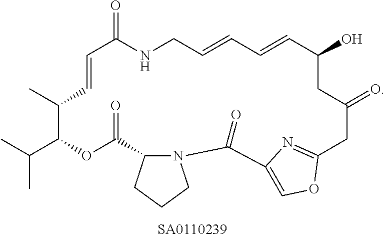 [SMILES: CC(C)[C@H]1OC(=O)[C@H]2CCCN2C(=O)c2coc(n2)CC(=O)C[C@H](O)/C=C/C=C/CNC(=O)/C=C/[C@H]1C]

In embodiments, the compound is

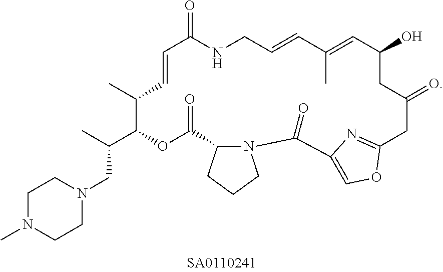 [SMILES: CC1=C\[C@@H](O)CC(=O)Cc2nc(co2)C(=O)N2CCC[C@@H]2C(=O)O[C@H]([C@@H](C)CN2CCN(C)CC2)[C@H](C)/C=C/C(=O)NC\C=C\1]

In embodiments, the compound is

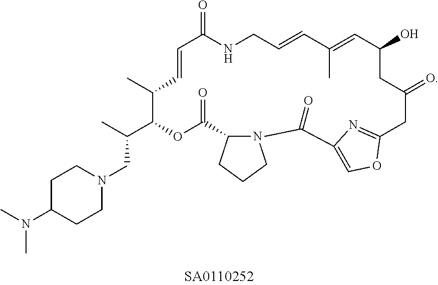 [SMILES: CC1=C\[C@@H](O)CC(=O)Cc2nc(co2)C(=O)N2CCC[C@@H]2C(=O)O[C@H]([C@@H](C)CN2CCC(N(C)C)CC2)[C@H](C)/C=C/C(=O)NC\C=C\1]

In embodiments, the compound is

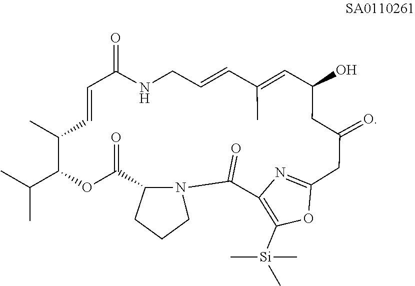 [SMILES: CC1=C\[C@@H](O)CC(=O)Cc2nc(c([Si](C)(C)C)o2)C(=O)N2CCC[C@@H]2C(=O)O[C@H](C(C)C)[C@H](C)/C=C/C(=O)NC\C=C\1]

In embodiments, the compound is

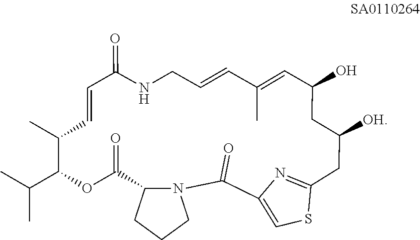 [SMILES: CC1=C\[C@@H](O)C[C@@H](O)Cc2nc(cs2)C(=O)N2CCC[C@@H]2C(=O)O[C@H](C(C)C)[C@H](C)/C=C/C(=O)NC\C=C\1]

In embodiments, the compound is

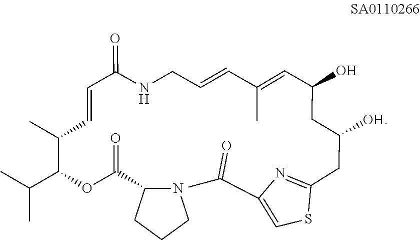 [SMILES: CC(=O)c1csc(C[C@@H](O)C[C@H](O)/C=C(C)/C=C/CNC(=O)/C=C/[C@@H](C)[C@H](OC(=O)[C@H]2CCCN2)C(C)C)n1]

In embodiments, the compound is

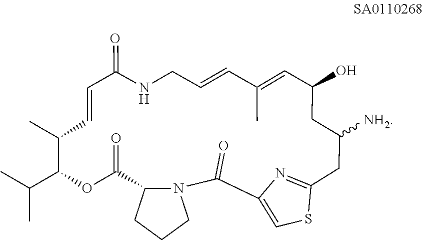 [SMILES: CC1=C\[C@@H](O)CC(N)Cc2nc(cs2)C(=O)N2CCC[C@@H]2C(=O)O[C@H](C(C)C)[C@H](C)/C=C/C(=O)NC\C=C\1]

In embodiments, the compound is

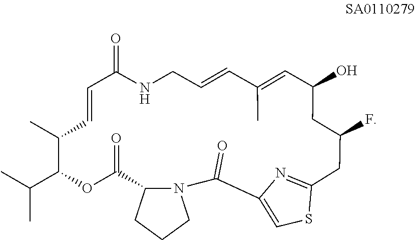 [SMILES: CC1=C\[C@@H](O)C[C@@H](F)Cc2nc(cs2)C(=O)N2CCC[C@@H]2C(=O)O[C@H](C(C)C)[C@H](C)/C=C/C(=O)NC\C=C\1]

In embodiments, the compound is

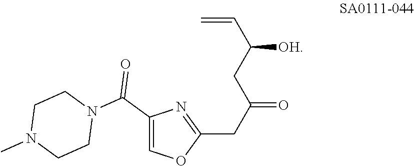 [SMILES: C=C[C@@H](O)CC(=O)Cc1nc(C(=O)N2CCN(C)CC2)co1]

In embodiments, the compound is

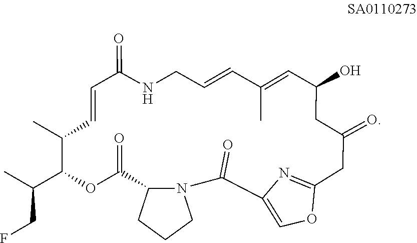 [SMILES: CC1=C\[C@@H](O)CC(=O)Cc2nc(co2)C(=O)N2CCC[C@@H]2C(=O)O[C@H]([C@H](C)CF)[C@H](C)/C=C/C(=O)NC\C=C\1]

In embodiments, the compound is

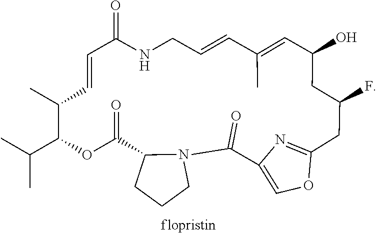 [SMILES: CC1=C\[C@@H](O)C[C@@H](F)Cc2nc(co2)C(=O)N2CCC[C@@H]2C(=O)O[C@H](C(C)C)[C@H](C)/C=C/C(=O)NC\C=C\1]

In embodiments, the compound is

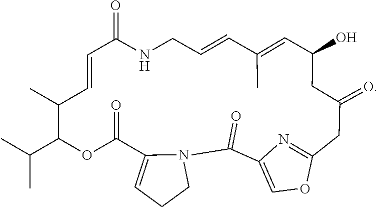 [SMILES: CC1=C\[C@@H](O)CC(=O)Cc2nc(co2)C(=O)N2CCC=C2C(=O)OC(C(C)C)C(C)/C=C/C(=O)NC\C=C\1]

In embodiments, the compound is

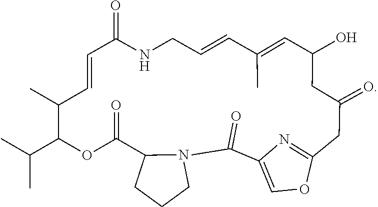 [SMILES: CC1=C\C(O)CC(=O)Cc2nc(co2)C(=O)N2CCCC2C(=O)OC(C(C)C)C(C)/C=C/C(=O)NC\C=C\1]

In embodiments, the compound is

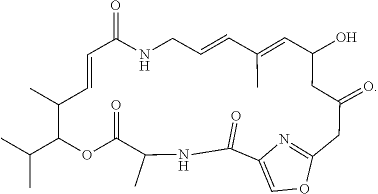 [SMILES: CC1=C\C(O)CC(=O)Cc2nc(co2)C(=O)NC(C)C(=O)OC(C(C)C)C(C)/C=C/C(=O)NC\C=C\1]

In embodiments, the compound is

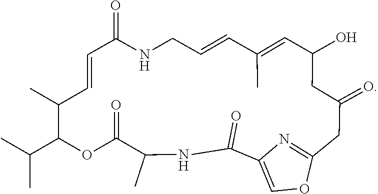 [SMILES: CC1=C\C(O)CC(=O)Cc2nc(co2)C(=O)NC(C)C(=O)OC(C(C)C)C(C)/C=C/C(=O)NC\C=C\1]

In embodiments, the compound is

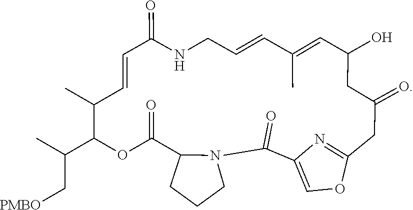

In embodiments, the compound is

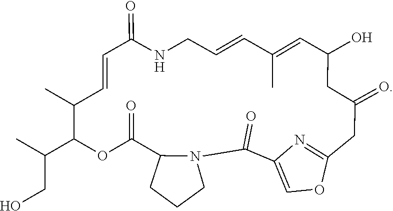 [SMILES: CC1=C\C(O)CC(=O)Cc2nc(co2)C(=O)N2CCCC2C(=O)OC(C(C)CO)C(C)/C=C/C(=O)NC\C=C\1]

In embodiments, the compound is

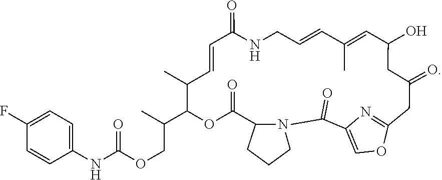 [SMILES: CC1=C\C(O)CC(=O)Cc2nc(co2)C(=O)N2CCCC2C(=O)OC(C(C)COC(=O)Nc2ccc(F)cc2)C(C)/C=C/C(=O)NC\C=C\1]

In embodiments, the compound is

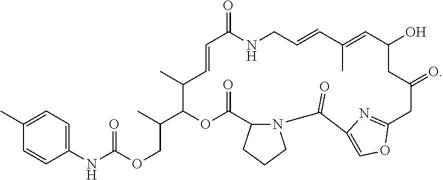 [SMILES: CC1=C\C(O)CC(=O)Cc2nc(co2)C(=O)N2CCCC2C(=O)OC(C(C)COC(=O)Nc2ccc(C)cc2)C(C)/C=C/C(=O)NC\C=C\1]

In embodiments, the compound is

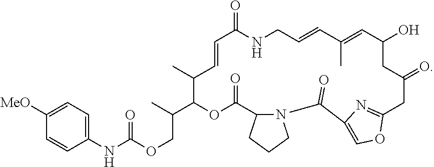 [SMILES: COc1ccc(NC(=O)OCC(C)C2OC(=O)C3CCCN3C(=O)c3coc(n3)CC(=O)CC(O)/C=C(C)/C=C/CNC(=O)/C=C/C2C)cc1]

In embodiments, the compound is

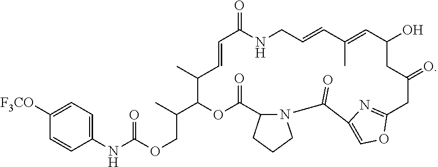 [SMILES: CC1=C\C(O)CC(=O)Cc2nc(co2)C(=O)N2CCCC2C(=O)OC(C(C)COC(=O)Nc2ccc(OC(F)(F)F)cc2)C(C)/C=C/C(=O)NC\C=C\1]

In embodiments, the compound is

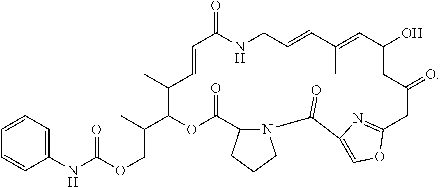 [SMILES: CC1=C\C(O)CC(=O)Cc2nc(co2)C(=O)N2CCCC2C(=O)OC(C(C)COC(=O)Nc2ccccc2)C(C)/C=C/C(=O)NC\C=C\1]

In embodiments, the compound is

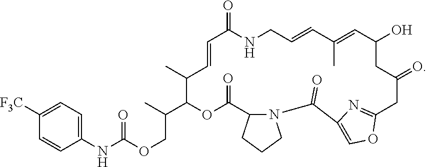 [SMILES: CC1=C\C(O)CC(=O)Cc2nc(co2)C(=O)N2CCCC2C(=O)OC(C(C)COC(=O)Nc2ccc(C(F)(F)F)cc2)C(C)/C=C/C(=O)NC\C=C\1]

In embodiments, the compound is

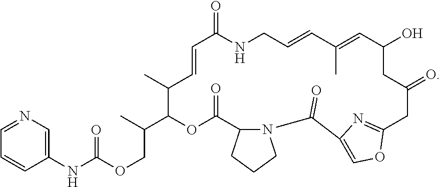 [SMILES: CC1=C\C(O)CC(=O)Cc2nc(co2)C(=O)N2CCCC2C(=O)OC(C(C)COC(=O)Nc2cccnc2)C(C)/C=C/C(=O)NC\C=C\1]

In embodiments, the compound is

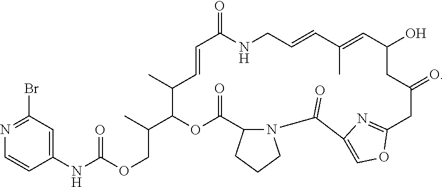 [SMILES: CC1=C\C(O)CC(=O)Cc2nc(co2)C(=O)N2CCCC2C(=O)OC(C(C)COC(=O)Nc2ccnc(Br)c2)C(C)/C=C/C(=O)NC\C=C\1]

In embodiments, the compound is

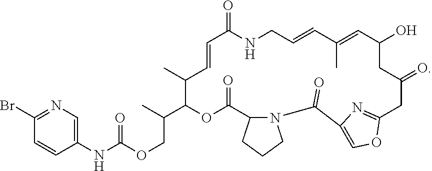 [SMILES: CC1=C\C(O)CC(=O)Cc2nc(co2)C(=O)N2CCCC2C(=O)OC(C(C)COC(=O)Nc2ccc(Br)nc2)C(C)/C=C/C(=O)NC\C=C\1]

In embodiments, the compound is

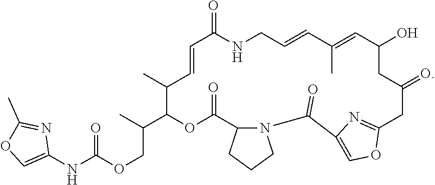 [SMILES: CC1=C\C(O)CC(=O)Cc2nc(co2)C(=O)N2CCCC2C(=O)OC(C(C)COC(=O)Nc2coc(C)n2)C(C)/C=C/C(=O)NC\C=C\1]

In embodiments, the compound is

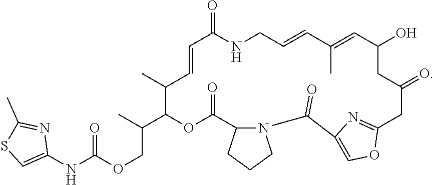 [SMILES: CC1=C\C(O)CC(=O)Cc2nc(co2)C(=O)N2CCCC2C(=O)OC(C(C)COC(=O)Nc2csc(C)n2)C(C)/C=C/C(=O)NC\C=C\1]

In embodiments, the compound is

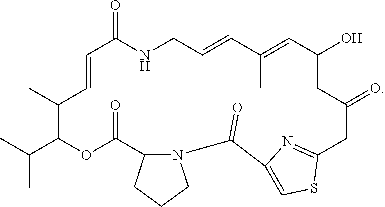 [SMILES: CC1=C\C(O)CC(=O)Cc2nc(cs2)C(=O)N2CCCC2C(=O)OC(C(C)C)C(C)/C=C/C(=O)NC\C=C\1]

In embodiments, the compound is

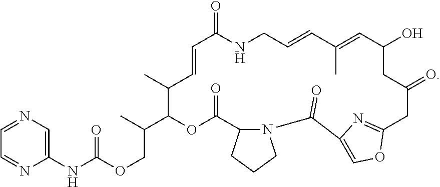 [SMILES: CC1=C\C(O)CC(=O)Cc2nc(co2)C(=O)N2CCCC2C(=O)OC(C(C)COC(=O)Nc2cnccn2)C(C)/C=C/C(=O)NC\C=C\1]

In embodiments, the compound is

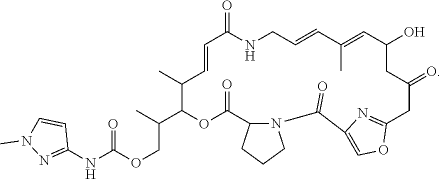 [SMILES: CC1=C\C(O)CC(=O)Cc2nc(co2)C(=O)N2CCCC2C(=O)OC(C(C)COC(=O)Nc2ccn(C)n2)C(C)/C=C/C(=O)NC\C=C\1]

In embodiments, the compound is

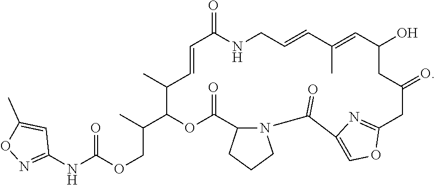 [SMILES: CC1=C\C(O)CC(=O)Cc2nc(co2)C(=O)N2CCCC2C(=O)OC(C(C)COC(=O)Nc2cc(C)on2)C(C)/C=C/C(=O)NC\C=C\1]

In embodiments, the compound is

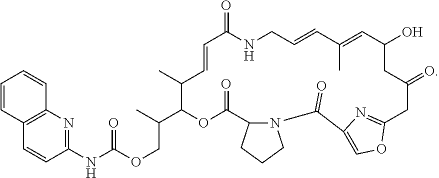 [SMILES: CC1=C\C(O)CC(=O)Cc2nc(co2)C(=O)N2CCCC2C(=O)OC(C(C)COC(=O)Nc2ccc3ccccc3n2)C(C)/C=C/C(=O)NC\C=C\1]

In embodiments, the compound is

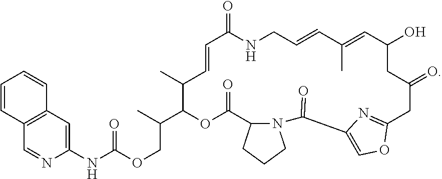 [SMILES: CC1=C\C(O)CC(=O)Cc2nc(co2)C(=O)N2CCCC2C(=O)OC(C(C)COC(=O)Nc2cc3ccccc3cn2)C(C)/C=C/C(=O)NC\C=C\1]

In embodiments, the compound is

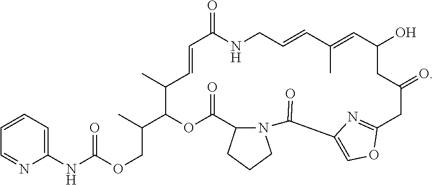 [SMILES: CC1=C\C(O)CC(=O)Cc2nc(co2)C(=O)N2CCCC2C(=O)OC(C(C)COC(=O)Nc2ccccn2)C(C)/C=C/C(=O)NC\C=C\1]

In embodiments, the compound is

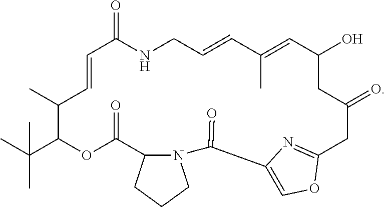 [SMILES: CC1=C\C(O)CC(=O)Cc2nc(co2)C(=O)N2CCCC2C(=O)OC(C(C)(C)C)C(C)/C=C/C(=O)NC\C=C\1]

In embodiments, the compound is

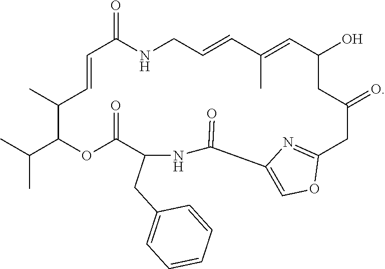 [SMILES: CC1=C\C(O)CC(=O)Cc2nc(co2)C(=O)NC(Cc2ccccc2)C(=O)OC(C(C)C)C(C)/C=C/C(=O)NC\C=C\1]

In embodiments, the compound is

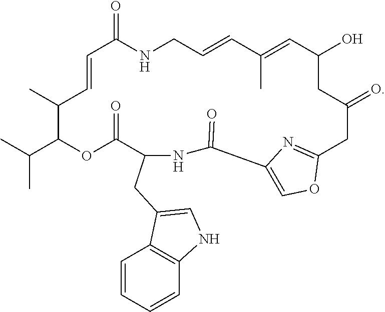 [SMILES: CC1=C\C(O)CC(=O)Cc2nc(co2)C(=O)NC(Cc2c[nH]c3ccccc23)C(=O)OC(C(C)C)C(C)/C=C/C(=O)NC\C=C\1]

In embodiments, the compound is

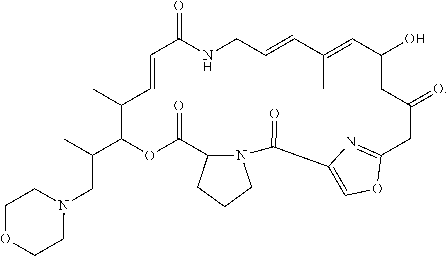 [SMILES: CC1=C\C(O)CC(=O)Cc2nc(co2)C(=O)N2CCCC2C(=O)OC(C(C)CN2CCOCC2)C(C)/C=C/C(=O)NC\C=C\1]

In embodiments, the compound is

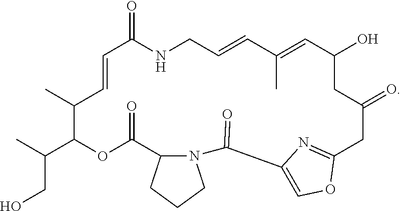 [SMILES: CC1=C\C(O)CC(=O)Cc2nc(co2)C(=O)N2CCCC2C(=O)OC(C(C)CO)C(C)/C=C/C(=O)NC\C=C\1]

In embodiments, the compound is

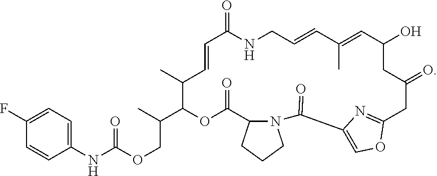 [SMILES: CC1=C\C(O)CC(=O)Cc2nc(co2)C(=O)N2CCCC2C(=O)OC(C(C)COC(=O)Nc2ccc(F)cc2)C(C)/C=C/C(=O)NC\C=C\1]

In embodiments, the compound is

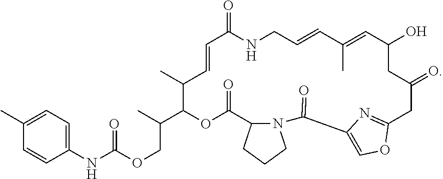 [SMILES: CC1=C\C(O)CC(=O)Cc2nc(co2)C(=O)N2CCCC2C(=O)OC(C(C)COC(=O)Nc2ccc(C)cc2)C(C)/C=C/C(=O)NC\C=C\1]

In embodiments, the compound is

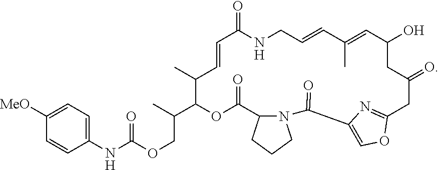 [SMILES: COc1ccc(NC(=O)OCC(C)C2OC(=O)C3CCCN3C(=O)c3coc(n3)CC(=O)CC(O)/C=C(C)/C=C/CNC(=O)/C=C/C2C)cc1]

In embodiments, the compound is

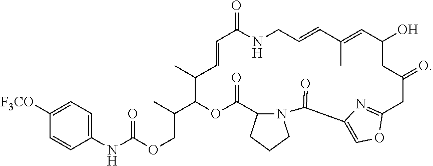 [SMILES: CC1=C\C(O)CC(=O)Cc2nc(co2)C(=O)N2CCCC2C(=O)OC(C(C)COC(=O)Nc2ccc(OC(F)(F)F)cc2)C(C)/C=C/C(=O)NC\C=C\1]

In embodiments, the compound is

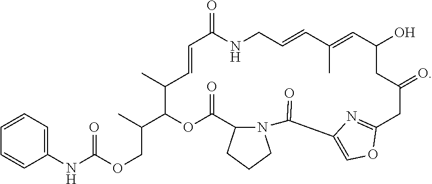 [SMILES: CC1=C\C(O)CC(=O)Cc2nc(co2)C(=O)N2CCCC2C(=O)OC(C(C)COC(=O)Nc2ccccc2)C(C)/C=C/C(=O)NC\C=C\1]

In embodiments, the compound is

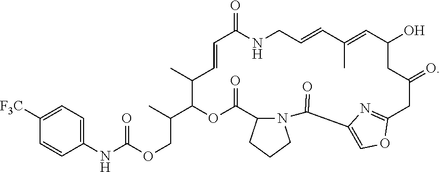 [SMILES: CC1=C\C(O)CC(=O)Cc2nc(co2)C(=O)N2CCCC2C(=O)OC(C(C)COC(=O)Nc2ccc(C(F)(F)F)cc2)C(C)/C=C/C(=O)NC\C=C\1]

In embodiments, the compound is

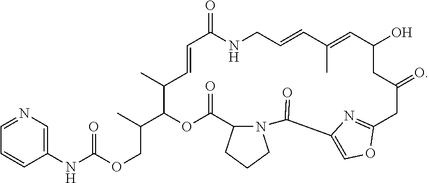 [SMILES: CC1=C\C(O)CC(=O)Cc2nc(co2)C(=O)N2CCCC2C(=O)OC(C(C)COC(=O)Nc2cccnc2)C(C)/C=C/C(=O)NC\C=C\1]

In embodiments, the compound is

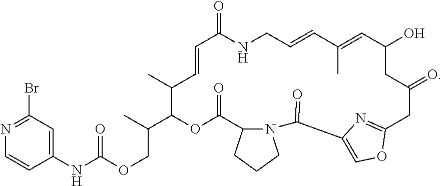 [SMILES: CC1=C\C(O)CC(=O)Cc2nc(co2)C(=O)N2CCCC2C(=O)OC(C(C)COC(=O)Nc2ccnc(Br)c2)C(C)/C=C/C(=O)NC\C=C\1]

In embodiments, the compound is

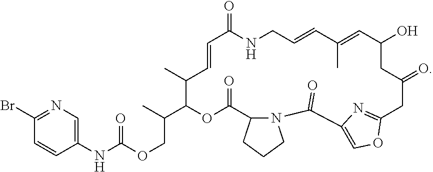 [SMILES: CC1=C\C(O)CC(=O)Cc2nc(co2)C(=O)N2CCCC2C(=O)OC(C(C)COC(=O)Nc2ccc(Br)nc2)C(C)/C=C/C(=O)NC\C=C\1]

In embodiments, the compound is

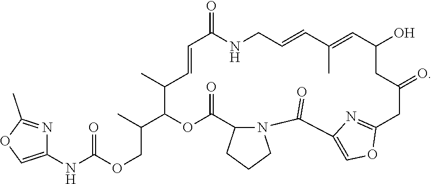 [SMILES: CC1=C\C(O)CC(=O)Cc2nc(co2)C(=O)N2CCCC2C(=O)OC(C(C)COC(=O)Nc2coc(C)n2)C(C)/C=C/C(=O)NC\C=C\1]

In embodiments, the compound is

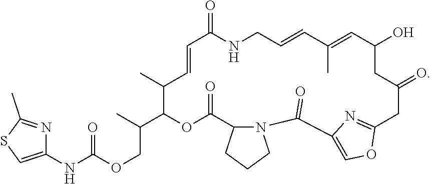 [SMILES: CC1=C\C(O)CC(=O)Cc2nc(co2)C(=O)N2CCCC2C(=O)OC(C(C)COC(=O)Nc2csc(C)n2)C(C)/C=C/C(=O)NC\C=C\1]

In embodiments, the compound is

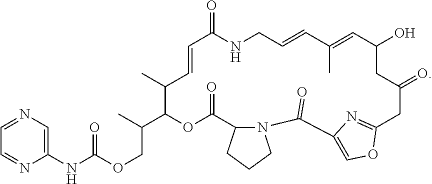 [SMILES: CC1=C\C(O)CC(=O)Cc2nc(co2)C(=O)N2CCCC2C(=O)OC(C(C)COC(=O)Nc2cnccn2)C(C)/C=C/C(=O)NC\C=C\1]

In embodiments, the compound is

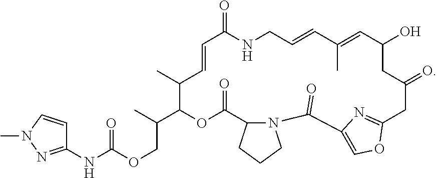 [SMILES: CC1=C\C(O)CC(=O)Cc2nc(co2)C(=O)N2CCCC2C(=O)OC(C(C)COC(=O)Nc2ccn(C)n2)C(C)/C=C/C(=O)NC\C=C\1]

In embodiments, the compound is

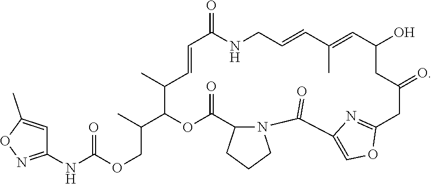 [SMILES: CC1=C\C(O)CC(=O)Cc2nc(co2)C(=O)N2CCCC2C(=O)OC(C(C)COC(=O)Nc2cc(C)on2)C(C)/C=C/C(=O)NC\C=C\1]

In embodiments, the compound is

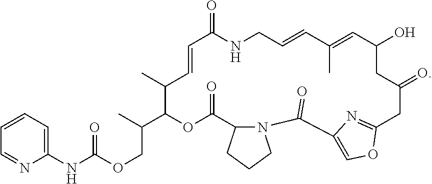 [SMILES: CC1=C\C(O)CC(=O)Cc2nc(co2)C(=O)N2CCCC2C(=O)OC(C(C)COC(=O)Nc2ccccn2)C(C)/C=C/C(=O)NC\C=C\1]

In embodiments, the compound is

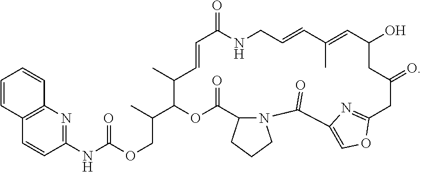 [SMILES: CC1=C\C(O)CC(=O)Cc2nc(co2)C(=O)N2CCCC2C(=O)OC(C(C)COC(=O)Nc2ccc3ccccc3n2)C(C)/C=C/C(=O)NC\C=C\1]

In embodiments, the compound is

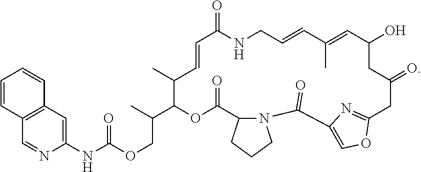 [SMILES: CC1=C\C(O)CC(=O)Cc2nc(co2)C(=O)N2CCCC2C(=O)OC(C(C)COC(=O)Nc2cc3ccccc3cn2)C(C)/C=C/C(=O)NC\C=C\1]

In embodiments, the compound is

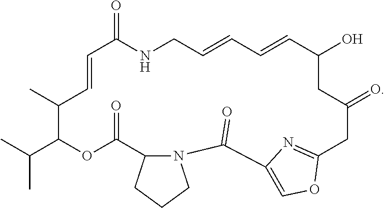 [SMILES: CC(C)C1OC(=O)C2CCCN2C(=O)c2coc(n2)CC(=O)CC(O)/C=C/C=C/CNC(=O)/C=C/C1C]

In embodiments, the compound is

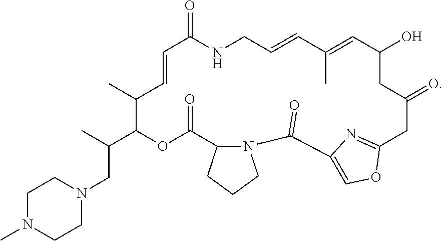 [SMILES: CC1=C\C(O)CC(=O)Cc2nc(co2)C(=O)N2CCCC2C(=O)OC(C(C)CN2CCN(C)CC2)C(C)/C=C/C(=O)NC\C=C\1]

In embodiments, the compound is

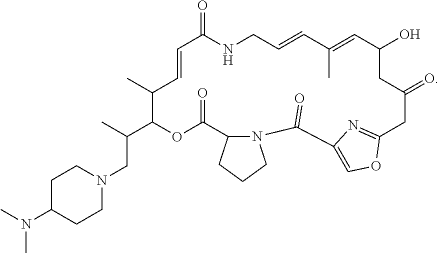 [SMILES: CC1=C\C(O)CC(=O)Cc2nc(co2)C(=O)N2CCCC2C(=O)OC(C(C)CN2CCC(N(C)C)CC2)C(C)/C=C/C(=O)NC\C=C\1]

In embodiments, the compound is

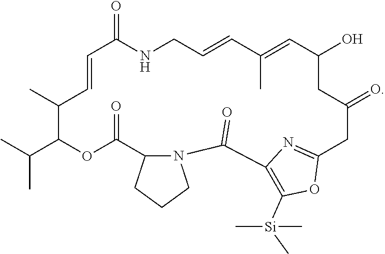 [SMILES: CC1=C\C(O)CC(=O)Cc2nc(c([Si](C)(C)C)o2)C(=O)N2CCCC2C(=O)OC(C(C)C)C(C)/C=C/C(=O)NC\C=C\1]

In embodiments, the compound is

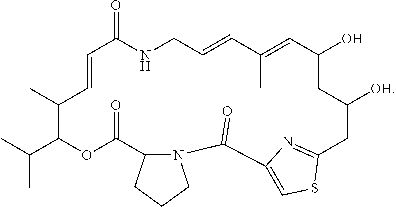 [SMILES: CC1=C\C(O)CC(O)Cc2nc(cs2)C(=O)N2CCCC2C(=O)OC(C(C)C)C(C)/C=C/C(=O)NC\C=C\1]

In embodiments, the compound is

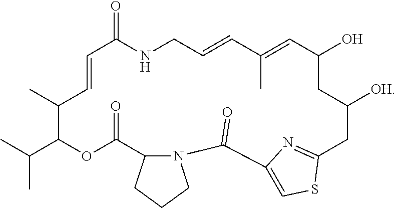 [SMILES: CC1=C\C(O)CC(O)Cc2nc(cs2)C(=O)N2CCCC2C(=O)OC(C(C)C)C(C)/C=C/C(=O)NC\C=C\1]

In embodiments, the compound is

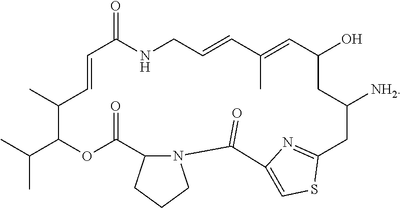 [SMILES: CC1=C\C(O)CC(N)Cc2nc(cs2)C(=O)N2CCCC2C(=O)OC(C(C)C)C(C)/C=C/C(=O)NC\C=C\1]

In embodiments, the compound is

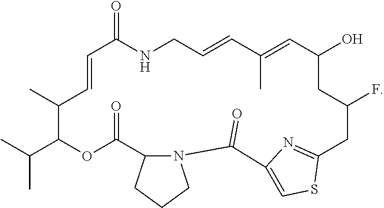 [SMILES: CC1=C\C(O)CC(F)Cc2nc(cs2)C(=O)N2CCCC2C(=O)OC(C(C)C)C(C)/C=C/C(=O)NC\C=C\1]

In embodiments, the compound is

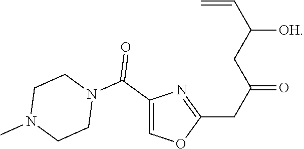 [SMILES: C=CC(O)CC(=O)Cc1nc(C(=O)N2CCN(C)CC2)co1]

In embodiments, the compound is

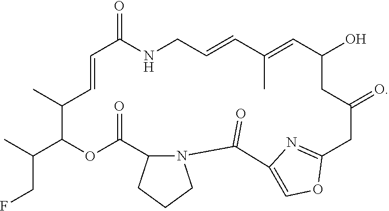 [SMILES: CC1=C\C(O)CC(=O)Cc2nc(co2)C(=O)N2CCCC2C(=O)OC(C(C)CF)C(C)/C=C/C(=O)NC\C=C\1]

In embodiments, the compound is

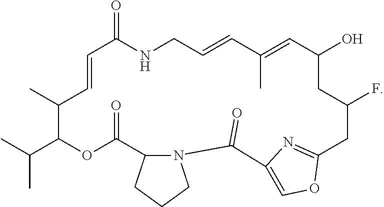 [SMILES: CC1=C\C(O)CC(F)Cc2nc(co2)C(=O)N2CCCC2C(=O)OC(C(C)C)C(C)/C=C/C(=O)NC\C=C\1]

In embodiments, the compound is

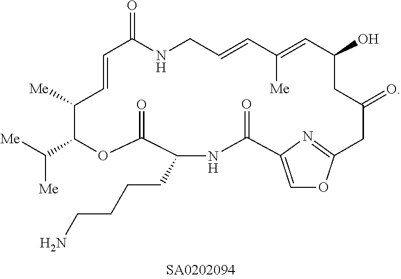 [SMILES: CC1=C\[C@@H](O)CC(=O)Cc2nc(co2)C(=O)N[C@H](CCCCN)C(=O)O[C@H](C(C)C)[C@H](C)/C=C/C(=O)NC\C=C\1]

In embodiments, the compound is

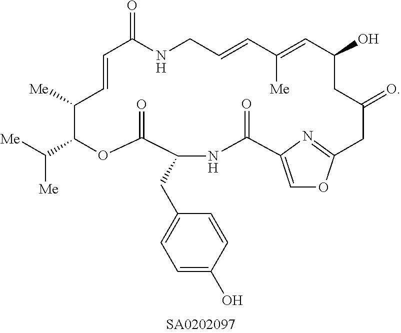 [SMILES: CC1=C\[C@@H](O)CC(=O)Cc2nc(co2)C(=O)N[C@H](Cc2ccc(O)cc2)C(=O)O[C@H](C(C)C)[C@H](C)/C=C/C(=O)NC\C=C\1]

In embodiments, the compound is

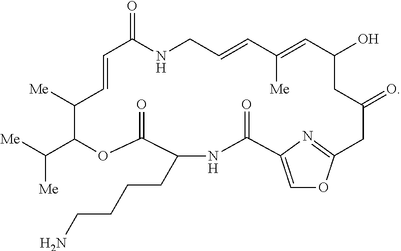 [SMILES: CC1=C\C(O)CC(=O)Cc2nc(co2)C(=O)NC(CCCCN)C(=O)OC(C(C)C)C(C)/C=C/C(=O)NC\C=C\1]

In embodiments, the compound is

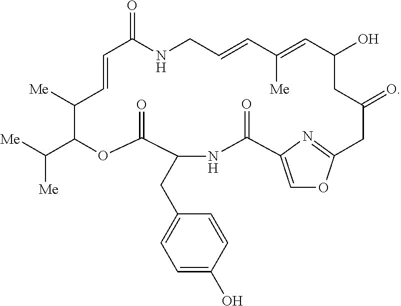 [SMILES: CC1=C\C(O)CC(=O)Cc2nc(co2)C(=O)NC(Cc2ccc(O)cc2)C(=O)OC(C(C)C)C(C)/C=C/C(=O)NC\C=C\1]

In embodiments, the compound is not a compound described in U.S. Pat. No. 5,587,475. In embodiments, the compound is not

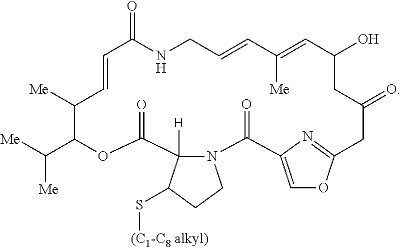

In embodiments, the compound does not have the formula:

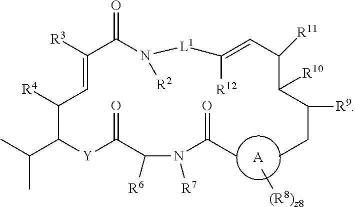

In embodiments, the compound does not have the formula:

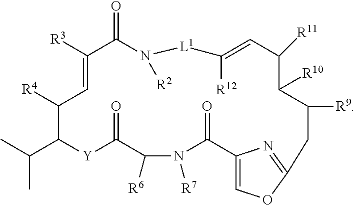

In embodiments, the compound is a compound described herein (e.g., in the Compound section, Example Section, Method Section, or in a claim, table or figure).

### III. Methods

In an aspect is a method of making a compound, or salt thereof, having the formula:

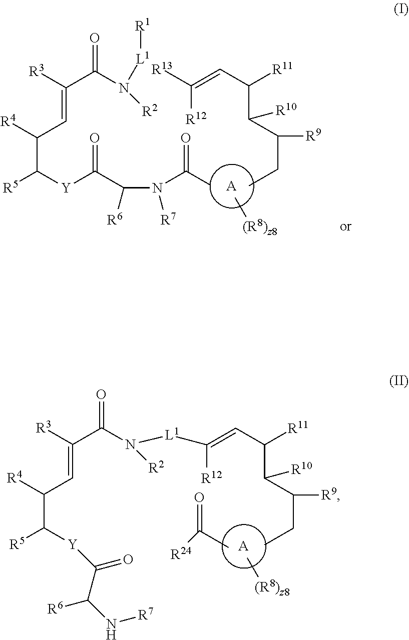

the method including mixing compound A and compound B together in a reaction vessel; wherein compound A has the formula:

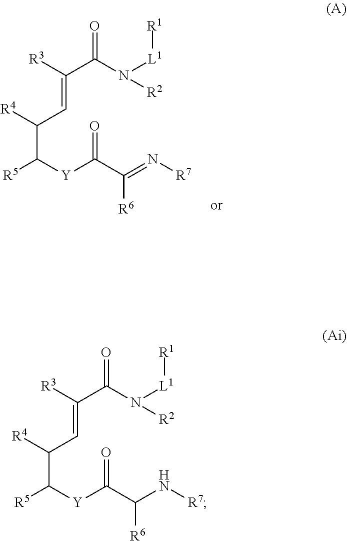

and compound B has the formula:

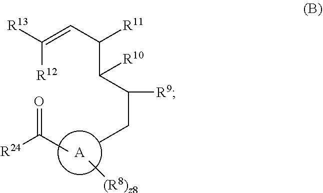

wherein R¹, R¹³, and R²⁴ are each independently a bioconjugate reactive moiety or leaving group; Y is —O— or —NH—; L¹ is a bond, substituted or unsubstituted alkylene, or substituted or unsubstituted heteroalkylene; R² is hydrogen or unsubstituted C₁-C₃ alkyl; R³, R⁴, and R⁵ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —OPO₃H, —OSO₃H, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or an amino acid side chain; R⁷ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —CCl₃, —COOH, —CH₂COOH, —CONH₂, —OH, —SH, —NO₂, —NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶ and R⁷ may optionally be joined to form a substituted or unsubstituted heterocycloalkyl or substituted or unsubstituted heteroaryl; R⁸ is oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; Ring A is cycloalkylene, heterocycloalkylene, arylene, or heteroarylene; z8 is an integer from 0 to 10; R⁹, R¹⁰, R¹¹, and R¹² are each independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, the method further includes exposing the compound of Formula (I) or Formula (II) to reaction conditions conducive to form compound D (e.g., reacting R¹ and R¹³ to form compound D), wherein compound D has the formula:

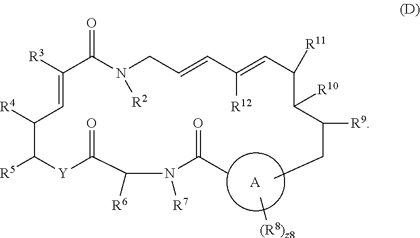

In embodiments, the method further includes exposing the compound of Formula (I) or Formula (II) to conditions conducive to form compound D (e.g., allowing R¹ and R¹³ react), wherein compound D has the formula:

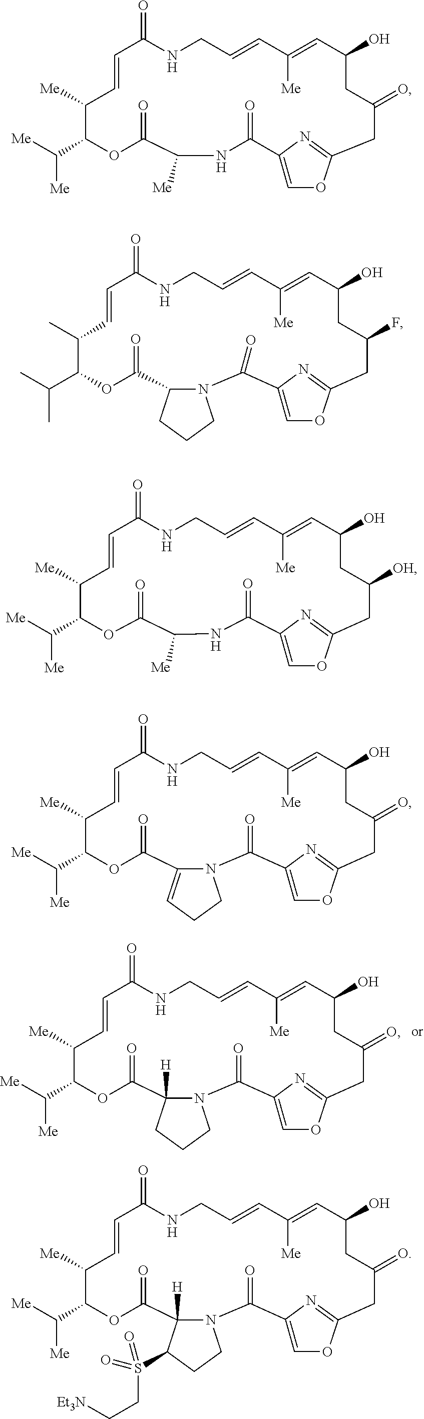

In embodiments, compound A is made by mixing (e.g., allowing the compounds to contact) compound A1 and compound A2 or compound A2i in a reaction vessel; wherein compound A1 has the formula:

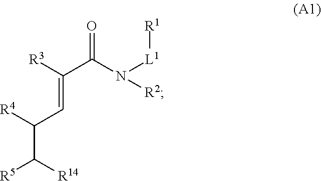

compound A2 has the formula:

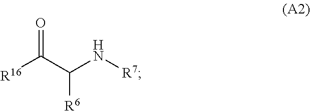

and compound A2 has the formula:

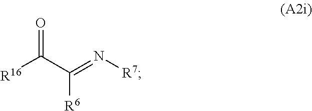

wherein R¹⁴ and R¹⁶ are independently a bioconjugate reactive moiety or leaving group.

In embodiments, compound A1 is made by mixing compound A3, compound A4, and compound A5 in a reaction vessel; wherein compound A3 has the formula:

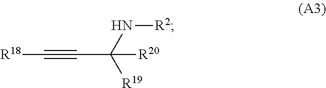

compound A4 has the formula:

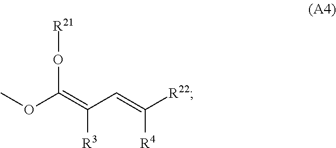

compound A5 has the formula:

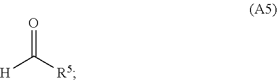

wherein R¹⁸, R¹⁹, R²⁰, and R²² are independently hydrogen, substituted or unsubstituted alkyl, substituted or unsubstituted heteroalkyl; and R²¹ is hydrogen or a protecting group. R³, R⁴, and R⁵ are as described herein.

In embodiments, compound Ali is made by mixing compound A3, compound A4i, and compound A5 in a reaction vessel; wherein compound A1i has the formula:

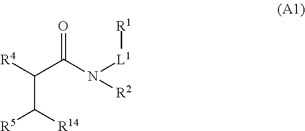

compound A3 has the formula:

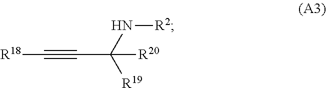

compound A4i has the formula:

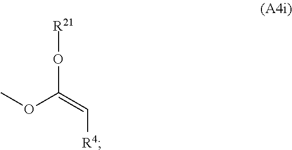

compound A5 has the formula:

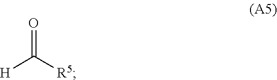

wherein R¹⁸, R¹⁹, R²⁰, and R²² are independently hydrogen, substituted or unsubstituted alkyl, substituted or unsubstituted heteroalkyl; and R²¹ is hydrogen or a protecting group. R³, R⁴, and R⁵ are as described herein.

In embodiments, compound B is made by mixing compound B1 and compound B2; wherein compound B1 has the formula:

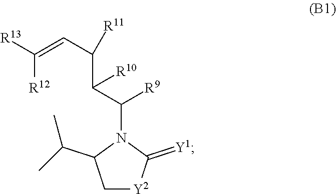

and compound B2 has the formula:

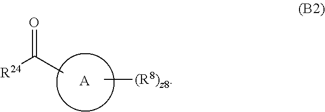

The symbol Y¹ is ═O or S. The symbol Y² is —O— or —S—; and R²⁴ is a bioconjugate reactive moiety or leaving group.

In embodiments compound B1 is made by mixing compound B3 and compound B4, wherein compound B3 has the formula:

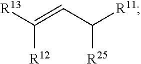

compound B4 has the formula:

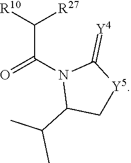

The symbol Y⁴ is ═O or S. The symbol Y⁵ is —O— or —S—; and R²⁵ and R²⁷ are independently a bioconjugate reactive moiety or leaving group. In embodiments, Y⁴ is ═O. In embodiments, Y⁴ is ═S. In embodiments, Y⁵ is —O—. In embodiments, Y⁵ is —S—.

In an aspect is provided a method of treating an infectious disease, the method including administering to a subject in need thereof an effective amount of a compound as described herein, including embodiments.

In embodiments, the method includes a metal catalyst.

In embodiments, the method includes a Ru-based olefin metathesis catalyst, as depicted in the scheme below, for example:

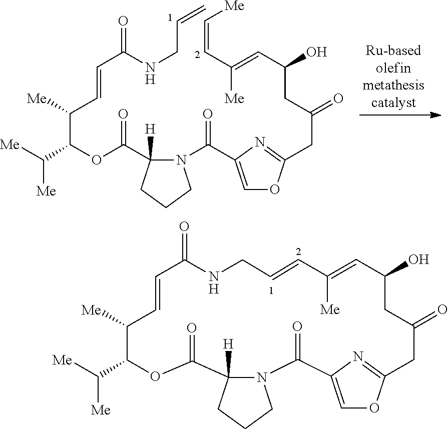

One of ordinary skill in the art would understand the use of certain solvents and/or catalysts may be included in methods described herein. In embodiments, the reaction vessel includes TfOH, dichloromethane (DCM), isopropanol, propargylamine, AlMe₃, CuCN, n-BuLi, Bu₃SnH, N,N′-Dicyclohexylcarbodiimide (DCC), 4-Dimethylaminopyridine (DMAP), Et₂NH, TiCl₄, Pr₂EtN 2,6-lutidine, tert-butyldimethylsilyl ether triflate (TBSOTf), THF, 1-[Bis(dimethylamino) methylene]-1H-1,2,3-triazolo[4,5-b]pyridinium 3-oxid hexafluorophosphate (HATU), JackiePhos, Tris(dibenzylideneacetone)dipalladium (Pd₂dba₃), toluene, Tetra-n-butylammonium fluoride (BiuNF), imidazole (Im.HCl), Et₂BOMe, NaBH₄, MeOH, water, or trifluoromethanesulfonate (TfO).

In embodiments, the reaction occurs at different temperatures for different periods of time. In embodiments, the reaction occurs at a temperature less than about −50° C. In embodiments, the reaction occurs at a temperature less than about −60° C. In embodiments, the reaction occurs at a temperature less than about −70° C. In embodiments, the reaction occurs at a temperature about −78° C. In embodiments, the reaction occurs at a temperature about 0° C. to 23° C. In embodiments, the reaction occurs at a temperature about 40° C.

In embodiments, the infectious disease is a bacterial infection. In embodiments, the infectious disease is a group A streptococcus infection (e.g., Streptococcus pyogenes infection). In embodiments, the infectious disease is a gram-positive bacterial infection. In embodiments, the infectious disease is a Staphylococcus aureus infection. In embodiments, the infectious disease is Gram-positive or Gram-negative bacterial infection. In embodiments, the infectious disease is an infection associated with S. aureus, E. facium, E. faecalis, K. pneumonoiaea, H. influenzaea, or P. aeruginosa. In embodiments, the infectious disease is a S. aureus, E. facium, E. faecalis, K. pneumonoiaea, H. influenzaea, or P. aeruginosa infection. In embodiments, the treatment includes inhibiting bacterial growth. In embodiments, the treatment reduces bacterial reproduction, relative to a control. In embodiments, the treatment does not kill the bacterial cell. In embodiments, the infectious disease is a bacteria associated disease.

In embodiments, the infectious disease is a bacteria associated disease (e.g., actinomycosis, anthrax, abscesses in tissues (e.g., mouth in gastrointestinal tract, pelvic cavity, or lungs), whooping cough, lyme disease, brucellosis, enteritis, Guillain-Barre syndrome, pneumonia, conjunctivitis, trachoma, botulism, pseudomembranous colitis, food poisoning, tetanus, diphtheria, ehrlichiosis, bacterial endocarditis, urinary tract infection, diarrhea, meningitis (e.g., bacterial meningitis), sepsis, fever, tularemia, bronchitis, peptic ulcer, gastritis, Legionnaire's disease, Pontiac fever, leptospirosis, listeriosis, leprosy, gonorrhea, opthalmia, nocardiosis, typhoid fever, salmonellosis, shigellosis, impetigo, cystitis, Scarlet fever, syphilis, cholera, or plague.

In embodiments, the bacteria associated disease is actinomycosis, anthrax, abscesses in tissues (e.g., mouth in gastrointestinal tract, pelvic cavity, or lungs), whooping cough, lyme disease, brucellosis, enteritis, Guillain-Barre syndrome, pneumonia, conjunctivitis, trachoma, botulism, pseudomembranous colitis, food poisoning, tetanus, diphtheria, ehrlichiosis, bacterial endocarditis, urinary tract infection, diarrhea, meningitis (e.g., bacterial meningitis), sepsis, fever, tularemia, bronchitis, peptic ulcer, gastritis, Legionnaire's disease, Pontiac fever, leptospirosis, listeriosis, leprosy, gonorrhea, opthalmia, nocardiosis, typhoid fever, salmonellosis, shigellosis, impetigo, cystitis, Scarlet fever, syphilis, cholera, or plague.

In embodiments, the bacteria associated disease is actinomycosis. In embodiments, the bacteria associated disease is anthrax. In embodiments, the bacteria associated disease is abscesses in tissues (e.g., mouth in gastrointestinal tract, pelvic cavity, or lungs). In embodiments, the bacteria associated disease is whooping cough. In embodiments, the bacteria associated disease is lyme disease. In embodiments, the bacteria associated disease is brucellosis. In embodiments, the bacteria associated disease is enteritis. In embodiments, the bacteria associated disease is Guillain-Barre syndrome. In embodiments, the bacteria associated disease is pneumonia. In embodiments, the bacteria associated disease is conjunctivitis. In embodiments, the bacteria associated disease is trachoma. In embodiments, the bacteria associated disease is botulism. In embodiments, the bacteria associated disease is pseudomembranous colitis. In embodiments, the bacteria associated disease is food poisoning. In embodiments, the bacteria associated disease is tetanus. In embodiments, the bacteria associated disease is diphtheria. In embodiments, the bacteria associated disease is ehrlichiosis. In embodiments, the bacteria associated disease is bacterial endocarditis. In embodiments, the bacteria associated disease is urinary tract infection. In embodiments, the bacteria associated disease is diarrhea. In embodiments, the bacteria associated disease is meningitis (e.g., bacterial meningitis). In embodiments, the bacteria associated disease is sepsis. In embodiments, the bacteria associated disease is fever. In embodiments, the bacteria associated disease is tularemia. In embodiments, the bacteria associated disease is bronchitis. In embodiments, the bacteria associated disease is peptic ulcer. In embodiments, the bacteria associated disease is gastritis. In embodiments, the bacteria associated disease is Legionnaire's disease. In embodiments, the bacteria associated disease is Pontiac fever. In embodiments, the bacteria associated disease is leptospirosis. In embodiments, the bacteria associated disease is listeriosis. In embodiments, the bacteria associated disease is leprosy. In embodiments, the bacteria associated disease is gonorrhea. In embodiments, the bacteria associated disease is opthalmia. In embodiments, the bacteria associated disease is nocardiosis. In embodiments, the bacteria associated disease is typhoid fever. In embodiments, the bacteria associated disease is salmonellosis. In embodiments, the bacteria associated disease is shigellosis. In embodiments, the bacteria associated disease is impetigo. In embodiments, the bacteria associated disease is cystitis. In embodiments, the bacteria associated disease is Scarlet fever. In embodiments, the bacteria associated disease is syphilis. In embodiments, the bacteria associated disease is cholera. In embodiments, the bacteria associated disease is plague.

In embodiments, the the bacterial infection is a Staphylococcus infection, an Enterococcus infection, an Acinetobacter infection, a Bacillus infection, a Streptococcus infection, an Escherichia infection, a Pseudomonas infection, a Klebsiella infection, or a Haemophilus infection.

In embodiments, the method includes the compound (e.g., a compound described herein) binding to the peptidyl transferase domain of the 50s ribosomal subunit.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### IV. Embodiments

### Embodiment P1

A method of making a compound, or salt thereof, having the formula:

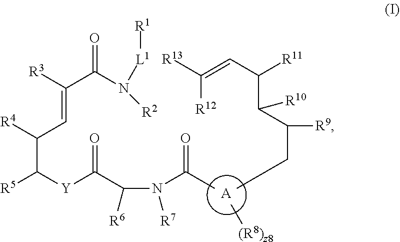

said method comprising mixing compound A and compound B together in a reaction vessel; wherein compound A has the formula:

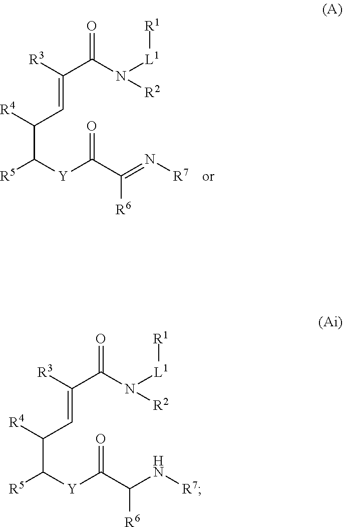

and compound B has the formula:

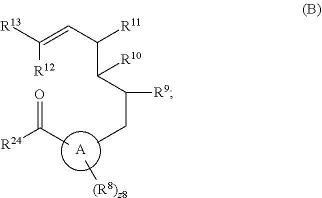

wherein R¹, R¹³, and R²⁴ are leaving groups; Y is —O— or —NH—; L¹ is a bond, substituted or unsubstituted alkylene, or substituted or unsubstituted heteroalkylene; R² is hydrogen or unsubstituted C₁-C₃ alkyl; R³, R⁴, and R⁵ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl; R⁷ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —CCl₃, —COOH, —CH₂COOH, —CONH₂, —OH, —SH, —NO₂, —NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶ and R⁷ may optionally be joined to form a substituted or unsubstituted heterocycloalkyl or heteroaryl; R⁸ is oxo, halogen, —CCl₂, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; Ring A is cycloalkylene, heterocycloalkylene, arylene, or heteroarylene; z8 is an integer from 0 to 10; R⁹ is hydrogen, oxo, halogen, —CN, —OH, —NH₂, —COOH, —CONH₂, —NO₂, —SH, —SO₃H, —N₃, unsubstituted alkyl, or unsubstituted heteroalkyl; R¹⁰ and R¹² are independently hydrogen, substituted or unsubstituted C₁-C₃ alkyl, or substituted or unsubstituted 2 to 3 membered heteroalkyl; R¹¹ is hydrogen, oxo, halogen, —CCl₂, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₂, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment P2

A method of making a compound, or salt thereof, having the formula:

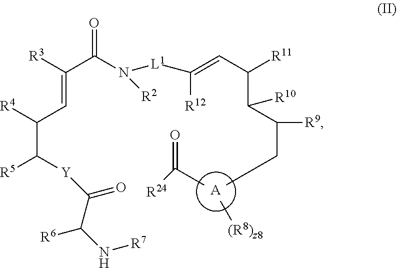

said method comprising mixing compound A and compound B together in a reaction vessel; wherein compound A has the formula:

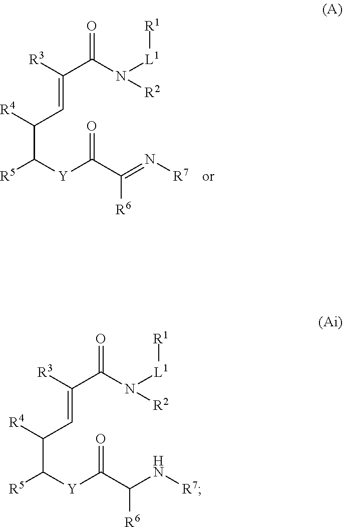

and compound B has the formula:

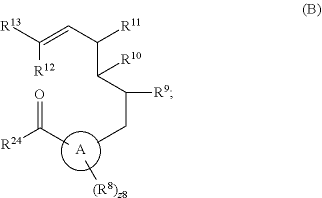

wherein R¹, R¹³, and R²⁴ are leaving groups; Y is —O— or —NH—; L¹ is a bond, substituted or unsubstituted alkylene, or substituted or unsubstituted heteroalkylene; R² is hydrogen or unsubstituted C₁-C₃ alkyl; R³, R⁴, and R⁵ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl; R⁷ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —CCl₃, —COOH, —CH₂COOH, —CONH₂, —OH, —SH, —NO₂, —NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶ and R⁷ may optionally be joined to form a substituted or unsubstituted heterocycloalkyl or heteroaryl; R⁸ is oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; Ring A is cycloalkylene, heterocycloalkylene, arylene, or heteroarylene; z8 is an integer from 0 to 10; R⁹ is hydrogen, oxo, halogen, —CN, —OH, —NH₂, —COOH, —CONH₂, —NO₂, —SH, —SO₃H, —N₃, unsubstituted alkyl, or unsubstituted heteroalkyl; R¹⁰ and R¹² are independently hydrogen, substituted or unsubstituted C₁-C₃ alkyl, or substituted or unsubstituted 2 to 3 membered heteroalkyl; R¹¹ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₂, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment P3

The method of embodiments P1 or P2, further comprising exposing the compound of Formula (I) or Formula (II) to conditions conducive to reacting R¹ and R¹³ to form compound D, wherein compound D has the formula:

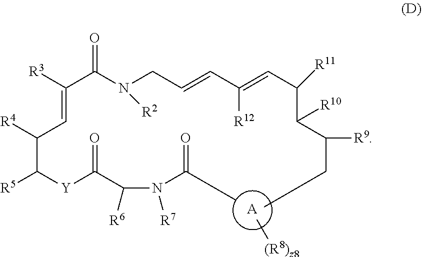

### Embodiment P5

The method of embodiments P1 or P2, wherein L¹ is a substituted or unsubstituted alkenylene.

### Embodiment P6

The method of embodiments P1 or P2, wherein L¹ is a substituted or unsubstituted C₁-C₃ alkenylene.

### Embodiment P7

The method of any one of embodiments P1, P2, or P4 to P6, wherein R¹ is an organotin heteroalkyl.

### Embodiment P8

The method of any one of embodiments P1, P2, or P4 to P6, wherein R¹ is

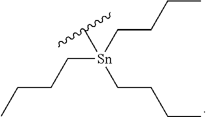 [SMILES: [*][Sn]([CH2]CCC)([CH2]CCC)[CH2]CCC]

### Embodiment P9

The method of any one of embodiments P1 to P8, wherein R² is hydrogen or unsubstituted methyl.

### Embodiment P10

The method of any one of embodiments P1 to P8, wherein R² is hydrogen.

### Embodiment P11

The method of any one of embodiments P1 to P12, wherein R³ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment P12

The method of any one of embodiments P1 to P12, wherein R³ is hydrogen, substituted or unsubstituted cycloalkyl, substituted or unsubstituted heterocycloalkyl, substituted or unsubstituted aryl, or substituted or unsubstituted heteroaryl.

### Embodiment P13

The method of any one of embodiments P1 to P12, wherein R⁴ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment P14

The method of any one of embodiments P1 to P12, wherein R⁴ is a substituted or unsubstituted C₁-C₆ alkyl.

Embodiment P15. The method of any one of embodiments P1 to P12, wherein R⁴ is an unsubstituted C₁-C₃ alkyl.

### Embodiment P16

The method of any one of embodiments P1 to P12, wherein R⁴ is an unsubstituted methyl.

### Embodiment P17

The method of any one of embodiments P1 to P16, wherein R⁵ is a substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment P18

The method of any one of embodiments P1 to P16, wherein R⁵ is an unsubstituted C₁-C₆ alkyl.

### Embodiment P19

The method of any one of embodiments P1 to P16, wherein R⁵ is an unsubstituted C₃ alkyl.

### Embodiment P20

The method of any one of embodiments P1 to P16, wherein R⁵ is an unsubstituted isopropyl.

### Embodiment 21

The method of any one of embodiments 1 to 20, wherein R⁶ is an unsubstituted C₁-C₃ alkyl.

### Embodiment P22

The method of any one of embodiments P1 to P20, wherein R⁶ is an unsubstituted methyl.

### Embodiment P23

The method of any one of embodiments P1 to P20, wherein R⁶ and R⁷ are joined to form a substituted or unsubstituted heterocycloalkyl or heteroaryl.

### Embodiment P24

The method of any one of embodiments P1 to P20, wherein R⁶ and R⁷ are joined to form a substituted or unsubstituted 3 to 6 membered heterocycloalkyl, or substituted or unsubstituted 5 to 6 membered heteroaryl.

### Embodiment P25

The method of any one of embodiments P1 to P20, wherein R⁶ and R⁷ are joined to form a substituted or unsubstituted 3 to 6 membered heterocycloalkyl.

### Embodiment P26

The method of any one of embodiments IP to P20, wherein R⁶ and R⁷ are joined to form a substituted or unsubstituted pyrrolidinyl or 2,3-dihydropyrrolyl.

### Embodiment P27

The method of any one of embodiments P1 to P22, wherein R⁷ is hydrogen or substituted or unsubstituted C₁-C₆ alkyl.

### Embodiment P28

The method of any one of embodiments P1 to P22, wherein R⁷ is hydrogen.

### Embodiment P29

The method of any one of embodiments P1 to P28, wherein Ring A is a substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene.

### Embodiment P30

The method of any one of embodiments P1 to P28, wherein Ring A is a substituted or unsubstituted 5 to 6 membered heteroarylene.

### Embodiment P31

The method of any one of embodiments P1 to P28, wherein Ring A is a substituted or unsubstituted oxazolylene.

### Embodiment P32

The method of any one of embodiments P1 to P31, wherein z8 is 0.

### Embodiment P33

The method of any one of embodiments P1 to P32, wherein R⁹ is oxo or halogen.

### Embodiment P34

The method of any one of embodiments P1 to P33, wherein R⁹ is oxo or —F.

### Embodiment P35

The method of any one of embodiments P1 to P34, wherein R¹⁰ is hydrogen or unsubstituted methyl.

### Embodiment P36

The method of any one of embodiments P1 to P35, wherein R¹¹ is halogen, —OH, or —NH₂.

### Embodiment P37

The method of any one of embodiments P1 to P36, wherein R¹² is hydrogen or an unsubstituted C₁-C₃ alkyl.

### Embodiment P38

The method of any one of embodiments P1 to P36, wherein R¹² is hydrogen.

### Embodiment P39

The method of any one of embodiments P1 to P36, wherein R¹³ is —Br.

### Embodiment P40

The method of embodiments P1 or P2, wherein compound A has the formula:

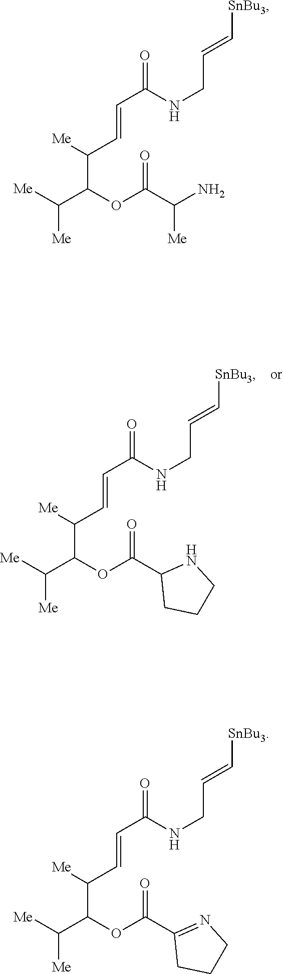

### Embodiment P41

The method of embodiments P1 or P2, wherein compound B has the formula:

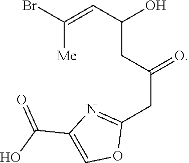 [SMILES: C/C(Br)=C\C(O)CC(=O)Cc1nc(C(=O)O)co1]

### Embodiment P42

The method of embodiments P1 or P2, further comprising exposing the compound of Formula (I) or Formula (II) to conditions conducive to reacting R¹ and R¹³ to form compound D, wherein compound D has the formula:

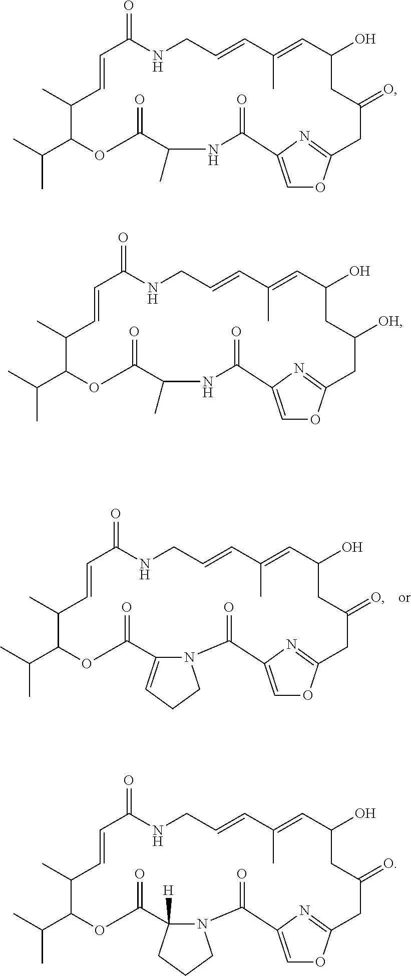 [SMILES: CC1=C\C(O)CC(=O)Cc2nc(co2)C(=O)NC(C)C(=O)OC(C(C)C)C(C)/C=C/C(=O)NC\C=C\1 ; CC1=C\C(O)CC(O)Cc2nc(co2)C(=O)NC(C)C(=O)OC(C(C)C)C(C)/C=C/C(=O)NC\C=C\1 ; CC1=C\C(O)CC(=O)Cc2nc(co2)C(=O)N2CCC=C2C(=O)OC(C(C)C)C(C)/C=C/C(=O)NC\C=C\1 ; CC1=C\C(O)CC(=O)Cc2nc(co2)C(=O)N2CCC[C@@H]2C(=O)OC(C(C)C)C(C)/C=C/C(=O)NC\C=C\1]

### Embodiment P43

The method of embodiments P1 or P2, wherein compound A is made by mixing compound A1 and compound A2 in a reaction vessel; wherein compound A1 has the formula:

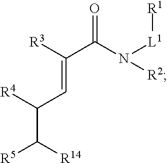

and compound A2 has the formula:

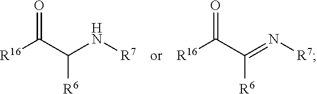

wherein R¹⁴ and R¹⁶ are leaving groups.

### Embodiment P44

The method of embodiment P43, wherein compound A1 is made by mixing compound A3, compound A4, and compound A5 in a reaction vessel; wherein compound A3 has the formula:

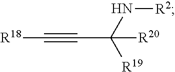

compound A4 has the formula:

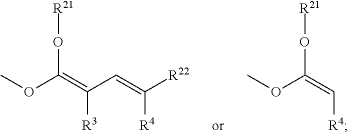

compound A5 has the formula:

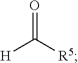

wherein R¹⁸, R¹⁹, R²⁰, and R²² are independently hydrogen, substituted or unsubstituted alkyl, substituted or unsubstituted heteroalkyl; R³, R⁴, and R⁵ are independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R²¹ is hydrogen or a protecting group.

### Embodiment P45

The method of embodiments P1 or P2, wherein compound B is made by mixing compound B1 and compound B2; wherein compound B1 has the formula:

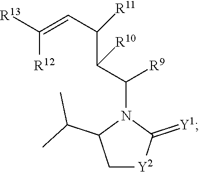

and compound B2 has the formula:

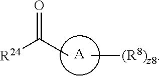

The symbol Y¹ is ═O or S. The symbol Y² is —O— or —S—; and R²⁴ is a leaving group.

### Embodiment P46

The method of embodiment P45, wherein compound B2 has the formula:

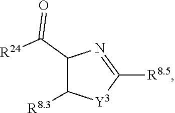

wherein Y³ is —S—, —O—, or —NR^{8.4}-; R^{8.4} is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —CCl₃, —COOH, —CH₂COOH, —CONH₂, —OH, —SH, substituted or unsubstituted alkyl, or substituted or unsubstituted heteroalkyl; and R^{8.3} and R^{8.5} are independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, or substituted or unsubstituted heteroalkyl.

### Embodiment P47

The method of embodiment P45, wherein compound B1 is made by mixing compound B3 and compound B4, wherein compound B3 has the formula:

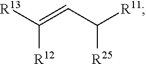

compound B4 has the formula:

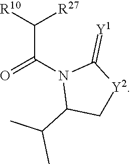

The symbol Y¹ is ═O or S. The symbol Y² is —O— or —S—; and R²⁵ and R²⁷ are leaving groups.

### Embodiment P48

A compound, or salt thereof, having the formula:

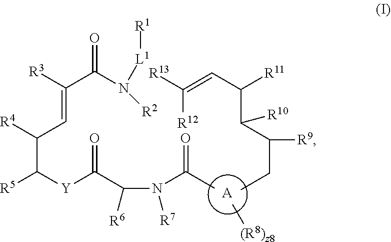

wherein R¹ and R¹³ are leaving groups; Y is —O— or —NH—; L¹ is a bond, substituted or unsubstituted alkylene, or substituted or unsubstituted heteroalkylene; R² is hydrogen or unsubstituted C₁-C₃ alkyl; R³, R⁴, and R⁵ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl; R⁷ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —CCl₃, —COOH, —CH₂COOH, —CONH₂, —OH, —SH, —NO₂, —NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶ and R⁷ may optionally be joined to form a substituted or unsubstituted heterocycloalkyl or heteroaryl; R⁸ is oxo, halogen, —CCl₂, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; Ring A is cycloalkylene, heterocycloalkylene, arylene, or heteroarylene; z8 is an integer from 0 to 10; R⁹ is hydrogen, oxo, halogen, —CN, —OH, —NH₂, —COOH, —CONH₂, —NO₂, —SH, —SO₃H, —N₃, unsubstituted alkyl, or unsubstituted heteroalkyl; R¹⁰ and R¹² are independently hydrogen, substituted or unsubstituted C₁-C₃ alkyl, or substituted or unsubstituted 2 to 3 membered heteroalkyl; and R¹¹ is hydrogen, oxo, halogen, —CCl₂, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₂, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment P49

A compound, or salt thereof, having the formula:

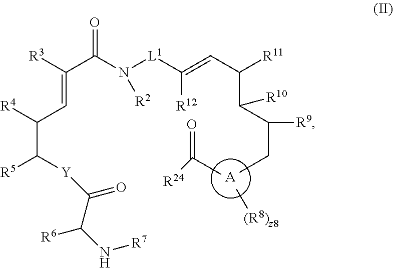

wherein R¹ and R¹³ are leaving groups; Y is —O— or —NH—; L¹ is a bond, substituted or unsubstituted alkylene, or substituted or unsubstituted heteroalkylene; R² is hydrogen or unsubstituted C₁-C₃ alkyl; R³, R⁴, and R⁵ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl; R⁷ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —CCl₃, —COOH, —CH₂COOH, —CONH₂, —OH, —SH, —NO₂, —NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶ and R⁷ may optionally be joined to form a substituted or unsubstituted heterocycloalkyl or heteroaryl; R⁸ is oxo, halogen, —CCl₂, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; Ring A is cycloalkylene, heterocycloalkylene, arylene, or heteroarylene; z8 is an integer from 0 to 10; R⁹ is hydrogen, oxo, halogen, —CN, —OH, —NH₂, —COOH, —CONH₂, —NO₂, —SH, —SO₃H, —N₃, unsubstituted alkyl, or unsubstituted heteroalkyl; R¹⁰ and R¹² are independently hydrogen, substituted or unsubstituted C₁-C₃ alkyl, or substituted or unsubstituted 2 to 3 membered heteroalkyl; and R¹¹ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment P50

A compound, or salt thereof, having the formula:

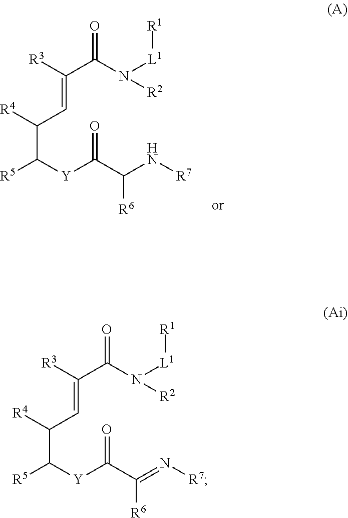

wherein R¹ is a leaving group; Y is —O— or —NH—; L¹ is a bond, substituted or unsubstituted alkylene, or substituted or unsubstituted heteroalkylene; R² is hydrogen or unsubstituted C₁-C₃ alkyl; R³, R⁴, and R⁵ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl; R⁷ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —CCl₃, —COOH, —CH₂COOH, —CONH₂, —OH, —SH, —NO₂, —NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R⁶ and R⁷ may optionally be joined to form a substituted or unsubstituted heterocycloalkyl or heteroaryl.

### Embodiment P51

A compound, or salt thereof, having the formula:

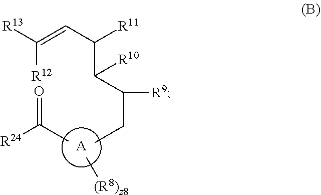

wherein R¹³ and R²⁴ are leaving groups; R⁸ is oxo, halogen, —CCl₂, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; Ring A is cycloalkylene, heterocycloalkylene, arylene, or heteroarylene; z8 is an integer from 0 to 10; R⁹ is hydrogen, oxo, halogen, —CN, —OH, —NH₂, —COOH, —CONH₂, —NO₂, —SH, —SO₃H, —N₃, unsubstituted alkyl, or unsubstituted heteroalkyl; R¹⁰ and R¹² are independently hydrogen, substituted or unsubstituted C₁-C₃ alkyl, or substituted or unsubstituted 2 to 3 membered heteroalkyl; and R¹¹ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment P52

A compound, or salt thereof, having the formula:

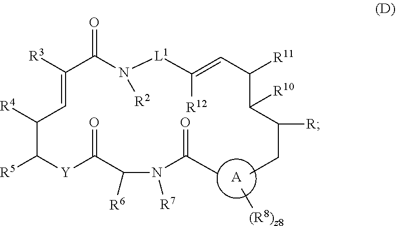

wherein Y is —O— or —NH—; L¹ is a bond, substituted or unsubstituted alkylene, or substituted or unsubstituted heteroalkylene; R² is hydrogen or unsubstituted C₁-C₃ alkyl; R³, R⁴, and R⁵ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl; R⁷ is hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —CCl₃, —COOH, —CH₂COOH, —CONH₂, —OH, —SH, —NO₂, —NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶ and R⁷ may optionally be joined to form a substituted or unsubstituted heterocycloalkyl or heteroaryl; R⁸ is oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; Ring A is cycloalkylene, heterocycloalkylene, arylene, or heteroarylene; z8 is an integer from 0 to 10; R⁹ is hydrogen, oxo, halogen, —CN, —OH, —NH₂, —COOH, —CONH₂, —NO₂, —SH, —SO₃H, —N₃, unsubstituted alkyl, or unsubstituted heteroalkyl; R¹⁰ and R¹² are independently hydrogen, substituted or unsubstituted C₁-C₃ alkyl, or substituted or unsubstituted 2 to 3 membered heteroalkyl; and R¹¹ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₂, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; wherein the compound does not have the formula:

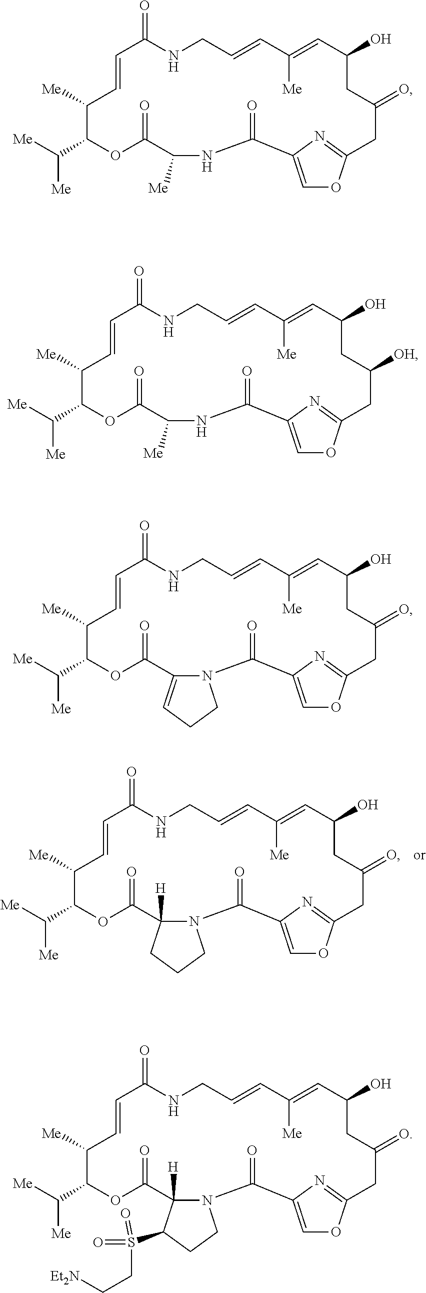

### Embodiment P53

The compound of embodiment P52, wherein the compound has the formula:

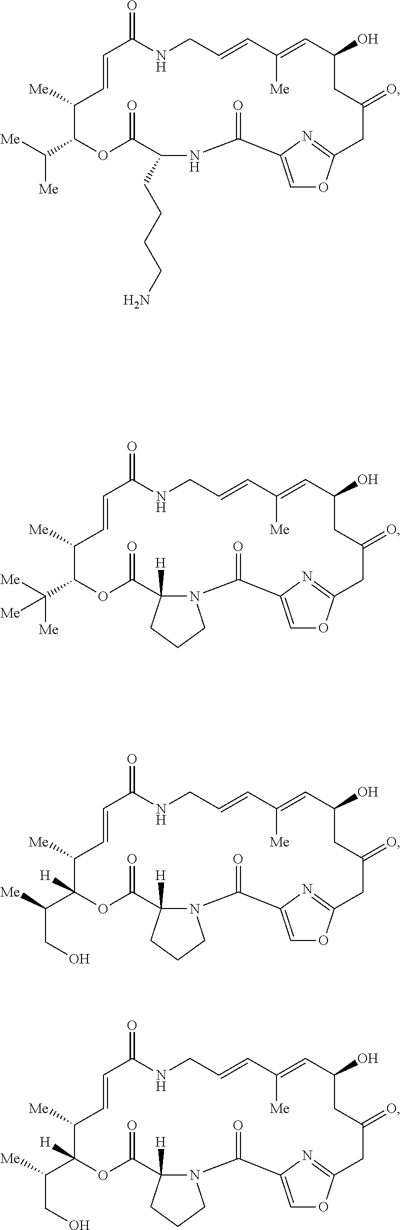 [SMILES: CC1=C\[C@@H](O)CC(=O)Cc2nc(co2)C(=O)N[C@H](CCCCN)C(=O)O[C@H](C(C)C)[C@H](C)/C=C/C(=O)NC\C=C\1 ; CC1=C\[C@@H](O)CC(=O)Cc2nc(co2)C(=O)N2CCC[C@@H]2C(=O)O[C@H](C(C)(C)C)[C@H](C)/C=C/C(=O)NC\C=C\1 ; CC1=C\[C@@H](O)CC(=O)Cc2nc(co2)C(=O)N2CCC[C@@H]2C(=O)O[C@H]([C@H](C)CO)[C@H](C)/C=C/C(=O)NC\C=C\1 ; CC1=C\[C@@H](O)CC(=O)Cc2nc(co2)C(=O)N2CCC[C@@H]2C(=O)O[C@H]([C@@H](C)CO)[C@H](C)/C=C/C(=O)NC\C=C\1]

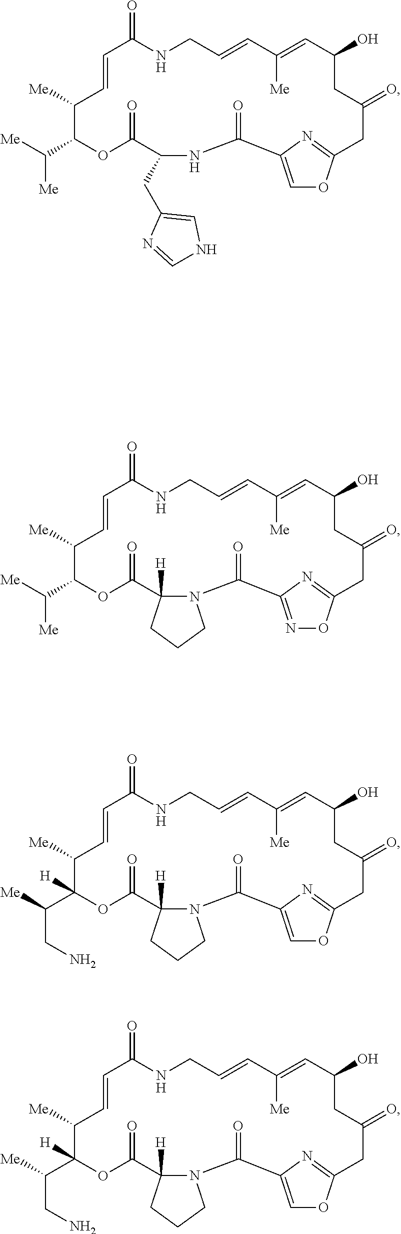 [SMILES: CC1=C\[C@@H](O)CC(=O)Cc2nc(co2)C(=O)N[C@H](Cc2c[nH]cn2)C(=O)O[C@H](C(C)C)[C@H](C)/C=C/C(=O)NC\C=C\1 ; CC1=C\[C@@H](O)CC(=O)Cc2nc(no2)C(=O)N2CCC[C@@H]2C(=O)O[C@H](C(C)C)[C@H](C)/C=C/C(=O)NC\C=C\1 ; CC1=C\[C@@H](O)CC(=O)Cc2nc(co2)C(=O)N2CCC[C@@H]2C(=O)O[C@H]([C@H](C)CN)[C@H](C)/C=C/C(=O)NC\C=C\1 ; CC1=C\[C@@H](O)CC(=O)Cc2nc(co2)C(=O)N2CCC[C@@H]2C(=O)O[C@H]([C@@H](C)CN)[C@H](C)/C=C/C(=O)NC\C=C\1]

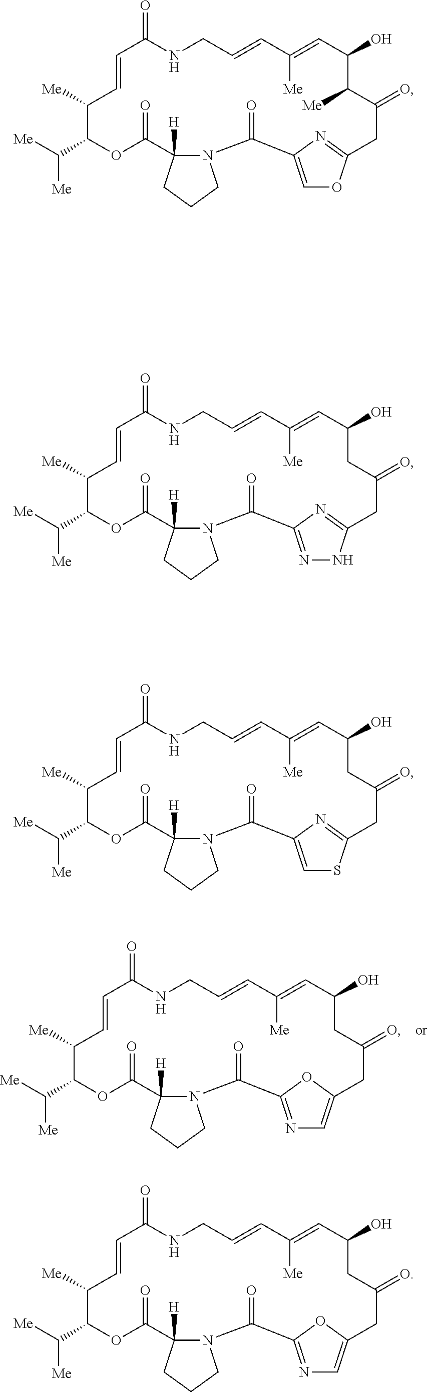 [SMILES: CC1=C\[C@@H](O)[C@H](C)C(=O)Cc2nc(co2)C(=O)N2CCC[C@@H]2C(=O)O[C@H](C(C)C)[C@H](C)/C=C/C(=O)NC\C=C\1 ; CC1=C\[C@@H](O)CC(=O)Cc2nc(n[nH]2)C(=O)N2CCC[C@@H]2C(=O)O[C@H](C(C)C)[C@H](C)/C=C/C(=O)NC\C=C\1 ; CC1=C\[C@@H](O)CC(=O)Cc2nc(cs2)C(=O)N2CCC[C@@H]2C(=O)O[C@H](C(C)C)[C@H](C)/C=C/C(=O)NC\C=C\1 ; CC1=C\[C@@H](O)CC(=O)Cc2cnc(o2)C(=O)N2CCC[C@@H]2C(=O)O[C@H](C(C)C)[C@H](C)/C=C/C(=O)NC\C=C\1 ; CC1=C\[C@@H](O)CC(=O)Cc2cnc(o2)C(=O)N2CCC[C@@H]2C(=O)O[C@H](C(C)C)[C@H](C)/C=C/C(=O)NC\C=C\1]

### Embodiment P54

A method of treating an infectious disease, said method comprising administering to a subject in need thereof an effective amount of a compound of embodiments P52 or 53.

### V. Additional Embodiments

### Embodiment 1

A compound, or salt thereof, having the formula:

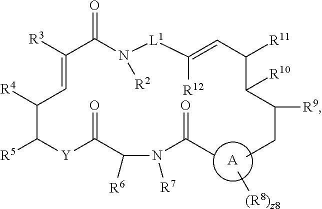

wherein
Y is —O— or —NH—;
L¹ is a bond, substituted or unsubstituted alkylene, or substituted or unsubstituted heteroalkylene;
R² is hydrogen or unsubstituted C₁-C₃ alkyl;
R³, R⁴, and R⁵ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —OPO₃H, —OSO₃H, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCH₂Cl, —OCH₂Br, —OCH₂I, —OCH₂F, —N₃, substituted or unsubstituted alkyl, substituted or unsubstituted heteroalkyl, or an amino acid side chain;
R⁷ is hydrogen, —CH₂COOH, —CONH₂, —OH, —SH, —NO₂, —NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶ and R⁷ may optionally be joined to form an unsubstituted heterocycloalkyl or unsubstituted heteroaryl;
R⁸ is oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Ring A is cycloalkylene, heterocycloalkylene, arylene, or heteroarylene;
z8 is an integer from 0 to 10;
R⁹ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ and R¹² are independently hydrogen, substituted or unsubstituted C₁-C₃ alkyl, or substituted or unsubstituted 2 to 3 membered heteroalkyl; and
R¹¹ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₂, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
wherein the compound does not have the formula:

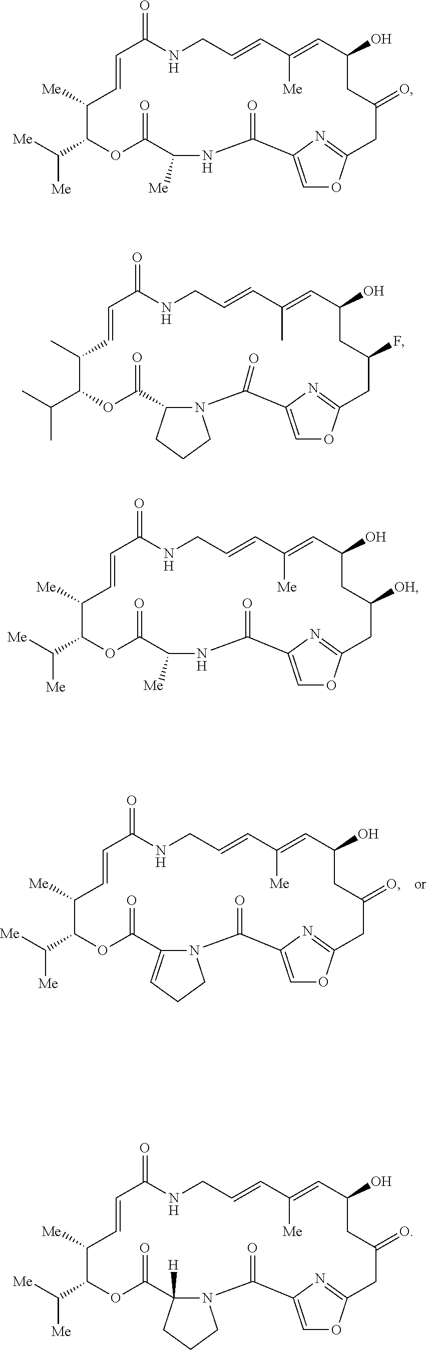 [SMILES: CC1=C\[C@@H](O)CC(=O)Cc2nc(co2)C(=O)N[C@H](C)C(=O)O[C@H](C(C)C)[C@H](C)/C=C/C(=O)NC\C=C\1 ; CC1=C\[C@@H](O)C[C@@H](F)Cc2nc(co2)C(=O)N2CCC[C@@H]2C(=O)O[C@H](C(C)C)[C@H](C)/C=C/C(=O)NC\C=C\1 ; CC1=C\[C@@H](O)C[C@@H](O)Cc2nc(co2)C(=O)N[C@H](C)C(=O)O[C@H](C(C)C)[C@H](C)/C=C/C(=O)NC\C=C\1 ; CC1=C\[C@@H](O)CC(=O)Cc2nc(co2)C(=O)N2CCC=C2C(=O)O[C@H](C(C)C)[C@H](C)/C=C/C(=O)NC\C=C\1 ; CC1=C\[C@@H](O)CC(=O)Cc2nc(co2)C(=O)N2CCC[C@@H]2C(=O)O[C@H](C(C)C)[C@H](C)/C=C/C(=O)NC\C=C\1]

### Embodiment 2

The compound of embodiment 1, wherein L¹ is a substituted or unsubstituted alkylene, substituted or unsubstituted heteroalkylene.

### Embodiment 3

The compound of embodiment 1, wherein L¹ is a substituted or unsubstituted alkenylene.

### Embodiment 4

The compound of embodiment 1, wherein L¹ is a substituted or unsubstituted C₁-C₃ alkenylene.

### Embodiment 5

The compound of any one of embodiments 1 to 4, wherein R² is hydrogen.

### Embodiment 6

The compound of any one of embodiments 1 to 5, wherein R³ is hydrogen or substituted or unsubstituted alkyl.

### Embodiment 7

The compound of any one of embodiments 1 to 5, wherein R³ is hydrogen.

### Embodiment 8

The compound of any one of embodiments 1 to 7, wherein R⁴ is hydrogen or substituted or unsubstituted alkyl.

### Embodiment 9

The compound of any one of embodiments 1 to 7, wherein R⁴ is hydrogen or a substituted or unsubstituted C₁-C₆ alkyl.

### Embodiment 10

The compound of any one of embodiments 1 to 9, wherein R⁵ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, or substituted or unsubstituted alkyl, or substituted or unsubstituted heteroalkyl.

### Embodiment 11

The compound of any one of embodiments 1 to 9, wherein R⁵ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment 12

The compound of any one of embodiments 1 to 9, wherein R⁵ is substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

### Embodiment 13

The compound of any one of embodiments 1 to 9, wherein R⁵ is substituted or unsubstituted C₁-C₈ alkyl.

### Embodiment 14

The compound of any one of embodiments 1 to 9, wherein R⁵ is substituted or unsubstituted C₁-C₃ alkyl.

### Embodiment 15

The compound of any one of embodiments 1 to 9, wherein R⁵ is:

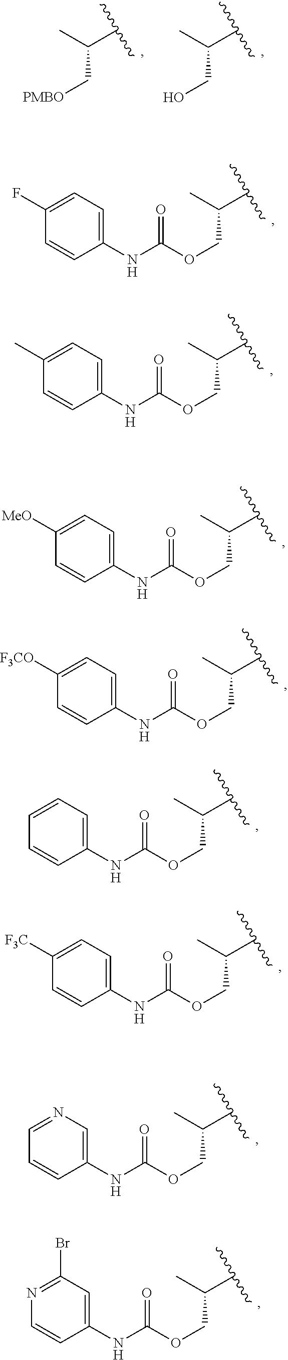

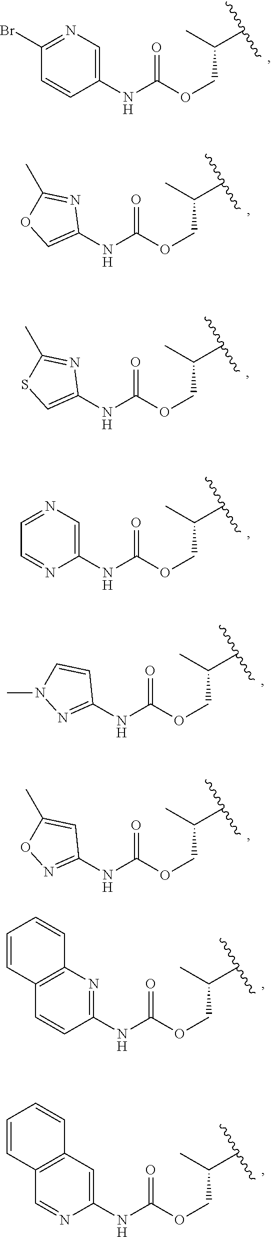

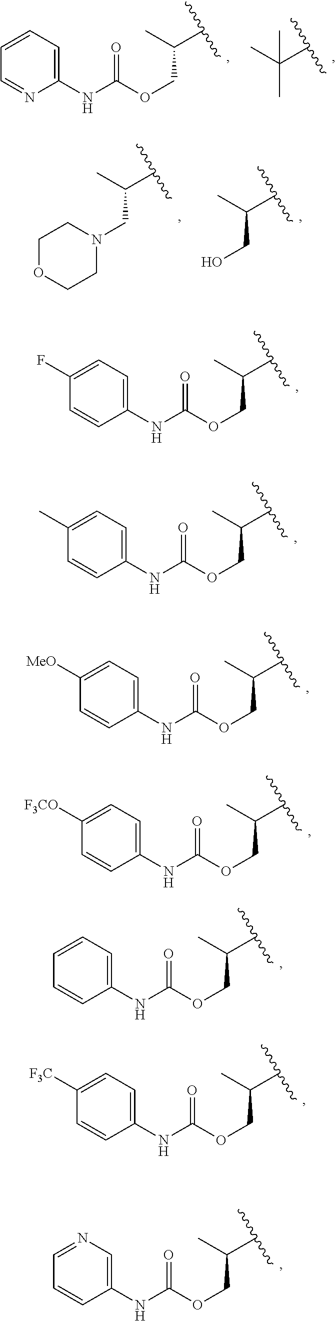

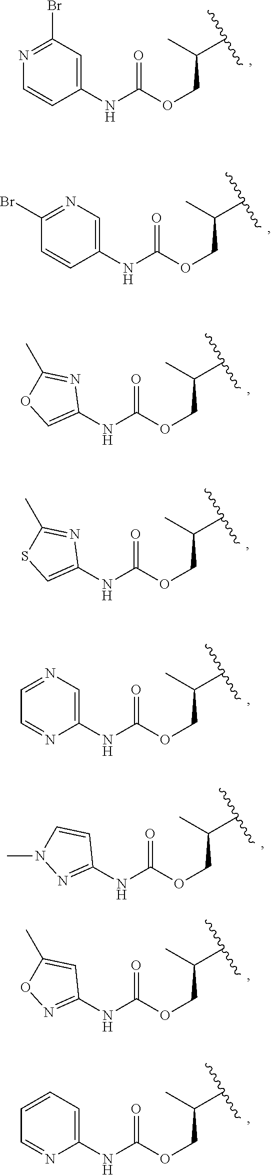

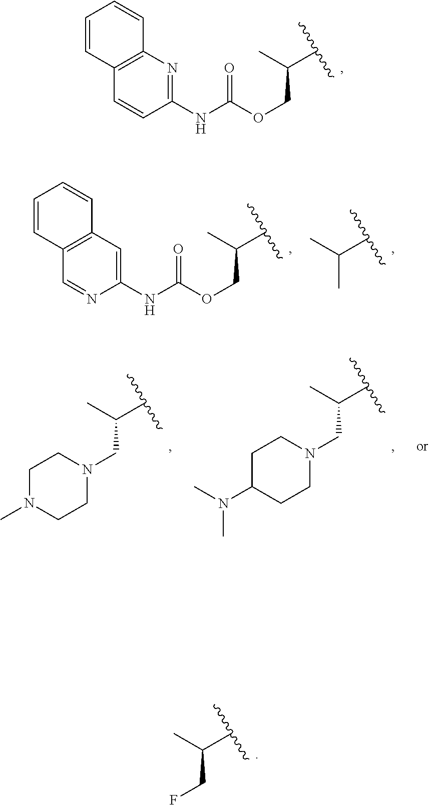

### Embodiment 17

The compound of any one of embodiments 1 to 16, wherein R⁶ is substituted or unsubstituted alkyl, substituted or unsubstituted heteroalkyl.

### Embodiment 18

The compound of any one of embodiments 1 to 16, wherein R⁶ is hydrogen,

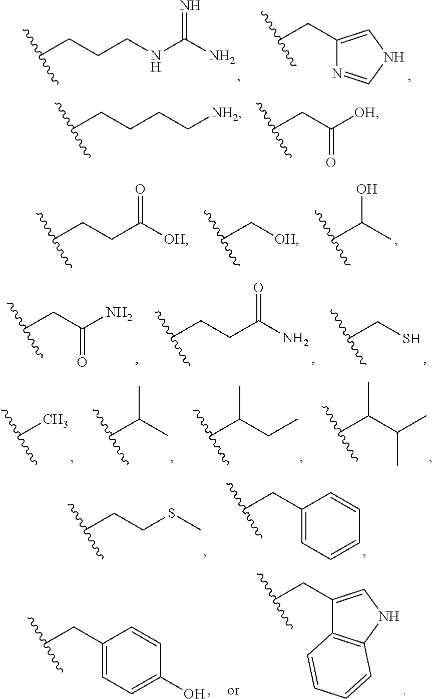

### Embodiment 19

The compound of any one of embodiments 1 to 16, wherein R⁶ is

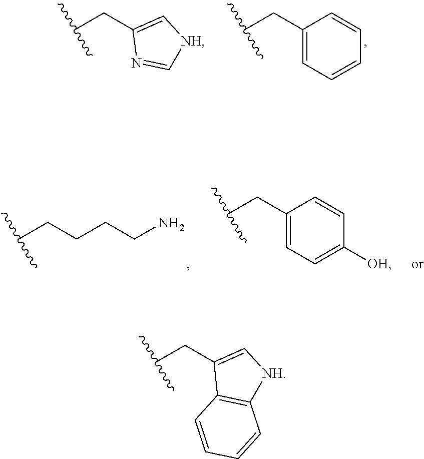 [SMILES: *Cc1c[nH]cn1 ; *Cc1ccccc1 ; *CCCCN ; *Cc1ccc(O)cc1 ; *Cc1c[nH]c2ccccc12]

### Embodiment 20

The compound of any one of embodiments 1 to 16, wherein R⁶ and R⁷ are joined to form an unsubstituted heterocycloalkyl or unsubstituted heteroaryl.

### Embodiment 21

The compound of any one of embodiments 1 to 16, wherein R⁶ and R⁷ are joined to form an unsubstituted 3 to 6 membered heterocycloalkyl, or an unsubstituted 5 to 6 membered heteroaryl.

### Embodiment 22

The compound of any one of embodiments 1 to 16, wherein R⁶ and R⁷ are joined to form an unsubstituted 3 to 6 membered heterocycloalkyl.

### Embodiment 23

The compound of any one of embodiments 1 to 16, wherein R⁶ and R⁷ are joined to form an unsubstituted pyrrolidinyl or 2,3-dihydropyrrolyl.

### Embodiment 24

The compound of any one of embodiments 1 to 19, wherein R⁷ is hydrogen, substituted or unsubstituted alkyl, or substituted or unsubstituted heteroalkyl.

### Embodiment 25

The compound of any one of embodiments 1 to 19, wherein R⁷ is hydrogen.

### Embodiment 26

The compound of any one of embodiments 1 to 25, wherein Ring A is C₃-C₆ cycloalkylene, 3 to 6 membered heterocycloalkylene, phenylene, or a 5 to 6 membered heteroaryl ene.

### Embodiment 27

The compound of any one of embodiments 1 to 25, wherein Ring A is 5 to 6 membered heteroarylene.

### Embodiment 28

The compound of any one of embodiments 1 to 25, wherein Ring A is imidazolylene, pyrrolylene, pyrazolylene, triazolylene, tetrazolylene, furanylene, oxazolylene, isooxazolylene, oxadiazolylene, oxatriazolylene, thienylene, thiazolylene, isothiazolylene, pyridinylene, pyrazinylene, pyrimidinylene, pyridazinylene, or triazinylene

### Embodiment 29

The compound of any one of embodiments 1 to 25, wherein Ring A is oxazolylene, thiazolylene, isooxazolylene, or oxadiazolylene.

### Embodiment 30

The compound of any one of embodiments 1 to 29, wherein z8 is O.

### Embodiment 31

The compound of any one of embodiments 1 to 30, wherein R⁹ is halogen, oxo, —NH₂, unsubstituted alkyl, or unsubstituted heteroalkyl.

### Embodiment 32

The compound of any one of embodiments 1 to 30, wherein R⁹ is —F, oxo, or —NH₂, or unsubstituted heteroalkyl.

### Embodiment 33

The compound of any one of embodiments 1 to 32, wherein R¹⁰ is hydrogen.

### Embodiment 34

The compound of any one of embodiments 1 to 33, wherein R¹¹ is —OH, —NH₂, or —SH.

### Embodiment 35

The compound of any one of embodiments 1 to 33, wherein R¹¹ is —OH.

### Embodiment 36

The compound of any one of embodiments 1 to 35, wherein R¹² is hydrogen or an unsubstituted C₁-C₃ alkyl.

### Embodiment 37

The compound of embodiment 1, having the formula:

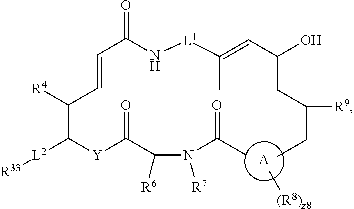

wherein
L² is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R³³ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCI₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment 38

The compound of embodiment 37, wherein -L²- is substituted or unsubstituted C₁-C₈ alkylene, substituted or unsubstituted 2 to 8 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroarylene.

### Embodiment 39

The compound of embodiment 37, wherein -L²- is substituted or unsubstituted C₁-C₈ alkylene, or substituted or unsubstituted 2 to 8 membered heteroalkylene.

### Embodiment 40

The compound of embodiment 37, wherein -L²- is

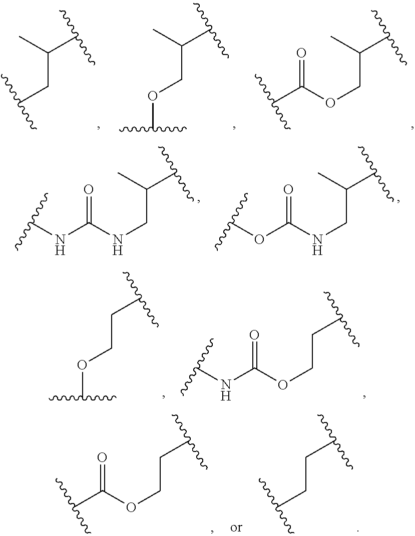

### Embodiment 41

The compound of embodiment 37, wherein -L²-R³³ is

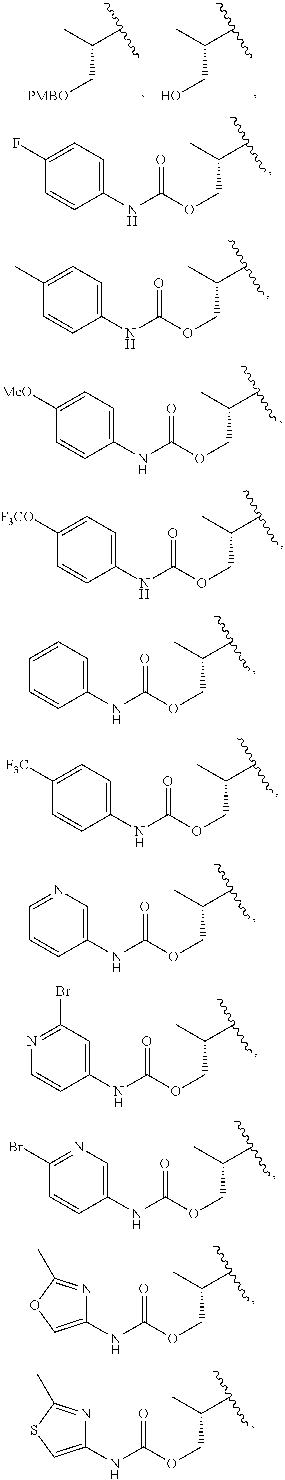

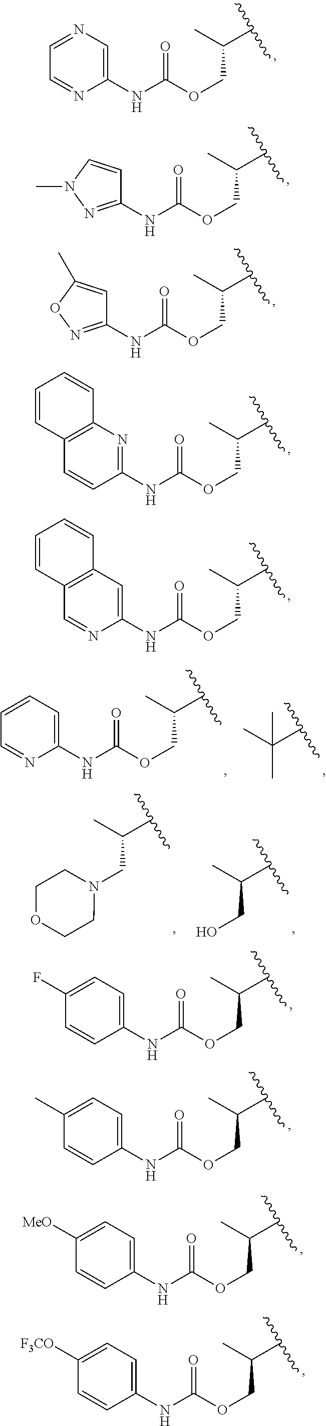

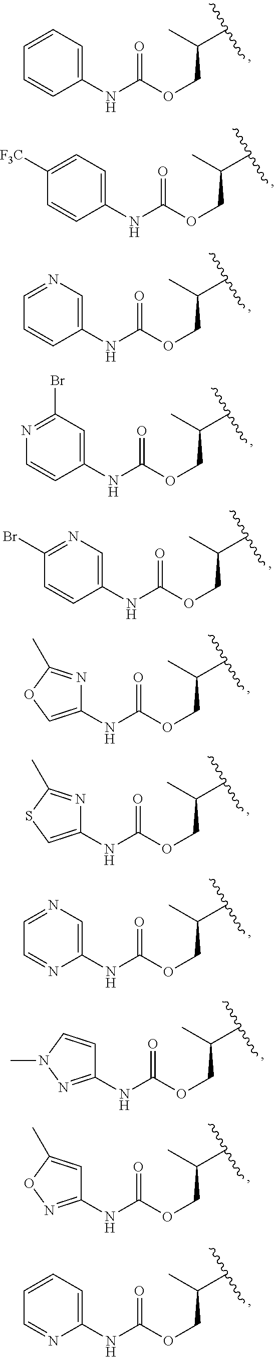

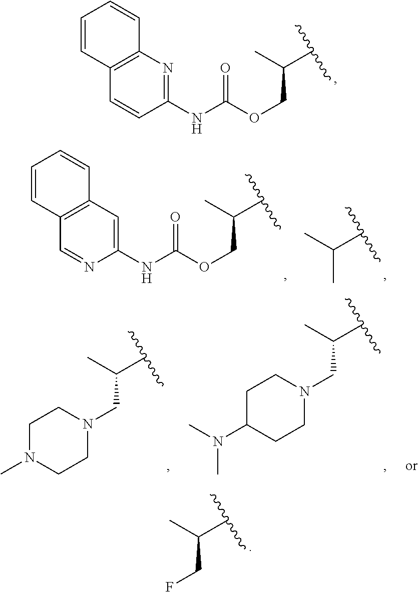

### Embodiment 42

The compound of embodiment 1, wherein the compound has the formula:

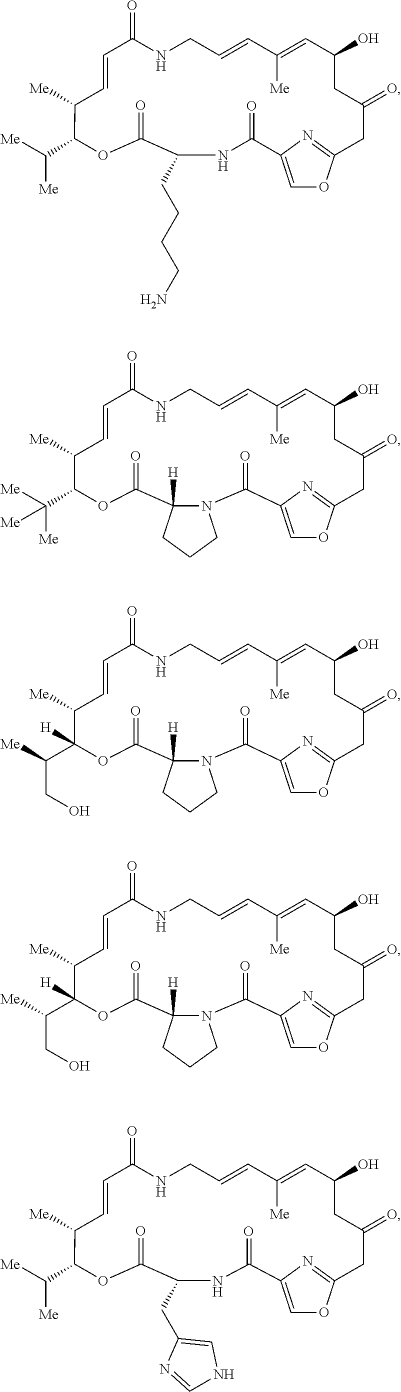 [SMILES: CC1=C\[C@@H](O)CC(=O)Cc2nc(co2)C(=O)N[C@H](CCCCN)C(=O)O[C@H](C(C)C)[C@H](C)/C=C/C(=O)NC\C=C\1 ; CC1=C\[C@@H](O)CC(=O)Cc2nc(co2)C(=O)N2CCC[C@@H]2C(=O)O[C@H](C(C)(C)C)[C@H](C)/C=C/C(=O)NC\C=C\1 ; CC1=C\[C@@H](O)CC(=O)Cc2nc(co2)C(=O)N2CCC[C@@H]2C(=O)O[C@H]([C@H](C)CO)[C@H](C)/C=C/C(=O)NC\C=C\1 ; CC1=C\[C@@H](O)CC(=O)Cc2nc(co2)C(=O)N2CCC[C@@H]2C(=O)O[C@H]([C@@H](C)CO)[C@H](C)/C=C/C(=O)NC\C=C\1 ; CC1=C\[C@@H](O)CC(=O)Cc2nc(co2)C(=O)N[C@H](Cc2c[nH]cn2)C(=O)O[C@H](C(C)C)[C@H](C)/C=C/C(=O)NC\C=C\1]

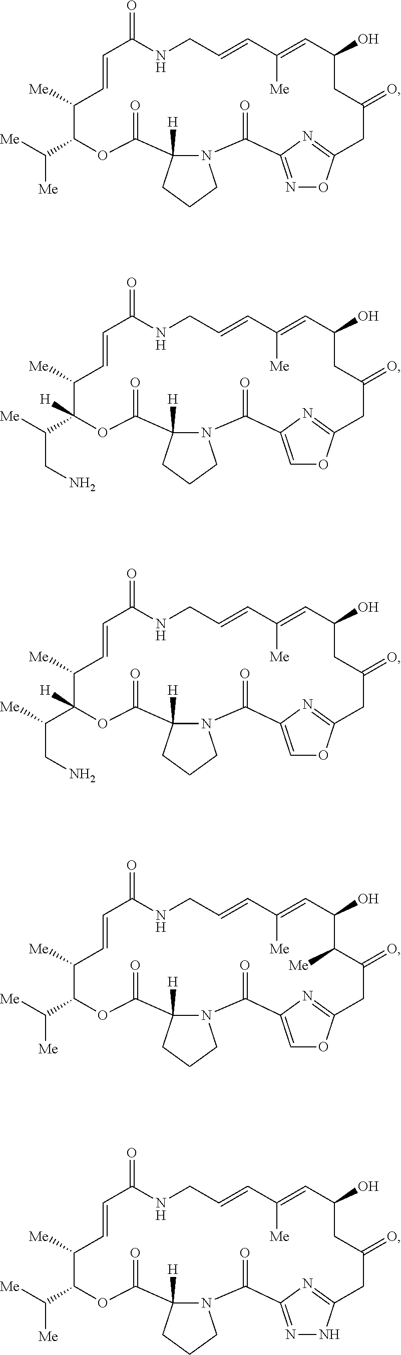 [SMILES: CC1=C\[C@@H](O)CC(=O)Cc2nc(no2)C(=O)N2CCC[C@@H]2C(=O)O[C@H](C(C)C)[C@H](C)/C=C/C(=O)NC\C=C\1 ; CC1=C\[C@@H](O)CC(=O)Cc2nc(co2)C(=O)N2CCC[C@@H]2C(=O)O[C@H](C(C)CN)[C@H](C)/C=C/C(=O)NC\C=C\1 ; CC1=C\[C@@H](O)CC(=O)Cc2nc(co2)C(=O)N2CCC[C@@H]2C(=O)O[C@H]([C@@H](C)CN)[C@H](C)/C=C/C(=O)NC\C=C\1 ; CC1=C\[C@@H](O)[C@H](C)C(=O)Cc2nc(co2)C(=O)N2CCC[C@@H]2C(=O)O[C@H](C(C)C)[C@H](C)/C=C/C(=O)NC\C=C\1 ; CC1=C\[C@@H](O)CC(=O)Cc2nc(n[nH]2)C(=O)N2CCC[C@@H]2C(=O)O[C@H](C(C)C)[C@H](C)/C=C/C(=O)NC\C=C\1]

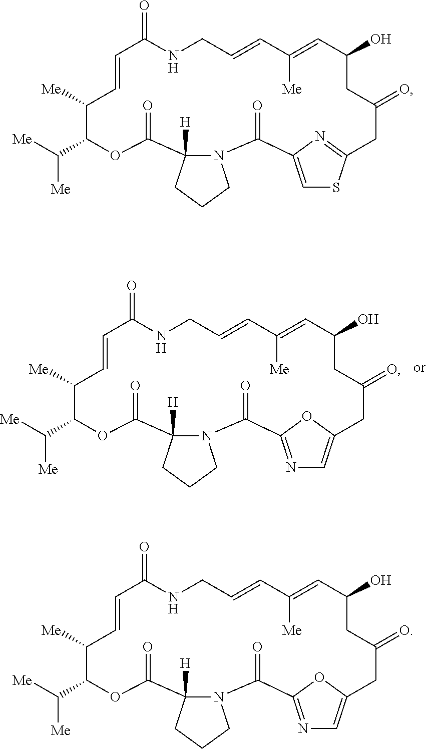 [SMILES: CC1=C\[C@@H](O)CC(=O)Cc2nc(cs2)C(=O)N2CCC[C@@H]2C(=O)O[C@H](C(C)C)[C@H](C)/C=C/C(=O)NC\C=C\1 ; CC1=C\[C@@H](O)CC(=O)CC2=CN=C(O2)C(=O)N2CCC[C@@H]2C(=O)O[C@H](C(C)C)[C@H](C)/C=C/C(=O)NC\C=C\1 ; CC1=C\[C@@H](O)CC(=O)CC2=CN=C(O2)C(=O)N2CCC[C@@H]2C(=O)O[C@H](C(C)C)[C@H](C)/C=C/C(=O)NC\C=C\1]

### Embodiment 43

The compound of embodiment 1, having the formula:

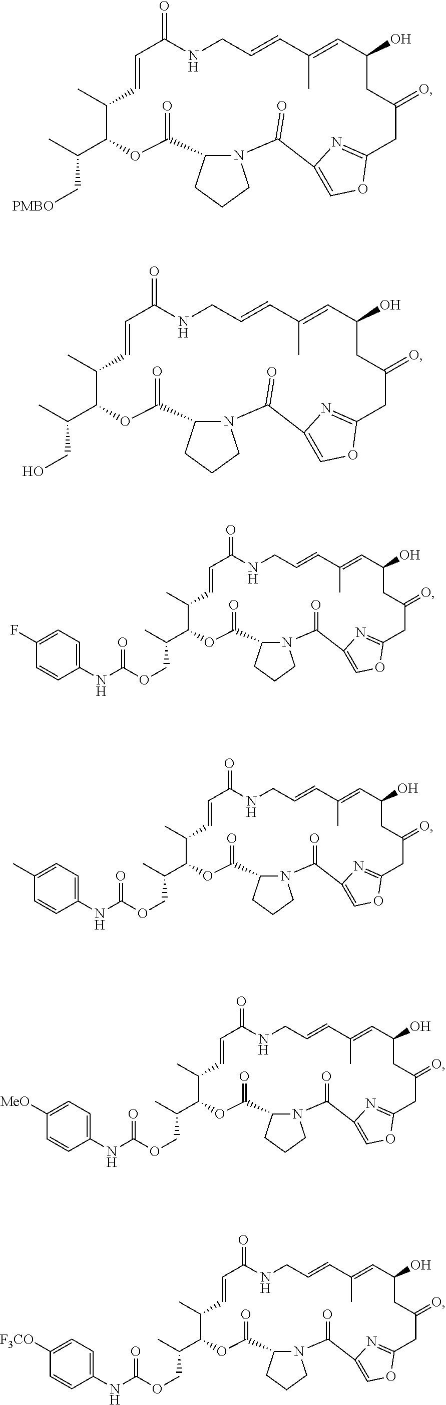

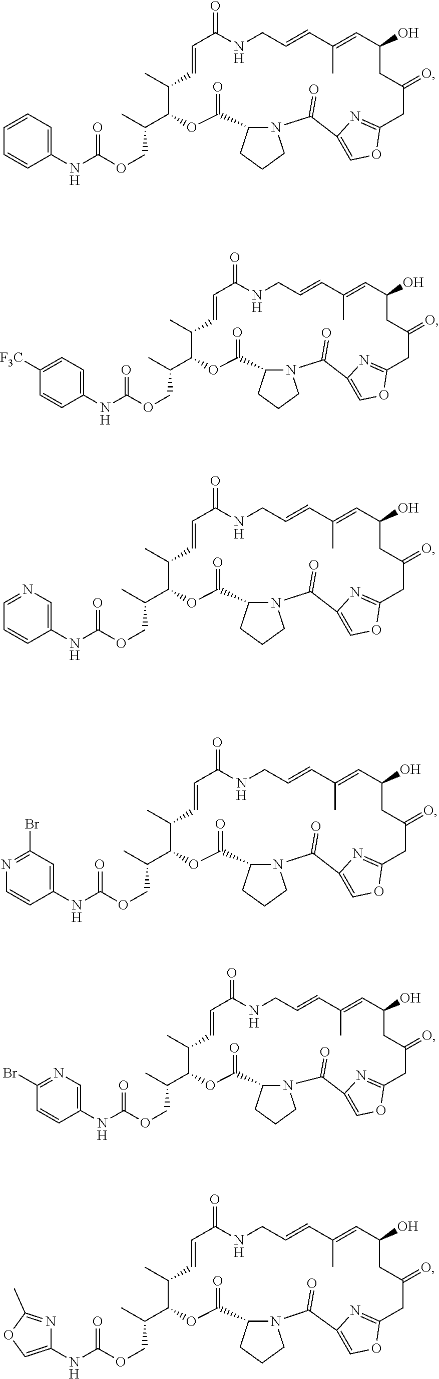

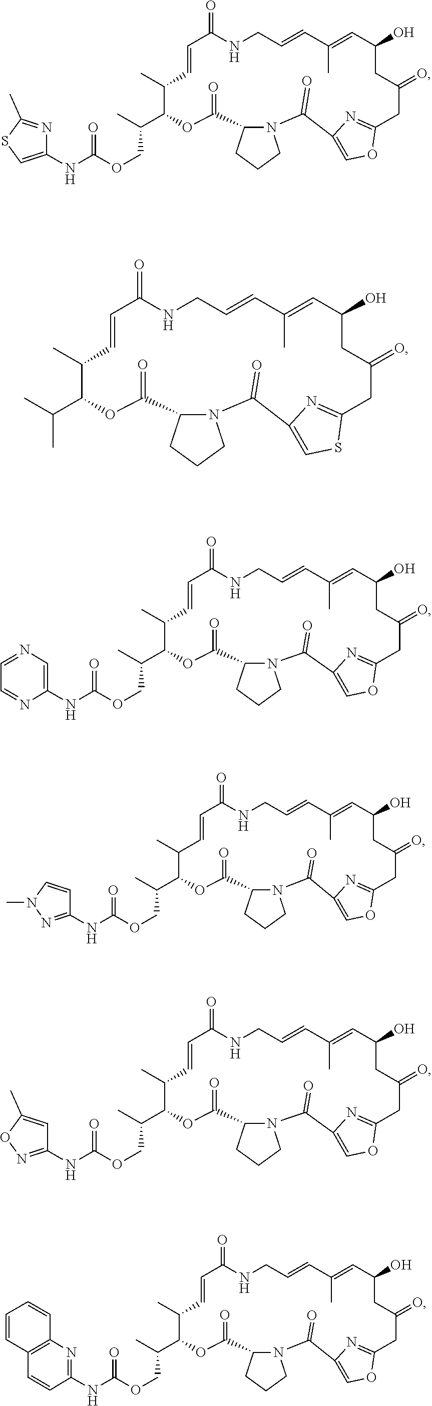

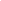

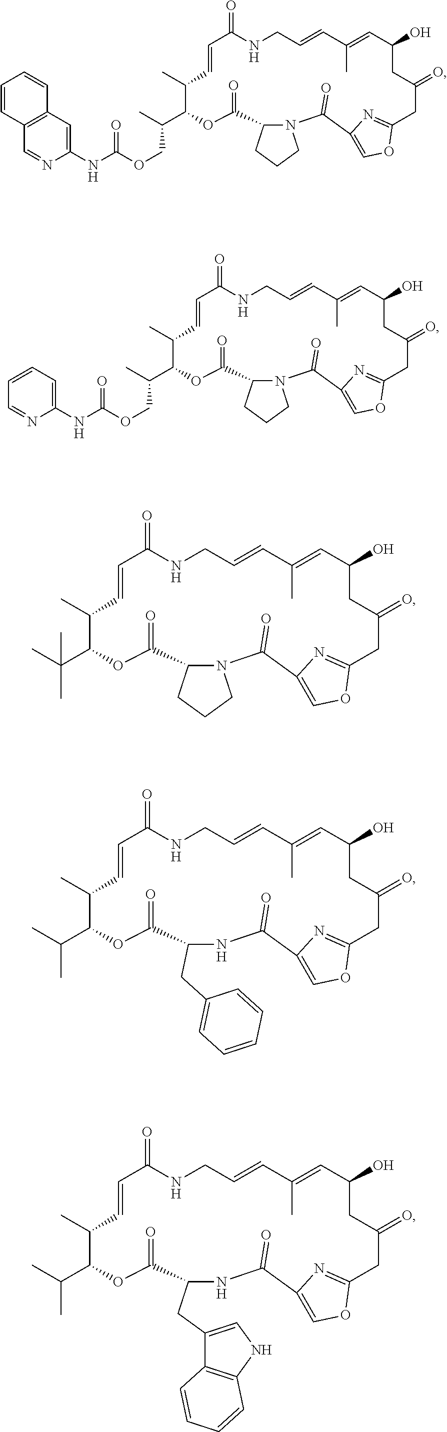

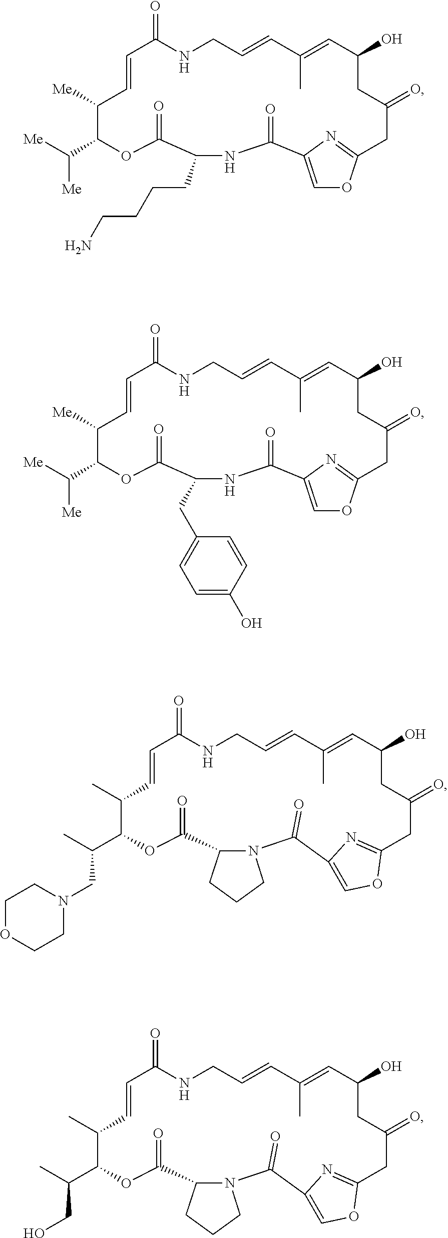 [SMILES: CC1=C\[C@@H](O)CC(=O)Cc2nc(co2)C(=O)N[C@H](CCCCN)C(=O)O[C@H](C(C)C)[C@H](C)/C=C/C(=O)NC\C=C\1 ; CC1=C\[C@@H](O)CC(=O)Cc2nc(co2)C(=O)N[C@H](Cc2ccc(O)cc2)C(=O)O[C@H](C(C)C)[C@H](C)/C=C/C(=O)NC\C=C\1 ; CC1=C\[C@@H](O)CC(=O)Cc2nc(co2)C(=O)N2CCC[C@@H]2C(=O)O[C@H]([C@@H](C)CN2CCOCC2)[C@H](C)/C=C/C(=O)NC\C=C\1 ; CC1=C\[C@@H](O)CC(=O)Cc2nc(co2)C(=O)N2CCC[C@@H]2C(=O)O[C@H]([C@H](C)CO)[C@H](C)/C=C/C(=O)NC\C=C\1]

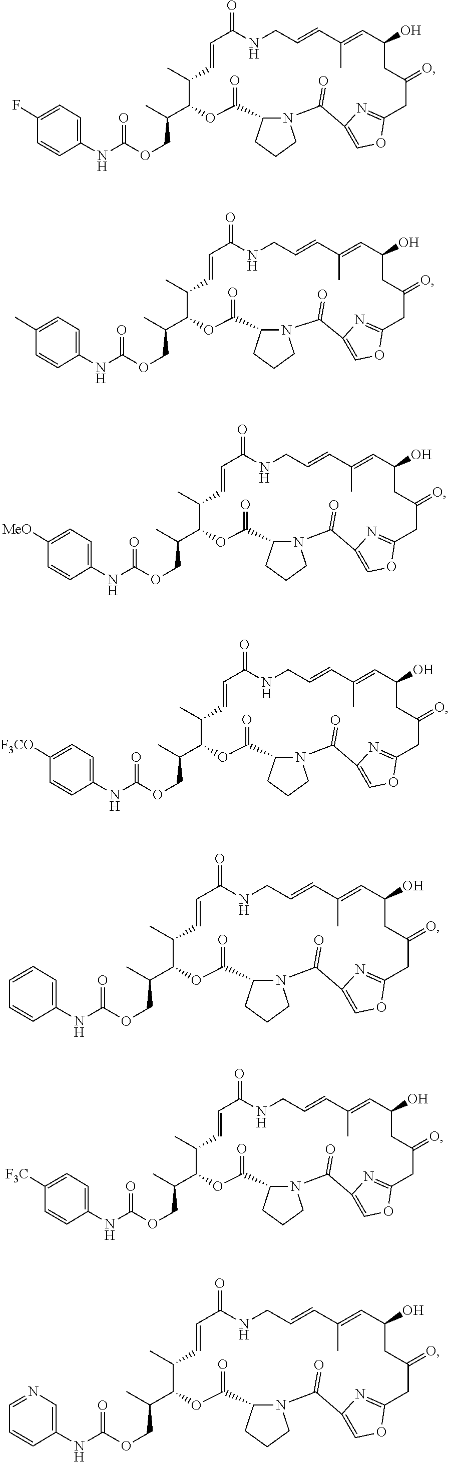

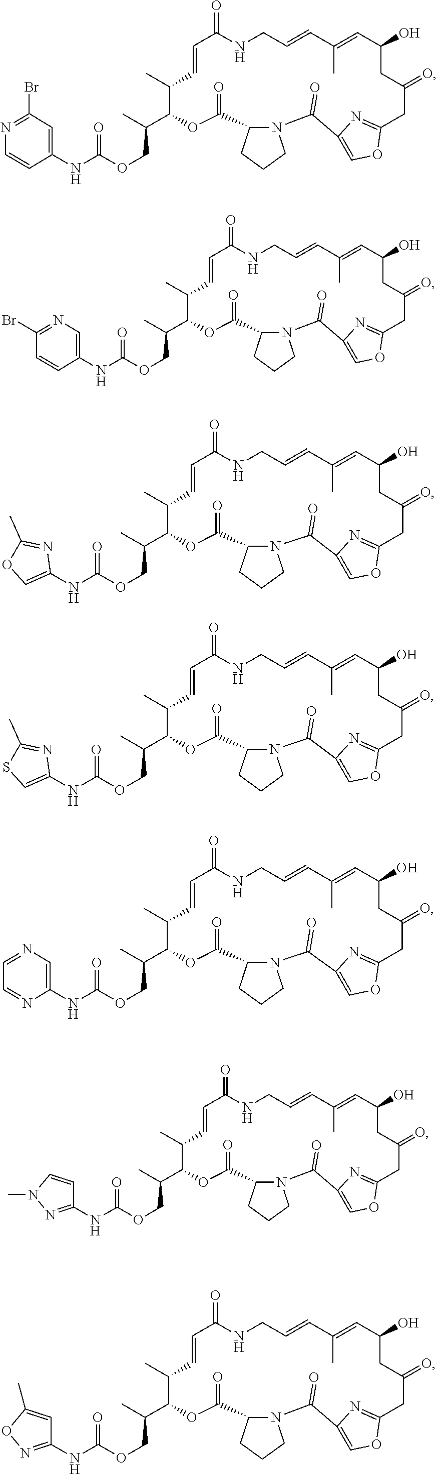

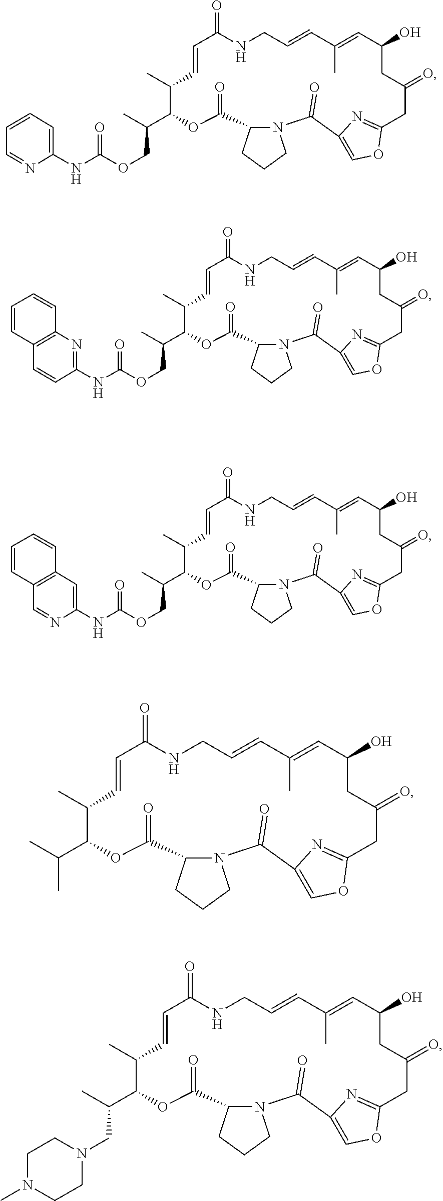

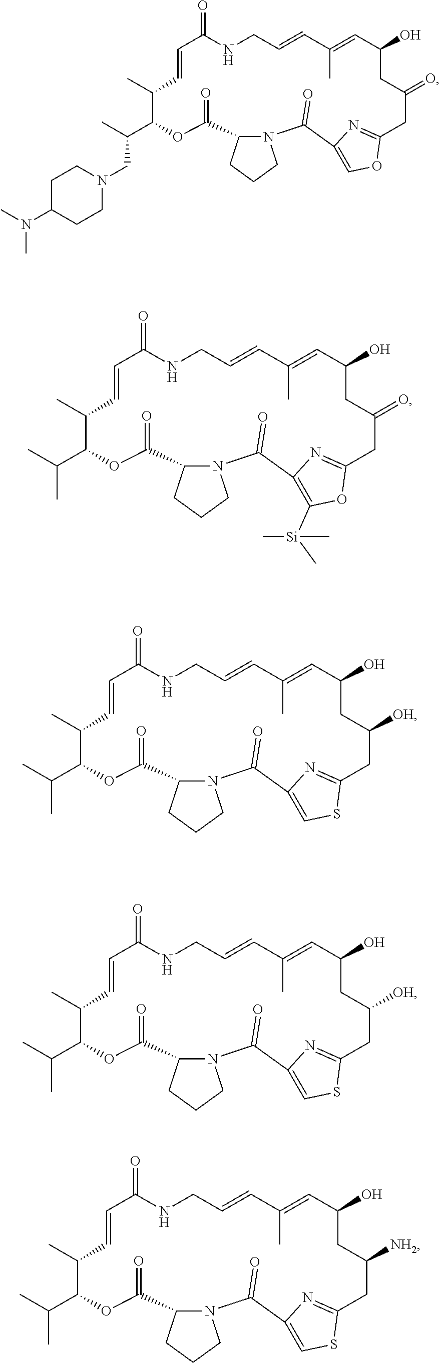

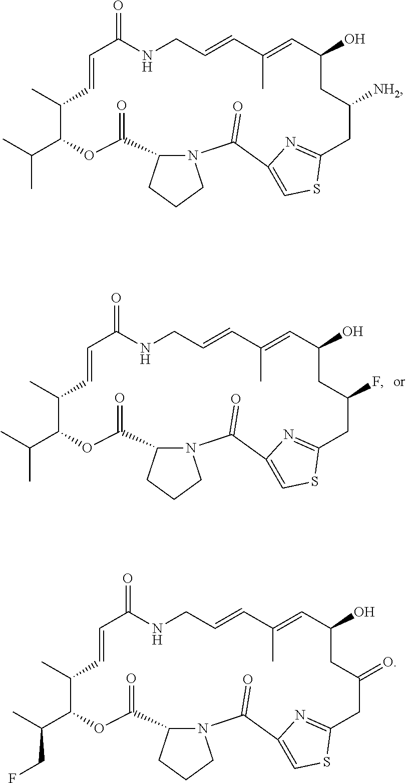 [SMILES: CC1=C\[C@@H](O)C[C@H](N)Cc2nc(cs2)C(=O)N2CCC[C@@H]2C(=O)O[C@H](C(C)C)[C@H](C)/C=C/C(=O)NC\C=C\1 ; CC1=C\[C@@H](O)C[C@@H](F)Cc2nc(cs2)C(=O)N2CCC[C@@H]2C(=O)O[C@H](C(C)C)[C@H](C)/C=C/C(=O)NC\C=C\1 ; CC1=C\[C@@H](O)CC(=O)Cc2nc(cs2)C(=O)N2CCC[C@@H]2C(=O)O[C@H]([C@H](C)CF)[C@H](C)/C=C/C(=O)NC\C=C\1]

### Embodiment 44

A compound, or salt thereof, having the formula:

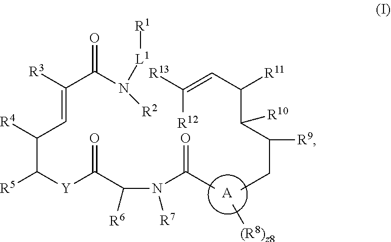

wherein
R¹ and R¹³ are independently a bioconjugate reactive moiety or a leaving group;
Y is —O— or —NH—;
L¹ is a bond, substituted or unsubstituted alkylene, or substituted or unsubstituted heteroalkylene;
R² is hydrogen or unsubstituted C₁-C₃ alkyl;
R³, R⁴, and R⁵ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —OPO₃H, —OSO₃H, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCH₂Cl, —OCH₂Br, —OCH₂I, —OCH₂F, —N₃, substituted or unsubstituted alkyl, substituted or unsubstituted heteroalkyl, or an amino acid side chain;
R⁷ is hydrogen, —CH₂COOH, —CONH₂, —OH, —SH, —NO₂, —NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ and R⁷ may optionally be joined to form a substituted or unsubstituted heterocycloalkyl or substituted or unsubstituted heteroaryl;
R⁸ is oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Ring A is cycloalkylene, heterocycloalkylene, arylene, or heteroarylene;
z8 is an integer from 0 to 10;
R⁹ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ and R¹² are independently hydrogen, substituted or unsubstituted C₁-C₃ alkyl, or substituted or unsubstituted 2 to 3 membered heteroalkyl; and
R¹¹ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment 45

A compound, or salt thereof, having the formula:

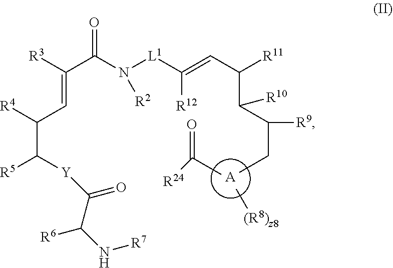

wherein
R²⁴ is a bioconjugate reactive moiety or leaving group;
Y is —O— or —NH—;
L¹ is a bond, substituted or unsubstituted alkylene, or substituted or unsubstituted heteroalkylene;
R² is hydrogen or unsubstituted C₁-C₃ alkyl;
R³, R⁴, and R⁵ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —OPO₃H, —OSO₃H, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCH₂Cl, —OCH₂Br, —OCH₂I, —OCH₂F, —N₃, substituted or unsubstituted alkyl, substituted or unsubstituted heteroalkyl, or an amino acid side chain;
R⁷ is hydrogen, —CH₂COOH, —CONH₂, —OH, —SH, —NO₂, —NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ and R⁷ may optionally be joined to form a substituted or unsubstituted heterocycloalkyl or substituted or unsubstituted heteroaryl;
R⁸ is oxo, halogen, —CCl₂, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Ring A is cycloalkylene, heterocycloalkylene, arylene, or heteroarylene;
z8 is an integer from 0 to 10;
R⁹ is hydrogen, oxo, halogen, —CCl₂, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ and R¹² are independently hydrogen, substituted or unsubstituted C₁-C₃ alkyl, or substituted or unsubstituted 2 to 3 membered heteroalkyl; and
R¹¹ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment 46

A compound, or salt thereof, having the formula:

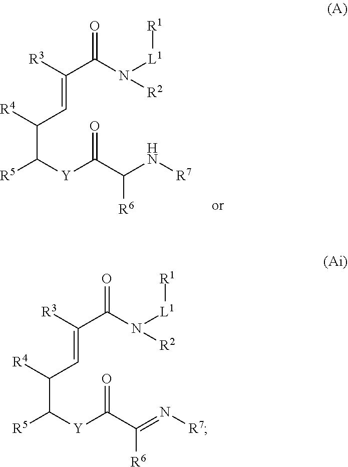

wherein
R¹ is a bioconjugate reactive moiety or leaving group;
Y is —O— or —NH—;
L¹ is a bond, substituted or unsubstituted alkylene, or substituted or unsubstituted heteroalkylene;
R² is hydrogen or unsubstituted C₁-C₃ alkyl;
R³, R⁴ and R⁵ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —OPO₃H, —OSO₃H, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCH₂Cl, —OCH₂Br, —OCH₂I, —OCH₂F, —N₃, substituted or unsubstituted alkyl, substituted or unsubstituted heteroalkyl, or an amino acid side chain;
R⁷ is hydrogen, —CH₂COOH, —CONH₂, —OH, —SH, —NO₂, —NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R⁶ and R⁷ may optionally be joined to form a substituted or unsubstituted heterocycloalkyl or substituted or unsubstituted heteroaryl.

### Embodiment 47

A compound, or salt thereof, having the formula:

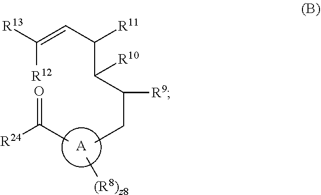

wherein
R¹³ and R²⁴ are independently bioconjugate reactive moieties or leaving groups;
R⁸ is oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Ring A is cycloalkylene, heterocycloalkylene, arylene, or heteroarylene;
z8 is an integer from 0 to 10;
R⁹ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ and R¹² are independently hydrogen, substituted or unsubstituted C₁-C₃ alkyl, or substituted or unsubstituted 2 to 3 membered heteroalkyl; and
R¹¹ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment 48

The compound of embodiment 47, wherein compound B has the formula:

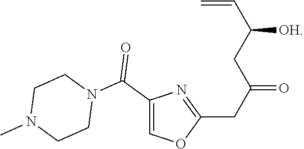 [SMILES: C=C[C@@H](O)CC(=O)Cc1nc(C(=O)N2CCN(C)CC2)co1]

### Embodiment 49

A method of making a compound, or salt thereof, having the formula:

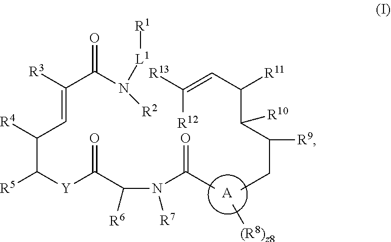

said method comprising mixing compound A and compound B together in a reaction vessel; wherein compound A has the formula:

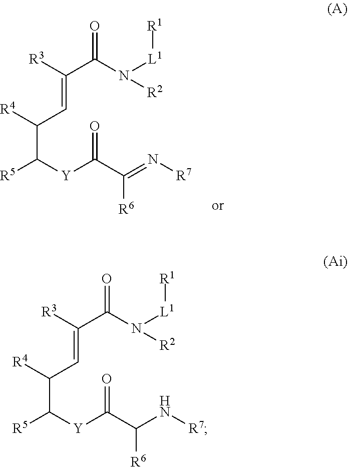

and
compound B has the formula:

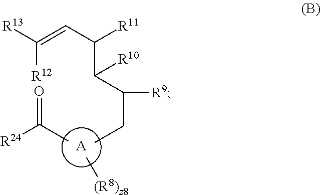

wherein
R¹, R¹³, and R²⁴ are independently bioconjugate reactive moieties or leaving groups;
Y is —O— or —NH—;
L¹ is a bond, substituted or unsubstituted alkylene, or substituted or unsubstituted heteroalkylene;
R² is hydrogen or unsubstituted C₁-C₃ alkyl;
R³, R⁴, and R⁵ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —OPO₃H, —OSO₃H, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCH₂Cl, —OCH₂Br, —OCH₂I, —OCH₂F, —N₃, substituted or unsubstituted alkyl, substituted or unsubstituted heteroalkyl, or an amino acid side chain;
R⁷ is hydrogen, —CH₂COOH, —CONH₂, —OH, —SH, —NO₂, —NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ and R⁷ may optionally be joined to form a substituted or unsubstituted heterocycloalkyl or substituted or unsubstituted heteroaryl;
R⁸ is oxo, halogen, —CCl₂, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Ring A is cycloalkylene, heterocycloalkylene, arylene, or heteroarylene;
z8 is an integer from 0 to 10;
R⁹ is hydrogen, oxo, halogen, —CCl₂, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ and R¹² are independently hydrogen, substituted or unsubstituted C₁-C₃ alkyl, or substituted or unsubstituted 2 to 3 membered heteroalkyl;
R¹¹ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment 50

A method of making a compound, or salt thereof, having the formula:

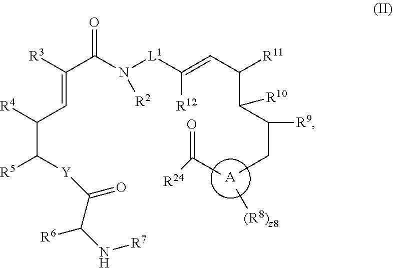

said method comprising mixing compound A and compound B together in a reaction vessel; wherein compound A has the formula:

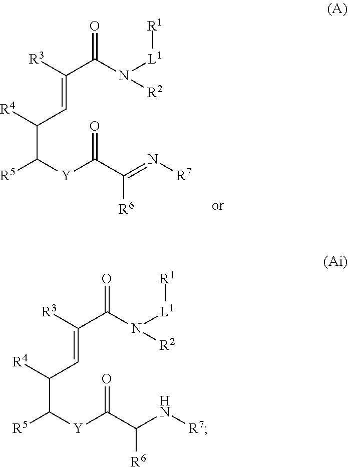

and
compound B has the formula:

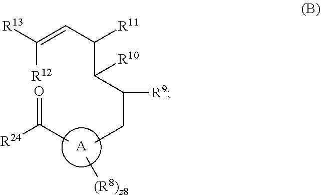

wherein
R¹, R¹³, and R²⁴ are independently bioconjugate reactive moieties or leaving groups;
Y is —O— or —NH—;
L¹ is a bond, substituted or unsubstituted alkylene, or substituted or unsubstituted heteroalkylene;
R² is hydrogen or unsubstituted C₁-C₃ alkyl;
R³, R⁴, and R⁵ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —OPO₃H, —OSO₃H, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCH₂Cl, —OCH₂Br, —OCH₂I, —OCH₂F, —N₃, substituted or unsubstituted alkyl, substituted or unsubstituted heteroalkyl, or an amino acid side chain;
R⁷ is hydrogen, —CH₂COOH, —CONH₂, —OH, —SH, —NO₂, —NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ and R⁷ may optionally be joined to form a substituted or unsubstituted heterocycloalkyl or substituted or unsubstituted heteroaryl;
R⁸ is oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Ring A is cycloalkylene, heterocycloalkylene, arylene, or heteroarylene;
z8 is an integer from 0 to 10;
R⁹ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ and R¹² are independently hydrogen, substituted or unsubstituted C₁-C₃ alkyl, or substituted or unsubstituted 2 to 3 membered heteroalkyl;
R¹¹ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment 51

The method of embodiment 49 or 50, further comprising exposing the compound of Formula (I) or Formula (II) to conditions conducive to form compound D, wherein compound D has the formula:

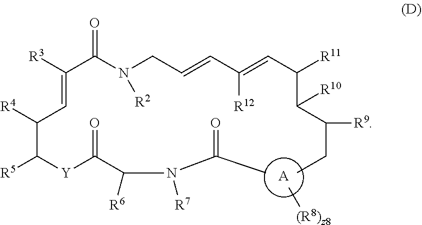

### Embodiment 52

The method of embodiment 49 or 50, wherein R¹ is an organotin heteroalkyl.

### Embodiment 53

The method of embodiment 49 or 50, wherein R¹ is

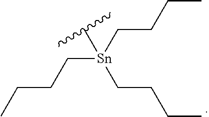 [SMILES: [*][Sn]([CH2]CCC)([CH2]CCC)[CH2]CCC]

### Embodiment 54

The method of embodiment 49 or 50, wherein R¹ and R¹³ are C₁-C₃ alkenylene.

### Embodiment 55

The method of embodiment 49 or 50, wherein R¹ and R¹³ react to form a bioconjugate linker.

### Embodiment 56

The method of embodiment 49 or 50, wherein compound A has the formula:

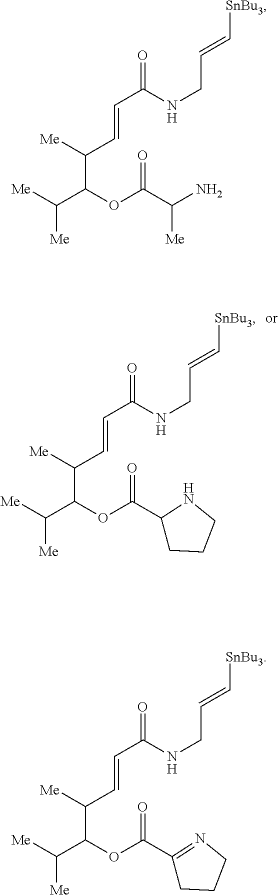

### Embodiment 57

The method of embodiment 49 or 50, wherein compound B has the formula:

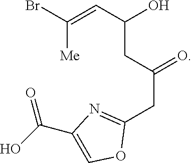 [SMILES: C/C(Br)=C\C(O)CC(=O)Cc1nc(C(=O)O)co1]

### Embodiment 58

The method of embodiment 49 or 50, further comprising exposing the compound of Formula (I) or Formula (II) to conditions conducive to form compound D, wherein compound D has the formula:

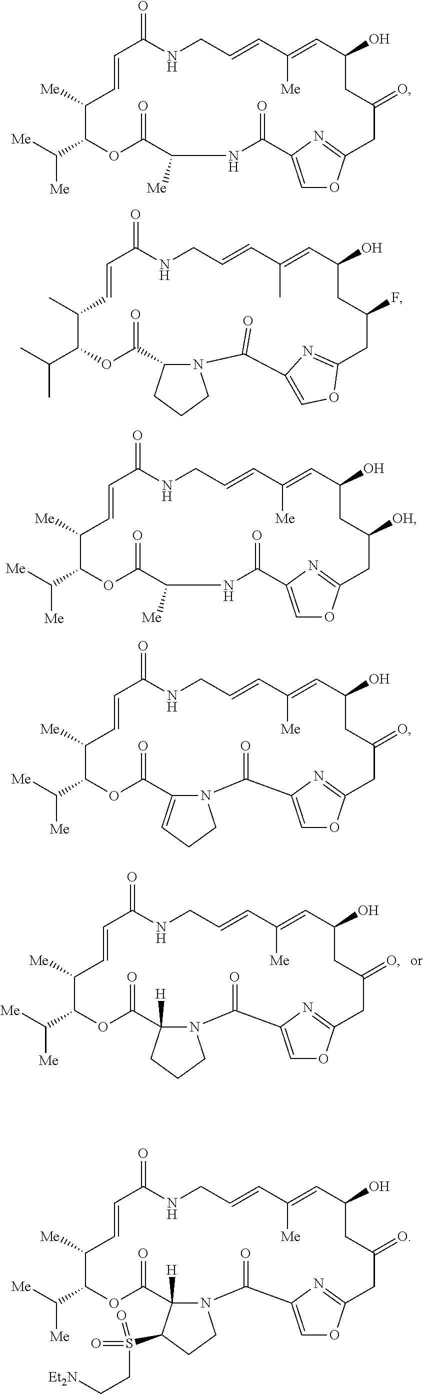

### Embodiment 59

The method of embodiment 49 or 50, wherein compound A is made by mixing compound A1 and compound A2 in a reaction vessel; wherein
compound A1 has the formula:

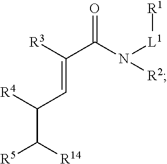

and
compound A2 has the formula:

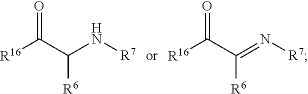

wherein
R¹⁴ and R¹⁶ are leaving groups.

### Embodiment 60

The method of embodiment 59, wherein compound A1 is made by mixing compound A3, compound A4, and compound A5 in a reaction vessel; wherein
compound A3 has the formula:

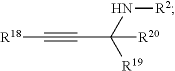

compound A4 has the formula:

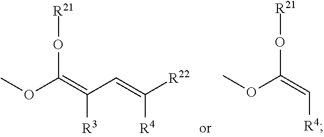

compound A5 has the formula:

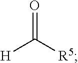

wherein
R¹⁸, R¹⁹, R²⁰, and R²² are independently hydrogen, substituted or unsubstituted alkyl, substituted or unsubstituted heteroalkyl; and
R²¹ is hydrogen or a protecting group.

### Embodiment 61

The method of embodiment 49 or 50, wherein compound B is made by mixing compound B1 and compound B2; wherein
compound B1 has the formula:

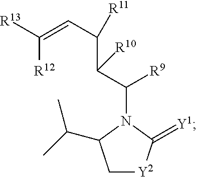

and
compound B2 has the formula:

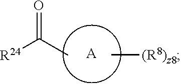

wherein
Y¹ is ═O or S;
Y² is —O— or —S—; and
R²⁴ is a bioconjugate reactive moiety or a leaving group.

### Embodiment 62

The method of embodiment 61, wherein compound B2 has the formula:

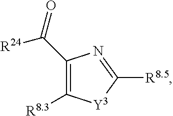

wherein
Y³ is —S—, —O—, or —NR^{8.4}-;
R^{8.4} is hydrogen, halogen, —CCl₂, —CBr₃, —CF₃, —CI₃, CHCl₂, —CHBr₂, —CHF₂, —CHI₂, —CH₂Cl, —CH₂Br, —CH₂F, —CH₂I, —CN, —CCl₃, —COOH, —CH₂COOH, —CONH₂, —OH, —SH, substituted or unsubstituted alkyl, or substituted or unsubstituted heteroalkyl; and
R^{8.3} and R^{8.5} are independently hydrogen, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, or substituted or unsubstituted heteroalkyl.

### Embodiment 63

The method of embodiment 61, wherein compound B1 is made by mixing compound B3 and compound B4, wherein
compound B3 has the formula:

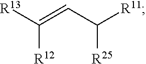

compound B4 has the formula:

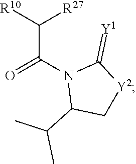

wherein
Y¹ is ═O or S;
Y² is —O— or —S—; and
R²⁵ and R²⁷ are leaving groups.

### Embodiment 64

A pharmaceutical composition comprising compound of embodiments 1 to 43, or pharmaceutically acceptable salt thereof, and a pharmaceutically acceptable excipient.

### Embodiment 65

A method of treating an infectious disease, said method comprising administering to a subject in need thereof an effective amount of a compound of embodiments 1 to 43.

### Embodiment 66

The method of embodiment 65, wherein said infection disease is a bacterial infection.

### Embodiment 67

The method of embodiment 65 or 66, wherein the infectious disease is Gram-positive bacterial infection.

### Embodiment 68

The method of embodiment 65 or 66, wherein the infectious disease is Gram-negative bacterial infection.

### Embodiment 69

The method of embodiment 65 or 66, wherein the bacterial infection is a Staphylococcus infection, an Enterococcus infection, an Acinetobacter infection, a Bacillus infection, a Streptococcus infection, an Escherichia infection, a Pseudomonas infection, a Klebsiella infection, or a Haemophilus infection.

### Embodiment 70

The method of embodiment 65 or 66, wherein the bacterial infection is a Staphylococcus infection, a Bacillus infection, a Streptococcus infection, an Escherichia infection, or a Haemophilus infection.

### EXAMPLES

### Example 1: Modular, Scalable Syntheses of Group a Streptogramin Compositions

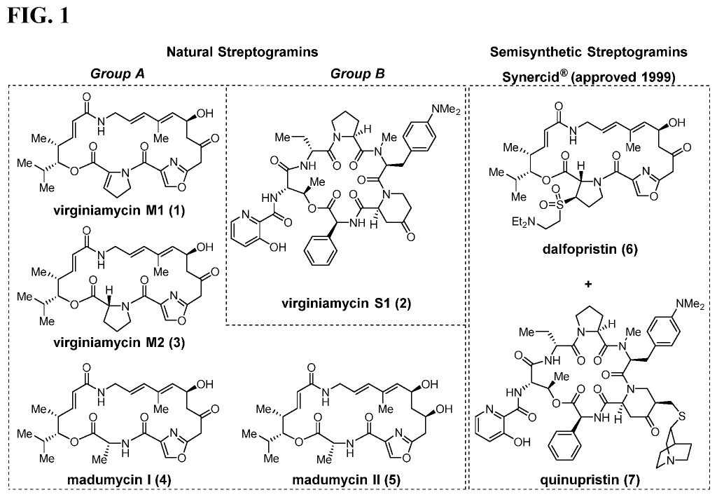

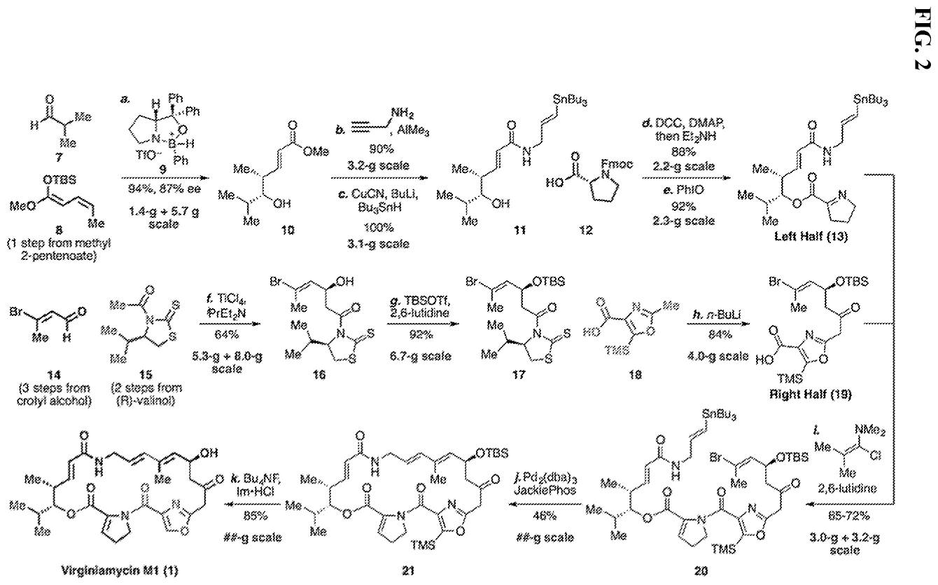

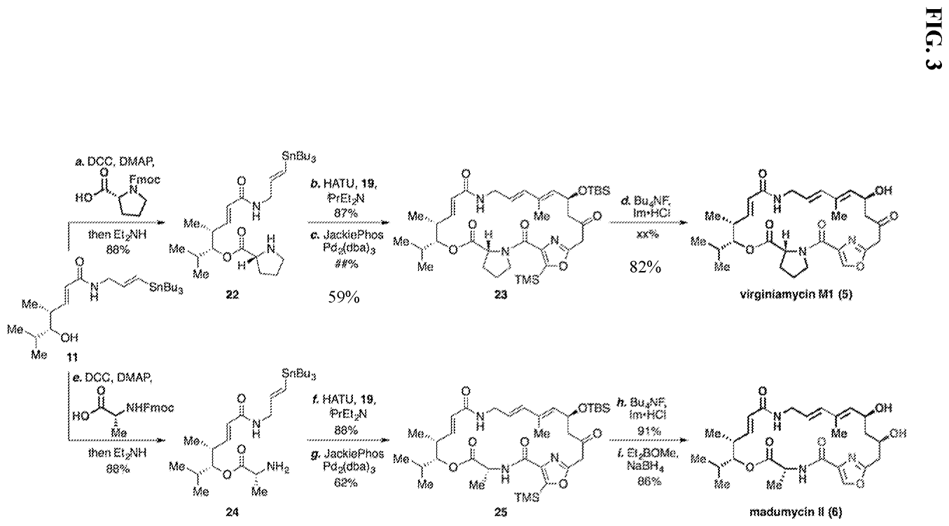

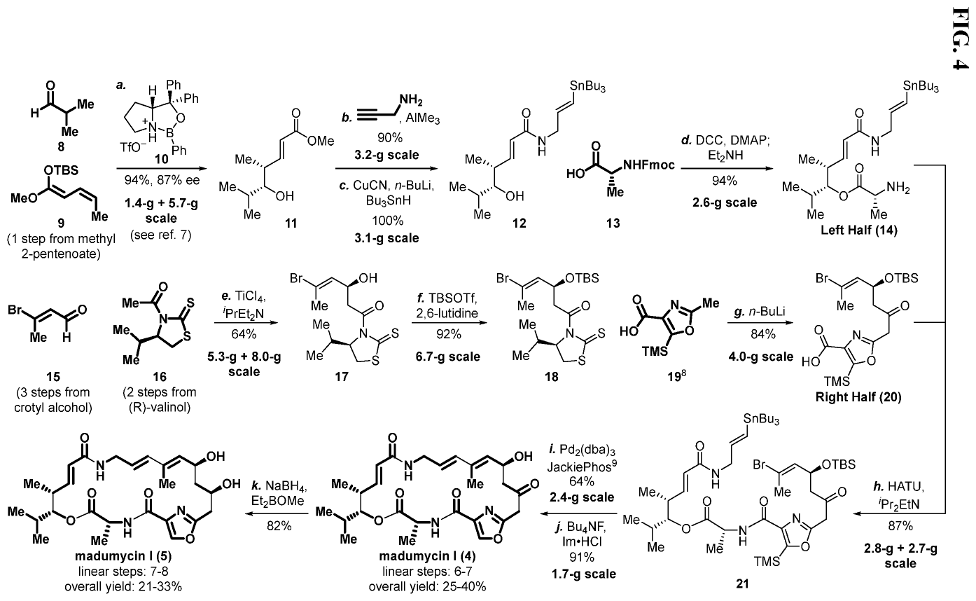

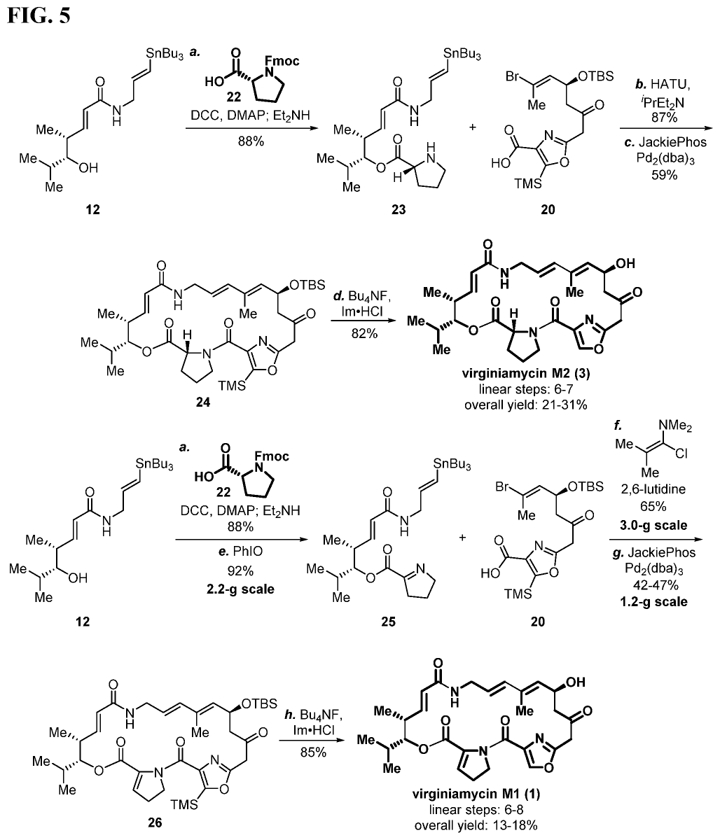

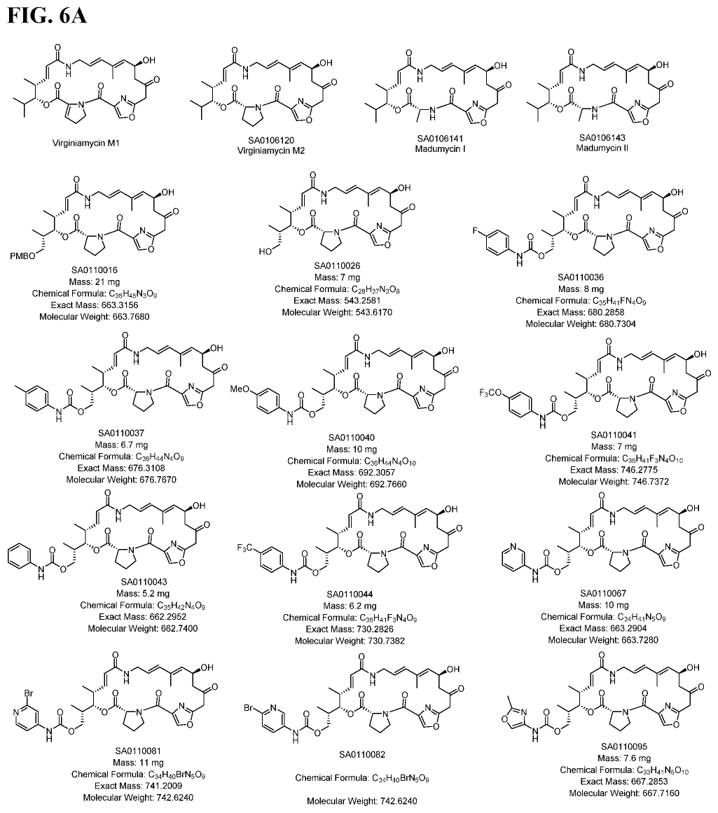

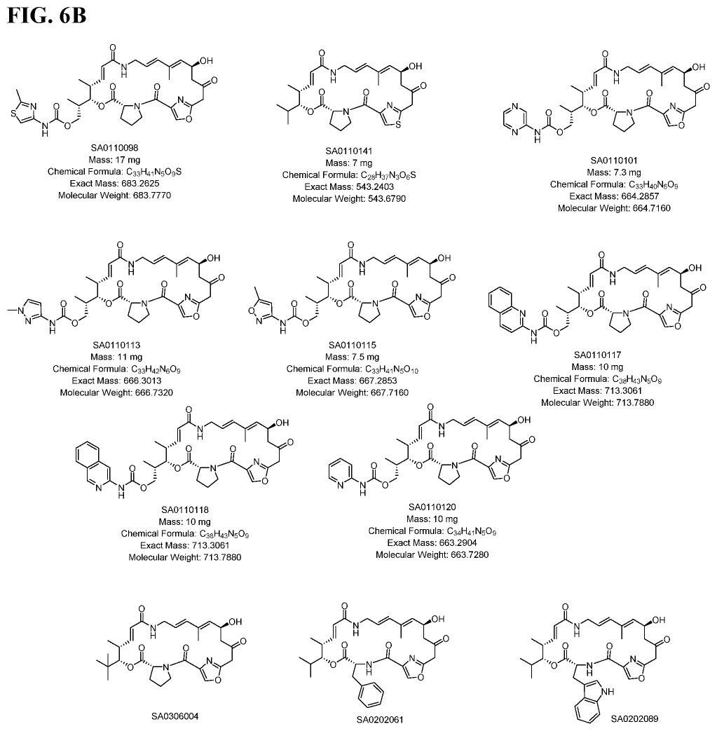

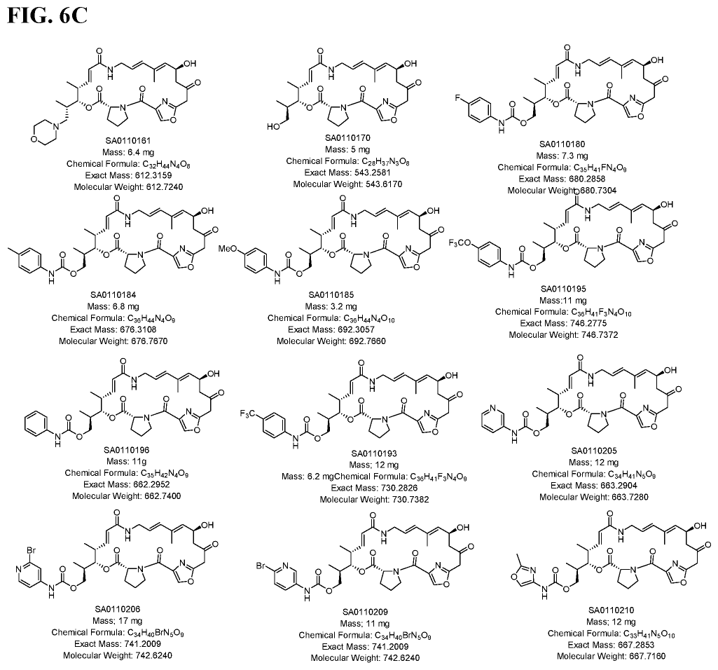

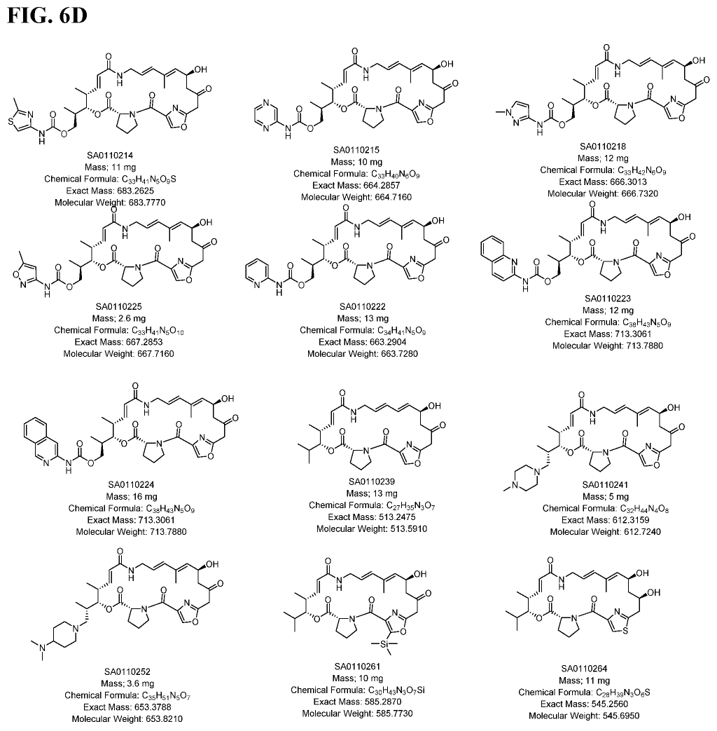

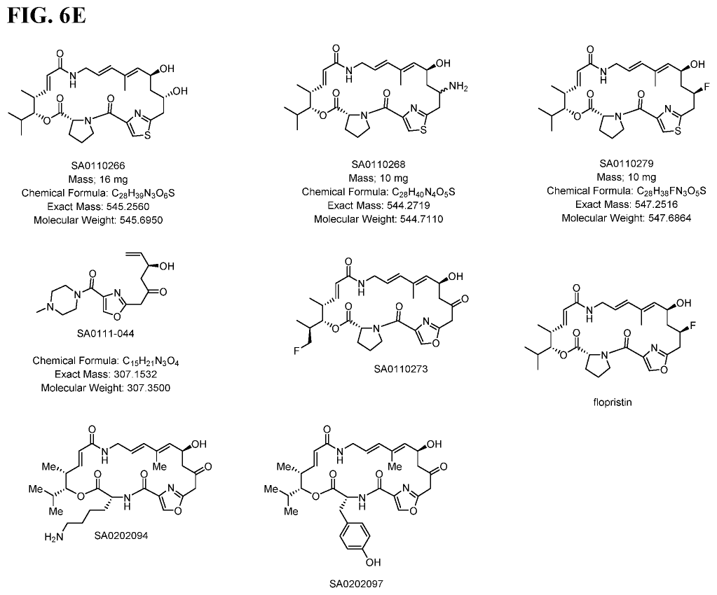

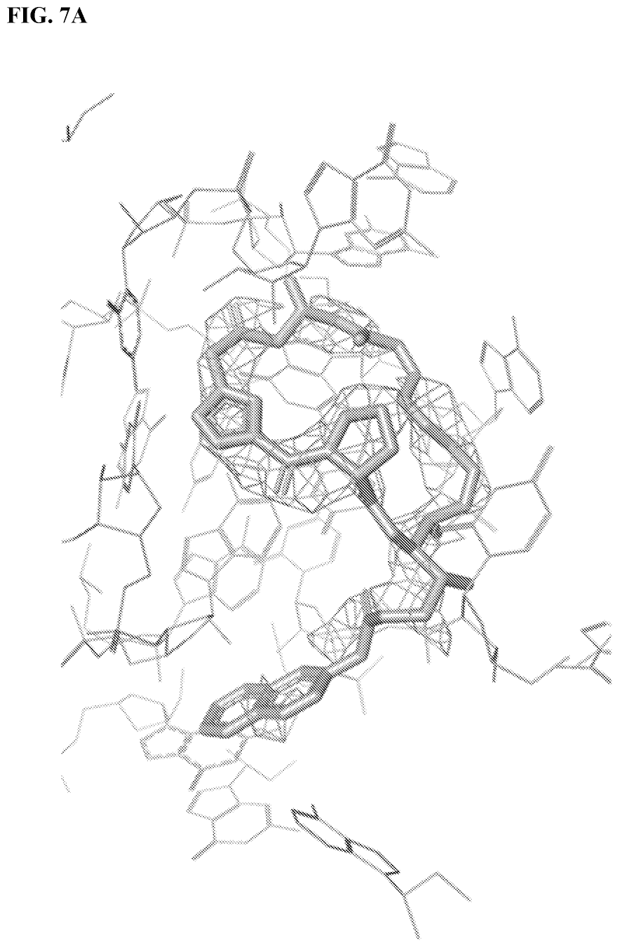

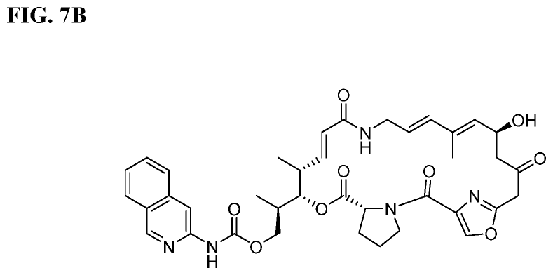

Streptogramin antibiotics (also known as virginiamycins, madumycins, pristinamycins) are produced by several species of Streptomyces and comprise two structurally distinct subgroups (FIG. 1). Group A streptogramins are characterized by a 23-membered macrocycle derived biosynthetically from polyketide synthase-non-ribosomal peptide synthase megaenzymes. Group B streptogramins are 19-membered cyclic depsipeptides assembled by non-ribosomal peptide synthases. Streptogramins have been used as feed additives for livestock for decades,¹ but were not approved by the FDA until the introduction of quinupristin-dalfopristin (Synercid®, a combination of 7 and 2) in 1999. The therapeutic use of this combination therapy is limited by its IV-only formulation and its narrow spectrum of activity, and it is reserved for hospitalized patients with multi drug-resistant skin and skin-structure infections as well as for patients with bacteremia caused by vancomycin-resistant Enterococcus faecium.² An orally bioavailable combination of semisynthetic streptogramins known as NXL-103 (flopristin-linopristin, not depicted) underwent phase-II clinical trials in 2011, but has not progressed further in the clinic.³

The two components of quinupristin-dalfopristin are derived from virginiamycin SI (6) and virginiamycin M1 (1) which are in turn obtained from the fermentation broth of Streptomyces pristinaespiralis,^{2,4} This semisynthetic approach has enabled commercial-scale production of this combination therapy, but it does not permit broad exploration of structure-activity relationships of each component. New methods to modify streptogramins, informed by recent crystallographic data of several streptogramins bound to the bacterial ribosome,⁵ would enable further optimization to overcome the limitations of this class. Dalfopristin and quinupristin bind to the 70S E. Coli ribosome (PDB 2W26). Group A streptogramins bind to the peptidyltransferase center and directly interfere with the catalytic activity of the ribosome. Methylation of A2503 and A2053 interrupt group A and group B binding, respectively. Group A streptogramins are susceptible to drug deactivation by acetyltransferases, with combination therapy being beneficial for certain organisms.

Chemical syntheses of group A streptogramins, the major components of combination therapies in the class, have appeared in the literature. Three routes to virginiamycin M2 (3) have been reported⁶ including an elegant synthesis by Wu and Panek that represents the shortest and most efficient fully synthetic route to a streptogramin reported to-date (6% overall yield over 10 linear steps from an allylsilane precursor).⁶⁶ Syntheses of madumycin II (5)⁷ and of closely related streptogramins⁸ have also been disclosed. To the best of our knowledge, fully synthetic routes madumycin I (4) and virginiamycin M1 (1) have not been developed. We were particularly interested in access to 1, as its dehydroproline function serves as a handle for the installation of sidechains (e.g., l->2) that increase water solubility and are permitted in its binding pocket.^{56,56} Herein we describe a concise, modular synthesis of group A streptogramins.

Scheme 1. Synthesis of madumycins I (4) and II (5) by the convergent assembly of 7 simple building blocks. “Reagents and conditions: (a) 10 (0.5 equiv), TfOH (0.45 equiv), DCM, −78° C., 1 h, then a solution of isopropanol (1.1 equiv), 8 (1 equiv), 9 (1.25 equiv) in DCM (slow addition over 2 h), 1.5 h, 94%, 87% ee; (b) propargylamine (4 equiv), AlMe₃ (4 equiv), 0° C. to 23° C., then 11, DCM, 40° C., 3 h, 90%; (c) CuCN (2 equiv), n-BuLi (4.2 equiv), Bu₃SnH (4.2 equiv), 1 h, −78° C., 100%; (d) 12 (1 equiv), 13 (1.5 equiv), DCC (1.6 equiv), DMAP (0.2 equiv), 6 h, then Et₂NH (480 equiv), DCM, 23° C., 3 h, 88%; (e) 16 (1.1 equiv), TiCl₄ (1.2 equiv), ⁱPr₂EtN (1.2 equiv), 2 h, 15 (1 equiv, slow addition over 30 min), 10 min, 64%; (f) 17 (1 equiv), 2,6-lutidine (2 equiv), TBSOTf (1.2 equiv), DCM, 0° C., 30 min, 92%; (g) 19 (2 equiv), n-BuLi (4 equiv), 30 min, then 18 (1 equiv, slow addition over 30 min), THF, −78° C., 30 min, 84%; (h) 14 (1 equiv), 20 (1.1 equiv), ⁱPr₂EtN (2 equiv), HATU (1.5 equiv), DCM, 23° C., 5 h, 88%; (i) 21 (1 equiv), JackiePhos (0.2 equiv), Pd₂dba₃ (0.1 equiv), toluene, 50° C., 3 h, 62%; (j) Bu₄NF (10 equiv), Im.HCl (10 equiv), THF, 23° C., 12 h, 91%; (k) 4 (1 equiv), Et₂BOMe (1.2 equiv), 15 min, then NaBH₄ (2 equiv), THF:MeOH 4:1 (v/v), −78° C., 3 h, 86%. TBS=tert-butyldimethylsilyl, TfO=trifluoromethanesulfonate, DCC=dicyclohexylcarbodiimide, DMAP=4-dimethylaminopyridine, Fmoc=9-fluorenylmethoxycarbonyl Im=imidazole, DCM=dichloromethane, THF=tetrahydrofuran.

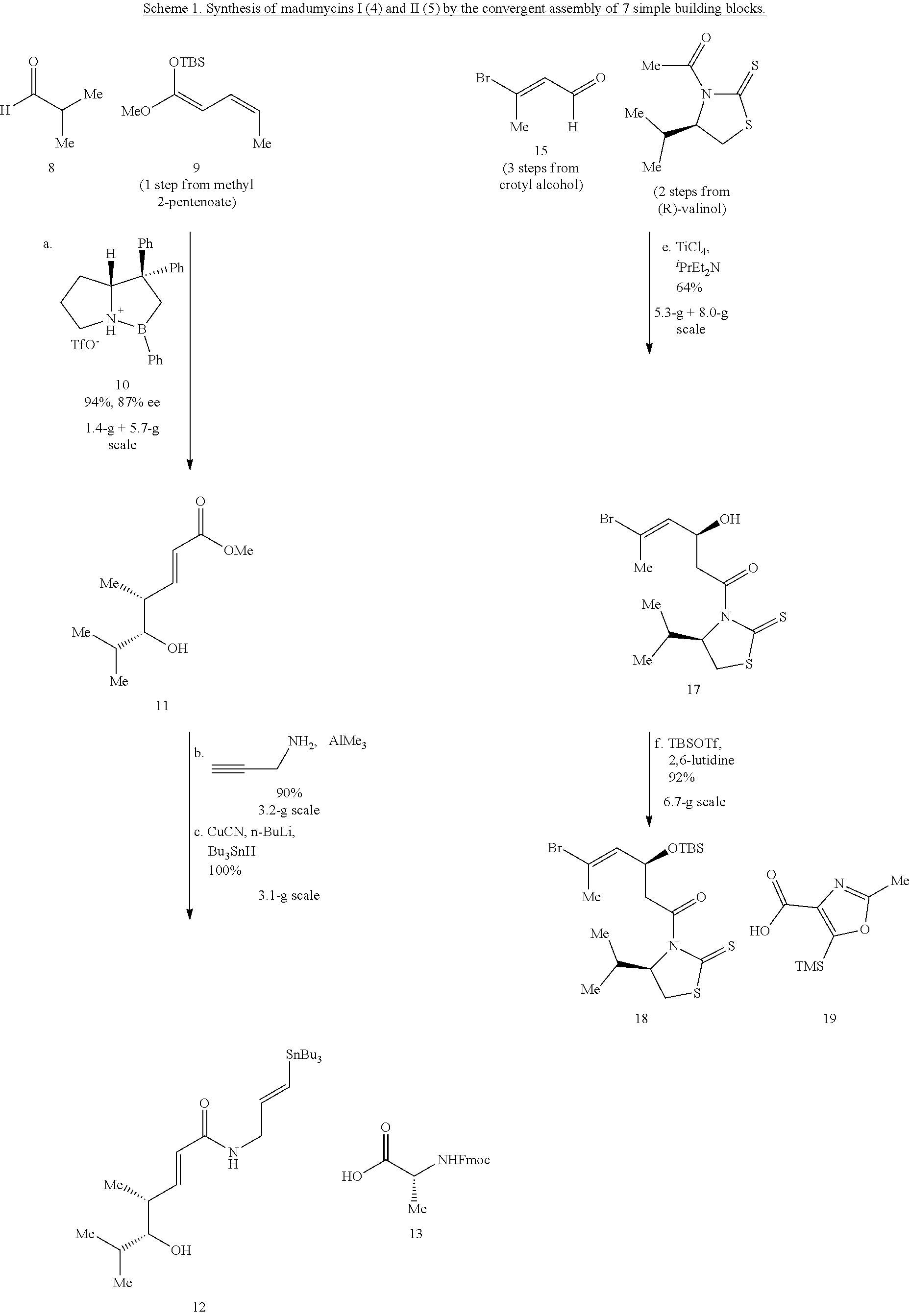

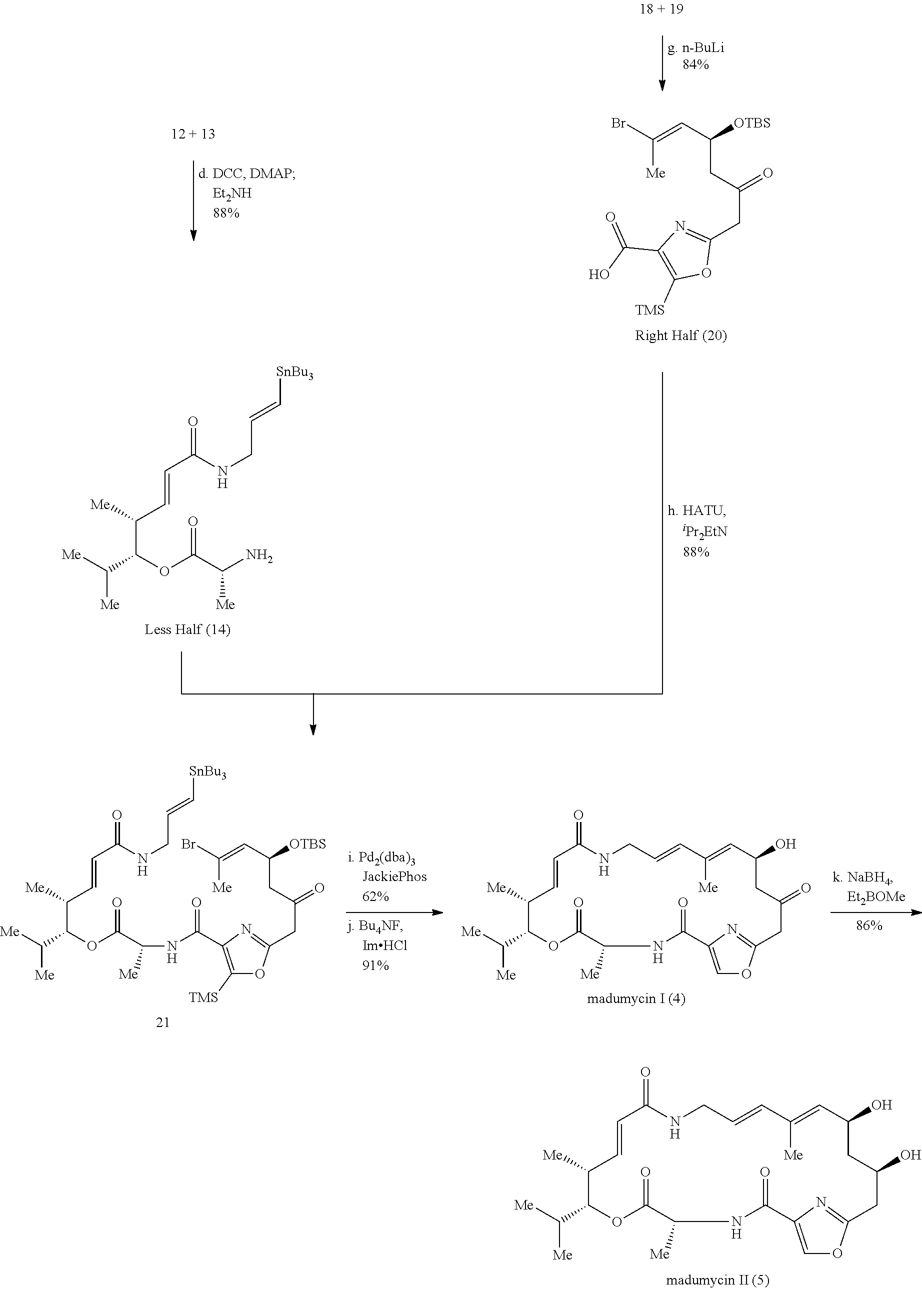

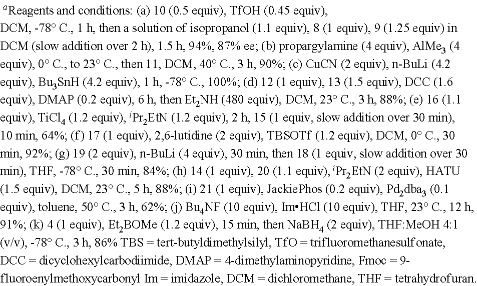

Our approach to madumycins I (4) and II (5) from seven simple building blocks is depicted in Scheme 1 (building blocks in bold) and within the Figures. Our synthesis of the left half commences with a Mukaiyama-type vinilogous aldol reaction between silyl dienol ether 8 (available in one step from methyl 2-pentenoate) and isobutyraldehyde (7) catalyzed by 9 to provide enoate 10 in 94% yield and 87% ee.⁹ This step serves to set two of the stereocenters found in the final product and is readily performed on multigram-scale.¹⁰ Amidation of the methyl ester in 11 with propargylamine in the presence of trimethylaluminum followed by hydrostannylation of the resulting terminal alkyne produces 12 in 90% overall yield. Notably, these two steps are carried out in the presence of an unprotected secondary alcohol, obviating the need for additional protection/deprotection steps. D-alanine was introduced as its Fmoc-carbamate (13) using DCC in the presence of catalytic DMAP followed by the addition of diethylamine to cleave the Fmoc group in a single operation in 88% yield. The synthesis of left half 14 proceeds in 4 steps in 74% overall yield from building blocks 8 and 9 (5 steps from commercially available materials), and has been conducted on multigram-scale. It is important to note that three of the steps in the sequence incorporate building blocks that are amenable to structural diversification, as we will demonstrate (vide infra).

Our synthesis of right half (20) commences with the aldol coupling of (E)-3-bromobut-2-enal (15, available in 3 steps from crotyl alcohol) and acetyl thiazolidinethione 16, providing 17 in 64% yield as a single diastereomer. We found that the ß-hydroxycarbonyl in 17 was too labile for the subsequent steps in the synthesis, and thus the secondary alcohol is shielded as its tert-butyldimethylsilyl ether in 92% yield to provide ß-silyloxyimide 18. Treatment with the dianion of oxazole 19, which contains a trimethylsilyl function at C₅ to prevent aryl deprotonation,¹¹ directly provides right half acid 20 in 84% yield. This enabling step allows the carboxylic acid to be directly introduced, obviating the need for a subsequent deprotection step. The route to the right half proceeds in 49% overall yield from 15 and 16 and has enabled the preparation of over 10 grams of 20.

The coupling of left half 14 to right half 20 is accomplished using HATU in the presence of diisopropylethylamine to provide macrocycle precursor 21 in 88% yield. We next explored macrocyclization by means of an intramolecular Stille cross coupling reaction. Macrocyclization reactions are often challenging steps in fully synthetic approaches to streptogramins;^{6-7,8b,8c12} indeed, Pattenden and coworkers found a Stille macrocyzliation to be a limiting step (30% yield with Pd₂dba₃ and Ph₃As) in their synthesis of 14,15-anhydrovirginiamycin M2.^{8a,8b} Optimization of the Stille macrocyclization reaction on a related substrate (see Example 2) revealed that sterically hindered phosphine ligands provided the highest isolated yields. Buchwald's JackiePhos, a ligand designed to facilitate challenging transmetallations, was found to be optimal.¹³

TABLE 1
Optimization of the Stille macrocyclization.

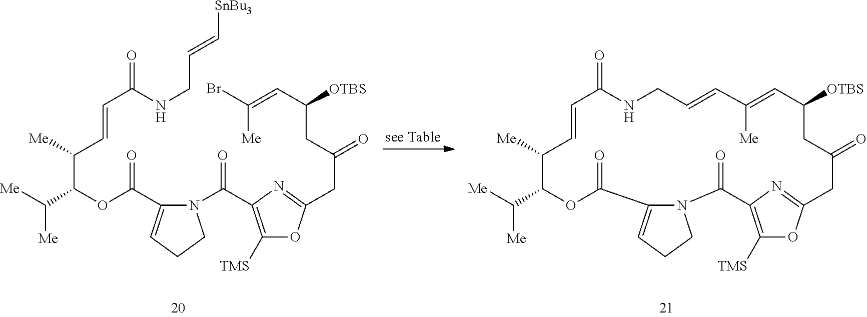

| | | | solvent | | |
|---|---|---|---|---|---|
| entry | ligand (mol %) | Pd source (mol %) | (conc. ##) | conditions | yield (%) |
| 1 | AsPh₃ (80) | Pd₂(dba)₃ (10 × 2) | THF (2 mM) | 80° C., 72 h | nd |
| 2 | LiCl (500) | Pd₂(dba)₃ (10) | DMF (2 mM) | 50° C., 40 h | nd |
| 3 | — | Pd(PPh₃)₄ (20) | toluene (2 mM) | 50° C., 40 h | 15 |
| 4 | RuPhos (30) | Pd₂(dba)₃ (15) | toluene (2 mM) | 80° C., 24 h | 31 |
| 5 | BrettPhos (30) | Pd₂(dba)₃ (15) | toluene (2 mM) | 80° C., 24 h | 39 |
| 6 | JackiePhos (30) | Pd₂(dba)₃ (15) | toluene (2 mM) | 80° C., 24 h | 46 |
| 7 | JackiePhos (30) | Pd₂(dba)₃ (15) | toluene (2 mM) | 50° C., 72 h | 34 |
| 8 | JackiePhos (30) | Pd₂(dba)₃ (15) | toluene (2 mM) | 110° C., 24 h | 30 |
| 9 | JackiePhos (30) | Pd₂(dba)₃ (15) | toluene (10 mM) | ##° C., ##h | tbd |

| | | | | |
|---|---|---|---|---|
| 10 | JackiePhos-Pd-G3 (30) | toluene (2 mM) | 80° C., 72 h | 47 |

Thus, intramolecular Stille macrocyclization of 21 proceeds at 50° C. in 4 h with 20% catalyst loading to provide protected macrocycle in 62% yield. Highly dilutive conditions, which frequently plague macrocyclization reactions, were not required: this reaction proceeds without appreciable amounts of dimeric or polymeric byproducts even at 0.01 M. Removal of the silyl groups is accomplished with buffered tetrabutylammonium fluoride to provide madumycin I (4) with an overall yield of 37% from 8 and 9 or 25% from and 16. Treatment of 4 with sodium borohydride in the presence of diethylmethoxyborane¹⁴ provides madumycin II (5) in 86% yield as a single diastereomer, and represents the first reported interconversion of these natural products.

Scheme 2: Syntheses of virginiamycins M1 (1) and M2 (3). “Reagents and conditions: (a) 22 (1.5 equiv), 12 (1 equiv), DCC (1.6 equiv), DMAP (0.2 equiv), 6 h, then Et₂NH (480 equiv), DCM, 23° C., 3 h, 88%; (b) 23 (1 equiv), 20 (1.1 equiv), ⁱPr₂EtN (2 equiv), HATU (1.5 equiv), DCM, 23° C., 5 h, 87%; (c) JackiePhos (0.2 equiv), Pd₂dba₃ (0.1 equiv), toluene, 50° C., 3 h, 59%; (d) 24 (1 equiv), Bu₄NF (10 equiv), Im.HCl (10 equiv), THF, 23° C., 12 h, 82%; (e) PhIO (1.1 equiv), DCM, 23° C., 30 min, 92%; (f) 20 (1.25 equiv), Ghosez reagent (1.3 equiv), 2,6-lutidine (2.5 equiv), 2 h, then 25 (1 equiv), DCM, 23° C., 12 h, 65%; (g) JackiePhos (0.3 equiv), Pd₂dba₃ (0.15 equiv), toluene, 80° C., 24 h, 47%; (h) 26 (1 equiv), Bu₄NF (10 equiv), Im.HCl (10 equiv), THF, 23° C., 12 h, 85%. TfO=trifluoromethanesulfonate, Fmoc=9-fluorenylmethoxycarbonyl, HATU=1-[Bis(dimethylamino)methylene]-1H-1,2,3-triazolo[4,5-b]pyridinium-3-oxidhexafluorophosphate, TBS=tert-butyldimethylsilyl TMS=trimethylsilyl, dba=dibenzylideneacetone, Im=imidazole, DCM=dichloromethane, THF=tetrahydrofuran.

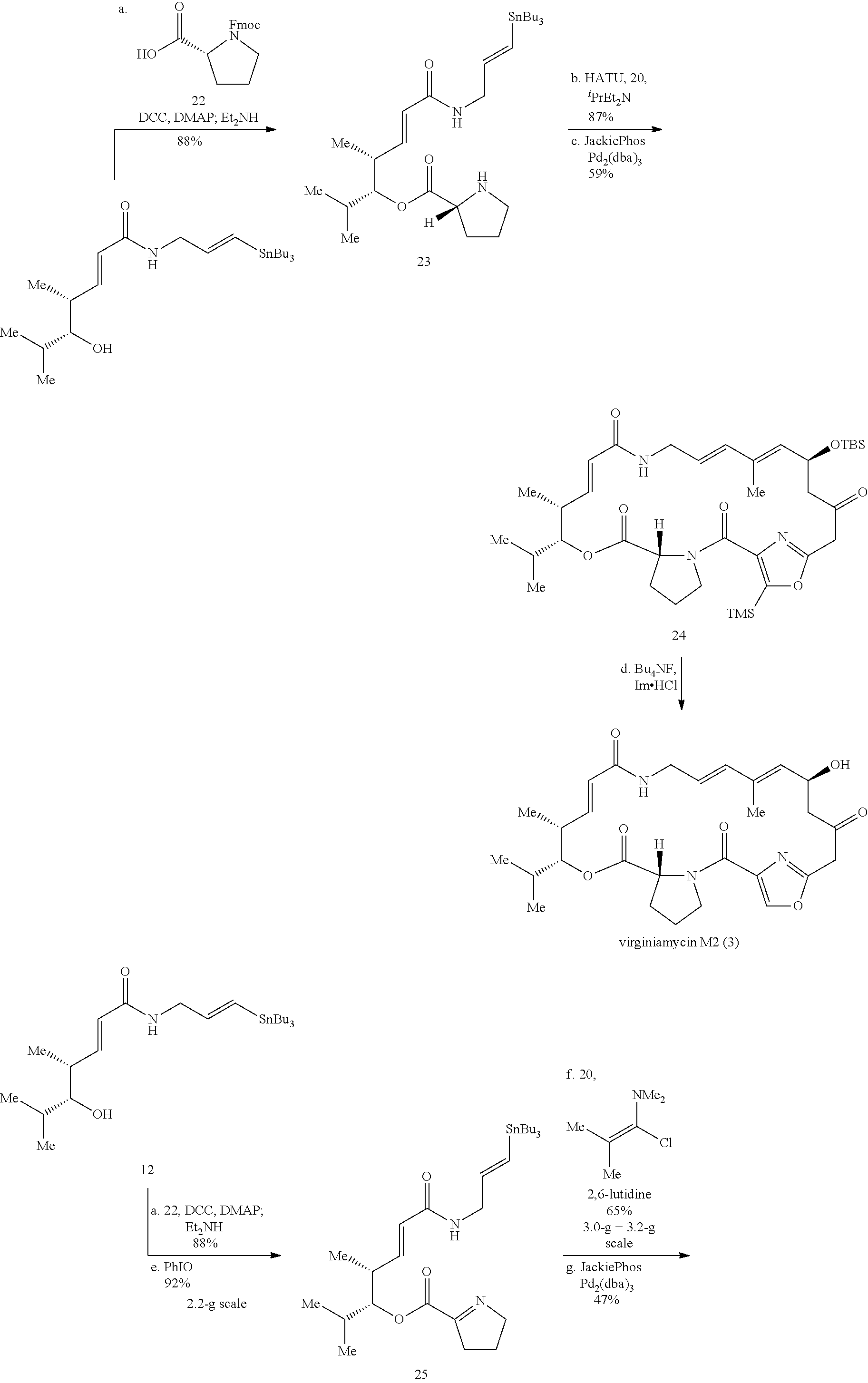

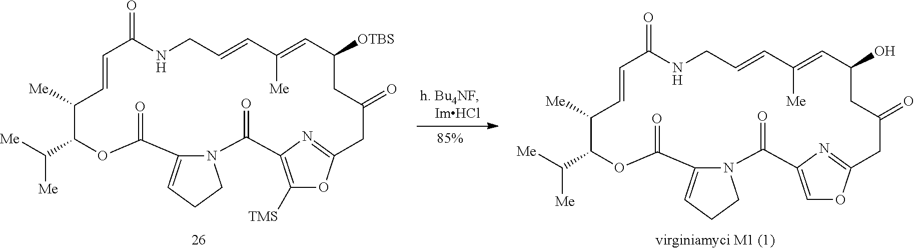 [SMILES: CC1=C\[C@@H](O[Si](C)(C)C(C)(C)C)CC(=O)Cc2nc(c([Si](C)(C)C)o2)C(=O)N2CCC=C2C(=O)O[C@H](C(C)C)[C@H](C)/C=C/C(=O)NC\C=C\1 ; CC1=C\[C@@H](O)CC(=O)Cc2nc(co2)C(=O)N2CCC=C2C(=O)O[C@H](C(C)C)[C@H](C)/C=C/C(=O)NC\C=C\1]

To demonstrate the modularity of our route, we next applied it to the syntheses of virginiamycins M1 (1) and M2 (2) as depicted in Scheme 2. DCC/DMAP-mediated esterification of alcohol 12 with Fmoc-protected D-proline (22) followed by the addition of Et₂NH provides proline ester 23 in 88% yield. Coupling with the right half (20), macrocyclization, and desilylation provides virginiamycin M2 (3) in yields comparable to those in Scheme 1. Access to virginiamycin M1 (1) proved to be more challenging due to the 2,3-dehydroproline function. Coupling of 12 and 22 proceeds as above, and the resulting secondary amine 23 is oxidized efficiently and selectively to imine 25 in the presence of iodosylbenzene. We found that several amide bond-forming reagents were ineffective at coupling 25 to right half 20, potentially due to the comparatively lower nucleophilicity of the 1,2-dehydroproline function relative to other amines. Successful coupling is achieved by initial conversion of 20 to an acid chloride with Ghosez's reagent¹⁵ followed by treatment with imine 25, which reliably provides macrocycle precursor in 65% yield on multigram scale. Macrocyclization requires increased catalyst loading (30%) and slightly increased temperature (80° C.) and provides 26 in 47% yield. Desilylation efficiently provides virginiamycin M1 (1).

The left (14, 23, 25) and right (20) halves are obtained in linear sequences of 3-5 steps from the simple chemical building blocks (represented in bold in Schemes 1 and 2), or <6 steps from commercially available materials. After coupling of the halves, only 2-3 steps are required to reach the final products. The short linear sequences are a direct consequence of the high degree of convergency of the route. Additionally, every non-hydrogen atom in the final products (e.g., methyl) arises from those found in the seven simple building blocks. Finally, each step has proven to be robust and scalable, which will facilitate access sufficient quantities of candidates for both microbiological testing and animal studies. We are currently applying our approach to the syntheses of several non-natural analogs of group A streptogramins with the aims of improving their pharmacological properties, expanding their spectrum of activity, and increasing their potency against multidrug-resistant strains of pathogenic bacteria.

### Example 2: Synthesis and Characterization

General Experimental Procedures:

All reactions were performed in flame-dried glassware fitted with rubber septa under a positive pressure of nitrogen or argon, unless otherwise noted. Air- and moisture-sensitive liquids were transferred via syringe or stainless steel cannula. Solutions were concentrated by rotary evaporation below 35° C. Analytical thin-layer chromatography (TLC) was performed using glass plates pre-coated with silica gel (0.25-mm, 60-Å pore size, 230-400 mesh, SILICYCLE INC) impregnated with a fluorescent indicator (254 nm). TLC plates were visualized by exposure to ultraviolet light (UV), and then were stained by submersion in a basic aqueous solution of potassium permanganate or with an acidic ethanolic solution of anisaldehyde, followed by brief heating on a hot plate.

Materials:

Commercial solvents and reagents were used as received, unless otherwise noted.

Instrumentation: Proton nuclear magnetic resonance (¹H NMR) spectra and carbon nuclear magnetic resonance (¹³C NMR) spectra were recorded on 400 MHz Bruker Avance III HD 2-channel instrument NMR spectrometers at 23° C. or 50° C. Proton chemical shifts are expressed in parts per million (ppm, 5 scale) and are referenced to residual protium in the NMR solvent (CHC1₃: δ 7.26). Carbon chemical shifts are expressed in parts per million (ppm, 5 scale) and are referenced to the carbon resonance of the NMR solvent (CDC1₃: δ 77.0). Data are represented as follows: chemical shift, multiplicity (s=singlet, d=doublet, t=triplet, q=quartet, dd=doublet of doublets, dt=doublet of triplets, sxt=sextet, m=multiplet, br=broad, app=apparent), integration, and coupling constant (J) in hertz (Hz). Optical rotations were measured using a JASCO P-2000 polarimeter. High-resolution mass spectra were obtained at the QB3/Chemistry Mass Spectrometry Facility at University of California, Berkeley using a Thermo LTQ-FT mass spectrometer.

Scheme I-1 Synthesis of left half (14):

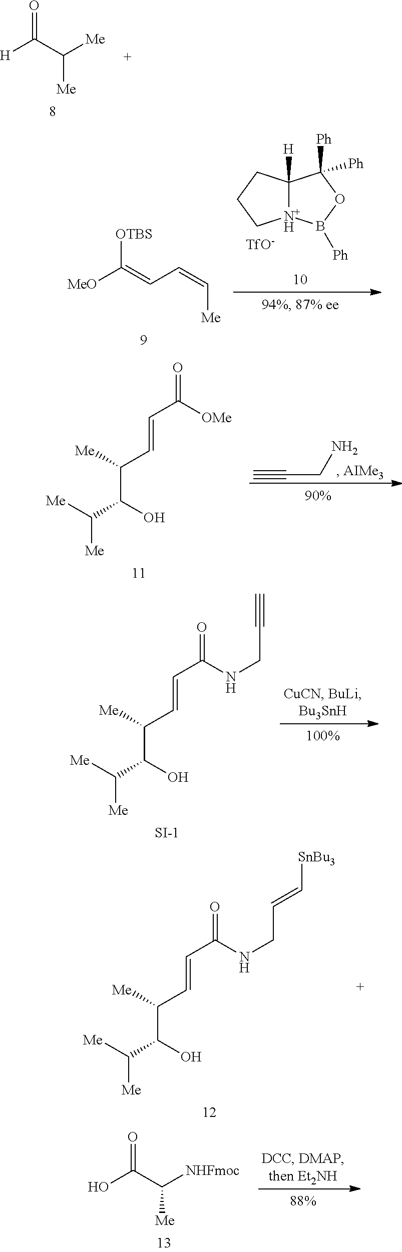

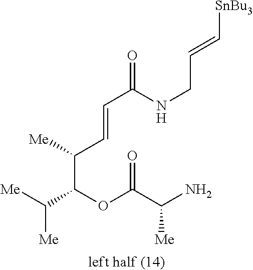

Mukaiyama Aldol Product 11:

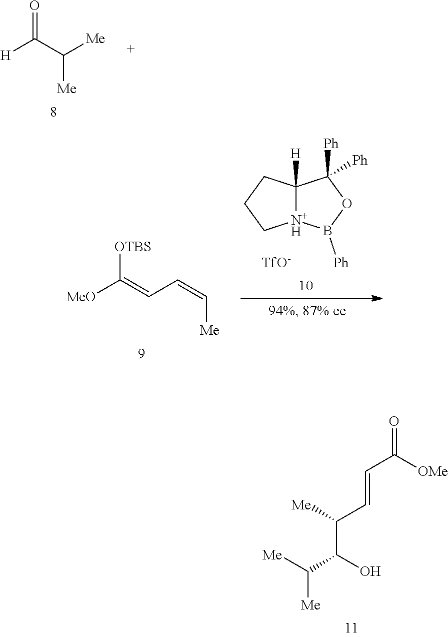

Phenylboronic acid (1.217 g, 9.985 mmol) and (S)-diphenyl(pyrrolidin-2-yl)methanol (2.529 g, 9.985 mmol) in toluene (50 mL) was heated at reflux for 12 hours under N₂, The reaction mixture was concentrated in vacuum, dried over pump and 80 mL CH₂Cl₂ was added. The solution was cooled to −78° C. and trifluoromethanesulfonic acid (1.348 g, 0.795 mL, 8.986 mmol) was added at this condition at −78° C. After TfOH was totally dissolved, a mixture of (8) (1.44 g, 1.82 mL, 20.0 mmol), (9) (5.70 g, 25.0 mmol), and propan-2-ol (1.32 g, 1.68 mL, 22.0 mmol) in CH₂Cl₂ (20 mL) was added dropwise over 2 hours. The mixture was stirred at −78° C. for another 1.5 h and quenched with sat. aq. NaHCO₃. The mixture was extracted with DCM, dried over Na₂SO₄, filtered and concentrated under reduced pressure. The crude residue was purified by flash chromatography (silica gel, Hexane:EtOAc=10:1 to 6:1) to afford methyl Mukaiyama Aldol product 11 (3.48 g, 94%) as a colorless oil.

TLC (ethyl acetate:hexane=1:6): R_{f}=0.25 (UV, KMnO₄); [α]²³_{D}=+23.5 (c=1.0, CHCl₃); ¹H NMR (400 MHz, CDCl3) δ 6.92 (dd, J=15.7, 8.1 Hz, 1H), 5.86 (dd, J=15.7, 1.2 Hz, 1H), 3.72 (s, 3H), 3.26 (t, J=5.8 Hz, 1H), 2.59-2.39 (m, 1H), 1.78-1.64 (m, 1H), 1.59 (br s, 1H), 1.09 (d, J=6.7 Hz, 3H), 0.92 (d, J=6.8 Hz, 3H), 0.90 (d, J=6.8 Hz, 3H; ¹³C NMR (100 MHz, CDCl₃) δ 167.1, 152.2, 120.4, 80.0, 51.4, 39.9, 30.9, 19.6, 16.5, 13.9; HRMS-EI m/z calcd for C10H19O3 [M+H]⁺ 187.1329, found 187.1331.

Amide SI-1:

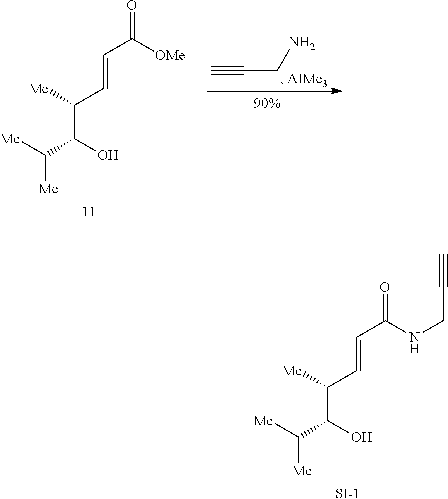

To a stirred solution of propargylamine (3.79 g, 4.40 mL, 68.7 mmol) in dry CH₂Cl₂ (115 mL, 0.6 M) was added 1 M AlMe₃ in heptane (4.95 g, 68.7 mL, 68.7 mmol) dropwise at 0° C. under N₂ atmosphere over 30 min. The mixture was stirred at rt for 30 min, and (1)(3.2 g, 17.2 mmol) in CH₂Cl₂ (20 mL) was added in one portion. The mixture was heated under reflux for 3 h, cooled to 0° C., quenched with MeOH carefully and saturated aqueous solution of Rochelle salt was added. After stirring for 1 hour and the aqueous layer was abstracted with DCM. The combined the organic layer was washed with water, dried by Na₂SO₄, and concentrated under vacuum. The residue was purified by flash chromatography (silica gel, EtOAc:hexane=1:1) to afford (4R,5R,E)-5-hydroxy-4,6-dimethyl-N-(prop-2-yn-1-yl)hept-2-enamide (SI-1) (3.22 g, 90%) as a white solid.

TLC (ethyl acetate:hexane=1:1): R_{f}=0.15 (UV); [α]²⁴_{D}=+29.7 (c=1.0, CH₂Cl₂); ¹H NMR (400 MHz, CDCl₃) δ 6.84 (dd, J=15.4, 7.9 Hz, 1H), 5.82 (dd, J=15.4, 1.2 Hz, 1H), 5.78 (s, 1H), 4.12 (dd, J=5.3, 2.6 Hz, 2H), 3.30-3.22 (m, 1H), 2.56-2.43 (m, 1H), 2.24 (t, J=2.6 Hz, 1H), 1.80-1.65 (m, 1H), 1.59 (d, J=5.1 Hz, 1H), 1.08 (d, J=6.7 Hz, 3H), 0.92 (d, J=6.7 Hz, 6H); ¹³C NMR (100 MHz, CDCl₃) δ 165.4, 148.4, 122.6, 79.4, 79.2, 71.7, 39.6, 30.8, 29.2, 19.7, 16.7, 13.9; HRMS-ESI m/z calcd for C₁₂H₂₀NO₂ [M+H]⁺ 210.1489, found 210.1487.

Vinyl Stannane 12:

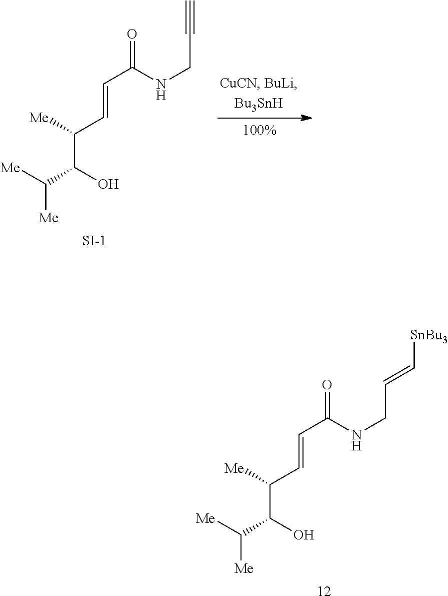

To a suspension of copper cyanide (2.65 g, 29.6 mmol) in dry THF (200 mL) was added 2.5 M n-butyllithium in hexane (24.9 mL, 62.2 mmol) dropwise at −78° C. under N₂. The mixture was stirred for 30 min and became a clear solution. To this solution was added tributyl stannane (18.1 g, 16.8 mL, 62.2 mmol) dropwise at −78° C. After stirring for 30 min, a solution of (SI-1) (3.1 g, 14.8 mmol) in THF (15 mL) was added dropwise. 1 hour later, the reaction was quenched with aqueous NH₄Cl, stirred for 1 hour and extracted with EtOAc. The resulting organic layer was washed with aqueous ammonium hydroxide, water and brine, dried over Na₂SO₄ and concentrated under vacuum. The residue was purified by flash chromatography (silica gel, EtOAc:Hexane=0:1 to 1:3) to afford (4R,5R,E)-5-hydroxy-4,6-dimethyl-N-((E)-3-(tributylstannyl)allyl)hept-2-enamide (12) (7.4 g, 100%) as a colorless oil.

TLC (ethyl acetate:hexane=1:3): R_{f}=0.25 (UV); [α]²⁴_{D}=+10.6 (c=1.0, CHCl₃); NMR (400 MHz, CDCl₃) δ 6.82 (dd, J=15.4, 7.9 Hz, 1H), 6.12 (dt, J=19.0, 1.5 Hz, 1H), 5.97 (dt, J=19.0, 5.1 Hz, 1H), 5.83 (dd, J=15.4, 1.2 Hz, 1H), 5.58 (hr s, 1H), 4.04-3.94 (m, 2H), 3.26 (q, J=5.6 Hz, 1H), 2.56-2.42 (m, 1H), 1.80-1.67 (m, 1H), 1.53-1.40 (m, 6H), 1.29 (m, 6H), 1.09 (d, J=6.6 Hz, 3H), 0.94-0.83 (m, 21H); ¹³C NMR (100 MHz, CDCl₃) δ 165.5, 147.4, 143.4, 130.4, 123.3, 79.2, 44.9, 39.6, 30.8, 29.0, 27.2, 19.7, 16.7, 14.0, 13.7, 9.4; HRMS (CI/NH₃) m/z calcd for C₂₄H₄₇NNaO₂Sn [M+Na]⁺ 524.2521, found 524.2515.

Amine 14:

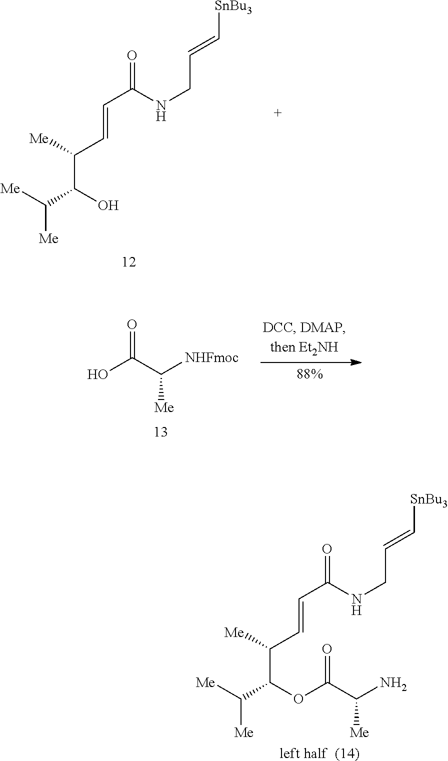

DCC (1.72 g, 8.31 mmol) was added to a stirred solution of (13) (2.43 g, 7.79 mmol), (12) (2.6 g, 5.2 mmol) and DMAP (0.127 g, 1.04 mmol) in dry CH₂Cl₂ (52 mL) in one portion at RT and the reaction mixture was stirred overnight. Diethyl amine (26 mL) was added to this mixture. After additional 3 hours, the mixture was filtered over a pad of celite and the filter cake was washed with DCM. The solvent was removed under vacuum and the residue was purified by flash chromatography (silica gel, NH₄OH:MeOH:DCM=0.2:1:100 to 0.2:1:50) to afford amine 14 (2.86 g, 94%) as colorless slurry.

TLC (MeOH:DCM=1:25): R_{f}=0.25 (UV, KMnO₄); [α]²⁴_{D}=+4.6 (c=0.5, CHCl₃); NMR (400 MHz, CDCl₃) δ 6.70 (dd, J=15.4, 8.0 Hz, 1H), 6.11 (dt, J=19.0, 1.4 Hz, 1H), 5.96 (dt, J=19.1, 5.1 Hz, 1H), 5.82 (dd, J=15.4, 1.1 Hz, 1H), 5.60 (t, J=5.8 Hz, 1H), 4.80 (dd, J=7.1, 5.1 Hz, 1H), 4.04-3.88 (m, 2H), 3.55 (q, J=7.0 Hz, 1H), 2.71-2.55 (m, 1H), 1.97-1.82 (m, 1H), 1.61 (br s, 2H), 1.52-1.41 (m, 6H), 1.34 (q, J=6.9 Hz, 1H), 1.36-1.23 (m, 6H), 1.02 (d, J=6.7 Hz, 3H), 0.93-0.80 (m, 21H); ¹³C NMR (100 MHz, CDCl₃) δ 176.4, 165.2, 145.1, 143.3, 130.4, 123.9, 80.2, 50.2, 44.9, 38.2, 29.8, 29.0, 27.2, 20.9, 19.6, 16.6, 14.9, 13.7, 9.4; HRMS (CI/NH3) m/z calcd for C₂₇H53N₂O₃Sn [M+H]+ 573.3073, found 573.3062.

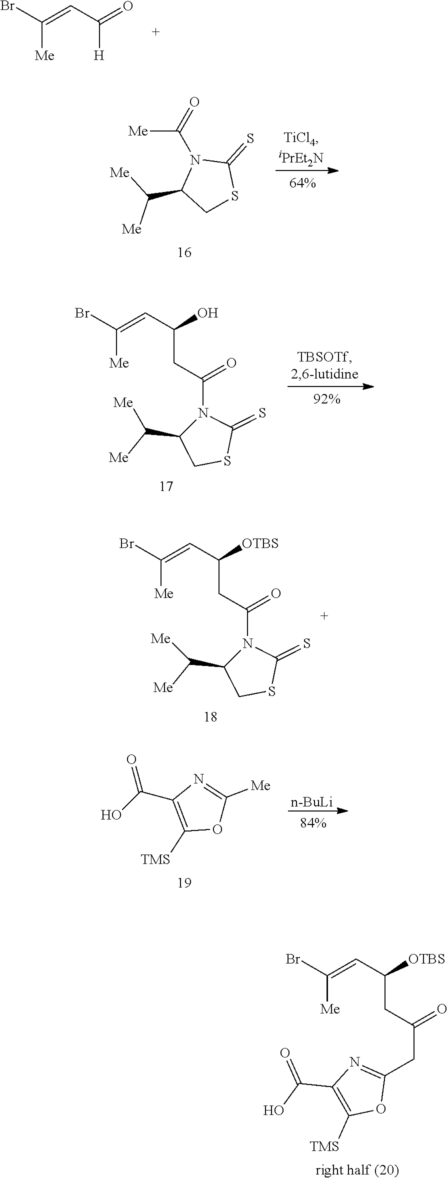

ß-Hydroxyl Amide 17:

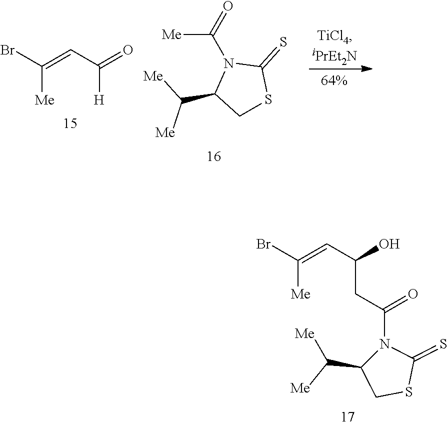

To a solution of (16) (7.96 g, 39.1 mmol) in DCM (80 mL, 0.25 M) was added 1M TiCl₄ in DCM (42.7 mL, 42.7 mmol) dropwise at −78° C. under N₂ atmosphere. 5 min later, DIPEA (5.52 g, 7.46 mL, 42.7 mmol) was added via syringe pump over 30 min and the reaction mixture was stirred for 2 hours at −78° C. A solution of 15 (5.3 g, 35.6 mmol) in DCM (10 mL) was added to this mixture via syringe pump over 30 min, the reaction was kept on stirring for 30 min at −78° C. and quenched with water. After warming to RT, the aqueous layer was abstracted with EtOAc, the combined organic layer was washed with water and brine, dried over Na₂SO₄ and concentrated under vacuum. The residue was purified by flash chromatography (silica gel, EtOAc:Hexane=1:10 to 1:6 to 1:2.5) to afford ß-hydroxyl amide 17 (8.3 g, 64%) as a yellow oil.

TLC (ethyl acetate:hexane=1:5): R_{f}=0.25 (UV); [α]²⁴_{D}=−320 (c=1.0, CHCl₃); ¹H NMR (400 MHz, CDCl₃) δ 5.95 (dq, J=8.9, 1.3 Hz, 1H), 5.14 (ddd, J=7.7, 6.3, 1.1 Hz, 1H), 4.80 (tdd, J=8.4, 4.4, 3.3 Hz, 1H), 3.59 (dd, J=17.6, 3.3 Hz, 1H), 3.53 (dd, J=11.5, 8.0 Hz, 1H), 3.32 (dd, J=17.7, 8.4 Hz, 1H), 3.03 (dd, J=11.5, 1.1 Hz, 1H), 2.98 (d, J=4.6 Hz, 1H), 2.45-2.25 (m, 1H), 2.32 (d, J=1.4 Hz, 3H), 1.05 (d, J=6.8 Hz, 3H), 0.97 (d, J=6.9 Hz, 3H); ¹³C NMR (100 MHz, CDCl₃) δ 202.9, 171.8, 132.4, 124.2, 71.3, 65.7, 44.8, 30.7, 30.6, 24.1, 19.0, 17.7; HRMS-ESI m/z calcd for C₁₂H₁₇BrNO₂S₂ [M−H]⁻ 349.9890, found 349.9886.

TBS Ether 18:

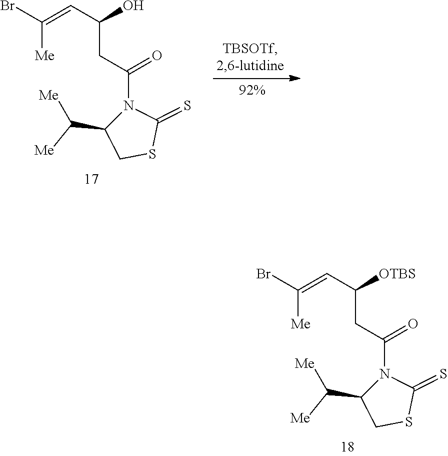 [SMILES: C/C(Br)=C\[C@@H](O)CC(=O)N1C(=S)SC[C@H]1C(C)C ; C/C(Br)=C\[C@H](CC(=O)N1C(=S)SC[C@H]1C(C)C)O[Si](C)(C)C(C)(C)C]

To a solution of (17) (4.71 g, 13.4 mmol) and 2,6-lutidine (2.87 g, 3.1 mL, 26.78 mmol) in DCM (134 mL) was added TBSOTf (4.24 g, 3.69 mL, 16.0 mmol) drop wise at 0° C. After stirring for 30 min, the mixture was washed with water and brine, dried over Na₂SO₄ and concentrated under vacuum. The resulting residue was purified by flash chromatography (silica gel, EtOAc:Hexane=1:20) to afford TBS ether 18 (5.76 g, 92%) as a light yellow oil.

TLC (ethyl acetate:hexane=1:50): R_{f}=0.20 (UV); [α]²⁴_{D}=−479 (c=1.0, CHCl₃); NMR (400 MHz, CDCl₃) δ 5.87 (dq, J=8.9, 1.3 Hz, 1H), 5.03 (ddd, J=7.6, 6.2, 1.1 Hz, 1H), 4.96-4.86 (m, 1H), 3.63 (dd, J=16.5, 8.3 Hz, 1H), 3.47 (dd, J=11.5, 7.9 Hz, 1H), 3.18 (dd, J=16.5, 4.3 Hz, 1H), 3.03 (dd, J=11.4, 1.1 Hz, 1H), 2.36 (dq, J=13.5, 6.8 Hz, 1H), 2.31 (d, J=1.3 Hz, 3H), 1.06 (d, J=6.8 Hz, 3H), 0.97 (d, J=7.0 Hz, 3H), 0.84 (s, 9H), 0.05 (s, 3H), 0.05 (s, 3H); ¹³C NMR (100 MHz, CDCl3) δ 202.8, 170.7, 134.5, 121.7, 71.7, 67.2, 45.6, 30.9, 30.8, 25.7, 24.1, 19.1, 18.0, 17.8, −4.5, −5.0; HRMS-EI m/z calcd for C₁₈H₃₂BrNO₂S₂Si [M−H]⁻ 465.0833, found 465.0819.

Carboxylic Acid 20:

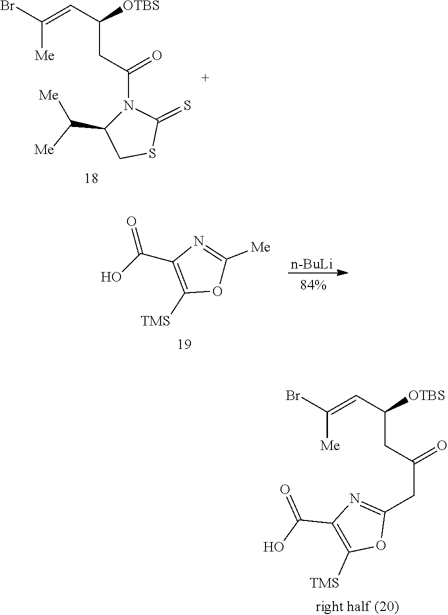

To a solution of 19 (3.42 g, 17.1 mmol) in dry THF (170 mL) was added 2.5 M n-Butyllithium (13.717 mL, 34.3 mmol) in hexane dropwise at −78° C. under N₂ atmosphere and the mixture was stirred under this condition for 30 min. A solution of (18) (4 g, 8.57 mmol) in THF (25 mL) was added to this solution over 30 min at −78° C. After stirring for 30 min, the reaction was quenched with water and 1 M KHSO₄ (50 mL) and abstracted with EtOAc twice. The combined organic layer was washed with water and brine, dried over Na₂SO₄ and concentrated under vacuum. The residue was purified by flash chromatography (silica gel, EtOAc:Hexane=1:7.5 to 1:3) to afford carboxylic acid 20 (3.64 g, 84%) as a yellow foam.

TLC (MeOH:DCM=1:20): R_{f}=0.30 (UV); [α]²⁴_{D}=−24.5 (c=1.0, CHCl₃); ¹H NMR (400 MHz, CDCl₃) δ 5.81 (dq, J=9.0, 1.3 Hz, 1H), 4.79 (ddd, J=9.1, 8.2, 4.6 Hz, 1H), 4.13 (d, J=17.1 Hz, 1H), 4.05 (d, J=17.1 Hz, 1H), 2.86 (dd, J=15.6, 8.1 Hz, 1H), 2.55 (dd, J=15.6, 4.6 Hz, 1H), 2.27 (d, J=1.3 Hz, 3H), 0.84 (s, 9H), 0.37 (s, 9H), 0.04 (s, 6H); ¹³C NMR (100 MHz, CDCl₃) δ 200.5, 165.4, 165.3, 161.1, 140.7, 134.2, 121.8, 66.9, 49.7, 43.7, 25.7, 24.0, 18.9, 18.0, −2.1, −4.6, −5.1; HRMS-ESI m/z calcd for C₂₀H₃₅BrNO₅Si₂ [M+H]⁺ 504.1232, found 504.1227.

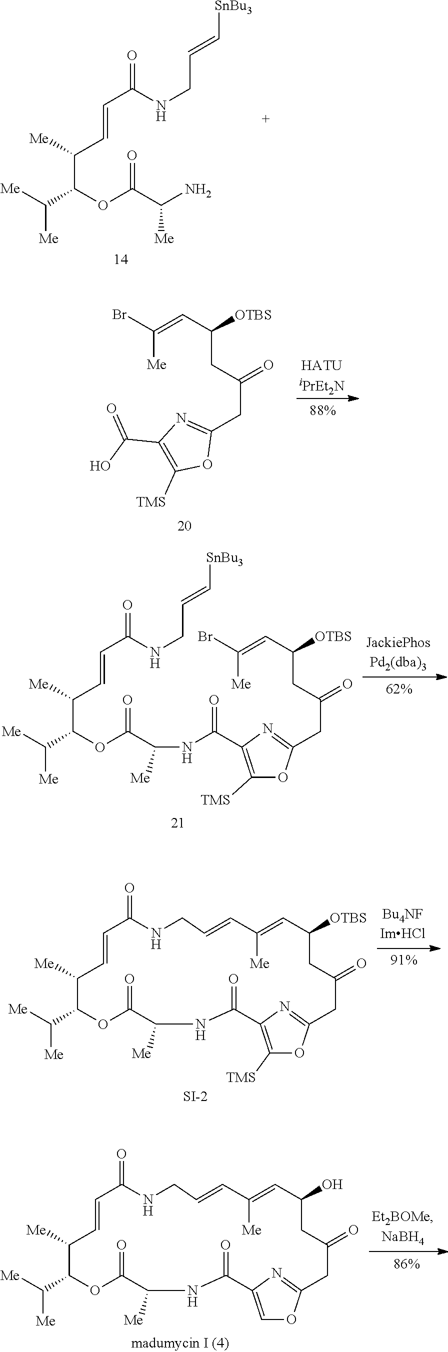

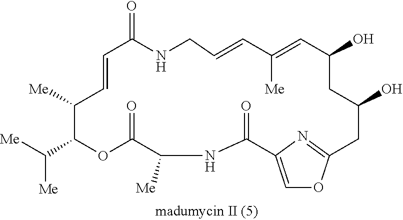 [SMILES: CC1=C\[C@@H](O)C[C@@H](O)Cc2nc(co2)C(=O)N[C@H](C)C(=O)O[C@H](C(C)C)[C@H](C)/C=C/C(=O)NC\C=C\1]

Stille Coupling Precursor 21:

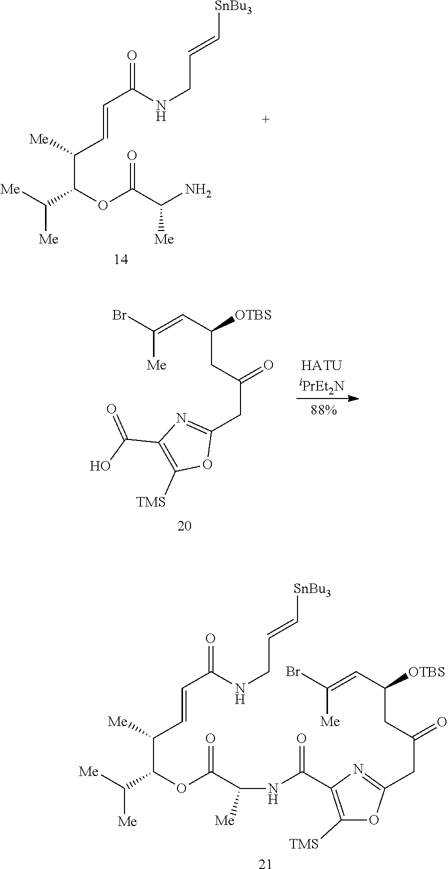

To a solution of amine 14 (0.7 g, 1.22 mmol), DIPEA (0.316 g, 0.427 mL, 2.45 mmol) and acid 20 (0.679 g, 1.35 mmol) in dry DCM (7.5 mL) was added HATU (0.582 g, 1.53 mmol) in one portion at RT. After stirring for 5 hours, the mixture was diluted with DCM, washed with water and brine, dried over Na₂SO₄ and concentrated under vacuum. The resulting residue was purified by flash chromatography (silica gel, EtOAc:Hexane=1:6 to 1:4) to afford Stille Coupling precursor 21 (1.14 g, 88%) as a light yellow slurry.

TLC (ethyl acetate:hexane=1:4): R_{f}=0.20 (UV); [α]²⁴_{D}=−26.5 (c=0.57, CH₂Cl₂); ¹HNMR (400 MHz, CDCl₃, rotamers) δ 7.55 (d, J=7.9 Hz, 0.1H), 7.39 (d, J=8.0 Hz, 0.8H), 7.11 (d, J=7.8 Hz, 0.1H), 6.71 (dd, J=15.4, 8.1, 1H), 6.17-6.06 (m, 1H), 5.97 (dt, J=19.1, 5.1 Hz, 1H), 5.89-5.78 (m, 2H), 5.67 (t, J=5.8 Hz, 1H), 4.87-4.64 (m, 3H), 4.07-3.95 (m, 2H), 3.93 (s, 2H), 2.84 (dd, J=15.3, 8.2, 1H), 2.70-2.60 (m, 1H), 2.58-2.48 (m, 1H), 2.29 (d, J=1.2 Hz, 3H), 1.97-1.81 (m, 1H), 1.55-1.40 (m, 9H), 1.35-1.23 (m, 6H), 1.07-0.99 (m, 3H), 0.92-0.82 (m, 30H), 0.35 (s, 9H), 0.48 (s, 6H); ¹³C NMR (100 MHz, CDCl₃, major rotamers) δ 200.9, 172.5, 165.2, 161.0, 160.9, 159.6, 145.0, 143.7, 143.4, 134.2, 130.3, 124.1, 121.8, 81.0, 67.0, 49.7, 47.8, 44.9, 44.1, 38.0, 29.8, 29.0, 27.2, 25.7, 24.0, 19.5, 18.7, 18.0, 16.6, 14.9, 13.7, 9.4, −2.0, −4.6, −5.1; HRMS-ESI m/z calcd for C₄₇H₈₄BrN₃NaO₇Si₂Sn [M+Na]⁺ 1080.3945, found 1080.3927.

OTBS-TMS-Madumycin I (SI-2):

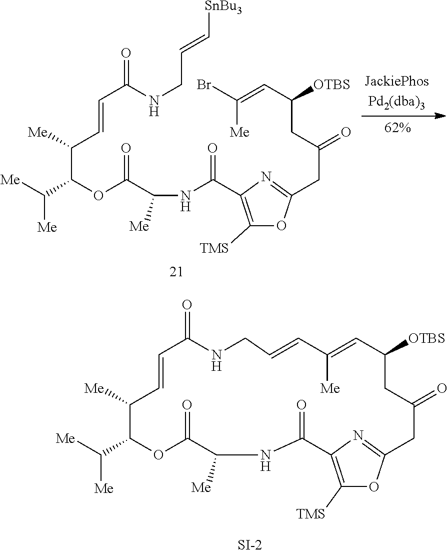

An oven-dry 100-mL round-bottom flask equipped with a stir bar was charged with Jackiephos (0.060 g, 0.075 mmol), Pd₂(dba)₃ (0.034 g, 0.037 mmol) and Stille Coupling precursor 21 (0.4 g, 0.378 mmol). The flask was evacuated and filled with nitrogen (this process was repeated 3 times total). Degassed toluene (32 mL) was added and the mixture was bubbled with Argon balloon for 30 min. Then the mixture was heated at 50° C. for 3 hours and the TLC showed that the SM was gone. The solvent was removed and the residue was purified by flash chromatography (EtOAc:Hexane=1:3 to 1:2) to afford OTBS-TMS-madumycin I (0.16 g, 62%) as a white foam.

TLC (ethyl acetate:hexane=1:2): R_{f}=0.20 (UV); [α]²⁴_{D}=−67.4 (c=1.0, CHCl₃); ¹H NMR (400 MHz, CDCl₃) δ 7.40 (d, J=8.4 Hz, 1H), 6.48 (dd, J=16.2, 5.1 Hz, 1H), 6.24-6.14 (m, 1H), 5.95-5.87 (m, 1H), 5.81 (dd, J=16.2, 1.8 Hz, 1H), 5.65 (ddd, J=15.7, 7.9, 3.6 Hz, 1H), 5.47 (d, J=9.0 Hz, 1H), 4.93 (td, J=8.8, 5.2 Hz, 1H), 4.79-4.70 (m, 2H), 4.52-4.38 (m, 1H), 3.91 (d, J=17.3 Hz, 1H), 3.73 (d, J=17.3 Hz, 1H), 3.62-3.49 (m, 1H), 2.96 (dd, J=14.5, 8.6 Hz, 1H), 2.81 (dd, J=14.5, 5.3 Hz, 1H), 2.78-2.70 (m, 1H), 2.02-1.90 (m, 1H), 1.70 (d, J=1.2 Hz, 3H), 1.38 (d, J=7.2 Hz, 3H), 1.08 (d, J=6.9 Hz, 3H), 0.96 (d, J=6.7 Hz, 3H), 0.91 (d, J=6.5 Hz, 3H), 0.85 (s, 9H), 0.34 (s, 9H), 0.04 (s, 3H), 0.02 (s, 3H); ¹³C NMR (100 MHz, CDCl₃) δ 201.3, 173.34, 166.6, 160.6, 160.4, 159.9, 144.3, 143.5, 134.9, 133.6, 133.4, 125.2, 124.2, 82.0, 66.2, 50.4, 47.1, 43.9, 41.0, 36.8, 29.3, 25.7, 19.8, 19.3, 18.6, 18.1, 13.0, 9.87, −2.0, −4.5, −5.0; HRMS-ESI m/z calcd for C₃₅H₅₈N₃O₇Si₂ [M+H]⁺ 688.3808, found 688.3811.

Madumycin I (4):

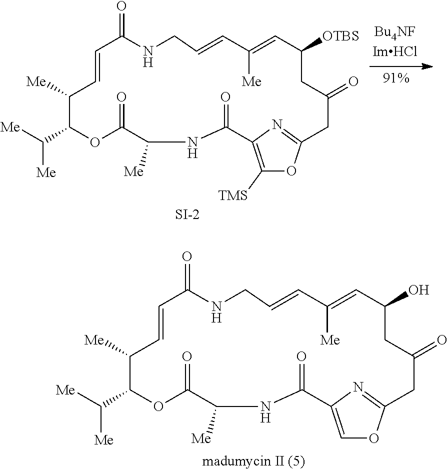 [SMILES: CC1=C\[C@@H](O[Si](C)(C)C(C)(C)C)CC(=O)Cc2nc(c([Si](C)(C)C)o2)C(=O)N[C@H](C)C(=O)O[C@H](C(C)C)[C@H](C)/C=C/C(=O)NC\C=C\1 ; "SI-2" ; CC1=C\[C@@H](O)CC(=O)Cc2nc(co2)C(=O)N[C@H](C)C(=O)O[C@H](C(C)C)[C@H](C)/C=C/C(=O)NC\C=C\1]

To a solution of SI-2 (0.105 g, 0.152 mmol) in dry THF (3 mL) was added 1 M TBAF (1.53 mL, 1.53 mmol) in THF which was charged with Im.HCl (0.159 g, 1.526 mmol). After stirring for 12 hour, the solvent was removed, diluted with DCM, washed with water and brine, dried over Na₂SO₄ and concentrated under vacuum. The resulting residue was purified by flashed chromatography (silica gel, MeOH:DCM=1:100 to 1:50) to afford madumycin I (0.07 g, 91%) as a white solid.

TLC (MeOH:DCM=1:25): R_{f}=0.20 (UV); [α]²⁴_{D}=−74.6 (c=0.64, CHCl₃); ¹H NMR (400 MHz, CDCl₃) δ 8.11 (s, 1H), 7.33 (d, J=7.9 Hz, 1H), 6.53 (dd, J=16.1, 5.5 Hz, 1H), 6.16 (d, J=15.7 Hz, 1H), 5.93 (dd, J=8.1, 4.3 Hz, 1H), 5.83 (dd, J=16.2, 1.7 Hz, 1H), 5.68 (ddd, J=15.7, 7.4, 3.7 Hz, 1H), 5.52 (d, J=9.0 Hz, 1H), 4.93 (ddd, J=8.9, 6.8, 5.7 Hz, 1H), 4.78 (dd, J=10.3, 2.0 Hz, 1H), 4.75-4.65 (m, 1H), 4.43-4.27 (m, 1H), 3.90 (d, J=16.8 Hz, 1H), 3.78 (d, J=16.7 Hz, 1H), 3.72-3.57 (m, 1H), 3.09 (dd, J=16.2, 6.9 Hz, 1H), 2.94 (dd, J=16.2, 5.7 Hz, 1H), 2.76 (ddt, J=7.2, 5.4, 1.8 Hz, 1H), 2.03-1.90 (m, 2H), 1.73 (d, J=1.2 Hz, 3H), 1.43 (d, J=7.1 Hz, 3H), 1.09 (d, J=6.8 Hz, 3H), 0.97 (d, J=6.8 Hz, 3H), 0.92 (d, J=6.5 Hz, 4H); ¹³C NMR (100 MHz, CDCl₃) δ 201.8, 172.8, 166.4, 159.6, 157.8, 144.6, 141.6, 136.1, 135.1, 134.6, 132.0, 125.6, 124.3, 82.2, 65.2, 48.7, 47.5, 43.38, 40.8, 36.8, 29.4, 19.7, 18.9, 18.6, 13.0, 10.1; HRMS-ESI m/z calcd for C₂₆H₃₆N₃O₇ [M+H]⁺ 502.2548, found 502.2546.

Madumycin II (5):

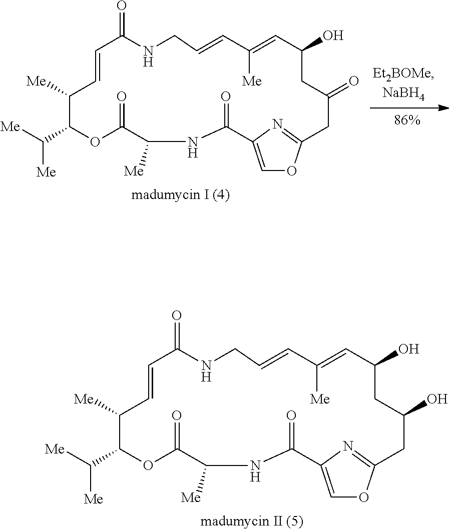 [SMILES: CC1=C\[C@@H](O)CC(=O)Cc2nc(co2)C(=O)N[C@H](C)C(=O)O[C@H](C(C)C)[C@H](C)/C=C/C(=O)NC\C=C\1 ; CC1=C\[C@@H](O)C[C@@H](O)Cc2nc(co2)C(=O)N[C@H](C)C(=O)O[C@H](C(C)C)[C@H](C)/C=C/C(=O)NC\C=C\1]

Madumycin I (4) was dried over azeotrope with toluene twice. To a solution of Madumycin I (4) (0.03 g, 0.059 mmol) in dry THF (2 mL) and dry MeOH (0.5 mL) was added 1 M diethyl(methoxy)borane (0.071 mL, 0.071 mmol) in THF at −78° C. under N₂. After stirring for 15 min, sodium borohydride (2.715 mg, 0.071 mmol) was added. After stirring for 3 hours, the reaction mixture was quenched with acetic acid (0.25 mL), diluted with EtOAc and warmed to RT. The organic mixture was washed with water and brine, dried over Na₂SO₄ and concentrated under vacuum. The resulting residue was purified by flashed chromatography (silica gel, MeOH:DCM=1:50 to 1:20) to afford Madumycin II (5) (0.026 g, 86%).

TLC (MeOH:DCM=1:10): R_{f}=0.30 (UV); [α]²⁴_{D}=−120.0 (c=1.0, MeOH); ¹H NMR (400 MHz, CDCl₃) δ 8.06 (s, 1H), 7.37 (d, J=8.3 Hz, 1H), 6.56 (dd, J=16.0, 5.6 Hz, 1H), 6.18 (d, J=15.7 Hz, 1H), 6.10-6.00 (m, 1H), 5.83 (dd, J=16.0, 1.7 Hz, 1H), 5.65 (ddd, J=15.7, 7.0, 3.7 Hz, 1H), 5.49 (d, J=8.9 Hz, 1H), 4.86-4.64 (m, 3H), 4.39-4.26 (m, 1H), 4.12 (dd, J=10.4, 5.3 Hz, 1H), 3.62 (ddd, J=16.9, 7.1, 4.3 Hz, 1H), 3.18 (brs, 1H) 2.98 (dd, J=16.2, 5.2 Hz, 1H), 2.86 (dd, J=16.1, 7.4 Hz, 1H), 2.78-2.68 (m, 1H), 2.00-1.81 (m, 3H), 1.76 (s, 3H), 1.41 (d, J=7.2 Hz, 3H), 1.09 (d, J=6.8 Hz, 3H), 0.95 (d, J=6.7 Hz, 3H), 0.89 (d, J=6.4 Hz, 3H); ¹³C NMR (100 MHz, CDCl₃) δ 172.6, 166.2, 162.0, 160.0, 145.1, 140.7, 135.5, 134.7, 134.5, 133.8, 124.8, 124.20, 82.1, 67.6, 67.4, 47.2, 43.0, 40.7, 36.8, 35.97, 29.4, 19.6, 19.0, 18.6, 13.0, 10.4; HRMS-ESI m/z calcd for C₂₆H₃₈N₃O₇ [M+H]⁺ 502.2704, found 502.2697.

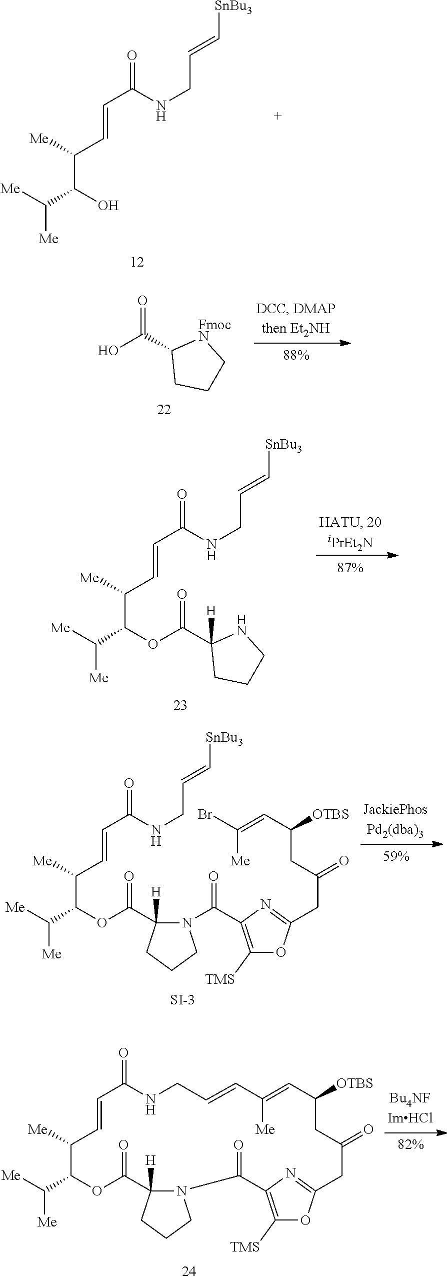

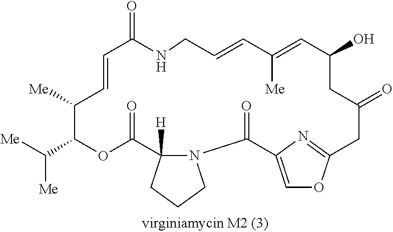 [SMILES: CC1=C\[C@@H](O)CC(=O)Cc2nc(co2)C(=O)N2CCC[C@@H]2C(=O)O[C@H](C(C)C)[C@H](C)/C=C/C(=O)NC\C=C\1]

Amine 23:

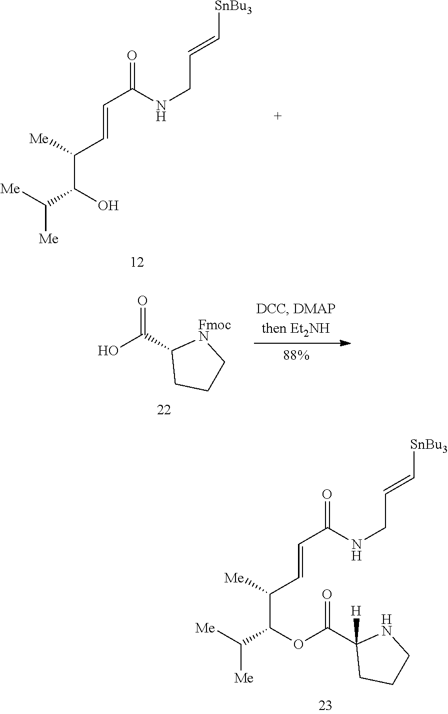

DCC (1.36 g, 6.60 mmol) was added to a stirred solution of 22 (2.0 g, 5.94 mmol), DMAP (0.107 g, 0.88 mmol) and 12 (2.2 g, 4.4 mmol) in dry CH₂Cl₂ (44 mL) in one portion at RT and the reaction mixture was stirred overnight. Diethylamine (22 mL) was added to this mixture. After stirring for additional 3 hours, the mixture was filtered over a pad of celite and the filter cake was washed with DCM. The solvent was removed under vacuum and the residue was purified by flash chromatography (silica gel, ammonia hydroxide:MeOH:DCM=0.2:1:100 to 0.2:1:50) to afford amine 23 (2.32 g, 88%) as colorless slurry.

TLC (MeOH:DCM=1:20): R_{f}=0.20 (UV); [α]²⁴_{D}=+13.9 (c=0.1, CHCl₃); ¹H NMR (400 MHz, CDCl₃) δ 6.70 (dd, J=15.4, 7.8 Hz, 1H), 6.11 (dt, J=19.0, 1.5 Hz, 1H), 5.96 (dt, J=19.0, 5.1 Hz, 1H), 5.82 (dd, J=15.4, 1.2 Hz, 1H), 5.59 (t, J=5.9 Hz, 1H), 4.81 (dd, J=6.9, 5.4 Hz, 1H), 4.03-3.89 (m, 2H), 3.76 (dd, J=8.5, 5.6 Hz, 1H), 3.07 (ddd, J=10.2, 7.4, 6.1 Hz, 1H), 2.89 (ddd, J=10.2, 7.1, 6.2 Hz, 1H), 2.65 (dtd, J=8.0, 6.8, 1.2 Hz, 1H), 2.13 (dtd, J=12.3, 8.1, 6.6 Hz, 1H), 2.07 (s, 1H), 1.94-1.79 (m, 2H), 1.80-1.65 (m, 2H), 1.56-1.39 (m, 6H), 1.35-1.21 (m, 6H), 1.04 (d, J=6.8 Hz, 3H), 0.95-0.80 (m, 21H); ¹³C NMR (100 MHz, CDCl₃) δ 175.3, 165.2, 145.2, 143.4, 130.4, 123.8, 80.3, 59.9, 46.9, 44.9, 38.2, 30.5, 29.8, 29.0, 27.2, 25.4, 19.6, 16.8, 14.7, 13.7, 9.4; HRMS-ESI m/z calcd for C₂₉H₅₅N₂O₃Sn [M+H]⁺ 599.3229, found 599.3219.

Stille Coupling precursor SI-3:

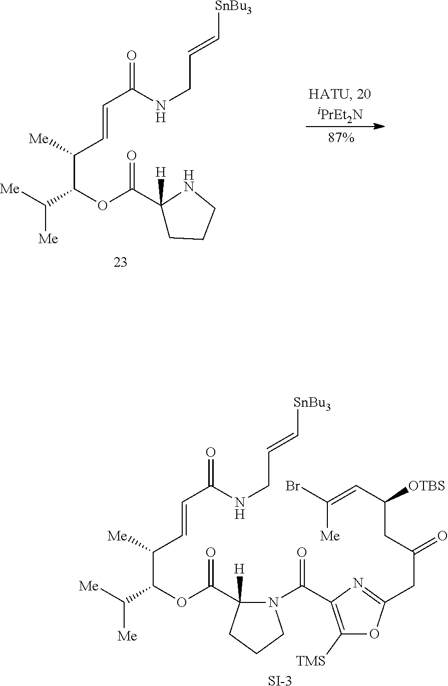

To a solution of DIPEA (0.194 g, 0.263 mL, 1.506 mmol), amine 23 (0.45 g, 0.753 mmol) and acid 20 (0.418 g, 0.828 mmol) in dry DCM (7.5 mL) was added HATU (0.357 g, 0.941 mmol) in one portion at RT. After stirring for 5 hours, the mixture was diluted with DCM, washed with water and brine, dried over Na₂SO₄ and concentrated under vacuum. The resulting residue was purified by flash chromatography (silica gel, EtOAc:Hexane=1:6 to 1:4) to afford Stille Coupling precursor SI-3 (0.71 g, 86.96%) as a white foam.

TLC (ethyl acetate:hexane=1:4): R_{f}=0.30 (UV); [α]²⁴_{D}=−10.7 (c=1.0, CHCl₃); NMR (400 MHz, CDCl₃, mixtures of retainers) δ 6.76-6.53 (m, 1H), 6.11 (dd, J=18.9, 1.6 Hz, 1H), 6.03-5.90 (m, 1H), 5.89-5.71 (m, 2H), 5.70-5.54 (m, 1H), 4.86-4.55 (m, 3H), 4.14-3.82 (m, 5H), 3.81-3.61 (m, 1H), 2.90-2.75 (m, 1H), 2.68-2.45 (m, 2H), 2.35-2.22 (m, 4H), 2.09-1.75 (m, 4H), 1.52-1.42 (m, J=8.3, 6.0 Hz, 6H), 1.35-1.22 (dq, J=13.3, 6.6, 6.0 Hz, 6H), 1.08-0.99 (m, 3H), 0.99-0.78 (m, 30H), 0.37-0.26 (m, 9H), 0.11-0.01 (m, 6H); ¹³C NMR (100 MHz, CDCl₃, mixtures of retainers) δ 201.1, 200.7, 172.34 165.4, 165.1, 163.2, 162.5, 161.5, 159.1, 145.4, 145.2, 145.1, 143.4, 143.3, 134.2, 130.4, 130.2, 123.9, 123.8, 121.8, 80.8, 80.4, 67.0, 66.9, 60.5, 59.9, 49.6, 48.8, 47.1, 44.91, 44.86, 44.2, 44.0, 38.4, 38.1, 31.6, 29.9, 29.8, 29.7, 29.1, 29.0, 28.9, 27.5, 27.2, 27.0, 25.69, 25.67, 25.6, 25.2, 24.00, 23.99, 21.5, 19.7, 19.5, 18.0, 17.0, 16.8, 14.9, 14.6, 13.7, 11.14, 11.06, 9.4, 7.8, 7.7, −1.77, −1.79, −4.57, −5.13, −5.15; HRMS-ESI m/z calcd for C₄₉H₈₇BrN₃O₇Si₂Sn [M+H]+ 1084.4282, found 1084.4275.

OTBS-TMS-Virginiamycin M2:

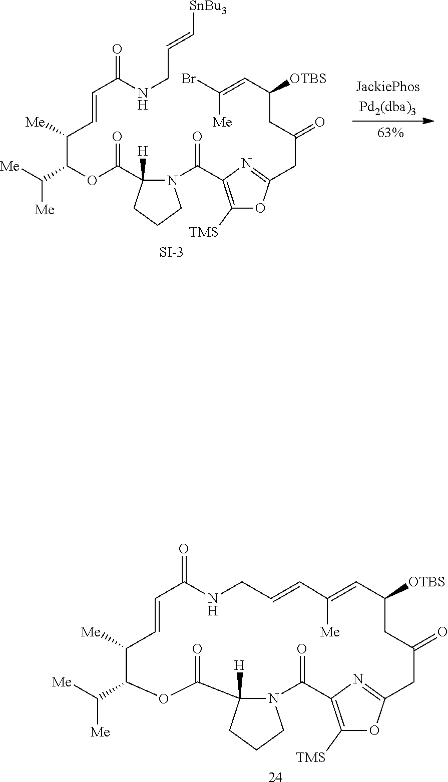

An oven-dry 100-mL round-bottom flask equipped with a stir bar was charged with Jackiephos (0.015 g, 0.019 mmol), SI-3 (0.108 g, 0.099 mmol) and Pd₂(dba)₃ (9.123 mg, 9.962 μmol). The flask was evacuated and filled with nitrogen (this process was repeated 3 times total). Degassed toluene (50 mL) was added and the mixture was bubbled with Argon balloon for 30 min. Then the mixture was heated at 50° C. for 3 hours and the TLC showed that the SM was gone. The solvent was removed and the residue was purified by flash chromatography (EtOAC:Hexane=1:2.5 to 1:2) to afford OTBS-TMS-virginiamycin M2 (0.045 g, 63.25%) as a white foam.

TLC (ethyl acetate:hexane=1:2): R_{f}=0.20 (UV); [α]²⁴_{D}=−57.1 (c=1.0, CHCl₃); ¹H NMR (400 MHz, CDCl₃) δ 6.49 (dd, J=16.3, 4.2 Hz, 1H), 6.19-6.10 (m, 1H), 6.07 (dd, J=9.2, 3.2 Hz, 1H), 5.77 (dd, J=16.4, 2.0 Hz, 1H), 5.57 (ddd, J=15.5, 9.4, 4.2 Hz, 1H), 5.42 (d, J=8.9 Hz, 1H), 5.00 (ddd, J=8.9, 7.0, 5.9 Hz, 1H), 4.85-4.72 (m, 2H), 4.57-4.43 (m, 1H), 3.89 (d, J=17.2 Hz, 1H), 3.78-3.69 (m, 3H), 3.39 (ddd, J=14.8, 9.5, 3.3 Hz, 1H), 2.92 (dd, J=15.9, 7.0 Hz, 1H), 2.79-2.68 (m, 2H), 2.18-2.04 (m, 1H), 1.90 (dddd, J=24.9, 15.9, 11.3, 6.8 Hz, 3H), 1.77-1.68 (m, 1H), 1.66 (d, J=1.2 Hz, 3H), 1.08 (d, J=6.9 Hz, 3H), 0.99 (d, J=6.5 Hz, 3H), 0.94 (d, J=6.8 Hz, 3H), 0.85 (s, 9H), 0.30 (s, 9H), 0.05 (s, 3H), 0.02 (s, 3H); ¹³C NMR (100 MHz, CDCl₃) δ 201.0, 172.1, 166.4, 161.8, 161.3, 159.6, 145.1, 144.8, 136.7, 134.7, 132.4, 124.9, 123.7, 81.1, 65.4, 58.7, 50.6, 48.4, 43.7, 41.3, 36.7, 29.3, 28.2, 25.7, 24.8, 19.9, 18.6, 18.1, 12.67, 9.9, −1.8, −4.5, −5.0; HRMS-ESI m/z calcd for C₃₇H₆₀N₃O₇Si₂ [M+H]⁺ 714.3964, found 714.3968.

Virginiamycin M2:

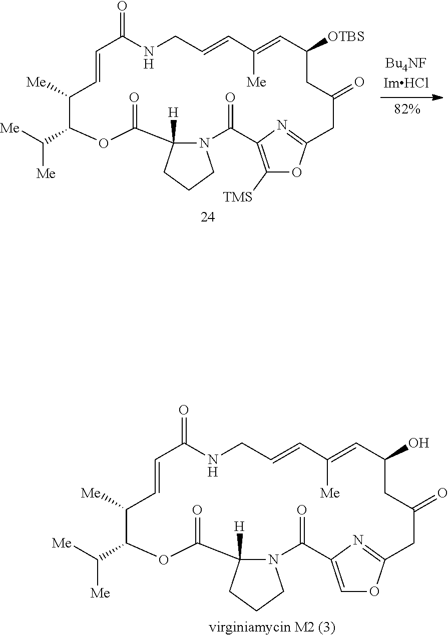

A solution of 24 (0.058 g, 0.081 mmol) in dry THF (2 mL) was treated with TBAF (0.812 mL, 0.812 mmol) which was charged with Im.HCl (0.084 g, 0.812 mmol) in advance. After stirring for 12 hour, the solvent was removed, diluted with DCM, washed with water and brine, dried over Na₂SO₄, concentrated under vacuum and the residue was purified by flash chromatography (MeOH:DCM=1:40) to afford virginiamycin M2 (0.035 g, 81.67%) as a white solid.

TLC (MeOH:DCM=1:20): R_{f}=0.30 (UV); [α]²⁵_{D}=−67.4 (c=0.3, CH₂Cl₂); ¹H NMR (400 MHz, Chloroform-d) δ 8.08 (s, 1H), 6.47 (dd, J=16.4, 5.0 Hz, 1H), 6.39 (dd, J=9.0, 3.7 Hz, 1H), 6.17-6.07 (m, 1H), 5.78 (dd, J=16.4, 1.9 Hz, 1H), 5.69 (ddd, J=15.6, 9.2, 4.6 Hz, 1H), 5.41 (d, J=8.8 Hz, 1H), 4.90 (dt, J=8.9, 5.6 Hz, 1H), 4.73 (dd, J=10.1, 2.0 Hz, 1H), 4.70 (dd, J=8.9, 3.2 Hz, 1H), 4.45 (ddd, J=13.9, 8.9, 4.6 Hz, 1H), 3.97 (dt, J=11.1, 7.3 Hz, 1H), 3.82 (s, 2H), 3.79-3.70 (m, 1H), 3.39 (ddd, J=14.0, 9.2, 3.6 Hz, 1H), 3.05 (dd, J=17.0, 6.0 Hz, 1H), 2.89 (dd, J=17.0, 5.2 Hz, 1H), 2.74 (ddt, J=6.9, 4.9, 2.0 Hz, 1H), 2.60 (br s, 1H), 2.24-2.08 (m, 1H), 2.01-1.88 (m, 3H), 1.88-1.75 (m, 1H), 1.71 (d, J=1.2 Hz, 3H), 1.03 (d, J=6.9 Hz, 3H), 0.98 (d, J=6.5 Hz, 3H), 0.95 (d, J=6.8 Hz, 3H); ¹³C NMR (100 MHz, CDCl₃) δ 202.3, 171.7, 166.7, 160.2, 156.8, 144.4, 144.0, 137.1, 136.9, 134.4, 132.5, 125.4, 124.1, 81.4, 65.2, 59.7, 48.8, 48.4, 43.2, 41.0, 36.7, 29.5, 28.4, 25.1, 19.75, 18.7, 12.7, 10.3; HRMS-ESI m/z calcd for C₂₈H₃₈N₃O₇ [M+H]⁺ 528.2704, found 528.2703.

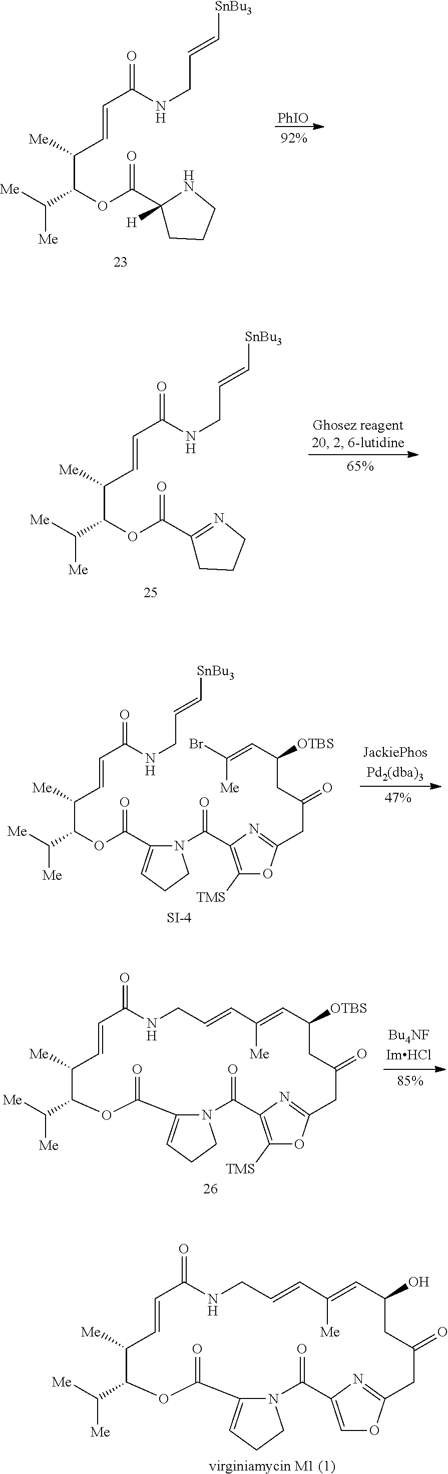

Imine 25:

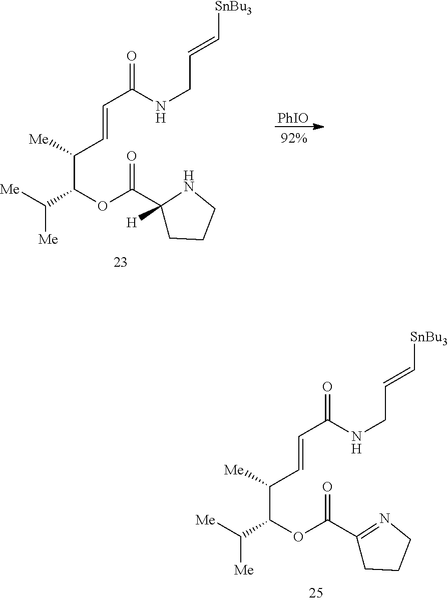

Iodosylbenzene (0.94 g, 4.27 mmol) was added to a stirred solution of amine 23 (2.32 g, 3.88 mmol) in dry DCM (39 mL) in one portion at RT and the reaction mixture was stirred for 30 min. The solvent was removed under vacuum and the residue was purified by flash chromatography (silica gel, EtOAc:Hexane=1:1.5) to afford imine 25 (2.12 g, 91.69%) as colorless oil.

TLC (ethyl acetate:hexane=1:1.5): R_{f}=0.20 (UV); [α]²⁴_{D}=−−0.9 (c=1.0, CHCl₃); ¹H NMR (400 MHz, CDCl₃) δ 6.72 (dd, J=15.4, 8.2 Hz, 1H), 6.11 (dt, J=18.9, 1.5 Hz, 1H), 5.96 (dt, J=19.1, 5.1 Hz, 1H), 5.86 (dd, J=15.4, 1.0 Hz, 1H), 5.60 (t, J=5.9 Hz, 1H), 4.95 (dd, J=7.6, 4.7 Hz, 1H), 4.11 (tt, J=7.7, 2.6 Hz, 2H), 4.05-3.85 (m, 2H), 2.82 (tt, J=8.4, 2.5 Hz, 2H), 2.77-2.67 (m, 1H), 2.04-1.91 (m, 3H), 1.59-1.35 (m, 6H), 1.29 (h, J=7.3 Hz, 6H), 1.06 (d, J=6.8 Hz, 3H), 0.97-0.81 (m, 21H); ¹³C NMR (100 MHz, CDCl₃) δ 168.3, 165.2, 162.8, 144.8, 143.3, 130.4, 124.1, 81.5, 62.6, 44.9, 38.3, 35.6, 29.9, 29.01, 27.2, 22.04, 19.7, 16.4, 15.5, 13.66, 9.4; HRMS-ESI m/z calcd for C₂₉H₅₂N₂NaO₃Sn [M+Na]+ 615.2888, found 615.2884.

Stille Coupling precursor SI-4:

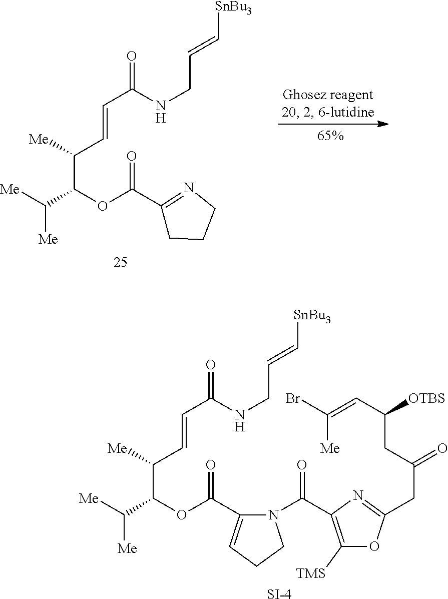

To a solution of acid 20 (3.177 g, 6.297 mmol) in DCM (31 mL) was added Ghosez reagent (0.875 g, 0.866 mL, 6.549 mmol) dropwise at RT under N₂. After stirring 2 hours, to a solution of imine 25 (3.0 g, 5.04 mmol), 2,6-lutidine (1.35 g, 1.47 mL, 12.6 mmol) in DCM (62 mL) was added the above acid chloride solution at RT under N₂. After stirring for overnight, the reaction was filtered over a pad of celite and concentrated under vacuum. The resulting residue was purified by flash chromatography (EtOAC:Hexane=1:5 to 1:3) to afford Stille Coupling precursor SI-4 (3.52 g, 65%) as yellow sticky solid.

TLC (ethyl acetate:hexane=1:3): R_{f}=0.20 (UV); [α]²⁴_{D}=−22.8 (c=1.0, CHCl₃); ¹H NMR (400 MHz, CDCl₃) δ 6.73 (dd, J=15.4, 8.4 Hz, 1H), 6.16-6.04 (m, 2H), 5.96 (dt, J=19.0, 5.0 Hz, 1H), 5.88-5.82 (m, 1H), 5.81 (dq, J=9.1, 1.3 Hz, 1H), 5.63 (t, J=5.9 Hz, 1H), 4.86 (dd, J=7.7, 4.6 Hz, 1H), 4.78 (ddd, J=9.2, 8.1, 4.8 Hz, 1H), 4.29 (t, J=8.5 Hz, 2H), 4.07-3.97 (m, 1H), 3.97-3.90 (m, 1H), 3.89 (s, 2H), 2.83 (dd, J=15.4, 8.0 Hz, 1H), 2.78-2.61 (m, 3H), 2.52 (dd, J=15.5, 4.8 Hz, 1H), 2.32-2.23 (m, 3H), 1.90 (td, J=6.8, 4.5 Hz, 1H), 1.53-1.41 (m, 6H), 1.35-1.23 (m, 6H), 1.08 (d, J=6.7 Hz, 3H), 0.96-0.80 (m, 30H), 0.36-0.28 (m, J=1.5 Hz, 9H), 0.04 (s, 6H); ¹³C NMR (100 MHz, CDCl₂, 50° C., major rotamer) δ 200.2, 165.6, 163.5, 161.7, 161.2, 159.4, 145.2, 143.9, 138.4, 135.2, 134.5, 130.5, 124.3, 121.9, 121.8, 81.3, 67.1, 50.8, 50.1, 45.0, 44.0, 38.9, 30.3, 29.6, 29.1, 27.2, 25.8, 24.0, 19.8, 18.1, 16.8, 15.6, 13.5, 9.7, −1.8, −4.5, −5.0; HRMS-ESI m/z calcd for C₄₉H₈₄BrN₃NaO₇Si₂Sn [M+Na]⁺ 1104.3945, found 1104.3926.

OTBS-TMS-Virginiamycin M1 (26):

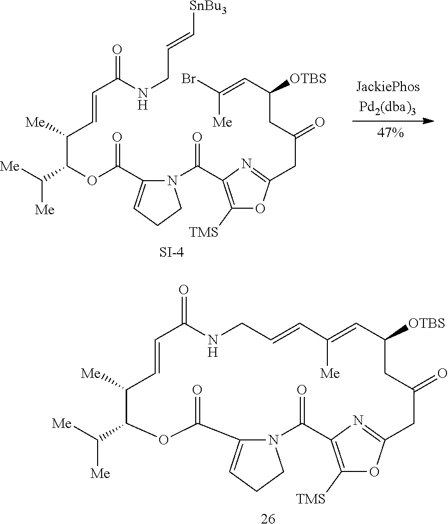

An oven-dry 100-mL round-bottom flask equipped with a stir bar was charged with Jackiephos (0.015 g, 0.019 mmol), SI-3 (0.108 g, 0.099 mmol) and Pd₂(dba)₃ (9.123 mg, 9.962 μmol). The flask was evacuated and filled with nitrogen (this process was repeated 3 times total). Degassed toluene (50 mL) was added and the mixture was bubbled with Argon balloon for 30 min. Then the mixture was heated at 50° C. for 3 hours and the TLC showed that the SM was gone. The solvent was removed and the residue was purified by flash chromatography (EtOAC:Hexane=1:2.5 to 1:2) to afford OTBS-TMS-virginiamycin M2 (0.045 g, 63.25%) as a white foam.

TLC (ethyl acetate:hexane=1:1.5): R_{f}=0.20 (UV); [α]²⁴_{D}=−108.84 (c=1.0, CHCl₃); ¹H NMR (400 MHz, CDCl₂, 50° C.) δ 6.81 (br s, 1H), 6.63 (dd, J=16.2, 7.0 Hz, 1H), 6.05-5.92 (m, 2H), 5.84 (d, J=15.7 Hz, 1H), 5.49 (ddd, J=16.0, 7.0, 4.7 Hz, 1H), 5.12 (d, J=8.7 Hz, 1H), 4.96-4.83 (m, 2H), 4.21-3.98 (m, 3H), 3.92 (dd, J=14.3, 2.7 Hz, 1H), 3.88-3.71 (m, 1H), 3.66 (d, J=14.0 Hz, 1H), 3.39-3.20 (m, 1H), 2.89-2.53 (m, 4H), 2.08-1.90 (m, 1H), 1.59 (s, 3H), 1.13 (d, J=6.9 Hz, 3H), 1.03-0.94 (m, 6H), 0.88 (s, 9H), 0.31 (s, 9H), 0.04 (s, 3H), 0.01 (s, 3H); ¹³C NMR (100 MHz, CDCl₃, 50° C.) δ 199.9, 167.4, 161.6, 161.1, 160.0, 159.0, 145.1, 143.0, 137.8, 134.7, 133.3, 132.8, 125.5, 125.2, 121.2, 81.8, 66.3, 50.6, 50.23, 44.6, 41.1, 37.9, 30.1, 29.6, 25.9, 19.5, 19.1, 18.1, 13.0, 12.1, −1.7, −4.4, −4.9; HRMS-ESI m/z calcd for C₃₇H₅₈N₃O₇Si₂ [M+H]+ 712.3808, found 712.3810.

Virginiamycin M1:

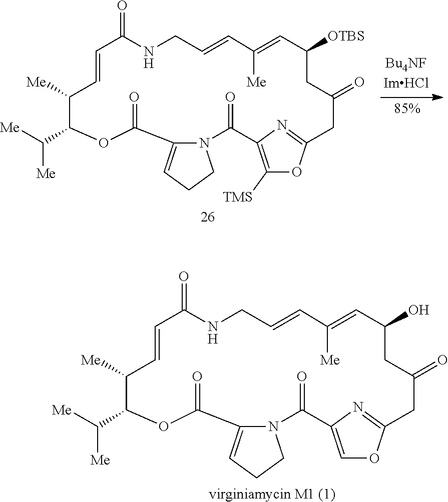

A solution of 26 (0.058 g, 0.081 mmol) in dry THF (2 mL) was treated with TBAF (0.812 mL, 0.812 mmol) which was charged with Im.HCl (0.084 g, 0.812 mmol) in advance. After stirring for 12 hour, the solvent was removed, diluted with DCM, washed with water and brine, dried over Na₂SO₄, concentrated under vacuum and the residue was purified by flash chromatography (MeOH:DCM=1:40) to afford virginiamycin M1 (0.035 g, 81.67%) as a white solid.

TLC (MeOH:DCM=1:20): R_{f}=0.20 (UV); [α]²⁵_{D}=−176.4 (c=0.5, EtOH); ¹H NMR (400 MHz, CDCl₃) δ 7.9 (s, 1H), 7.60 (t, J=5.7 Hz, 1H), 6.59 (dd, J=16.3, 7.4 Hz, 1H), 6.14 (t, J=2.9 Hz, 1H), 6.00 (dd, J=16.3, 1.1 Hz, 1H), 5.87-5.78 (m, 1H), 5.57 (ddd, J=15.9, 6.0, 3.6 Hz, 1H), 4.97-4.87 (m, 2H), 4.81 (td, J=9.4, 3.6 Hz, 1H), 4.38-4.28 (m, 2H), 4.21 (dt, J=18.4, 6.4 Hz, 1H), 3.92 (d, J=13.3 Hz, 2H), 3.71 (d, J=13.3 Hz, 1H), 3.15 (dd, J=13.9, 9.5 Hz, 1H), 2.90 (dtd, J=17.6, 9.8, 2.5 Hz, 1H), 2.79-2.64 (m, 3H), 2.02 (dp, J=10.0, 6.4 Hz, 2H), 1.56 (s, 3H), 1.11 (d, J=6.9 Hz, 3H), 0.98 (d, J=7.0 Hz, 3H), 0.95 (s, 3H); ¹³C NMR (100 MHz, CDCl₃) δ 200.9, 167.5, 160.9, 159.9, 155.9, 145.5, 143.0, 137.0, 135.9, 134.7, 133.5, 130.7, 126.1, 125.2, 122.9, 81.3, 66.0, 50.4, 47.4, 45.7, 40.4, 37.6, 30.1, 29.9, 19.5, 18.8, 12.7, 12.1; HRMS-ESI m/z calcd for C₂₈H₃₆N₃O₇ [M+H]+ 526.2548, found 526.2547.

Scheme converting dalfopristin to virginiamycin M1:

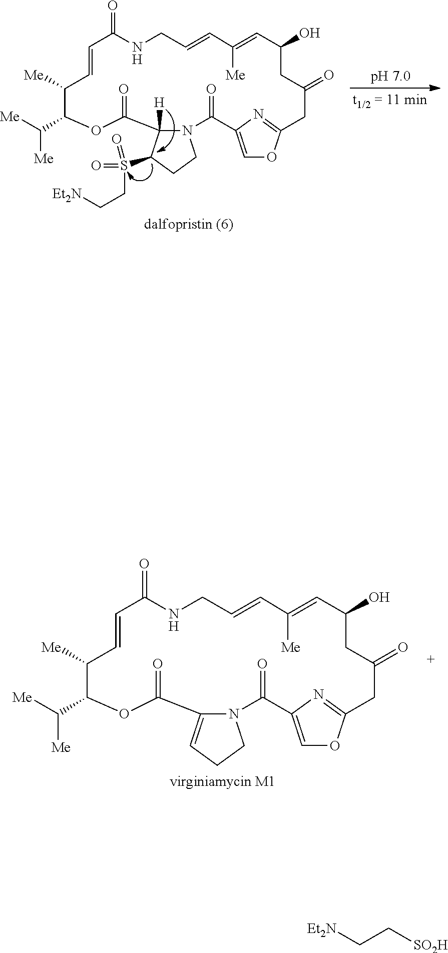

A. Synthesis of the demethylated analogue

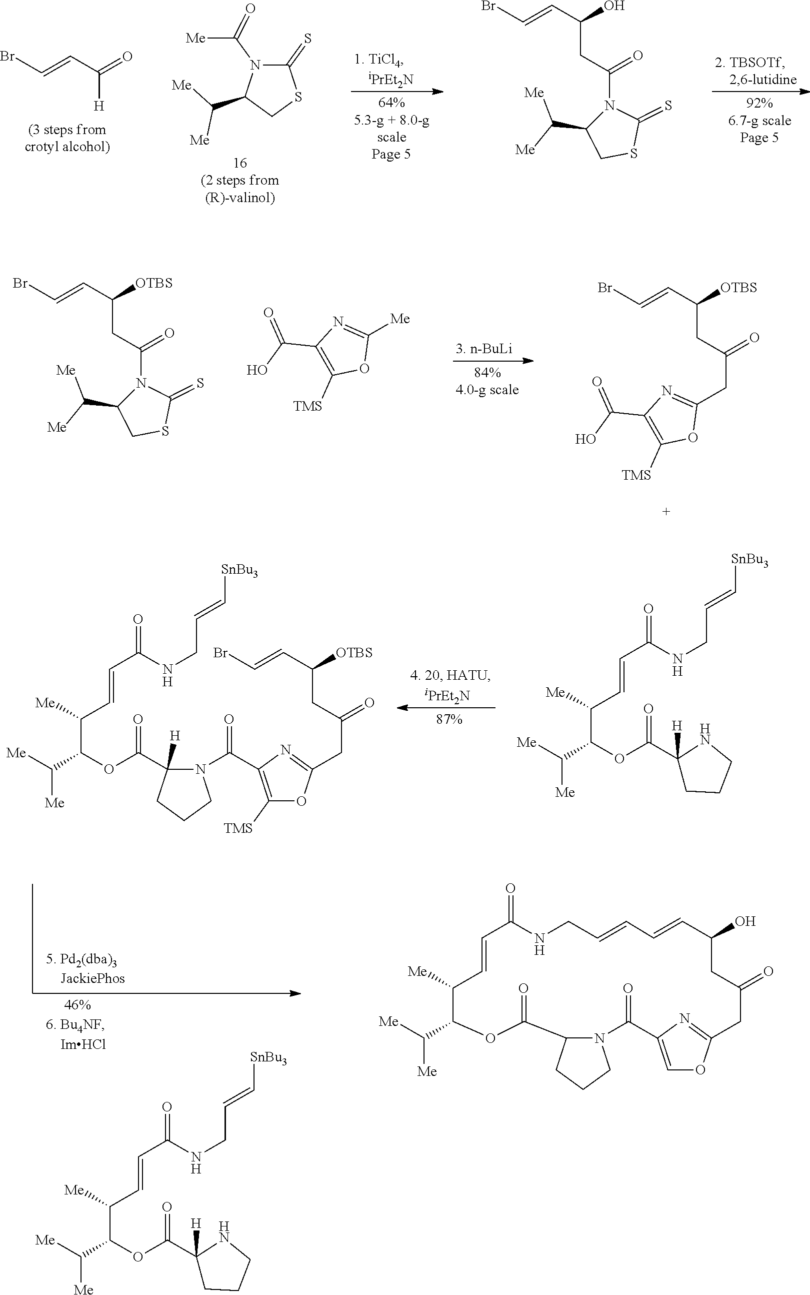

B. Synthesis of the Thioxazole Analogues

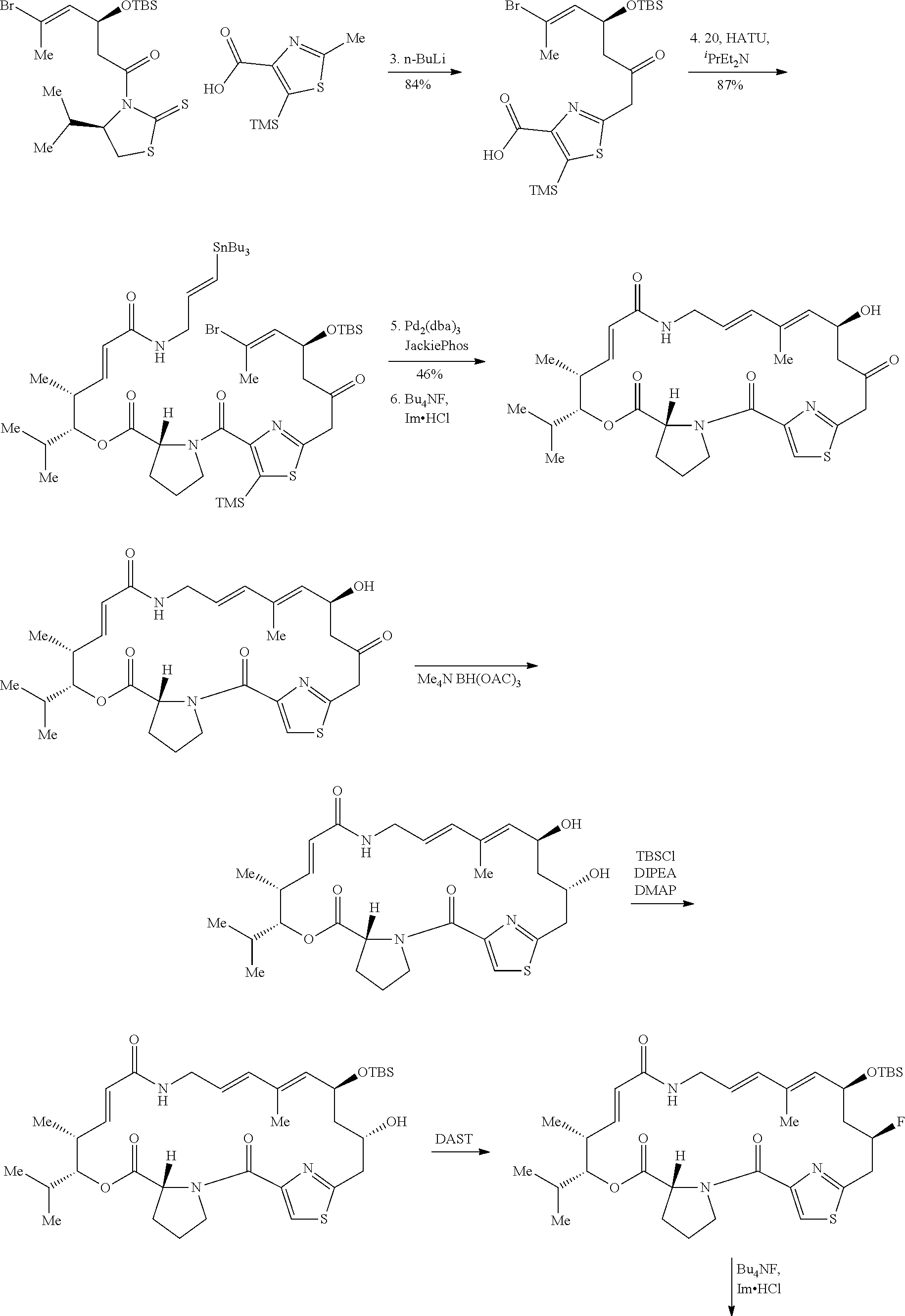

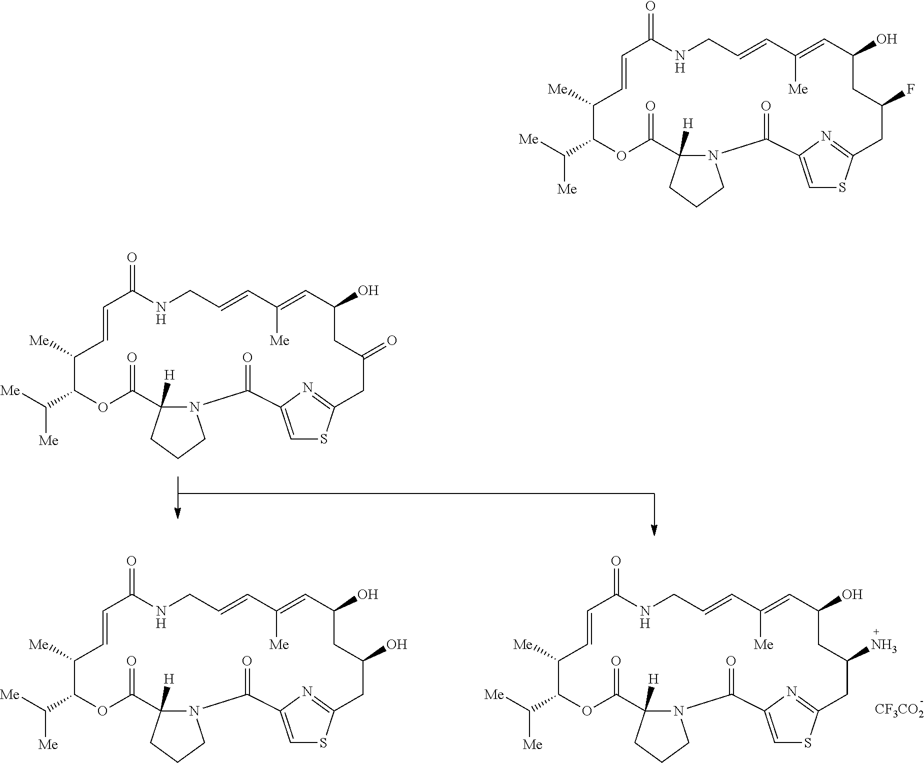

Syntheses of Analogues from (S)-Aldehyde

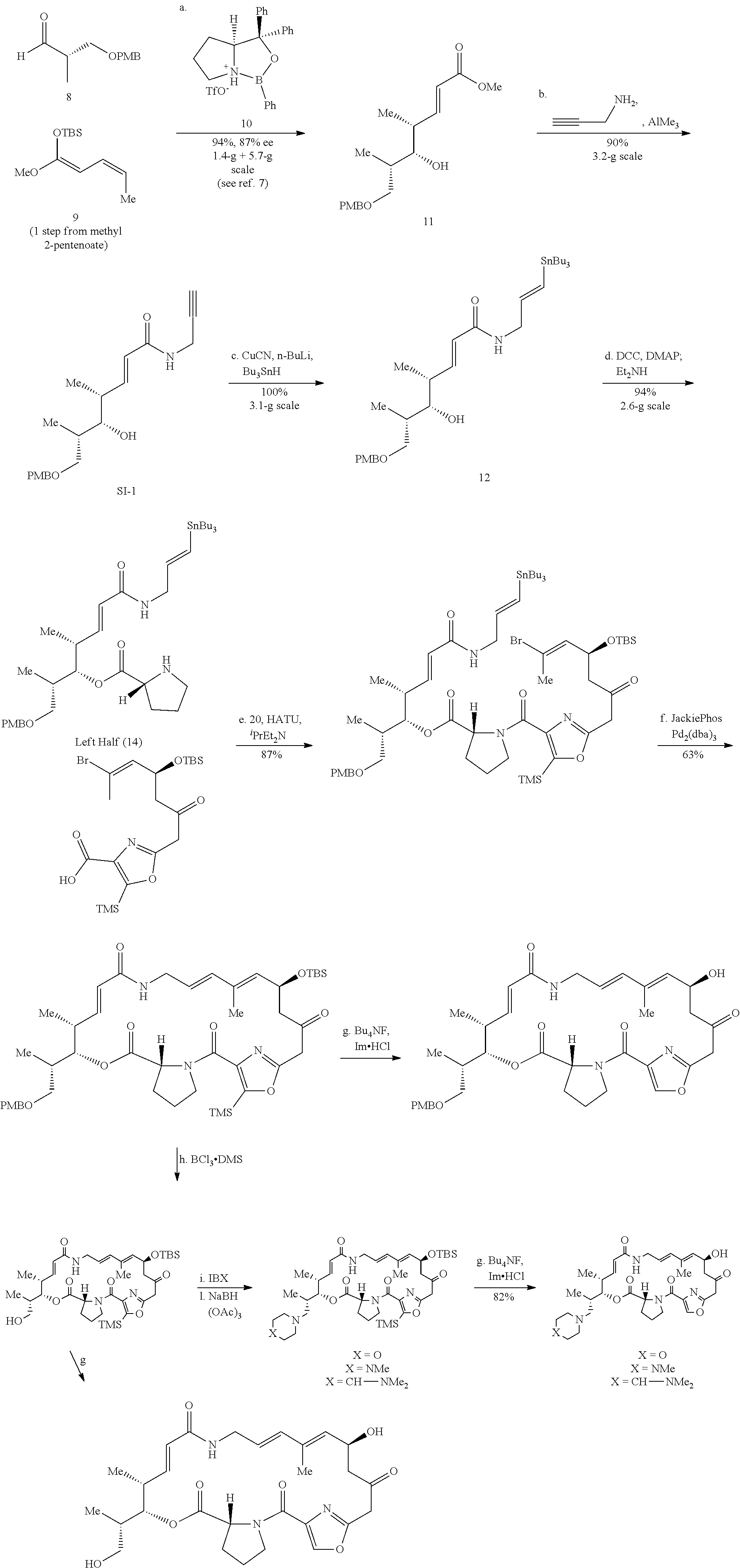

Route to Key Intermediate from (R)-Aldehyde:

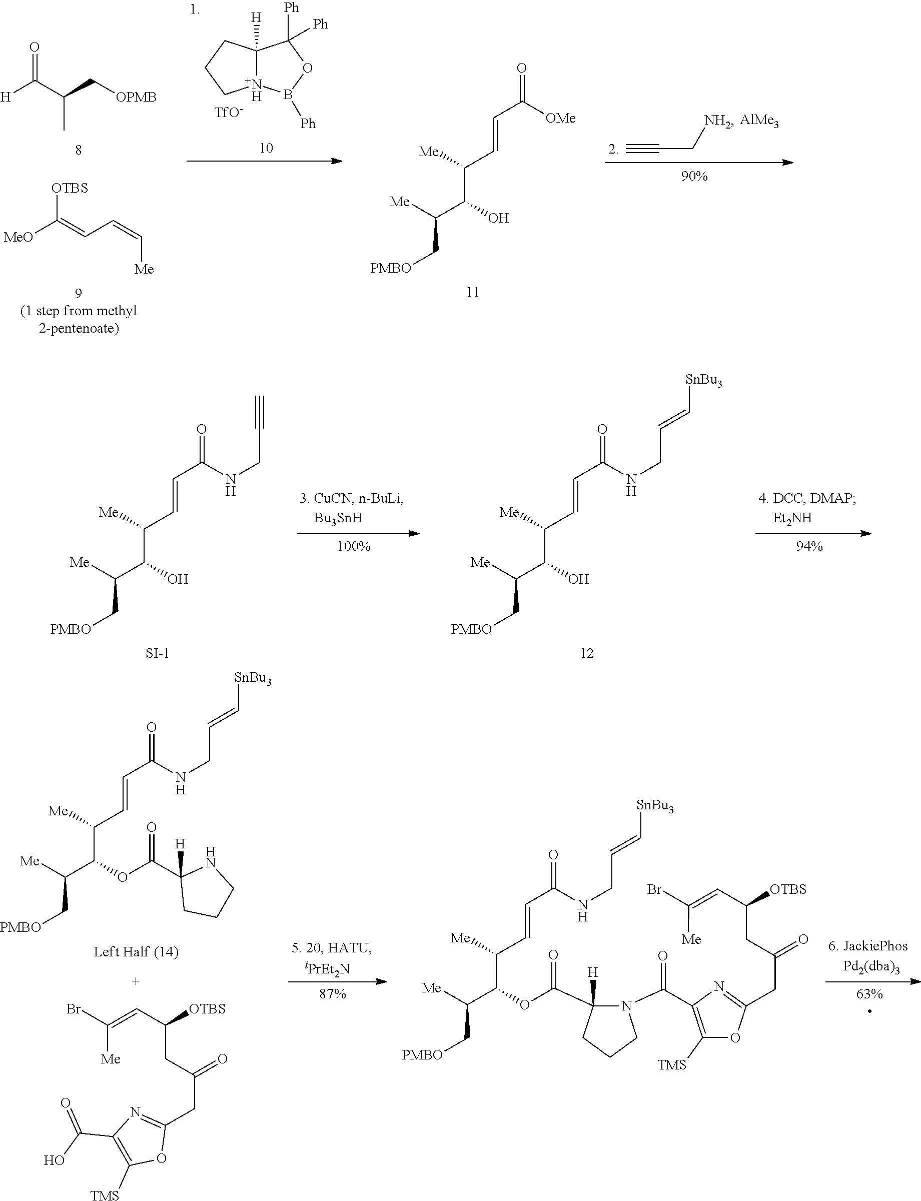

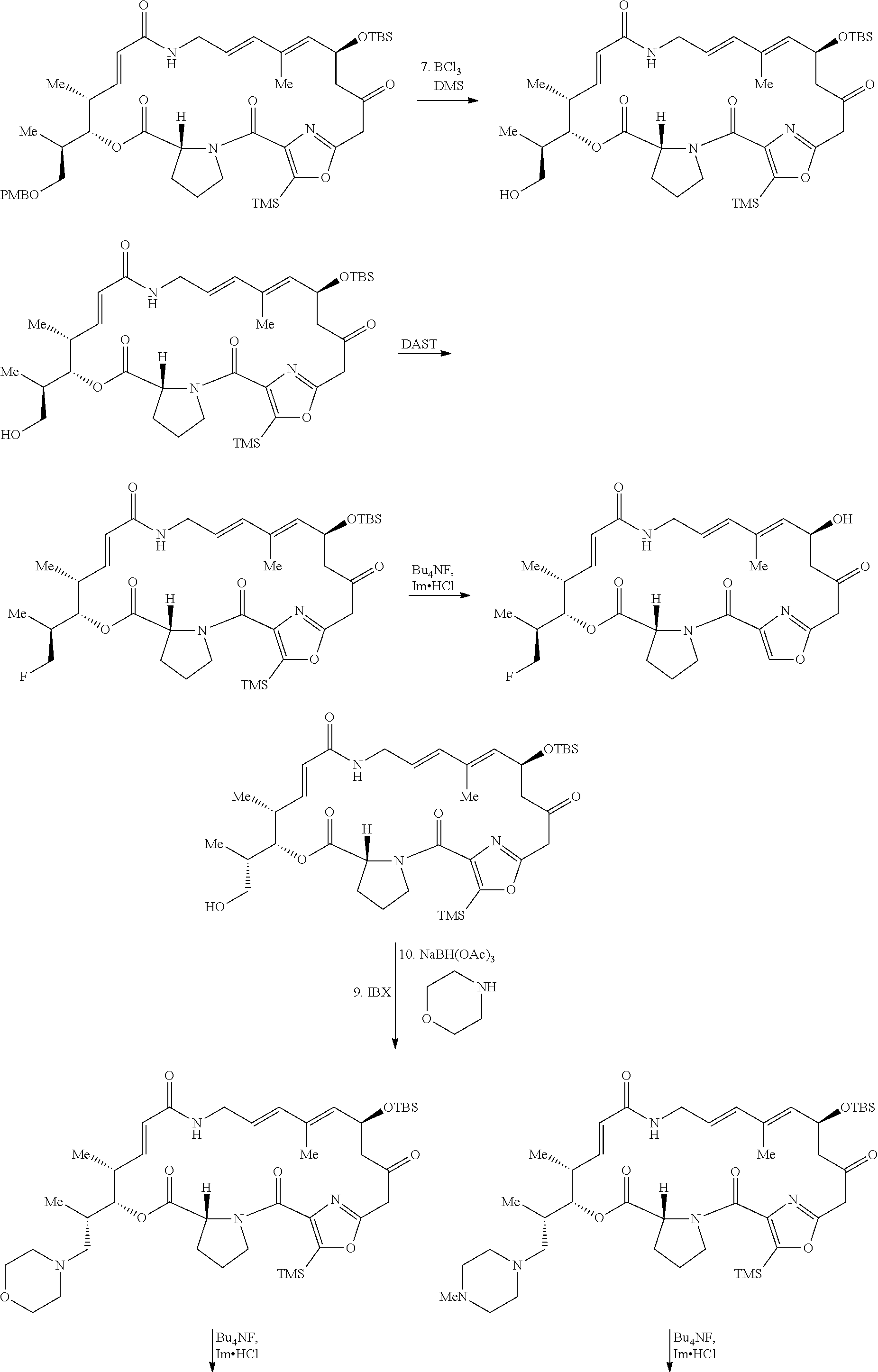

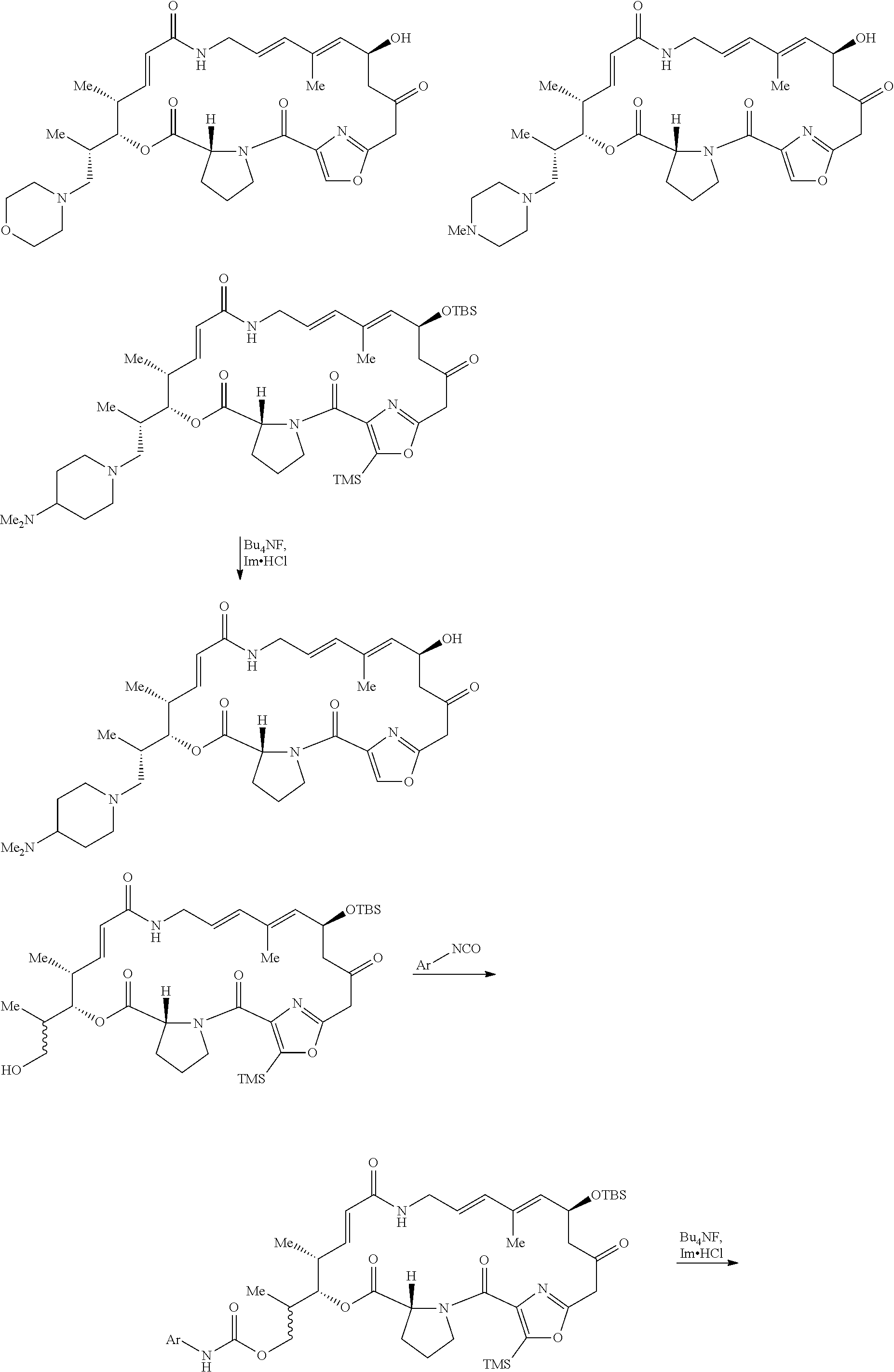

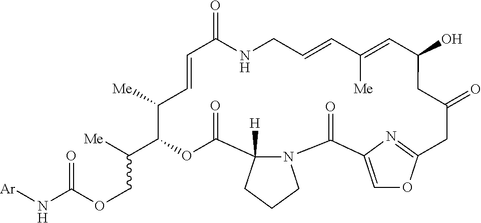

Amino Acid Analogues

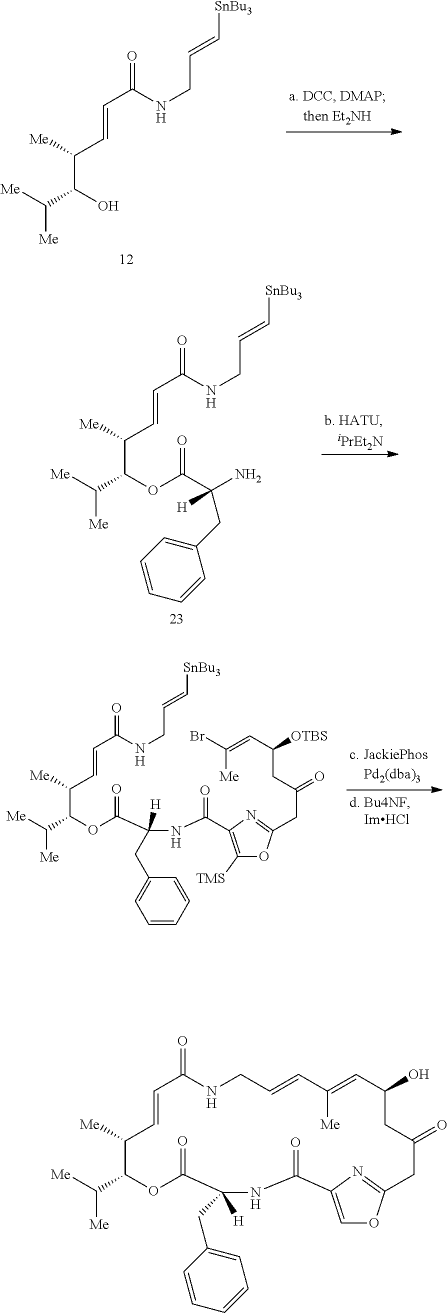

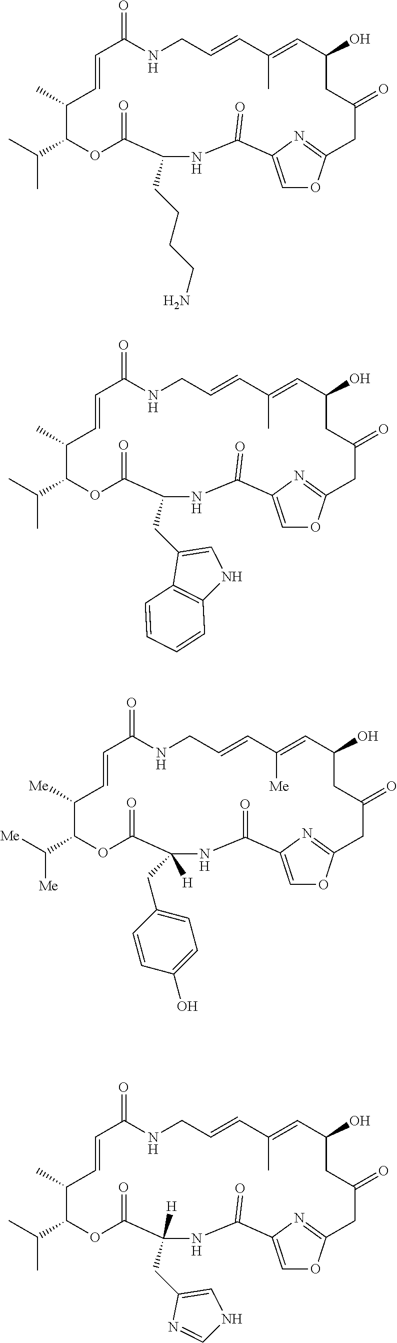

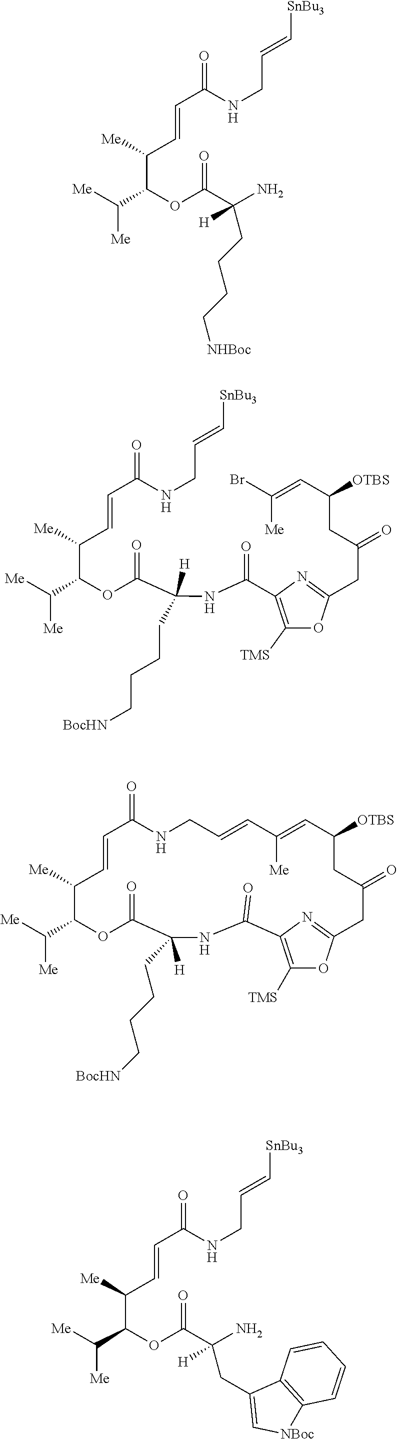

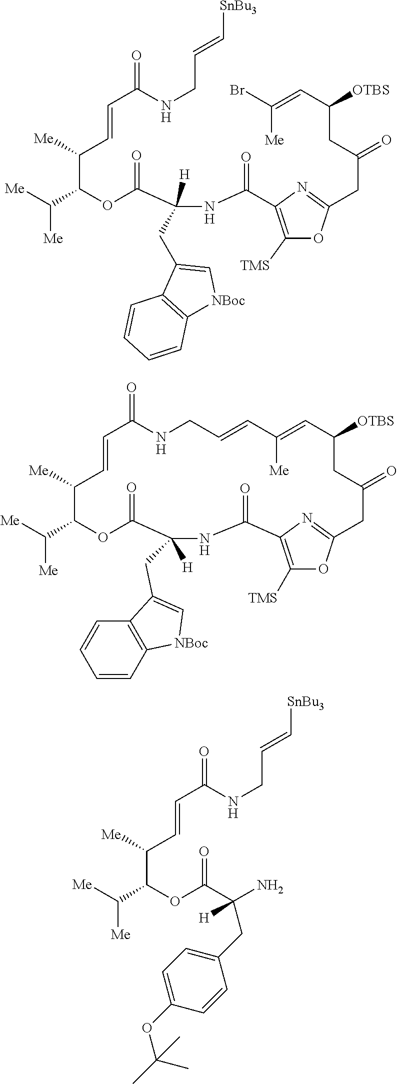

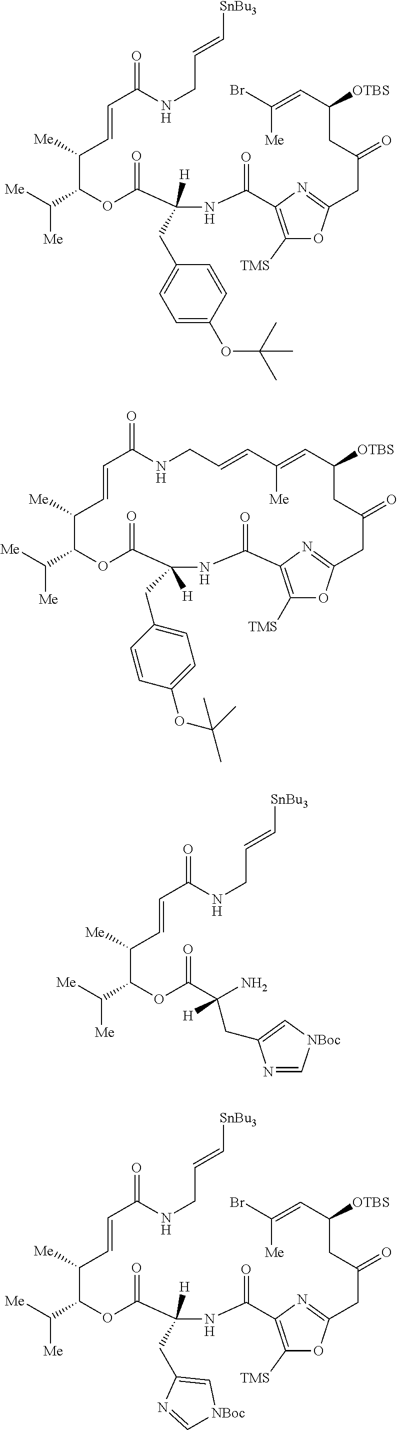

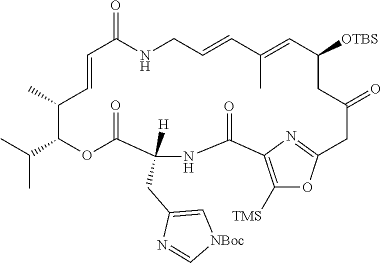

Carbamate Analogues

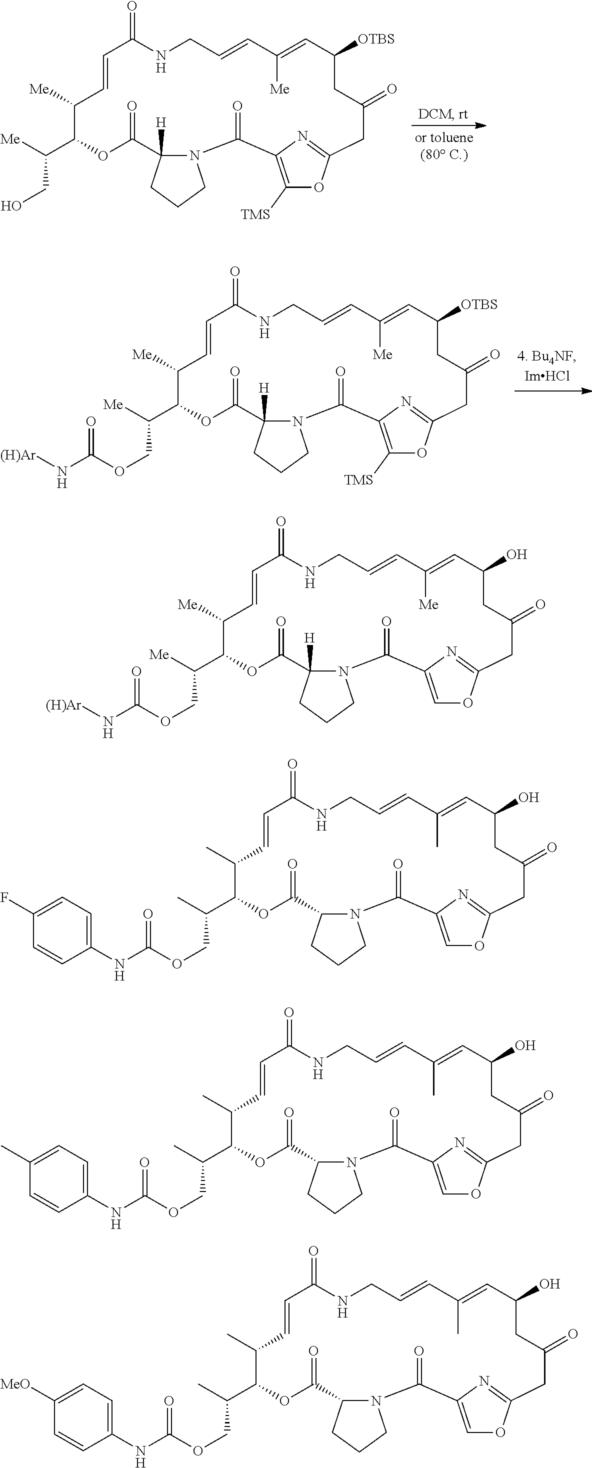

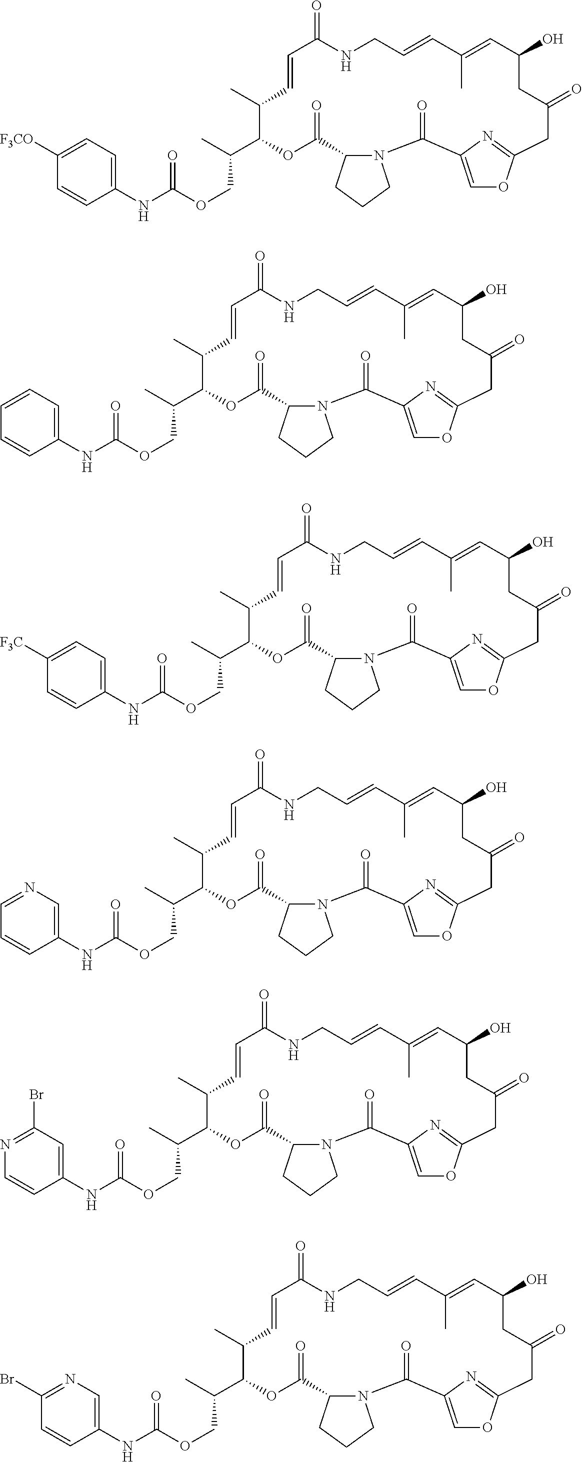

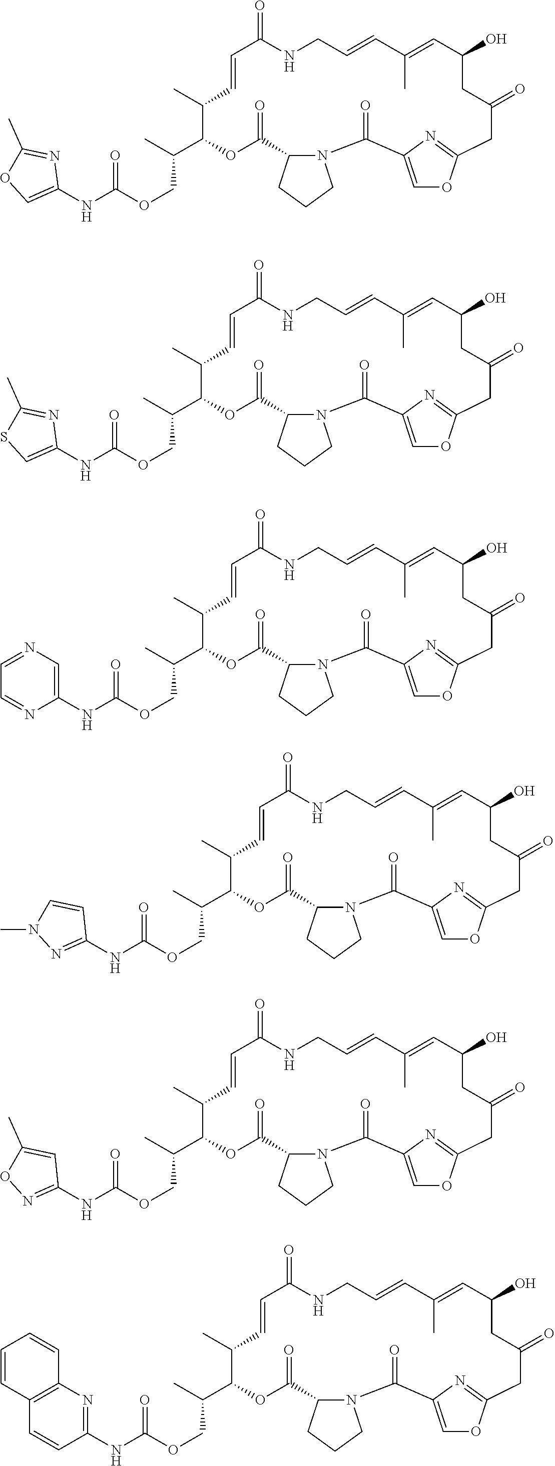

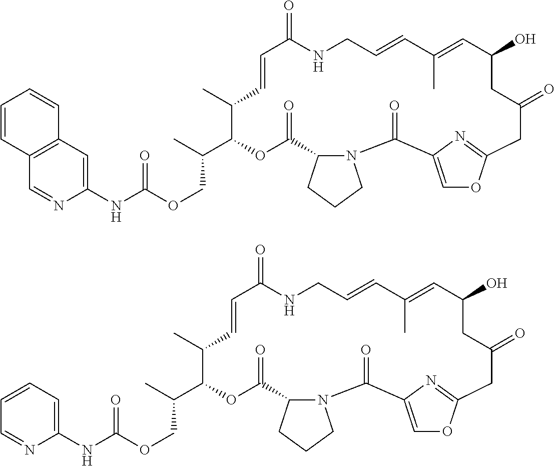 [SMILES: CC1=C\[C@@H](O)CC(=O)Cc2nc(co2)C(=O)N2CCC[C@@H]2C(=O)O[C@H]([C@@H](C)COC(=O)Nc2cc3ccccc3cn2)[C@H](C)/C=C/C(=O)NC\C=C\1 ; CC1=C\[C@@H](O)CC(=O)Cc2nc(co2)C(=O)N2CCC[C@@H]2C(=O)O[C@H]([C@@H](C)COC(=O)Nc2ccccn2)[C@H](C)/C=C/C(=O)NC\C=C\1]

Carbamate Analogues

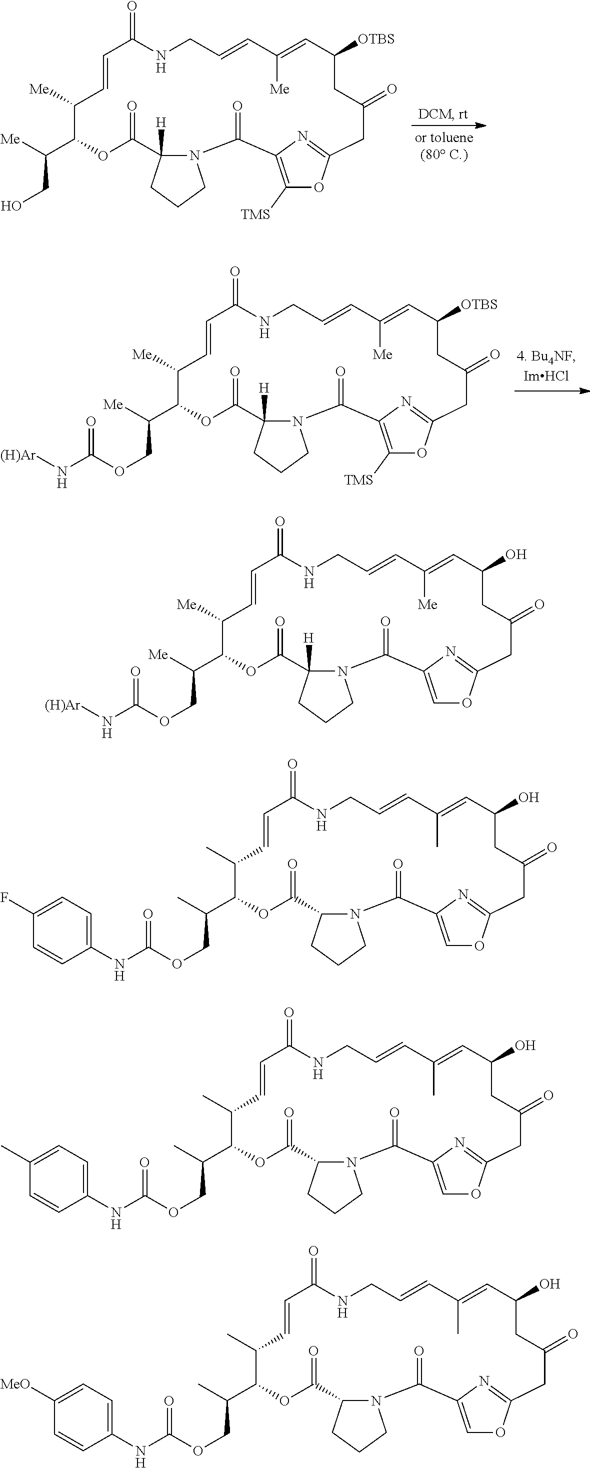

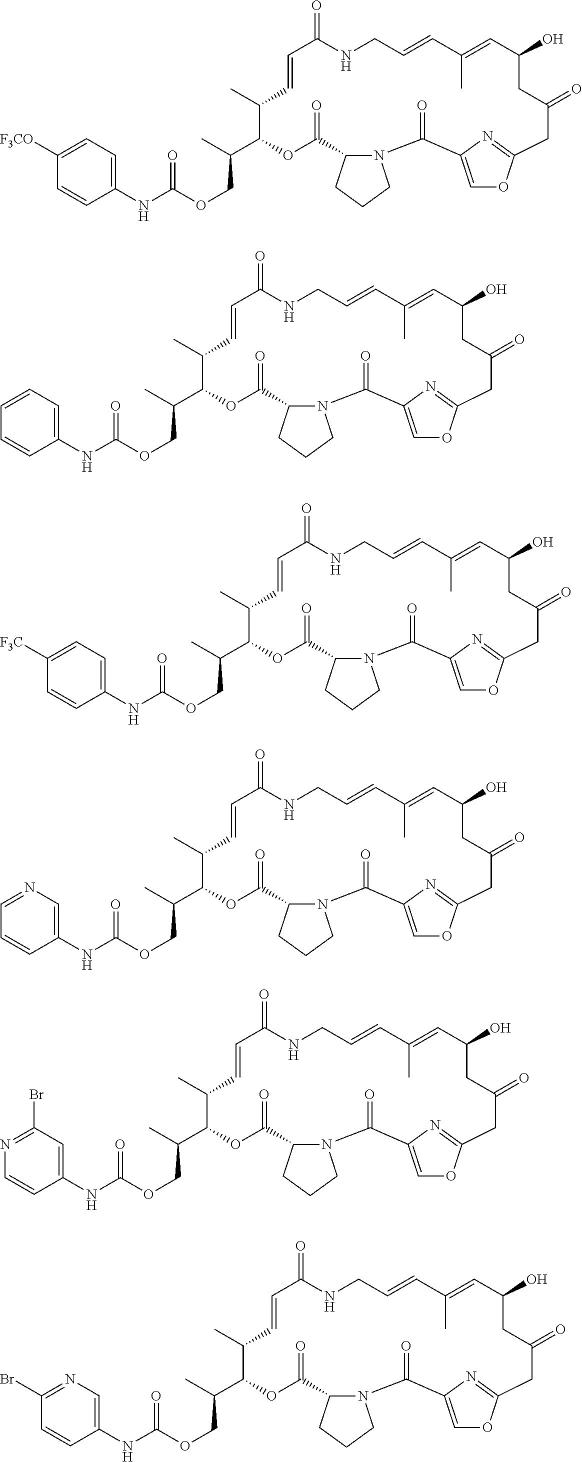

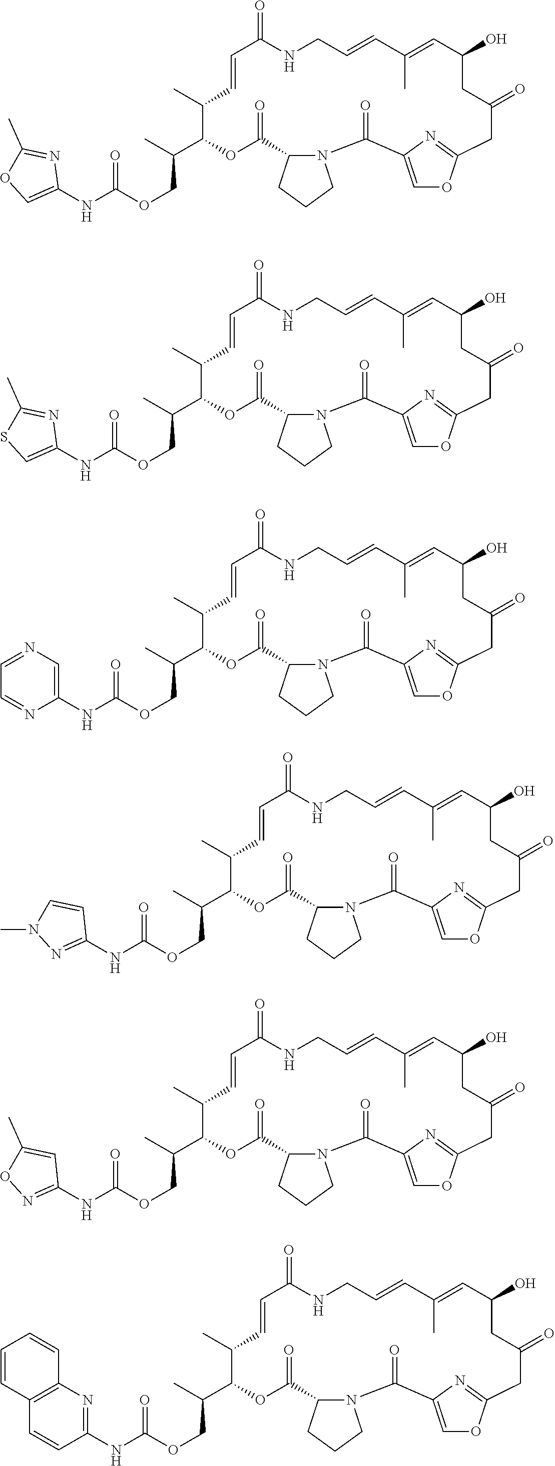

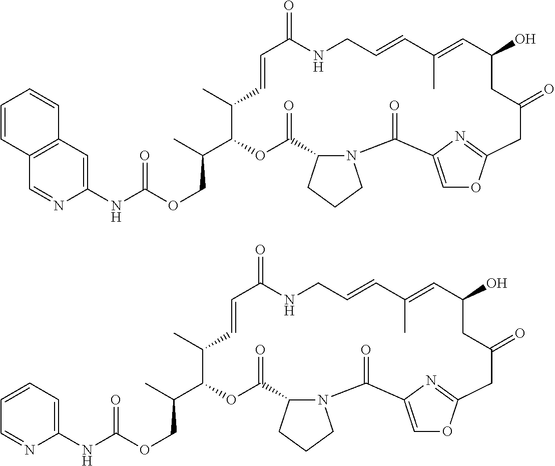 [SMILES: CC1=C\[C@@H](O)CC(=O)Cc2nc(co2)C(=O)N2CCC[C@@H]2C(=O)O[C@H]([C@H](C)COC(=O)Nc2cc3ccccc3cn2)[C@H](C)/C=C/C(=O)NC\C=C\1 ; CC1=C\[C@@H](O)CC(=O)Cc2nc(co2)C(=O)N2CCC[C@@H]2C(=O)O[C@H]([C@H](C)COC(=O)Nc2ccccn2)[C@H](C)/C=C/C(=O)NC\C=C\1]

Additional compounds

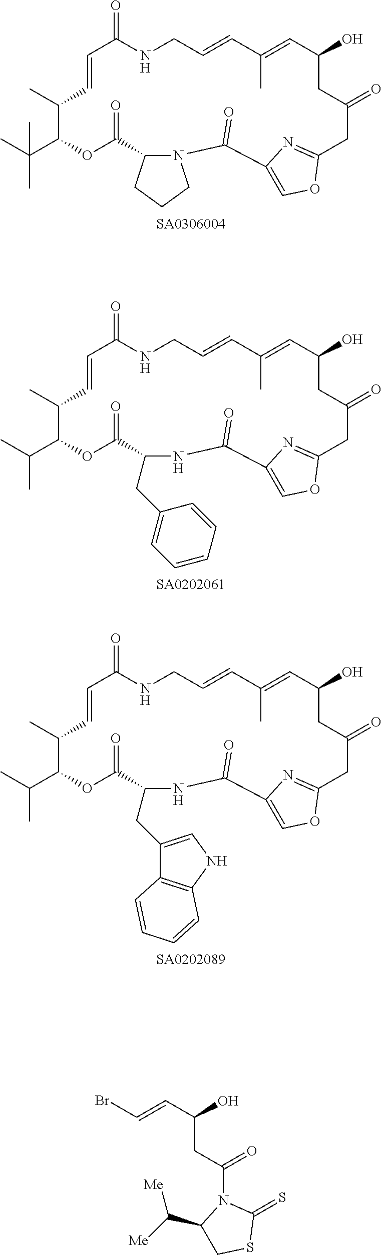 [SMILES: CC1=C\[C@@H](O)CC(=O)Cc2nc(co2)C(=O)N2CCC[C@@H]2C(=O)O[C@H](C(C)(C)C)[C@H](C)/C=C/C(=O)NC\C=C\1 ; CC1=C\[C@@H](O)CC(=O)Cc2nc(co2)C(=O)N[C@H](Cc2ccccc2)C(=O)O[C@H](C(C)C)[C@H](C)/C=C/C(=O)NC\C=C\1 ; CC1=C\[C@@H](O)CC(=O)Cc2nc(co2)C(=O)N[C@H](Cc2c[nH]c3ccccc23)C(=O)O[C@H](C(C)C)[C@H](C)/C=C/C(=O)NC\C=C\1 ; CC(C)[C@@H]1CSC(=S)N1C(=O)C[C@H](O)/C=C/Br]

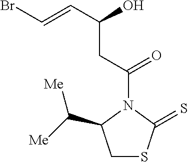 [SMILES: CC(C)[C@@H]1CSC(=S)N1C(=O)C[C@H](O)/C=C/Br]

¹H NMR (400 MHz, Chloroform-d) δ 6.44 (dd, J=13.5, 1.4 Hz, 1H), 6.28 (dd, J=13.6, 5.6 Hz, 1H), 5.15 (ddd, J=7.7, 6.2, 1.1 Hz, 1H), 4.72-4.62 (m, 1H), 3.69 (dd, J=17.7, 3.1 Hz, 1H), 3.54 (dd, J=11.5, 7.9 Hz, 1H), 3.29 (dd, J=17.7, 8.6 Hz, 1H), 3.06 (br s, 1H), 3.04 (dd, J=11.4, 1.1 Hz, 1H), 2.45-2.25 (m, 1H), 1.06 (d, J=6.8 Hz, 3H), 0.98 (d, J=6.9 Hz, 3H).

¹³C NMR (100 MHz, CDCl₃) δ 203.0, 171.8, 137.7, 108.1, 71.3, 68.5, 44.6, 30.8, 30.7, 19.1, 17.8.

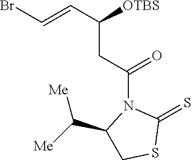 [SMILES: CC(C)[C@@H]1CSC(=S)N1C(=O)C[C@@H](/C=C/Br)O[Si](C)(C)C(C)(C)C]

¹H NMR (400 MHz, Chloroform-d) δ 6.38-6.23 (m, 2H), 5.04 (ddd, J=7.7, 6.3, 1.1 Hz, 1H), 4.73 (ddd, J=7.7, 5.9, 4.6 Hz, 1H), 3.61 (dd, J=16.8, 7.8 Hz, 1H), 3.48 (dd, J=11.5, 7.8 Hz, 1H), 3.26 (dd, J=16.8, 4.6 Hz, 1H), 3.03 (dd, J=11.5, 1.1 Hz, 1H), 2.42-2.28 (m, 1H), 1.06 (d, J=6.8 Hz, 3H), 0.97 (d, J=6.9 Hz, 3H), 0.86 (s, 9H), 0.05 (s, 6H).

¹³C NMR (100 MHz, CDCl₃) δ 202.8, 170.5, 139.5, 107.1, 71.6, 70.0, 45.8, 30.8, 30.7, 25.7, 19.1, 18.0, 17.8, −4.5, −5.0.

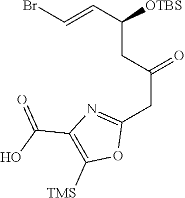 [SMILES: CC(C)(C)[Si](C)(C)O[C@H](/C=C/Br)CC(=O)Cc1nc(C(=O)O)c([Si](C)(C)C)o1]

¹H NMR (400 MHz, Chloroform-d) δ 9.38 (s, 1H), 6.31 (dd, J=13.5, 1.0 Hz, 1H), 6.19 (dd, J=13.6, 6.3 Hz, 1H), 4.64 (dddd, J=7.5, 6.1, 4.9, 1.1 Hz, 1H), 4.15 (d, J=17.0 Hz, 1H), 4.06 (d, J=17.0 Hz, 1H), 2.84 (dd, J=15.9, 7.6 Hz, 1H), 2.64 (dd, J=15.9, 4.9 Hz, 1H), 0.86 (s, 9H), 0.37 (s, 9H), 0.04 (s, 6H).

¹³C NMR (100 MHz, CDCl₃) δ 200.4, 165.3, 165.2, 161.0, 140.8, 139.1, 107.3, 69.5, 49.9, 43.5, 25.7, 18.0, −2.2, −4.6, −5.1.

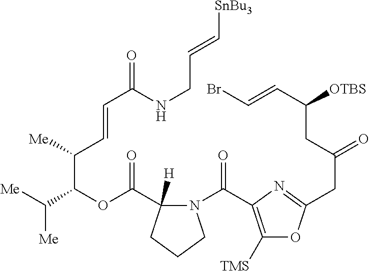

¹H NMR (400 MHz, Chloroform-d) δ 6.76-6.56 (m, 1H), 6.35-6.25 (m, 1H), 6.25-6.16 (m, 1H), 6.11 (dq, J=19.0, 1.5 Hz, 1H), 5.96 (dt, J=19.1, 5.1 Hz, 1H), 5.87-5.72 (m, 1H), 5.72-5.55 (m, 1H), 4.80 (t, J=6.2 Hz, 0.6H), 4.73 (t, J=6.2 Hz, 0.4H), 4.66-4.46 (m, 2H), 4.15-3.64 (m, 6H), 2.88-2.74 (m, 1H), 2.71-2.40 (m, 2H), 2.33-2.15 (m, 1H), 2.10-1.82 (m, 4H), 1.57-1.37 (m, 6H), 1.35-1.32 (m, 6H), 1.07-0.92 (m, 6H), 0.92-0.77 (m, 27H), 0.37-0.27 (m, 9H), 0.07-0.01 (m, 6H). ¹³C NMR (100 MHz, CDCl₃) δ 201.0, 200.5, 172.34, 172.32, 165.4, 165.1, 163.2, 162.5, 161.5, 161.4, 159.01, 158.97, 145.37, 145.26, 145.22, 145.13, 143.4, 143.3, 139.23, 139.20, 130.4, 130.2, 123.9, 123.8, 107.3, 80.8, 80.4, 69.6, 69.5, 60.5, 59.9, 49.8, 48.8, 47.1, 44.9, 44.9, 44.1, 43.8, 38.4, 38.1, 31.6, 29.9, 29.8, 29.1, 29.0, 28.9, 27.2, 25.73, 25.68, 25.2, 21.5, 19.7, 19.5, 18.0, 17.0, 16.9, 14.9, 14.6, 13.7, 9.4, −1.74, −1.77, −1.79, −4.6, −5.10, −5.13.

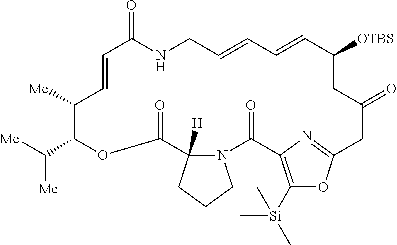 [SMILES: CC(C)[C@H]1OC(=O)[C@H]2CCCN2C(=O)c2nc(oc2[Si](C)(C)C)CC(=O)C[C@H](O[Si](C)(C)C(C)(C)C)/C=C/C=C/CNC(=O)/C=C/[C@H]1C]

¹H NMR (400 MHz, Chloroform-d) δ 6.45 (dd, J=16.3, 4.6 Hz, 1H), 6.19-6.03 (m, 2H), 5.99 (dd, J=7.5, 3.6 Hz, 1H), 5.78 (dd, J=16.3, 1.9 Hz, 1H), 5.72-5.56 (m, 2H), 4.84-4.70 (m, 3H), 4.26 (dt, J=14.3, 6.5 Hz, 1H), 3.90 (d, J=16.4 Hz, 1H), 3.85-3.80 (m, 2H), 3.73 (d, J=16.4 Hz, 1H), 3.56 (ddd, J=15.2, 7.5, 3.5 Hz, 1H), 2.86 (dd, J=16.3, 7.0 Hz, 1H), 2.78-2.68 (m, 1H), 2.70 (dd, J=16.3, 5.7 Hz, 1H), 2.20-2.06 (m, 1H), 2.03-1.77 (m, 4H), 1.08 (d, J=6.8 Hz, 3H), 1.00 (d, J=6.4 Hz, 3H), 0.95 (d, J=6.8 Hz, 3H), 0.87 (s, 9H), 0.30 (s, 9H), 0.07 (s, 3H), 0.04 (s, 3H).

¹³C NMR (100 MHz, CDCl₃) δ 201.0, 172.4, 166.7, 161.7, 161.6, 159.6, 145.2, 144.7, 135.5, 131.9, 129.0, 128.9, 123.8, 81.3, 69.0, 59.1, 50.8, 48.5, 43.3 41.1, 36.7, 29.4, 28.29, 25.8, 25.1, 19.8, 18.6, 18.1, 9.9, −1.8, −4.5, −5.0.

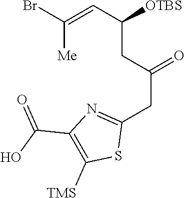 [SMILES: C/C(Br)=C\[C@H](CC(=O)Cc1nc(C(=O)O)c([Si](C)(C)C)s1)O[Si](C)(C)C(C)(C)C]

¹H NMR (400 MHz, Chloroform-d) δ 9.56 (br s, 1H), 5.82 (dd, J=9.1, 1.4 Hz, 1H), 4.82 (ddd, J=9.1, 8.0, 4.8 Hz, 1H), 4.36 (d, J=18.2 Hz, 1H), 4.27 (d, J=18.2 Hz, 1H), 2.89 (dd, J=15.6, 8.0 Hz, 1H), 2.60 (dd, J=15.6, 4.8 Hz, 1H), 2.28 (d, J=1.3 Hz, 3H), 0.83 (s, 9H), 0.40 (s, 9H), 0.04 (s, 3H), 0.03 (s, 3H).

¹³C NMR (100 MHz, CDCl₃) δ 202.2, 165.0, 164.3, 149.6, 146.8, 134.2, 121.8, 66.9, 50.1, 47.4, 25.7, 24.0, 18.0, −0.5, −4.5, −5.1.

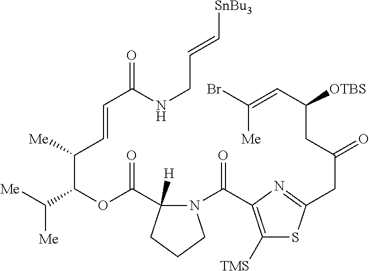

¹H NMR (400 MHz, Chloroform-d) δ 6.74-6.55 (m, 1H), 6.10 (dt, J=19.0, 1.5 Hz, 1H), 5.95 (dtd, J=19.0, 5.0, 2.1 Hz, 1H), 5.89-5.77 (m, 2H), 5.77-5.59 (m, 1H), 4.87-4.75 (m, 2H), 4.75-4.58 (m, 1H), 4.21-3.62 (m, 6H), 2.87 (ddd, J=15.5, 8.0, 2.3 Hz, 1H), 2.73-2.40 (m, 2H), 2.41-2.14 (m, 5H), 2.12-1.87 (m, 3H), 1.60-1.35 (m, 6H), 1.35-1.22 (m, 6H), 1.09-0.92 (m, 6H), 0.92-0.70 (m, 27H), 0.40-0.28 (m, 9H), 0.08-−0.02 (m, 6H).

¹³C NMR (100 MHz, CDCl₃) δ 202.4, 202.2, 172.5, 172.1, 172.0, 165.42, 165.37, 165.0, 163.1, 163.0, 162.9, 162.8, 161.7, 155.0, 154.3, 145.4, 145.2, 145.0, 143.4, 143.3, 142.7, 141.0, 135.2, 134.3, 134.3, 130.3, 130.2, 130.1, 124.0, 123.9, 121.7, 80.7, 80.4, 67.9, 66.94, 66.86, 61.3, 59.8, 49.9, 49.8, 49.2, 48.03, 47.7, 47.4, 44.8, 38.4, 38.1, 31.7, 29.9, 29.8, 29.7, 29.0, 27.2, 25.7, 25.1, 24.00, 23.98, 23.89, 21.9, 19.7, 19.4, 18.0, 17.0, 16.8, 15.1, 14.5, 13.7, 9.4, 0.2, 0.1, −4.6, −5.1.

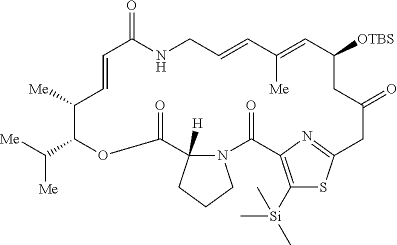 [SMILES: CC1=C\[C@@H](O[Si](C)(C)C(C)(C)C)CC(=O)Cc2nc(c([Si](C)(C)C)s2)C(=O)N2CCC[C@@H]2C(=O)O[C@H](C(C)C)[C@H](C)/C=C/C(=O)NC\C=C\1]

¹H NMR (400 MHz, Chloroform-d) δ 6.49 (dd, J=16.3, 4.1 Hz, 1H), 6.13 (d, J=15.6 Hz, 1H), 6.00 (dd, J=8.8, 2.9 Hz, 1H), 5.77 (dd, J=16.3, 2.0 Hz, 1H), 5.55 (ddd, J=15.5, 9.5, 4.3 Hz, 1H), 5.39 (d, J=9.0 Hz, 1H), 4.97 (ddd, J=9.0, 7.8, 5.3 Hz, 1H), 4.77 (ddd, J=8.7, 6.6, 2.9 Hz, 2H), 4.48 (ddd, J=13.4, 8.9, 4.1 Hz, 1H), 4.10 (d, J=17.2 Hz, 1H), 3.90 (d, J=17.2 Hz, 1H), 3.68-3.50 (m, 2H), 3.39 (ddd, J=14.7, 9.5, 3.3 Hz, 1H), 2.89 (dd, J=16.1, 7.8 Hz, 1H), 2.75 (dd, J=16.0, 5.3 Hz, 1H), 2.79-2.69 (m, 1H), 2.19-2.05 (m, 2H), 1.99-1.89 (m, 1H), 1.89-1.68 (m, 2H), 1.64 (d, J=1.2 Hz, 3H), 1.08 (d, J=6.9 Hz, 3H), 1.00 (d, J=6.4 Hz, 3H), 0.94 (d, J=6.8 Hz, 3H), 0.84 (s, 9H), 0.31 (s, 9H), 0.03 (s, 3H), 0.00 (s, 3H).

¹³C NMR (100 MHz, CDCl₃) δ 202.2, 172.2, 166.4, 163.3, 163.2, 155.4, 144.8, 140.1, 137.0, 134.7, 132.5, 124.7, 123.7, 80.9, 65.3, 58.8, 50.8, 48.8, 47.4, 41.3, 36.6, 29.3, 28.6, 25.7, 25.0, 19.9, 18.6, 18.0, 12.7, 9.7, −0.0, −4.5, −5.0.

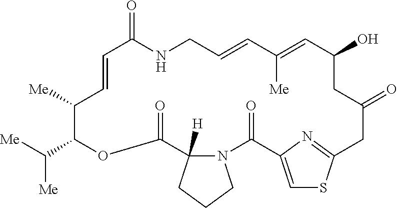 [SMILES: CC1=C\[C@@H](O)CC(=O)Cc2nc(cs2)C(=O)N2CCC[C@@H]2C(=O)O[C@H](C(C)C)[C@H](C)/C=C/C(=O)NC\C=C\1]

¹H NMR (400 MHz, CDCl₃) δ 7.91 (s, 1H), 6.53 (dd, J=16.3, 4.7 Hz, 1H), 6.40 (dd, J=8.4, 4.1 Hz, 1H), 6.12 (d, J=15.6 Hz, 1H), 5.76 (dd, J=16.2, 1.9 Hz, 1H), 5.61 (ddd, J=15.6, 8.9, 4.4 Hz, 1H), 5.40 (d, J=8.9 Hz, 1H), 4.91 (dt, J=8.7, 6.1 Hz, 1H), 4.82-4.64 (m, 2H), 4.32 (ddd, J=13.7, 8.5, 4.6 Hz, 1H), 3.98 (d, J=2.3 Hz, 2H), 3.85 (dt, J=10.9, 7.3 Hz, 1H), 3.71 (ddd, J=11.1, 7.8, 4.9 Hz, 1H), 3.45 (ddd, J=14.8, 9.0, 4.0 Hz, 1H), 3.05 (dd, J=16.8, 6.6 Hz, 2H), 2.82 (dd, J=16.8, 5.7 Hz, 1H), 2.73 (ddq, J=6.9, 4.4, 2.3 Hz, 1H), 2.14 (dtd, J=13.2, 9.4, 7.4 Hz, 1H), 1.99-1.71 (m, 4H), 1.68 (d, J=1.2 Hz, 3H), 1.05 (d, J=6.8 Hz, 3H), 0.98 (d, J=6.5 Hz, 3H), 0.94 (d, J=6.7 Hz, 3H).

¹³C NMR (100 MHz, CDCl₃) δ 203.5, 171.7, 166.4, 161.4, 160.6, 150.6, 144.9, 136.5, 134.4, 132.7, 126.1, 125.2, 123.9, 81.2, 64.7, 59.6, 49.4, 49.1, 47.5, 40.9, 36.57, 29.4, 28.5, 25.1, 19.7, 18.6, 12.7, 10.2.

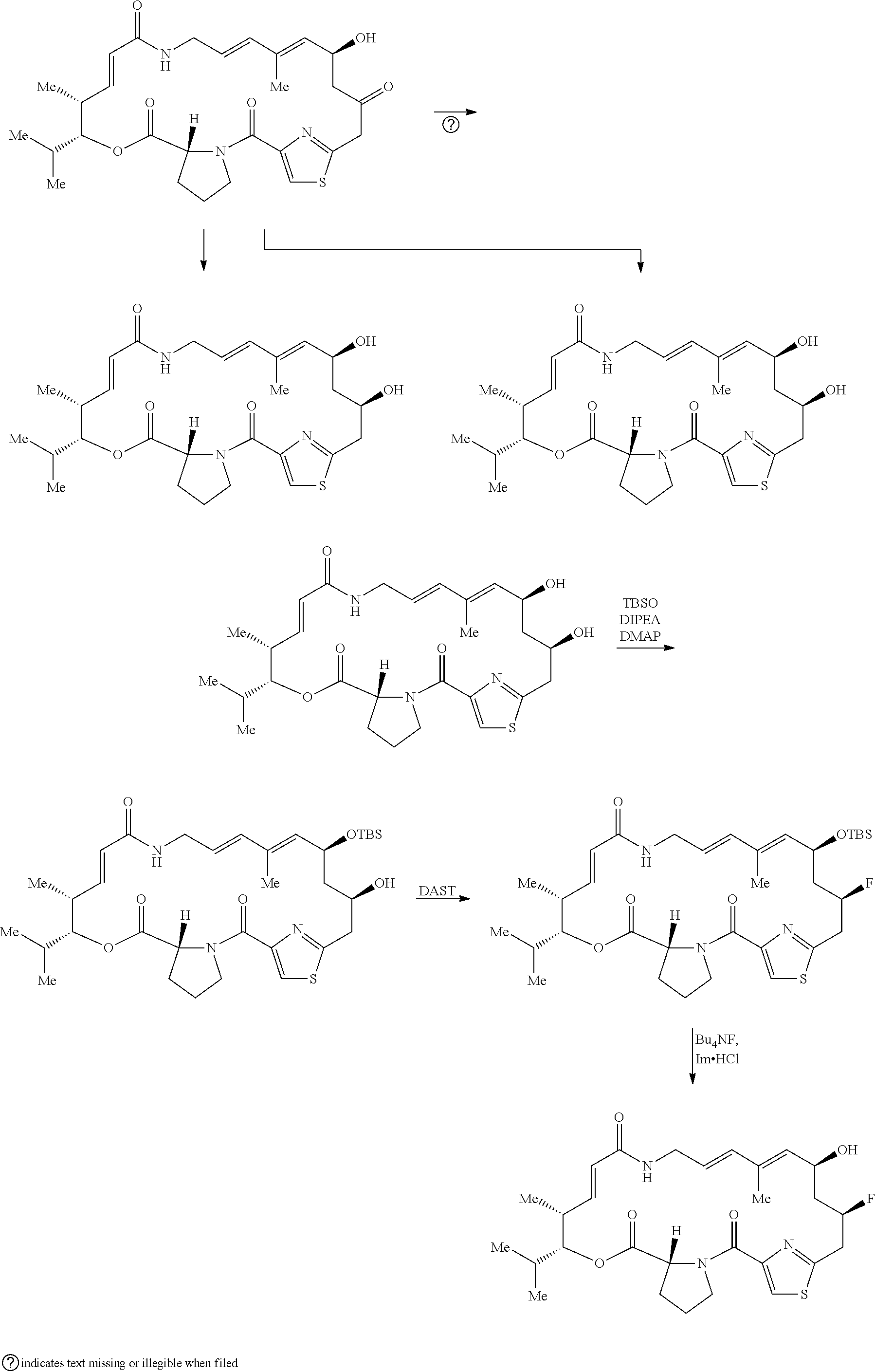

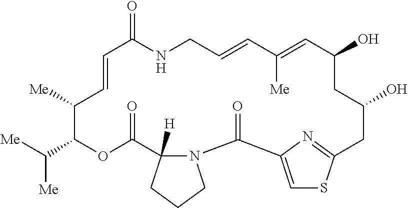 [SMILES: CC1=C\[C@@H](O)C[C@H](O)Cc2nc(cs2)C(=O)N2CCC[C@@H]2C(=O)O[C@H](C(C)C)[C@H](C)/C=C/C(=O)NC\C=C\1]

An oven-dried 50-mL round-bottom flask charged with tetramethylammonium triacetoxyhydroborate (0.12 g, 5.000 Eq, 0.46 mmol) was evacuated and flushed with nitrogen (this process was repeated a total of 3 times) and was sealed with a rubber septum. Acetonitrile (5 mL) and acetic acid (1 mL) was added, and the resulting colorless solution was cooled to −10° C. by means of ice-acetone bath. A solution of (12Z,32R,6R,7R,8E,13E,15E,17S)-17-hydroxy-6-isopropyl-7,15-dimethyl-5-oxa-11-aza-1(4,2)-thiazola-3(1,2)-pyrrolidinacycloicosaphane-8,13,15-triene-2,4,10,19-tetraone (0.050 g, 1 Eq, 92 μmol) in acetonitrile (5 mL) was added dropwise. The mixture was allowed to warm 15 to 23° C. slowly. After stirring for 5 hours, aqueous sat. NaHCO₃ solution was added (CAUTION: Gas evolution). EtOAc (30 mL) was added and the biphasic mixture was transferred to a separatory funnel and the layers were separated. The aqueous layer was extracted with EtOAc (2×10 mL). The combined organic layers were washed with water (50 mL) and brine (50 mL) and the washed solution was dried (Na₂SO₄). The dried solution was filtered and the filtrate was concentrated. The crude residue was purified by flash chromatography (silica gel, eluent: MeOH:DCM=1:25) to afford (12Z,32R,6R,7R,8E,13E,15E,17S,19S)-17,19-dihydroxy-6-isopropyl-7,15-dimethyl-5-oxa-11-aza-1(4,2)-thiazola-3(1,2)-pyrrolidinacycloicosaphane-8,13,15-triene-2,4,10-trione (0.045 g, 90%) as a white solid.

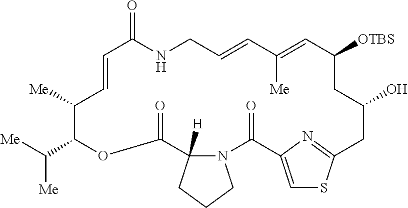 [SMILES: CC1=C\[C@@H](O[Si](C)(C)C(C)(C)C)C[C@H](O)Cc2nc(cs2)C(=O)N2CCC[C@@H]2C(=O)O[C@H](C(C)C)[C@H](C)/C=C/C(=O)NC\C=C\1]

To a solution of (12Z,32R,6R,7R,8E,13E,15E,17S,19S)-17,19-dihydroxy-6-isopropyl-7,15-dimethyl-5-oxa-11-aza-1(4,2)-thiazola-3(1,2)-pyrrolidinacycloicosaphane-8,13,15-triene-2,4,10-trione (0.045 g, 1 Eq, 82 μmol) and DMAP (1.0 mg, 0.100 Eq, 8.2 μmol) in DCM (8 mL) was added DIPEA (0.16 g, 0.22 mL, 15.000 Eq, 1.2 mmol) and TBS-C₁ (0.19 g, 15.000 Eq, 1.2 mmol) at rt. After stirring for 24 hours. The reaction was diluted with DCM and transferred to a separate funnel. The organic solution was washed with water and brine. The washed solution was dried with Na2SO4 and the dried solution was concentrated under vacuum. The residue was purified by flash chromatography (Acetone:Hexanes=1:5 to 1:2) to afford (12Z,32R,6R,7R,8E,13E,15E,17S,19S)-17-((tert-butyldimethylsilyl)oxy)-19-hydroxy-6-isopropyl-7,15-dimethyl-5-oxa-11-aza-1(4,2)-thiazola-3(1,2)-pyrrolidinacycloicosaphane-8,13,15-triene-2,4,10-trione (0.043 g, 79%) as a white solid.

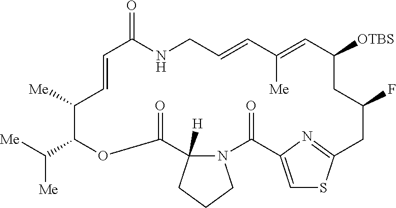 [SMILES: CC1=C\[C@@H](O[Si](C)(C)C(C)(C)C)C[C@@H](F)Cc2nc(cs2)C(=O)N2CCC[C@@H]2C(=O)O[C@H](C(C)C)[C@H](C)/C=C/C(=O)NC\C=C\1]

To a solution of (12Z,32R,6R,7R,8E,13E,15E,17S,19S)-17-((tert-butyldimethylsilyl)oxy)-19-hydroxy-6-isopropyl-7,15-dimethyl-5-oxa-11-aza-1(4,2)-thiazola-3(1,2)-pyrrolidinacycloicosaphane-8,13,15-triene-2,4,10-trione (0.042 g, 1 Eq, 64 μmol) in DCM (5 mL) was added DAST (15 mg, 13 μL, 1.500 Eq, 95 μmol) drop wise at 0° C. under N₂. The reaction was warmed to RT and stirred for 3 hours. The reaction mixture was quenched with aqueous sat. NaHCO₃ solution, diluted with 20 mL DCM and transferred to a separate funnel. The organic solution was washed with water and brine. The washed solution was dried with Na₂SO₄ and the dried solution was concentrated under vacuum. The residue was purified by flash chromatography (Acetone:Hexanes=1:5 to 1:2) to afford (12Z,32R,6R,7R,8E,13E,15E,17S,19R)-17-((tert-butyldimethylsilyl)oxy)-19-fluoro-6-isopropyl-7,15-dimethyl-5-oxa-11-aza-1(4,2)-thiazola-3(1,2)-pyrrolidinacycloicosaphane-8,13,15-triene-2,4,10-trione (0.0400 g, 95%) as a white solid.

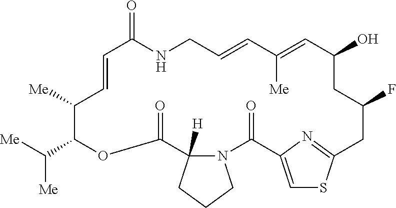 [SMILES: CC1=C\[C@@H](O)C[C@@H](F)Cc2nc(cs2)C(=O)N2CCC[C@@H]2C(=O)O[C@H](C(C)C)[C@H](C)/C=C/C(=O)NC\C=C\1]

An oven-dried 100-mL round-bottom flask charged with (12Z,32R,6R,7R,8E,13E,15E,17S,19R)-17-((tert-butyldimethylsilyl)oxy)-19-fluoro-6-isopropyl-7,15-dimethyl-5-oxa-11-aza-1(4,2)-thiazola-3(1,2)-pyrrolidinacycloicosaphane-8,13,15-triene-2,4,10-trione (0.020 g, 1 Eq, 30 μmol)) was evacuated and filled with nitrogen (this process was repeated a total of 3 times) and was sealed with a rubber septum. THF (3 mL) was added, resulting in a light yellow solution. In a separate flask, 1H-imidazol-3-ium chloride (47 mg, 0.45 mL, 15.000 Eq, 0.45 mmol) was added to a solution of tetrabutylammonium fluoride (0.12 g, 0.45 mL, 15.000 Eq, 0.45 mmol) in THF The resulting colorless solution was added dropwise to the solution. After 12 h, the mixture was concentrated and the residue was dissolved in DCM (50 mL). The resulting solution was transferred to a separatory funnel and was washed with water (5×50 mL) and brine (50 mL). The washed solution was dried Na₂SO₄). The dried solution was filtered and the filtrate was concentrated. The resulting crude residue was purified by flashed chromatography (silica gel, eluent: MeOH:DCM=1:100 to 1:50) to afford (12Z,32R,6R,7R,8E,13E,15E,17S,19R)-19-fluoro-17-hydroxy-6-isopropyl-7,15-dimethyl-5-oxa-11-aza-1(4,2)-thiazola-3(1,2)-pyrrolidinacycloicosaphane-8,13,15-triene-2,4,10-trione (0.016 g, 97%) as a white solid.

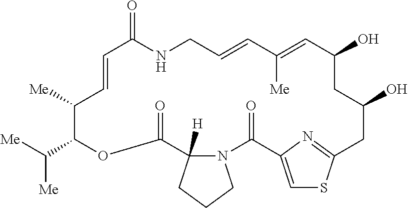 [SMILES: CC1=C\[C@@H](O)C[C@@H](O)Cc2nc(cs2)C(=O)N2CCC[C@@H]2C(=O)O[C@H](C(C)C)[C@H](C)/C=C/C(=O)NC\C=C\1]

The SM was dried over azeotrope with Toluene twice. Then to a solution of (12Z,32R,6R,7R,8E,13E,15E,17S)-17-hydroxy-6-isopropyl-7,15-dimethyl-5-oxa-11-aza-1(4,2)-thiazola-3(1,2)-pyrrolidinacycloicosaphane-8,13,15-triene-2,4,10,19-tetraone (0.027 g, 1 Eq, 50 μmol) in dry THF (2 mL) and dry MeOH (5 mL) was added diethyl(methoxy)borane (6.0 mg, 60 μL, 1.200 Eq, 60 μmol) at −78° C. under N₂. After stirring for 30 min, sodium borohydride (2.8 mg, 1.500 Eq, 74 μmol) was added. After stirring for 3 hours, the reaction mixture was quenched with acetic acid (0.25 mL), diluted with EtOAc and warmed to RT. The organic mixture was washed with water and brine, dried over Na₂SO₄ and concentrated under vacuum. The resulting residue was purified by flashed chromatography (silica gel, MeOH:DCM=1:50 to 1:20) to afford (12Z,32R,6R,7R,8E,13E,15E,17S,19R)-17,19-dihydroxy-6-isopropyl-7,15-dimethyl-5-oxa-11-aza-1(4,2)-thiazola-3(1,2)-pyrrolidinacycloicosaphane-8,13,15-triene-2,4,10-trione (0.021 g, 77%)

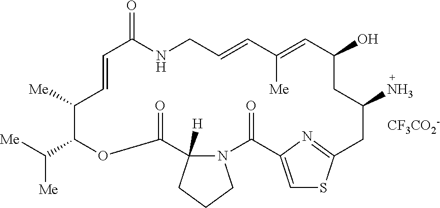

To a solution of (12Z,32R,6R,7R,8E,13E,15E,17S)-17-hydroxy-6-isopropyl-7,15-dimethyl-5-oxa-11-aza-1(4,2)-thiazola-3(1,2)-pyrrolidinacycloicosaphane-8,13,15-triene-2,4,10,19-tetraone (0.027 g, 1 Eq, 50 μmol) in methanol (5 mL) was added magnesium sulfate (60 mg, 10.00 Eq, 0.50 mmol), ammonium acetate (19 mg, 5.000 Eq, 0.25 mmol) and sodium cyanoborohydride (7.5 mg, 2.400 Eq, 0.12 mmol) at 20° C., under an argon atmosphere, After stirring for 20 hours, the reaction mixture is filtered on Celite, and then the Celite rinsed with methanol. The filtrate is concentrated underreduced pressure to give a chestnut-coloured oil. The residue was purified by prepared HPLC to afford (12Z,32R,6R,7R,8E,13E,15E,17S,19R)-17-hydroxy-6-isopropyl-7,15-dimethyl-2,4,10-trioxo-5-oxa-11-aza-1(4,2)-thiazola-3(1,2)-pyrrolidinacycloicosaphane-8,13,15-trien-19-aminium 2,2,2-trifluoroacetate (0.010 g, 31%) as a white solid.

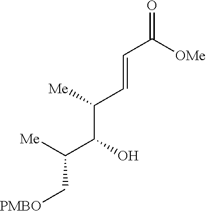

¹H NMR (400 MHz, Chloroform-d) δ 7.23 (d, J=8.7 Hz, 2H), 6.87 (d, J=8.7 Hz, 2H), 6.79 (dd, J=15.7, 9.3 Hz, 1H), 5.85 (dd, J=15.7, 0.7 Hz, 1H), 4.45 (d, J=11.6 Hz, 1H), 4.41 (d, J=11.6 Hz, 1H), 3.80 (s, 3H), 3.72 (s, 3H), 3.63 (dt, J=9.0, 2.3 Hz, 1H), 3.52 (dd, J=9.0, 3.9 Hz, 1H), 3.46 (dd, J=9.0, 4.9 Hz, 1H), 2.85 (d, J=2.9 Hz, 1H), 2.51-2.38 (m, 1H), 1.78 (dddd, J=7.9, 7.0, 6.1, 2.6 Hz, 1H), 1.14 (d, J=6.6 Hz, 3H), 0.94 (d, J=7.0 Hz, 3H).

¹³C NMR (100 MHz, CDCl₃) δ 167.0, 159.2, 151.1, 129.9, 129.2, 120.8, 113.8, 76.9, 75.2, 73.2, 55.3, 51.5, 40.8, 35.8, 16.7, 9.7.

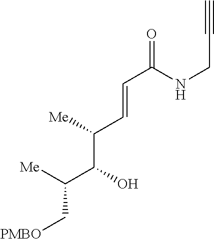

¹H NMR (400 MHz, Chloroform-d) δ 7.21 (d, J=8.7 Hz, 2H), 6.86 (d, J=8.7 Hz, 2H), 6.70 (dd, J=15.3, 9.2 Hz, 1H), 5.92 (brt, J=5.2 Hz, 1H), 5.79 (dd, J=15.3, 0.9 Hz, 1H), 4.43 (d, J=11.5 Hz, 1H), 4.39 (d, J=11.5 Hz, 1H), 4.09 (dd, J=5.3, 2.6 Hz, 2H), 3.79 (s, 3H), 3.61 (dt, J=9.0, 2.2 Hz, 1H), 3.51 (dd, J=9.0, 3.9 Hz, 1H), 3.45 (dd, J=9.0, 4.8 Hz, 1H), 2.95 (d, J=2.8 Hz, 1H), 2.47-2.34 (m, 1H), 2.23 (t, J=2.6 Hz, 1H), 1.84-1.71 (m, 1H), 1.12 (d, J=6.6 Hz, 3H), 0.92 (d, J=7.0 Hz, 3H).

¹³C NMR (100 MHz, CDCl₃) δ 165.3, 159.2, 147.4, 129.9, 129.2, 122.6, 113.8, 79.4, 76.94, 76.7, 75.2, 73.1, 71.6, 55.2, 40.5, 35.7, 29.1, 16.8, 9.8.

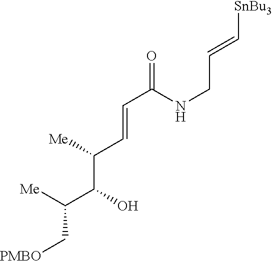

¹H NMR (400 MHz, Chloroform-d) δ 7.22 (d, J=8.6 Hz, 2H), 6.87 (d, J=8.6 Hz, 2H), 6.69 (dd, J=15.2, 9.2 Hz, 1H), 6.12 (dt, J=19.0, 1.5 Hz, 1H), 5.97 (dt, J=19.0, 5.1 Hz, 1H), 5.81 (dd, J=15.3, 0.8 Hz, 1H), 5.50 (br t, J=5.9 Hz, 1H), 4.45 (d, J=11.6 Hz, 1H), 4.40 (d, J=11.6 Hz, 1H), 4.04-3.92 (m, 2H), 3.80 (s, 3H), 3.63 (dt, J=9.0, 2.4 Hz, 1H), 3.53 (dd, J=9.0, 3.8 Hz, 1H), 3.46 (dd, J=9.0, 4.6 Hz, 1H), 2.91 (d, J=2.6 Hz, 1H), 2.50-2.35 (m, 1H), 1.88-1.76 (m, 1H), 1.54-1.41 (m, 6H), 1.30 (h, J=7.3 Hz, 6H), 1.14 (d, J=6.6 Hz, 3H), 0.95 (d, J=7.1 Hz, 3H), 0.88 (t, J=7.3 Hz, 15H).

¹³C NMR (100 MHz, CDCl₃) δ 165.3, 159.2, 146.5, 143.4, 130.5, 130.0, 129.2, 123.4, 113.8, 77.2, 75.4, 73.2, 55.3, 44.9, 40.6, 35.66, 29.0, 27.2, 16.9, 13.7, 9.8, 9.4.

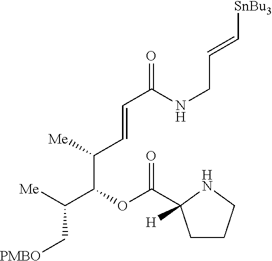

¹H NMR (400 MHz, Chloroform-d) δ 7.23 (d, J=8.6 Hz, 2H), 6.86 (d, J=8.7 Hz, 2H), 6.68 (dd, J=15.4, 8.2 Hz, 1H), 6.11 (dt, J=19.0, 1.5 Hz, 1H), 5.95 (dt, J=18.9, 5.1 Hz, 1H), 5.81 (dd, J=15.5, 1.1 Hz, 1H), 5.55 (brs, 1H), 5.11 (dd, J=8.1, 3.6 Hz, 1H), 4.38 (d, J=11.5 Hz, 1H), 4.34 (d, J=11.5 Hz, 1H), 4.00-3.90 (m, 2H), 3.79 (s, 3H), 3.72 (dd, J=8.5, 5.6 Hz, 1H), 3.34-3.17 (m, 2H), 3.12-2.99 (m, 1H), 2.88 (ddd, J=10.2, 7.1, 6.2 Hz, 1H), 2.73-2.59 (m, 1H), 2.18-1.95 (m, 4H), 1.93-1.75 (m, 1H), 1.75-1.61 (m, 1H), 1.57-1.38 (m, 6H), 1.38-1.24 (m, 6H), 1.03 (d, J=6.7 Hz, 3H), 0.96-0.81 (m, 18H).

¹³C NMR (100 MHz, CDCl₃) δ 175.0, 165.1, 159.1, 144.7, 143.4, 130.4, 130.3, 129.3, 124.1, 113.7, 76.5, 72.9, 72.5, 59.9, 55.2, 46.9, 44.9, 38.5, 35.5, 30.4, 29.0, 27.2, 25.4, 15.8, 13.7, 11.2, 9.4.

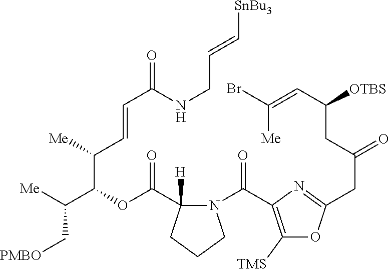

¹H NMR (400 MHz, Chloroform-d) δ 7.26-7.15 (m, 2H), 6.91-6.78 (m, 2H), 6.63 (m, 1H), 6.10 (m, 1H), 6.01-5.91 (m, 1H), 5.85-5.68 (m, 2H), 5.68-5.53 (m, 1H), 5.05 (m, 1H), 4.82-4.71 (m, 1H), 4.64 (m, 1H), 4.45-4.22 (m, 2H), 4.11-3.64 (m, 9H), 3.33 (m, 1H), 3.17 (d, J=6.6 Hz, 1H), 2.81 (m, 1H), 2.69-2.42 (m, 2H), 2.26 (m, 3H), 2.22-1.84 (m, 5H), 1.47 (m, 6H), 1.29 (m, 6H), 1.08-0.73 (m, 30H), 0.39-0.25 (m, 9H), 0.08-0.00 (m, 6H).

¹³C NMR (100 MHz, CDCl₃) δ 201.1, 200.6, 172.1, 171.9, 165.3, 165.1, 163.4, 162.3, 161.5, 161.4, 159.2, 159.1, 159.04, 159.00, 145.2, 145.1, 144.8, 144.6, 143.43, 143.35, 134.2, 130.7, 130.31, 130.3, 130.2, 129.3, 129.2, 124.19, 124.15, 121.8, 113.70, 113.65, 72.8, 72.7, 72.6, 72.3, 67.0, 66.8, 60.6, 59.8, 55.2, 49.6, 48.8, 47.1, 44.9, 44.2, 43.9, 38.7, 38.5, 35.8, 35.4, 31.6, 29.0, 27.2, 25.7, 25.7, 25.6, 25.2, 24.0, 21.5, 18.0, 15.7, 13.7, 11.4, 11.3, 9.4, −1.73, −1.77, −4.6, −5.14, −5.16.

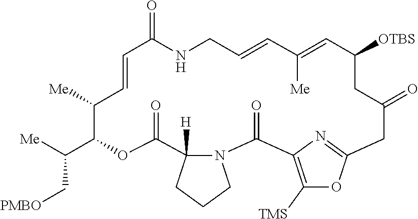

¹H NMR (400 MHz, Chloroform-d) δ 7.21 (d, J=8.7 Hz, 2H), 6.87 (d, J=8.7 Hz, 2H), 6.47 (dd, J=16.3, 4.1 Hz, 1H), 6.20-6.10 (m, 2H), 5.75 (dd, J=16.3, 2.1 Hz, 1H), 5.56 (ddd, J=15.5, 9.5, 4.2 Hz, 1H), 5.41 (d, J=8.9 Hz, 1H), 5.08 (dd, J=9.6, 1.8 Hz, 1H), 5.00 (ddd, J=8.9, 7.1, 5.8 Hz, 1H), 4.80 (dd, J=8.6, 3.3 Hz, 1H), 4.51 (ddd, J=13.9, 9.0, 4.1 Hz, 1H), 4.43 (d, J=11.7 Hz, 1H), 4.38 (d, J=11.7 Hz, 1H), 3.89 (d, J=17.2 Hz, 1H), 3.80 (s, 3H), 3.74 (d, J=17.2 Hz, 1H), 3.74-3.68 (m, 2H), 3.37 (qt, J=9.4, 4.1 Hz, 3H), 2.91 (dd, J=15.7, 7.2 Hz, 1H), 2.73 (dd, J=15.7, 7.2 Hz, 1H), 2.80-2.69 (m, 1H), 2.17-2.03 (m, 2H), 1.91-1.78 (m, 2H), 1.78-1.67 (m, 1H), 1.65 (d, J=1.2 Hz, 3H), 1.08 (d, J=6.9 Hz, 3H), 1.04 (s, 3H), 0.85 (s, 9H), 0.30 (s, 9H), 0.05 (s, 3H), 0.02 (s, 3H).

¹³C NMR (100 MHz, CDCl₃) δ 201.1, 172.0, 166.5, 161.8, 161.3, 159.6, 159.2, 145.0, 144.8, 136.6, 134.6, 132.5, 130.2, 129.1, 125.0, 123.5, 113.8, 78.1, 72.9, 71.9, 65.4, 58.7, 55.3, 50.6, 48.4, 43.8, 41.3, 37.2, 35.1, 28.3, 25.7, 24.8, 18.1, 14.9, 12.7, 10.6, −1.8, −4.5, −5.0.

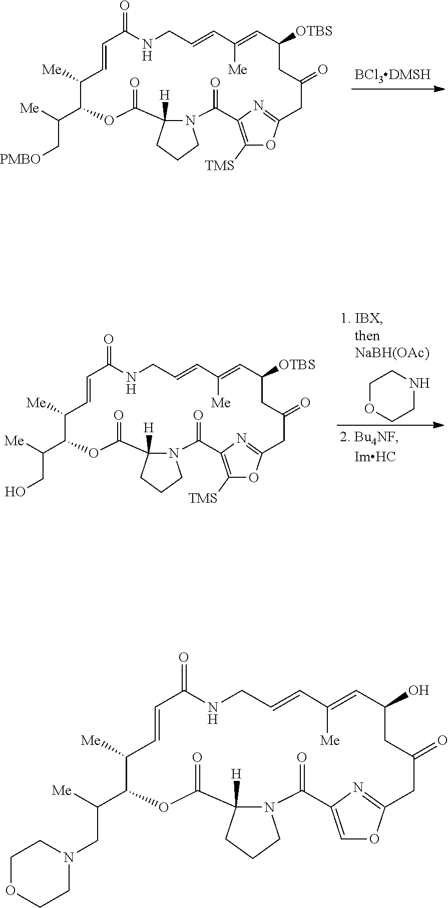

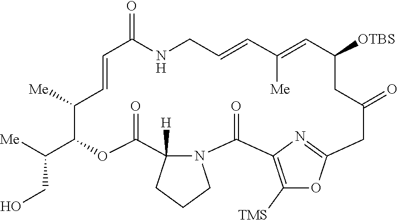 [SMILES: CC1=C\[C@@H](O[Si](C)(C)C(C)(C)C)CC(=O)Cc2nc(c([Si](C)(C)C)o2)C(=O)N2CCC[C@@H]2C(=O)O[C@H]([C@@H](C)CO)[C@H](C)/C=C/C(=O)NC\C=C\1]

A oven-fried flask charged with (12Z,6S,7R,8E,13E,15E)-17-((tert-butyldimethylsilyl)oxy)-6-((S)-1-((4-methoxybenzyl)oxy)propan-2-yl)-7,15-dimethyl-15-(trimethylsilyl)-5-oxa-11-aza-1(4,2)-oxazola-3(1,2)-pyrrolidinacycloicosaphane-8,13,15-triene-2,4,10,19-tetraone (0.830 g, 1 Eq, 976 μmol) was evacuated and refilled with N₂ (this process was repeated 3 times). DCM (98 mL) was added, resulting a yellow solution. The reaction mixture was cooled down to 0° C. and Boron trichloride methyl sulfide complex (280 mg, 781 μL, 1.600 Eq, 1.56 mmol) was added dropwise. After stirring for another 20 min, saturated NaHCO₃ (20 mL) was added. The two phases mixture was stirred for 1 hour at 0° C. and transferred to a separatory funnel. The organic layer was washed with water (2×100 mL) and brine (100 mL). The washed solution was dried (Na₂SO₄). The dried solution was filtered and the filtrate was concentrated. The resulting crude residue was purified by flash chromatography (silica gel, eluent: Acetone:hexanes=1:3.5 to 1:2.5) to afford (12Z,32R,6S,7R,8E,13E,15E,17S)-17-((tert-butyldimethylsilyl)oxy)-6-((S)-1-hydroxypropan-2-yl)-7,15-dimethyl-15-(trimethylsilyl)-5-oxa-11-aza-1(4,2)-oxazola-3(1,2)-pyrrolidinacycloicosaphane-8,13,15-triene-2,4,10,19-tetraone (0.40 g, 56%) as a light yellow solid.

¹H NMR (400 MHz, Chloroform-d) δ 6.51 (dd, J=16.3, 4.3 Hz, 1H), 6.19 (dd, J=8.9, 3.2 Hz, 1H), 6.14 (d, J=15.7 Hz, 1H), 5.77 (dd, J=16.3, 2.0 Hz, 1H), 5.56 (ddd, J=15.5, 9.3, 4.2 Hz, 1H), 5.41 (d, J=8.9 Hz, 1H), 5.16 (dd, J=8.6, 1.9 Hz, 1H), 4.99 (ddd, J=8.9, 7.2, 5.8 Hz, 1H), 4.79 (dd, J=8.6, 3.5 Hz, 1H), 4.49 (ddd, J=13.6, 8.8, 4.2 Hz, 1H), 3.89 (d, J=17.2 Hz, 1H), 3.74 (d, J=17.2 Hz, 1H), 3.82-3.67 (m, 3H), 3.59 (d, J=5.0 Hz, 2H), 3.39 (ddd, J=14.8, 9.4, 3.2 Hz, 1H), 2.91 (dd, J=15.7, 7.2 Hz, 1H), 2.83 (ddq, J=6.6, 4.3, 2.3 Hz, 1H), 2.74 (dd, J=15.7, 5.8 Hz, 1H), 2.20-2.06 (m, 2H), 2.06-1.96 (m, 1H), 1.94-1.80 (m, 2H), 1.65 (d, J=1.2 Hz, 3H), 1.13 (d, J=6.9 Hz, 3H), 1.03 (d, J=6.7 Hz, 3H), 0.85 (s, 9H), 0.30 (s, 9H), 0.05 (s, 3H), 0.01 (s, 3H).

¹³C NMR (100 MHz, CDCl₃) δ 201.1, 172.1, 166.4, 161.9, 161.3, 159.7, 144.94, 144.85, 136.6, 134.6, 132.5, 124.9, 123.6, 77.0, 65.4, 64.6, 58.8, 50.6, 48.5, 43.7, 41.3, 37.4, 37.0, 28.3, 25.7, 24.9, 18.1, 13.9, 12.7, 11.0, −1.8, −4.51, −4.97.

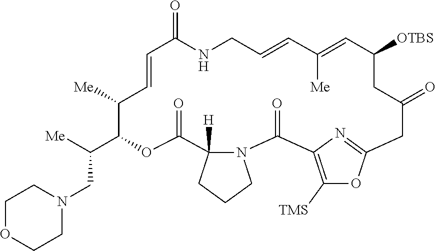 [SMILES: CC1=C\[C@@H](O[Si](C)(C)C(C)(C)C)CC(=O)Cc2nc(c([Si](C)(C)C)o2)C(=O)N2CCC[C@@H]2C(=O)O[C@H]([C@@H](C)CN2CCOCC2)[C@H](C)/C=C/C(=O)NC\C=C\1]

To a solution of (12Z,32R,6S,7R,8E,13E,15E,17S)-17-((tert-butyldimethylsilyl)oxy)-6-((R)-1-hydroxypropan-2-yl)-7,15-dimethyl-15-(trimethylsilyl)-5-oxa-11-aza-1(4,2)-oxazola-3(1,2)-pyrrolidinacycloicosaphane-8,13,15-triene-2,4,10,19-tetraone (0.0300 g, 1 Eq, 41.1 μmol) in EtOAc was added 1-hydroxy-1-oxo-115-benzo[d][1,2]iodaoxol-3(1H)-one (34.5 mg, 3.000 Eq, 123 μmol) and the resulting suspension was heated at 80° C. for 3 hours. The reaction was cooled to room temperature and filtered through a pad of celite. The filter cake was washed with 3×2 mL of ethyl acetate, and the combined filtrates were concentrated to yield a crude aldehyde which was used without further purification.

An oven-dried 25-mL round-bottom flask charged with sodium triacetoxyhydroborate (17.4 mg, 2.000 Eq, 82.2 μmol) was evacuated and filled with nitrogen (this process was repeated a total of 3 times) and was sealed with a rubber septum. DCE (2 mL) and morpholine (7.16 mg, 7.16 μL, 2.000 Eq, 82.2 μmol) was added, resulting in a light yellow solution. To this solution was added a solution of aldehyde in DCE (2 mL) at rt. The resulting yellow solution was stirred for 3 hours, The reaction mixture was quenched by adding aqueous saturated NaHCO₃, and the product was extracted with EtOAc. The EtOAc extract was dried (MgSO₄), and the solvent was removed. The resulting residue was purified by flash chromatography (acetone:hexanes=1:3) to afford (12Z,32R,6S,7R,8E,13E,15E,17S)-17-((tert-butyldimethylsilyl)oxy)-7,15-dimethyl-6-((R)-1-morpholinopropan-2-yl)-15-(trimethylsilyl)-5-oxa-11-aza-1(4,2)-oxazola-3(1,2)-pyrrolidinacycloicosaphane-8,13,15-triene-2,4,10,19-tetraone (16 mg, 49%)

¹H NMR (400 MHz, Chloroform-d) δ 6.50 (dd, J=16.3, 4.1 Hz, 1H), 6.20-6.05 (m, 2H), 5.77 (dd, J=16.3, 2.0 Hz, 1H), 5.56 (ddd, J=14.8, 9.5, 4.1 Hz, 1H), 5.41 (d, J=8.9 Hz, 1H), 5.05-4.95 (m, 2H), 4.80 (dd, J=8.7, 3.3 Hz, 1H), 4.49 (ddd, J=14.1, 9.0, 4.1 Hz, 1H), 3.88 (d, J=17.2 Hz, 1H), 3.81-3.56 (m, 7H), 3.40 (ddd, J=13.5, 9.7, 3.1 Hz, 1H), 2.91 (dd, J=15.8, 5.8 Hz, 1H), 2.95-2.80 (m, 1H), 2.73 (dd, J=15.7, 5.8 Hz, 1H), 2.52-2.29 (m, 6H), 2.29-1.75 (m, 5H), 1.65 (s, 3H), 1.11 (d, J=6.9 Hz, 3H), 0.98 (d, J=6.5 Hz, 3H), 0.85 (s, 9H), 0.30 (s, 9H), 0.05 (s, 3H), 0.02 (s, 3H).

¹³C NMR (100 MHz, CDCl₃) δ 201.0, 171.9, 166.4, 161.8, 161.3, 159.6, 145.0, 144.8, 136.7, 134.7, 132.5, 124.9, 123.6, 79.1, 67.0, 65.4, 62.6, 58.7, 54.1, 50.6, 48.4, 43.7, 41.3, 37.6, 31.7, 28.3, 25.7, 24.8, 18.1, 15.8, 12.7, 10.9, −1.9, −4.5, −5.0.

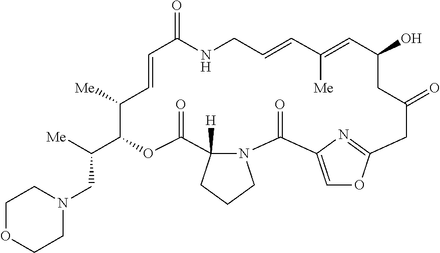 [SMILES: CC1=C\[C@@H](O)CC(=O)Cc2nc(co2)C(=O)N2CCC[C@@H]2C(=O)O[C@H]([C@@H](C)CN2CCOCC2)[C@H](C)/C=C/C(=O)NC\C=C\1]

¹H NMR (400 MHz, Chloroform-d) δ 8.04 (s, 1H), 6.51 (dd, J=16.3, 5.1 Hz, 1H), 6.45 (dd, J=8.9, 3.7 Hz, 1H), 6.09 (d, J=15.6 Hz, 1H), 5.79 (dd, J=16.3, 1.8 Hz, 1H), 5.69 (ddd, J=15.6, 8.8, 4.6 Hz, 1H), 5.34 (d, J=8.8 Hz, 1H), 5.00-4.84 (m, 2H), 4.70 (dd, J=8.9, 3.1 Hz, 1H), 4.45 (ddd, J=14.0, 8.7, 4.6 Hz, 1H), 3.98 (dt, J=11.3, 7.5 Hz, 1H), 3.84 (d, J=15.5 Hz, 1H), 3.79 (d, J=15.7 Hz, 1H), 3.77-3.60 (m, 5H), 3.40 (ddd, J=14.9, 8.9, 3.6 Hz, 1H), 3.05 (dd, J=16.7, 6.4 Hz, 1H), 3.01-2.90 (m, 1H), 2.87 (dd, J=16.8, 5.0 Hz, 1H), 2.52-2.29 (m, 4H), 2.29-1.75 (m, 7H) 1.71 (d, J=1.2 Hz, 3H), 1.06 (d, J=6.8 Hz, 3H), 0.96 (d, J=6.5 Hz, 3H).

¹³C NMR (100 MHz, CDCl₃) δ 202.1, 171.6, 166.6, 160.3, 156.7, 144.3, 143.9, 137.0, 136.7, 134.5, 132.4, 125.4, 124.2, 79.6, 66.9, 65.3, 62.7, 59.7, 54.1, 48.7, 48.43, 43.5, 40.9, 37.5, 31.5, 28.5, 25.0, 15.7, 12.7, 11.4.

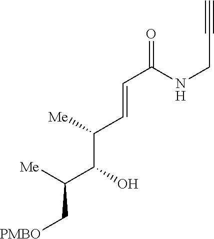

¹H NMR (400 MHz, Chloroform-d) δ 7.23 (d, J=8.6 Hz, 2H), 6.87 (d, J=8.6 Hz, 2H), 6.84 (dd, J=15.5, 1.3 Hz 1H), 5.86 (s, 1H), 5.76 (dd, J=15.5, 1.3 Hz, 1H), 4.42 (s, 2H), 4.09 (dd, J=5.3, 2.6 Hz, 2H), 3.80 (s, 3H), 3.61 (dd, J=9.2, 3.9 Hz, 1H), 3.54-3.39 (m, 3H), 2.49-2.36 (m, 1H), 2.22 (t, J=2.6 Hz, 1H), 1.89 (m, 2H), 1.07 (d, J=6.7 Hz, 3H), 0.92 (d, J=7.0 Hz, 3H).

¹³C NMR (100 MHz, CDCl₃) δ 165.6, 159.3, 148.4, 129.6, 129.39, 129.36, 122.5, 113.8, 99.9, 79.6, 78.6, 77.3, 77.0, 76.7, 74.4, 73.2, 71.5, 55.3, 39.7, 35.6, 29.1, 14.4, 12.9.

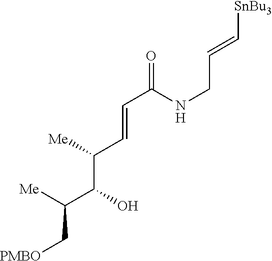

¹H NMR (400 MHz, Chloroform-d) δ 7.23 (d, J=8.7 Hz, 2H), 6.87 (d, J=8.7 Hz, 2H), 6.83 (dd, J=15.5, 7.5 Hz, 1H), 6.11 (dt, J=19.0, 1.4 Hz, 1H), 5.97 (dt, J=19.0, 5.1 Hz, 1H), 5.79 (dd, J=15.5, 1.3 Hz, 1H), 5.54 (br t, J=5.9 Hz, 1H), 4.43 (s, 2H), 4.00-3.93 (m, 2H), 3.80 (s, 3H), 3.62 (dd, J=9.2, 3.9 Hz, 1H), 3.50-3.40 (m, 3H), 2.49-2.37 (m, 1H), 1.95-1.85 (m, 1H), 1.54-1.41 (m, 6H), 1.37-1.24 (m, 6H), 1.08 (d, J=6.7 Hz, 3H), 0.93 (d, J=7.0 Hz, 3H), 0.91-0.77 (m, 15H).

¹³C NMR (100 MHz, CDCl₃) δ 165.6, 159.3, 147.5, 143.5, 130.3, 129.6, 129.4, 123.2, 113.8, 78.7, 74.4, 73.2, 55.3, 44.9, 39.7, 35.6, 29.0, 27.3, 14.4, 13.7, 13.1, 9.4.

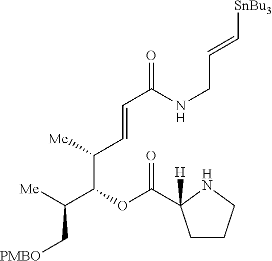

¹H NMR (400 MHz, Chloroform-d) δ 7.24 (d, J=8.7 Hz, 2H), 6.87 (d, J=8.7 Hz, 2H), 6.67 (dd, J=15.5, 7.4 Hz, 1H), 6.11 (dt, J=19.0, 1.5 Hz, 1H), 5.95 (dt, J=19.0, 5.1 Hz, 1H), 5.74 (dd, J=15.5, 1.3 Hz, 1H), 5.54 (br t, J=5.9 Hz, 1H), 4.97 (t, J=6.2 Hz, 1H), 4.37 (s, 2H), 4.03-3.90 (m, 2H), 3.79 (s, 3H), 3.70 (dd, J=8.5, 5.6 Hz, 1H), 3.45 (dd, J=9.2, 5.0 Hz, 1H), 3.21 (dd, J=9.2, 6.4 Hz, 1H), 3.03 (ddd, J=10.2, 7.5, 6.2 Hz, 1H), 2.87 (ddd, J=10.2, 7.0, 6.2 Hz, 1H), 2.80-2.67 (m, 1H), 2.19-2.05 (m, 2H), 1.90-1.64 (m, 3H), 1.57-1.36 (m, 6H), 1.36-1.22 (m, 6H), 1.03 (d, J=6.9 Hz, 3H), 0.95 (d, J=6.9 Hz, 3H), 0.91-0.77 (m, 15H).

¹³C NMR (100 MHz, CDCl₃) δ 175.0, 165.3, 159.1, 145.1, 143.4, 130.4, 130.3, 129.3, 124.2, 113.7, 77.7, 72.8, 71.3, 59.9, 55.2, 46.9, 44.9, 37.8, 35.4, 30.4, 29.0, 27.2, 25.4, 14.8, 13.9, 13.7, 9.4.

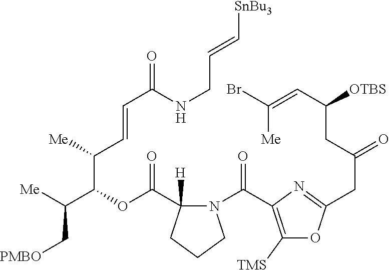

¹H NMR (400 MHz, Chloroform-d) δ 7.26-7.16 (m, 2H), 6.85 (dt, J=8.9, 2.0 Hz, 2H), 6.70-6.52 (m, 1H), 6.18-6.00 (m, 1H), 6.03-5.90 (m, 1H), 5.84-5.70 (m, 2H), 5.68-5.50 (m, 2H), 4.97-4.83 (m, 1H), 4.82-4.65 (m, 1H), 4.59 (td, J=8.6, 3.5 Hz, 1H), 4.40 (s, 1H), 4.31 (s, 1H), 4.15-3.85 (m, 5H), 3.85-3.59 (m, 5H), 3.31-3.10 (m, 1H), 2.89-2.32 (m, 3H), 2.32-2.25 (m, 3H), 2.24-1.70 (m, 2H), 1.60-1.37 (m, 6H), 1.29 (h, J=6.7, 6.1 Hz, 6H), 1.01 (ddd, J=6.9, 5.5, 2.3 Hz, 4H), 0.97-0.71 (m, 26H), 0.42-0.23 (m, 9H), 0.10-0.01 (m, 6H).

¹³C NMR (100 MHz, CDCl₃) δ 201.0, 200.7, 172.3, 172.0, 171.8, 165.4, 165.0, 164.6, 163.8, 163.3, 162.3, 161.5, 161.4, 159.20, 159.08, 159.05, 158.96, 145.2, 145.1, 145.03, 143.5, 143.3, 142.8, 135.0, 134.2, 134.2, 130.9, 130.8, 130.4, 130.3, 130.0, 129.3, 129.3, 129.2, 124.4, 124.3, 121.8, 121.7, 121.0, 113.7, 113.7, 113.6, 78.4, 77.7, 72.7, 72.6, 71.5, 70.9, 67.8, 67.0, 66.1, 60.5, 59.8, 55.2, 55.2, 49.6, 49.5, 48.8, 48.70 47.1, 44.9, 44.8, 44.18, 43.9, 38.1, 37.8, 35.7, 35.6, 31.6, 29.0, 27.2, 25.7, 25.6, 25.2, 24.0, 21.5, 17.9, 14.9, 14.8, 14.76, 14.0, 13.6, 9.4, −1.75, −1.78, −4.6, −5.15, −5.17.

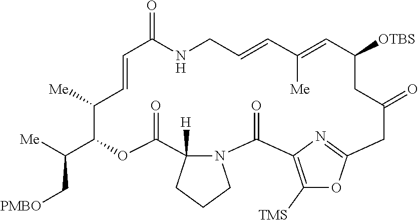

¹H NMR (400 MHz, Chloroform-d) δ 7.27 (d, J=8.4 Hz, 2H), 6.85 (d, J=8.6 Hz, 2H), 6.49 (dd, J=16.4, 4.1 Hz, 1H), 6.14 (dd, J=15.5, 1.3 Hz, 1H), 6.09 (dd, J=9.0, 3.4 Hz, 1H), 5.77 (dd, J=16.4, 2.1 Hz, 1H), 5.57 (ddd, J=15.5, 9.5, 4.2 Hz, 1H), 5.43 (d, J=8.8 Hz, 1H), 5.11 (dd, J=10.5, 1.8 Hz, 1H), 5.02 (ddd, J=9.0, 6.9, 5.8 Hz, 1H), 4.78 (dd, J=8.8, 3.3 Hz, 1H), 4.56-4.36 (m, 3H), 3.89 (d, J=17.1 Hz, 1H), 3.83-3.68 (m, 7H), 3.51 (dd, J=9.1, 3.1 Hz, 1H), 3.39 (ddd, J=14.9, 9.7, 3.5 Hz, 1H), 3.31 (dd, J=9.1, 5.6 Hz, 1H), 2.94 (dd, J=16.1, 7.0 Hz, 1H), 2.76 (dd, J=16.1, 5.8 Hz, 1H), 2.75-2.65 (m, 1H), 2.16-2.10 (m, 2H), 1.91-1.80 (m, 2H), 1.76-1.69 (m, 1H), 1.68 (d, J=1.2 Hz, 3H), 1.08 (d, J=6.9 Hz, 3H), 1.02 (d, J=6.9 Hz, 3H), 0.85 (s, 9H), 0.30 (s, 9H), 0.05 (s, 3H), 0.02 (s, 3H).

¹³C NMR (100 MHz, CDCl₃) δ 200.9, 171.9, 166.5, 161.5, 161.5, 159.5, 158.9, 145.1, 144.5, 136.7, 134.8, 132.4, 130.9, 129.4, 124.9 123.8, 113.6, 76.2, 73.0, 71.6, 65.3, 58.8, 55.2, 50.8, 48.5, 43.5, 41.3, 36.4, 35.4, 28.3, 25.7, 24.9, 18.0, 13.9, 12.7, 9.6, −1.8, −4.5, −5.0.

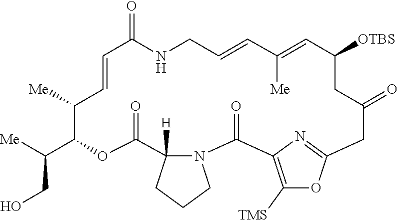 [SMILES: CC1=C\[C@@H](O[Si](C)(C)C(C)(C)C)CC(=O)Cc2nc(c([Si](C)(C)C)o2)C(=O)N2CCC[C@@H]2C(=O)O[C@H]([C@H](C)CO)[C@H](C)/C=C/C(=O)NC\C=C\1]

This primary alcohol can be prepared with above method.

¹H NMR (400 MHz, Chloroform-d) δ 6.48 (dd, J=16.4, 4.4 Hz, 1H), 6.16 (d, J=15.7 Hz, 1H), 6.05 (dd, J=9.0, 2.8 Hz, 1H), 5.75 (dd, J=16.3, 2.0 Hz, 1H), 5.58 (ddd, J=15.6, 9.0, 4.4 Hz, 1H), 5.43 (d, J=8.8 Hz, 1H), 5.09-4.94 (m, 2H), 4.58 (dd, J=8.4, 5.2 Hz, 1H), 4.50 (ddd, J=14.1, 8.7, 4.2 Hz, 1H), 3.91-3.78 (m, 4H), 3.69 (d, J=16.9 Hz, 1H), 3.52 (dd, J=11.6, 3.2 Hz, 1H), 3.40 (ddd, J=15.3, 9.0, 2.8 Hz, 1H), 2.93 (dd, J=16.8, 6.2 Hz, 1H), 2.79 (dd, J=16.9, 5.8 Hz, 1H), 2.79-2.69 (m, 1H), 2.21-2.07 (m, 1H), 2.05-1.95 (m, 1H), 1.90-1.80 (m, 2H), 1.81-1.71 (m, 1H), 1.71 (d, J=1.2 Hz, 3H), 1.07 (d, J=6.9 Hz, 3H), 1.03 (d, J=7.0 Hz, 3H), 0.84 (s, 9H), 0.30 (s, 9H), 0.04 (s, 3H), 0.00 (s, 3H).

¹³C NMR (100 MHz, CDCl₃) δ 200.7, 172.8, 166.9, 162.3, 161.8, 159.8, 144.6, 144.5, 136.8, 135.1, 132.0, 124.2, 123.6, 77.5, 65.0, 64.6, 59.5, 50.8, 49.1, 43.1, 41.1, 36.4, 36.2, 28.1, 25.7, 25.5, 18.0, 14.0, 12.7, 9.4, −1.9, −4.5, −5.0.

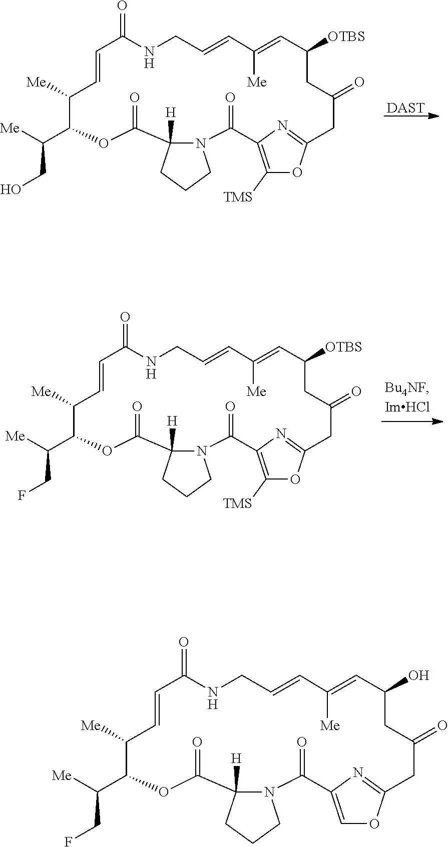

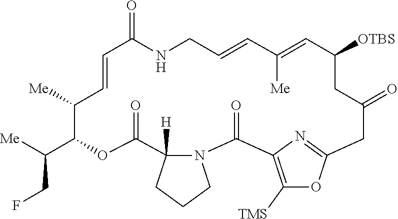 [SMILES: CC1=C\[C@@H](O[Si](C)(C)C(C)(C)C)CC(=O)Cc2nc(c([Si](C)(C)C)o2)C(=O)N2CCC[C@@H]2C(=O)O[C@H]([C@H](C)CF)[C@H](C)/C=C/C(=O)NC\C=C\1]

To a solution of (12Z,32R,6S,7R,8E,13E,15E,17S)-17-((tert-butyldimethylsilyl)oxy)-6-((R)-1-hydroxypropan-2-yl)-7,15-dimethyl-15-(trimethylsilyl)-5-oxa-11-aza-1(4,2)-oxazola-3(1,2)-pyrrolidinacycloicosaphane-8,13,15-triene-2,4,10,19-tetraone (0.025 g, 1 Eq, 34 μmol) in DCM (5 mL) was added DAST (14 mg, 12 μL, 2.600 Eq, 89 μmol) drop wise at 0° C. under N₂. The reaction was warmed to RT and stirred for 3 hours. The reaction mixture was quenched with aqueous sat. NaHCO₃ solution, diluted with 20 mL DCM and transferred to a separate funnel. The organic solution was washed with water and brine. The washed solution was dried with Na₂SO₄ and the dried solution was concentrated under vacuum. The residue was purified by flash chromatography (Acetone:Hexanes=1:5 to 1:2) to afford (12Z,32R,6S,7R,8E,13E,15E,17S)-17-((tert-butyldimethylsilyl)oxy)-6-((S)-1-fluoropropan-2-yl)-7,15-dimethyl-15-(trimethylsilyl)-5-oxa-11-aza-1(4,2)-oxazola-3(1,2)-pyrrolidinacycloicosaphane-8,13,15-triene-2,4,10,19-tetraone (0.013 g, 52%) as a white solid.

1H NMR (400 MHz, Chloroform-d) δ 6.48 (dd, J=16.4, 4.1 Hz, 1H), 6.14 (d, J=15.7 Hz, 1H), 6.09 (dd, J=8.8, 2.6 Hz, 1H), 5.79 (dd, J=16.3, 2.0 Hz, 1H), 5.57 (ddd, J=15.5, 9.3, 4.3 Hz, 1H), 5.42 (d, J=8.9 Hz, 1H), 5.07 (dd, J=10.5, 1.8 Hz, 1H), 5.01 (dt, J=8.9, 6.4 Hz, 1H), 4.75 (dd, J=8.9, 3.5 Hz, 1H), 4.57-4.41 (m, 2H), 4.38 (ddd, J=47.5, 9.2, 5.2 Hz 1H), 3.88 (d, J=17.1 Hz, 1H), 3.81-3.70 (m, 2H), 3.73 (d, J=17.1 Hz, 1H), 3.39 (ddd, J=14.9, 9.4, 3.2 Hz, 1H), 2.91 (dd, J=16.1, 6.8 Hz, 1H), 2.80-2.69 (m, 1H), 2.91 (dd, J=16.0, 5.9 Hz, 1H), 2.20-2.05 (m, 2H), 1.90-1.80 (m, 3H), 1.67 (d, J=1.2 Hz, 3H), 1.11 (d, J=6.9 Hz, 3H), 1.08 (d, J=6.9 Hz, 3H), 0.85 (s, 9H), 0.31 (s, 9H), 0.05 (s, 3H), 0.02 (s, 3H).

13C NMR (100 MHz, CDCl3) δ 201.0, 172.1, 166.3, 161.8, 161.7, 159.7, 144.9, 144.0, 136.7, 134.9, 132.3, 124.7, 124.1, 85.05 (d, JCF=169.7 Hz), 75.1 (d, JCF=5.2 Hz), 65.3, 58.8, 50.6, 48.5, 43.6, 41.3, 36.3, 35.8 (d, JCF=19.2 Hz), 28.2, 25.7, 25.0, 18.1, 12.9 (d, JCF=4.7 Hz), 12.7, 9.7, −1.9, −4.50, −4.96.

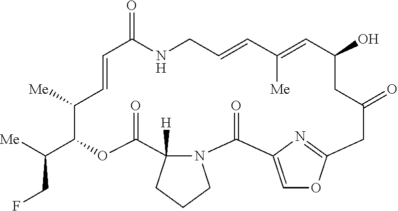 [SMILES: CC1=C\[C@@H](O)CC(=O)Cc2nc(co2)C(=O)N2CCC[C@@H]2C(=O)O[C@H]([C@H](C)CF)[C@H](C)/C=C/C(=O)NC\C=C\1]

1H NMR (400 MHz, Chloroform-d) δ 8.11 (s, 1H), 6.49 (dd, J=16.4, 5.0 Hz, 1H), 6.48-6.40 (m, 1H), 6.13 (d, J=15.7 Hz, 1H), 5.83 (d, J=16.4 Hz, 1H), 5.72 (ddd, J=14.8, 9.1, 4.6 Hz, 1H), 5.42 (d, J=8.8 Hz, 1H), 5.03 (d, J=10.4 Hz, 1H), 4.93 (dt, J=9.6, 5.6 Hz, 1H), 4.69 (dd, J=8.9, 3.2 Hz, 1H), 4.55-4.30 (m, 3H), 4.01 (dt, J=11.5, 7.2 Hz, 1H), 3.84 (s, 2H), 3.81-3.71 (m, 1H), 3.41 (ddd, J=13.8, 9.2, 3.7 Hz, 1H), 3.07 (dd, J=17.0, 5.9 Hz, 1H), 3.03-2.95 (m, 1H), 2.90 (dd, J=17.0, 5.1 Hz, 1H), 2.75 (br t, J=6.6 Hz, 1H), 2.25-2.11 (m, 2H), 2.00-1.84 (m, 3H), 1.74 (s, 3H), 1.09 (d, J=6.9 Hz, 6H).

13C NMR (100 MHz, CDCl3) δ 202.3, 171.6, 166.5, 160.2, 156.8, 144.0, 143.6, 137.0, 134.4, 132.6, 125.3, 125.3, 124.6, 85.3 (d, JCF=170.4 Hz), 76.1 (d, JCF=4.8 HZ), 65.2, 59.6, 52.1, 48.8, 48.5, 43.3, 40.9, 36.4, 35.8 (d, J_{CF}=19.1 Hz), 29.7, 28.3, 25.13, 25.11, 20.2, 13.5, 13.0 (d, J_{CF}=5.2 Hz), 12.7, 10.1.

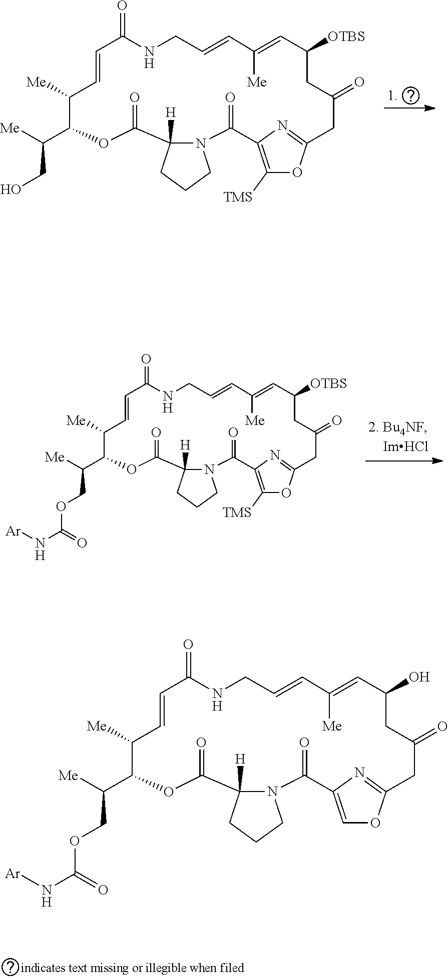

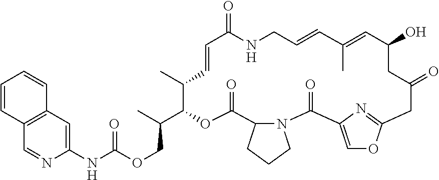 [SMILES: CC1=C\[C@@H](O)CC(=O)Cc2nc(co2)C(=O)N2CCCC2C(=O)O[C@H]([C@H](C)COC(=O)Nc2cc3ccccc3cn2)[C@H](C)/C=C/C(=O)NC\C=C\1]

To a solution of (12Z,32R,6S,7R,8E,13E,15E,17S)-17-((tert-butyldimethylsilyl)oxy)-6-((R)-1-hydroxypropan-2-yl)-7,15-dimethyl-15-(trimethylsilyl)-5-oxa-11-aza-1(4,2)-oxazola-3(1,2)-pyrrolidinacycloicosaphane-8,13,15-triene-2,4,10,19-tetraone (0.100 g, 1 Eq, 137 μmol) and DMAP (1.67 mg, 0.1 Eq, 13.7 μmol) was added 3-isocyanatoisoquinoline (117 mg, 14 mL, 5 Eq, 685 μmol) under N₂. The resulting yellow solution was heated at 80° C. for 3 hours. The solvent was removed and the residue was purified by flash chromatography (acetone:Hexanes=1:6) to afford (R)-2-((12Z,32R,6S,7R,8E,13E,15E,17S)-17-((tert-butyldimethylsilyl)oxy)-7,15-dimethyl-2,4,10,19-tetraoxo-15-(trimethylsilyl)-5-oxa-11-aza-1(4,2)-oxazola-3(1,2)-pyrrolidinacycloicosaphane-8,13,15-trien-6-yl)propyl isoquinolin-3-ylcarbamate (0.064 g, 52%) as a white solid.

Then the analogues were obtained by deprotection of TBS and TMS with TBAF and imidazole HCl (84% yield).

| | | MS- |
|---|---|---|
| | | ESI |
| | | [M + |
| Compound and ID | NMR DATA | 1] |
| 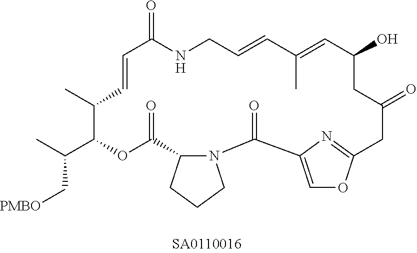 | ¹H NMR (400 MHz, CDCl₃) δ 8.06 (s, 1H), 7.21 (d, J = 8.6 Hz, 2H), 6.86 (d, J = 8.6 Hz, 2H), 6.48 (dd, J = 16.3, 5.0 Hz, 1H), 6.41 (dd, J = 8.9, 3.6 Hz, 1H), 6.09 (d, J = 15.6 Hz, 1H), 5.76 (dd, J = 16.4, 1.8 Hz, 1H), 5.72-5.63 (m, 1H), 5.36 (d, J = 8.7 Hz, 1H), 5.01 (dd, J = 9.1, 2.0 Hz, 1H), 4.90 (dt, J = 8.9, 5.8 Hz, 1H), 4.69 (dd, J = 9.0, 3.0 Hz, 1H), 4.52-4.32 (m, 3H), 3.97 (dt, J = 11.2, 7.6 Hz, 1H), 3.86- 3.68 (m, 7H), 3.44-3.30 (m, 3H), 3.04 (dd, J = 16.8, 6.2 Hz, 1H), 2.87 (dd, J = 16.8, 5.1 Hz, 1H), 2.76 (ddd, J = 9.1, 4.6, 2.0 Hz, 1H), 2.22- | 664.3 |
| | 2.04 (m, 2H), 1.91 (m, 2H), 1.81 | |
| | (m, 1H), 1.71 (d, J = 1.2 Hz, 3H), | |
| | 1.03 (d, J = 6.8 Hz, 3H), 1.01 (d, J = | |
| | 6.7 Hz, 3H). ¹³C NMR (100 MHz, | |
| | CDCl₃) δ 202.2, 171.6, 166.7, | |
| | 160.3, 159.2, 156.8, 144.4, 143.9, | |
| | 137.0, 136.7, 134.4, 132.4, 130.1, | |
| | 129.1, 125.4, 124.1, 113.8, 78.5, | |
| | 72.9, 71.9, 65.2, 59.6, 55.3, 48.7, | |
| | 48.41, 43.4, 40.9, 37.2, 34.9, 28.4, | |
| | 25.0, 14.7, 12.7, 11.1. | |
| 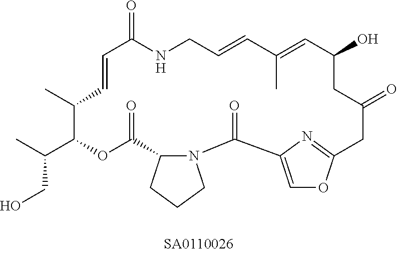 [SMILES: CC1=C\[C@@H](O)CC(=O)Cc2nc(co2)C(=O)N2CCC[C@@H]2C(=O)O[C@H]([C@@H](C)CO)[C@H](C)/C=C/C(=O)NC\C=C\1] | ¹H NMR (300 MHz, CDCl₃) δ 8.07 (s, 1H), 6.52 (dd, J = 16.3, 5.1 Hz, 1H), 6.60-6.45 (m, 1H), 6.09 (d, J = 15.7 Hz, 1H), 5.87-5.75 (m, 1H), 5.77-5.63 (m, 1H), 5.36 (d, J = 8.7 Hz, 1H), 5.10 (dd, J = 8.3, 2.1 Hz, 1H), 5.00-4.85 (m, 1H), 4.75- 4.58 (m, 1H), 4.45 (dd, J = 12.1, 7.5 Hz, 1H), 3.99 (dd, J = 11.7, 7.4 Hz, 1H), 3.85-3.75 (m, 1H), 3.81 (s, 2H), 3.59 (dd, J = 5.3, 2.3 Hz, 2H), 3.45-3.31 (m, 2H), 3.03 (dd, J = 17.6, 6.1 Hz, 1H), 2.88 (dd, J = 17.6, 6.1 Hz, 1H), 2.88-2.78 (m, 1H), 2.60 (br s, 1H), 2.33-2.11 (m, 1H), 2.10-1.80 (m, 4H), 1.71 (d, J = | 544.3 |
| | 1.2 Hz, 3H), 1.09 (d, J = 6.8 Hz, | |
| | 3H), 1.01 (d, J = 6.8 Hz, 6H) | |
| 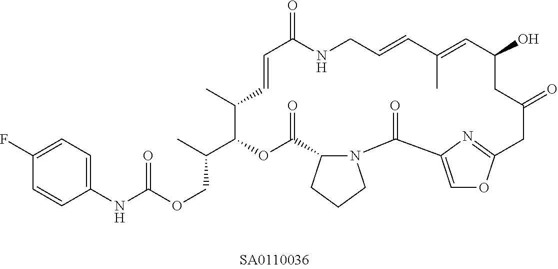 [SMILES: CC1=C\[C@@H](O)CC(=O)Cc2nc(co2)C(=O)N2CCC[C@@H]2C(=O)O[C@H]([C@@H](C)COC(=O)Nc2ccc(F)cc2)[C@H](C)/C=C/C(=O)NC\C=C\1] | ¹H NMR (400 MHz, CDCl₃) δ 8.04 (s, 1H), 7.73 (br s, 1H), 7.42 (br s, 2H), 6.98 (t, J = 8.7 Hz, 2H), 6.50 (dd, J = 16.3, 4.9 Hz, 1H), 6.19 (d, J = 8.6 Hz, 1H), 6.11 (d, J = 15.6 Hz, 1H), 5.79 (dd, J = 16.3, 1.8 Hz, 1H), 5.69 (ddd, J = 15.5, 9.0, 4.5 Hz, 1H), 5.43 (d, J = 8.7 Hz, 1H), 5.19 (d, J = 3.0 Hz, 1H), 4.95-4.85 (m, 1H), 4.74 (dd, J = 8.9, 3.0 Hz, 1H), 4.43 (ddd, J = 14.1, 8.9, 4.7 Hz, 1H), 4.25 (dd, J = 10.9, 3.4 Hz, 1H), 4.07 (dd, J = 10.9, 4.0 Hz, 1H), 3.98 (dt, J = 11.4, 7.5 Hz, 1H), 3.82 (s, 2H), 3.80-3.72 (m, 1H), 3.41 (ddd, J = 15.0, 9.1, 3.8 Hz, 1H), 3.04 (dd, J = 17.2, 5.6 Hz, 1H), 2.90 | 681.2 |
| | (dd, J = 17.2, 5.5 Hz, 1H), 2.79- | |
| | 2.72 (m, 1H), 2.60 (br s, 1H), 2.26- | |
| | 2.12 (m, 2H), 1.94 (dp, J = 11.2, 3.8 | |
| | Hz, 2H), 1.87-1.77 (m, 1H), 1.73 | |
| | (d, J = 1.2 Hz, 3H), 1.14 (d, J = 6.6 | |
| | Hz, 6H). | |
| 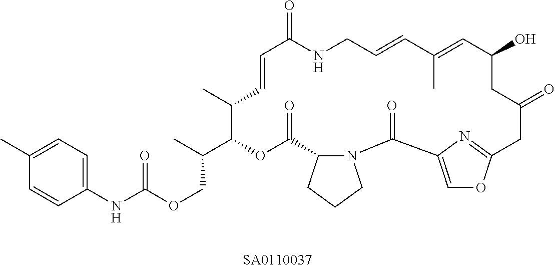 [SMILES: CC1=C\[C@@H](O)CC(=O)Cc2nc(co2)C(=O)N2CCC[C@@H]2C(=O)O[C@H]([C@@H](C)COC(=O)Nc2ccc(C)cc2)[C@H](C)/C=C/C(=O)NC\C=C\1] | ¹H NMR (400 MHz, CDCl₃) δ 8.05 (s, 1H), 7.48 (br s, 1H), 7.31 (d, J = 8.0 Hz, 2H), 7.08 (d, J = 8.1 Hz, 2H), 6.50 (dd, J = 16.3, 5.0 Hz, 1H), 6.33 (br s, 1H), 6.10 (d, J = 15.6 Hz, 1H), 5.79 (dd, J = 16.3, 1.8 Hz, 1H), 5.68 (ddd, J = 15.6, 8.9, 4.5 Hz, 1H), 5.41 (d, J = 8.7 Hz, 1H), 5.14 (d, J = 6.5 Hz, 1H), 4.91 (dt, J = 8.8, 5.7 Hz, 1H), 4.73 (dd, J = 9.0, 3.1 Hz, 1H), 4.42 (ddd, J = 14.1, 8.6, 4.7 Hz, 1H), 4.21 (dd, J = 11.0, 3.8 Hz, 1H), 4.07 (dd, J = 11.0, 4.3 Hz, 1H), 3.97 (dt, J = 11.4, 7.5 Hz, 1H), 3.87-3.72 (m, 1H), 3.81 (s, 2H), 3.46-3.36 (m, 1H), 3.03 (dd, J = 17.0, 5.8 Hz, 1H), 2.89 (dd, J = | 677.4 |
| | 17.0, 5.5 Hz, 1H), 2.76 (td, J = 6.2, | |
| | 5.3, 2.7 Hz, 1H), 2.29 (s, 3H), 2.24- | |
| | 2.10 (m, 2H), 1.99-1.89 (m, 2H), | |
| | 1.85-1.75 (m, 1H), 1.71 (d, J = 1.2 | |
| | Hz, 3H), 1.12 (d, J = 7.0 Hz, 3H), | |
| | 1.11 (d, J = 6.9 Hz, 3H). | |
| 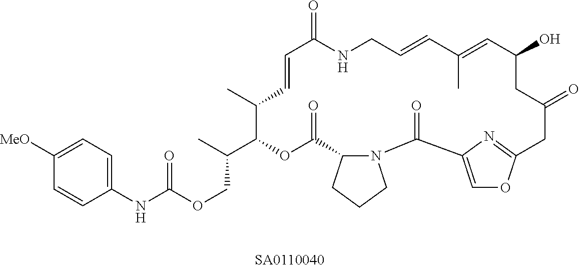 [SMILES: COc1ccc(NC(=O)OC[C@H](C)[C@H]2OC(=O)[C@H]3CCCN3C(=O)c3coc(n3)CC(=O)C[C@H](O)/C=C(C)/C=C/CNC(=O)/C=C/[C@H]2C)cc1] | ¹H NMR (400 MHz, CDCl₃) δ 8.04 (br s, 1H), 7.43 (s, 1H), 7.34 (d, J = 8.0 Hz, 2H), 6.83 (d, J = 9.0 Hz, 2H), 6.50 (d, J = 16.4 Hz, 1H), 6.28 (s, 1H), 6.10 (d, J = 15.6 Hz, 1H), 5.79 (d, J = 16.3 Hz, 1H), 5.69 (ddd, J = 15.5, 8.9, 4.5 Hz, 1H), 5.41 (d, J = 8.7 Hz, 1H), 5.16 (s, 1H), 4.91 (dt, J = 8.6, 5.6 Hz, 1H), 4.80-4.67 (m, 1H), 4.43 (ddd, J = 14.0, 8.7, 4.6 Hz, 1H), 4.21 (dd, J = 11.0, 3.7 Hz, 1H), 4.07 (dd, J = 11.0, 4.3 Hz, 1H), 3.98 (dt, J = 11.3, 7.5 Hz, 1H), 3.81 (s, 2H), 3.78 (s, 3H), 3.41 (ddd, J = 14.6, 8.9, 3.7 Hz, 1H), 3.03 (dd, J = 17.1, 5.8 Hz, 1H), 2.89 (dd, J = 17.1, 5.4 Hz, 1H), 2.76 (s, 1H), 2.64 | 693.4 |
| | (s, 1H), 2.25-2.10 (m, 2H), 2.00- | |
| | 1.83 (m, 2H), 1.87-1.79 (m, 1H), | |
| | 1.72 (d, J = 1.2 Hz, 3H), 1.12 (d, J = | |
| | 6.8 Hz, 6H). | |
| 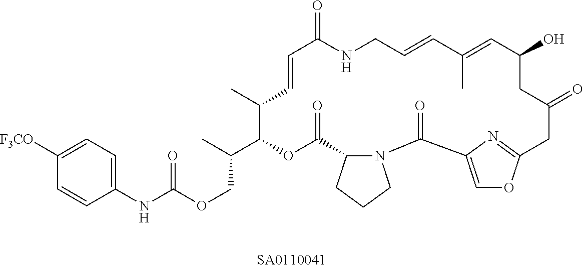 [SMILES: CC1=C\[C@@H](O)CC(=O)Cc2nc(co2)C(=O)N2CCC[C@@H]2C(=O)O[C@H]([C@@H](C)COC(=O)Nc2ccc(OC(F)(F)F)cc2)[C@H](C)/C=C/C(=O)NC\C=C\1] | ¹H NMR (400 MHz, CDCl₃)) δ 8.05 (s, 1H), 7.92 (br s, 1H), 7.51 (d, J = 8.7 Hz, 2H), 7.13 (d, J = 8.6 Hz, 2H), 6.50 (dd, J = 16.3, 4.8 Hz, 1H), 6.16 (s, 1H), 6.12 (d, J = 15.6 Hz, 1H), 5.79 (dd, J = 16.3, 1.8 Hz, 1H), 5.75-5.63 (m, 1H), 5.45 (d, J = 8.7 Hz, 1H), 5.22 (d, J = 4.8 Hz, 1H), 4.91 (q, J = 7.6, 6.6 Hz, 1H), 4.75 (dd, J = 9.0, 3.0 Hz, 1H), 4.43 (ddd, J = 14.0, 8.7, 4.6 Hz, 1H), 4.30 (dd, J = 10.9, 3.3 Hz, 1H), 4.07 (dd, J = 10.9, 3.9 Hz, 1H), 3.98 (dt, J = 11.4, 7.5 Hz, 2H), 3.83 (s, 2H), 3.83- 3.74 (m, 1H), 3.41 (ddd, J = 13.7, 9.0, 3.7 Hz, 1H), 3.04 (dd, J = 17.3, 5.6 Hz, 1H), 2.90 (dd, J = 17.2, 5.5 | 747.4 |
| | Hz, 1H), 2.79-2.69 (m, 1H), 2.56 | |
| | (br s, 1H), 2.30-2.13 (m, 2H), 2.00- | |
| | 1.88 (m, 2H), 1.87-1.75 (m, 1H), | |
| | 1.73 (d, J = 1.2 Hz, 3H), 1.16 (d, J = | |
| | 2.8 Hz, 3H), 1.14 (d, J = 2.9 Hz, | |
| | 3H). | |
| 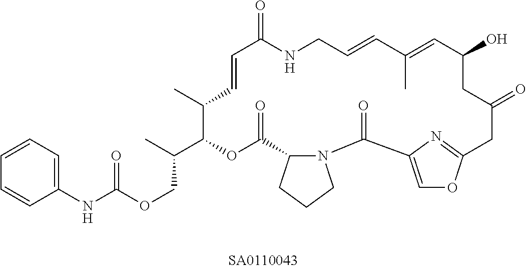 [SMILES: CC1=C\[C@@H](O)CC(=O)Cc2nc(co2)C(=O)N2CCC[C@@H]2C(=O)O[C@H]([C@@H](C)COC(=O)Nc2ccccc2)[C@H](C)/C=C/C(=O)NC\C=C\1] | ¹H NMR (400 MHz, CDCl₃) δ 8.06 (s, 1H), 7.54 (br s, 1H), 7.45 (d, J = 8.0 Hz, 2H), 7.36-7.26 (m, 2H), 7.05 (tt, J = 7.4, 1.2 Hz, 1H), 6.50 (dd, J = 16.3, 4.9 Hz, 1H), 6.24 (dd, J = 8.2, 2.9 Hz, 1H), 6.11 (d, J = 15.7 Hz, 1H), 5.79 (dd, J = 16.3, 1.8 Hz, 1H), 5.69 (ddd, J = 15.6, 9.0, 4.5 Hz, 1H), 5.42 (d, J = 8.6 Hz, 1H), 5.17 (d, J = 4.3 Hz, 1H), 4.91 (d, J = 7.6 Hz, 1H), 4.74 (dd, J = 9.0, 3.1 Hz, 1H), 4.44 (dd, J = 13.9, 8.7, 4.6 Hz, 1H), 4.24 (dd, J = 11.0, 3.7 Hz, 1H), 4.08 (dd, J = 10.9, 4.3 Hz, 1H), 3.99 (dt, J = 11.2, 7.4 Hz, 1H), 3.82 (s, 2H), 3.81- 3.73 (m, 1H), 3.40 (ddd, J = 14.5, | 662.3 |
| | 9.1, 3.8 Hz, 1H), 3.04 (dd, J = 17.2, | |
| | 5.7 Hz, 1H), 2.89 (dd, J = 17.2, 5.4 | |
| | Hz, 1H), 2.81-2.71 (m, 1H), 2.58 | |
| | (br s, 1H), 2.20 (td, J = 8.9, 3.7 Hz, | |
| | 2H), 1.94 (ddt, J = 11.9, 7.3, 3.7 Hz, | |
| | 2H), 1.88-1.76 (m, 1H), 1.72 (d, J = | |
| | 1.2 Hz, 3H), 1.14 (d, J = 6.8 Hz, | |
| | 6H). | |
| 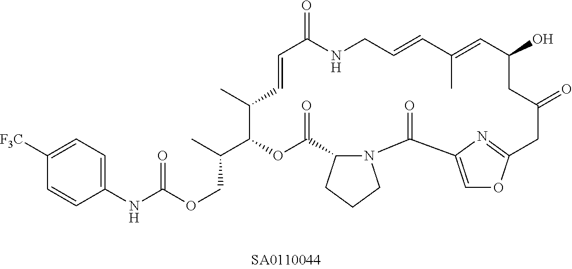 [SMILES: CC1=C\[C@@H](O)CC(=O)Cc2nc(co2)C(=O)N2CCC[C@@H]2C(=O)O[C@H]([C@@H](C)COC(=O)Nc2ccc(C(F)(F)F)cc2)[C@H](C)/C=C/C(=O)NC\C=C\1] | ¹H NMR (400 MHz, CDCl₃) δ 8.18 (br s, 1H), 8.06 (s, 1H), 7.62 (d, J = 8.5 Hz, 2H), 7.52 (d, J = 8.7 Hz, 2H), 6.50 (dd, J = 16.3, 4.9 Hz, 1H), 6.18-6.10 (m, 1H), 6.12 (d, J = 15.4 Hz, 1H), 5.80 (dd, J = 16.3, 1.8 Hz, 1H), 5.69 (ddd, J = 15.5, 9.1, 4.4 Hz, 1H), 5.46 (d, J = 8.7 Hz, 1H), 5.25 (dd, J = 4.7, 2.2 Hz, 1H), 4.92 (d, J = 7.7 Hz, 1H), 4.75 (dd, J = 9.0, 3.1 Hz, 1H), 4.42 (ddd, J = 13.7, 8.5, 4.4 Hz, 1H), 4.33 (dd, J = 10.9, 3.2 Hz, 1H), 4.08 (dd, J = 11.0, 3.7 Hz, 1H), 4.04-3.91 (m, 1H), 3.83 (s, 2H), 3.82-3.76 (m, 1H), 3.42 (ddd, J = 15.0, 9.1, 3.8 Hz, 1H), 3.04 (dd, J = 17.3, 5.6 Hz, | 730.2 |
| | 1H), 2.91 (dd, J = 17.3, 5.5 Hz, 1H), | |
| | 2.79-2.69 (m, 1H), 2.60 (br s, 1H), | |
| | 2.24-2.14 (m, 2H), 2.00-1.90 (m, | |
| | 2H), 1.85-1.76 (m, 1H), 1.73 (d, J = | |
| | 1.2 Hz, 3H), 1.17 (d, J = 4.2 Hz, | |
| | 3H), 1.15 (d, J = 4.2 Hz, 3H). | |
| 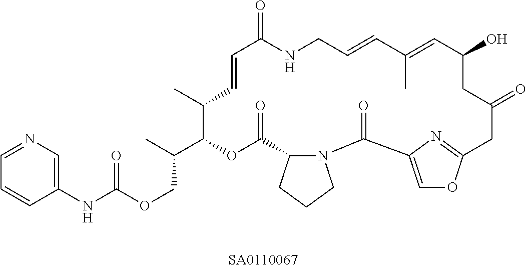 [SMILES: CC1=C\[C@@H](O)CC(=O)Cc2nc(co2)C(=O)N2CCC[C@@H]2C(=O)O[C@H]([C@@H](C)COC(=O)Nc2cccnc2)[C@H](C)/C=C/C(=O)NC\C=C\1] | ¹H NMR (400 MHz, CDCl₃) δ 8.52 (d, J = 2.6 Hz, 1H), 8.37 (br s, 1H), 8.24 (dd, J = 4.7, 1.5 Hz, 1H), 8.09 (br s, 1H), 8.08 (s, 1H), 7.23 (dd, J = 8.4, 4.8 Hz, 1H), 6.49 (dd, J = 16.3, 4.8 Hz, 1H), 6.15 (d, J = 9.5 Hz, 1H), 6.11 (d, J = 15.8 Hz, 1H), 5.80 (dd, J = 16.3, 1.8 Hz, 1H), 5.68 (ddd, J = 15.7, 8.7, 4.3 Hz, 1H), 5.46 (d, J = 8.7 Hz, 1H), 5.25 (dd, J = 4.2, 2.2 Hz, 1H), 4.91 (dt, J = 8.7, 5.7 Hz, 1H), 4.76 (dd, J = 9.0, 3.2 Hz, 1H), 4.41 (ddd, J = 13.7, 8.1, 4.0 Hz, 1H), 4.26 (dd, J = 10.9, 2.9 Hz, 1H), 4.15 (dd, J = 10.9, 3.7 Hz, 1H), 3.98 (dt, J = 11.5, 7.4 Hz, 1H), 3.88 (td, J = 7.5, 7.0, 3.8 Hz, 1H), | 663.2 |
| | 3.82 (s, 2H), 3.44 (ddd, J = 15.2, | |
| | 8.7, 3.8 Hz, 1H), 3.07-2.91 (m, | |
| | 2H), 2.76-2.66 (m, 1H), 2.28- | |
| | 2.08 (m, 2H), 2.00-1.90 (m, 2H), | |
| | 1.89-1.75 (m, 1H), 1.72 (d, J = 1.2 | |
| | Hz, 3H), 1.18 (d, J = 7.9 Hz, 3H), | |
| | 1.16 (d, J = 7.4 Hz, 3H). ¹³C NMR | |
| | (100 MHz, CDCl₃) δ 202.4, 171.1, | |
| | 166.4, 160.4, 157.4, 154.1, 144.2, | |
| | 144.0, 143.8, 140.8, 137.0, 136.1, | |
| | 135.6, 134.2, 132.8, 126.3, 125.2, | |
| | 124.4, 123.6, 76.2, 69.3, 65.1, 59.7, | |
| | 48.8, 48.7, 43.1, 41.0, 40.1, 35.5, | |
| | 28.5, 25.0, 13.5, 12.7, 11.0. | |
| 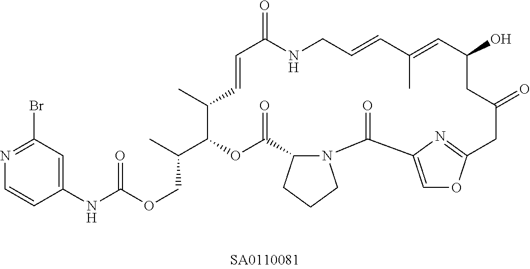 [SMILES: CC1=C\[C@@H](O)CC(=O)Cc2nc(co2)C(=O)N2CCC[C@@H]2C(=O)O[C@H]([C@@H](C)COC(=O)Nc2ccnc(Br)c2)[C@H](C)/C=C/C(=O)NC\C=C\1] | ¹H NMR (400 MHz, CDCl₃) δ 8.91 (s, 1H), 8.20-8.10 (m, 2H), 7.71 (d, J = 1.9 Hz, 1H), 7.63-7.52 (m, 1H), 6.50 (dd, J = 16.2, 4.7 Hz, 1H), 6.13 (d, J = 15.8 Hz, 2H), 6.09 (d, J = 3.8 Hz, 1H), 5.78 (dd, J = 16.2, 1.9 Hz, 1H), 5.73-5.65 (m, 1H), 5.50 (d, J = 8.7 Hz, 1H), 5.30 (t, J = 2.7 Hz, 1H), 4.92 (dt, J = 8.7, 5.5 Hz, 1H), 4.75 (dd, J = 8.9, 3.1 Hz, 1H), 4.47-4.36 (m, 1H), 4.34 (dd, J = 10.8, 2.6 Hz, 1H), 4.09 (dd, J = 10.9, 3.1 Hz, 1H), 3.99-3.85 (m, 2H), 3.84 (s, 2H), 3.45 (ddd, J = 14.2, 9.1, 4.0 Hz, 1H), 3.04 (dd, J = 17.3, 5.6 Hz, 1H), 2.93 (dd, J = 17.2, 5.5 Hz, 1H), 2.75-2.65 (m, | 741.2 |
| | 1H), 2.27-2.08 (m, 2H), 1.99- | |
| | 1.89 (m, 2H), 1.87-1.76 (m, 1H), | |
| | 1.73 (d, J = 1.2 Hz, 6H), 1.17 (d, J = | |
| | 6.9 Hz, 6H). ¹³C NMR (100 MHz, | |
| | CDCl₃) δ 202.4, 170.8, 166.3, | |
| | 160.4, 157.5, 153.2, 150.3, 148.1, | |
| | 144.0, 143.9 142.4, 137.0, 136.5, | |
| | 134.3, 132.8, 125.2, 124.2, 116.4, | |
| | 112.1, 76.0, 70.3, 65.0, 59.6, 49.0, | |
| | 48.8, 42.8, 41.2, 40.7, 35.5, 28.5, | |
| | 24.94 13.1 12.7, 11.0. | |
| 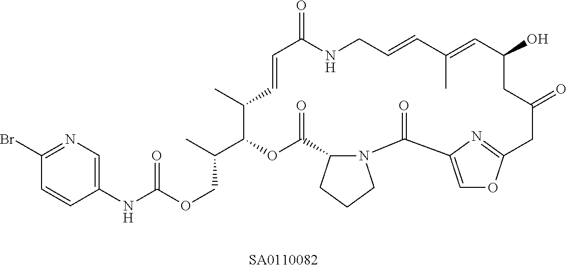 [SMILES: CC1=C\[C@@H](O)CC(=O)Cc2nc(co2)C(=O)N2CCC[C@@H]2C(=O)O[C@H]([C@@H](C)COC(=O)Nc2ccc(Br)nc2)[C@H](C)/C=C/C(=O)NC\C=C\1] | ¹H NMR (400 MHz, CDCl₃) δ 8.66 (br s, 1H), 8.41 (d, J = 2.8 Hz, 1H), 8.15-7.95 (m, 1H), 8.07 (s, 1H), 7.23 (d, J = 8.7 Hz, 1H), 6.51 (dd, J = 16.2, 4.8 Hz, 1H), 6.19 (br s, 1H), 6.11 (d, J = 15.6 Hz, 1H), 5.79 (dd, J = 16.3, 1.8 Hz, 1H), 5.67 (ddd, J = 15.6, 8.9, 4.4 Hz, 1H), 5.46 (d, J = 8.7 Hz, 1H), 5.27 (dd, J = 3.9, 2.2 Hz, 1H), 4.91 (dt, J = 9.1, 5.7 Hz, 1H), 4.76 (dd, J = 8.9, 3.1 Hz, 1H), 4.37 (ddd, J = 14.0, 8.1, 4.3 Hz, 1H), 4.30 (dd, J = 11.0, 2.7 Hz, 1H), 4.09 (dd, J = 10.9, 3.4 Hz, 1H), 3.93 (dt, J = 11.7, 7.6 Hz, 1H), 3.89-3.78 (m, 1H), 3.82 (s, 2H), 3.46 (ddd, J = 15.0, 9.0, 4.0 Hz, | 741.2 |
| | 1H), 3.02 (dd, J = 17.1, 5.6 Hz, 1H), | |
| | 2.97-2.83 (m, 2H), 2.75-2.58 (m, | |
| | 1H), 2.30-2.10 (m, 1H), 2.00- | |
| | 1.86 (m, 2H), 1.84-1.74 (m, 2H), | |
| | 1.72 (d, J = 1.2 Hz, 3H), 1.16 (dd, J = | |
| | 6.9, 3.5 Hz, 6H). ¹³C NMR (100 | |
| | MHz, CDCl₃) δ 202.4, 170.9, 166.3, | |
| | 160.4, 157.4, 153.9, 144.0, 144.0, | |
| | 140.2, 136.9, 136.4, 134.9, 134.3, | |
| | 132.8, 128.9, 127.7, 125.2, 124.2, | |
| | 124.0, 76.2, 69.6, 65.0, 59.6, 48.9, | |
| | 48.7, 42.9, 41.1, 40.2, 35.5, 28.4, | |
| | 24.9, 13.3, 12.7, 11.0. | |
| 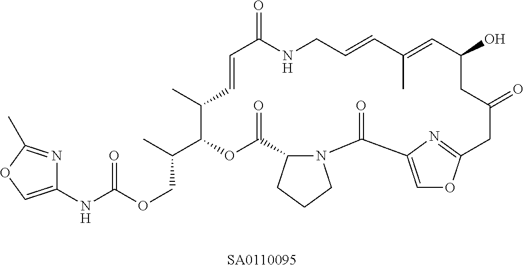 [SMILES: CC1=C\[C@@H](O)CC(=O)Cc2nc(co2)C(=O)N2CCC[C@@H]2C(=O)O[C@H]([C@@H](C)COC(=O)Nc2coc(C)n2)[C@H](C)/C=C/C(=O)NC\C=C\1] | ¹H NMR (400 MHz, CDCl₃) δ 8.89 (s, 1H), 8.65 (s, 1H), 7.67 (s, 1H), 6.46 (dd, J = 16.4, 4.8 Hz, 1H), 6.21-6.07 (m, 2H), 5.78 (dd, J = 16.4, 1.9 Hz, 1H), 5.68 (ddd, J = 15.6, 9.2, 4.5 Hz, 1H), 5.47 (d, J = 8.6 Hz, 1H), 5.24-5.13 (m, 1H), 4.91 (dt, J = 8.9, 5.3 Hz, 1H), 4.71 (dd, J = 8.7, 3.2 Hz, 1H), 4.47 (ddd, J = 13.9, 9.0, 4.6 Hz, 1H), 4.18 (dd, J = 10.7, 2.9 Hz, 1H), 4.15-3.99 (m, 2H), 3.90-3.75 (m, 3H), 3.35 (ddd, J = 13.8, 9.1, 3.5 Hz, 1H), 3.04 (dd, J = 17.7, 5.0 Hz, 1H), 2.91 (dd, J = 17.6, 5.7 Hz, 1H), 2.2.80- 2.60 (m, 2H), 2.40 (s, 3H), 2.27- 2.14 (m, 2H), 1.98-1.78 (m, 3H), | 667 |
| | 1.73 (d, J = 1.2 Hz, 3H), 1.16 (d, J = | |
| | 7.0 Hz, 3H), 1.14 (d, J = 6.9 Hz, | |
| | 3H). | |
| 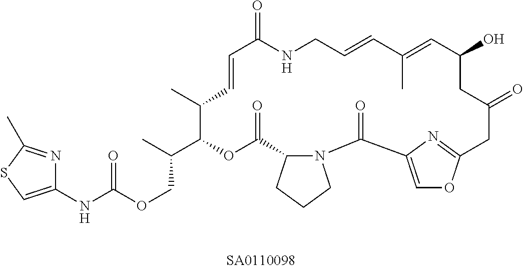 [SMILES: CC1=C\[C@@H](O)CC(=O)Cc2nc(co2)C(=O)N2CCC[C@@H]2C(=O)O[C@H]([C@@H](C)COC(=O)Nc2csc(C)n2)[C@H](C)/C=C/C(=O)NC\C=C\1] | ¹H NMR (400 MHz, CDCl₃) δ 8.81 (s, 1H), 8.66 (s, 1H), 7.08 (s, 1H), 6.47 (dd, J = 16.3, 4.9 Hz, 1H), 6.37-6.18 (m, 1H), 6.10 (d, J = 15.7 Hz, 1H), 5.78 (dd, J = 16.4, 1.8 Hz, 1H), 5.68 (ddd, J = 15.5, 9.0, 4.5 Hz, 1H), 5.44 (d, J = 8.7 Hz, 1H), 5.14 (dd, J = 5.8, 2.1 Hz, 1H), 4.91 (dt, J = 9.6, 5.6 Hz, 1H), 4.70 (dd, J = 8.8, 3.2 Hz, 1H), 4.45 (ddd, J = 14.2, 9.0, 4.7 Hz, 1H), 4.19 (dd, J = 10.9, 3.5 Hz, 1H), 4.10 (dd, J = 10.9, 4.2 Hz, 1H), 4.01 (dt, J = 11.1, 7.1 Hz, 1H), 3.87-3.71 (m, 1H), 3.81 (s, 2H), 3.36 (ddd, J = 14.8, 9.1, 3.5 Hz, 1H), 3.04 (dd, J = 17.4, 5.3 Hz, 1H), 2.91 (dd, J = 17.4, 5.3 | 684.3 |
| | Hz, 1H), 2.85 (br s, 1H), 2.76-2.67 | |
| | (m, 1H), 2.63 (s, 3H), 2.26-2.10 | |
| | (m, 2H), 2.01-1.82 (m, 3H), 1.72 | |
| | (s, 3H), 1.14 (d, J = 3.7 Hz, 3H), | |
| | 1.12 (d, J = 3.7 Hz, 3H). ¹³C NMR | |
| | (100 MHz, CDCl₃) δ 202.7, 171.5, | |
| | 166.6, 163.7, 160.2, 157.0, 153.7, | |
| | 146.8, 145.6, 143.7, 136.9, 136.7, | |
| | 134.1, 132.8, 125.2, 124.3, 98.2, | |
| | 76.5, 68.67 65.1, 59.7, 48.8, 48.5, | |
| | 43.0, 41.0, 39.4, 35.0, 28.3, 25.2, | |
| | 18.9, 13.8, 12.7, 10.7. | |
| 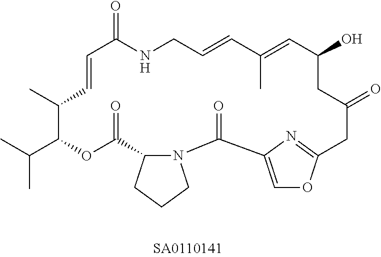 [SMILES: CC1=C\[C@@H](O)CC(=O)Cc2nc(co2)C(=O)N2CCC[C@@H]2C(=O)O[C@H](C(C)C)[C@H](C)/C=C/C(=O)NC\C=C\1] | ¹H NMR (400 MHz, CDCl₃) δ 7.91 (s, 1H), 6.53 (dd, J = 16.3, 4.7 Hz, 1H), 6.40 (dd, J = 8.4, 4.1 Hz, 1H), 6.12 (d, J = 15.6 Hz, 1H), 5.76 (dd, J = 16.2, 1.9 Hz, 1H), 5.61 (ddd, J = 15.6, 8.9, 4.4 Hz, 1H), 5.40 (d, J = 8.9 Hz, 1H), 4.91 (dt, J = 8.7, 6.1 Hz, 1H), 4.82-4.64 (m, 2H), 4.32 (ddd, J = 13.7, 8.5, 4.6 Hz, 1H), 3.98 (d, J = 2.3 Hz, 2H), 3.85 (dt, J = 10.9, 7.3 Hz, 1H), 3.71 (ddd, J = 11.1, 7.8, 4.9 Hz, 1H), 3.45 (ddd, J = 14.8, 9.0, 4.0 Hz, 1H), 3.05 (dd, J = 16.8, 6.6 Hz, 2H), 2.82 (dd, J = 16.8, 5.7 Hz, 1H), 2.73 (ddq, J = 6.9, 4.4, 2.3 Hz, 1H), 2.14 | 544.4 |
| | (dtd, J = 13.2, 9.4, 7.4 Hz, 1H), 1.99- | |
| | 1.71 (m, 4H), 1.68 (d, J = 1.2 Hz, | |
| | 3H), 1.05 (d, J = 6.8 Hz, 3H), 0.98 | |
| | (d, J = 6.5 Hz, 3H), 0.94 (d, J = 6.7 | |
| | Hz, 3H). ¹³C NMR (100 MHz, CDCl₃) | |
| | δ 203.5, 171.7, 166.4, 161.4, 160.6, | |
| | 150.6, 144.9, 136.5, 134.4, 132.7, | |
| | 126.1, 125.2, 123.9, 81.2, 64.7, | |
| | 59.6, 49.4, 49.1, 47.5, 40.9, 36.57, | |
| | 29.4, 28.5, 25.1, 19.7, 18.6, 12.7, | |
| | 10.2. | |
| 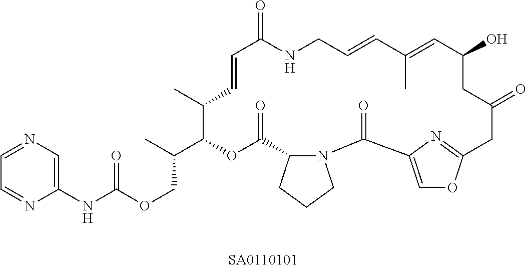 [SMILES: CC1=C\[C@@H](O)CC(=O)Cc2nc(co2)C(=O)N2CCC[C@@H]2C(=O)O[C@H]([C@@H](C)COC(=O)Nc2cnccn2)[C@H](C)/C=C/C(=O)NC\C=C\1] | ¹H NMR (400 MHz, CDCl₃) δ 9.31 (br s, 1H), 8.84 (s, 1H), 8.38 (d, J = 3.5 Hz, 1H), 8.28 (d, J = 2.6 Hz, 1H), 8.22 (t, J = 2.0 Hz, 1H), 6.49 (dd, J = 16.3, 4.9 Hz, 1H), 6.29 (d, J = 8.7 Hz, 1H), 6.10 (d, J = 15.7 Hz, 1H), 5.79 (dd, J = 16.2, 1.8 Hz, 1H), 5.68 (ddd, J = 15.6, 8.8, 4.5 Hz, 1H), 5.43 (d, J = 8.7 Hz, 1H), 5.17 (dd, J = 6.1, 2.1 Hz, 1H), 4.91 (dt, J = 8.7, 5.5 Hz, 1H), 4.71 (dd, J = 8.6, 3.3 Hz, 1H), 4.43 (dd, J = 11.7, 6.4 Hz, 1H), 4.27 (dd, J = 11.0, 3.6 Hz, 1H), 4.15 (dd, J = 11.0, 4.2 Hz, 1H), 4.07-3.96 (m, 1H), 3.89-3.83 (m, 1H), 3.81 (s, 2H), 3.39 (ddd, J = 14.9, 8.9, 3.6 Hz, 1H), 3.03 (dd, J = | 665.3 |
| | 17.1, 5.5 Hz, 1H), 2.91 (dd, J = | |
| | 17.1, 5.5 Hz, 1H), 2.80-2.70 (m, | |
| | 1H), 2.27-2.14 (m, 2H), 1.90 (ddd, | |
| | J = 18.9, 10.3, 4.4 Hz, 3H), 1.72 (s, | |
| | 3H), 1.15 (d, J = 6.5 Hz, 3H), 1.13 | |
| | (s, 3H). ¹³C NMR (100 MHz, CDCl₃) | |
| | δ 202.5, 171.6, 166.5, 160.3, 157.1, | |
| | 153.2, 148.5, 145.0, 143.6, 141.7, | |
| | 139.3, 136.9, 136.51 136.1, 134.2, | |
| | 132.8, 125.2, 124.4, 76.4, 68.9, | |
| | 65.1, 59.7, 48.8, 48.6, 43.1, 40.9, | |
| | 39.0, 34.9, 28.3, 25.2, 13.7, 12.7, | |
| | 10.8. | |
| 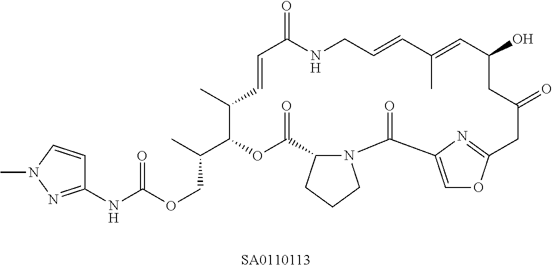 [SMILES: CC1=C\[C@@H](O)CC(=O)Cc2nc(co2)C(=O)N2CCC[C@@H]2C(=O)O[C@H]([C@@H](C)COC(=O)Nc2ccn(C)n2)[C@H](C)/C=C/C(=O)NC\C=C\1] | ¹H NMR (400 MHz, CDCl₃) δ 8.57 (s, 1H), 8.44 (s, 1H), 7.21 (d, J = 2.3 Hz, 1H), 6.47 (dd, J = 16.4, 5.0 Hz, 1H), 6.44 (br s, 1H), 6.31 (brs, 1H), 6.10 (d, J = 15.6 Hz, 1H), 5.77 (dd, J = 16.3, 1.8 Hz, 1H), 5.69 (ddd, J = 15.6, 9.0, 4.5 Hz, 1H), 5.42 (d, J = 8.6 Hz, 1H), 5.18-5.01 (m, 1H), 4.91 (q, J = 6.4 Hz, 1H), 4.71 (dd, J = 8.7, 3.2 Hz, 1H), 4.45 (ddd, J = 14.1, 8.8, 4.6 Hz, 1H), 4.13 (qd, J = 11.0, 4.0 Hz, 2H), 4.00 (dt, J = 11.3, 7.1 Hz, 1H), 3.85-3.75 (m, 1H), 3.81 (s, 2H), 3.78 (s, 3H), 3.37 (ddd, J = 14.3, 9.1, 3.5 Hz, 1H), 3.03 (dd, J = 17.3, 5.4 Hz, 1H), 2.90 (dd, J = 17.3, 5.6 Hz, 1H), 2.83 | 667.2 |
| | (br s, 1H), 2.72 (br t, J = 6.5 Hz, | |
| | 1H), 2.25-2.10 (m, 2H), 1.99- | |
| | 1.82 (m, 3H), 1.72 (d, J = 1.2 Hz, | |
| | 3H), 1.12 (d, J = 6.6 Hz, 3H). ¹³C | |
| | NMR (100 MHz, CDCl₃) δ 202.7, | |
| | 171.5, 166.5, 160.2, 157.0, 153.8, | |
| | 147.3, 145.4, 143.8, 136.9, 136.6, | |
| | 134.1, 132.7, 130.8, 125.3, 124.3, | |
| | 96.1, 76.7, 68.2, 65.1, 59.7, 48.7, | |
| | 48.5, 43.1, 41.0, 39.1, 38.7, 35.0, | |
| | 28.3, 25.1, 13.9, 12.7, 10.8. | |
| 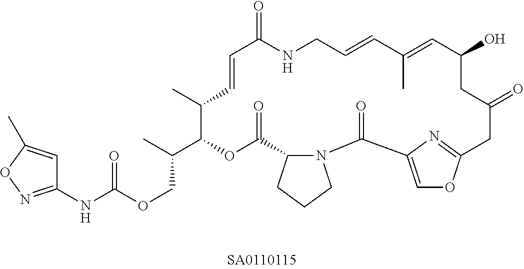 [SMILES: CC1=C\[C@@H](O)CC(=O)Cc2nc(co2)C(=O)N2CCC[C@@H]2C(=O)O[C@H]([C@@H](C)COC(=O)Nc2cc(C)on2)[C@H](C)/C=C/C(=O)NC\C=C\1] | ¹H NMR (400 MHz, CDCl₃) δ 9.35 (s, 1H), 8.85 (s, 1H), 6.52 (s, 1H), 6.45 (dd, J = 16.3, 4.7 Hz, 1H), 6.11 (d, J = 15.7 Hz, 2H), 5.78 (dd, J = 16.4, 1.8 Hz, 1H), 5.68 (ddd, J = 14.9, 9.3, 4.5 Hz, 1H), 5.49 (d, J = 8.7 Hz, 1H), 5.23 (s, 1H), 4.95- 4.85 (m, 1H), 4.70 (dd, J = 8.7, 3.3 Hz, 1H), 4.52-4.42 (m, 1H), 4.21 (dd, J = 10.8, 2.6 Hz, 1H), 4.10 (dd, J = 10.8, 3.6 Hz, 1H), 4.10-4.00 (m, 1H), 3.88-3.75 (m, 3H), 3.34 (ddd, J = 13.8, 9.3, 3.4 Hz, 1H), 3.05 (dd, J = 17.2, 4.9 Hz, 1H), 2.91 (dd, J = 17.8, 5.7 Hz, 1H), 2.78 (br s, 1H), 2.72-2.63 (m, 1H), 2.37 (d, J = 0.9 Hz, 2H), 2.23-2.12 (m, | 668.1 |
| | 2H), 1.98-1.89 (m, 1H), 1.91 | |
| | 1.79 (m, 2H), 1.73 (d, J = 1.1 Hz, | |
| | 3H), 1.18 (s, 3H), 1.15 (d, J = 6.9 | |
| | Hz, 3H). | |
| 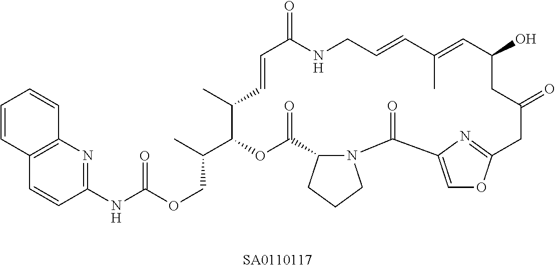 [SMILES: CC1=C\[C@@H](O)CC(=O)Cc2nc(co2)C(=O)N2CCC[C@@H]2C(=O)O[C@H]([C@@H](C)COC(=O)Nc2ccc3ccccc3n2)[C@H](C)/C=C/C(=O)NC\C=C\1] | ¹H NMR (400 MHz, CDCl₃) δ 8.61 (br s, 1H), 8.43 (s, 1H), 8.18 (q, J = 9.0 Hz, 2H), 7.84-7.72 (m, 2H), 7.65 (ddd, J = 8.5, 6.9, 1.5 Hz, 1H), 7.44 (ddd, J = 8.1, 6.9, 1.2 Hz, 1H), 6.49 (dd, J = 16.3, 4.9 Hz, 1H), 6.35 (d, J = 6.0 Hz, 1H), 6.12 (d, J = 15.7 Hz, 1H), 5.81 (dd, J = 16.3, 1.8 Hz, 1H), 5.70 (ddd, J = 15.6, 8.9, 4.5 Hz, 1H), 5.43 (d, J = 8.7 Hz, 1H), 5.13 (dd, J = 7.1, 2.1 Hz, 1H), 4.93 (dt, J = 8.7, 5.5 Hz, 1H), 4.72 (dd, J = 8.6, 3.3 Hz, 1H), 4.47 (ddd, J = 14.3, 9.1, 4.7 Hz, 1H), 4.25 (dd, J = 11.0, 4.1 Hz, 1H), 4.15 (dd, J = 11.0, 4.6 Hz, 1H), 4.00 (dt, J = 11.3, 7.2 Hz, 1H), 3.87-3.75 (m, 1H), | 714.2 |
| | 3.82 (s, 3H), 3.38 (ddd, J = 14.8, | |
| | 9.0, 3.6 Hz, 1H), 3.06 (dd, J = 17.3, | |
| | 5.5 Hz, 1H), 2.90 (dd, J = 17.2, 5.5 | |
| | Hz, 1H), 2.80-2.70 (m, 1H), 2.30- | |
| | 2.11 (m, 2H), 2.02-1.78 (m, 3H), | |
| | 1.73 (d, J = 1.2 Hz, 3H), 1.14 (d, J = | |
| | 3.2 Hz, 3H), 1.13 (d, J = 3.2 Hz, | |
| | 3H). ¹³C NMR (100 MHz, CDCl₃) δ | |
| | 202.6, 171.6, 166.5, 160.2, 157.0, | |
| | 153.6, 151.0, 146.5, 144.9, 143.7, | |
| | 138.7, 137.0, 136.6, 134.2, 132.7, | |
| | 130.1, 127.6, 126.9, 125.9, 125.3, | |
| | 125.0 124.45 113.1, 76.7, 68.0, | |
| | 65.1, 59.7, 48.7, 48.5, 43.1, 41.0, | |
| | 38.4, 34.8, 28.3, 25.2, 14.0, 12.8, | |
| | 10.8. | |
| 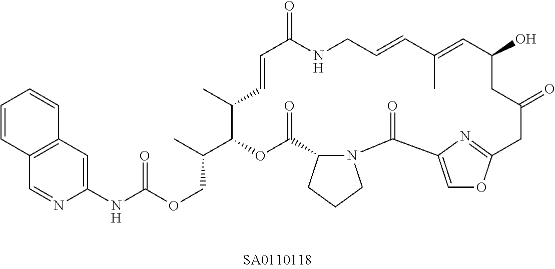 [SMILES: CC1=C\[C@@H](O)CC(=O)Cc2nc(co2)C(=O)N2CCC[C@@H]2C(=O)O[C@H]([C@@H](C)COC(=O)Nc2cc3ccccc3cn2)[C@H](C)/C=C/C(=O)NC\C=C\1] | ¹H NMR (400 MHz, CDCl₃) δ 8.97 (s, 1H), 8.46 (s, 1H), 8.32 (s, 1H), 8.25 (s, 1H), 7.88 (d, J = 8.2 Hz, 1H), 7.79 (d, J = 8.3 Hz, 1H), 7.62 (ddd, J = 8.2, 6.8, 1.2 Hz, 1H), 7.45 (ddd, J = 8.1, 6.8, 1.1 Hz, 1H), 6.50 (dd, J = 16.3, 4.9 Hz, 1H), 6.36 (dd, J = 8.8, 3.7 Hz, 1H), 6.10 (d, J = 15.6 Hz, 1H), 5.80 (dd, J = 16.3, 1.8 Hz, 1H), 5.69 (ddd, J = 15.6, 8.9, 4.5 Hz, 1H), 5.41 (d, J = 8.7 Hz, 1H), 5.14 (dd, J = 7.4, 2.2 Hz, 1H), 4.92 (dt, J = 8.8, 5.5 Hz, 1H), 4.72 (dd, J = 8.7, 3.3 Hz, 1H), 4.26 (dd, J = 11.0, 4.1 Hz, 1H), 4.17 (dd, J = 11.0, 4.8 Hz, 1H), 3.99 (dt, J = 11.3, 7.1 Hz, 1H), 3.81 (s, 3H), 3.37 (ddd, | 714.2 |
| | J = 14.9, 8.9, 3.6 Hz, 1H), 3.05 (dd, | |
| | J = 17.1, 5.7 Hz, 1H), 2.89 (dd, J = | |
| | 17.1, 5.5 Hz, 1H), 2.80 (ddt, J = 7.3, | |
| | 4.9, 2.2 Hz, 1H), 2.37-2.08 (m, | |
| | 2H), 2.00-1.74 (m, 4H), 1.72 (d, J = | |
| | 1.2 Hz, 3H), 1.19-1.14 (m, 3H), | |
| | 1.13 (s, 3H). | |
| | ¹³C NMR (100 MHz, CDCl₃) δ | |
| | 202.5, 171.6, 166.5, 160.3, 157.0, | |
| | 153.4, 151.0, 146.5, 144.67, 143.7, | |
| | 138.0, 137.0, 136.6, 134.2, 132.7, | |
| | 130.8, 127.5, 126.6, 126.1, 125.5, | |
| | 125.3, 124.5, 106.3, 77.0, 67.6, | |
| | 65.1, 59.7, 48.7, 48.5, 43.2, 40.9, | |
| | 38.2, 34.7, 28.4, 25.1, 14.1, 12.7, | |
| | 10.9. | |
| 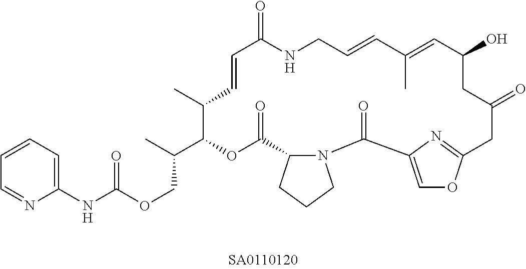 [SMILES: CC1=C\[C@@H](O)CC(=O)Cc2nc(co2)C(=O)N2CCC[C@@H]2C(=O)O[C@H]([C@@H](C)COC(=O)Nc2ccccn2)[C@H](C)/C=C/C(=O)NC\C=C\1] | ¹H NMR (400 MHz, CDCl₃) δ 8.52 (s, 1H), 8.32 (s, 1H), 8.29- 8.21 (m, 1H), 7.95 (d, J = 8.4 Hz, 1H), 7.68 (ddd, J = 8.7, 7.4, 1.9 Hz, 1H), 6.99 (ddd, J = 7.3, 4.9, 1.0 Hz, 1H), 6.55-6.44 (m, 1H), 6.37 (dd, J = 8.9, 3.6 Hz, 1H), 6.11 (d, J = 15.6 Hz, 1H), 5.79 (dd, J = 16.3, 1.8 Hz, 1H), 5.69 (ddd, J = 15.6, 8.9, 4.6 Hz, 1H), 5.41 (d, J = 8.7 Hz, 1H), 5.11 (dd, J = 7.3, 2.2 Hz, 1H), 4.92 (dt, J = 8.7, 5.6 Hz, 1H), 4.71 (dd, J = 8.7, 3.3 Hz, 1H), 4.45 (ddd, J = 14.1, 8.9, 4.6 Hz, 1H), 4.21 (dd, J = 11.1, 4.1 Hz, 1H), 4.12 (dd, J = 11.1, 4.7 Hz, 1H), 4.05-3.97 (m, 1H), 3.84-3.74 (m, 1H), 3.81 (s, | 664.3 |
| | 2H), 3.39 (ddd, J = 14.9, 8.9, 3.6 | |
| | Hz, 1H), 3.05 (dd, J = 17.2, 5.7 Hz, | |
| | 1H), 2.89 (dd, J = 17.3, 5.3 Hz, 1H), | |
| | 2.82-2.70 (m, 1H), 2.27-2.11 (m, | |
| | 2H), 1.98-1.78 (m, 3H), 1.72 (d, J = | |
| | 1.2 Hz, 3H), 1.12 (d, J = 6.8 Hz, | |
| | 6H). ¹³C NMR (100 MHz, CDCl₃) δ | |
| | 202.5, 171.6, 166.5, 160.3, 157.0, | |
| | 153.4, 151.6, 147.6, 144.7, 143.7, | |
| | 138.4, 136.9, 136.6, 134.2, 132.7, | |
| | 125.3, 124.4, 119.0, 112.6, 76.9, | |
| | 67.7, 65.1, 59.7, 48.7, 48.5, 43.2, | |
| | 40.9, 38.2, 34.7, 28.3, 25.1, 14.03, | |
| | 12.7, 10.9. | |
| 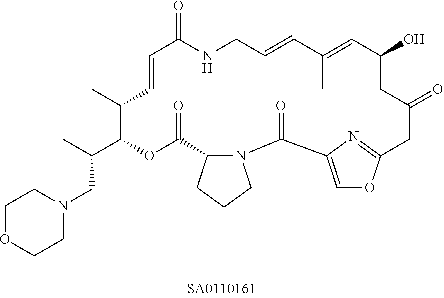 [SMILES: CC1=C\[C@@H](O)CC(=O)Cc2nc(co2)C(=O)N2CCC[C@@H]2C(=O)O[C@H]([C@@H](C)CN2CCOCC2)[C@H](C)/C=C/C(=O)NC\C=C\1] | ¹H NMR (400 MHz, CDCl₃) δ 6.50 (dd, J = 16.3, 4.1 Hz, 1H), 6.20- 6.05 (m, 2H), 5.77 (dd, J = 16.3, 2.0 Hz, 1H), 5.56 (ddd, J = 14.8, 9.5, 4.1 Hz, 1H), 5.41 (d, J = 8.9 Hz, 1H), 5.05-4.95 (m, 2H), 4.80 (dd, J = 8.7, 3.3 Hz, 1H), 4.49 (ddd, J = 14.1, 9.0, 4.1 Hz, 1H), 3.88 (d, J = 17.2 Hz, 1H), 3.81-3.56 (m, 7H), 3.40 (ddd, J = 13.5, 9.7, 3.1 Hz, 1H), 2.91 (dd, J = 15.8, 5.8 Hz, 1H), 2.95-2.80 (m, 1H), 2.73 (dd, J = 15.7, 5.8 Hz, 1H), 2.52-2.29 (m, 6H), 2.29-1.75 (m, 5H), 1.65 (s, 3H), 1.11 (d, J = 6.9 Hz, 3H), 0.98 (d, J = 6.5 Hz, 3H), 0.85 (s, 9H), 0.30 (s, 9H), 0.05 (s, 3H), 0.02 (s, 3H). ¹³C NMR (100 MHz, CDCl₃) δ 201.0, 171.9, 166.4, 161.8, 161.3, | 613.3 |
| | 159.6, 145.0, 144.8, 136.7, 134.7, | |
| | 132.5, 124.9, 123.6, 79.1, 67.0, | |
| | 65.4, 62.6, 58.7, 54.1, 50.6, 48.4, | |
| | 43.7, 41.3, 37.6, 31.7, 28.3, 25.7, | |
| | 24.8, 18.1, 15.8, 12.7, 10.9, −1.9, −4.5, | |
| | −5.0. | |
| 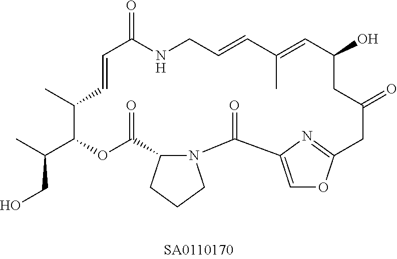 [SMILES: CC1=C\[C@@H](O)CC(=O)Cc2nc(co2)C(=O)N2CCC[C@@H]2C(=O)O[C@H]([C@H](C)CO)[C@H](C)/C=C/C(=O)NC\C=C\1] | ¹H NMR (300 MHz, CDCl₃) δ 8.09 (s, 1H), 6.47 (dd, J = 16.4, 4.9 Hz, 1H), 6.33 (d, J = 8.9 Hz, 1H), 6.12 (d, J = 15.7 Hz, 1H), 5.81- 5.71 (m, 1H), 5.74-5.60 (m, 1H), 5.42 (d, J = 8.7 Hz, 1H), 5.02 (dd, J = 10.7, 1.8 Hz, 1H), 4.89 (d, J = 6.1 Hz, 1H), 4.59 (ddd, J = 8.5, 4.2 Hz, 1H), 4.49 (ddd, J = 14.2, 9.2, 4.8 Hz, 1H), 4.03 (dt, J = 12.6, 6.7 Hz, 1H), 3.81 (m, 4H), 3.61-3.45 (m, 1H), 3.42-3.25 (m, 1H), 3.02 (dd, J = 17.4, 5.4 Hz, 1H), 2.88 (dd, J = 17.4, 5.3 Hz, 1H), 2.77-2.67 (m, 2H), 2.64 (br s, 1H), 2.25-2.12 (m, 1H), 2.00-1.75 (m, 4H), 1.72 (s, | 544.3 |
| | 3H), 1.05 (d, J = 6.9 Hz, 6H). | |
| 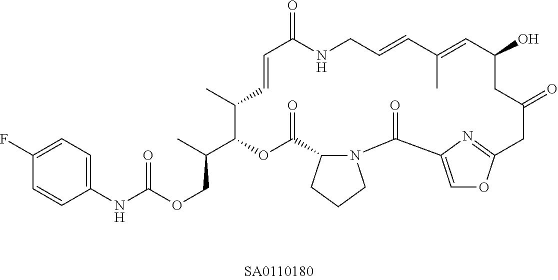 [SMILES: CC1=C\[C@@H](O)CC(=O)Cc2nc(co2)C(=O)N2CCC[C@@H]2C(=O)O[C@H]([C@H](C)COC(=O)Nc2ccc(F)cc2)[C@H](C)/C=C/C(=O)NC\C=C\1] | ¹H NMR (400 MHz, CDCl₃) δ 9.07 (br s, 1H), 8.19 (s, 1H), 7.50- 7.41 (m, 2H), 6.94 (t, J = 8.7 Hz, 2H), 6.53 (dd, J = 16.2, 4.2 Hz, 1H), 6.17 (d, J = 15.6 Hz, 1H), 5.99- 5.91 (m, 1H), 5.80 (dd, J = 16.2, 2.0 Hz, 1H), 5.69 (ddd, J = 15.7, 9.3, 4.5 Hz, 1H), 5.56 (d, J = 8.8 Hz, 1H), 5.16 (dd, J = 9.6, 1.9 Hz, 1H), 5.03-4.90 (m, 1H), 4.75 (dd, J = 8.7, 2.6 Hz, 1H), 4.58 (dd, J = 11.6, 3.7 Hz, 1H), 4.36 (td, J = 8.6, 8.2, 4.0 Hz, 1H), 3.98-3.85 (m, 2H), 3.84 (s, 2H), 3.76-3.68 (m, 1H), 3.48 (ddd, J = 14.1, 9.3, 4.2 Hz, 1H), 3.12 (dd, J = 17.7, 4.9 Hz, 1H), 2.91 (dd, J = 17.7, 6.1 Hz, 1H), 2.80- 2.60 (m, 2H), 2.40-2.25 (m, 1H), | 681.2 |
| | 2.20-2.03 (m, 2H), 2.00-1.80 (m, | |
| | 2H), 1.74 (d, J = 1.2 Hz, 3H), 1.11 | |
| | (d, J = 6.8 Hz, 3H), 0.96 (d, J = 7.1 | |
| | Hz, 3H). | |
| 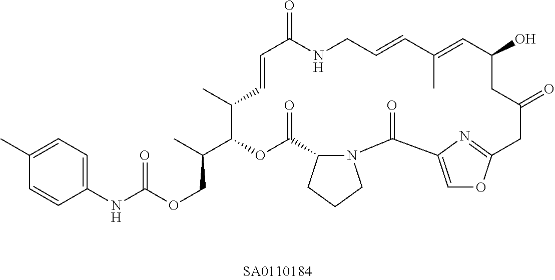 [SMILES: CC1=C\[C@@H](O)CC(=O)Cc2nc(co2)C(=O)N2CCC[C@@H]2C(=O)O[C@H]([C@H](C)COC(=O)Nc2ccc(C)cc2)[C@H](C)/C=C/C(=O)NC\C=C\1] | ¹H NMR (400 MHz, CDCl₃) δ 8.91 (br s, 1H), 8.18 (s, 1H), 7.36 (d, J = 8.5 Hz, 2H), 7.05 (d, J = 8.2 Hz, 2H), 6.53 (dd, J = 16.2, 4.2 Hz, 1H), 6.17 (d, J = 15.6 Hz, 1H), 6.04 (d, J = 7.8 Hz, 1H), 5.79 (dd, J = 16.2, 2.0 Hz, 1H), 5.67 (ddd, J = 14.9, 9.3, 4.4 Hz, 1H), 5.55 (d, J = 8.7 Hz, 1H), 5.15 (dd, J = 9.7, 1.9 Hz, 1H), 4.95 (dt, J = 9.0, 5.6 Hz, 1H), 4.75 (dd, J = 8.7, 2.7 Hz, 1H), 4.54 (dd, J = 11.5, 3.8 Hz, 1H), 4.36 (ddd, J = 13.5, 8.2, 4.5 Hz, 1H), 4.01-3.84 (m, 2H), 3.83 (s, 2H), 3.74 (dd, J = 11.5, 9.3 Hz, 1H), 3.48 (ddd, J = 14.2, 9.3, 4.2 Hz, 1H), 3.12 (dd, J = 17.6, 5.0 Hz, 1H), 2.91 (dd, J = 17.6, 6.1 Hz, 1H), 2.83 (br s, 1H), | 677.1 |
| | 2.77-2.67 (m, 1H), 2.40-2.31 (m, | |
| | 1H), 2.27 (s, 3H), 2.15-2.00 (m, | |
| | 1H), 1.99-1.80 (m, 3H), 1.73 (d, J = | |
| | 1.2 Hz, 3H), 1.10 (d, J = 6.8 Hz, | |
| | 3H), 0.95 (d, J = 7.0 Hz, 3H). | |
| 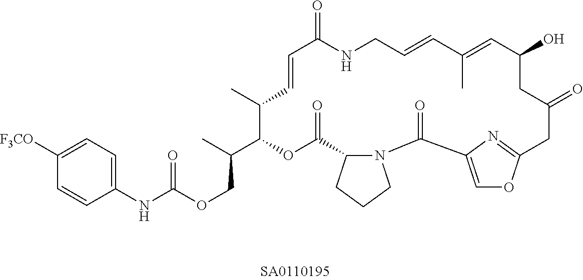 [SMILES: CC1=C\[C@@H](O)CC(=O)Cc2nc(co2)C(=O)N2CCC[C@@H]2C(=O)O[C@H]([C@H](C)COC(=O)Nc2ccc(OC(F)(F)F)cc2)[C@H](C)/C=C/C(=O)NC\C=C\1] | ¹H NMR (400 MHz, CDCl₃) δ 9.25 (br s, 1H), 8.19 (s, 1H), 7.52 (d, J = 9.1 Hz, 2H), 7.10 (d, J = 8.6 Hz, 2H), 6.54 (dd, J = 16.2, 4.2 Hz, 1H), 6.18 (d, J = 15.6 Hz, 1H), 5.96 (dd, J = 8.2, 4.3 Hz, 1H), 5.80 (dd, J = 16.2, 2.0 Hz, 1H), 5.68 (ddd, J = 15.6, 9.3, 4.4 Hz, 1H), 5.57 (d, J = 8.6 Hz, 1H), 5.17 (dd, J = 9.6, 1.9 Hz, 1H), 4.96 (q, J = 7.0, 6.5 Hz, 1H), 4.74 (dd, J = 8.8, 2.6 Hz, 1H), 4.61 (dd, J = 11.5, 3.5 Hz, 1H), 4.36 (ddd, J = 13.7, 8.3, 4.5 Hz, 1H), 4.02-3.85 (m, 2H), 3.84 (s, 2H), 3.72 (dd, J = 11.6, 9.8 Hz, 1H), 3.50 (ddd, J = 14.2, 9.2, 4.3 Hz, 1H), 3.13 (dd, J = 17.7, 4.9 Hz, 1H), 2.91 (dd, J = 17.7, 6.2 Hz, 1H), 2.82- | 747.1 |
| | 2.62 (m, 2H), 2.40-2.25 (m, 1H), | |
| | 2.16-2.04 (m, 1H), 2.02-1.81 (m, | |
| | 3H), 1.74 (d, J = 1.2 Hz, 3H), 1.11 | |
| | (d, J = 6.8 Hz, 3H), 0.96 (d, J = 7.0 | |
| | Hz, 3H). ¹³C NMR (100 MHz, CDCl₃) | |
| | δ 202.6, 170.6, 165.8, 160.3, 157.5, | |
| | 153.7, 151.2, 144.5, 144.5, 137.9, | |
| | 137.0, 136.7, 134.1, 132.9, 125.1, | |
| | 124.2, 121.6, 119.3, 79.3, 69.1, | |
| | 64.9, 59.9, 49.2, 48.7, 42.6, 41.1, | |
| | 37.1, 33.8, 28.5, 24.7, 13.2, 12.7, | |
| | 10.2. | |
| 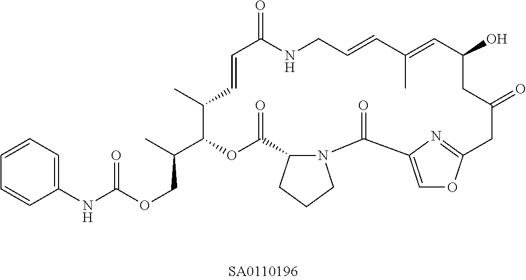 [SMILES: CC1=C\[C@@H](O)CC(=O)Cc2nc(co2)C(=O)N2CCC[C@@H]2C(=O)O[C@H]([C@H](C)COC(=O)Nc2ccccc2)[C@H](C)/C=C/C(=O)NC\C=C\1] | ¹H NMR (400 MHz, CDCl₃) δ 9.04 (br s, 1H), 8.20 (s, 1H), 7.49 (d, J = 8.0 Hz, 2H), 7.32-7.20 (m, 2H), 7.00 (t, J = 7.4 Hz, 1H), 6.54 (dd, J = 16.2, 4.2 Hz, 1H), 6.18 (d, J = 15.6 Hz, 1H), 6.00 (dd, J = 8.4, 4.3 Hz, 1H), 5.80 (dd, J = 16.2, 2.0 Hz, 1H), 5.69 (ddd, J = 15.6, 9.3, 4.4 Hz, 1H), 5.57 (d, J = 8.7 Hz, 1H), 5.17 (dd, J = 9.7, 1.9 Hz, 1H), 4.96 (dt, J = 9.0, 5.6 Hz, 1H), 4.77 (dd, J = 8.7, 2.7 Hz, 1H), 4.58 (dd, J = 11.6, 3.7 Hz, 1H), 4.37 (ddd, J = 13.6, 8.3, 4.4 Hz, 1H), 4.00-3.80 (m, 2H), 3.84 (s, 2H), 3.75 (dd, J = 11.6, 9.5 Hz, 1H), 3.48 (ddd, J = 14.0, 9.3, 4.2 Hz, 1H), 3.13 (dd, J = 17.7, 4.9 Hz, 1H), 2.92 (dd, J = | 663.1 |
| | 17.7, 6.1 Hz, 1H), 2.78 (br s, 1H), | |
| | 2.76-2.66 (m, 1H), 2.40-2.25 (m, | |
| | 1H), 2.17-2.03 (m, 1H), 2.00- | |
| | 1.80 (m, 2H), 1.74 (d, J = 1.1 Hz, | |
| | 3H), 1.11 (d, J = 6.9 Hz, 3H), 0.97 | |
| | (d, J = 7.0 Hz, 3H). ¹³C NMR (100 | |
| | MHz, CDCl₃) δ 202.6, 170.8, 165.9, | |
| | 160.2, 157.3, 153.7, 144.5, 139.1, | |
| | 137.1, 136.7, 134.1, 132.9, 128.8, | |
| | 125.1, 124.2, 122.7, 118.4, 79.2, | |
| | 68.4, 64.9, 59.9, 49.2, 48.7, 42.6, | |
| | 41.1, 37.1, 33.8, 28.5, 24.7, 13., | |
| | 12.74, 10.2. | |
| 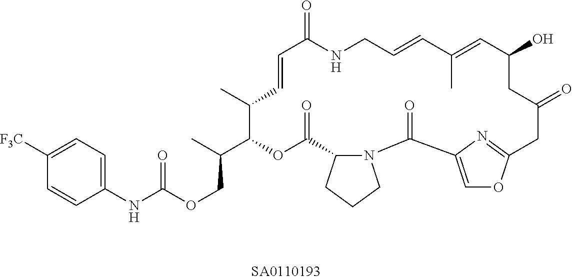 [SMILES: CC1=C\[C@@H](O)CC(=O)Cc2nc(co2)C(=O)N2CCC[C@@H]2C(=O)O[C@H]([C@H](C)COC(=O)Nc2ccc(C(F)(F)F)cc2)[C@H](C)/C=C/C(=O)NC\C=C\1] | ¹H NMR (400 MHz, CDCl₃) δ 9.40 (s, 1H), 8.20 (s, 1H), 7.62 (d, J = 8.5 Hz, 2H), 7.49 (d, J = 8.6 Hz, 2H), 6.54 (dd, J = 16.2, 4.2 Hz, 1H), 6.18 (d, J = 15.6 Hz, 1H), 5.98 (dd, J = 8.3, 4.4 Hz, 1H), 5.80 (dd, J = 16.2, 2.0 Hz, 1H), 5.68 (ddd, J = 15.6, 9.3, 4.4 Hz, 1H), 5.57 (d, J = 8.6 Hz, 1H), 5.18 (dd, J = 9.6, 2.0 Hz, 1H), 4.96 (dt, J = 9.3, 5.6 Hz, 1H), 4.73 (dd, J = 8.7, 2.6 Hz, 1H), 4.62 (dd, J = 11.6, 3.6 Hz, 1H), 4.35 (ddd, J = 13.7, 8.2, 4.5 Hz, 1H), 3.98-3.86 (m, 2H), 3.84 (s, 2H), 3.73 (dd, J = 11.6, 9.8 Hz, 1H), 3.50 (ddd, J = 14.2, 9.3, 4.3 Hz, 1H), 3.13 (dd, J = 17.7, 4.9 Hz, 1H), 2.92 (dd, J = 17.7, 6.1 Hz, 1H), 2.80 (br | 731.3 |
| | s, 1H), 2.80-2.65 (m, 1H), 2.40- | |
| | 2.25 (m, 1H), 2.18-2.05 (m, 1H), | |
| | 2.00-1.80 (m, 3H), 1.74 (d, J = 1.2 | |
| | Hz, 3H), 1.11 (d, J = 6.9 Hz, 3H), | |
| | 0.97 (d, J = 7.0 Hz, 3H). ¹³C NMR | |
| | (100 MHz, CDCl₃) δ 202.6, 170.6, | |
| | 165.7, 160.3, 157.5, 153.5, 144.6, | |
| | 144.4, 142.3, 137.0, 136.7, 134.1, | |
| | 133.0, 126.0 (q, ³J = 4.2 Hz), 125.1, | |
| | 124.2, 117.9, 79.3, 69.2, 64.9, 59.9, | |
| | 49.2, 48.7, 42.6, 41.1, 37.1, 33.7, | |
| | 28.5, 24.7, 13.2, 12.7, 10.2. | |
| 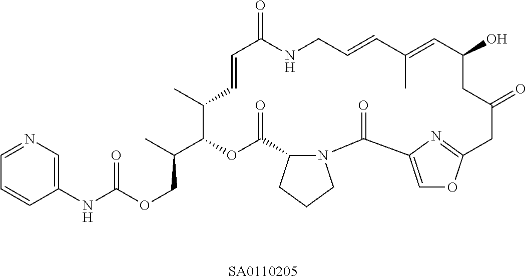 [SMILES: CC1=C\[C@@H](O)CC(=O)Cc2nc(co2)C(=O)N2CCC[C@@H]2C(=O)O[C@H]([C@H](C)COC(=O)Nc2cccnc2)[C@H](C)/C=C/C(=O)NC\C=C\1] | ¹H NMR (400 MHz, CDCl₃) δ 9.38 (br s, 1H), 8.54 (d, J = 2.6 Hz, 1H), 8.22 (dd, J = 4.7, 1.5 Hz, 1H), 8.20 (s, 1H), 8.11 (ddd, J = 8.4, 2.6, 1.5 Hz, 1H), 7.21 (dd, J = 8.4, 4.7 Hz, 1H), 6.54 (dd, J = 16.2, 4.1 Hz, 1H), 6.18 (d, J = 15.6 Hz, 1H), 6.06 (dd, J = 8.3, 4.4 Hz, 1H), 5.80 (dd, J = 16.2, 2.0 Hz, 1H), 5.67 (ddd, J = 15.6, 9.2, 4.4 Hz, 1H), 5.57 (d, J = 8.7 Hz, 1H), 5.17 (dd, J = 9.5, 1.9 Hz, 1H), 4.95 (dt, J = 8.7, 5.6 Hz, 1H), 4.74 (dd, J = 8.7, 2.7 Hz, 1H), 4.61 (dd, J = 11.6, 3.7 Hz, 1H), 4.34 (ddd, J = 13.6, 8.2, 4.4 Hz, 1H), 3.90 (dd, J = 8.5, 5.6 Hz, 2H), 3.83 (d, J = 1.5 Hz, 2H), 3.74 (dd, J = 11.6, 9.7 Hz, 1H), 3.50 (td, J = 9.6, | 664.2 |
| | 4.6 Hz, 1H), 3.12 (dd, J = 17.5, 5.1 | |
| | Hz, 1H), 2.93 (dd, J = 17.5, 6.0 Hz, | |
| | 1H), 2.80-2.65 (m, 1H), 2.40- | |
| | 2.25 (m, 1H), 2.20-2.00 (m, 1H), | |
| | 2.00-1.80 m, 3H), 1.73 (d, J = 1.2 | |
| | Hz, 3H), 1.11 (d, J = 6.9 Hz, 3H), | |
| | 0.97 (d, J = 7.0 Hz, 3H). ¹³C NMR | |
| | (100 MHz, CDCl₃) δ 202.5, 170.6, | |
| | 165.8, 160.4, 157.5, 153.8, 144.5, | |
| | 144.5, 143.6, 140.3, 136.9, 136.6, | |
| | 136.1, 134.0, 133.1 125.3, 125.0, | |
| | 124.2, 123.5, 79.2, 69.2, 64.8, 59.8, | |
| | 49.2, 48.8, 42.6, 41.1, 37.0, 33.7, | |
| | 28.5, 24.7, 13.3, 12.7, 10.2. | |
| 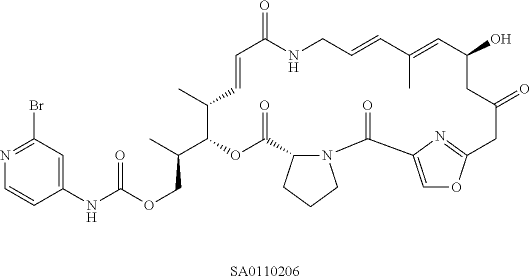 [SMILES: CC1=C\[C@@H](O)CC(=O)Cc2nc(co2)C(=O)N2CCC[C@@H]2C(=O)O[C@H]([C@H](C)COC(=O)Nc2ccnc(Br)c2)[C@H](C)/C=C/C(=O)NC\C=C\1] | ¹H NMR (400 MHz, CDCl₃) δ 9.65 (br s, 1H), 8.22 (s, 1H), 8.12 (d, J = 5.6 Hz, 1H), 7.73 (d, J = 1.9 Hz, 1H), 7.43 (dd, J = 5.7, 1.9 Hz, 1H), 6.54 (dd, J = 16.2, 4.1 Hz, 1H), 6.18 (d, J = 15.6 Hz, 1H), 6.01 (dd, J = 8.1, 4.5 Hz, 1H), 5.80 (dd, J = 16.2, 2.0 Hz, 1H), 5.67 (ddd, J = 15.6, 9.1, 4.4 Hz, 1H), 5.58 (d, J = 8.7 Hz, 1H), 5.17 (dd, J = 9.4, 1.9 Hz, 1H), 4.96 (dt, J = 8.8, 5.6 Hz, 1H), 4.70 (dd, J = 8.8, 2.6 Hz, 1H), 4.61 (dd, J = 11.6, 3.6 Hz, 1H), 4.33 (ddd, J = 13.6, 8.0, 4.4 Hz, 1H), 3.91 (dd, J = 8.6, 5.5 Hz, 2H), 3.84 (s, 2H), 3.73 (dd, J = 11.6, 9.9 Hz, 1H), 3.52 (ddd, J = 14.2, 9.2, 4.4 Hz, 1H), 3.14 (dd, J = 17.6, 5.0 Hz, 1H), 3.00- | 742.3 |
| | 2.80, (m, 1H), 2.93 (dd, J = 17.6, | |
| | 6.1 Hz, 1H), 2.78-2.66 (m, 1H), | |
| | 2.40-2.20 (m, 1H), 2.20-2.00 (m, | |
| | 1H), 1.99-1.81 (m, 3H), 1.74 (d, J = | |
| | 1.1 Hz, 3H), 1.12 (d, J = 6.8 Hz, | |
| | 3H), 0.97 (d, J = 7.0 Hz, 3H). ¹³C | |
| | NMR (100 MHz, CDCl₃) δ 202.4, | |
| | 170.4, 165.6, 160.3, 157.7, 153.0, | |
| | 150.2, 148.2, 144.6, 144.4, 142.5, | |
| | 136.9, 136.6, 134.13 133.0, 125.01 | |
| | 124.2, 116.2, 112.0, 79.2, 69.5, | |
| | 64.8, 59.8, 49.3, 48.8, 42.6, 41.1, | |
| | 37.0, 33.7, 28.4, 24.6, 13.3, 12.7, | |
| | 10.3. | |
| 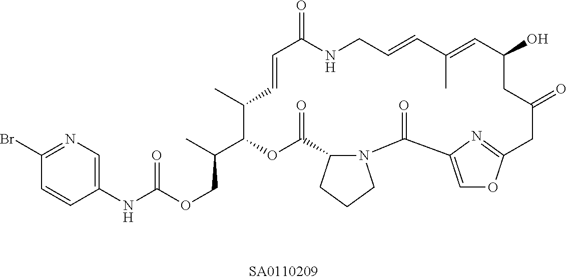 [SMILES: CC1=C\[C@@H](O)CC(=O)Cc2nc(co2)C(=O)N2CCC[C@@H]2C(=O)O[C@H]([C@H](C)COC(=O)Nc2ccc(Br)nc2)[C@H](C)/C=C/C(=O)NC\C=C\1] | ¹H NMR (400 MHz, CDCl₃) δ 9.50 (br s, 1H), 8.37 (d, J = 2.8 Hz, 1H), 8.19 (s, 1H), 8.02 (dd, J = 8.7, 2.9 Hz, 1H), 7.36 (d, J = 8.7 Hz, 1H), 6.54 (dd, J = 16.2, 4.1 Hz, 1H), 6.18 (d, J = 15.6 Hz, 1H), 5.93 (dd, J = 8.2, 4.4 Hz, 1H), 5.80 (dd, J = 16.2, 2.0 Hz, 1H), 5.68 (ddd, J = 15.6, 9.2, 4.4 Hz, 1H), 5.57 (d, J = 8.7 Hz, 1H), 5.17 (dd, J = 9.5, 1.9 Hz, 1H), 4.95 (dt, J = 8.7, 5.5 Hz, 1H), 4.71 (dd, J = 8.8, 2.6 Hz, 4.63 (dd, J = 11.6, 3.7 Hz, 1H), 4.35 (ddd, J = 13.5, 8.1, 4.6 Hz, 1H), 3.95-3.85 (m, 2H), 3.84 (d, J = 2.6 Hz, 2H), 3.71 (dd, J = 11.6, 10.0 Hz, 1H), 3.51 (ddd, J = 14.2, 9.2, 4.4 Hz, 1H), 3.13 (dd, J = 17.7, 5.0 Hz, | 742.3 |
| | 1H), 2.93 (dd, J = 17.7, 6.0 Hz, 1H), | |
| | 2.83-2.68 (m, 2H), 2.40-2.22 (m, | |
| | 1H), 2.16-2.04 (m, 1H), 1.98- | |
| | 1.83 (m, 2H), 1.74 (d, J = 1.3 Hz, | |
| | 3H), 1.72-1.62 (m, 1H), 1.12 (d, J = | |
| | 6.9 Hz, 3H), 0.97 (d, J = 7.0 Hz, | |
| | 3H). ¹³C NMR (100 MHz, CDCl₃) δ | |
| | 202.6, 170.5, 165.8, 160.4, 157.5, | |
| | 153.6, 144.6, 144.5, 140.4, 136.9, | |
| | 136.6, 135.8, 134.1, 134.0, 133.0, | |
| | 128.1, 127.7, 125.1, 124.2, 79.3, | |
| | 69.4, 64.9, 59.9, 49.2, 48.8, 42.6, | |
| | 41.1, 37.1, 33.7, 28.5, 24.7, 13.3, | |
| | 128.8, 10.2. | |
| 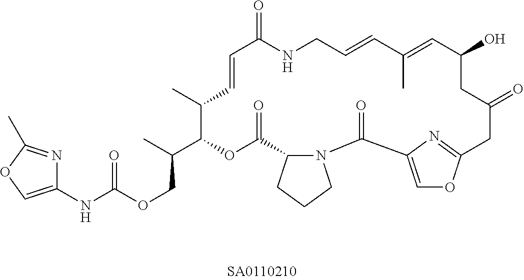 [SMILES: CC1=C\[C@@H](O)CC(=O)Cc2nc(co2)C(=O)N2CCC[C@@H]2C(=O)O[C@H]([C@H](C)COC(=O)Nc2coc(C)n2)[C@H](C)/C=C/C(=O)NC\C=C\1] | ¹H NMR (400 MHz, CDCl₃) δ 9.15 (br s, 1H), 8.53 (s, 1H), 7.67 (s, 1H), 6.49 (dd, J = 16.3, 4.3 Hz, 1H), 6.15 (d, J = 15.6 Hz, 1H), 6.04 (d, J = 9.4 Hz, 1H), 5.79 (dd, J = 16.3, 2.0 Hz, 1H), 5.68 (ddd, J = 14.9, 9.2, 4.4 Hz, 1H), 5.54 (d, J = 8.7 Hz, 1H), 5.10 (d, J = 9.4 Hz, 1H), 4.93 (q, J = 6.4 Hz, 1H), 4.76 (dd, J = 9.1, 2.8 Hz, 1H), 4.49-4.29 (m, 2H), 4.06-3.70 (m, 5H), 3.42 (ddd, J = 14.1, 9.3, 3.8 Hz, 1H), 3.09 (dd, J = 17.7, 4.9 Hz, 1H), 2.91 (dd, J = 17.8, 6.0 Hz, 1H), 2.87 (br s, 1H), 2.77-2.66 (m, 1H), 2.37 (s, 3H), 2.34-2.24 (m, 1H), 2.20-2.06 (m, 1H), 1.95-1.85 (m, 3H), 1.73 (s, 3H), 1.10 (d, J = 6.8 Hz, 3H), 0.99 | 668.2 |
| | (d, J = 7.0 Hz, 3H). ¹³C NMR (100 | |
| | MHz, CDCl₃) δ 202.8, 171.2, 166.1, | |
| | 160.3, 159.2, 157.12 153.5, 145.53 | |
| | 144.3, 137.5, 137.0, 136.7, 134.0, | |
| | 133.0, 125.1, 124.2, 123.9, 78.5, | |
| | 69.0, 64.9, 59.8, 49.0, 48.5, 42.7, | |
| | 41.1, 36.9, 33.9, 28.3, 24.9, 14.0, | |
| | 13.7, 12.7, 10.2. | |
| 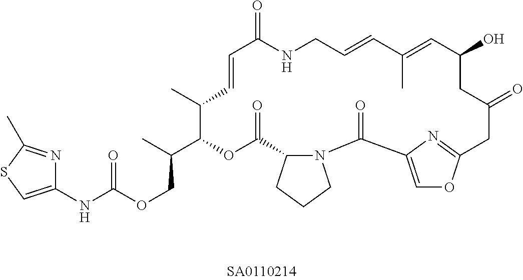 [SMILES: CC1=C\[C@@H](O)CC(=O)Cc2nc(co2)C(=O)N2CCC[C@@H]2C(=O)O[C@H]([C@H](C)COC(=O)Nc2csc(C)n2)[C@H](C)/C=C/C(=O)NC\C=C\1] | ¹H NMR (400 MHz, CDCl₃) δ 9.32 (br s, 1H), 8.55 (s, 1H), 7.07 (s, 1H), 6.48 (dd, J = 16.3, 4.4 Hz, 1H), 6.14 (d, J = 15.6 Hz, 1H), 6.07 (d, J = 8.3 Hz, 1H), 5.79 (dd, J = 16.3, 2.0 Hz, 1H), 5.68 (ddd, J = 15.5, 9.3, 4.4 Hz, 1H), 5.52 (d, J = 8.7 Hz, 1H), 5.07 (dd, J = 9.8, 1.8 Hz, 1H), 4.92 (dt, J = 9.1, 5.4 Hz, 1H), 4.74 (dd, J = 8.7, 3.1 Hz, 1H), 4.44 (ddd, J = 13.8, 8.8, 4.5 Hz, 1H), 4.32 (dd, J = 11.2, 4.5 Hz, 1H), 4.08 (dd, J = 11.3, 6.8 Hz, 1H), 4.05-3.93 (m, 1H), 3.91-3.74 (m, 3H), 3.39 (ddd, J = 13.9, 9.4, 3.7 Hz, 1H), 3.06 (dd, J = 17.7, 5.0 Hz, 1H), 2.92 (dd, J = 17.7, 5.8 Hz, 1H), 2.79 (s, 1H), 2.76- 2.6 (m, 1H), 2.61 (s, 3H), 2.37- | 684.2 |
| | 2.23 (m, 1H), 2.22-2.08 (m, 1H), | |
| | 1.98-1.80 (m, 2H), 1.84-1.76 (m, | |
| | 1H), 1.73 (s, 3H), 1.10 (d, J = 6.8 | |
| | Hz, 3H), 1.03 (d, J = 7.0 Hz, 3H). ¹³C | |
| | NMR (100 MHz, CDCl₃) δ 202.9, | |
| | 171.4, 166.4, 163.5, 160.2, 157.0, | |
| | 153.9, 147.3, 145.6, 144.2, 137.0, | |
| | 136.8, 134.0, 132.9, 125.1, 124.2, | |
| | 98.0, 78.01 68.3, 65.0, 59.8, 48.9, | |
| | 48.45 42.7, 41.1, 36.9, 34.0, 28.2, | |
| | 25.0, 19.0, 14.1, 12.7, 10.0. | |
| 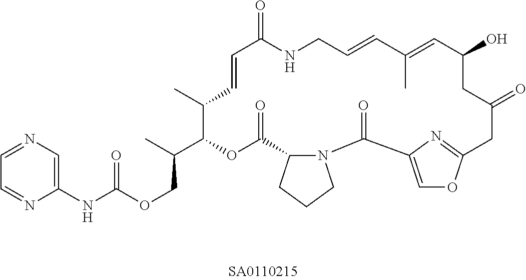 [SMILES: CC1=C\[C@@H](O)CC(=O)Cc2nc(co2)C(=O)N2CCC[C@@H]2C(=O)O[C@H]([C@H](C)COC(=O)Nc2cnccn2)[C@H](C)/C=C/C(=O)NC\C=C\1] | ¹H NMR (400 MHz, CDCl₃) δ 9.41 (br s, 1H), 9.28 (d, J = 1.5 Hz, 1H), 8.36 (s, 1H), 8.23 (d, J = 2.7 Hz, 1H), 8.19 (dd, J = 2.6, 1.5 Hz, 1H), 6.50 (dd, J = 16.3, 4.5 Hz, 1H), 6.14 (d, J = 15.6 Hz, 1H), 6.05 (dd, J = 9.0, 3.8 Hz, 1H), 5.80 (dd, J = 16.3, 2.0 Hz, 1H), 5.68 (ddd, J = 15.6, 9.2, 4.5 Hz, 1H), 5.53 (d, J = 8.6 Hz, 1H), 5.11 (dd, J = 9.9, 1.9 Hz, 1H), 4.93 (q, J = 6.2 Hz, 1H), 4.74 (d, J = 8.9, 3.0 Hz, 1H), 4.50- 4.35 (m, 2H), 4.09 (dd, J = 11.3, 6.9 Hz, 1H), 3.98 (dd, J = 11.5, 7.3 Hz, 1H), 3.90-3.75 (m, 3H), 3.42 (dd, J = 14.0, 9.3, 3.8 Hz, 1H), 3.07 (dd, J = 17.6, 5.0 Hz, 1H), 2.92 (dd, J = 17.7, 5.8 Hz, 1H), 2.86 (br | 665.2 |
| | s, 1H), 2.78-2.68 (m, 1H), 2.40- | |
| | 2.25 (m, 1H), 2.20-2.07 (m, 1H), | |
| | 1.97-1.85 (m, 2H), 1.84-1.74 (m, | |
| | 1H), 1.73 (s, 3H), 1.10 (d, J = 6.8 | |
| | Hz, 3H), 1.04 (d, J = 7.0 Hz, 3H). ¹³C | |
| | NMR (100 MHz, CDCl₃) δ 202.7, | |
| | 171.3, 166.2, 160.3, 157.2, 153.4, | |
| | 149.0, 145.1, 144.1, 141.9, 138.8, | |
| | 137.0, 136.7, 136.2, 134.0, 132.9, | |
| | 125.1, 124.2, 78.2, 68.6, 65.0, 59.8, | |
| | 49.0 48.6, 42.7, 41.1, 36.8, 33.9, | |
| | 28.3, 25.0, 14.0, 12.7, 10.0. | |
| 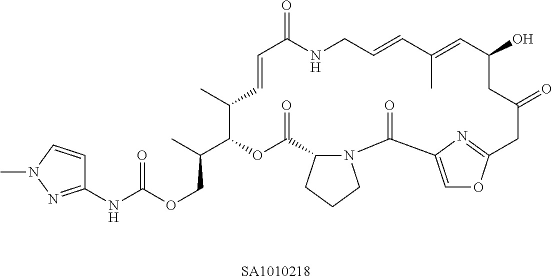 [SMILES: CC1=C\[C@@H](O)CC(=O)Cc2nc(co2)C(=O)N2CCC[C@@H]2C(=O)O[C@H]([C@H](C)COC(=O)Nc2ccn(C)n2)[C@H](C)/C=C/C(=O)NC\C=C\1] | ¹H NMR (400 MHz, CDCl₃) δ 9.01 (br s, 1H), 8.43 (s, 1H), 7.19 (d, J = 2.3 Hz, 1H), 6.50 (dd, J = 16.3, 4.4 Hz, 1H), 6.42 (s, 1H), 6.14 (d, J = 15.4 Hz, 2H), 5.79 (dd, J = 16.3, 1.9 Hz, 1H), 5.67 (ddd, J = 15.6, 9.3, 4.5 Hz, 1H), 5.51 (d, J = 8.6 Hz, 1H), 5.09 (dd, J = 9.7, 1.8 Hz, 1H), 4.93 (dd, J = 8.8, 5.2 Hz, 1H), 4.77 (dd, J = 8.9, 2.8 Hz, 1H), 4.46- 4.26 (m, 2H), 4.05-3.87 (m, 2H), 3.88-3.77 (m, 3H), 3.75 (s, 3H), 3.42 (ddd, J = 14.0, 9.2, 3.9 Hz, 1H), 3.07 (dd, J = 17.6, 5.1 Hz, 1H), 2.92 (br s, 1H), 2.91 (dd, J = 17.5, 5.9 Hz, 1H), 2.76-2.66 (m, 1H), 2.40-2.20 (m, 1H), 2.20-2.01 (m, 1H, 2.00-1.80 (m, 3H), 1.72 (s, | 667.2 |
| | 3H), 1.09 (d, J = 6.8 Hz, 3H), 0.99 | |
| | (d, J = 7.0 Hz, 3H). ¹³C NMR (100 | |
| | MHz, CDCl₃) δ 202.8, 171.3, 166.2, | |
| | 160.2, 157.0, 153.9, 147.6, 145.3 | |
| | 144.3, 137.0, 136.7, 134.0, 133.0, | |
| | 130.6, 125.1, 124.2, 96.1, 78.3, | |
| | 68.4, 65.0, 59.8, 49.0, 48.5, 42.8, | |
| | 41.0, 38.7, 36.9, 33.9, 28.3, 24.9, | |
| | 13.8, 12.7, 10.2. | |
| 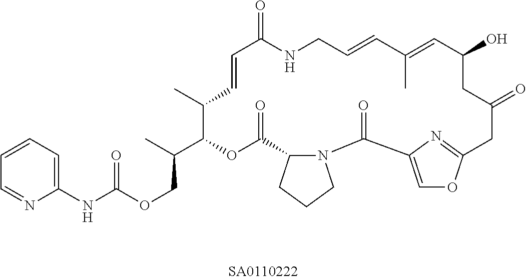 [SMILES: CC1=C\[C@@H](O)CC(=O)Cc2nc(co2)C(=O)N2CCC[C@@H]2C(=O)O[C@H]([C@H](C)COC(=O)Nc2ccccn2)[C@H](C)/C=C/C(=O)NC\C=C\1] | ¹H NMR (400 MHz, CDCl₃) δ 8.93 (br s, 1H), 8.34 (s, 1H), 8.23 (dd, J = 5.1, 1.8 Hz, 1H), 7.95 (d, J = 8.5 Hz, 1H), 7.64 (td, J = 8.4, 8.0, 1.9 Hz, 1H), 6.94 (dd, J = 7.3, 5.0 Hz, 1H), 6.49 (dd, J = 16.3, 4.6 Hz, 1H), 6.26-6.16 (m, 1H), 6.13 (d, J = 15.6 Hz, 1H), 5.79 (dd, J = 16.3, 1.9 Hz, 1H), 5.67 (ddd, J = 14.8, 9.3, 4.4 Hz, 1H), 5.49 (d, J = 8.7 Hz, 1H), 5.07 (dd, J = 10.0, 1.8 Hz, 1H), 4.91 (dt, J = 9.2, 5.5 Hz, 1H), 4.74 (dd, J = 8.8, 3.1 Hz, 1H), 4.42 (ddd, J = 13.9, 8.7, 4.5 Hz, 1H), 4.27 (dd, J = 11.2, 4.6 Hz, 1H), 4.15 (dd, J = 11.2, 5.7 Hz, 1H), 3.97 (dt, J = 11.4, 7.3 Hz, 1H), 3.88-3.71 (m, 3H), 3.40 (ddd, J = 13.8, 9.3, 3.7 Hz, | 664.4 |
| | 1H), 3.05 (dd, J = 17.6, 5.2 Hz, 1H), | |
| | 2.98-2.82 (m, 1H), 2.91 (dd, J = | |
| | 17.5, 5.7 Hz, 1H), 2.73 (ddd, J = | |
| | 9.1, 4.5, 2.1 Hz, 1H), 2.37-2.21 | |
| | (m, 1H), 2.23-2.12 (m, 1H), 1.98- | |
| | 1.84 (m, 3H), 1.72 (s, 3H), 1.08 (d, J = | |
| | 6.8 Hz, 3H), 1.04 (d, J = 7.0 Hz, | |
| | 3H). ¹³C NMR (100 MHz, CDCl₃) δ | |
| | 202.8, 171.5, 166.4, 160.2, 157.0, | |
| | 153.7, 152.1, 147.8, 145.1, 144.1, | |
| | 138.0, 137.0, 136.8, 134.0, 132.9, | |
| | 125.1, 124.2, 118.5, 112.6, 77.8, | |
| | 67.8, 65.0, 59.8, 48.9, 48.5, 42.8, | |
| | 41.0, 36.7, 34.1, 28.2, 25.0, 14.1, | |
| | 12.7, 10.0. | |
| 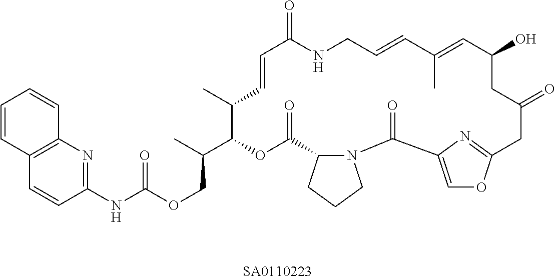 [SMILES: CC1=C\[C@@H](O)CC(=O)Cc2nc(co2)C(=O)N2CCC[C@@H]2C(=O)O[C@H]([C@H](C)COC(=O)Nc2ccc3ccccc3n2)[C@H](C)/C=C/C(=O)NC\C=C\1] | ¹H NMR (400 MHz, CDCl₃) δ 9.08 (br s, 1H), 8.20 (d, J = 9.0 Hz, 1H), 8.11 (d, J = 9.0 Hz, 1H), 7.77 (d, J = 8.5 Hz, 1H), 7.74 (dd, J = 8.1, 1.3 Hz, 1H), 7.63 (ddd, J = 8.5, 6.8, 1.5 Hz, 1H), 7.40 (ddd, J = 8.1, 6.9, 1.2 Hz, 1H), 6.48 (dd, J = 16.3, 4.6 Hz, 1H), 6.24-6.16 (m, 1H), 6.13 (d, J = 15.7 Hz, 1H), 5.79 (dd, J = 16.3, 1.9 Hz, 1H), 5.67 (ddd, J = 15.6, 9.3, 4.5 Hz, 1H), 5.50 (d, J = 8.7 Hz, 1H), 5.06 (dd, J = 10.1, 1.8 Hz, 1H), 4.91 (dt, J = 9.3, 5.4 Hz, 1H), 4.74 (dd, J = 8.7, 3.2 Hz, 1H), 4.45 (ddd, J = 13.9, 8.8, 4.5 Hz, 1H), 4.27 (d, J = 4.8 Hz, 2H), 4.01 (dt, J = 11.3, 7.3 Hz, 1H), 3.92- 3.73 (m, 3H), 3.38 (ddd, J = 14.6, | 714.5 |
| | 9.4, 3.6 Hz, 1H), 3.04 (dd, J = 17.6, | |
| | 5.1 Hz, 1H), 2.94 (dd, J = 17.6, 5.6 | |
| | Hz, 1H), 2.89 (s, 1H), 2.79-2.63 | |
| | (m, 1H), 2.38-2.23 (m, 1H), 2.20- | |
| | 2.10 (m, 1H), 2.01-1.82 (m, 3H), | |
| | 1.72 (d, J = 1.2 Hz, 3H), 1.13-1.07 | |
| | (m, 3H), 1.06 (s, 3H). ¹³C NMR (100 | |
| | MHz, CDCl₃) δ 202.8, 171.6, 166.6, | |
| | 160.2, 157.0, 154.0, 151.5, 146.8, | |
| | 145.5, 144.0, 138.2, 137.0, 136.9, | |
| | 133.9, 132.9, 129.7, 127.5, 127.14 | |
| | 125.7, 125.1, 124.6, 124.2, 113.3, | |
| | 77.6, 67.6, 65.1, 59.8, 48.9, 48.5, | |
| | 42.8, 41.1, 36.7, 34.1, 28.2, 25.2, | |
| | 14.4, 12.7, 9.9. | |
| 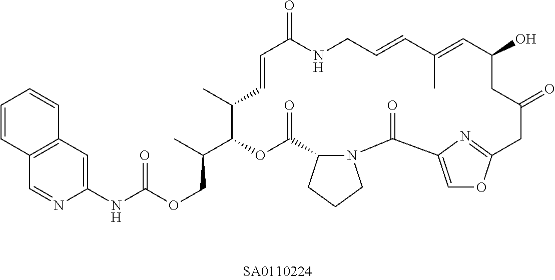 [SMILES: CC1=C\[C@@H](O)CC(=O)Cc2nc(co2)C(=O)N2CCC[C@@H]2C(=O)O[C@H]([C@H](C)COC(=O)Nc2cc3ccccc3cn2)[C@H](C)/C=C/C(=O)NC\C=C\1] | ¹H NMR (400 MHz, CDCl₃) δ 8.98 (s, 1H), 8.95 (s, 1H), 8.39 (s, 1H), 8.28 (s, 1H), 7.89-7.83 (m, 1H), 7.76 (d, J = 8.3 Hz, 1H), 7.60 (ddd, J = 8.2, 6.8, 1.2 Hz, 1H), 7.42 (ddd, J = 8.1, 6.8, 1.1 Hz, 1H), 6.50 (dd, J = 16.3, 4.6 Hz, 1H), 6.15 (broad t, J = 4.7 Hz, 1H), 6.12 (d, J = 8.2 Hz, 1H), 5.80 (dd, J = 16.3, 1.9 Hz, 1H), 5.68 (ddd, J = 15.5, 9.3, 4.5 Hz, 1H), 5.50 (d, J = 8.7 Hz, 1H), 5.10 (dd, J = 10.0, 1.8 Hz, 1H), 4.92 (dt, J = 8.6, 5.4 Hz, 1H), 4.78 (dd, J = 8.8, 3.1 Hz, 1H), 4.44 (ddd, J = 14.0, 8.9, 4.5 Hz, 1H), 4.32 (dd, J = 11.2, 4.7 Hz, 1H), 4.22 (dd, J = 11.2, 5.6 Hz, 1H), 3.99 (dt, J = 11.4, | 714.5 |
| | 7.4 Hz, 1H), 3.89-3.73 (m, 3H), | |
| | 3.39 (ddd, J = 14.1, 9.3, 3.6 Hz, | |
| | 1H), 3.06 (dd, J = 17.6, 5.1 Hz, 1H), | |
| | 2.92 (dd, J = 17.6, 5.7 Hz, 1H), 2.74 | |
| | (ddt, J = 6.9, 4.5, 2.1 Hz, 1H), 2.41- | |
| | 2.26 (m, 1H), 2.23-2.10 (m, 1H), | |
| | 2.01-1.75 (m, 4H), 1.72 (d, J = 1.2 | |
| | Hz, 3H), 1.10 (d, J = 6.8 Hz, 3H), | |
| | 1.07 (d, J = 7.0 Hz, 3H). ¹³C NMR | |
| | (100 MHz, CDCl₃) δ 202.8, 171.6, | |
| | 166.4, 160.2, 157.0, 153.8, 151.0, | |
| | 147.2, 145.19, 144.1, 138.0, 137.1, | |
| | 136.8, 134.0, 132.9, 130.5, 127.4, | |
| | 126.5, 125.90, 125.2, 125.1, 124.2, | |
| | 106.2, 77.9, 67.8, 65.1, 59.8, 48.9, | |
| | 48.5, 42.8, 41.1, 36.7, 34.2, 28.3, | |
| | 25.1, 14.2, 12.7, 10.03. | |
| 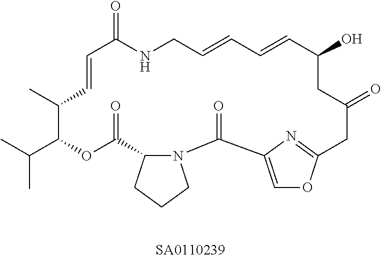 [SMILES: CC(C)[C@H]1OC(=O)[C@H]2CCCN2C(=O)c2coc(n2)CC(=O)C[C@H](O)/C=C/C=C/CNC(=O)/C=C/[C@H]1C] | ¹H NMR (400 MHz, CDCl₃) δ 8.14 (s, 1H), 6.48 (dd, J = 16.4, 4.8 Hz, 1H), 6.27-6.15 (m, 1H), 6.15- 6.04 (m, 2H), 5.78 (dd, J = 16.4, 2.1 Hz, 1H), 5.73-5.58 (m, 2H), 4.71 (ddd, J = 12.7, 9.3, 2.8 Hz, 2H), 4.60 (q, J = 6.3 Hz, 1H), 4.33 (ddd, J = 14.4, 8.1, 5.0 Hz, 1H), 4.10- 3.96 (m, 1H), 3.95-3.85 (m, 1H), 3.84 (s, 2H), 3.48 (ddd, J = 15.0, 7.9, 3.5 Hz, 1H), 3.01 (dd, J = 16.8, 5.4 Hz, 1H), 2.91 (dd, J = 16.9, 5.7 Hz, 1H), 2.80-2.71 (m, 1H), 2.68 (br s, 1H) 2.23-2.10 (m, 1H), 2.01- 1.79 (m, 4H), 1.05 (d, J = 6.7 Hz, 3H), 0.99 (d, J = 6.3 Hz, 3H), 0.95 (d, J = 6.6 Hz, 3H). ¹³C NMR (100 | 514.3 |
| | MHz, CDCl₃) δ 202.4, 171.7, 166.7, | |
| | 160.1, 157.0, 144.6, 144.1, 137.2, | |
| | 133.9, 131.6, 130.4, 130.1, 124.1, | |
| | 81.6, 69.0, 59.6, 48.9, 48.6, 43.1, | |
| | 40.9, 36.7, 29.4, 28.4, 25.1, 19.7, | |
| | 18.7, 10.2. | |
| 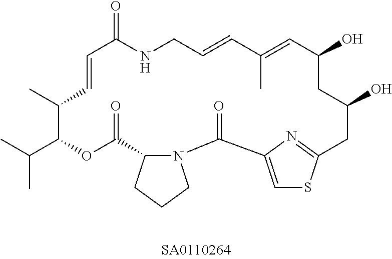 [SMILES: CC1=C\[C@@H](O)C[C@@H](O)Cc2nc(cs2)C(=O)N2CCC[C@@H]2C(=O)O[C@H](C(C)C)[C@H](C)/C=C/C(=O)NC\C=C\1] | ¹H NMR (400 MHz, CDCl₃) δ 8.06 (s, 1H), 6.52 (dd, J = 16.3, 4.3 Hz, 1H), 6.19 (d, J = 15.5 Hz, 1H), 5.93 (dd, J = 8.7, 3.9 Hz, 1H), 5.80 (dd, J = 16.3, 2.0 Hz, 1H), 5.65 (ddd, J = 15.5, 9.4, 4.1 Hz, 1H), 5.38 (d, J = 9.2 Hz, 1H), 4.95-4.71 (m, 3H), 4.46 (ddd, J = 13.5, 8.6, 4.1 Hz, 1H), 4.33 (td, J = 8.1, 4.0 Hz, 1H), 4.07 (ddd, J = 12.5, 8.0, 4.5 Hz, 1H), 3.86 (dt, J = 11.1, 7.2 Hz, 1H), 3.44 (ddd, J = 14.2, 9.4, 3.8 Hz, 1H), 3.17 (dd, J = 16.3, 7.5 Hz, 1H), 3.04 (dd, J = 16.3, 3.7 Hz, 1H), 2.97 (br s, 1H), 2.83-2.68 (m, 1H), 2.20-2.05 (m, 2H), 2.05- 1.83 (m, 3H), 1.78 (s, 3H), 1.76- | 546.3 |
| | 1.60 (m, 2H), 1.10 (d, J = 6.9 Hz, | |
| | 3H), 1.01 (d, J = 6.5 Hz, 3H), 0.95 | |
| | (d, J = 6.7 Hz, 3H). ¹³C NMR (100 | |
| | MHz, CDCl₃) δ 171.8, 166.4, 166.3, | |
| | 161.0, 149.7, 145.0, 136.6, 134.8, | |
| | 134.0, 126.6, 125.2, 123.7, 81.0, | |
| | 68.4, 66.9, 59.7, 49.3, 42.6, 41.4, | |
| | 40.8, 36.6, 29.4, 28.4, 25.3, 19.9, | |
| | 18.6, 13.0, 10.0. | |
| 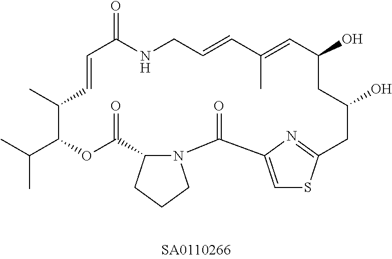 [SMILES: CC1=C\[C@@H](O)C[C@H](O)Cc2nc(cs2)C(=O)N2CCC[C@@H]2C(=O)O[C@H](C(C)C)[C@H](C)/C=C/C(=O)NC\C=C\1] | ¹H NMR (400 MHz, CDCl₃) δ 7.96 (s, 1H), 6.51 (dd, J = 16.2, 4.5 Hz, 1H), 6.20 (dd, J = 15.6, 1.1 Hz, 1H), 6.14 (dd, J = 8.6, 4.0 Hz, 1H), 5.76 (dd, J = 16.2, 2.0 Hz, 1H), 5.72- 5.66 (m, 1H), 5.66-5.59 (m, 1H), 4.86 (dt, J = 9.6, 4.9 Hz, 1H), 4.77- 4.66 (m, 2H), 4.42-4.28 (m, 2H), 4.04 (br s, 1H), 3.92-3.72 (m, 2H), 3.44 (ddd, J = 14.0, 9.4, 4.1 Hz, 1H), 3.23-3.02 (m, 3H), 2.74 (dqd, J = 7.4, 4.8, 2.6 Hz, 1H), 2.12 (dq, J = 13.2, 8.1, 7.3 Hz, 1H), 2.05-1.86 (m, 4H), 1.85-1.78 (m, 2H), 1.77 (d, J = 1.2 Hz, 3H), 1.07 (d, J = 6.8 Hz, 3H), 1.01 (d, J = 6.5 Hz, 3H), | 546.2 |
| | 0.94 (d, J = 6.8 Hz, 3H). ¹³C NMR | |
| | (100 MHz, CDCl₃) δ 172.0, 166.5, | |
| | 166.1, 161.3, 150.3, 145.1, 137.3, | |
| | 133.9, 133.4, 125.8, 125.0, 123.7, | |
| | 81.3, 68.6, 66.7, 59.6, 49.2, 41.9, | |
| | 41.1, 40.47, 36.7, 29.4, 28.5, 25.5, | |
| | 19.8, 18.6, 12.8, 9.7. | |
| 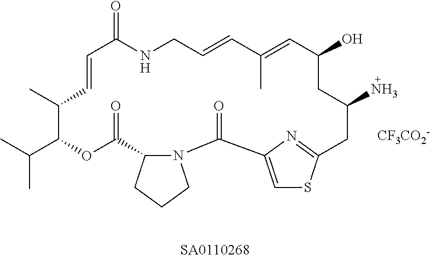 | ¹H NMR (400 MHz, MeOD) δ 8.12 (s, 1H), 6.83 (dd, J = 15.7, 4.4 Hz, 1H), 6.31 (dd, J = 15.5, 1.0 Hz, 1H), 5.88 (dd, J = 15.7, 2.0 Hz, 1H), 5.77-5.66 (m, 1H), 5.50 (d, J = 8.6 Hz, 1H), 4.90-4.80 (m, 1H), 4.82- 4.71 (m, 2H), 4.11 (dd, J = 14.3, 9.1 Hz, 1H), 3.92-3.82 (m, 2H), 3.77 (dt, J = 11.2, 7.6 Hz, 1H), 3.61- 3.48 (m, 2H), 3.37 (dd, J = 15.6, 6.9 Hz, 1H), 2.89-2.77 (m, 1H), 2.45 (ddd, J = 14.7, 5.1, 3.1 Hz, 1H), 2.22-2.07 (m, 1H), 2.03-1.90 (m, 3H), 1.83 (d, J = 1.2 Hz, 3H), 1.81- 1.72 (m, 2H), 1.63-1.50 (m, 1H), 1.14 (d, J = 6.8 Hz, 3H), 1.00 (d, J = 6.9 Hz, 3H), 0.97 (d, J = 6.5 Hz, | 545.5 |
| | 3H). ¹³C NMR (100 MHz, MeOD) δ | |
| | 172.1, 167.5, 164.0, 163.0, 151.9, | |
| | 148.34 138.7, 136.3, 134.9, 128.0, | |
| | 126.3, 124.2, 82.4, 67.0, 61.1, | |
| | 50.98, 50.95, 41.3, 39.8, 38.0, 35.5, | |
| | 30.7, 29.8, 26.4, 20.2, 18.8, 13.2, | |
| | 9.6. | |
| 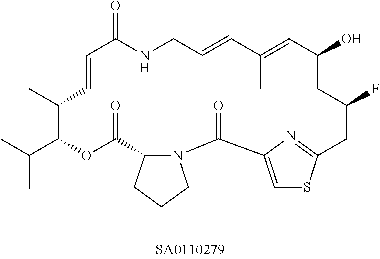 [SMILES: CC1=C\[C@@H](O)C[C@@H](F)Cc2nc(cs2)C(=O)N2CCC[C@@H]2C(=O)O[C@H](C(C)C)[C@H](C)/C=C/C(=O)NC\C=C\1] | ¹H NMR (400 MHz, CDCl₃) δ 7.95 (s, 1H), 6.51 (dd, J = 16.4, 4.2 Hz, 1H), 6.23 (d, J = 15.7 Hz, 1H), 5.96 (dd, J = 8.9, 3.5 Hz, 1H), 5.81 (dd, J = 16.3, 2.0 Hz, 1H), 5.68 (ddd, J = 15.9, 9.3, 4.1 Hz, 1H), 5.39 (d, J = 9.2 Hz, 1H), 5.34-5.10 (m, J_{HF} = 47.9, 1H), 4.88-4.71 (m, 3H), 4.55 (ddd, J = 14.2, 9.0, 4.0 Hz, 1H), 4.06 (dt, J = 11.6, 6.4 Hz, 1H), 3.78 (dt, J = 11.6, 6.3 Hz, 1H), 3.52-3.31 (m, 2H), 3.10 (ddd, J = 27.6, 16.4, 4.1 Hz, 1H), 2.82-2.68 (m, 1H), 2.29-2.09 (m, 2H), 2.00- 1.80 (m, 5H), 1.78 (s, 3H), 1.10 (d, J = 6.7 Hz, 3H), 1.02 (d, J = 6.3 Hz, 3H), 0.95 (d, J = 6.5 Hz, 3H). ¹³C | 548.3 |
| | NMR (100 MHz, CDCl₃) δ 172.2, | |
| | 166.5, 163.9 (d, J = 4.3 Hz), 161.5, | |
| | 150.1, 144.8, 136.2, 135.8, 133.4, | |
| | 125.48, 125.2, 123.8, 90.0 (d, J = | |
| | 169.9 Hz), 80.9, 65.6, 59.2, 49.3, | |
| | 42.1 (d, J = 20.6 Hz), 41.3, 38.5 (d, | |
| | J = 22.8 Hz), 36.6, 29.4, 28.5, 25.4, | |
| | 19.9, 18.5, 12.8, 9.8. | |
| 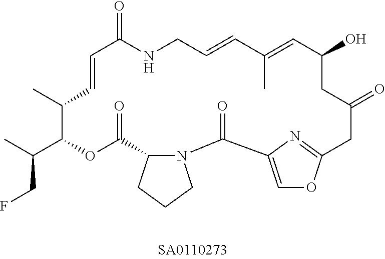 [SMILES: CC1=C\[C@@H](O)CC(=O)Cc2nc(co2)C(=O)N2CCC[C@@H]2C(=O)O[C@H]([C@H](C)CF)[C@H](C)/C=C/C(=O)NC\C=C\1] | ¹H NMR (400 MHz, CDCl₃) δ 8.11 (s, 1H), 6.49 (dd, J = 16.4, 5.0 Hz, 1H), 6.48-6.40 (m, 1H), 6.13 (d, J = 15.7 Hz, 1H), 5.83 (d, J = 16.4 Hz, 1H), 5.72 (ddd, J = 14.8, 9.1, 4.6 Hz, 1H), 5.42 (d, J = 8.8 Hz, 1H), 5.03 (d, J = 10.4 Hz, 1H), 4.93 (dt, J = 9.6, 5.6 Hz, 1H), 4.69 (dd, J = 8.9, 3.2 Hz, 1H), 4.55-4.30 (m, 3H), 4.01 (dt, J = 11.5, 7.2 Hz, 1H), 3.84 (s, 2H), 3.81-3.71 (m, 1H), 3.41 (ddd, J = 13.8, 9.2, 3.7 Hz, 1H), 3.07 (dd, J = 17.0, 5.9 Hz, 1H), 3.03-2.95 (m, 1H), 2.90 (dd, J = 17.0, 5.1 Hz, 1H), 2.75 (br t, J = 6.6 Hz, 1H), 2.25-2.11 (m, 2H), 2.00- | 546.2 |
| | 1.84 (m, 3H), 1.74 (s, 3H), 1.09 (d, J = | |
| | 6.9 Hz, 6H). ¹³C NMR (100 MHz, | |
| | CDCl₃) δ 202.3, 171.6, 166.5, | |
| | 160.2, 156.8, 144.0, 143.6, 137.0, | |
| | 134.4, 132.6, 125.3, 125.3, 124.6, | |
| | 85.3 (d, J_{CF} = 170.4 Hz), 76.1 (d, | |
| | J_{CF} = 4.8 Hz), 65.2, 59.6, 52.1, 48.8, | |
| | 48.5, 43.3, 40.9, 36.4, 35.8 (d, J_{CF} = | |
| | 19.1 Hz), 29.7, 28.3, 25.13, 25.11, | |
| | 20.2, 13.5, 13.0 (d, J_{CF} = 5.2 Hz), | |
| | 12.7, 10.1. | |
| 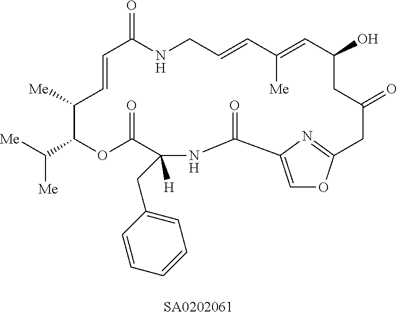 [SMILES: CC1=C\[C@@H](O)CC(=O)Cc2nc(co2)C(=O)N[C@H](Cc2ccccc2)C(=O)O[C@H](C(C)C)[C@H](C)/C=C/C(=O)NC\C=C\1] | ¹H NMR (300 MHz, Chloroform-d) δ 8.04 (s, 1H), 7.27- 7.19 (m, 3H), 7.16 (dd, J = 7.8, 1.8 Hz, 2H), 6.49 (dd, J = 16.2, 5.6 Hz, 1H), 6.12 (d, J = 15.7 Hz, 1H), 5.97 (d, J = 6.6 Hz, 1H), 5.84 (dd, J = 16.2, 1.7 Hz, 1H), 5.71-5.54 (m, 1H), 5.43 (d, J = 8.9 Hz, 1H), 4.94 (tdd, J = 8.9, 5.9, 3.6 Hz, 2H), 4.78 (dd, J = 10.2, 1.9 Hz, 1H), 4.41- 4.28 (m, 1H), 3.87 (d, J = 16.8 Hz, 1H), 3.77 (d, J = 16.8 Hz, 1H), 3.69 (ddd, J = 15.8, 7.1, 3.9 Hz, 1H), 3.32 (dd, J = 14.3, 5.2 Hz, 1H), 3.04- 2.84 (m, 3H), 2.84-2.70 (m, 1H), 2.43 (s, 1H), 2.08-1.92 (m, 1H), 1.73 (d, J = 1.2 Hz, 3H), 1.09 (d, J = 6.9 Hz, 3H), 1.00-0.91 (m, 6H). | |
| 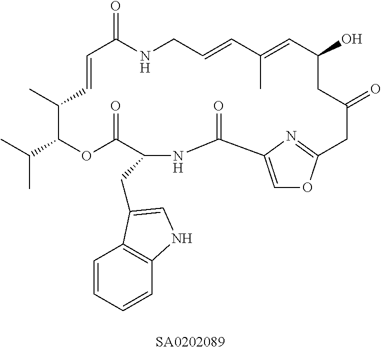 [SMILES: CC1=C\[C@@H](O)CC(=O)Cc2nc(co2)C(=O)N[C@H](Cc2c[nH]c3ccccc23)C(=O)O[C@H](C(C)C)[C@H](C)/C=C/C(=O)NC\C=C\1] | ¹H NMR (300 MHz, Chloroform-d) δ 8.20 (s, 1H), 8.08 (s, 1H), 7.49 (d, J = 8.0 Hz, 1H), 7.41 (d, J = 8.5 Hz, 1H), 7.20 (dt, J = 8.1, 1.0 Hz, 1H), 7.10 (dd, J = 7.0, 1.2 Hz, 1H), 7.05 (dd, J = 3.2, 1.9 Hz, 1H), 6.97 (ddd, J = 8.0, 7.0, 1.1 Hz, 1H), 6.58 (dd, J = 16.0, 5.6 Hz, 1H), 6.02-5.79 (m, 3H), 5.69 (d, J = 15.6 Hz, 1H), 5.09 (td, J = 8.4, 4.3 Hz, 2H), 4.85 (dd, J = 10.2, 1.8 Hz, 1H), 4.75 (q, J = 7.0 Hz, 1H), 4.44 (d, J = 12.7 Hz, 1H), 3.90-3.60 (m, 3H), 3.56-3.43 (m, 1H), 3.27 (dd, J = 15.2, 5.4 Hz, 1H), 2.86-2.65 (m, 2H), 2.54 (dd, J = 16.4, 5.5 Hz, 1H), 2.44-2.26 (m, 1H), 2.11-1.89 (m, 1H), 1.71 (d, J = 1.2 Hz, 3H), 1.11 (d, J = 6.9 Hz, 3H), 0.97 (d, J = 6.8 Hz, 3H), 0.93 (d, J = 6.8 Hz, 3H). | |
| 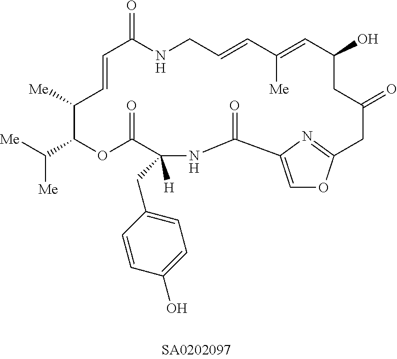 [SMILES: CC1=C\[C@@H](O)CC(=O)Cc2nc(co2)C(=O)N[C@H](Cc2ccc(O)cc2)C(=O)O[C@H](C(C)C)[C@H](C)/C=C/C(=O)NC\C=C\1] | ¹H NMR (300 MHz, Chloroform-d) δ 8.10 (s, 1H), 7.21 (d, J = 8.3 Hz, 1H), 7.01-6.87 (m, 2H), 6.76-6.63 (m, 2H), 6.49 (dd, J = 16.2, 4.9 Hz, 1H), 6.08 (d, J = 15.7 Hz, 1H), 5.96-5.78 (m, 2H), 5.53 (ddd, J = 15.7, 8.1, 3.7 Hz, 1H), 5.42 (d, J = 8.9 Hz, 1H), 5.01- 4.85 (m, 2H), 4.74 (dd, J = 10.2, 1.7 Hz, 1H), 4.50-4.31 (m, 1H), 3.88 (d, J = 17.2 Hz, 1H), 3.84-3.74 (m, 1H), 3.63-3.50 (m, 1H), 3.22 (dd, J = 14.3, 5.2 Hz, 1H), 2.95 (dd, J = 12.9, 6.6 Hz, 3H), 2.79 (d, J = 8.7 Hz, 2H), 2.02 (dt, J = 10.0, 6.6 Hz, 1H), 1.69 (d, J = 1.2 Hz, 3H), 1.13 (d, J = 6.8 Hz, 3H), 0.96 (td, J = 7.0, 3.7 Hz, 6H). | |

### Example 3: Activity Data

TABLE 2
Minimal Inhibitory Concentration (MIC) Values (μg/mL) for Gram-Positive bacteria:

| | Gram-Positive Organisms | | | | | | | | | |
|---|---|---|---|---|---|---|---|---|---|---|
| | | | | | S. aureus | | | | S. aureus | |
| | | E. faecium | E. faecium | | Vat A | | | | Linezolid- | |
| | | ATCC | VRE; | | strepto- | | | | resistant; | |
| Compound | E. faecalis | 35667 | vanA | S. aureus | gramin | S. aureus | S. aureus | S. aureus | cfr | S. pneumoniae |
| virginiamycin M1 | 32 | 2 | 4 | 4 | 32 | 2 | 8 | 2 | >64 | 4 |
| virginiamycin M2 | >64 | 4 | 8 | 16 | >64 | 16 | 32 | 8 | >64 | 8 |
| madumycin I | 32 | 2 | 4 | 8 | 64 | 8 | 8 | 4 | 64 | 4 |
| maduycin II | >64 | 0.5 | 8 | 8 | >64 | 8 | 16 | 8 | >64 | 4 |
| flopristin | >64 | 0.5 | 2 | 0.5 | 8 | 0.5 | 1 | 0.5 | >64 | 2 |
| SA0110016 | 32 | 32 | 64 | 64 | 64 | 64 | 64 | 64 | 64 | 16 |
| SA0110026 | >64 | 64 | >64 | >64 | >64 | >64 | >64 | >64 | >64 | 64 |
| SA0110036 | >64 | 8 | 32 | 32 | >64 | 32 | >64 | 16 | >64 | 16 |
| SA0110037 | >64 | 16 | 64 | 32 | >64 | 32 | 64 | 16 | >64 | 16 |
| SA0110040 | >64 | 16 | 64 | 32 | >64 | 32 | 64 | 16 | >64 | 16 |
| SA0110041 | >64 | 8 | >64 | >64 | >64 | >64 | >64 | 32 | >64 | >64 |
| SA0110043 | >64 | 16 | 64 | 32 | >64 | 32 | 64 | 32 | >64 | 16 |
| SA0110044 | >64 | 16 | >64 | >64 | >64 | >64 | >64 | 32 | >64 | >64 |
| SA0110067 | >64 | 32 | >64 | >64 | >64 | >64 | >64 | 64 | >64 | 32 |
| SA0110081 | >64 | 32 | >64 | >64 | >64 | >64 | >64 | 32 | >64 | 32 |
| SA0110082 | >64 | 32 | >64 | >64 | >64 | >64 | >64 | 32 | >64 | 8 |
| SA0110095 | >64 | 32 | >64 | >64 | >64 | >64 | >64 | 64 | >64 | 16 |
| SA0110098 | >64 | 16 | 64 | 64 | >64 | 32 | 64 | 32 | >64 | 16 |
| SA0110101 | >64 | 64 | >64 | >64 | >64 | >64 | >64 | >64 | >64 | 32 |
| SA0110113 | >64 | 64 | >64 | >64 | >64 | >64 | >64 | >64 | >64 | 8 |
| SA0110115 | >64 | 32 | >64 | >64 | >64 | >64 | >64 | 64 | >64 | 16 |
| SA0110117 | >64 | 16 | 32 | 16 | >64 | 16 | 32 | 16 | >64 | 4 |
| SA0110118 | >64 | 4 | 16 | 8 | >64 | 8 | 16 | 8 | >64 | 2 |
| SA0110120 | >64 | 32 | 64 | 64 | >64 | 64 | >64 | 32 | >64 | 16 |
| SA0110141 | >64 | 4 | 8 | 16 | 64 | 16 | 16 | 8 | >64 | 8 |
| SA0202089 | >64 | 32 | 32 | 32 | 64 | 32 | 32 | 32 | >64 | 32 |
| SA0202061 | 32 | 32 | 64 | 64 | 64 | 64 | 64 | 64 | >64 | 16 |
| SA0306004 | >64 | 32 | >64 | 32 | >64 | 64 | 64 | 32 | >64 | 64 |
| SA0110161 | >64 | >64 | >64 | >64 | >64 | >64 | >64 | >64 | >64 | >64 |
| SA0110170 | >64 | >64 | >64 | >64 | >64 | >64 | >64 | >64 | >64 | 64 |
| SA0110180 | >64 | 16 | 16 | 32 | >64 | 16 | 32 | 16 | >64 | 32 |
| SA0110184 | >64 | 16 | 16 | 16 | >64 | 16 | 32 | 16 | >64 | 32 |
| SA0110185 | >64 | 16 | 32 | 32 | >64 | 16 | 32 | 16 | >64 | 32 |
| SA0110193 | >64 | 8 | >64 | 8 | 16 | 8 | 16 | 8 | >64 | >64 |
| SA0110195 | >64 | 8 | >64 | 16 | >64 | 16 | 16 | 8 | >64 | >64 |
| SA0110196 | >64 | 16 | 64 | 32 | >64 | 32 | 64 | 16 | >64 | 32 |
| SA0110205 | >64 | 32 | 64 | >64 | >64 | >64 | >64 | >64 | >64 | 32 |
| SA0110206 | >64 | 8 | 16 | 16 | >64 | 16 | 32 | 16 | >64 | 8 |
| SA0110209 | >64 | 16 | >64 | 16 | >64 | 16 | >64 | 16 | >64 | 16 |
| SA0110210 | >64 | 64 | 64 | >64 | >64 | >64 | >64 | >64 | >64 | 64 |
| SA0110214 | >64 | 32 | 64 | 64 | >64 | 64 | >64 | 64 | >64 | 32 |
| SA0110215 | >64 | 64 | >64 | >64 | >64 | >64 | >64 | >64 | >64 | 64 |
| SA0110218 | >64 | >64 | >64 | >64 | >64 | >64 | >64 | >64 | >64 | >64 |
| SA0110225 | >64 | 32 | 64 | >64 | >64 | 64 | >64 | 64 | >64 | 32 |
| SA0110222 | >64 | 32 | 64 | >64 | >64 | >64 | >64 | 64 | >64 | 32 |
| SA0110223 | >64 | 4 | 16 | 8 | 32 | 8 | 16 | 4 | >64 | 32 |
| SA0110224 | >64 | 4 | 8 | 4 | 16 | 4 | 8 | 4 | >64 | 16 |
| SA0110239 | >64 | >64 | >64 | >64 | >64 | >64 | >64 | >64 | >64 | 64 |
| SA0110241 | >64 | >64 | >64 | >64 | >64 | >64 | >64 | >64 | >64 | >64 |
| SA0110252 | >64 | >64 | >64 | >64 | >64 | >64 | >64 | >64 | >64 | >64 |
| SA0110261 | >64 | 64 | >64 | >64 | >64 | 64 | >64 | 64 | >64 | 64 |
| SA0110264 | >64 | 1 | 4 | 8 | >64 | 16 | 16 | 8 | >64 | 2 |
| SA0110266 | >64 | >64 | >64 | >64 | >64 | >64 | >64 | >64 | >64 | >64 |
| SA0110273 | >64 | 16 | 64 | 64 | >64 | 64 | 64 | 32 | >64 | 16 |
| SA0110268 | >64 | 64 | >64 | >64 | >64 | >64 | >64 | >64 | >64 | 32 |
| SA0110279 | >64 | 0.5 | 1 | 0.5 | 8 | 0.5 | 1 | 0.5 | >64 | 1 |
| SA0111044 | >64 | >64 | >64 | >64 | >64 | >64 | >64 | >64 | >64 | >64 |
| SA0202097 | >64 | >64 | >64 | >64 | >64 | >64 | >64 | >64 | >64 | 64 |
| SA0202094 | >64 | >64 | >64 | >64 | >64 | >64 | >64 | >64 | >64 | >64 |
| levofloxacin | 1 | 4 | >8 | 0.25 | 1 | 0.12 | 0.25 | 2 | 8 | 0.5 |
| (positive | | | | | | | | | | |
| control) | | | | | | | | | | |

TABLE 2
Minimal Inhibitory Concentration (MIC) Values (μg/mL) for Gram-Negative bacteria:

| | Gram-Negative Organisms | | | | | | |
|---|---|---|---|---|---|---|---|
| | | | | E. coli | E. coli | E. coli | |
| | A. baumannii | E. coli | E. coli | permeability | BAS2006, | ΔompC768; | |
| | ATCC | ATCC | olC efflux | mutant BAS1314; | OMP C, | KanR | K. pneumoniae |
| Compound | 19606 | 25922 | defective | IMP4213ZAB::TN10 | OMP F | insert | ATCC 43816 |
| virginiamycin M1 | >64 | >64 | 2 | 0.25 | >64 | nt | >64 |
| virginiamycin M2 | >64 | >64 | 2 | 2 | 2 | nt | >64 |
| madumycin I | >64 | >64 | 2 | 0.25 | 0.5 | nt | >64 |
| madumycin II | >64 | >64 | 2 | 0.5 | 1 | nt | >64 |
| flopristin | >64 | 32 | 1 | <0.06 | 0.25 | 64 | >64 |
| SA0110016 | >64 | >64 | 8 | 16 | >64 | nt | >64 |
| SA0110026 | >64 | >64 | 32 | 64 | 64 | nt | >64 |
| SA0110036 | >64 | >64 | 2 | 8 | 8 | nt | >64 |
| SA0110037 | >64 | >64 | 4 | 8 | 16 | nt | >64 |
| SA0110040 | >64 | >64 | 2 | 8 | 16 | nt | >64 |
| SA0110041 | >64 | >64 | 8 | 8 | >64 | nt | >64 |
| SA0110043 | >64 | >64 | 2 | 8 | 16 | nt | >64 |
| SA0110044 | >64 | >64 | 16 | 16 | >64 | nt | >64 |
| SA0110067 | >64 | >64 | 8 | 32 | 64 | nt | >64 |
| SA0110081 | >64 | >64 | 4 | 32 | 32 | nt | >64 |
| SA0110082 | >64 | >64 | 4 | 16 | 32 | nt | >64 |
| SA0110095 | >64 | >64 | 8 | 16 | 32 | nt | >64 |
| SA0110098 | >64 | >64 | 2 | 8 | 16 | nt | >64 |
| SA0110101 | >64 | >64 | 8 | 16 | 64 | nt | >64 |
| SA0110113 | >64 | >64 | 8 | 16 | 64 | nt | >64 |
| SA0110115 | >64 | >64 | 8 | 16 | 64 | nt | >64 |
| SA0110117 | >64 | >64 | 2 | 4 | 8 | nt | >64 |
| SA0110118 | >64 | >64 | 2 | 2 | 8 | nt | >64 |
| SA0110120 | >64 | >64 | 4 | 8 | 32 | nt | >64 |
| SA0110141 | >64 | >64 | 2 | 1 | 1 | nt | >64 |
| SA0202089 | >64 | >64 | 16 | 8 | 32 | nt | >64 |
| SA0202061 | >64 | >64 | 4 | 4 | 16 | nt | >64 |
| SA0306004 | >64 | >64 | 4 | 4 | 4 | nt | >64 |
| SA0110161 | >64 | >64 | >64 | >64 | >64 | >64 | >64 |
| SA0110170 | >64 | >64 | 32 | 32 | >64 | >64 | >64 |
| SA0110180 | >64 | >64 | 4 | 4 | >64 | >64 | >64 |
| SA0110184 | >64 | >64 | 4 | 2 | >64 | >64 | >64 |
| SA0110185 | >64 | >64 | 2 | 4 | 64 | >64 | >64 |
| SA0110193 | >64 | >64 | 8 | 4 | >64 | >64 | >64 |
| SA0110195 | >64 | >64 | 8 | 8 | >64 | >64 | >64 |
| SA0110196 | >64 | >64 | 2 | 8 | 32 | >64 | >64 |
| SA0110205 | >64 | >64 | 8 | 16 | >64 | >64 | >64 |
| SA0110206 | >64 | >64 | 2 | 2 | 16 | >64 | >64 |
| SA0110209 | >64 | >64 | 2 | 2 | >64 | >64 | >64 |
| SA0110210 | >64 | >64 | 8 | 8 | >64 | >64 | >64 |
| SA0110214 | >64 | >64 | 8 | 8 | 64 | >64 | >64 |
| SA0110215 | >64 | >64 | 8 | 16 | >64 | >64 | >64 |
| SA0110218 | >64 | >64 | 16 | 32 | >64 | >64 | >64 |
| SA0110225 | >64 | >64 | 4 | 8 | >64 | >64 | >64 |
| SA0110222 | >64 | >64 | 4 | 16 | 64 | >64 | >64 |
| SA0110223 | >64 | >64 | 2 | 2 | 16 | >64 | >64 |
| SA0110224 | >64 | >64 | 1 | 1 | 8 | >64 | >64 |
| SA0110239 | >64 | >64 | 8 | 8 | 64 | >64 | >64 |
| SA0110241 | >64 | >64 | >64 | >64 | >64 | >64 | >64 |
| SA0110252 | >64 | >64 | >64 | >64 | >64 | >64 | >64 |
| SA0110261 | >64 | >64 | 64 | 16 | >64 | >64 | >64 |
| SA0110264 | >64 | >64 | 2 | 1 | 4 | >64 | >64 |
| SA0110266 | >64 | >64 | 64 | 16 | >64 | >64 | >64 |
| SA0110273 | >64 | >64 | 4 | 8 | 16 | >64 | >64 |
| SA0110268 | >64 | >64 | 2 | 2 | 16 | >64 | >64 |
| SA0110279 | >64 | 32 | 1 | <0.06 | 0.25 | 32 | >64 |
| SA0111044 | >64 | >64 | >64 | >64 | >64 | >64 | >64 |
| SA0202097 | >64 | >64 | 32 | 32 | >64 | >64 | >64 |
| SA0202094 | >64 | >64 | >64 | 32 | >64 | >64 | >64 |
| levofloxacin | 0.25 | 0.015 | <0.003 | <0.008 | <0.008 | 0.015 | 0.03 |
| (positive | | | | | | | |
| control) | | | | | | | |

TABLE 3
Minimal Inhibitory Concentration (MIC) Values
(μg/mL) for Gram-Negative bacteria:

| | Gram-Negative Organisms | | | |
|---|---|---|---|---|
| | K. pneumoniae | | P. aeruginosa | |
| | multidrug- | P. aeruginosa | MDR clinical | H. influenzae |
| Compound | resistant | ATCC 27853 | isolate | ATCC 49247 |
| virginiamycin M1 | >64 | >64 | >64 | 1 |
| virginiamycin M2 | >64 | >64 | >64 | 16 |
| madumycin I | >64 | >64 | >64 | 8 |
| madumycin II | >64 | >64 | >64 | 1 |
| flopristin | >64 | >64 | >64 | 0.25 |
| SA0110016 | >64 | >64 | >64 | >64 |
| SA0110026 | >64 | >64 | >64 | 64 |
| SA0110036 | >64 | >64 | >64 | >64 |
| SA0110037 | >64 | >64 | >64 | >64 |
| SA0110040 | >64 | >64 | >64 | 16 |
| SA0110041 | >64 | >64 | >64 | >64 |
| SA0110043 | >64 | >64 | >64 | 32 |
| SA0110044 | >64 | >64 | >64 | >64 |
| SA0110067 | >64 | >64 | >64 | >64 |
| SA0110081 | >64 | >64 | >64 | >64 |
| SA0110082 | >64 | >64 | >64 | 64 |
| SA0110095 | >64 | >64 | >64 | 64 |
| SA0110098 | >64 | >64 | >64 | 32 |
| SA0110101 | >64 | >64 | >64 | 64 |
| SA0110113 | >64 | >64 | >64 | 64 |
| SA0110115 | >64 | >64 | >64 | 64 |
| SA0110117 | >64 | >64 | >64 | >64 |
| SA0110118 | >64 | >64 | >64 | >64 |
| SA0110120 | >64 | >64 | >64 | 32 |
| SA0110141 | >64 | >64 | >64 | 32 |
| SA0202089 | >64 | >64 | >64 | >64 |
| SA0202061 | >64 | >64 | >64 | >64 |
| SA0306004 | >64 | >64 | >64 | >64 |
| SA0110161 | >64 | >64 | >64 | >64 |
| SA0110170 | >64 | >64 | >64 | 32 |
| SA0110180 | >64 | >64 | >64 | 16 |
| SA0110184 | >64 | >64 | >64 | 16 |
| SA0110185 | >64 | >64 | >64 | 16 |
| SA0110193 | >64 | >64 | >64 | >64 |
| SA0110195 | >64 | >64 | >64 | >64 |
| SA0110196 | >64 | >64 | >64 | 16 |
| SA0110205 | >64 | >64 | >64 | 32 |
| SA0110206 | >64 | >64 | >64 | 4 |
| SA0110209 | >64 | >64 | >64 | 8 |
| SA0110210 | >64 | >64 | >64 | 16 |
| SA0110214 | >64 | >64 | >64 | 16 |
| SA0110215 | >64 | >64 | >64 | 16 |
| SA0110218 | >64 | >64 | >64 | 32 |
| SA0110225 | >64 | >64 | >64 | 16 |
| SA0110222 | >64 | >64 | >64 | 16 |
| SA0110223 | >64 | >64 | >64 | 8 |
| SA0110224 | >64 | >64 | >64 | 2 |
| SA0110239 | >64 | >64 | >64 | >64 |
| SA0110241 | >64 | >64 | >64 | >64 |
| SA0110252 | >64 | >64 | >64 | >64 |
| SA0110261 | >64 | >64 | >64 | >64 |
| SA0110264 | >64 | >64 | >64 | 1 |
| SA0110266 | >64 | >64 | >64 | 64 |
| SA0110273 | >64 | >64 | >64 | 8 |
| SA0110268 | >64 | >64 | >64 | 64 |
| SA0110279 | >64 | >64 | >64 | 0.25 |
| SA0111044 | >64 | >64 | >64 | >64 |
| SA0202097 | >64 | >64 | >64 | >64 |
| SA0202094 | >64 | >64 | >64 | >64 |
| levofloxacin | 1 | 2 | >8 | 0.03 |
| (positive control) | | | | |

### REFERENCES

(1) Yates, J. D.; Schaible, P. J. Nature 1962, 194, 183-184.
(2) (a) Delgado, G., Jr.; Neuhauser, M. M.; Bearden, D. T.; Danziger, L. H. Pharmacotherapy 2000, 20, 1469-1485; (b) Pavan, B. Curr. Opin. Invest. Drugs 2000, 7, 173-180; (c) Bonfiglio, G.; Furneri, P. M. Expert Opin. Invest. Drugs 2001, 10, 185-198. (3)
(a) Politano, A. D.; Sawyer, R. G. Curr. Opin. Invest. Drugs 2010, H, 225-236; (b) Pankuch, G. A.; Lin, G.; Clark, C.; Appelbaum, P. C. Antimicrob. Agents Chemother. 2011, 55, 1787-1791. (4) Allington, D. R.; Rivey, M. P. Clin. Ther. 2001, 23, 24-44. (5)(a) Hansen, J. L.; Moore, P. B.; Steitz, T. A. J. Mol. Bio. 2003, 330, 1061-1075; (b) Harms, J. M.; Schlünzen, F.; Fucini, P.; Bartels, H.; Yonath, A. BMC Biology 2004, 2, 4; (c) Noeske, J.; Huang, J.; Olivier, N. B.; Giacobbe, R. A.; Zambrowski, M.; Cate, J. H. D. Antimicrob. Agents Chemother. 2014, 58, 5269-5279; (d) Osterman, I. A.; Khabibullina, N. F.; Komarova, E. S.; Kasatsky, P.; Kartsev, V. G.; Bogdanov, A. A.; Dontsova, O. A.; Konevega, A. L.; Sergiev, P. V.; Polikanov, Y. S. Nucleic Acids Res. 2017.(6)(a) For a synthesis that proceeds in 2.1% yield over 17 steps that provided 7.6 mg of virginiamycin M1, see: Schlessinger, R. H.; Li, Y.-J. J. Am. Chem. Soc. 1996, 118, 3301-3302; (b) Wu, J.; Panek, J. S. Angew. Chem. Int. Ed. 2010, 49, 6165-6168. (7)(a) Tavares, F.; Lawson, J. P.; Meyers, A. I. J. Am. Chem. Soc. 1996, 118, 3303-3304; (b) Ghosh, A. K.; Liu, W. J. Org. Chem. 1997, 62, 7908-7909. (8) (a) Entwistle, D. A.; Jordan, S. I.; Montgomery, J.; Pattenden, G. J. Chem. Soc., Perkin Trans. 1 1996, 1315-1317; (b) Entwistle, D. A.; Jordan, S. I.; Montgomery, J.; Pattenden, G. Synthesis 1998, 1998, 603-612; (c) Dvorak, C. A.; Schmitz, W. D.; Poon, D. J.; Pryde, D. C.; Lawson, J. P.; Amos, R. A.; Meyers, A. I. Angew. Chem. Int. Ed 2000, 39, 1664-1666. (9) Simsek, S.; Kalesse, M. Tetrahedron Lett. 2009, 50, 3485-3488. (10) Although it is required in high loading, it is important to note that catalyst 10 is readily prepared in a single step from α,α-R-diphenylprolinol and boric acid, and its precursor can be recovered after use (see Example described herein, specifically Example 2). (11) Wood, R. D.; Ganem, B. Tetrahedron Lett. 1983, 24, 4391-4392. (12) Breuilles, P.; Uguen, D. Tetrahedron Lett. 1998, 39, 3149-3152. (13) (a) Hicks, J. D.; Hyde, A. M.; Cuezva, A. M.; Buchwald, S. L. J. Am. Chem. Soc. 2009, 131, 16720-16734; (b) For applications of JackiePhos in Stille reactions, see: Li, L.; Wang, C.-Y.; Huang, R.; Biscoe, M. R. Nat. Chem. 2013, 5, 607-612; (c) Zhu, F.; Rourke, M. J.; Yang, T.; Rodriguez, J.; Walczak, M. A. J. Am. Chem. Soc. 2016, 138, 12049-12052. (14) Chen, K.-M.; Hardtmann, G. E.; Prasad, K.; Repič, O.; Shapiro, M. J. Tetrahedron Lett. 1987, 28, 155-158. (15)
Devos, A.; Remion, J.; Frisque-Hesbain, A.-M.; Colens, A.; Ghosez, L. J. Chem. Soc., Chem. Comm. 1979, 1180-1181. (16) Wright, P. M.; Seiple, I. B.; Myers, A. G. Angew. Chem. Int. Ed. 2014, 53, 8840-8869. (17) Wu, J.; Panek, J. S. J. Org. Chem. 2011, 76, 9900-9918. (18) Wilson, D. N. Nat. Rev. Microbiol. 2014, 12, 35-48. (19) Stogios, P. J.; Kuhn, M. L.; Evdokimova, E.; Courvalin, P.; Anderson, W. F.; Savchenko, A. Antimicrob. Agents. Chemother. 2014, 58, 7083-7092.

1. A compound, or salt thereof, having the formula: 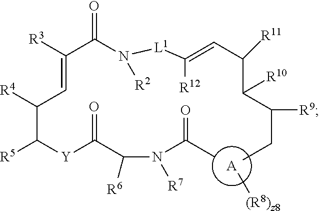
wherein
Y is —O— or —NH—;
L¹ is a bond, substituted or unsubstituted alkylene, or substituted or unsubstituted heteroalkylene;
R² is hydrogen or unsubstituted C₁-C₃ alkyl;
R³, R⁴, and R⁵ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —OPO₃H, —OSO₃H, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCH₂Cl, —OCH₂Br, —OCH₂I, —OCH₂F, —N₃, substituted or unsubstituted alkyl, substituted or unsubstituted heteroalkyl, or an amino acid side chain;
R⁷ is hydrogen, —CH₂COOH, —CONH₂, —OH, —SH, —NO₂, —NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ and R⁷ may optionally be joined to form an unsubstituted heterocycloalkyl or unsubstituted heteroaryl;
R⁸ is oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Ring A is cycloalkylene, heterocycloalkylene, arylene, or heteroarylene;
z8 is an integer from 0 to 10;
R⁹ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₂, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ and R¹² are independently hydrogen, substituted or unsubstituted C₁-C₃ alkyl, or substituted or unsubstituted 2 to 3 membered heteroalkyl; and
R¹¹ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₂, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
wherein the compound does not have the formula:

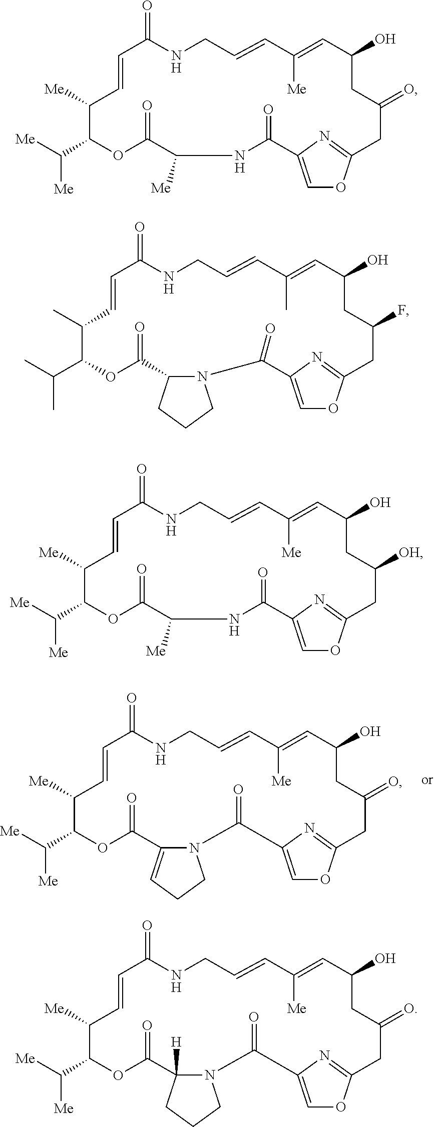

2. The compound of claim 1, wherein L¹ is a substituted or unsubstituted alkylene or substituted or unsubstituted heteroalkylene.

3. (canceled)

4. The compound of claim 1, wherein L¹ is a substituted or unsubstituted C₁-C₃ alkenylene.

5. The compound of claim 1, wherein R² is hydrogen.

6. The compound of claim 1, wherein R³ is hydrogen or substituted or unsubstituted alkyl.

7. (canceled)

8. (canceled)

9. The compound of claim 1, wherein R⁴ is hydrogen or a substituted or unsubstituted C₁-C₆ alkyl.

10. The compound of claim 1, wherein R⁵ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, or substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

11.-14. (canceled)

15. The compound of claim 1, wherein R⁵ is:

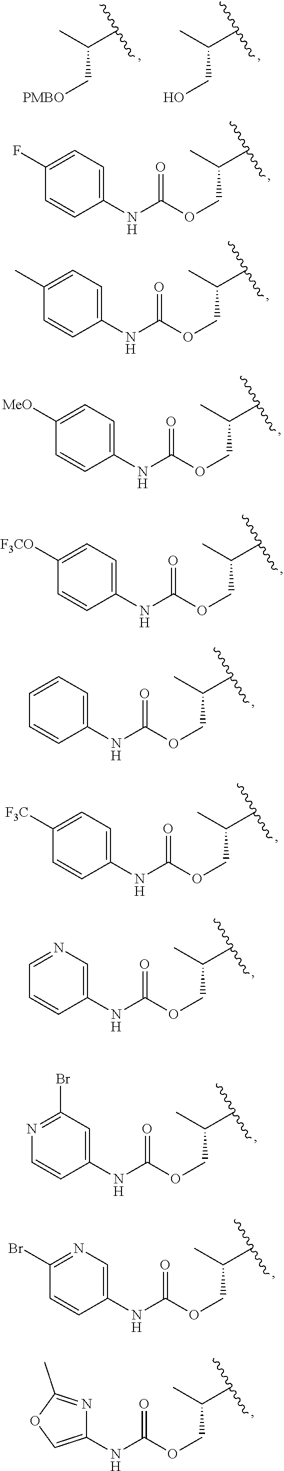

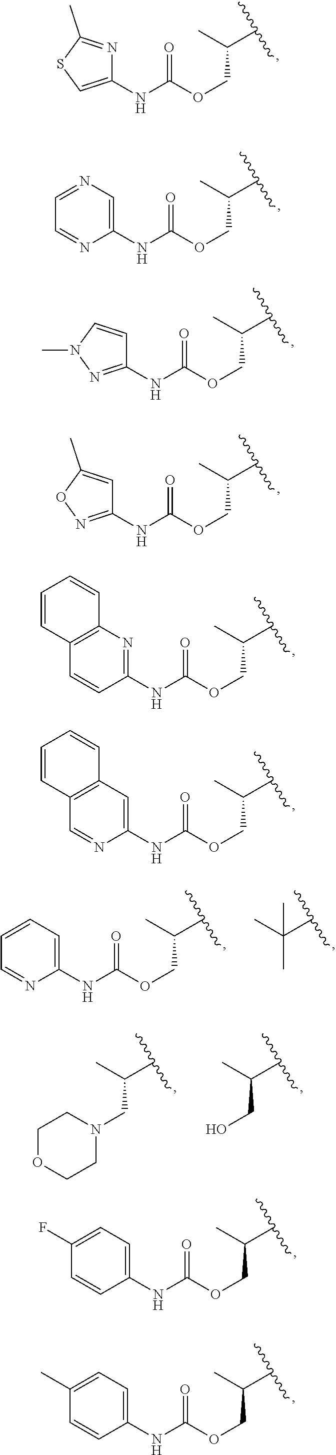

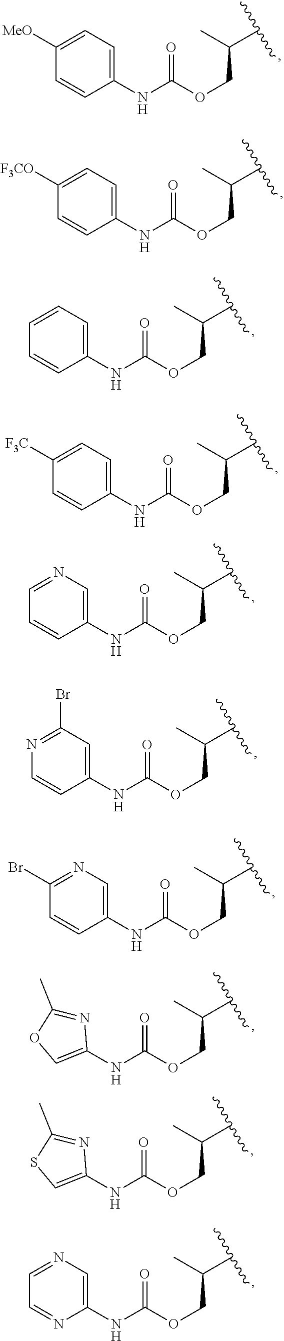

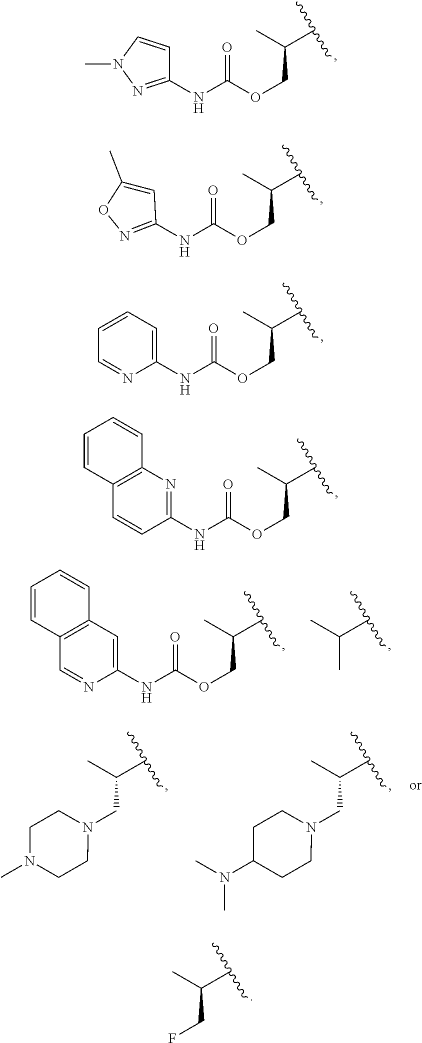

16. The compound of claim 1, wherein Y is —O—.

17. The compound of claim 1, wherein R⁶ is substituted or unsubstituted alkyl or substituted or unsubstituted heteroalkyl.

18. The compound of claim 1, wherein R⁶ is hydrogen,

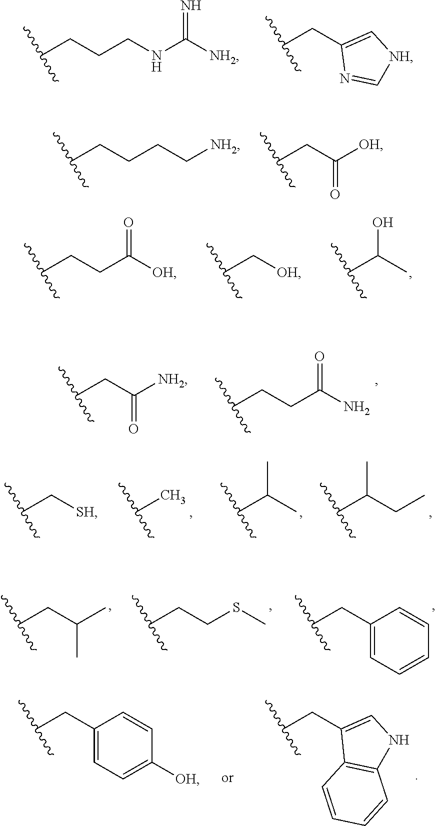

19. (canceled)

20. The compound of claim 1, wherein R⁶ and R⁷ are joined to form an unsubstituted heterocycloalkyl or unsubstituted heteroaryl.

21. (canceled)

22. (canceled)

23. The compound of claim 1, wherein R⁶ and R⁷ are joined to form an unsubstituted pyrrolidinyl or 2,3-dihydropyrrolyl.

24. The compound of claim 1, wherein R⁷ is hydrogen, substituted or unsubstituted alkyl, or substituted or unsubstituted heteroalkyl.

25. (canceled)

26. The compound of claim 1, wherein Ring A is C₃-C₆ cycloalkylene, 3 to 6 membered heterocycloalkylene, phenylene, or a 5 to 6 membered heteroarylene.

27.-29. (canceled)

30. The compound of claim 1, wherein z8 is 0.

31. The compound of claim 1, wherein R⁹ is halogen, oxo, —NH₂, unsubstituted alkyl, or unsubstituted heteroalkyl.

32. (canceled)

33. (canceled)

34. The compound of claim 1, wherein R¹¹ is —OH, —NH₂, or —SH.

35. (canceled)

36. (canceled)

37. The compound of claim 1, having the formula: 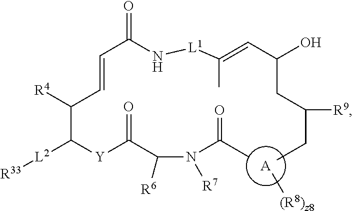
wherein
L² is substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R³³ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

38. The compound of claim 37, wherein -L²- is substituted or unsubstituted C₁-C₈ alkylene, substituted or unsubstituted 2 to 8 membered heteroalkylene, substituted or unsubstituted C₃-C₈ cycloalkylene, substituted or unsubstituted 3 to 8 membered heterocycloalkylene, substituted or unsubstituted C₆-C₁₀ arylene, or substituted or unsubstituted 5 to 10 membered heteroaryl ene.

39. (canceled)

40. (canceled)

41. The compound of claim 37, wherein -L²-R³³ is

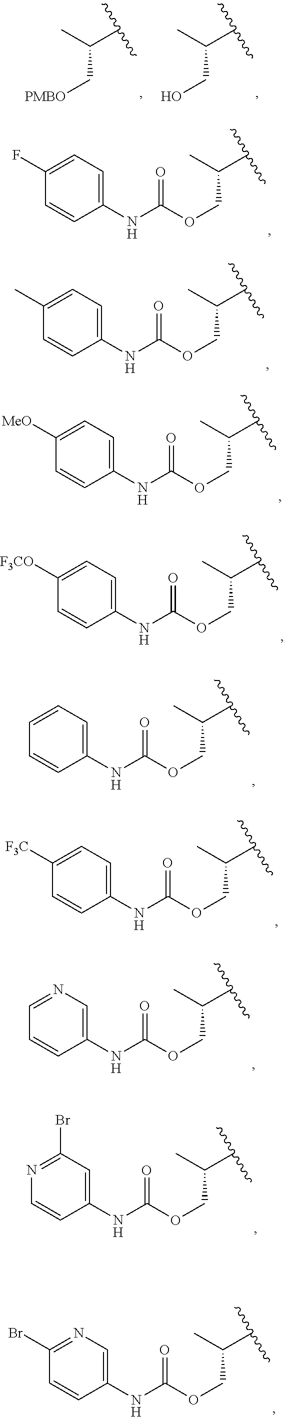

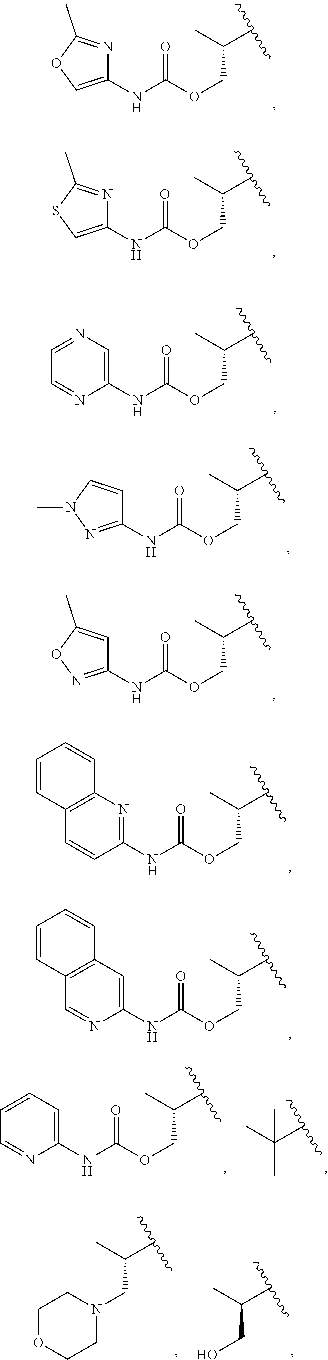

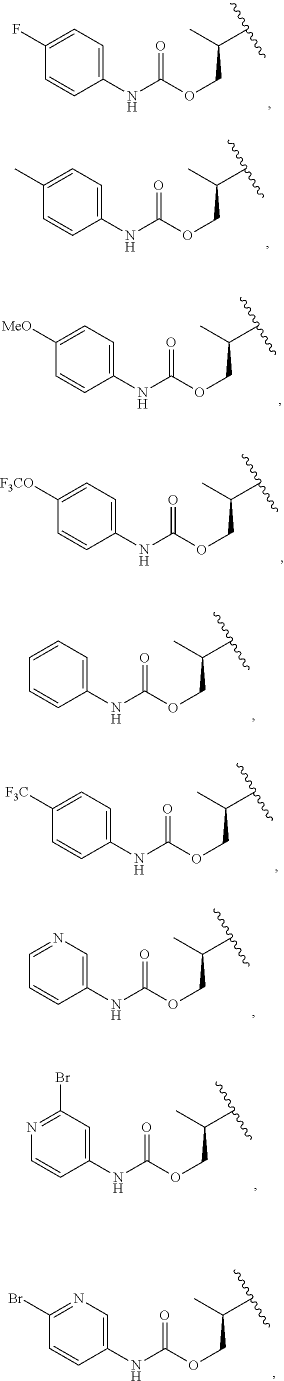

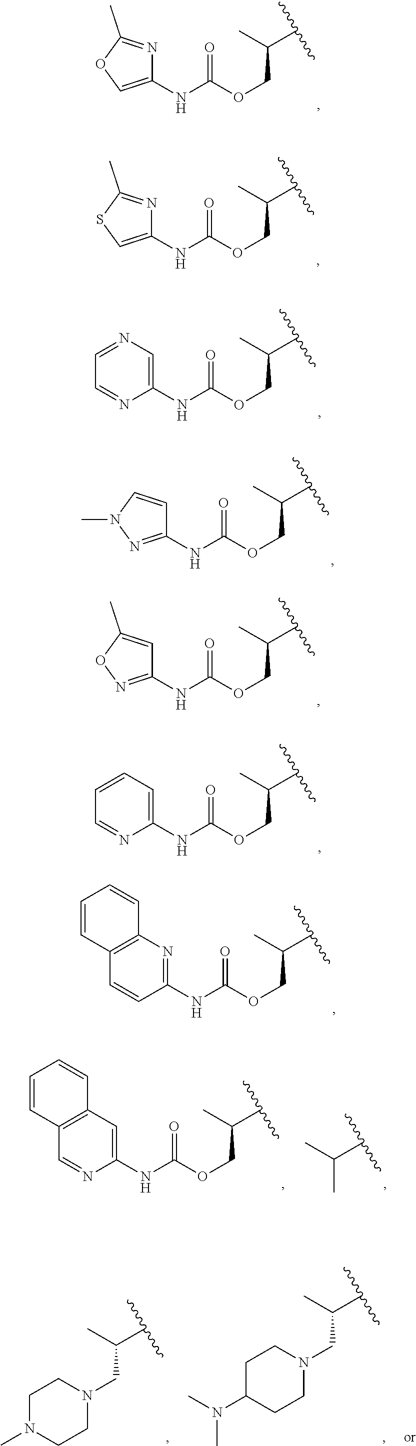

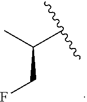 [SMILES: *[C@H](C)CF]

42. The compound of claim 1, wherein the compound has the formula:

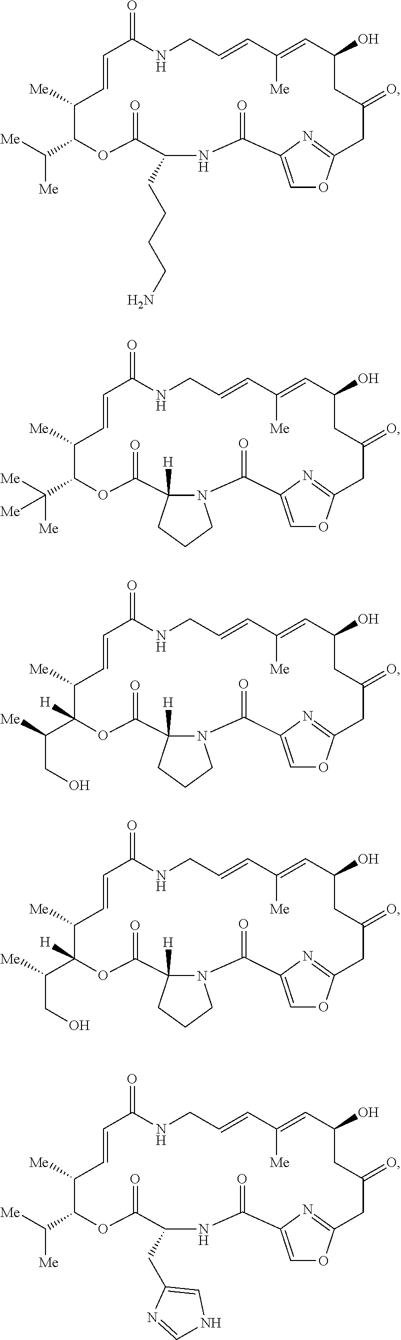 [SMILES: CC1=C\[C@@H](O)CC(=O)Cc2nc(co2)C(=O)N[C@H](CCCCN)C(=O)O[C@H](C(C)C)[C@H](C)/C=C/C(=O)NC\C=C\1 ; CC1=C\[C@@H](O)CC(=O)Cc2nc(co2)C(=O)N2CCC[C@@H]2C(=O)O[C@H](C(C)(C)C)[C@H](C)/C=C/C(=O)NC\C=C\1 ; CC1=C\[C@@H](O)CC(=O)Cc2nc(co2)C(=O)N2CCC[C@@H]2C(=O)O[C@H]([C@H](C)CO)[C@H](C)/C=C/C(=O)NC\C=C\1 ; CC1=C\[C@@H](O)CC(=O)Cc2nc(co2)C(=O)N2CCC[C@@H]2C(=O)O[C@H]([C@@H](C)CO)[C@H](C)/C=C/C(=O)NC\C=C\1 ; CC1=C\[C@@H](O)CC(=O)Cc2nc(co2)C(=O)N[C@H](Cc2c[nH]cn2)C(=O)O[C@H](C(C)C)[C@H](C)/C=C/C(=O)NC\C=C\1]

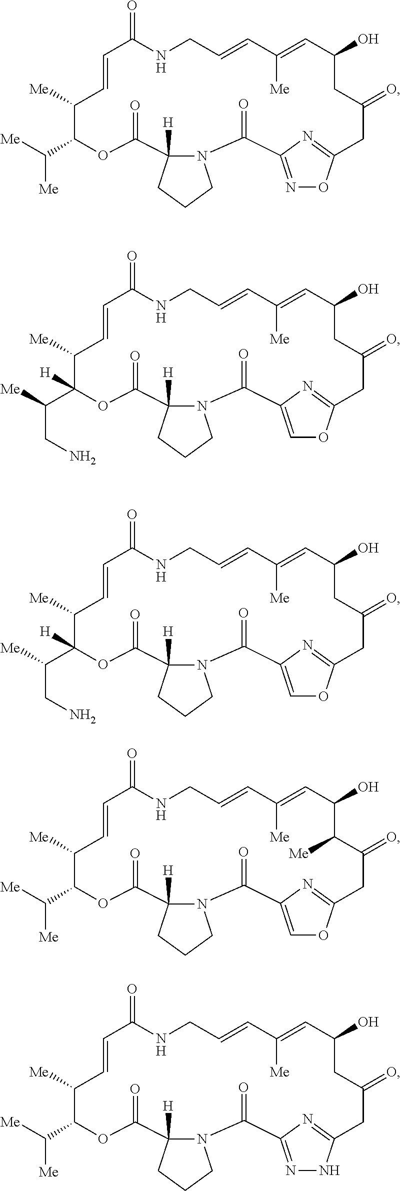 [SMILES: CC1=C\[C@@H](O)CC(=O)Cc2nc(no2)C(=O)N2CCC[C@@H]2C(=O)O[C@H](C(C)C)[C@H](C)/C=C/C(=O)NC\C=C\1 ; CC1=C\[C@@H](O)CC(=O)Cc2nc(co2)C(=O)N2CCC[C@@H]2C(=O)O[C@H]([C@H](C)CN)[C@H](C)/C=C/C(=O)NC\C=C\1 ; CC1=C\[C@@H](O)CC(=O)Cc2nc(co2)C(=O)N2CCC[C@@H]2C(=O)O[C@H]([C@@H](C)CN)[C@H](C)/C=C/C(=O)NC\C=C\1 ; CC1=C\[C@@H](O)[C@H](C)C(=O)Cc2nc(co2)C(=O)N2CCC[C@@H]2C(=O)O[C@H](C(C)C)[C@H](C)/C=C/C(=O)NC\C=C\1 ; CC1=C\[C@@H](O)CC(=O)Cc2nc(n[nH]2)C(=O)N2CCC[C@@H]2C(=O)O[C@H](C(C)C)[C@H](C)/C=C/C(=O)NC\C=C\1]

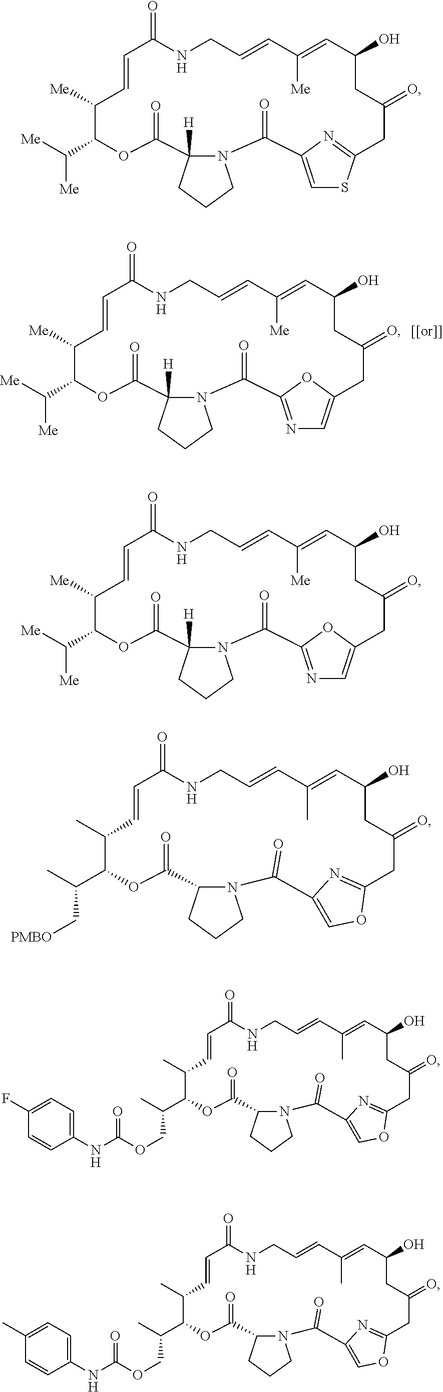

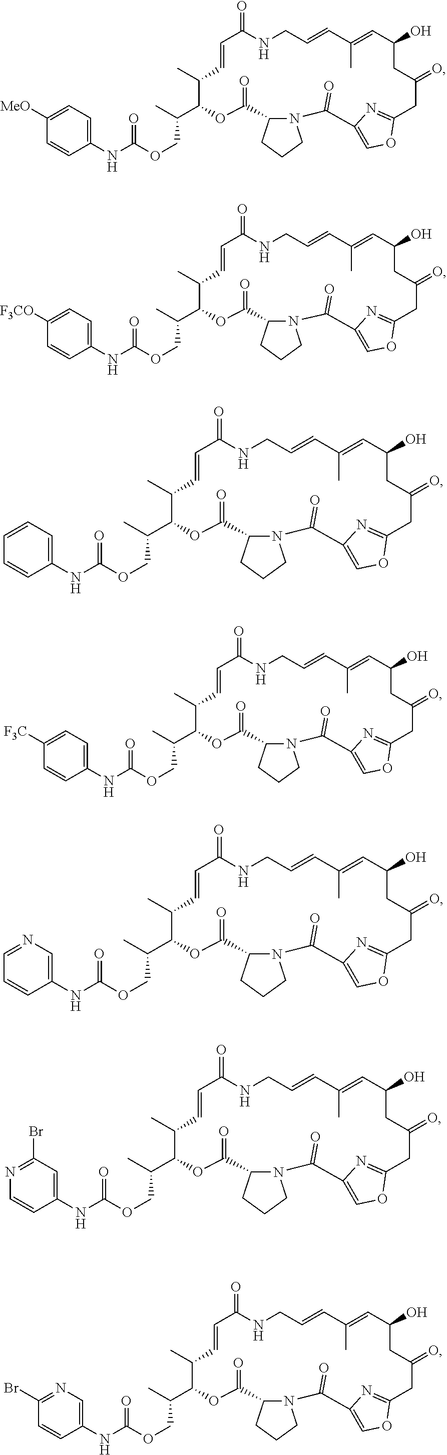

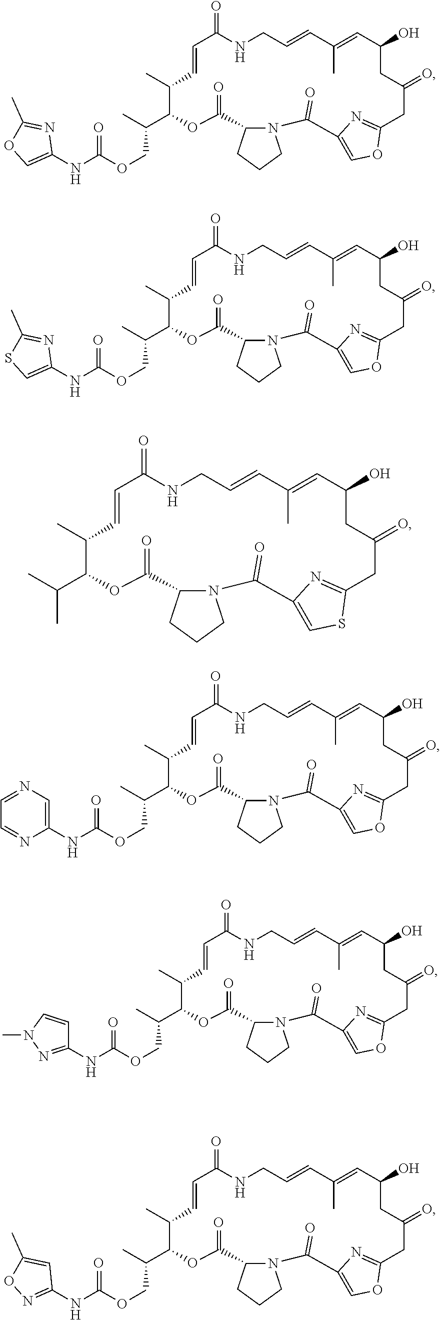

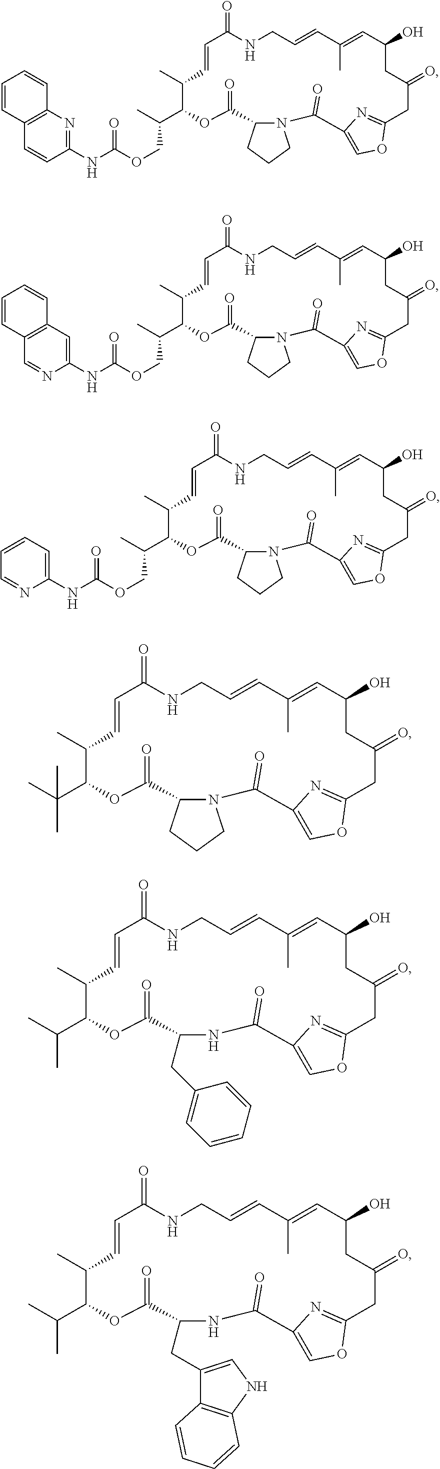

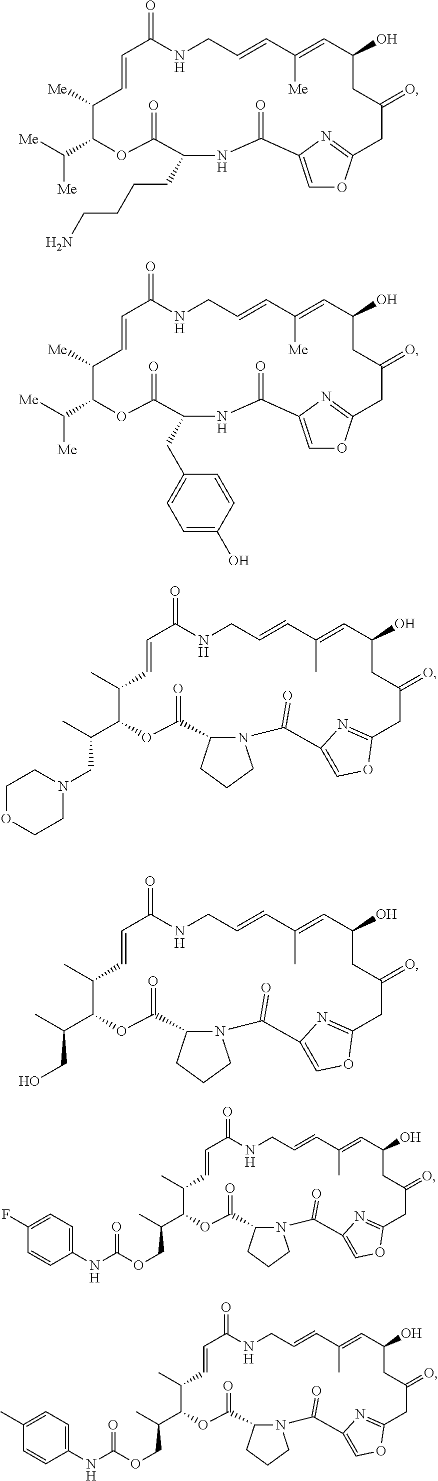

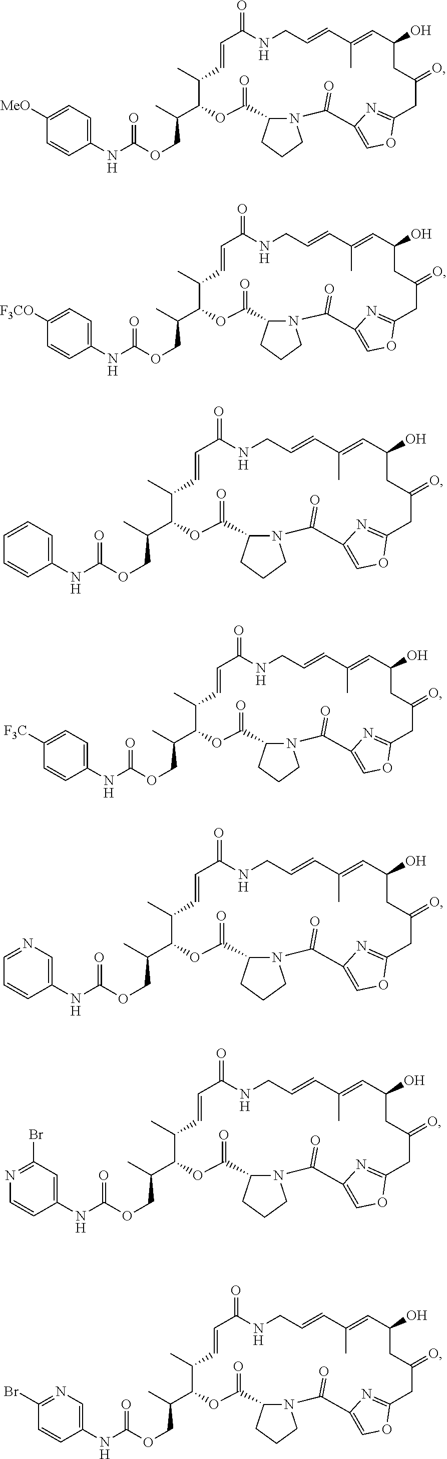

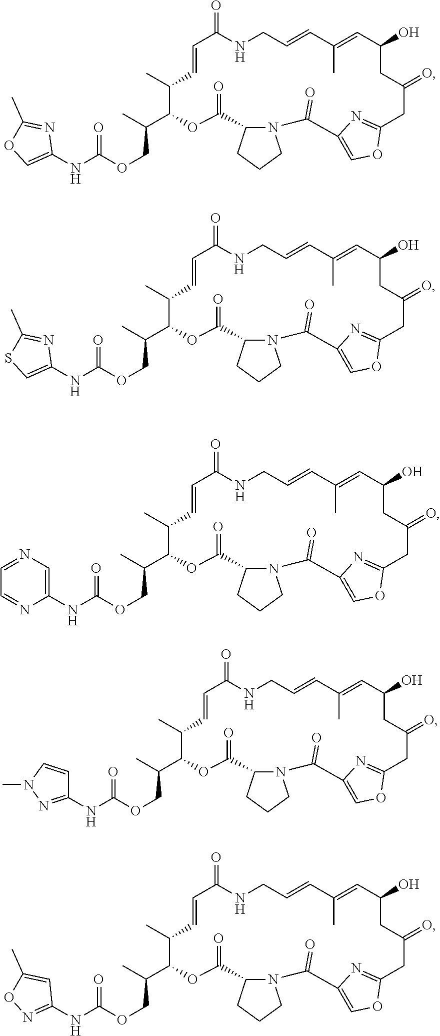

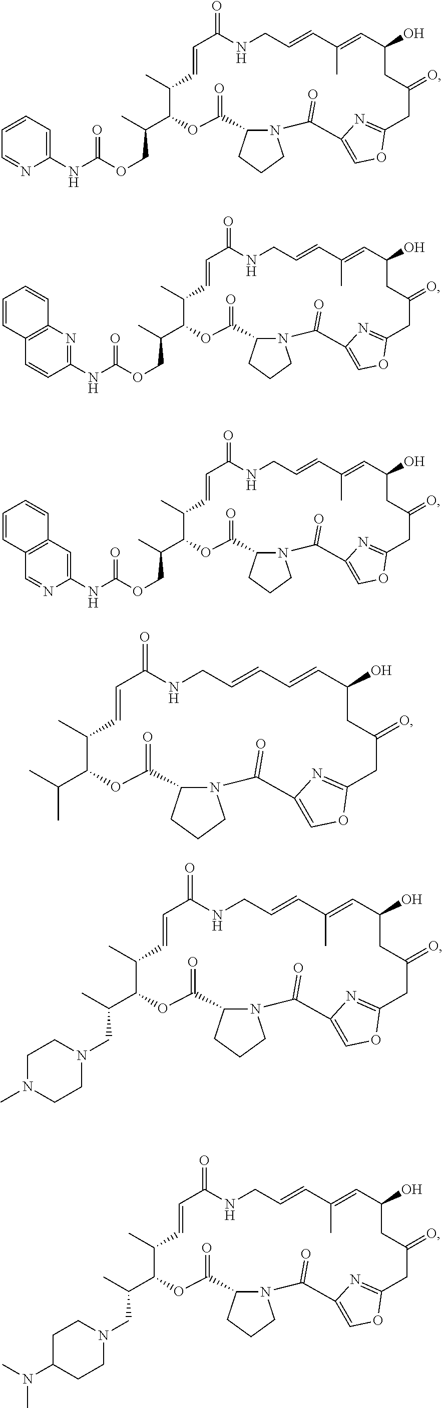

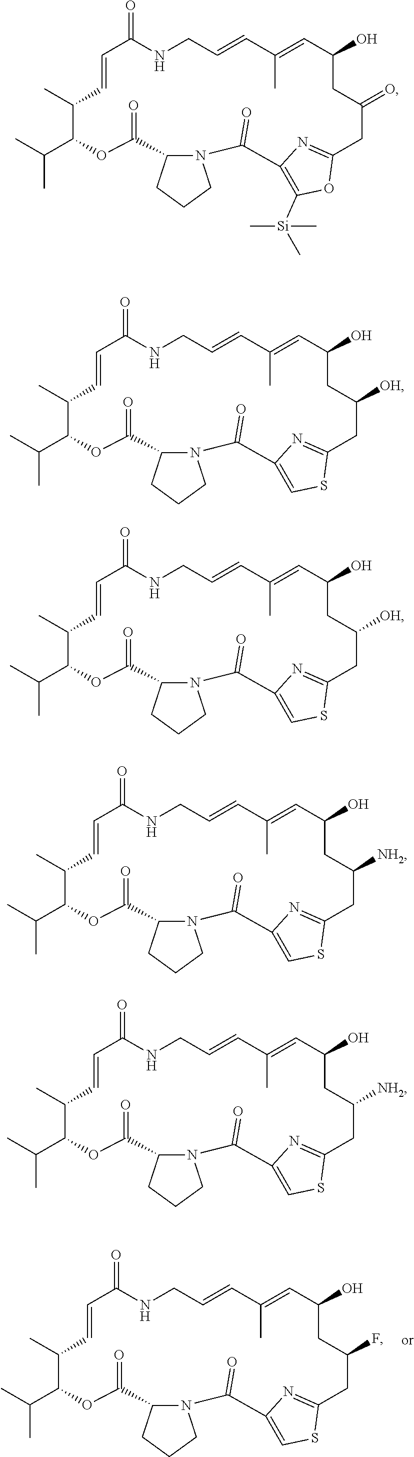

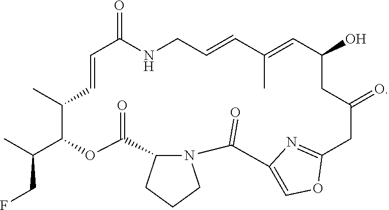 [SMILES: CC1=C\[C@@H](O)CC(=O)Cc2nc(co2)C(=O)N2CCC[C@@H]2C(=O)O[C@H]([C@H](C)CF)[C@H](C)/C=C/C(=O)NC\C=C\1]

43.-48. (canceled)

49. A method of making a compound, or salt thereof, having the formula: 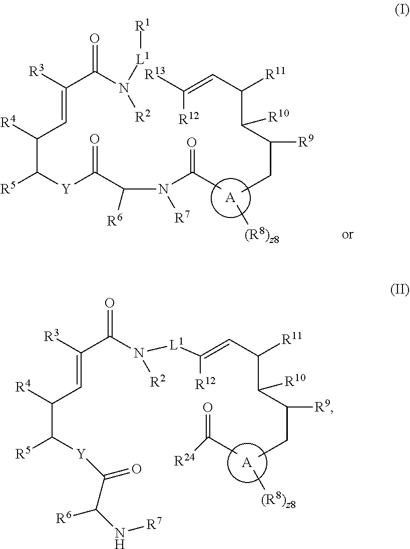
said method comprising mixing compound A and compound B together in a reaction vessel; wherein
compound A has the formula: 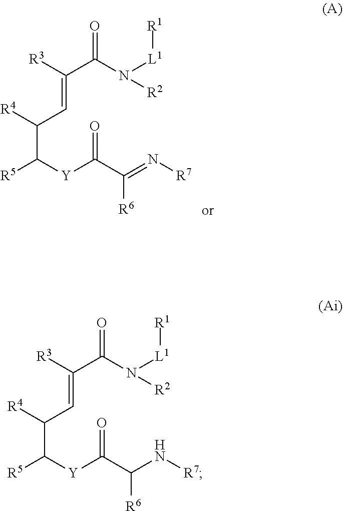
and
compound B has the formula: 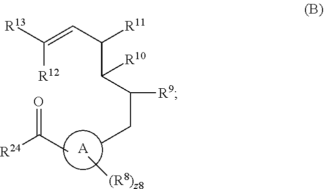
wherein
R¹, R¹³, and R²⁴ are independently bioconjugate reactive moieties or leaving groups;
Y is —O— or —NH—;
L¹ is a bond, substituted or unsubstituted alkylene, or substituted or unsubstituted heteroalkylene;
R² is hydrogen or unsubstituted C₁-C₃ alkyl;
R³, R⁴, and R⁵ are independently hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, —OPO₃H, —OSO₃H, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCH₂Cl, —OCH₂Br, —OCH₂I, —OCH₂F, —N₃, substituted or unsubstituted alkyl, substituted or unsubstituted heteroalkyl, or an amino acid side chain;
R⁷ is hydrogen, —CH₂COOH, —CONH₂, —OH, —SH, —NO₂, —NH₂, —NHNH₂, —ONH₂, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ and R⁷ may optionally be joined to form a substituted or unsubstituted heterocycloalkyl or substituted or unsubstituted heteroaryl;
R⁸ is oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Ring A is cycloalkylene, heterocycloalkylene, arylene, or heteroarylene;
z8 is an integer from 0 to 10;
R⁹ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₂, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ and R¹² are independently hydrogen, substituted or unsubstituted C₁-C₃ alkyl, or substituted or unsubstituted 2 to 3 membered heteroalkyl; and
R¹¹ is hydrogen, oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

50.-63. (canceled)

64. A pharmaceutical composition comprising a compound of claim 1, or pharmaceutically acceptable salt thereof, and a pharmaceutically acceptable excipient.

65. A method of treating an infectious disease, said method comprising administering to a subject in need thereof an effective amount of a compound of claim 1.

66.-70. (canceled)
